SEQUENCE LISTING (274 sequences)

SEQ ID NO: 1 - protein, 15 aa, synthetic construct
  REGION 1..15: note = CDR-L1
RASESVTILGSHLIH

SEQ ID NO: 2 - protein, 7 aa, synthetic construct
  REGION 1..7: note = CDR-L2
LASNVQT

SEQ ID NO: 3 - protein, 9 aa, synthetic construct
  REGION 1..9: note = CDR-L3
LQSRTIPRT

SEQ ID NO: 4 - protein, 5 aa, synthetic construct
  REGION 1..5: note = CDR-H1
DYSIN

SEQ ID NO: 5 - protein, 17 aa, synthetic construct
  REGION 1..17: note = CDR-H2
WINTETREPAYAYDFRG

SEQ ID NO: 6 - protein, 8 aa, synthetic construct
  REGION 1..8: note = CDR-H3
DYSYAMDY

SEQ ID NO: 7 - protein, 111 aa, synthetic construct
  REGION 1..111: note = Variable light (VL) Anti-BCMA
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIK

SEQ ID NO: 8 - protein, 117 aa, synthetic construct
  REGION 1..117: note = Variable heavy (VH) Anti-BCMA
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 9 - protein, 493 aa, synthetic construct
  REGION 1..493: note = BCMA CAR (with signal sequence)
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - DNA, 1485 bp, synthetic construct
  misc_feature 1..1485: note = BCMA CAR (with signal sequence)
  source 1..1485: mol_type = other DNA
atggcactccccgtcaccgcccttctcttgcccctcgccctgctgctgcatgctgccaggcccgacattgtgctcactcagtcacctcccagcctggccatgagcctgggaaaaagggccaccatctcctgtagagccagtgagtccgtcacaatcttggggagccatcttattcactggtatcagcagaagcccgggcagcctccaacccttcttattcagctcgcgtcaaacgtccagacgggtgtacctgccagattttctggtagcgggtcccgcactgattttacactgaccatagatccagtggaagaagacgatgtggccgtgtattattgtctgcagagcagaacgattcctcgcacatttggtgggggtactaagctggagattaagggaagcacgtccggctcagggaagccgggctccggcgagggaagcacgaaggggcaaattcagctggtccagagcggacctgagctgaaaaaacccggcgagactgttaagatcagttgtaaagcatctggctataccttcaccgactacagcataaattgggtgaaacgggcccctggaaagggcctcaaatggatgggttggatcaataccgaaactagggagcctgcttatgcatatgacttccgcgggagattcgccttttcactcgagacatctgcctctactgcttacctccaaataaacaacctcaagtatgaagatacagccacttacttttgcgccctcgactatagttacgccatggactactggggacagggaacctccgttaccgtcagttccgcggccgcaaccacaacacctgctccaaggccccccacacccgctccaactatagccagccaaccattgagcctcagacctgaagcttgcaggcccgcagcaggaggcgccgtccatacgcgaggcctggacttcgcgtgtgatatttatatttgggcccctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaagagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgctaatga

SEQ ID NO: 11 - protein, 184 aa, Homo sapiens
  REGION 1..184: note = Human BCMA
MLQMAGQCSQNEYFDSLLHACIPCQLRCSSNTPPLTCQRYCNASVTNSVKGTNAILWTCLGLSLIISLAVFVLMFLLRKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 12 - protein, 5 aa, synthetic construct
  REGION 1..5: note = Linker
DGGGS

SEQ ID NO: 13 - protein, 5 aa, synthetic construct
  REGION 1..5: note = Linker
TGEKP

SEQ ID NO: 14 - protein, 4 aa, synthetic construct
  REGION 1..4: note = Linker
GGRR

SEQ ID NO: 15 - protein, 5 aa, synthetic construct
  REGION 1..5: note = Linker
GGGGS

SEQ ID NO: 16 - protein, 14 aa, synthetic construct
  REGION 1..14: note = Linker
EGKSSGSGSESKVD

SEQ ID NO: 17 - protein, 18 aa, synthetic construct
  REGION 1..18: note = Linker
KESGSVSSEQLAQFRSLD

SEQ ID NO: 18 - protein, 8 aa, synthetic construct
  REGION 1..8: note = Linker
GGRRGGGS

SEQ ID NO: 19 - protein, 9 aa, synthetic construct
  REGION 1..9: note = Linker
LRQRDGERP

SEQ ID NO: 20 - protein, 12 aa, synthetic construct
  REGION 1..12: note = Linker
LRQKDGGGSERP

SEQ ID NO: 21 - protein, 16 aa, synthetic construct
  REGION 1..16: note = Linker
LRQKDGGGSGGGSERP

SEQ ID NO: 22 - protein, 18 aa, synthetic construct
  REGION 1..18: note = Linker
GSTSGSGKPGSGEGSTKG

SEQ ID NO: 23 - skipped

SEQ ID NO: 24 - protein, 7 aa, synthetic construct
  REGION 1..7: note = Protease cleavage site
ENLYFQG

SEQ ID NO: 25 - protein, 7 aa, synthetic construct
  REGION 1..7: note = Protease cleavage site
ENLYFQS

SEQ ID NO: 26 - protein, 19 aa, synthetic construct
  REGION 1..19: note = Self-cleaving polypeptide
LLNFDLLKLAGDVESNPGP

SEQ ID NO: 27 - protein, 19 aa, synthetic construct
  REGION 1..19: note = Self-cleaving polypeptide
TLNFDLLKLAGDVESNPGP

SEQ ID NO: 28 - protein, 14 aa, synthetic construct
  REGION 1..14: note = Self-cleaving polypeptide
LLKLAGDVESNPGP

SEQ ID NO: 29 - protein, 17 aa, synthetic construct
  REGION 1..17: note = Self-cleaving polypeptide
NFDLLKLAGDVESNPGP

SEQ ID NO: 30 - protein, 20 aa, synthetic construct
  REGION 1..20: note = Self-cleaving polypeptide
QLLNFDLLKLAGDVESNPGP

SEQ ID NO: 31 - protein, 24 aa, synthetic construct
  REGION 1..24: note = Self-cleaving polypeptide
APVKQTLNFDLLKLAGDVESNPGP

SEQ ID NO: 32 - protein, 40 aa, synthetic construct
  REGION 1..40: note = Self-cleaving polypeptide
VTELLYRMKRAETYCPRPLLAIHPTEARHKQKIVAPVKQT

SEQ ID NO: 33 - protein, 18 aa, synthetic construct
  REGION 1..18: note = Self-cleaving polypeptide
LNFDLLKLAGDVESNPGP

SEQ ID NO: 34 - protein, 40 aa, synthetic construct
  REGION 1..40: note = Self-cleaving polypeptide
LLAIHPTEARHKQKIVAPVKQTLNFDLLKLAGDVESNPGP

SEQ ID NO: 35 - protein, 33 aa, synthetic construct
  REGION 1..33: note = Self-cleaving polypeptide
EARHKQKIVAPVKQTLNFDLLKLAGDVESNPGP

SEQ ID NO: 36 - DNA, 7350 bp, synthetic construct
  misc_feature 1..7350: note = Vector encoding anti-BCMA CAR
  source 1..7350: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatcatatgccagcctatggtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgggtctctctggttagaccagatctgagcctgggagctctctggctaactagggaacccactgcttaagcctcaataaagcttgccttgagtgctcaaagtagtgtgtgcccgtctgttgtgtgactctggtaactagagatccctcagacccttttagtcagtgtggaaaatctctagcagtggcgcccgaacagggacttgaaagcgaaagtaaagccagaggagatctctcgacgcaggactcggcttgctgaagcgcgcacggcaagaggcgaggggcggcgactggtgagtacgccaaaaattttgactagcggaggctagaaggagagagtagggtgcgagagcgtcggtattaagcgggggagaattagataaatgggaaaaaattcggttaaggccagggggaaagaaacaatataaactaaaacatatagttagggcaagcagggagctagaacgattcgcagttaatcctggccttttagagacatcagaaggctgtagacaaatactgggacagctacaaccatcccttcagacaggatcagaagaacttagatcattatataatacaatagcagtcctctattgtgtgcatcaaaggatagatgtaaaagacaccaaggaagccttagataagatagaggaagagcaaaacaaaagtaagaaaaaggcacagcaagcagcagctgacacaggaaacaacagccaggtcagccaaaattaccctatagtgcagaacctccaggggcaaatggtacatcaggccatatcacctagaactttaaattaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggattaacacatggaaaagattagtaaaacaccatagctctagagcgatcccgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatccatctcgacggaatgaaagaccccacctgtaggtttggcaagctaggatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgattcacctgacgcgtctacgccaccatggcactccccgtcaccgcccttctcttgcccctcgccctgctgctgcatgctgccaggcccgacattgtgctcactcagtcacctcccagcctggccatgagcctgggaaaaagggccaccatctcctgtagagccagtgagtccgtcacaatcttggggagccatcttattcactggtatcagcagaagcccgggcagcctccaacccttcttattcagctcgcgtcaaacgtccagacgggtgtacctgccagattttctggtagcgggtcccgcactgattttacactgaccatagatccagtggaagaagacgatgtggccgtgtattattgtctgcagagcagaacgattcctcgcacatttggtgggggtactaagctggagattaagggaagcacgtccggctcagggaagccgggctccggcgagggaagcacgaaggggcaaattcagctggtccagagcggacctgagctgaaaaaacccggcgagactgttaagatcagttgtaaagcatctggctataccttcaccgactacagcataaattgggtgaaacgggcccctggaaagggcctcaaatggatgggttggatcaataccgaaactagggagcctgcttatgcatatgacttccgcgggagattcgccttttcactcgagacatctgcctctactgcttacctccaaataaacaacctcaagtatgaagatacagccacttacttttgcgccctcgactatagttacgccatggactactggggacagggaacctccgttaccgtcagttccgcggccgcaaccacaacacctgctccaaggccccccacacccgctccaactatagccagccaaccattgagcctcagacctgaagcttgcaggcccgcagcaggaggcgccgtccatacgcgaggcctggacttcgcgtgtgatatttatatttgggcccctttggccggaacatgtggggtgttgcttctctcccttgtgatcactctgtattgtaagcgcgggagaaagaagctcctgtacatcttcaagcagccttttatgcgacctgtgcaaaccactcaggaagaagatgggtgttcatgccgcttccccgaggaggaagaaggagggtgtgaactgagggtgaaattttctagaagcgccgatgctcccgcatatcagcagggtcagaatcagctctacaatgaattgaatctcggcaggcgagaagagtacgatgttctggacaagagacggggcagggatcccgagatggggggaaagccccggagaaaaaatcctcaggaggggttgtacaatgagctgcagaaggacaagatggctgaagcctatagcgagatcggaatgaaaggcgaaagacgcagaggcaaggggcatgacggtctgtaccagggtctctctacagccaccaaggacacttatgatgcgttgcatatgcaagccttgccaccccgctaatgacaggtacctttaagaccaatgacttacaaggcagctgtagatcttagccactttttaaaagaaaaggggggactggaagggctaattcactcccaaagaagacaagatctgctttttgcctgtactgggtctctctggttagaccagatctgagcctgggagctctctggctaactagggaacccactgcttaagcctcaataaagcttgccttgagtgcttcaatgtgtgtgttggttttttgtgtgtcgaaattctagcgattctagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggactagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgagcttgcatgccgacattgattattgactagtccctaagaaaccattcttatcatgacattaacctataaaaataggcgtatcacgaggccctttcgtc

SEQ ID NO: 37 - protein, 472 aa, synthetic construct
  REGION 1..472: note = anti-BCMA CAR (without signal sequence)
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 38 - protein, 246 aa, synthetic construct
  REGION 1..246: note = anti-BCMA scFv
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 39 - protein, 12 aa, Homo sapiens
  REGION 1..12: note = spacer (IgG4hinge)
ESKYGPPCPPCP

SEQ ID NO: 40 - DNA, 36 bp, Homo sapiens
  misc_feature 1..36: note = spacer (IgG4hinge)
  source 1..36: mol_type = unassigned DNA
gaatctaagtacggaccgccctgccccccttgccct

SEQ ID NO: 41 - protein, 119 aa, Homo sapiens
  REGION 1..119: note = Hinge-CH3 spacer
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 42 - protein, 229 aa, Homo sapiens
  REGION 1..229: note = Hinge-CH2-CH3 spacer
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 43 - protein, 282 aa, Homo sapiens
  REGION 1..282: note = IgD-hinge-Fc
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDH

SEQ ID NO: 44 - protein, 24 aa, synthetic construct
  REGION 1..24: note = T2A
LEGGGEGRGSLLTCGDVEENPGPR

SEQ ID NO: 45 - protein, 335 aa, synthetic construct
  REGION 1..335: note = tEGFR
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 46 - protein, 27 aa, Homo sapiens
  REGION 1..27: note = CD28 (amino acids 153-179 of Accession No. P10747)
FWVLVVVGGVLACYSLLVTVAFIIFWV

SEQ ID NO: 47 - protein, 66 aa, Homo sapiens
  REGION 1..66: note = CD28 (amino acids 114-179 of Accession No. P10747)
IEVMYPPPYLDNEKSNGTIIHVKGKHLCPSPLFPGPSKPFWVLVVVGGVLACYSLLVTVAFIIFWV

SEQ ID NO: 48 - protein, 41 aa, Homo sapiens
  REGION 1..41: note = CD28 (amino acids 180-220 of P10747)
RSKRSRLLHSDYMNMTPRRPGPTRKHYQPYAPPRDFAAYRS

SEQ ID NO: 49 - protein, 41 aa, Homo sapiens
  REGION 1..41: note = CD28 (LL to GG)
RSKRSRGGHSDYMNMTPRRPGPTRKHYQPYAPPRDFAAYRS

SEQ ID NO: 50 - protein, 42 aa, Homo sapiens
  REGION 1..42: note = 4-1BB (amino acids 214-255 of Q07011.1)
KRGRKKLLYIFKQPFMRPVQTTQEEDGCSCRFPEEEEGGCEL

SEQ ID NO: 51 - protein, 112 aa, Homo sapiens
  REGION 1..112: note = CD3 zeta
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 52 - protein, 112 aa, Homo sapiens
  REGION 1..112: note = CD3 zeta
RVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 53 - protein, 112 aa, Homo sapiens
  REGION 1..112: note = CD3 zeta
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 54 - protein, 10 aa, synthetic construct
  REGION 1..10: note = Linker
  REPEAT 5..9: note = SGGGG is repeated 5 times
PGGGSGGGGP

SEQ ID NO: 55 - protein, 17 aa, synthetic construct
  REGION 1..17: note = Linker
GSADDAKKDAAKKDGKS

SEQ ID NO: 56 - protein, 122 aa, synthetic construct
  REGION 1..122: note = Variable heavy (VH) Anti-BCMA
QIQLVQSGPDLKKPGETVKLSCKASGYTFTNFGMNWVKQAPGKGFKWMAWINTYTGESYFADDFKGRFAFSVETSATTAYLQINNLKTEDTATYFCARGEIYYGYDGGFAYWGQGTLVTVSA

SEQ ID NO: 57 - protein, 107 aa, synthetic construct
  REGION 1..107: note = Variable light (VL) Anti-BCMA
DVVMTQSHRFMSTSVGDRVSITCRASQDVNTAVSWYQQKPGQSPKLLIFSASYRYTGVPDRFTGSGSGADFTLTISSVQAEDLAVYYCQQHYSTPWTFGGGTKLDIK

SEQ ID NO: 58 - protein, 116 aa, synthetic construct
  REGION 1..116: note = Variable heavy (VH) Anti-BCMA
EVQLVQSGAEVKKPGESLKISCKGSGYSFTSYWIGWVRQMPGKGLEWMGIIYPGDSDTRYSPSFQGHVTISADKSISTAYLQWSSLKASDTAMYYCARYSGSFDNWGQGTLVTVSS

SEQ ID NO: 59 - protein, 111 aa, synthetic construct
  REGION 1..111: note = Variable light (VL) Anti-BCMA
SYELTQPPSASGTPGQRVTMSCSGTSSNIGSHSVNWYQQLPGTAPKLLIYTNNQRPSGVPDRFSGSKSGTSASLAISGLQSEDEADYYCAAWDGSLNGLVFGGGTKLTVLG

SEQ ID NO: 60 - protein, 118 aa, synthetic construct
  REGION 1..118: note = Variable heavy (VH) Anti-BCMA
EVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSS

SEQ ID NO: 61 - protein, 105 aa, synthetic construct
  REGION 1..105: note = Variable light (VL) Anti-BCMA
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLG

SEQ ID NO: 62 - protein, 4 aa, synthetic construct
  REGION 1..4: note = Linker
GGGS

SEQ ID NO: 63 - protein, 15 aa, synthetic construct
  REGION 1..15: note = Linker
GGGGSGGGGSGGGGS

SEQ ID NO: 64 - protein, 21 aa, synthetic construct
  REGION 1..21: note = Linker
SRGGGGSGGGGSGGGGSLEMA

SEQ ID NO: 65 - protein, 228 aa, Homo sapiens
  REGION 1..228: note = Hinge-CH2-CH3 spacer
ESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 66 - protein, 122 aa, synthetic construct
  REGION 1..122: note = Variable heavy (VH) Anti-BCMA
EVQLVQSGAEVKKPGSSVKVSCKASGGTFSSYAISWVRQAPGQGLEWMGRIIPILGIANYAQKFQGRVTMTEDTSTDTAYMELSSLRSEDTAVYYCARSGYSKSIVSYMDYWGQGTLVTVSS

SEQ ID NO: 67 - protein, 111 aa, synthetic construct
  REGION 1..111: note = Variable light (VL) Anti-BCMA
LPVLTQPPSTSGTPGQRVTVSCSGSSSNIGSNVVFWYQQLPGTAPKLVIYRNNQRPSGVPDRFSVSKSGTSASLAISGLRSEDEADYYCAAWDDSLSGYVFGTGTKVTVLG

SEQ ID NO: 68 - protein, 120 aa, synthetic construct
  REGION 1..120: note = Variable heavy (VH) Anti-BCMA
QVQLVQSGAEVKKPGSSVKVSCKASGGTFSSYAISWVRQAPGQGLEWMGRIIPILGTANYAQKFQGRVTITADESTSTAYMELSSLRSEDTAVYYCARSGYGSYRWEDSWGQGTLVTVSS

SEQ ID NO: 69 - protein, 112 aa, synthetic construct
  REGION 1..112: note = Variable light (VL) Anti-BCMA
QAVLTQPPSASGTPGQRVTISCSGSSSNIGSNYVFWYQQLPGTAPKLLIYSNNQRPSGVPDRFSGSKSGTSASLAISGLRSEDEADYYCAAWDDSLSASYVFGTGTKVTVLG

SEQ ID NO: 70 - protein, 118 aa, synthetic construct
  REGION 1..118: note = Variable heavy (VH) Anti-BCMA
QVQLVQSGAEVKKPGASVKVSCKASGYTFTDYYMHWVRQAPGQRLEWMGWINPNSGGTNYAQKFQDRITVTRDTSSNTGYMELTRLRSDDTAVYYCARSPYSGVLDKWGQGTLVTVSS

SEQ ID NO: 71 - protein, 112 aa, synthetic construct
  REGION 1..112: note = Variable light (VL) Anti-BCMA
QSVLTQPPSVSGAPGQRVTISCTGSSSNIGAGFDVHWYQQLPGTAPKLLIYGNSNRPSGVPDRFSGSKSGTSASLAITGLQAEDEADYYCQSYDSSLSGYVFGTGTKVTVLG

SEQ ID NO: 72 - protein, 17 aa, synthetic construct
  REGION 1..17: note = Linker
ASGGGGSGGRASGGGGS

SEQ ID NO: 73 - protein, 21 aa, synthetic construct
  REGION 1..21: note = CD8a signal peptide
MALPVTALLLPLALLLHAARP

SEQ ID NO: 74 - protein, 20 aa, synthetic construct
  REGION 1..20: note = signal peptide
METDTLLLWVLLLWVPGSTG

SEQ ID NO: 75 - protein, 118 aa, synthetic construct
  REGION 1..118: note = Variable heavy (VH) Anti-BCMA
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARAEMGAVFDIWGQGTMVTVSS

SEQ ID NO: 76 - protein, 107 aa, synthetic construct
  REGION 1..107: note = Variable light (VL) Anti-BCMA
EIVLTQSPATLSLSPGERATLSCRASQSVSRYLAWYQQKPGQAPRLLIYDASNRATGIPARFSGSGSGTDFTLTISSLEPEDFAVYYCQQRISWPFTFGGGTKVEIK

SEQ ID NO: 77 - protein, 122 aa, synthetic construct
  REGION 1..122: note = Variable heavy (VH) Anti-BCMA
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARDGTYLGGLWYFDLWGRGTLVTVSS

SEQ ID NO: 78 - protein, 112 aa, synthetic construct
  REGION 1..112: note = Variable light (VL) Anti-BCMA
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGLGLPLTFGGGTKVEIK

SEQ ID NO: 79 - protein, 116 aa, synthetic construct
  REGION 1..116: note = Variable heavy (VH) Anti-BCMA
QVQLVQSGAEVKKPGASVKVSCKASGYTFTSYYMHWVRQAPGQGLEWMGIINPGGGSTSYAQKFQGRVTMTRDTSTSTVYMELSSLRSEDTAVYYCARESWPMDVWGQGTTVTVSS

SEQ ID NO: 80 - protein, 106 aa, synthetic construct
  REGION 1..106: note = Variable light (VL) Anti-BCMA
EIVMTQSPATLSVSPGERATLSCRASQSVSSNLAWYQQKPGQAPRLLIYGASTRATGIPARFSGSGSGTEFTLTISSLQSEDFAVYYCQQYAAYPTFGGGTKVEIK

SEQ ID NO: 81 - protein, 122 aa, synthetic construct
  REGION 1..122: note = Variable heavy (VH) Anti-BCMA
QLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARGRGYATSLAFDIWGQGTMVTVSS

SEQ ID NO: 82 - protein, 107 aa, synthetic construct
  REGION 1..107: note = Variable light (VL) Anti-BCMA
EIVLTQSPATLSLSPGERATLSCRASQSVSSYLAWYQQKPGQAPRLLIYDASNRATGIPARFSGSGSGTDFTLTISSLEPEDFAVYYCQQRHVWPPTFGGGTKVEIK

SEQ ID NO: 83 - protein, 119 aa, synthetic construct
  REGION 1..119: note = Variable heavy (VH) Anti-BCMA
EVQLVESGGGLVQPGGSLRLSCAASGFTFSSYSMNWVRQAPGKGLEWVSTISSSSSTIYYADSVKGRFTISRDNAKNSLYLQMNSLRAEDTAVYYCARGSQEHLIFDYWGQGTLVTVSS

SEQ ID NO: 84 - protein, 107 aa, synthetic construct
  REGION 1..107: note = Variable light (VL) Anti-BCMA
EIVLTQSPATLSLSPGERATLSCRASQSVSRYLAWYQQKPGQAPRLLIYDASNRATGIPARFSGSGSGTDFTLTISSLEPEDFAVYYCQQRFYYPWTFGGGTKVEIK

SEQ ID NO: 85 - protein, 122 aa, synthetic construct
  REGION 1..122: note = Variable heavy (VH) Anti-BCMA
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARTDFWSGSPPGLDYWGQGTLVTVSS

SEQ ID NO: 86 - protein, 107 aa, synthetic construct
  REGION 1..107: note = Variable light (VL) Anti-BCMA
DIQLTQSPSSVSASVGDRVTITCRASQGISSWLAWYQQKPGKAPKLLIYGASSLQSGVPSRFSGSGSGTDFTLTISSLQPEDFATYYCQQIYTFPFTFGGGTKVEIK

SEQ ID NO: 87 - protein, 126 aa, synthetic construct
  REGION 1..126: note = Variable heavy (VH) Anti-BCMA
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARTPEYSSSIWHYYYGMDVWGQGTTVTVSS

SEQ ID NO: 88 - protein, 113 aa, synthetic construct
  REGION 1..113: note = Variable light (VL) Anti-BCMA
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQFAHTPFTFGGGTKVEIK

SEQ ID NO: 89 - protein, 123 aa, synthetic construct
  REGION 1..123: note = Variable heavy (VH) Anti-BCMA
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCVKGPLQEPPYDYGMDVWGQGTTVTVSS

SEQ ID NO: 90 - protein, 107 aa, synthetic construct
  REGION 1..107: note = Variable light (VL) Anti-BCMA
EIVMTQSPATLSVSPGERATLSCRASQSVSSNLAWYQQKPGQAPRLLIYSASTRATGIPARFSGSGSGTEFTLTISSLQSEDFAVYYCQQHHVWPLTFGGGTKVEIK

SEQ ID NO: 91 - protein, 121 aa, synthetic construct
  REGION 1..121: note = Variable heavy (VH) Anti-BCMA
QVQLVQSGAEVKKPGSSVKVSCKASGGTFSSYAISWVRQAPGQGLEWMGRIIPILGIANYAQKFQGRVTITADKSTSTAYMELSSLRSEDTAVYYCARGGYYSHDMWSEDWGQGTLVTVSS

SEQ ID NO: 92 - protein, 112 aa, synthetic construct
  REGION 1..112: note = Variable light (VL) Anti-BCMA
LPVLTQPPSASGTPGQRVTISCSGRSSNIGSNSVNWYRQLPGAAPKLLIYSNNQRPPGVPVRFSGSKSGTSASLAISGLQSEDEATYYCATWDDNLNVHYVFGTGTKVTVLG

SEQ ID NO: 93 - protein, 117 aa, synthetic construct
  REGION 1..117: note = Variable heavy (VH) Anti-BCMA
QVQLVQSGSELKKPGASVKVSCKASGYTFTDYSINWVRQAPGQGLEWMGWINTETREPAYAYDFRGRFVFSLDTSVSTAYLQISSLKAEDTAVYYCARDYSYAMDYWGQGTLVTVSS

SEQ ID NO: 94 - protein, 111 aa, synthetic construct
  REGION 1..111: note = Variable light (VL) Anti-BCMA
DIVLTQSPASLAVSLGERATINCRASESVSVIGAHLIHWYQQKPGQPPKLLIYLASNLETGVPARFSGSGSGTDFTLTISSLQAEDAAIYYCLQSRIFPRTFGQGTKLEIK

SEQ ID NO: 95 - protein, 115 aa, synthetic construct
  REGION 1..115: note = Variable heavy (VH) Anti-BCMA
EVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSS

SEQ ID NO: 96 - protein, 107 aa, synthetic construct
  REGION 1..107: note = Variable light (VL) Anti-BCMA
DIQLTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKVEIK

SEQ ID NO: 97 - protein, 120 aa, synthetic construct
  REGION 1..120: note = Variable heavy (VH) Anti-BCMA
QVQLVESGGGLVQPGRSLRLSCAASGFTFSNYAMSWVRQAPGKGLGWVSGISRSGENTYYADSVKGRFTISRDNSKNTLYLQMNSLRDEDTAVYYCARSPAHYYGGMDVWGQGTTVTVSS

SEQ ID NO: 98 - protein, 109 aa, synthetic construct
  REGION 1..109: note = Variable light (VL) Anti-BCMA
DIVLTQSPGTLSLSPGERATLSCRASQSISSSFLAWYQQKPGQAPRLLIYGASRRATGIPDRFSGSGSGTDFTLTISRLEPEDSAVYYCQQYHSSPSWTFGQGTKLEIK

SEQ ID NO: 99 - protein, 115 aa, synthetic construct
  REGION 1..115: note = Variable heavy (VH) Anti-BCMA
QVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSS

SEQ ID NO: 100 - protein, 107 aa, synthetic construct
  REGION 1..107: note = Variable light (VL) Anti-BCMA
DIRLTQSPSPLSASVGDRVTITCQASEDINKFLNWYHQTPGKAPKLLIYDASTLQTGVPSRFSGSGSGTDFTLTINSLQPEDIGTYYCQQYESLPLTFGGGTKVEIK

SEQ ID NO: 101 - protein, 115 aa, synthetic construct; identical to SEQ ID NO: 95
  REGION 1..115: note = Variable heavy (VH) Anti-BCMA

SEQ ID NO: 102 - protein, 109 aa, synthetic construct
  REGION 1..109: note = Variable light (VL) Anti-BCMA
EIVLTQSPGTLSLSPGERATLSCRASQSIGSSSLAWYQQKPGQAPRLLMYGASSRASGIPDRFSGSGSGTDFTLTISRLEPEDFAVYYCQQYAGSPPFTFGQGTKVEIK

SEQ ID NO: 103 - protein, 117 aa, synthetic construct
  REGION 1..117: note = Variable heavy (VH) Anti-BCMA
QIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSS

SEQ ID NO: 104 - protein, 111 aa, synthetic construct
  REGION 1..111: note = Variable light (VL) Anti-BCMA
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIK

SEQ ID NO: 105 - protein, 117 aa, synthetic construct
  REGION 1..117: note = Variable heavy (VH) Anti-BCMA
QIQLVQSGPELKKPGETVKISCKASGYTFTHYSMNWVKQAPGKGLKWMGRINTETGEPLYADDFKGRFAFSLETSASTAYLVINNLKNEDTATFFCSNDYLYSCDYWGQGTTLTVSS

SEQ ID NO: 106 - protein, 111 aa, synthetic construct
  REGION 1..111: note = Variable light (VL) Anti-BCMA
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIK

SEQ ID NO: 107 - protein, 119 aa, synthetic construct
  REGION 1..119: note = Variable heavy (VH) Anti-BCMA
QVQLVQSGAEVKKPGASVKVSCKASGYSFPDYYINWVRQAPGQGLEWMGWIYFASGNSEYNQKFTGRVTMTRDTSINTAYMELSSLTSEDTAVYFCASLYDYDWYFDVWGQGTMVTVSS

SEQ ID NO: 108 - protein, 112 aa, synthetic construct
  REGION 1..112: note = Variable light (VL) Anti-BCMA
DIVMTQTPLSLSVTPGQPASISCKSSQSLVHSNGNTYLHWYLQKPGQSPQLLIYKVSNRFSGVPDRFSGSGSGTDFTLKISRVEAEDVGIYYCSQSSIYPWTFGQGTKLEIK

SEQ ID NO: 109 - protein, 119 aa, synthetic construct
  REGION 1..119: note = Variable heavy (VH) Anti-BCMA
QVQLVQSGAEVKKPGASVKVSCKASGYSFPDYYINWVRQAPGQGLEWMGWIYFASGNSEYNQKFTGRVTMTRDTSSSTAYMELSSLRSEDTAVYFCASLYDYDWYFDVWGQGTMVTVSS

SEQ ID NO: 110 - protein, 112 aa, synthetic construct
  REGION 1..112: note = Variable light (VL) Anti-BCMA
DIVMTQTPLSLSVTPGEPASISCKSSQSLVHSNGNTYLHWYLQKPGQSPQLLIYKVSNRFSGVPDRFSGSGSGADFTLKISRVEAEDVGVYYCAETSHVPWTFGQGTKLEIK

SEQ ID NO: 111 - protein, 122 aa, synthetic construct
  REGION 1..122: note = Anti-BCMA sdAb
QVQLVESGGGLVQPGGSLRLSCEASGFTLDYYAIGWFRQAPGKEREGVICISRSDGSTYYADSVKGRFTISRDNAKKTVYLQMISLKPEDTAAYYCAAGADCSGYLRDYEFRGQGTQVTVSS

SEQ ID NO: 112 - protein, 39 aa, synthetic construct
  REGION 1..39: note = CD28 spacer
IEVMYPPPYLDNEKSNGTIIHVKGKHLCPSPLFPGPSKP

SEQ ID NO: 113 - protein, 25 aa, synthetic construct
  REGION 1..25: note = CD8a TM
IYIWAPLAGTCGVLLLSLVITLYCN

SEQ ID NO: 114 - protein, 30 aa, synthetic construct
  REGION 1..30: note = CD28 spacer (truncated)
LDNEKSNGTIIHVKGKHLCPSPLFPGPSKP

SEQ ID NO: 115 - protein, 46 aa, synthetic construct
  REGION 1..46: note = CD8a hinge
PTTTPAPRPPTPAPTIASQPLSLRPEACRPAAGGAVHTRGLDFACD

SEQ ID NO: 116 - protein, 45 aa, synthetic construct
  REGION 1..45: note = CD8a hinge
TTTPAPRPPTPAPTIASQPLSLRPEACRPAAGGAVHTRGLDFACD

SEQ ID NO: 117 - protein, 55 aa, synthetic construct
  REGION 1..55: note = CD8a hinge
FVPVFLPAKPTTTPAPRPPTPAPTIASQPLSLRPEACRPAAGGAVHTRGLDFACD

SEQ ID NO: 118 - protein, 39 aa, synthetic construct
  REGION 1..39: note = CTLA4 hinge
DTGLYICKVELMYPPPYYLGIGNGTQIYVIDPEPCPDSD

SEQ ID NO: 119 - protein, 24 aa, synthetic construct
  REGION 1..24: note = CTLA4 TM
FLLWILAAVSSGLFFYSFLLTAVS

SEQ ID NO: 120 - protein, 38 aa, synthetic construct
  REGION 1..38: note = PD-1 hinge
QIKESLRAELRVTERRAEVPTAHPSPSPRPAGQFQTLV

SEQ ID NO: 121 - protein, 21 aa, synthetic construct
  REGION 1..21: note = PD-1 TM
VGVVGGLLGSLVLLVWVLAVI

SEQ ID NO: 122 - protein, 16 aa, synthetic construct
  REGION 1..16: note = Fc(gamma)RIIIa hinge
GLAVSTISSFFPPGYQ

SEQ ID NO: 123 - protein, 231 aa, synthetic construct
  REGION 1..231: note = IgG1 hinge
EPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 124 - protein, 457 aa, synthetic construct
  REGION 1..457: note = anti-BCMA CAR
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARAEMGAVFDIWGQGTMVTVSSGSTSGSGKPGSGEGSTKGEIVLTQSPATLSLSPGERATLSCRASQSVSRYLAWYQQKPGQAPRLLIYDASNRATGIPARFSGSGSGTDFTLTISSLEPEDFAVYYCQQRISWPFTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 125 - protein, 457 aa, synthetic construct
  REGION 1..457: note = anti-BCMA CAR
EIVLTQSPATLSLSPGERATLSCRASQSVSRYLAWYQQKPGQAPRLLIYDASNRATGIPARFSGSGSGTDFTLTISSLEPEDFAVYYCQQRISWPFTFGGGTKVEIKRGSTSGSGKPGSGEGSTKGEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARAEMGAVFDIWGQGTMVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 126 - protein, 466 aa, synthetic construct
  REGION 1..466: note = anti-BCMA CAR
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARDGTYLGGLWYFDLWGRGTLVTVSSGSTSGSGKPGSGEGSTKGDIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGLGLPLTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 127 - protein, 466 aa, synthetic construct
  REGION 1..466: note = anti-BCMA CAR
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGLGLPLTFGGGTKVEIKRGSTSGSGKPGSGEGSTKGQVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARDGTYLGGLWYFDLWGRGTLVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 128 - protein, 454 aa, synthetic construct
  REGION 1..454: note = anti-BCMA CAR
QVQLVQSGAEVKKPGASVKVSCKASGYTFTSYYMHWVRQAPGQGLEWMGIINPGGGSTSYAQKFQGRVTMTRDTSTSTVYMELSSLRSEDTAVYYCARESWPMDVWGQGTTVTVSSGSTSGSGKPGSGEGSTKGEIVMTQSPATLSVSPGERATLSCRASQSVSSNLAWYQQKPGQAPRLLIYGASTRATGIPARFSGSGSGTEFTLTISSLQSEDFAVYYCQQYAAYPTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 129 - protein, 454 aa, synthetic construct
  REGION 1..454: note = anti-BCMA CAR
EIVMTQSPATLSVSPGERATLSCRASQSVSSNLAWYQQKPGQAPRLLIYGASTRATGIPARFSGSGSGTEFTLTISSLQSEDFAVYYCQQYAAYPTFGGGTKVEIKRGSTSGSGKPGSGEGSTKGQVQLVQSGAEVKKPGASVKVSCKASGYTFTSYYMHWVRQAPGQGLEWMGIINPGGGSTSYAQKFQGRVTMTRDTSTSTVYMELSSLRSEDTAVYYCARESWPMDVWGQGTTVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 130 - protein, 461 aa, synthetic construct
  REGION 1..461: note = anti-BCMA CAR
QLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARGRGYATSLAFDIWGQGTMVTVSSGSTSGSGKPGSGEGSTKGEIVLTQSPATLSLSPGERATLSCRASQSVSSYLAWYQQKPGQAPRLLIYDASNRATGIPARFSGSGSGTDFTLTISSLEPEDFAVYYCQQRHVWPPTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 131 - protein, 461 aa, synthetic construct
  REGION 1..461: note = anti-BCMA CAR
EIVLTQSPATLSLSPGERATLSCRASQSVSSYLAWYQQKPGQAPRLLIYDASNRATGIPARFSGSGSGTDFTLTISSLEPEDFAVYYCQQRHVWPPTFGGGTKVEIKRGSTSGSGKPGSGEGSTKGQLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARGRGYATSLAFDIWGQGTMVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 132 - protein, 458 aa, synthetic construct
  REGION 1..458: note = anti-BCMA CAR
EVQLVESGGGLVQPGGSLRLSCAASGFTFSSYSMNWVRQAPGKGLEWVSTISSSSSTIYYADSVKGRFTISRDNAKNSLYLQMNSLRAEDTAVYYCARGSQEHLIFDYWGQGTLVTVSSGSTSGSGKPGSGEGSTKGEIVLTQSPATLSLSPGERATLSCRASQSVSRYLAWYQQKPGQAPRLLIYDASNRATGIPARFSGSGSGTDFTLTISSLEPEDFAVYYCQQRFYYPWTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 133 - protein, 458 aa, synthetic construct
  REGION 1..458: note = anti-BCMA CAR
EIVLTQSPATLSLSPGERATLSCRASQSVSRYLAWYQQKPGQAPRLLIYDASNRATGIPARFSGSGSGTDFTLTISSLEPEDFAVYYCQQRFYYPWTFGGGTKVEIKRGSTSGSGKPGSGEGSTKGEVQLVESGGGLVQPGGSLRLSCAASGFTFSSYSMNWVRQAPGKGLEWVSTISSSSSTIYYADSVKGRFTISRDNAKNSLYLQMNSLRAEDTAVYYCARGSQEHLIFDYWGQGTLVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 134 - protein, 461 aa, synthetic construct
  REGION 1..461: note = anti-BCMA CAR
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARTDFWSGSPPGLDYWGQGTLVTVSSGSTSGSGKPGSGEGSTKGDIQLTQSPSSVSASVGDRVTITCRASQGISSWLAWYQQKPGKAPKLLIYGASSLQSGVPSRFSGSGSGTDFTLTISSLQPEDFATYYCQQIYTFPFTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 135 - protein, 461 aa, synthetic construct
  REGION 1..461: note = anti-BCMA CAR
DIQLTQSPSSVSASVGDRVTITCRASQGISSWLAWYQQKPGKAPKLLIYGASSLQSGVPSRFSGSGSGTDFTLTISSLQPEDFATYYCQQIYTFPFTFGGGTKVEIKRGSTSGSGKPGSGEGSTKGQVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARTDFWSGSPPGLDYWGQGTLVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 136 - protein, 471 aa, synthetic construct
  REGION 1..471: note = anti-BCMA CAR
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARTPEYSSSIWHYYYGMDVWGQGTTVTVSSGSTSGSGKPGSGEGSTKGDIVMTQSPDSLAVSLGERATINCKSSQSVLYSSNNKNYLAWYQQKPGQPPKLLIYWASTRESGVPDRFSGSGSGTDFTLTISSLQAEDVAVYYCQQFAHTPFTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 137 - protein, 471 aa, synthetic construct
  REGION 1..471: note = anti-BCMA CAR
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQFAHTPFTFGGGTKVEIKRGSTSGSGKPGSGEGSTKGQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARTPEYSSSIWHYYYGMDVWGQGTTVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 138 - protein, 462 aa, synthetic construct
  REGION 1..462: note = anti-BCMA CAR
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCVKGPLQEPPYDYGMDVWGQGTTVTVSSGSTSGSGKPGSGEGSTKGEIVMTQSPATLSVSPGERATLSCRASQSVSSNLAWYQQKPGQAPRLLIYSASTRATGIPARFSGSGSGTEFTLTISSLQSEDFAVYYCQQHHVWPLTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 139 - protein, 462 aa, synthetic construct
  REGION 1..462: note = anti-BCMA CAR
EIVMTQSPATLSVSPGERATLSCRASQSVSSNLAWYQQKPGQAPRLLIYSASTRATGIPARFSGSGSGTEFTLTISSLQSEDFAVYYCQQHHVWPLTFGGGTKVEIKRGSTSGSGKPGSGEGSTKGQVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCVKGPLQEPPYDYGMDVWGQGTTVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 140 - protein, 466 aa, synthetic construct
  REGION 1..466: note = anti-BCMA CAR
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGSRGGGGSGGGGSGGGGSLEMAEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 141 - protein, 473 aa, synthetic construct
  REGION 1..473: note = anti-BCMA CAR
QSVLTQPPSVSGAPGQRVTISCTGSSSNIGAGFDVHWYQQLPGTAPKLLIYGNSNRPSGVPDRFSGSKSGTSASLAITGLQAEDEADYYCQSYDSSLSGYVFGTGTKVTVLGSRGGGGSGGGGSGGGGSLEMAQVQLVQSGAEVKKPGASVKVSCKASGYTFTDYYMHWVRQAPGQRLEWMGWINPNSGGTNYAQKFQDRITVTRDTSSNTGYMELTRLRSDDTAVYYCARSPYSGVLDKWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 142 - protein, 470 aa, synthetic construct
  REGION 1..470: note = anti-BCMA CAR
SYELTQPPSASGTPGQRVTMSCSGTSSNIGSHSVNWYQQLPGTAPKLLIYTNNQRPSGVPDRFSGSKSGTSASLAISGLQSEDEADYYCAAWDGSLNGLVFGGGTKLTVLGSRGGGGSGGGGSGGGGSLEMAEVQLVQSGAEVKKPGESLKISCKGSGYSFTSYWIGWVRQMPGKGLEWMGIIYPGDSDTRYSPSFQGHVTISADKSISTAYLQWSSLKASDTAMYYCARYSGSFDNWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 143 - protein, 476 aa, synthetic construct
  REGION 1..476: note = anti-BCMA CAR
LPVLTQPPSASGTPGQRVTISCSGRSSNIGSNSVNWYRQLPGAAPKLLIYSNNQRPPGVPVRFSGSKSGTSASLAISGLQSEDEATYYCATWDDNLNVHYVFGTGTKVTVLGSRGGGGSGGGGSGGGGSLEMAQVQLVQSGAEVKKPGSSVKVSCKASGGTFSSYAISWVRQAPGQGLEWMGRIIPILGIANYAQKFQGRVTITADKSTSTAYMELSSLRSEDTAVYYCARGGYYSHDMWSEDWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 144 - protein, 475 aa, synthetic construct
  REGION 1..475: note = anti-BCMA CAR
QAVLTQPPSASGTPGQRVTISCSGSSSNIGSNYVFWYQQLPGTAPKLLIYSNNQRPSGVPDRFSGSKSGTSASLAISGLRSEDEADYYCAAWDDSLSASYVFGTGTKVTVLGSRGGGGSGGGGSGGGGSLEMAQVQLVQSGAEVKKPGSSVKVSCKASGGTFSSYAISWVRQAPGQGLEWMGRIIPILGTANYAQKFQGRVTITADESTSTAYMELSSLRSEDTAVYYCARSGYGSYRWEDSWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 145 - protein, 482 aa, synthetic construct
  REGION 1..482: note = anti-BCMA CAR
LPVLTQPPSASGTPGQRVTISCSGRSSNIGSNSVNWYRQLPGAAPKLLIYSNNQRPPGVPVRFSGSKSGTSASLAISGLQSEDEATYYCATWDDNLNVHYVFGTGTKVTVLGSRGGGGSGGGGSGGGGSLEMAQVQLVQSGAEVKKPGSSVKVSCKASGGTFSSYAISWVRQAPGQGLEWMGRIIPILGIANYAQKFQGRVTITADKSTSTAYMELSSLRSEDTAVYYCARGGYYSHDMWSEDWGQGTLVTVSSAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 146 - protein, 476 aa, synthetic construct
  REGION 1..476: note = anti-BCMA CAR
SYELTQPPSASGTPGQRVTMSCSGTSSNIGSHSVNWYQQLPGTAPKLLIYTNNQRPSGVPDRFSGSKSGTSASLAISGLQSEDEADYYCAAWDGSLNGLVFGGGTKLTVLGSRGGGGSGGGGSGGGGSLEMAEVQLVQSGAEVKKPGESLKISCKGSGYSFTSYWIGWVRQMPGKGLEWMGIIYPGDSDTRYSPSFQGHVTISADKSISTAYLQWSSLKASDTAMYYCARYSGSFDNWGQGTLVTVSSAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 147 - protein, 481 aa, synthetic construct
  REGION 1..481: note = anti-BCMA CAR
QAVLTQPPSASGTPGQRVTISCSGSSSNIGSNYVFWYQQLPGTAPKLLIYSNNQRPSGVPDRFSGSKSGTSASLAISGLRSEDEADYYCAAWDDSLSASYVFGTGTKVTVLGSRGGGGSGGGGSGGGGSLEMAQVQLVQSGAEVKKPGSSVKVSCKASGGTFSSYAISWVRQAPGQGLEWMGRIIPILGTANYAQKFQGRVTITADESTSTAYMELSSLRSEDTAVYYCARSGYGSYRWEDSWGQGTLVTVSSAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 148 - protein, 479 aa, synthetic construct
  REGION 1..479: note = anti-BCMA CAR
QSVLTQPPSVSGAPGQRVTISCTGSSSNIGAGFDVHWYQQLPGTAPKLLIYGNSNRPSGVPDRFSGSKSGTSASLAITGLQAEDEADYYCQSYDSSLSGYVFGTGTKVTVLGSRGGGGSGGGGSGGGGSLEMAQVQLVQSGAEVKKPGASVKVSCKASGYTFTDYYMHWVRQAPGQRLEWMGWINPNSGGTNYAQKFQDRITVTRDTSSNTGYMELTRLRSDDTAVYYCARSPYSGVLDKWGQGTLVTVSSAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 149 - protein, 472 aa, synthetic construct
  REGION 1..472: note = anti-BCMA CAR
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGSRGGGGSGGGGSGGGGSLEMAEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSSAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 150 - protein, 472 aa, synthetic construct
  REGION 1..472: note = anti-BCMA CAR
DIVLTQSPASLAVSLGERATINCRASESVSVIGAHLIHWYQQKPGQPPKLLIYLASNLETGVPARFSGSGSGTDFTLTISSLQAEDAAIYYCLQSRIFPRTFGQGTKLEIKGSTSGSGKPGSGEGSTKGQVQLVQSGSELKKPGASVKVSCKASGYTFTDYSINWVRQAPGQGLEWMGWINTETREPAYAYDFRGRFVFSLDTSVSTAYLQISSLKAEDTAVYYCARDYSYAMDYWGQGTLVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 151 - protein, 466 aa, synthetic construct
  REGION 1..466: note = anti-BCMA CAR
DIVLTQSPASLAVSLGERATINCRASESVSVIGAHLIHWYQQKPGQPPKLLIYLASNLETGVPARFSGSGSGTDFTLTISSLQAEDAAIYYCLQSRIFPRTFGQGTKLEIKGSTSGSGKPGSGEGSTKGQVQLVQSGSELKKPGASVKVSCKASGYTFTDYSINWVRQAPGQGLEWMGWINTETREPAYAYDFRGRFVFSLDTSVSTAYLQISSLKAEDTAVYYCARDYSYAMDYWGQGTLVTVSSAAADTGLYICKVELMYPPPYYLGIGNGTQIYVIDPEPCPDSDFLLWILAAVSSGLFFYSFLLTAVSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 152 - protein, 464 aa, synthetic construct
  REGION 1..464: note = anti-BCMA CAR
DIVLTQSPASLAVSLGERATINCRASESVSVIGAHLIHWYQQKPGQPPKLLIYLASNLETGVPARFSGSGSGTDFTLTISSLQAEDAAIYYCLQSRIFPRTFGQGTKLEIKGSTSGSGKPGSGEGSTKGQVQLVQSGSELKKPGASVKVSCKASGYTFTDYSINWVRQAPGQGLEWMGWINTETREPAYAYDFRGRFVFSLDTSVSTAYLQISSLKAEDTAVYYCARDYSYAMDYWGQGTLVTVSSAAAQIKESLRAELRVTERRAEVPTAHPSPSPRPAGQFQTLVVGVVGGLLGSLVLLVWVLAVICSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 153 - protein, 653 aa, synthetic construct
  REGION 1..653: note = anti-BCMA CAR
EVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAKVDGDYTEDYWGQGTLVTVSSGGGGSGGGGSGGGGSQSALTQPASVSGSPGQSITISCTGSSSDVGKYNLVSWYQQPPGKAPKLIIYDVNKRPSGVSNRFSGSKSGNTATLTISGLQGDDEADYYCSSYGGSRSYVFGTGTKVTVLESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 154 - protein, 650 aa, synthetic construct
  REGION 1..650: note = anti-BCMA CAR
EVQLVQSGGGLVQPGRSLRLSCTASGFTFGDYAMSWFKQAPGKGLEWVGFIRSKAYGGTTEYAASVKGRFTISRDDSKSIAYLQMNSLKTEDTAVYYCAAWSAPTDYWGQGTLVTVSSGGGGSGGGGSGGGGSDIQMTQSPAFLSASVGDRVTVTCRASQGISNYLAWYQQKPGNAPRLLIYSASTLQSGVPSRFRGTGYGTEFSLTIDSLQPEDFATYYCQQSYTSRQTFGPGTRLDIKESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 155 - protein, 651 aa, synthetic construct
  REGION 1..651: note = anti-BCMA CAR
EVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAKVDGPPSFDIWGQGTMVTVSSGGGGSGGGGSGGGGSSYVLTQPPSVSVAPGQTARITCGANNIGSKSVHWYQQKPGQAPMLVVYDDDDRPSGIPERFSGSNSGNTATLTISGVEAGDEADYFCHLWDRSRDHYVFGTGTKLTVLESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 156 - protein, 658 aa, synthetic construct
  REGION 1..658: note = anti-BCMA CAR
SYELTQPPSASGTPGQRVTMSCSGTSSNIGSHSVNWYQQLPGTAPKLLIYTNNQRPSGVPDRFSGSKSGTSASLAISGLQSEDEADYYCAAWDGSLNGLVFGGGTKLTVLGSRGGGGSGGGGSGGGGSLEMAEVQLVQSGAEVKKPGESLKISCKGSGYSFTSYWIGWVRQMPGKGLEWMGIIYPGDSDTRYSPSFQGHVTISADKSISTAYLQWSSLKASDTAMYYCARYSGSFDNWGQGTLVTVSSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 157 - protein, 654 aa, synthetic construct
  REGION 1..654: note = anti-BCMA CAR
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGSRGGGGSGGGGSGGGGSLEMAEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 158 - protein, 653 aa, synthetic construct
  REGION 1..653: note = anti-BCMA CAR
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGSRGGGGSGGGGSGGGGSLEMAEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 159 - protein, 462 aa, synthetic construct
  REGION 1..462: note = anti-BCMA CAR
EVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSDIQLTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 160 - protein, 469 aa, synthetic construct
  REGION 1..469: note = anti-BCMA CAR
QVQLVESGGGLVQPGRSLRLSCAASGFTFSNYAMSWVRQAPGKGLGWVSGISRSGENTYYADSVKGRFTISRDNSKNTLYLQMNSLRDEDTAVYYCARSPAHYYGGMDVWGQGTTVTVSSASGGGGSGGRASGGGGSDIVLTQSPGTLSLSPGERATLSCRASQSISSSFLAWYQQKPGQAPRLLIYGASRRATGIPDRFSGSGSGTDFTLTISRLEPEDSAVYYCQQYHSSPSWTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 161 - protein, 462 aa, synthetic construct
  REGION 1..462: note = anti-BCMA CAR
QVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSDIRLTQSPSPLSASVGDRVTITCQASEDINKFLNWYHQTPGKAPKLLIYDASTLQTGVPSRFSGSGSGTDFTLTINSLQPEDIGTYYCQQYESLPLTFGGGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 162 - protein, 464 aa, synthetic construct
  REGION 1..464: note = anti-BCMA CAR
EVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSEIVLTQSPGTLSLSPGERATLSCRASQSIGSSSLAWYQQKPGQAPRLLMYGASSRASGIPDRFSGSGSGTDFTLTISRLEPEDFAVYYCQQYAGSPPFTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 163 - protein, 467 aa, synthetic construct
  REGION 1..467: note = anti-BCMA CAR
QIQLVQSGPDLKKPGETVKLSCKASGYTFTNFGMNWVKQAPGKGFKWMAWINTYTGESYFADDFKGRFAFSVETSATTAYLQINNLKTEDTATYFCARGEIYYGYDGGFAYWGQGTLVTVSAGGGGSGGGGSGGGGSDVVMTQSHRFMSTSVGDRVSITCRASQDVNTAVSWYQQKPGQSPKLLIFSASYRYTGVPDRFTGSGSGADFTLTISSVQAEDLAVYYCQQHYSTPWTFGGGTKLDIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 164 - protein, 472 aa, synthetic construct
  REGION 1..472: note = anti-BCMA CAR
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSGGGGSGGGGSQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 165 - protein, 466 aa, synthetic construct
  REGION 1..466: note = anti-BCMA CAR
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSGGGGSGGGGSDIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 166 - protein, 466 aa, synthetic construct
  REGION 1..466: note = anti-BCMA CAR
QIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSSGGGGSGGGGSGGGGSDIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 167 - protein, 466 aa, synthetic construct
  REGION 1..466: note = anti-BCMA CAR
QIQLVQSGPELKKPGETVKISCKASGYTFTHYSMNWVKQAPGKGLKWMGRINTETGEPLYADDFKGRFAFSLETSASTAYLVINNLKNEDTATFFCSNDYLYSCDYWGQGTTLTVSSGGGGSGGGGSGGGGSDIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 168 - protein, 482 aa, synthetic construct
  REGION 1..482: note = anti-BCMA CAR
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 169 - protein, 440 aa, synthetic construct
  REGION 1..440: note = anti-BCMA CAR
QVQLVQSGAEVKKPGASVKVSCKASGYSFPDYYINWVRQAPGQGLEWMGWIYFASGNSEYNQKFTGRVTMTRDTSINTAYMELSSLTSEDTAVYFCASLYDYDWYFDVWGQGTMVTVSSGGGGSGGGGSGGGGSDIVMTQTPLSLSVTPGQPASISCKSSQSLVHSNGNTYLHWYLQKPGQSPQLLIYKVSNRFSGVPDRFSGSGSGTDFTLKISRVEAEDVGIYYCSQSSIYPWTFGQGTKLEIKGLAVSTISSFFPPGYQIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 170 - protein, 469 aa, synthetic construct
  REGION 1..469: note = anti-BCMA CAR
QVQLVQSGAEVKKPGASVKVSCKASGYSFPDYYINWVRQAPGQGLEWMGWIYFASGNSEYNQKFTGRVTMTRDTSINTAYMELSSLTSEDTAVYFCASLYDYDWYFDVWGQGTMVTVSSGGGGSGGGGSGGGGSDIVMTQTPLSLSVTPGQPASISCKSSQSLVHSNGNTYLHWYLQKPGQSPQLLIYKVSNRFSGVPDRFSGSGSGTDFTLKISRVEAEDVGIYYCSQSSIYPWTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 171 - protein, 655 aa, synthetic construct
  REGION 1..655: note = anti-BCMA CAR
QVQLVQSGAEVKKPGASVKVSCKASGYSFPDYYINWVRQAPGQGLEWMGWIYFASGNSEYNQKFTGRVTMTRDTSINTAYMELSSLTSEDTAVYFCASLYDYDWYFDVWGQGTMVTVSSGGGGSGGGGSGGGGSDIVMTQTPLSLSVTPGQPASISCKSSQSLVHSNGNTYLHWYLQKPGQSPQLLIYKVSNRFSGVPDRFSGSGSGTDFTLKISRVEAEDVGIYYCSQSSIYPWTFGQGTKLEIKEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 172 - protein, 440 aa, synthetic construct
  REGION 1..440: note = anti-BCMA CAR
QVQLVQSGAEVKKPGASVKVSCKASGYSFPDYYINWVRQAPGQGLEWMGWIYFASGNSEYNQKFTGRVTMTRDTSSSTAYMELSSLRSEDTAVYFCASLYDYDWYFDVWGQGTMVTVSSGGGGSGGGGSGGGGSDIVMTQTPLSLSVTPGEPASISCKSSQSLVHSNGNTYLHWYLQKPGQSPQLLIYKVSNRFSGVPDRFSGSGSGADFTLKISRVEAEDVGVYYCAETSHVPWTFGQGTKLEIKGLAVSTISSFFPPGYQIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 173 - protein, 469 aa, synthetic construct
  REGION 1..469: note = anti-BCMA CAR
QVQLVQSGAEVKKPGASVKVSCKASGYSFPDYYINWVRQAPGQGLEWMGWIYFASGNSEYNQKFTGRVTMTRDTSSSTAYMELSSLRSEDTAVYFCASLYDYDWYFDVWGQGTMVTVSSGGGGSGGGGSGGGGSDIVMTQTPLSLSVTPGEPASISCKSSQSLVHSNGNTYLHWYLQKPGQSPQLLIYKVSNRFSGVPDRFSGSGSGADFTLKISRVEAEDVGVYYCAETSHVPWTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 174 - protein, 655 aa, synthetic construct
  REGION 1..655: note = anti-BCMA CAR
QVQLVQSGAEVKKPGASVKVSCKASGYSFPDYYINWVRQAPGQGLEWMGWIYFASGNSEYNQKFTGRVTMTRDTSSSTAYMELSSLRSEDTAVYFCASLYDYDWYFDVWGQGTMVTVSSGGGGSGGGGSGGGGSDIVMTQTPLSLSVTPGEPASISCKSSQSLVHSNGNTYLHWYLQKPGQSPQLLIYKVSNRFSGVPDRFSGSGSGADFTLKISRVEAEDVGVYYCAETSHVPWTFGQGTKLEIKEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 175 - protein, 28 aa, synthetic construct
  REGION 1..28: note = CD8a TM
IYIWAPLAGTCGVLLLSLVITLYCNHRN

SEQ ID NO: 176 - protein, 21 aa, synthetic construct
  REGION 1..21: note = CD8a TM
IYIWAPLAGTCGVLLLSLVIT

SEQ ID NO: 177 - protein, 4 aa, synthetic construct
  REGION 1..4: note = Linking Peptide
RAAA

SEQ ID NO: 178 - protein, 118 aa, synthetic construct
  REGION 1..118: note = Variable heavy (VH) Anti-BCMA
EVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAKVDGDYTEDYWGQGTLVTVSS

SEQ ID NO: 179 - protein, 110 aa, synthetic construct
  REGION 1..110: note = Variable light (VL) Anti-BCMA
QSALTQPASVSGSPGQSITISCTGSSSDVGKYNLVSWYQQPPGKAPKLIIYDVNKRPSGVSNRFSGSKSGNTATLTISGLQGDDEADYYCSSYGGSRSYVFGTGTKVTVL

SEQ ID NO: 180 - protein, 118 aa, synthetic construct
  REGION 1..118: note = Variable heavy (VH) Anti-BCMA
EVQLVQSGGGLVQPGRSLRLSCTASGFTFGDYAMSWFRQAPGKGLEWVGFIRSKAYGGTTEYAASVKGRFTISRDDSKSIAYLQMNSLKTEDTAVYYCAAWSAPTDYWGQGTLVTVSS

SEQ ID NO: 181 - protein, 107 aa, synthetic construct
  REGION 1..107: note = Variable light (VL) Anti-BCMA
DIQMTQSPAFLSASVGDRVTVTCRASQGISNYLAWYQQKPGNAPRLLIYSASTLQSGVPSRFRGTGYGTEFSLTIDSLQPEDFATYYCQQSYTSRQTFGPGTRLDIK

SEQ ID NO: 182 - protein, 118 aa, synthetic construct
  REGION 1..118: note = Variable heavy (VH) Anti-BCMA
EVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAKVDGPPSFDIWGQGTMVTVSS

SEQ ID NO: 183 - protein, 108 aa, synthetic construct
  REGION 1..108: note = Variable light (VL) Anti-BCMA
SYVLTQPPSVSVAPGQTARITCGANNIGSKSVHWYQQKPGQAPMLVVYDDDDRPSGIPERFSGSNSGNTATLTISGVEAGDEADYFCHLWDRSRDHYVFGTGTKLTVL

SEQ ID NO: 184 - protein, 122 aa, synthetic construct; identical to SEQ ID NO: 66
  REGION 1..122: note = Variable heavy (VH) Anti-BCMA

SEQ ID NO: 185 - protein, 111 aa, synthetic construct; identical to SEQ ID NO: 67
  REGION 1..111: note = Variable light (VL) Anti-BCMA

SEQ ID NO: 186 - protein, 16 aa, synthetic construct
  REGION 1..16: note = CD33 Signal peptide
MPLLLLLPLLWAGALA

SEQ ID NO: 187 - protein, 18 aa, synthetic construct
  REGION 1..18: note = CD8 alpha signal peptide
MALPVTALLLPLALLLHA

SEQ ID NO: 188 - DNA, 66 bp, synthetic construct
  misc_feature 1..66: note = GMCSFR alpha chain signal sequence
  source 1..66: mol_type = other DNA
atgcttctcctggtgacaagccttctgctctgtgagttaccacacccagcattcctcctgatccca

SEQ ID NO: 189 - protein, 22 aa, synthetic construct
  REGION 1..22: note = GMCSFR alpha chain signal sequence
MLLLVTSLLLCELPHPAFLLIP

SEQ ID NO: 190 - protein, 14 aa, synthetic construct
  REGION 1..14: note = Exemplary IgG Hinge
EVVVKYGPPCPPCP

SEQ ID NO: 191 - skipped

SEQ ID NO: 192 - protein, 15 aa, synthetic construct
  REGION 1..15: note = Exemplary IgG Hinge
EPKSCDKTHTCPPCP

SEQ ID NO: 193 - protein, 12 aa, synthetic construct
  REGION 1..12: note = Exemplary IgG Hinge
ERKCCVECPPCP

SEQ ID NO: 194 - protein, 61 aa, synthetic construct
  REGION 1..61: note = Exemplary IgG Hinge
ELKTPLGDTHTCPRCPEPKSCDTPPPCPRCPEPKSCDTPPPCPRCPEPKSCDTPPPCPRCP

SEQ ID NO: 195 - protein, 12 aa, synthetic construct
  REGION 1..12: note = Exemplary IgG Hinge
ESKYGPPCPSCP

SEQ ID NO: 196 - protein, 12 aa, synthetic construct
  REGION 1..12: note = Exemplary IgG Hinge
ESKYGPPCPPCP

SEQ ID NO: 197 - protein, 9 aa, synthetic construct
  REGION 1..9: note = Exemplary IgG Hinge
YGPPCPPCP

SEQ ID NO: 198 - protein, 10 aa, synthetic construct
  REGION 1..10: note = Exemplary IgG Hinge
KYGPPCPPCP

SEQ ID NO: 199 - protein, 357 aa, synthetic construct
  REGION 1..357: note = tEGFR
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 200 - protein, 18 aa, synthetic construct
  REGION 1..18: note = T2A
EGRGSLLTCGDVEENPGP

SEQ ID NO: 201 - protein, 22 aa, synthetic construct
  REGION 1..22: note = P2A
GSGATNFSLLKQAGDVEENPGP

SEQ ID NO: 202 - protein, 19 aa, synthetic construct
  REGION 1..19: note = P2A
ATNFSLLKQAGDVEENPGP

SEQ ID NO: 203 - protein, 20 aa, synthetic construct
  REGION 1..20: note = E2A
QCTNYALLKLAGDVESNPGP

SEQ ID NO: 204 - protein, 22 aa, synthetic construct
  REGION 1..22: note = F2A
VKQTLNFDLLKLAGDVESNPGP

SEQ ID NO: 205 - protein, 290 aa, synthetic construct
  REGION 1..290: note = BCMA-Fc fusion polypeptide
MLQMAGQCSQNEYFDSLLHACIPCQLRCSSNTPPLTCQRYCNASVTNSVKGTNAGGGGSPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 206 - protein, 5 aa, synthetic construct
  REGION 1..5: note = CDR-H1
DYYVY

SEQ ID NO: 207 - protein, 17 aa, synthetic construct
  REGION 1..17: note = CDR-H2
WINPNSGGTNYAQKFQG

SEQ ID NO: 208 - protein, 9 aa, synthetic construct
  REGION 1..9: note = CDR-H3
SQRDGYMDY

SEQ ID NO: 209 - protein, 7 aa, synthetic construct
  REGION 1..7: note = CDR-H1
GYTFIDY

SEQ ID NO: 210 - protein, 6 aa, synthetic construct
  REGION 1..6: note = CDR-H2
NPNSGG

SEQ ID NO: 211 - protein, 10 aa, synthetic construct
  REGION 1..10: note = CDR-H1
GYTFIDYYVY

SEQ ID NO: 212 - protein, 10 aa, synthetic construct
  REGION 1..10: note = CDR-H2
WINPNSGGTN

SEQ ID NO: 213 - protein, 8 aa, synthetic construct
  REGION 1..8: note = CDR-H1
GYTFIDYY

SEQ ID NO: 214 - protein, 8 aa, synthetic construct
  REGION 1..8: note = CDR-H2
INPNSGGT

SEQ ID NO: 215 - protein, 11 aa, synthetic construct
  REGION 1..11: note = CDR-H3
ARSQRDGYMDY

SEQ ID NO: 216 - protein, 8 aa, synthetic construct
  REGION 1..8: note = CDR-L1
TGTSSDVG

SEQ ID NO: 217 - protein, 7 aa, synthetic construct
  REGION 1..7: note = CDR-L2
EDSKRPS

SEQ ID NO: 218 - protein, 10 aa, synthetic construct
  REGION 1..10: note = CDR-L3
SSNTRSSTLV

SEQ ID NO: 219 - protein, 9 aa, synthetic construct
  REGION 1..9: note = CDR-L1
ISCTGTSSD

SEQ ID NO: 220 - skipped

SEQ ID NO: 221 - protein, 244 aa, synthetic construct
  REGION 1..244: note = anti-BCMA scFv
QSALTQPASVSASPGQSIAISCTGTSSDVGWYQQHPGKAPKLMIYEDSKRPSGVSNRFSGSKSGNTASLTISGLQAEDEADYYCSSNTRSSTLVFGGGTKLTVLGSRGGGGSGGGGSGGGGSLEMAEVQLVQSGAEMKKPGASLKLSCKASGYTFIDYYVYWMRQAPGQGLESMGWINPNSGGTNYAQKFQGRVTMTRDTSISTAYMELSRLRSDDTAMYYCARSQRDGYMDYWGQGTLVTVSS

SEQ ID NO: 222 - protein, 7 aa, synthetic construct
  REGION 1..7: note = CDR-H1
GYTFTDY

SEQ ID NO: 223 - protein, 6 aa, synthetic construct
  REGION 1..6: note = CDR-H2
NTETRE

SEQ ID NO: 224 - protein, 8 aa, synthetic construct
  REGION 1..8: note = CDR-H3
DYSYAMDY

SEQ ID NO: 225 - protein, 15 aa, synthetic construct
  REGION 1..15: note = CDR-L1
RASESVTILGSHLIH

SEQ ID NO: 226 - protein, 7 aa, synthetic construct
  REGION 1..7: note = CDR-L2
LASNVQT

SEQ ID NO: 227 - protein, 9 aa, synthetic construct
  REGION 1..9: note = CDR-L3
LQSRTIPRT

SEQ ID NO: 228 - protein, 10 aa, synthetic construct
  REGION 1..10: note = CDR-H1
GYTFTDYSIN

SEQ ID NO: 229 - protein, 10 aa, synthetic construct
  REGION 1..10: note = CDR-H2
WINTETREPA

SEQ ID NO: 230 - protein, 8 aa, synthetic construct
  REGION 1..8: note = CDR-H3
DYSYAMDY

SEQ ID NO: 231 - protein, 15 aa, synthetic construct
  REGION 1..15: note = CDR-L1
RASESVTILGSHLIH

SEQ ID NO: 232 - protein, 7 aa, synthetic construct
  REGION 1..7: note = CDR-L2
LASNVQT

SEQ ID NO: 233 - protein, 9 aa, synthetic construct
  REGION 1..9: note = CDR-L3
LQSRTIPRT

SEQ ID NO: 234 - protein, 8 aa, synthetic construct
  REGION 1..8: note = CDR-H1
GYTFTDYS

SEQ ID NO: 235 - protein, 8 aa, synthetic construct
  REGION 1..8: note = CDR-H2
INTETREP

SEQ ID NO: 236 - protein, 10 aa, synthetic construct
  REGION 1..10: note = CDR-H3
ALDYSYAMDY

SEQ ID NO: 237 - protein, 10 aa, synthetic construct
  REGION 1..10: note = CDR-L1
ESVTILGSHL

SEQ ID NO: 238 - skipped

SEQ ID NO: 239 - protein, 9 aa, synthetic construct
  REGION 1..9: note = CDR-L3
LQSRTIPRT

SEQ ID NO: 240 - DNA, 1485 bp, synthetic construct; identical to SEQ ID NO: 10
  misc_feature 1..1485: note = BCMA CAR
  source 1..1485: mol_type = other DNA

SEQ ID NO: 241 - protein, 243 aa, synthetic construct
  REGION 1..243: note = Anti-BCMA scFv
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARAEMGAVFDIWGQGTMVTVSSGSTSGSGKPGSGEGSTKGEIVLTQSPATLSLSPGERATLSCRASQSVSRYLAWYQQKPGQAPRLLIYDASNRATGIPARFSGSGSGTDFTLTISSLEPEDFAVYYCQQRISWPFTFGGGTKVEIK

SEQ ID NO: 242 - protein, 244 aa, synthetic construct
  REGION 1..244: note = Anti-BCMA scFv
EIVLTQSPATLSLSPGERATLSCRASQSVSRYLAWYQQKPGQAPRLLIYDASNRATGIPARFSGSGSGTDFTLTISSLEPEDFAVYYCQQRISWPFTFGGGTKVEIKRGSTSGSGKPGSGEGSTKGEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARAEMGAVFDIWGQGTMVTVSS

SEQ ID NO: 243 - protein, 252 aa, synthetic construct
  REGION 1..252: note = Anti-BCMA scFv
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARDGTYLGGLWYFDLWGRGTLVTVSSGSTSGSGKPGSGEGSTKGDIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGLGLPLTFGGGTKVEIK

SEQ ID NO: 244 - protein, 253 aa, synthetic construct
  REGION 1..253: note = Anti-BCMA scFv
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGLGLPLTFGGGTKVEIKRGSTSGSGKPGSGEGSTKGQVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARDGTYLGGLWYFDLWGRGTLVTVSS

SEQ ID NO: 245 - protein, 240 aa, synthetic construct
  REGION 1..240: note = Anti-BCMA scFv
QVQLVQSGAEVKKPGASVKVSCKASGYTFTSYYMHWVRQAPGQGLEWMGIINPGGGSTSYAQKFQGRVTMTRDTSTSTVYMELSSLRSEDTAVYYCARESWPMDVWGQGTTVTVSSGSTSGSGKPGSGEGSTKGEIVMTQSPATLSVSPGERATLSCRASQSVSSNLAWYQQKPGQAPRLLIYGASTRATGIPARFSGSGSGTEFTLTISSLQSEDFAVYYCQQYAAYPTFGGGTKVEIK

SEQ ID NO: 246 - protein, 241 aa, synthetic construct
  REGION 1..241: note = Anti-BCMA scFv
EIVMTQSPATLSVSPGERATLSCRASQSVSSNLAWYQQKPGQAPRLLIYGASTRATGIPARFSGSGSGTEFTLTISSLQSEDFAVYYCQQYAAYPTFGGGTKVEIKRGSTSGSGKPGSGEGSTKGQVQLVQSGAEVKKPGASVKVSCKASGYTFTSYYMHWVRQAPGQGLEWMGIINPGGGSTSYAQKFQGRVTMTRDTSTSTVYMELSSLRSEDTAVYYCARESWPMDVWGQGTTVTVSS

SEQ ID NO: 247 - protein, 247 aa, synthetic construct
  REGION 1..247: note = Anti-BCMA scFv
QLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARGRGYATSLAFDIWGQGTMVTVSSGSTSGSGKPGSGEGSTKGEIVLTQSPATLSLSPGERATLSCRASQSVSSYLAWYQQKPGQAPRLLIYDASNRATGIPARFSGSGSGTDFTLTISSLEPEDFAVYYCQQRHVWPPTFGGGTKVEIK

SEQ ID NO: 248 - protein, 248 aa, synthetic construct
  REGION 1..248: note = Anti-BCMA scFv
EIVLTQSPATLSLSPGERATLSCRASQSVSSYLAWYQQKPGQAPRLLIYDASNRATGIPARFSGSGSGTDFTLTISSLEPEDFAVYYCQQRHVWPPTFGGGTKVEIKRGSTSGSGKPGSGEGSTKGQLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARGRGYATSLAFDIWGQGTMVTVSS

SEQ ID NO: 249 - protein, 244 aa, synthetic construct
  REGION 1..244: note = Anti-BCMA scFv
EVQLVESGGGLVQPGGSLRLSCAASGFTFSSYSMNWVRQAPGKGLEWVSTISSSSSTIYYADSVKGRFTISRDNAKNSLYLQMNSLRAEDTAVYYCARGSQEHLIFDYWGQGTLVTVSSGSTSGSGKPGSGEGSTKGEIVLTQSPATLSLSPGERATLSCRASQSVSRYLAWYQQKPGQAPRLLIYDASNRATGIPARFSGSGSGTDFTLTISSLEPEDFAVYYCQQRFYYPWTFGGGTKVEIK

SEQ ID NO: 250 - protein, 245 aa, synthetic construct
  REGION 1..245: note = Anti-BCMA scFv
EIVLTQSPATLSLSPGERATLSCRASQSVSRYLAWYQQKPGQAPRLLIYDASNRATGIPARFSGSGSGTDFTLTISSLEPEDFAVYYCQQRFYYPWTFGGGTKVEIKRGSTSGSGKPGSGEGSTKGEVQLVESGGGLVQPGGSLRLSCAASGFTFSSYSMNWVRQAPGKGLEWVSTISSSSSTIYYADSVKGRFTISRDNAKNSLYLQMNSLRAEDTAVYYCARGSQEHLIFDYWGQGTLVTVSS

SEQ ID NO: 251 - protein, 247 aa, synthetic construct
  REGION 1..247: note = Anti-BCMA scFv
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARTDFWSGSPPGLDYWGQGTLVTVSSGSTSGSGKPGSGEGSTKGDIQLTQSPSSVSASVGDRVTITCRASQGISSWLAWYQQKPGKAPKLLIYGASSLQSGVPSRFSGSGSGTDFTLTISSLQPEDFATYYCQQIYTFPFTFGGGTKVEIK

SEQ ID NO: 252 - protein, 248 aa, synthetic construct
  REGION 1..248: note = Anti-BCMA scFv
DIQLTQSPSSVSASVGDRVTITCRASQGISSWLAWYQQKPGKAPKLLIYGASSLQSGVPSRFSGSGSGTDFTLTISSLQPEDFATYYCQQIYTFPFTFGGGTKVEIKRGSTSGSGKPGSGEGSTKGQVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARTDFWSGSPPGLDYWGQGTLVTVSS

SEQ ID NO: 253 - protein, 257 aa, synthetic construct
  REGION 1..257: note = Anti-BCMA scFv
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARTPEYSSSIWHYYYGMDVWGQGTTVTVSSGSTSGSGKPGSGEGSTKGDIVMTQSPDSLAVSLGERATINCKSSQSVLYSSNNKNYLAWYQQKPGQPPKLLIYWASTRESGVPDRFSGSGSGTDFTLTISSLQAEDVAVYYCQQFAHTPFTFGGGTKVEIK

SEQ ID NO: 254 - protein, 258 aa, synthetic construct
  REGION 1..258: note = Anti-BCMA scFv
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQFAHTPFTFGGGTKVEIKRGSTSGSGKPGSGEGSTKGQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARTPEYSSSIWHYYYGMDVWGQGTTVTVSS

SEQ ID NO: 255 - protein, 248 aa, synthetic construct
  REGION 1..248: note = Anti-BCMA scFv
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCVKGPLQEPPYDYGMDVWGQGTTVTVSSGSTSGSGKPGSGEGSTKGEIVMTQSPATLSVSPGERATLSCRASQSVSSNLAWYQQKPGQAPRLLIYSASTRATGIPARFSGSGSGTEFTLTISSLQSEDFAVYYCQQHHVWPLTFGGGTKVEIK

SEQ ID NO: 256 - protein, 249 aa, synthetic construct
  REGION 1..249: note = Anti-BCMA scFv
EIVMTQSPATLSVSPGERATLSCRASQSVSSNLAWYQQKPGQAPRLLIYSASTRATGIPARFSGSGSGTEFTLTISSLQSEDFAVYYCQQHHVWPLTFGGGTKVEIKRGSTSGSGKPGSGEGSTKGQVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCVKGPLQEPPYDYGMDVWGQGTTVTVSS

SEQ ID NO: 257 - protein, 246 aa, synthetic construct
  REGION 1..246: note = Anti-BCMA scFv
DIVLTQSPASLAVSLGERATINCRASESVSVIGAHLIHWYQQKPGQPPKLLIYLASNLETGVPARFSGSGSGTDFTLTISSLQAEDAAIYYCLQSRIFPRTFGQGTKLEIKGSTSGSGKPGSGEGSTKGQVQLVQSGSELKKPGASVKVSCKASGYTFTDYSINWVRQAPGQGLEWMGWINTETREPAYAYDFRGRFVFSLDTSVSTAYLQISSLKAEDTAVYYCARDYSYAMDYWGQGTLVTVSS

SEQ ID NO: 258 - protein, 246 aa, synthetic construct; identical to SEQ ID NO: 257
  REGION 1..246: note = Anti-BCMA scFv

SEQ ID NO: 259 - protein, 246 aa, synthetic construct; identical to SEQ ID NO: 257
  REGION 1..246: note = Anti-BCMA scFv

SEQ ID NO: 260 - protein, 239 aa, synthetic construct
  REGION 1..239: note = Anti-BCMA scFv
EVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSDIQLTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKVEIK

SEQ ID NO: 261 - protein, 246 aa, synthetic construct
  REGION 1..246: note = Anti-BCMA scFv
QVQLVESGGGLVQPGRSLRLSCAASGFTFSNYAMSWVRQAPGKGLGWVSGISRSGENTYYADSVKGRFTISRDNSKNTLYLQMNSLRDEDTAVYYCARSPAHYYGGMDVWGQGTTVTVSSASGGGGSGGRASGGGGSDIVLTQSPGTLSLSPGERATLSCRASQSISSSFLAWYQQKPGQAPRLLIYGASRRATGIPDRFSGSGSGTDFTLTISRLEPEDSAVYYCQQYHSSPSWTFGQGTKLEIK

SEQ ID NO: 262 - protein, 239 aa, synthetic construct
  REGION 1..239: note = Anti-BCMA scFv
QVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSDIRLTQSPSPLSASVGDRVTITCQASEDINKFLNWYHQTPGKAPKLLIYDASTLQTGVPSRFSGSGSGTDFTLTINSLQPEDIGTYYCQQYESLPLTFGGGTKVEIK

SEQ ID NO: 263 - protein, 241 aa, synthetic construct
  REGION 1..241: note = Anti-BCMA scFv
EVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGRASGGGGSEIVLTQSPGTLSLSPGERATLSCRASQSIGSSSLAWYQQKPGQAPRLLMYGASSRASGIPDRFSGSGSGTDFTLTISRLEPEDFAVYYCQQYAGSPPFTFGQGTKVEIK

SEQ ID NO: 264 - protein, 243 aa, synthetic construct
  REGION 1..243: note = Anti-BCMA scFv
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSGGGGSGGGGSDIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIK

SEQ ID NO: 265 - protein, 243 aa, synthetic construct
  REGION 1..243: note = Anti-BCMA scFv
QIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSSGGGGSGGGGSGGGGSDIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIK

SEQ ID NO: 266 - protein, 243 aa, synthetic construct
  REGION 1..243: note = Anti-BCMA scFv
QIQLVQSGPELKKPGETVKISCKASGYTFTHYSMNWVKQAPGKGLKWMGRINTETGEPLYADDFKGRFAFSLETSASTAYLVINNLKNEDTATFFCSNDYLYSCDYWGQGTTLTVSSGGGGSGGGGSGGGGSDIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIK

SEQ ID NO: 267 - protein, 246 aa, synthetic construct
  REGION 1..246: note = Anti-BCMA scFv
QVQLVQSGAEVKKPGASVKVSCKASGYSFPDYYINWVRQAPGQGLEWMGWIYFASGNSEYNQKFTGRVTMTRDTSINTAYMELSSLTSEDTAVYFCASLYDYDWYFDVWGQGTMVTVSSGGGGSGGGGSGGGGSDIVMTQTPLSLSVTPGQPASISCKSSQSLVHSNGNTYLHWYLQKPGQSPQLLIYKVSNRFSGVPDRFSGSGSGTDFTLKISRVEAEDVGIYYCSQSSIYPWTFGQGTKLEIK

SEQ ID NO: 268 - protein, 246 aa, synthetic construct; identical to SEQ ID NO: 267
  REGION 1..246: note = Anti-BCMA scFv

SEQ ID NO: 269 - protein, 246 aa, synthetic construct; identical to SEQ ID NO: 267
  REGION 1..246: note = Anti-BCMA scFv

SEQ ID NO: 270 - protein, 246 aa, synthetic construct
  REGION 1..246: note = Anti-BCMA scFv
QVQLVQSGAEVKKPGASVKVSCKASGYSFPDYYINWVRQAPGQGLEWMGWIYFASGNSEYNQKFTGRVTMTRDTSSSTAYMELSSLRSEDTAVYFCASLYDYDWYFDVWGQGTMVTVSSGGGGSGGGGSGGGGSDIVMTQTPLSLSVTPGEPASISCKSSQSLVHSNGNTYLHWYLQKPGQSPQLLIYKVSNRFSGVPDRFSGSGSGADFTLKISRVEAEDVGVYYCAETSHVPWTFGQGTKLEIK

SEQ ID NO: 271 - protein, 246 aa, synthetic construct; identical to SEQ ID NO: 270
  REGION 1..246: note = Anti-BCMA scFv

SEQ ID NO: 272 - protein, 246 aa, synthetic construct; identical to SEQ ID NO: 270
  REGION 1..246: note = Anti-BCMA scFv

SEQ ID NO: 273 - protein, 24 aa, Synthetic construct
  REGION 1..24: note = CD8alpha TM
IYIWAPLAGTCGVLLLSLVITLYC

SEQ ID NO: 274 - skipped

(19) United States
(12) Patent Application Publication
(10) Pub. No.: US 2025/0345432 A1
(43) Pub. Date: Nov. 13, 2025

(54) METHOD FOR PREDICTING RESPONSE TO A T CELL THERAPY

(71) Applicant: Celgene Corporation, Summit, NJ (US)

(72) Inventors: Julie Ann RYTLEWSKI, Seattle, WA (US); Jaymes FULLER, Westfield, NJ (US); David R. MERTZ, Morristown, NJ (US); Timothy CAMPBELL, San Francisco, CA (US)

(73) Assignee: Celgene Corporation, Summit, NJ (US)

(21) Appl. No.: 18/868,674

(22) PCT Filed: May 25, 2023
(86) PCT No.: PCT/US2023/067462

§ 371 Date: Nov. 22, 2024

Related U.S. Application Data
(60) Provisional application No. 63/345,902, filed on May 25, 2022.
(60) Provisional application No. 63/348,982, filed on Jun. 3, 2022.

(51) Int. Cl. A61K 40/42; A61K 40/11; A61K 40/31; A61P 35/00; C07K 14/725; C07K 16/28

(52) U.S. Cl. CPC A61K 40/4215; A61K 40/11; A61K 40/31; A61P 35/00; C07K 14/7051; C07K 16/2878

(57) ABSTRACT

The present disclosure relates to methods for using various markers to predict manufacturing outcomes or clinical responses of subjects, e.g., patients, to administration of a T cell therapy. In some aspects, the T cells of the T cell therapy express recombinant receptors such as chimeric receptors, e.g., chimeric antigen receptors (CARs), or other transgenic receptors, such as T cell receptors (TCRs). Also provided herein are methods for treating subjects, for instance those predicted to exhibit a clinical response.

### CROSS-REFERENCE TO RELATED APPLICATIONS

This application claims priority from U.S. provisional application No. 63/345,902, filed May 25, 2022 and 63/348,982, filed Jun. 3, 2022, both entitled “PREDICTING CLINICAL OUTCOME TO CAR-T CELL THERAPY,” the contents of which are incorporated by reference in their entirety.

### INCORPORATION BY REFERENCE OF SEQUENCE LISTING

The present application is being filed with a Sequence Listing in electronic format. The Sequence Listing is provided as a file entitled 683772002640SeqList.xml, created on May 24, 2023, which is 390,814 bytes in size. The information in electronic format of the Sequence Listing is incorporated by reference in its entirety.

### FIELD

The present disclosure relates to methods for using various markers to predict manufacturing outcomes or clinical responses of subjects, e.g., patients, to administration of a T cell therapy. In some aspects, the T cells of the T cell therapy express recombinant receptors such as chimeric receptors, e.g., chimeric antigen receptors (CARs), or other transgenic receptors, such as T cell receptors (TCRs). Also provided herein are methods for treating subjects, for instance those predicted to exhibit a clinical response.

### BACKGROUND

Various immunotherapy and/or cell therapy methods are available for treating diseases and conditions. For example, adoptive cell therapies (including those involving the administration of cells expressing chimeric receptors specific for a disease or disorder of interest, such as chimeric antigen receptors (CARs) and/or other recombinant antigen receptors, as well as other adoptive immune cell and adoptive T cell therapies) can be beneficial in the treatment of cancer or other diseases or disorders. Improved approaches are needed for determining whether a treatment will result in a beneficial clinical response. Provided herein are methods that address such needs.

### SUMMARY

Provided herein in some embodiments is a method of predicting whether a subject will exhibit a clinical response to a T cell therapy, comprising: (a) obtaining, for a subject having a disease or condition, a parameter of a marker or parameters of a combination of markers, wherein: (i) the parameter or parameters are obtained prior to the subject being administered a T cell therapy comprising T cells comprising a recombinant receptor that binds to an antigen associated with the disease or condition; and (ii) the marker or the combination of markers is selected from the (1) level in a blood sample of d-dimer, (2) level in a blood sample of fibrinogen, (3) level in a blood sample of lymphocytes, (4) level in a blood sample of monocytes, (5) ratio in a blood sample of monocytes to leukocytes, (6) level in a blood sample of red blood cells, (7) level in a blood sample of white blood cells, (8) age, (9) body mass index, (10) level in a blood sample of albumin, (11) level in a blood sample of alkaline phosphatase, (12) level in a blood sample of aspartate aminotransferase, (13) level in a blood sample of alanine aminotransferase, (14) level in a blood sample of direct bilirubin, (15) level in a blood sample of bilirubin, (16) level in a blood sample of creatinine, (17) creatinine clearance, (18) time since diagnosis, (19) number of prior therapies received, (20) time since prior autologous stem cell transplant, (21) time since prior corticosteroid therapy, (22) time since prior alkylating agent therapy, (23) time since prior topoisomerase inhibitor therapy, (24) time since prior proteasome inhibitor therapy, (25) percent in a blood sample of bone marrow plasma cells, (26) level in a blood sample of beta-2 microglobulin, (27) level in a blood sample of Immunoglobulin G, (28) level in a blood sample of lactate dehydrogenase, (29) ratio in a blood sample of kappa to lambda free light chain levels, (30) level in a blood sample of free light chain, (31) level in a blood sample of M-protein, (32) level in a blood sample of platelets, (33) level in a blood sample of sodium, and (34) level in a blood sample of soluble BCMA of the subject; and (b) predicting if the subject is likely to exhibit a clinical response to administration of the T cell therapy for treatment of the disease or condition, wherein the subject is predicted as likely to exhibit the clinical response if: (i) the parameter or one or more of the parameters for markers (3), (6)-(13), (16), (17), (20), (22)-(24), (28), (29), (32), and (33) are higher than an associated threshold level; or (ii) the parameter or one or more of the parameters for markers (1), (2), (4), (5), (14), (15), (18), (19), (21), (25)-(27), (30), (31), and (34) are lower than an associated threshold level.

In some of any embodiments, the subject is predicted as likely to exhibit the clinical response if two or more, three or more, or four or more of any of the criteria of step (b)(i)-(b)(ii) are satisfied.

Also provided herein in some embodiments is a method of predicting whether a subject will not exhibit a clinical response to a T cell therapy, comprising: (a) obtaining, for a subject having a disease or condition, a parameter of a marker or parameters of a combination of markers, wherein: (i) the parameter or parameters are obtained prior to the subject being administered a T cell therapy comprising T cells comprising a recombinant receptor that binds to an antigen associated with the disease or condition; and (ii) the marker or the combination of markers is selected from the (1) level in a blood sample of d-dimer, (2) level in a blood sample of fibrinogen, (3) level in a blood sample of lymphocytes, (4) level in a blood sample of monocytes, (5) ratio in a blood sample of monocytes to leukocytes, (6) level in a blood sample of red blood cells, (7) level in a blood sample of white blood cells, (8) age, (9) body mass index, (10) level in a blood sample of albumin, (11) level in a blood sample of alkaline phosphatase, (12) level in a blood sample of aspartate aminotransferase, (13) level in a blood sample of alanine aminotransferase, (14) level in a blood sample of direct bilirubin, (15) level in a blood sample of bilirubin, (16) level in a blood sample of creatinine, (17) creatinine clearance, (18) time since diagnosis, (19) number of prior therapies received, (20) time since prior autologous stem cell transplant, (21) time since prior corticosteroid therapy, (22) time since prior alkylating agent therapy, (23) time since prior topoisomerase inhibitor therapy, (24) time since prior proteasome inhibitor therapy, (25) percent in a blood sample of bone marrow plasma cells, (26) level in a blood sample of beta-2 microglobulin, (27) level in a blood sample of Immunoglobulin G, (28) level in a blood sample of lactate dehydrogenase, (29) ratio in a blood sample of kappa to lambda free light chain levels, (30) level in a blood sample of free light chain, (31) level in a blood sample of M-protein, (32) level in a blood sample of platelets, (33) level in a blood sample of sodium, and (34) level in a blood sample of soluble BCMA of the subject; and (b) predicting if the subject is likely to not exhibit a clinical response to administration of the T cell therapy for treatment of the disease or condition, wherein the subject is predicted as likely to not exhibit the clinical response if: (i) the parameter or one or more of the parameters for markers (3), (6)-(13), (16), (17), (20), (22)-(24), (28), (29), (32), and (33) are lower than an associated threshold level; or (ii) the parameter or one or more of the parameters for markers (1), (2), (4), (5), (14), (15), (18), (19), (21), (25)-(27), (30), (31), and (34) are higher than an associated threshold level.

In some of any embodiments, the subject is predicted as likely to not exhibit the clinical response if two or more, three or more, or four or more of any of the criteria of step (b)(i)-(b)(ii) are satisfied.

Also provided herein in some embodiments is a method of predicting whether a therapeutically effective T cell therapy can be manufactured for a subject, comprising: (a) obtaining, for a subject having a disease or condition, a parameter of a marker or parameters of a combination of markers, wherein: (i) the parameter or parameters are obtained prior to when T cells for producing an autologous T cell therapy are collected from the subject, the T cell therapy comprising T cells comprising a recombinant receptor that binds to an antigen associated with the disease or condition; and (ii) the marker or the combination of markers is selected from the (1) level in a blood sample of d-dimer, (2) level in a blood sample of fibrinogen, (3) level in a blood sample of lymphocytes, (4) level in a blood sample of monocytes, (5) ratio in a blood sample of monocytes to leukocytes, (6) level in a blood sample of red blood cells, (7) level in a blood sample of white blood cells, (8) age, (9) body mass index, (10) level in a blood sample of albumin, (11) level in a blood sample of alkaline phosphatase, (12) level in a blood sample of aspartate aminotransferase, (13) level in a blood sample of alanine aminotransferase, (14) level in a blood sample of direct bilirubin, (15) level in a blood sample of bilirubin, (16) level in a blood sample of creatinine, (17) creatinine clearance, (18) time since diagnosis, (19) number of prior therapies received, (20) time since prior autologous stem cell transplant, (21) time since prior corticosteroid therapy, (22) time since prior alkylating agent therapy, (23) time since prior topoisomerase inhibitor therapy, (24) time since prior proteasome inhibitor therapy, (25) percent in a blood sample of bone marrow plasma cells, (26) level in a blood sample of beta-2 microglobulin, (27) level in a blood sample of Immunoglobulin G, (28) level in a blood sample of lactate dehydrogenase, (29) ratio in a blood sample of kappa to lambda free light chain levels, (30) level in a blood sample of free light chain, (31) level in a blood sample of M-protein, (32) level in a blood sample of platelets, (33) level in a blood sample of sodium, and (34) level in a blood sample of soluble BCMA of the subject; and (b) predicting if the T cell therapy will be therapeutically effective, wherein the T cell therapy is predicted as likely to be therapeutically effective if: (i) the parameter or one or more of the parameters for markers (3), (6)-(13), (16), (17), (20), (22)-(24), (28), (29), (32), and (33) are higher than an associated threshold level; or (ii) the parameter or one or more of the parameters for markers (1), (2), (4), (5), (14), (15), (18), (19), (21), (25)-(27), (30), (31), and (34) are lower than an associated threshold level.

In some of any embodiments, the T cell therapy is predicted as likely to be therapeutically effective if two or more, three or more, or four or more of any of the criteria of step (b)(i)-(b)(ii) are satisfied.

Also provided herein in some embodiments is a method of predicting whether a therapeutically effective T cell therapy cannot be manufactured for a subject, comprising: (a) obtaining, for a subject having a disease or condition, a parameter of a marker or parameters of a combination of markers, wherein: (i) the parameter or parameters are obtained prior to when T cells for producing an autologous T cell therapy are collected from the subject, the T cell therapy comprising T cells comprising a recombinant receptor that binds to an antigen associated with the disease or condition; and (ii) the marker or the combination of markers is selected from the (1) level in a blood sample of d-dimer, (2) level in a blood sample of fibrinogen, (3) level in a blood sample of lymphocytes, (4) level in a blood sample of monocytes, (5) ratio in a blood sample of monocytes to leukocytes, (6) level in a blood sample of red blood cells, (7) level in a blood sample of white blood cells, (8) age, (9) body mass index, (10) level in a blood sample of albumin, (11) level in a blood sample of alkaline phosphatase, (12) level in a blood sample of aspartate aminotransferase, (13) level in a blood sample of alanine aminotransferase, (14) level in a blood sample of direct bilirubin, (15) level in a blood sample of bilirubin, (16) level in a blood sample of creatinine, (17) creatinine clearance, (18) time since diagnosis, (19) number of prior therapies received, (20) time since prior autologous stem cell transplant, (21) time since prior corticosteroid therapy, (22) time since prior alkylating agent therapy, (23) time since prior topoisomerase inhibitor therapy, (24) time since prior proteasome inhibitor therapy, (25) percent in a blood sample of bone marrow plasma cells, (26) level in a blood sample of beta-2 microglobulin, (27) level in a blood sample of Immunoglobulin G, (28) level in a blood sample of lactate dehydrogenase, (29) ratio in a blood sample of kappa to lambda free light chain levels, (30) level in a blood sample of free light chain, (31) level in a blood sample of M-protein, (32) level in a blood sample of platelets, (33) level in a blood sample of sodium, and (34) level in a blood sample of soluble BCMA of the subject; and (b) predicting if the T cell therapy will not be therapeutically effective, wherein the T cell therapy is predicted as likely to be not therapeutically effective if. (i) the parameter or one or more of the parameters for markers (3), (6)-(13), (16), (17), (20), (22)-(24), (28), (29), (32), and (33) are lower than an associated threshold level; or (ii) the parameter or one or more of the parameters for markers (1), (2), (4), (5), (14), (15), (18), (19), (21), (25)-(27), (30), (31), and (34) are higher than an associated threshold level.

In some of any embodiments, the T cell therapy is predicted as likely to not be therapeutically if two or more, three or more, or four or more of any of the criteria of step (b)(i)-(b)(ii) are satisfied.

In some of any embodiments, the marker is or the combination of markers comprises one or more subject immune profile markers that are selected from markers (1)-(7).

In some of any embodiments, the marker is or the combination of markers comprises one or more subject fitness markers that are selected from markers (8)-(17).

In some of any embodiments, the marker is or the combination of markers comprises one or more subject prior therapy markers that are selected from markers (18)-(24).

In some of any embodiments, the marker is or the combination of markers comprises one or more subject tumor burden markers that are selected from markers (25)-(34).

In some of any embodiments, the combination of markers comprises one or more subject immune profile markers that are selected from markers (1)-(7) and one or more subject tumor burden markers that are selected from markers (25)-(34).

In some of any embodiments, the combination of markers comprises one or more subject immune profile markers that are selected from markers (1)-(7), one or more subject prior therapy markers that are selected from markers (18)-(24), and one or more subject tumor burden markers that are selected from markers (25)-(34).

In some of any embodiments, the threshold level associated with marker (1) is between or between about 0.5 mg/L and 11 mg/L or between or between about 0.5 mg/L and 1.3 mg/L. In some of any embodiments, the threshold level associated with marker (2) is between or between about 2.2 g/L and 7.7 g/L or between or between about 4.2 g/L and 5.4 g/L. In some of any embodiments, the threshold level associated with marker (3) is between or between about 0.3×10⁹ cells/L and 1.0×10⁹ cells/L or between or between about 0.4×10⁹ cells/L and 0.7×10⁹ cells/L. In some of any embodiments, the threshold level associated with marker (4) is between or between about 0.2×10⁹ cells/L and 1.1×10⁹ cells/L or between or between about 0.4×10⁹ cells/L and 0.7×10⁹ cells/L. In some of any embodiments, the threshold level associated with marker (5) is between or between about 6.7 and 18 or between or between about 13 and 14. In some of any embodiments, the threshold level associated with marker (6) is between or between about 2.4×10¹² cells/L and 3.7×10¹² cells/L or between or between about 2.9×10¹² cells/L and 3.3×10¹² cells/L. In some of any embodiments, the threshold level associated with marker (7) is between or between about 2.1×10⁹ cells/L and 7.1×10⁹ cells/L or between or between about 2.9×10⁹ cells/L and 4.2×10⁹ cells/L. In some of any embodiments, the threshold level associated with marker (8) is between or between about 57 years and 66 years or between or between about 64 years and 66 years. In some of any embodiments, the threshold level associated with marker (9) is between or between about 22 kg/m² and 31 kg/m² or between or between about 23 kg/m² and 29 kg/m². In some of any embodiments, the threshold level associated with marker (10) is between or between about 31 g/L and 41 g/L or between or between about 36 g/L and 40 g/L. In some of any embodiments, the threshold level associated with marker (11) is between or between about 28 IU/L and 134 IU/L or between or between about 54 IU/L and 64 IU/L. In some of any embodiments, the threshold level associated with marker (12) is between or between about 7.3 IU/L and 49 IU/L or between or between about 16 IU/L and 26 IU/L. In some of any embodiments, the threshold level associated with marker (13) is between or between about 8 IU/L and 31 IU/L or between or between about 13 IU/L and 29 IU/L. In some of any embodiments, the threshold level associated with marker (14) is between or between about 1.4 μM and 2.7 μM or between or between about 1.8 μM and 2.2 μM. In some of any embodiments, the threshold level associated with marker (15) is between or between about 3.4 μM and 23 μM or between or between about 9.4 μM and 9.6 μM. In some of any embodiments, the threshold level associated with marker (16) is between or between about 46 μM and 114 μM or between or between about 52 μM and 80 μM. In some of any embodiments, the threshold level associated with marker (17) is between or between about 0.8 mL/s and 2.0 mL/s or between or between about 1.9 mL/s and 2.0 mL/s. In some of any embodiments, the threshold level associated with marker (18) is between or between about 2.2 years and 10 years or between or between about 5.5 years and 8.3 years. In some of any embodiments, the threshold level associated with marker (19) is between or between about 4 and 11 or between or between about 4 and 5. In some of any embodiments, the threshold level associated with marker (20) is between or between about 26 days and 3205 days or between or between about 641 days and 2941 days. In some of any embodiments, the threshold level associated with marker (21) is between or between about 12 days and 2257 days or between or between about 42 days and 59 days. In some of any embodiments, the threshold level associated with marker (22) is between or between about 11 days and 493 days or between or between about 230 days and 244 days. In some of any embodiments, the threshold level associated with marker (23) is between or between about 87 days and 3356 days or between or between about 474 days and 676 days. In some of any embodiments, the threshold level associated with marker (24) is between or between about 11 days and 658 days or between or between about 51 days and 170 days. In some of any embodiments, the threshold level associated with marker (25) is between or between about 21% and 100% or between or between about 56% and 80%. In some of any embodiments, the threshold level associated with marker (26) is between or between about 2.7 mg/L and 7.7 mg/L or between or between about 3.2 mg/L and 4.6 mg/L. In some of any embodiments, the threshold level associated with marker (27) is between or between about 2.8 g/L and 75 g/L or between or between about 14 g/L and 35 g/L. In some of any embodiments, the threshold level associated with marker (28) is between or between about 150 IU/L and 319 IU/L or between or between about 181 IU/L and 319 IU/L. In some of any embodiments, the threshold level associated with marker (29) is between or between about 0.003 and 763 or between or between about 8.7 and 211. In some of any embodiments, the threshold level associated with marker (30) is between or between about 0.008 g/L and 12 g/L or between or between about 0.2 g/L and 1.0 g/L. In some of any embodiments, the threshold level associated with marker (31) is between or between about 4.3 g/L and 32 g/L or between or between about 5.3 g/L and 12 g/L. In some of any embodiments, the threshold level associated with marker (32) is between or between about 53×10⁹ cells/L and 212×10⁹ cells/L or between or between about 156×10⁹ cells/L and 181×10⁹ cells/L. In some of any embodiments, the threshold level associated with marker (33) is between or between about 132 mM and 141 mM or between or between about 136 mM and 138 mM. In some of any embodiments, the threshold level associated with marker (34) is between or between about 35 ng/mL and 1300 ng/mL or between or between about 170 ng/mL and 654 ng/mL.

Also provided herein in some embodiments is a method of predicting whether a subject will exhibit a clinical response to a T cell therapy, comprising: (a) obtaining, for a subject having a disease or condition, a parameter of a marker or parameters of a combination of markers, wherein: (i) the parameter or parameters are obtained prior to the subject being administered a T cell therapy comprising T cells comprising a recombinant receptor that binds to an antigen associated with the disease or condition; and (ii) the marker or the combination of markers is selected from any of one or more input composition markers, one or more process markers, one or more drug product markers, one or more subject immune profile markers, one or more subject fitness markers, one or more subject prior therapy markers, and one or more subject tumor burden markers of the subject; and (b) predicting if the subject is likely to exhibit a clinical response to administration of the T cell therapy for treatment of the disease or condition, wherein the predicting comprises comparing the parameter or each of the parameters to an associated threshold level.

Also provided herein in some embodiments is a method of predicting whether a subject will not exhibit a clinical response to a T cell therapy, comprising: (a) obtaining, for a subject having a disease or condition, a parameter of a marker or parameters of a combination of markers, wherein: (i) the parameter or parameters are obtained prior to the subject being administered a T cell therapy comprising T cells comprising a recombinant receptor that binds to an antigen associated with the disease or condition; and (ii) the marker or the combination of markers is selected from any of one or more input composition markers, one or more process markers, one or more drug product markers, one or more subject immune profile markers, one or more subject fitness markers, one or more subject prior therapy markers, and one or more subject tumor burden markers of the subject; and (b) predicting if the subject is likely to not exhibit a clinical response to administration of the T cell therapy for treatment of the disease or condition, wherein the predicting comprises comparing the parameter or each of the parameters to an associated threshold level.

Also provided herein in some embodiments is a method of predicting whether a subject will exhibit a clinical response to a T cell therapy, comprising: (a) obtaining, for a subject having a disease or condition, a parameter of a marker or parameters of a combination of markers, wherein: (i) the parameter or parameters are obtained prior to the subject being administered a T cell therapy comprising T cells comprising a recombinant receptor that binds to an antigen associated with the disease or condition; and (ii) the marker or the combination of markers is selected from any of one or more subject immune profile markers, one or more subject fitness markers, one or more subject prior therapy markers, and one or more subject tumor burden markers of the subject; and (b) predicting if the subject is likely to exhibit a clinical response to administration of the T cell therapy for treatment of the disease or condition, wherein the predicting comprises comparing the parameter or each of the parameters to an associated threshold level.

Also provided herein in some embodiments is a method of predicting whether a subject will not exhibit a clinical response to a T cell therapy, comprising: (a) obtaining, for a subject having a disease or condition, a parameter of a marker or parameters of a combination of markers, wherein: (i) the parameter or parameters are obtained prior to the subject being administered a T cell therapy comprising T cells comprising a recombinant receptor that binds to an antigen associated with the disease or condition; and (ii) the marker or the combination of markers is selected from any of one or more subject immune profile markers, one or more subject fitness markers, one or more subject prior therapy markers, and one or more subject tumor burden markers of the subject; and (b) predicting if the subject is likely to not exhibit a clinical response to administration of the T cell therapy for treatment of the disease or condition, wherein the predicting comprises comparing the parameter or each of the parameters to an associated threshold level.

Also provided herein in some embodiments is a method of predicting whether a therapeutically effective T cell therapy can be manufactured for a subject, comprising: (a) obtaining, for a subject having a disease or condition, a parameter of a marker or parameters of a combination of markers, wherein: (i) the parameter or parameters are obtained prior to when T cells for producing an autologous T cell therapy are collected from the subject, the T cell therapy comprising T cells comprising a recombinant receptor that binds to an antigen associated with the disease or condition; and (ii) the marker or the combination of markers is selected from any of one or more input composition features, one or more process features, one or more drug product features, one or more subject immune profile markers, one or more subject fitness markers, one or more subject prior therapy markers, and one or more subject tumor burden markers of the subject; and (b) predicting if the T cell therapy will be therapeutically effective, wherein the predicting comprises comparing the parameter or each of the parameters to an associated threshold level.

Also provided herein in some embodiments is a method of predicting whether a therapeutically effective T cell therapy can be manufactured for a subject, comprising: (a) obtaining, for a subject having a disease or condition, a parameter of a marker or parameters of a combination of markers, wherein: (i) the parameter or parameters are obtained prior to when T cells for producing an autologous T cell therapy are collected from the subject, the T cell therapy comprising T cells comprising a recombinant receptor that binds to an antigen associated with the disease or condition; and (ii) the marker or the combination of markers is selected from any of one or more subject immune profile markers, one or more subject fitness markers, one or more subject prior therapy markers, and one or more subject tumor burden markers of the subject; and (b) predicting if the T cell therapy will be therapeutically effective, wherein the predicting comprises comparing the parameter or each of the parameters to an associated threshold level.

Also provided herein in some embodiments is a method of predicting whether a therapeutically effective T cell therapy cannot be manufactured for a subject, comprising: (a) obtaining, for a subject having a disease or condition, a parameter of a marker or parameters of a combination of markers, wherein: (i) the parameter or parameters are obtained prior to when T cells for producing an autologous T cell therapy are collected from the subject, the T cell therapy comprising T cells comprising a recombinant receptor that binds to an antigen associated with the disease or condition; and (ii) the marker or the combination of markers is selected from any of one or more input composition features, one or more process features, one or more drug product features, one or more subject immune profile markers, one or more subject fitness markers, one or more subject prior therapy markers, and one or more subject tumor burden markers of the subject; and (b) predicting if the T cell therapy will not be therapeutically effective, wherein the predicting comprises comparing the parameter or each of the parameters to an associated threshold level.

Also provided herein in some embodiments is a method of predicting whether a therapeutically effective T cell therapy cannot be manufactured for a subject, comprising: (a) obtaining, for a subject having a disease or condition, a parameter of a marker or parameters of a combination of markers, wherein: (i) the parameter or parameters are obtained prior to when T cells for producing an autologous T cell therapy are collected from the subject, the T cell therapy comprising T cells comprising a recombinant receptor that binds to an antigen associated with the disease or condition; and (ii) the marker or the combination of markers is selected from any of one or more subject immune profile markers, one or more subject fitness markers, one or more subject prior therapy markers, and one or more subject tumor burden markers of the subject; and (b) predicting if the T cell therapy will not be therapeutically effective, wherein the predicting comprises comparing the parameter or each of the parameters to an associated threshold level.

In some of any embodiments, the parameters of a combination of markers are obtained, and each of the parameters is compared to an associated threshold level.

In some of any embodiments, the combination of markers comprises one or more input composition markers. In some embodiments, the one or more input composition markers are markers determined from cells of an input composition, wherein the input composition comprises peripheral blood mononuclear cells (PBMCs) selected from a biological sample from the subject, wherein T cells from the PBMCs are used for producing the T cell therapy. In some embodiments, the input composition markers are selected from (i) the percentage of CD3+ T cells in the biological sample; (ii) the ratio of CD4+ T cells to CD8+ T cells (CD4:CD8) in the biological sample; (iii) the percentage of CD57+ T cells in the biological sample; and (iv) the percentage of CD28+ T cells in the biological sample.

In some of any embodiments, the subject is predicted as likely to exhibit the clinical response if: (i) the parameter or one or more of the parameters for markers (the percentage of CD3+ T cells in the biological sample), (the ratio of CD4+ T cells to CD8+ T cells (CD4:CD8) in the biological sample), and (the percentage of CD28+ T cells in the biological sample) are higher than an associated threshold level; or (ii) the parameter for marker (the percentage of CD57+ T cells in the biological sample) is lower than an associated threshold level. In some of any embodiments, the subject is predicted as likely to not exhibit the clinical response if: (i) the parameter or one or more of the parameters for markers (the percentage of CD3+ T cells in the biological sample), (the ratio of CD4+ T cells to CD8+ T cells (CD4:CD8) in the biological sample), and (the percentage of CD28+ T cells in the biological sample) are lower than an associated threshold level; or (ii) the parameter for marker (the percentage of CD57+ T cells in the biological sample) is higher than an associated threshold level.

In some of any embodiments, the T cell therapy is predicted as likely to be therapeutically effective if: (i) the parameter or one or more of the parameters for markers (the percentage of CD3+ T cells in the biological sample), (the ratio of CD4+ T cells to CD8+ T cells (CD4:CD8) in the biological sample), and (the percentage of CD28+ T cells in the biological sample) are higher than an associated threshold level; or (ii) the parameter for marker (the percentage of CD57+ T cells in the biological sample) is lower than an associated threshold level. In some of any embodiments, the T cell therapy is predicted as likely to not be therapeutically effective if: (i) the parameter or one or more of the parameters for markers (the percentage of CD3+ T cells in the biological sample), (the ratio of CD4+ T cells to CD8+ T cells (CD4:CD8) in the biological sample), and (the percentage of CD28+ T cells in the biological sample) are lower than an associated threshold level; or (ii) the parameter for marker (the percentage of CD57+ T cells in the biological sample) is higher than an associated threshold level.

In some of any embodiments, the threshold level associated with marker (the percentage of CD3+ T cells in the biological sample) is between or between about 2 percent and 40 percent or between or between about 11 percent and 26 percent. In some of any embodiments, the threshold level associated with marker (the ratio of CD4+ T cells to CD8+ T cells (CD4:CD8) in the biological sample) is between or between about 0.2 and 1.4 or between or between about 0.4 and 0.6. In some of any embodiments, the threshold level associated with marker (the percentage of CD57+ T cells in the biological sample) is between or between about 30 percent and 75 percent or between or between about 45 percent and 65 percent. In some of any embodiments, the threshold level associated with marker (the percentage of CD28+ T cells in the biological sample) is between or between about 50 percent and 90 percent or between or between about 60 percent and 76 percent.

In some of any embodiments, the combination of markers comprises one or more process markers. In some embodiments, the one or more process markers are markers determined from cells of a composition during a process for manufacturing the T cell therapy. In some embodiments, the process for manufacturing the T cell therapy comprises activating T cells of the input composition, introducing the recombinant receptor into the activated T cells, and expanding the T cells to produce the T cell therapy. In some embodiments, the one or more process markers are selected from (i) the size of cells in the composition after activation and (ii) the number of total nucleated cells (TNC) at the end of the process.

In some of any embodiments, the subject is predicted as likely to exhibit the clinical response if: the parameter or one or more of the parameters for markers (the size of cells in the composition after activation) and (the number of total nucleated cells (TNC) at the end of the process) are higher than an associated threshold level. In some of any embodiments, the subject is predicted as likely to not exhibit the clinical response if: the parameter or one or more of the parameters for markers (the size of cells in the composition after activation) and (the number of total nucleated cells (TNC) at the end of the process) are lower than an associated threshold level.

In some of any embodiments, the T cell therapy is predicted as likely to be therapeutically effective if: the parameter or one or more of the parameters for markers (the size of cells in the composition after activation) and (the number of total nucleated cells (TNC) at the end of the process) are higher than an associated threshold level. In some of any embodiments, the T cell therapy is predicted as likely to not be therapeutically effective if: the parameter or one or more of the parameters for markers (the size of cells in the composition after activation) and (the number of total nucleated cells (TNC) at the end of the process) are lower than an associated threshold level.

In some of any embodiments, the threshold level associated with marker (the size of cells in the composition after activation) is between or between about 400 μm³ and 800 μm³ or between or between about 600 μm³ and 780 μm³. In some of any embodiments, the threshold level associated with marker (the number of total nucleated cells (TNC) at the end of the process) is between or between about 1.5×10¹⁰ cells and 7.5×10¹⁰ cells or between or between about 2.6×10¹⁰ cells to 4.5×10¹⁰ cells.

In some of any embodiments, the combination of markers comprises one or more drug product markers. In some embodiments, the one or more drug product markers are markers determined from the cells of the T cell therapy. In some embodiments, the one or more drug product markers are selected from (i) the percentage of cells positive for the recombinant receptor (recombinant receptor+), (ii) the number of total cells positive for the recombinant receptor (total recombinant receptor); (iii) the vector copy number (VCN); and (iv) the level of potency of the T cell therapy. In some embodiments, the one or more drug product markers are selected from (i) the percentage of cells positive for the recombinant receptor (recombinant receptor+), (ii) the number of total cells positive for the recombinant receptor (total recombinant receptor); and (iii) the vector copy number (VCN).

In some of any embodiments, the subject is predicted as likely to exhibit the clinical response if: the parameter or one or more of the parameters for markers (the percentage of cells positive for the recombinant receptor (recombinant receptor+)), (the number of total cells positive for the recombinant receptor (total recombinant receptor)); (the vector copy number (VCN)), and (the level of potency of the T cell therapy) are higher than an associated threshold level. In some of any embodiments, the subject is predicted as likely to not exhibit the clinical response if: the parameter or one or more of the parameters for markers (the percentage of cells positive for the recombinant receptor (recombinant receptor+)), (the number of total cells positive for the recombinant receptor (total recombinant receptor)); (the vector copy number (VCN)), and (the level of potency of the T cell therapy) are lower than an associated threshold level.

In some of any embodiments, the T cell therapy is predicted as likely to be therapeutically effective if: the parameter or one or more of the parameters for markers (the percentage of cells positive for the recombinant receptor (recombinant receptor+)), (the number of total cells positive for the recombinant receptor (total recombinant receptor)); (the vector copy number (VCN)), and (the level of potency of the T cell therapy) are higher than an associated threshold level. In some of any embodiments, the T cell therapy is predicted as likely to not be therapeutically effective if: the parameter or one or more of the parameters for markers (the percentage of cells positive for the recombinant receptor (recombinant receptor+)), (the number of total cells positive for the recombinant receptor (total recombinant receptor)); (the vector copy number (VCN)), and (the level of potency of the T cell therapy) are lower than an associated threshold level.

In some of any embodiments, the threshold level associated with marker (the percentage of cells positive for the recombinant receptor (recombinant receptor+)) is between or between about 30 percent and 55 percent or between or between about 35 percent and 50 percent. In some of any embodiments, the threshold level associated with marker (the number of total cells positive for the recombinant receptor (total recombinant receptor)) is between or between about 0.5×10⁹ cells and 2.0×10⁹ cells or between or between about 1.0×10⁹ cells and 1.75×10⁹ cells. In some of any embodiments, the threshold level associated with marker (the vector copy number (VCN)) is between or between about 4.5 copies/μg DNA and 8.5 copies/μg DNA or between or between about 5 copies/μg DNA and 6.5 copies/μg DNA.

In some of any embodiments, the combination of markers comprises one or more subject immune profile markers. In some of any embodiments, the one or more subject immune profile markers are selected from the (1) level in a blood sample of d-dimer, (2) level in a blood sample of fibrinogen, (3) level in a blood sample of lymphocytes, (4) level in a blood sample of monocytes, (5) ratio in a blood sample of monocytes to leukocytes, (6) level in a blood sample of red blood cells, and (7) level in a blood sample of white blood cells of the subject.

In some of any embodiments, the subject is predicted as likely to exhibit the clinical response if: (i) the parameter or one or more of the parameters for markers (3), (6), and (7) are higher than an associated threshold level; or (ii) the parameter or one or more of the parameters for markers (1), (2), (4), and (5) are lower than an associated threshold level. In some of any embodiments, the subject is predicted as likely to not exhibit the clinical response if: (i) the parameter or one or more of the parameters for markers (3), (6), and (7) are lower than an associated threshold level; or (ii) the parameter or one or more of the parameters for markers (1), (2), (4), and (5) are higher than an associated threshold level.

In some of any embodiments, the T cell therapy is predicted as likely to be therapeutically effective if: (i) the parameter or one or more of the parameters for markers (3), (6), and (7) are higher than an associated threshold level; or (ii) the parameter or one or more of the parameters for markers (1), (2), (4), and (5) are lower than an associated threshold level. In some of any embodiments, the T cell therapy is predicted as likely to not be therapeutically effective if: (i) the parameter or one or more of the parameters for markers (3), (6), and (7) are lower than an associated threshold level; or (ii) the parameter or one or more of the parameters for markers (1), (2), (4), and (5) are higher than an associated threshold level.

In some of any embodiments, the threshold level associated with marker (1) is between or between about 0.5 mg/L and 11 mg/L or between or between about 0.5 mg/L and 1.3 mg/L. In some of any embodiments, the threshold level associated with marker (2) is between or between about 2.2 g/L and 7.7 g/L or between or between about 4.2 g/L and 5.4 g/L. In some of any embodiments, the threshold level associated with marker (3) is between or between about 0.3×10⁹ cells/L and 1.0×10⁹ cells/L or between or between about 0.4×10⁹ cells/L and 0.7×10⁹ cells/L. In some of any embodiments, the threshold level associated with marker (4) is between or between about 0.2×10⁹ cells/L and 1.1×10⁹ cells/L or between or between about 0.4×10⁹ cells/L and 0.7×10⁹ cells/L. In some of any embodiments, the threshold level associated with marker (5) is between or between about 6.7 and 18 or between or between about 13 and 14. In some of any embodiments, the threshold level associated with marker (6) is between or between about 2.4×10¹² cells/L and 3.7×10¹² cells/L or between or between about 2.9×10¹² cells/L and 3.3×10¹² cells/L. In some of any embodiments, the threshold level associated with marker (7) is between or between about 2.1×10⁹ cells/L and 7.1×10⁹ cells/L or between or between about 2.9×10⁹ cells/L and 4.2×10⁹ cells/L.

In some of any embodiments, the combination of markers comprises one or more subject fitness markers. In some of any embodiments, the one or more subject fitness markers are selected from the (8) age, (9) body mass index, (10) level in a blood sample of albumin, (11) level in a blood sample of alkaline phosphatase, (12) level in a blood sample of aspartate aminotransferase, (13) level in a blood sample of alanine aminotransferase, (14) level in a blood sample of direct bilirubin, (15) level in a blood sample of bilirubin, (16) level in a blood sample of creatinine, and (17) creatinine clearance of the subject.

In some of any embodiments, the subject is predicted as likely to exhibit the clinical response if: (i) the parameter or one or more of the parameters for markers (8)-(13), (16), and (17) are higher than an associated threshold level; or (ii) the parameter or one or more of the parameters for markers (14) and (15) are lower than an associated threshold level. In some of any embodiments, the subject is predicted as likely to not exhibit the clinical response if: (i) the parameter or one or more of the parameters for markers (8)-(13), (16), and (17) are lower than an associated threshold level; or (ii) the parameter or one or more of the parameters for markers (14) and (15) are higher than an associated threshold level.

In some of any embodiments, the T cell therapy is predicted as likely to be therapeutically effective if: (i) the parameter or one or more of the parameters for markers (8)-(13), (16), and (17) are higher than an associated threshold level; or (ii) the parameter or one or more of the parameters for markers (14) and (15) are lower than an associated threshold level. In some of any embodiments, the T cell therapy is predicted as likely to not be therapeutically effective if: (i) the parameter or one or more of the parameters for markers (8)-(13), (16), and (17) are lower than an associated threshold level; or (ii) the parameter or one or more of the parameters for markers (14) and (15) are higher than an associated threshold level.

In some of any embodiments, the threshold level associated with marker (8) is between or between about 57 years and 66 years or between or between about 64 years and 66 years. In some of any embodiments, the threshold level associated with marker (9) is between or between about 22 kg/m² and 31 kg/m² or between or between about 23 kg/m² and 29 kg/m². In some of any embodiments, the threshold level associated with marker (10) is between or between about 31 g/L and 41 g/L or between or between about 36 g/L and 40 g/L. In some of any embodiments, the threshold level associated with marker (11) is between or between about 28 IU/L and 134 IU/L or between or between about 54 IU/L and 64 IU/L. In some of any embodiments, the threshold level associated with marker (12) is between or between about 7.3 IU/L and 49 IU/L or between or between about 16 IU/L and 26 IU/L. In some of any embodiments, the threshold level associated with marker (13) is between or between about 8 IU/L and 31 IU/L or between or between about 13 IU/L and 29 IU/L. In some of any embodiments, the threshold level associated with marker (14) is between or between about 1.4 μM and 2.7 μM or between or between about 1.8 μM and 2.2 μM. In some of any embodiments, the threshold level associated with marker (15) is between or between about 3.4 μM and 23 μM or between or between about 9.4 μM and 9.6 μM. In some of any embodiments, the threshold level associated with marker (16) is between or between about 46 μM and 114 μM or between or between about 52 μM and 80 μM. In some of any embodiments, the threshold level associated with marker (17) is between or between about 0.8 mL/s and 2.0 mL/s or between or between about 1.9 mL/s and 2.0 mL/s.

In some of any embodiments, the combination of markers comprises one or more subject prior therapy markers. In some of any embodiments, the one or more subject prior therapy markers are selected from the (18) time since diagnosis, (19) number of prior therapies received, (20) time since prior autologous stem cell transplant, (21) time since prior corticosteroid therapy, (22) time since prior alkylating agent therapy, (23) time since prior topoisomerase inhibitor therapy, and (24) time since prior proteasome inhibitor therapy for the subject.

In some of any embodiments, the subject is predicted as likely to exhibit the clinical response if: (i) the parameter or one or more of the parameters for markers (20) and (22)-(24) are higher than an associated threshold level; or (ii) the parameter or one or more of the parameters for markers (18), (19), and (21) are lower than an associated threshold level. In some of any embodiments, the subject is predicted as likely to not exhibit the clinical response if: (i) the parameter or one or more of the parameters for markers (20) and (22)-(24) are lower than an associated threshold level; or (ii) the parameter or one or more of the parameters for markers (18), (19), and (21) are higher than an associated threshold level.

In some of any embodiments, the T cell therapy is predicted as likely to exhibit be therapeutically effective if: (i) the parameter or one or more of the parameters for markers (20) and (22)-(24) are higher than an associated threshold level; or (ii) the parameter or one or more of the parameters for markers (18), (19), and (21) are lower than an associated threshold level. In some of any embodiments, the T cell therapy is predicted as likely to not be therapeutically effective if: (i) the parameter or one or more of the parameters for markers (20) and (22)-(24) are lower than an associated threshold level; or (ii) the parameter or one or more of the parameters for markers (18), (19), and (21) are higher than an associated threshold level.

In some of any embodiments, the threshold level associated with marker (18) is between or between about 2.2 years and 10 years or between or between about 5.5 years and 8.3 years. In some of any embodiments, the threshold level associated with marker (19) is between or between about 4 and 11 or between or between about 4 and 5. In some of any embodiments, the threshold level associated with marker (20) is between or between about 26 days and 3205 days or between or between about 641 days and 2941 days. In some of any embodiments, the threshold level associated with marker (21) is between or between about 12 days and 2257 days or between or between about 42 days and 59 days. In some of any embodiments, the threshold level associated with marker (22) is between or between about 11 days and 493 days or between or between about 230 days and 244 days. In some of any embodiments, the threshold level associated with marker (23) is between or between about 87 days and 3356 days or between or between about 474 days and 676 days. In some of any embodiments, the threshold level associated with marker (24) is between or between about 11 days and 658 days or between or between about 51 days and 170 days.

In some of any embodiments, the combination of markers comprises one or more subject tumor burden markers. In some of any embodiments, the one or more subject tumor burden markers are selected from the (25) percent in a blood sample of bone marrow plasma cells, (26) level in a blood sample of beta-2 microglobulin, (27) level in a blood sample of Immunoglobulin G, (28) level in a blood sample of lactate dehydrogenase, (29) ratio in a blood sample of kappa to lambda free light chain levels, (30) level in a blood sample of free light chain, (31) level in a blood sample of M-protein, (32) level in a blood sample of platelets, (33) level in a blood sample of sodium, and (34) level in a blood sample of soluble BCMA of the subject.

In some of any embodiments, the subject is predicted as likely to exhibit the clinical response if: (i) the parameter or one or more of the parameters for markers (28), (29), (32), and (33) are higher than an associated threshold level; or (ii) the parameter or one or more of the parameters for markers (25)-(27), (30), (31), and (34) are lower than an associated threshold level. In some of any embodiments, the subject is predicted as likely to not exhibit the clinical response if: (i) the parameter or one or more of the parameters for markers (28), (29), (32), and (33) are lower than an associated threshold level; or (ii) the parameter or one or more of the parameters for markers (25)-(27), (30), (31), and (34) are higher than an associated threshold level.

In some of any embodiments, the T cell therapy is predicted as likely to be therapeutically effective if: (i) the parameter or one or more of the parameters for markers (28), (29), (32), and (33) are higher than an associated threshold level; or (ii) the parameter or one or more of the parameters for markers (25)-(27), (30), (31), and (34) are lower than an associated threshold level. In some of any embodiments, the T cell therapy is predicted as likely to not be therapeutically effective if: (i) the parameter or one or more of the parameters for markers (28), (29), (32), and (33) are lower than an associated threshold level; or (ii) the parameter or one or more of the parameters for markers (25)-(27), (30), (31), and (34) are higher than an associated threshold level.

In some of any embodiments, the threshold level associated with marker (25) is between or between about 21% and 100% or between or between about 56% and 80%. In some of any embodiments, the threshold level associated with marker (26) is between or between about 2.7 mg/L and 7.7 mg/L or between or between about 3.2 mg/L and 4.6 mg/L. In some of any embodiments, the threshold level associated with marker (27) is between or between about 2.8 g/L and 75 g/L or between or between about 14 g/L and 35 g/L. In some of any embodiments, the threshold level associated with marker (28) is between or between about 150 IU/L and 319 IU/L or between or between about 181 IU/L and 319 IU/L. In some of any embodiments, the threshold level associated with marker (29) is between or between about 0.003 and 763 or between or between about 8.7 and 211. In some of any embodiments, the threshold level associated with marker (30) is between or between about 0.008 g/L and 12 g/L or between or between about 0.2 g/L and 1.0 g/L. In some of any embodiments, the threshold level associated with marker (31) is between or between about 4.3 g/L and 32 g/L or between or between about 5.3 g/L and 12 g/L. In some of any embodiments, the threshold level associated with marker (32) is between or between about 53×10⁹ cells/L and 212×10⁹ cells/L or between or between about 156×10⁹ cells/L and 181×10⁹ cells/L. In some of any embodiments, the threshold level associated with marker (33) is between or between about 132 mM and 141 mM or between or between about 136 mM and 138 mM. In some of any embodiments, the threshold level associated with marker (34) is between or between about 35 ng/mL and 1300 ng/mL or between or between about 170 ng/mL and 654 ng/mL.

In some of any embodiments, the combination of markers comprises one or more subject immune profile markers and one or more subject tumor burden markers. In some of any embodiments, the combination of markers comprises one or more subject immune profile markers that are selected from markers (1)-(7) and one or more subject tumor burden markers that are selected from markers (25)-(34).

In some of any embodiments, the combination of markers comprises one or more subject immune profile markers, one or more subject prior therapy markers, and one or more subject tumor burden markers. In some of any embodiments, the combination of markers comprises one or more subject immune profile markers that are selected from markers (1)-(7), one or more subject prior therapy markers that are selected from markers (18)-(24), and one or more subject tumor burden markers that are selected from markers (25)-(34).

In some of any embodiments, the combination of markers comprises the (3) level in a blood sample of lymphocytes, (22) time since prior alkylating agent therapy, and (26) level in a blood sample of beta-2 microglobulin of the subject.

In some of any embodiments, the subject is predicted as likely to exhibit the clinical response if: (i) the parameter for marker (3) is higher than an associated threshold level; (ii) the parameter for marker (22) is higher than an associated threshold level; or (iii) the parameter for marker (26) is lower than an associated threshold level. In some of any embodiments, the subject is predicted as likely to exhibit the clinical response if: (i) the parameter for marker (3) is higher than an associated threshold level; (ii) the parameter for marker (22) is higher than an associated threshold level; and (iii) the parameter for marker (26) is lower than an associated threshold level.

In some of any embodiments, the subject is predicted as likely to not exhibit the clinical response if: (i) the parameter for marker (3) is lower than an associated threshold level; (ii) the parameter for marker (22) is lower than an associated threshold level; or (iii) the parameter for marker (26) is higher than an associated threshold level. In some of any embodiments, the subject is predicted as likely to not exhibit the clinical response if: (i) the parameter for marker (3) is lower than an associated threshold level; (ii) the parameter for marker (22) is lower than an associated threshold level; and (iii) the parameter for marker (26) is higher than an associated threshold level.

In some of any embodiments, the T cell therapy is predicted as likely to be therapeutically effective if: (i) the parameter for marker (3) is higher than an associated threshold level; (ii) the parameter for marker (22) is higher than an associated threshold level; or (iii) the parameter for marker (26) is lower than an associated threshold level. In some of any embodiments, the T cell therapy is predicted as likely to be therapeutically effective if: (i) the parameter for marker (3) is higher than an associated threshold level; (ii) the parameter for marker (22) is higher than an associated threshold level; and (iii) the parameter for marker (26) is lower than an associated threshold level.

In some of any embodiments, the T cell therapy is predicted as likely to not be therapeutically effective if: (i) the parameter for marker (3) is lower than an associated threshold level; (ii) the parameter for marker (22) is lower than an associated threshold level; or (iii) the parameter for marker (26) is higher than an associated threshold level. In some of any embodiments, the T cell therapy is predicted as likely to not be therapeutically effective if: (i) the parameter for marker (3) is lower than an associated threshold level; (ii) the parameter for marker (22) is lower than an associated threshold level; and (iii) the parameter for marker (26) is higher than an associated threshold level.

In some of any embodiments, the threshold level associated with marker (3) is between or between about 0.3×10⁹ cells/L and 1.0×10⁹ cells/L or between or between about 0.4×10⁹ cells/L and 0.7×10⁹ cells/L. In some of any embodiments, the threshold level associated with marker (22) is between or between about 11 days and 493 days or between or between about 230 days and 244 days. In some of any embodiments, the threshold level associated with marker (26) is between or between about 2.7 mg/L and 7.7 mg/L or between or between about 3.2 mg/L and 4.6 mg/L.

In some of any embodiments, the combination of markers comprise the (5) ratio in a blood sample of monocytes to leukocytes, (24) time since prior proteasome inhibitor therapy, (28) level in a blood sample of lactate dehydrogenase, and (31) level in a blood sample of M-protein of the subject.

In some of any embodiments, the subject is predicted as likely to exhibit the clinical response if: (i) the parameter for marker (5) is lower than an associated threshold level; (ii) the parameter for marker (24) is higher than an associated threshold level; (iii) the parameter for marker (28) is higher than an associated threshold level; or (iv) the parameter for marker (31) is lower than an associated threshold level. In some of any embodiments, the subject is predicted as likely to exhibit the clinical response if: (i) the parameter for marker (5) is lower than an associated threshold level; (ii) the parameter for marker (24) is higher than an associated threshold level; (iii) the parameter for marker (28) is higher than an associated threshold level; and (iv) the parameter for marker (31) is lower than an associated threshold level.

In some of any embodiments, the subject is predicted as likely to not exhibit the clinical response if: (i) the parameter for marker (5) is higher than an associated threshold level; (ii) the parameter for marker (24) is lower than an associated threshold level; (iii) the parameter for marker (28) is lower than an associated threshold level; or (iv) the parameter for marker (31) is higher than an associated threshold level. In some of any embodiments, the subject is predicted as likely to not exhibit the clinical response if: (i) the parameter for marker (5) is higher than an associated threshold level; (ii) the parameter for marker (24) is lower than an associated threshold level; (iii) the parameter for marker (28) is lower than an associated threshold level; and (iv) the parameter for marker (31) is higher than an associated threshold level.

In some of any embodiments, the T cell therapy is predicted as likely to be therapeutically effective if: (i) the parameter for marker (5) is lower than an associated threshold level; (ii) the parameter for marker (24) is higher than an associated threshold level; (iii) the parameter for marker (28) is higher than an associated threshold level; or (iv) the parameter for marker (31) is lower than an associated threshold level. In some of any embodiments, the T cell therapy is predicted as likely to be therapeutically effective if: (i) the parameter for marker (5) is lower than an associated threshold level; (ii) the parameter for marker (24) is higher than an associated threshold level; (iii) the parameter for marker (28) is higher than an associated threshold level; and (iv) the parameter for marker (31) is lower than an associated threshold level.

In some of any embodiments, the T cell therapy is predicted as likely to not be therapeutically effective if: (i) the parameter for marker (5) is higher than an associated threshold level; (ii) the parameter for marker (24) is lower than an associated threshold level; (iii) the parameter for marker (28) is lower than an associated threshold level; or (iv) the parameter for marker (31) is higher than an associated threshold level. In some of any embodiments, the T cell therapy is predicted as likely to not be therapeutically effective if: (i) the parameter for marker (5) is higher than an associated threshold level; (ii) the parameter for marker (24) is lower than an associated threshold level; (iii) the parameter for marker (28) is lower than an associated threshold level; and (iv) the parameter for marker (31) is higher than an associated threshold level.

In some of any embodiments, the threshold level associated with marker (5) is between or between about 6.7 and 18 or between or between about 13 and 14. In some of any embodiments, the threshold level associated with marker (24) is between or between about 11 days and 658 days or between or between about 51 days and 170 days. In some of any embodiments, the threshold level associated with marker (28) is between or between about 150 IU/L and 319 IU/L or between or between about 181 IU/L and 319 IU/L. In some of any embodiments, the threshold level associated with marker (31) is between or between about 4.3 g/L and 32 g/L or between or between about 5.3 g/L and 12 g/L.

Also provided herein in some embodiments is a method of predicting whether a subject will exhibit a clinical response to a T cell therapy, comprising: (a) obtaining, for a subject having a disease or condition, a parameter of a marker or parameters of a combination of markers, wherein: (i) the parameter or parameters are obtained prior to the subject being administered a T cell therapy comprising T cells comprising a recombinant receptor that binds to an antigen associated with the disease or condition; and (ii) the marker or the combination of markers is selected from the (1) level in a blood sample of d-dimer, (2) level in a blood sample of fibrinogen, (3) level in a blood sample of lymphocytes, (4) level in a blood sample of monocytes, (5) ratio in a blood sample of monocytes to leukocytes, (6) level in a blood sample of red blood cells, (7) level in a blood sample of white blood cells, (8) age, (9) body mass index, (10) level in a blood sample of albumin, (11) level in a blood sample of alkaline phosphatase, (12) level in a blood sample of aspartate aminotransferase, (13) level in a blood sample of alanine aminotransferase, (14) level in a blood sample of direct bilirubin, (15) level in a blood sample of bilirubin, (16) level in a blood sample of creatinine, (17) creatinine clearance, (18) time since diagnosis, (19) number of prior therapies received, (20) time since prior autologous stem cell transplant, (21) time since prior corticosteroid therapy, (22) time since prior alkylating agent therapy, (23) time since prior topoisomerase inhibitor therapy, (24) time since prior proteasome inhibitor therapy, (25) percent in a blood sample of bone marrow plasma cells, (26) level in a blood sample of beta-2 microglobulin, (27) level in a blood sample of Immunoglobulin G, (28) level in a blood sample of lactate dehydrogenase, (29) ratio in a blood sample of kappa to lambda free light chain levels, (30) level in a blood sample of free light chain, (31) level in a blood sample of M-protein, (32) level in a blood sample of platelets, (33) level in a blood sample of sodium, and (34) level in a blood sample of soluble BCMA of the subject; and (b) predicting if the subject is likely to exhibit a clinical response to administration of the T cell therapy for treatment of the disease or condition based on one or more outputs of a process configured to predict, based on the marker or combination of markers, if the subject is likely to exhibit the clinical response, wherein the predicting comprises providing the parameter or parameters as input to the process.

Also provided herein in some embodiments is a method of predicting whether a subject will not exhibit a clinical response to a T cell therapy, comprising: (a) obtaining, for a subject having a disease or condition, a parameter of a marker or parameters of a combination of markers, wherein: (i) the parameter or parameters are obtained prior to the subject being administered a T cell therapy comprising T cells comprising a recombinant receptor that binds to an antigen associated with the disease or condition; and (ii) the marker or the combination of markers is selected from the (1) level in a blood sample of d-dimer, (2) level in a blood sample of fibrinogen, (3) level in a blood sample of lymphocytes, (4) level in a blood sample of monocytes, (5) ratio in a blood sample of monocytes to leukocytes, (6) level in a blood sample of red blood cells, (7) level in a blood sample of white blood cells, (8) age, (9) body mass index, (10) level in a blood sample of albumin, (11) level in a blood sample of alkaline phosphatase, (12) level in a blood sample of aspartate aminotransferase, (13) level in a blood sample of alanine aminotransferase, (14) level in a blood sample of direct bilirubin, (15) level in a blood sample of bilirubin, (16) level in a blood sample of creatinine, (17) creatinine clearance, (18) time since diagnosis, (19) number of prior therapies received, (20) time since prior autologous stem cell transplant, (21) time since prior corticosteroid therapy, (22) time since prior alkylating agent therapy, (23) time since prior topoisomerase inhibitor therapy, (24) time since prior proteasome inhibitor therapy, (25) percent in a blood sample of bone marrow plasma cells, (26) level in a blood sample of beta-2 microglobulin, (27) level in a blood sample of Immunoglobulin G, (28) level in a blood sample of lactate dehydrogenase, (29) ratio in a blood sample of kappa to lambda free light chain levels, (30) level in a blood sample of free light chain, (31) level in a blood sample of M-protein, (32) level in a blood sample of platelets, (33) level in a blood sample of sodium, and (34) level in a blood sample of soluble BCMA of the subject; and (b) predicting if the subject is likely to not exhibit a clinical response to administration of the T cell therapy for treatment of the disease or condition based on one or more outputs of a process configured to predict, based on the marker or combination of markers, if the subject is likely to not exhibit the clinical response, wherein the predicting comprises providing the parameter or parameters as input to the process.

Also provided herein in some embodiments is a method of predicting whether a therapeutically effective T cell therapy can be manufactured for a subject, comprising: (a) obtaining, for a subject having a disease or condition, a parameter of a marker or parameters of a combination of markers, wherein: (i) the parameter or parameters are obtained prior to when T cells for producing an autologous T cell therapy are collected from the subject, the T cell therapy comprising T cells comprising a recombinant receptor that binds to an antigen associated with the disease or condition; and (ii) the marker or the combination of markers is selected from the (1) level in a blood sample of d-dimer, (2) level in a blood sample of fibrinogen, (3) level in a blood sample of lymphocytes, (4) level in a blood sample of monocytes, (5) ratio in a blood sample of monocytes to leukocytes, (6) level in a blood sample of red blood cells, (7) level in a blood sample of white blood cells, (8) age, (9) body mass index, (10) level in a blood sample of albumin, (11) level in a blood sample of alkaline phosphatase, (12) level in a blood sample of aspartate aminotransferase, (13) level in a blood sample of alanine aminotransferase, (14) level in a blood sample of direct bilirubin, (15) level in a blood sample of bilirubin, (16) level in a blood sample of creatinine, (17) creatinine clearance, (18) time since diagnosis, (19) number of prior therapies received, (20) time since prior autologous stem cell transplant, (21) time since prior corticosteroid therapy, (22) time since prior alkylating agent therapy, (23) time since prior topoisomerase inhibitor therapy, (24) time since prior proteasome inhibitor therapy, (25) percent in a blood sample of bone marrow plasma cells, (26) level in a blood sample of beta-2 microglobulin, (27) level in a blood sample of Immunoglobulin G, (28) level in a blood sample of lactate dehydrogenase, (29) ratio in a blood sample of kappa to lambda free light chain levels, (30) level in a blood sample of free light chain, (31) level in a blood sample of M-protein, (32) level in a blood sample of platelets, (33) level in a blood sample of sodium, and (34) level in a blood sample of soluble BCMA of the subject; and (b) predicting if the T cell therapy will be therapeutically effective based on one or more outputs of a process configured to predict, based on the marker or combination of markers, if the T cell therapy will be therapeutically effective, wherein the predicting comprises providing the parameter or parameters as input to the process.

Also provided herein in some embodiments is a method of predicting whether a therapeutically effective T cell therapy cannot be manufactured for a subject, comprising: (a) obtaining, for a subject having a disease or condition, a parameter of a marker or parameters of a combination of markers, wherein: (i) the parameter or parameters are obtained prior to when T cells for producing an autologous T cell therapy are collected from the subject, the T cell therapy comprising T cells comprising a recombinant receptor that binds to an antigen associated with the disease or condition; and (ii) the marker or the combination of markers is selected from the (1) level in a blood sample of d-dimer, (2) level in a blood sample of fibrinogen, (3) level in a blood sample of lymphocytes, (4) level in a blood sample of monocytes, (5) ratio in a blood sample of monocytes to leukocytes, (6) level in a blood sample of red blood cells, (7) level in a blood sample of white blood cells, (8) age, (9) body mass index, (10) level in a blood sample of albumin, (11) level in a blood sample of alkaline phosphatase, (12) level in a blood sample of aspartate aminotransferase, (13) level in a blood sample of alanine aminotransferase, (14) level in a blood sample of direct bilirubin, (15) level in a blood sample of bilirubin, (16) level in a blood sample of creatinine, (17) creatinine clearance, (18) time since diagnosis, (19) number of prior therapies received, (20) time since prior autologous stem cell transplant, (21) time since prior corticosteroid therapy, (22) time since prior alkylating agent therapy, (23) time since prior topoisomerase inhibitor therapy, (24) time since prior proteasome inhibitor therapy, (25) percent in a blood sample of bone marrow plasma cells, (26) level in a blood sample of beta-2 microglobulin, (27) level in a blood sample of Immunoglobulin G, (28) level in a blood sample of lactate dehydrogenase, (29) ratio in a blood sample of kappa to lambda free light chain levels, (30) level in a blood sample of free light chain, (31) level in a blood sample of M-protein, (32) level in a blood sample of platelets, (33) level in a blood sample of sodium, and (34) level in a blood sample of soluble BCMA of the subject; and (b) predicting if the T cell therapy will not be therapeutically effective based on one or more outputs of a process configured to predict, based on the marker or combination of markers, if the T cell therapy will not be therapeutically effective, wherein the predicting comprises providing the parameter or parameters as input to the process.

In some of any embodiments, the parameters of a combination of markers are obtained and provided as input to the process.

In some of any embodiments, the marker is or the combination of markers comprises one or more subject immune profile markers that are selected from markers (1)-(7).

In some of any embodiments, the marker is or the combination of markers comprises one or more subject fitness markers that are selected from markers (8)-(17).

In some of any embodiments, the marker is or the combination of markers comprises one or more subject prior therapy markers that are selected from markers (18)-(24).

In some of any embodiments, the marker is or the combination of markers comprises one or more subject tumor burden markers that are selected from markers (25)-(34).

In some of any embodiments, the combination of markers comprises one or more subject immune profile markers that are selected from markers (1)-(7) and one or more subject tumor burden markers that are selected from markers (25)-(34).

In some of any embodiments, the combination of markers comprises one or more subject immune profile markers that are selected from markers (1)-(7), one or more subject prior therapy markers that are selected from markers (18)-(24), and one or more subject tumor burden markers that are selected from markers (25)-(34).

Also provided herein in some embodiments is a method of predicting whether a subject will exhibit a clinical response to a T cell therapy, comprising: (a) obtaining, for a subject having a disease or condition, a parameter of a marker or parameters of a combination of markers, wherein: (i) the parameter or parameters are obtained prior to the subject being administered a T cell therapy comprising T cells comprising a recombinant receptor that binds to an antigen associated with the disease or condition; and (ii) the marker or the combination of markers is selected from any of one or more input composition markers, one or more process markers, one or more drug product markers, one or more subject immune profile markers, one or more subject fitness markers, one or more subject prior therapy markers, and one or more subject tumor burden markers of the subject; and (b) predicting if the subject is likely to exhibit a clinical response to administration of the T cell therapy for treatment of the disease or condition based on one or more outputs of a process configured to predict, based on the marker or combination of markers, if the subject is likely to exhibit the clinical response, wherein the predicting comprises providing the parameter or parameters as input to the process.

Also provided herein in some embodiments is a method of predicting whether a subject will not exhibit a clinical response to a T cell therapy, comprising: (a) obtaining, for a subject having a disease or condition, a parameter of a marker or parameters of a combination of markers, wherein: (i) the parameter or parameters are obtained prior to the subject being administered a T cell therapy comprising T cells comprising a recombinant receptor that binds to an antigen associated with the disease or condition; and (ii) the marker or the combination of markers is selected from any of one or more input composition markers, one or more process markers, one or more drug product markers, one or more subject immune profile markers, one or more subject fitness markers, one or more subject prior therapy markers, and one or more subject tumor burden markers of the subject; and (b) predicting if the subject is likely to not exhibit a clinical response to administration of the T cell therapy for treatment of the disease or condition based on one or more outputs of a process configured to predict, based on the marker or combination of markers, if the subject is likely to not exhibit the clinical response, wherein the predicting comprises providing the parameter or parameters as input to the process.

Also provided herein in some embodiments is a method of predicting whether a subject will exhibit a clinical response to a T cell therapy, comprising: (a) obtaining, for a subject having a disease or condition, a parameter of a marker or parameters of a combination of markers, wherein: (i) the parameter or parameters are obtained prior to the subject being administered a T cell therapy comprising T cells comprising a recombinant receptor that binds to an antigen associated with the disease or condition; and (ii) the marker or the combination of markers is selected from any of one or more subject immune profile markers, one or more subject fitness markers, one or more subject prior therapy markers, and one or more subject tumor burden markers of the subject; and (b) predicting if the subject is likely to exhibit a clinical response to administration of the T cell therapy for treatment of the disease or condition based on one or more outputs of a process configured to predict, based on the marker or combination of markers, if the subject is likely to exhibit the clinical response, wherein the predicting comprises providing the parameter or parameters as input to the process.

Also provided herein in some embodiments is a method of predicting whether a subject will not exhibit a clinical response to a T cell therapy, comprising:(a) obtaining, for a subject having a disease or condition, a parameter of a marker or parameters of a combination of markers, wherein: (i) the parameter or parameters are obtained prior to the subject being administered a T cell therapy comprising T cells comprising a recombinant receptor that binds to an antigen associated with the disease or condition; and (ii) the marker or the combination of markers is selected from any of one or more subject immune profile markers, one or more subject fitness markers, one or more subject prior therapy markers, and one or more subject tumor burden markers of the subject; and (b) predicting if the subject is likely to not exhibit a clinical response to administration of the T cell therapy for treatment of the disease or condition based on one or more outputs of a process configured to predict, based on the marker or combination of markers, if the subject is likely to not exhibit the clinical response, wherein the predicting comprises providing the parameter or parameters as input to the process.

Also provided herein in some embodiments is a method of predicting whether a therapeutically effective T cell therapy can be manufactured for a subject, comprising: (a) obtaining, for a subject having a disease or condition, a parameter of a marker or parameters of a combination of markers, wherein: (i) the parameter or parameters are obtained prior to when T cells for producing an autologous T cell therapy are collected from the subject, the T cell therapy comprising T cells comprising a recombinant receptor that binds to an antigen associated with the disease or condition; and (ii) the marker or the combination of markers is selected from any of one or more input composition markers, one or more process markers, one or more drug product markers, one or more subject immune profile markers, one or more subject fitness markers, one or more subject prior therapy markers, and one or more subject tumor burden markers of the subject; and (b) predicting if the T cell therapy will be therapeutically effective based on one or more outputs of a process configured to predict, based on the marker or combination of markers, if the T cell therapy will be therapeutically effective, wherein the predicting comprises providing the parameter or parameters as input to the process.

Also provided herein in some embodiments is a method of predicting whether a therapeutically effective T cell therapy cannot be manufactured for a subject, comprising: (a) obtaining, for a subject having a disease or condition, a parameter of a marker or parameters of a combination of markers, wherein: (i) the parameter or parameters are obtained prior to when T cells for producing an autologous T cell therapy are collected from the subject, the T cell therapy comprising T cells comprising a recombinant receptor that binds to an antigen associated with the disease or condition; and (ii) the marker or the combination of markers is selected from any of one or more input composition markers, one or more process markers, one or more drug product markers, one or more subject immune profile markers, one or more subject fitness markers, one or more subject prior therapy markers, and one or more subject tumor burden markers of the subject; and (b) predicting if the T cell therapy will not be therapeutically effective based on one or more outputs of a process configured to predict, based on the marker or combination of markers, if the T cell therapy will not be therapeutically effective, wherein the predicting comprises providing the parameter or parameters as input to the process.

Also provided herein in some embodiments is a method of predicting whether a therapeutically effective T cell therapy can be manufactured for a subject, comprising: (a) obtaining, for a subject having a disease or condition, a parameter of a marker or parameters of a combination of markers, wherein: (i) the parameter or parameters are obtained prior to when T cells for producing an autologous T cell therapy are collected from the subject, the T cell therapy comprising T cells comprising a recombinant receptor that binds to an antigen associated with the disease or condition; and (ii) the marker or the combination of markers is selected from any of one or more subject immune profile markers, one or more subject fitness markers, one or more subject prior therapy markers, and one or more subject tumor burden markers of the subject; and (b) predicting if the T cell therapy will be therapeutically effective based on one or more outputs of a process configured to predict, based on the marker or combination of markers, if the T cell therapy will be therapeutically effective, wherein the predicting comprises providing the parameter or parameters as input to the process.

Also provided herein in some embodiments is a method of predicting whether a therapeutically effective T cell therapy cannot be manufactured for a subject, comprising: (a) obtaining, for a subject having a disease or condition, a parameter of a marker or parameters of a combination of markers, wherein: (i) the parameter or parameters are obtained prior to when T cells for producing an autologous T cell therapy are collected from the subject, the T cell therapy comprising T cells comprising a recombinant receptor that binds to an antigen associated with the disease or condition; and (ii) the marker or the combination of markers is selected from any of one or more subject immune profile markers, one or more subject fitness markers, one or more subject prior therapy markers, and one or more subject tumor burden markers of the subject; and (b) predicting if the T cell therapy will not be therapeutically effective based on one or more outputs of a process configured to predict, based on the marker or combination of markers, if the T cell therapy will not be therapeutically effective, wherein the predicting comprises providing the parameter or parameters as input to the process.

In some of any embodiments, the parameters of a combination of markers are obtained and provided as input to the process.

In some of any embodiments, the combination of markers comprises one or more input composition markers. In some embodiments, the one or more input composition markers are markers determined from cells of an input composition, wherein the input composition comprises peripheral blood mononuclear cells (PBMCs) selected from a biological sample from the subject, wherein T cells from the PBMCs are used for producing the T cell therapy. In some embodiments, the input composition markers are selected from (i) the percentage of CD3+ T cells in the biological sample; (ii) the ratio of CD4+ T cells to CD8+ T cells (CD4:CD8) in the biological sample; (iii) the percentage of CD57+ T cells in the biological sample; and (iv) the percentage of CD28+ T cells in the biological sample.

In some of any embodiments, the combination of markers comprises one or more process markers. In some embodiments, the one or more process markers are markers determined from cells of a composition during a process for manufacturing the T cell therapy. In some embodiments, the process for manufacturing the T cell therapy comprises activating T cells of the input composition, introducing the recombinant receptor into the activated T cells, and expanding the T cells to produce the T cell therapy. In some embodiments, the one or more process markers are selected from (i) the size of cells in the composition after activation and (ii) the number of total nucleated cells (TNC) at the end of the process.

In some of any embodiments, the combination of markers comprises one or more drug product markers. In some embodiments, the one or more drug product markers are markers determined from the cells of the T cell therapy. In some embodiments, the one or more drug product markers are selected from (i) the percentage of cells positive for the recombinant receptor (recombinant receptor+), (ii) the number of total cells positive for the recombinant receptor (total recombinant receptor); (iii) the vector copy number (VCN); and (iv) the level of potency of the T cell therapy. In some embodiments, the one or more drug product markers are selected from (i) the percentage of cells positive for the recombinant receptor (recombinant receptor+), (ii) the number of total cells positive for the recombinant receptor (total recombinant receptor); and (iii) the vector copy number (VCN).

In some of any embodiments, the combination of markers comprises one or more subject immune profile markers. In some of any embodiments, the one or more subject immune profile markers are selected from the (1) level in a blood sample of d-dimer, (2) level in a blood sample of fibrinogen, (3) level in a blood sample of lymphocytes, (4) level in a blood sample of monocytes, (5) ratio in a blood sample of monocytes to leukocytes, (6) level in a blood sample of red blood cells, and (7) level in a blood sample of white blood cells of the subject.

In some of any embodiments, the combination of markers comprises one or more subject fitness markers. In some of any embodiments, the one or more subject fitness markers are selected from the (8) age, (9) body mass index, (10) level in a blood sample of albumin, (11) level in a blood sample of alkaline phosphatase, (12) level in a blood sample of aspartate aminotransferase, (13) level in a blood sample of alanine aminotransferase, (14) level in a blood sample of direct bilirubin, (15) level in a blood sample of bilirubin, (16) level in a blood sample of creatinine, and (17) creatinine clearance of the subject.

In some of any embodiments, the combination of markers comprises one or more subject prior therapy markers. In some of any embodiments, the one or more subject prior therapy markers are selected from the (18) time since diagnosis, (19) number of prior therapies received, (20) time since prior autologous stem cell transplant, (21) time since prior corticosteroid therapy, (22) time since prior alkylating agent therapy, (23) time since prior topoisomerase inhibitor therapy, and (24) time since prior proteasome inhibitor therapy for the subject.

In some of any embodiments, the combination of markers comprises one or more subject tumor burden markers. In some of any embodiments, the one or more subject tumor burden markers are selected from the (25) percent in a blood sample of bone marrow plasma cells, (26) level in a blood sample of beta-2 microglobulin, (27) level in a blood sample of Immunoglobulin G, (28) level in a blood sample of lactate dehydrogenase, (29) ratio in a blood sample of kappa to lambda free light chain levels, (30) level in a blood sample of free light chain, (31) level in a blood sample of M-protein, (32) level in a blood sample of platelets, (33) level in a blood sample of sodium, and (34) level in a blood sample of soluble BCMA of the subject.

In some of any embodiments, the combination of markers comprises one or more subject immune profile markers and one or more subject tumor burden markers. In some of any embodiments, the combination of markers comprises one or more subject immune profile markers that are selected from markers (1)-(7) and one or more subject tumor burden markers that are selected from markers (25)-(34).

In some of any embodiments, the combination of markers comprises one or more subject immune profile markers, one or more subject prior therapy markers, and one or more subject tumor burden markers. In some of any embodiments, the combination of markers comprises one or more subject immune profile markers that are selected from markers (1)-(7), one or more subject prior therapy markers that are selected from markers (18)-(24), and one or more subject tumor burden markers that are selected from markers (25)-(34).

In some of any embodiments, the combination of markers comprises the (3) level in a blood sample of lymphocytes, (22) time since prior alkylating agent therapy, and (26) level in a blood sample of beta-2 microglobulin of the subject.

In some of any embodiments, the combination of markers comprises the (5) ratio in a blood sample of monocytes to leukocytes, (24) time since prior proteasome inhibitor therapy, (28) level in a blood sample of lactate dehydrogenase, and (31) level in a blood sample of M-protein of the subject.

In some of any embodiments, the process comprises a machine learning model trained to predict, based on the marker or combination of markers, if the subject is likely to exhibit the clinical response. In some of any embodiments, the process comprises a machine learning model trained to predict, based on the marker or combination of markers, if the subject is likely to not exhibit the clinical response.

In some of any embodiments, the process comprises a machine learning model trained to predict, based on the marker or combination of markers, e.g., parameters of the marker or combination of markers, if the T cell therapy will be therapeutically effective. In some of any embodiments, the process comprises a machine learning model trained to predict, based on the marker or combination of markers, e.g., parameters of the marker or combination of markers, if the T cell therapy is likely to not be therapeutically effective.

In some of any embodiments, the one or more outputs are outputs of, or are derived from outputs of, the machine learning model.

In some of any embodiments, the machine learning model is trained using parameters of the marker or parameters of the combination of markers from a plurality of subjects that were each administered a T cell therapy comprising T cells comprising a recombinant receptor that binds to an antigen associated with a disease or condition. In some embodiments, each of the plurality of subjects has the disease or condition.

In some of any embodiments, the machine learning model is trained using clinical responses of the plurality of subjects following administration of the T cell therapy.

In some of any embodiments, the disease or condition of the plurality of subjects is the same disease or condition of the subject. In some of any embodiments, the antigen associated with the disease or condition of the plurality of subjects is the same antigen associated with the disease or condition of the subject. In some of any embodiments, the recombinant receptor of the T cell therapy of the plurality of subjects is the same recombinant receptor of the T cell therapy of the subject. In some of any embodiments, the T cell therapy of the plurality of subjects is an autologous T cell therapy.

In some of any embodiments, the disease or condition is a cancer. In some of any embodiments, the disease or condition is a multiple myeloma. In some of any embodiments, the disease or condition is a relapsed/refractory multiple myeloma. In some of any embodiments, the antigen is a multiple myeloma-associated antigen. In some of any embodiments, the antigen is BCMA.

In some of any embodiments, prior to the obtaining of the parameter or parameters, the subject has received one or more prior therapies for treating the disease or condition. In some of any embodiments, the one or more prior therapies comprises one to three prior therapies. In some of any embodiments, the one or more prior therapies comprises at least three prior therapies. In some of any embodiments, the subject has relapsed or been refractory to the most recent of the one or more prior therapies.

In some of any embodiments, the one or more prior therapies comprises an immunomodulatory agent. In some of any embodiments, the immunomodulatory agent is selected from thalidomide, lenalidomide, and pomalidomide.

In some of any embodiments, the one or more prior therapies comprises a proteasome Inhibitor. In some of any embodiments, the proteasome inhibitor is selected from bortezomib, carfilzomib, and ixazomib.

In some of any embodiments, the one or more prior therapies comprises an anti-CD38 antibody. In some of any embodiments, the anti-CD38 antibody is or comprises daratumumab.

In some of any embodiments, the clinical response is progression free survival of greater than 2 months, 4 months, 6 months, or 8 months. In some of any embodiments, the clinical response is complete response (CR).

In some of any embodiments, the parameter or parameters are obtained within 6, 5, 4, 3, 2, or 1 month prior to when the T cell therapy is to be administered to the subject. In some of any embodiments, the parameter or parameters are obtained when or about when the subject is being screened for administration of the T cell therapy. In some of any embodiments, the parameter or parameters are obtained prior to when T cells for the T cell therapy are collected from the subject. In some of any embodiments, the obtaining comprises measuring the parameter or one of the more of the parameters from the subject.

In some of any embodiments, the recombinant receptor is a chimeric antigen receptor (CAR). In some of any embodiments, the CAR is an anti-BCMA CAR. In some of any embodiments, the CAR comprises an extracellular antigen-binding domain that binds to BCMA, a transmembrane domain, and an intracellular signaling region. In some of any embodiments, the intracellular signaling region comprises a cytoplasmic signaling domain of a CD3-zeta (CD3ζ) chain. In some of any embodiments, the intracellular signaling region comprises a costimulatory signaling domain. In some of any embodiments, the costimulatory signaling domain comprises an intracellular signaling domain of CD28, 4-1BB, or ICOS. In some of any embodiments, the costimulatory signaling domain is between the transmembrane domain and the cytoplasmic signaling domain of the CD3-zeta (CD3ζ) chain. In some of any embodiments, the transmembrane domain comprises a transmembrane domain from CD28 or CD8. In some of any embodiments, the transmembrane domain comprises a transmembrane domain from human CD28 or CD8. In some of any embodiments, the CAR further comprises an extracellular spacer between the antigen-binding domain and the transmembrane domain. In some of any embodiments, the spacer is from CD8. In some of any embodiments, the spacer is a CD8alpha hinge. In some of any embodiments, the transmembrane domain and the spacer are from CD8. In some of any embodiments, the CAR comprises the sequence set forth in SEQ ID NO:38.

In some of any embodiments, the T cell therapy is an autologous T cell therapy.

In some of any embodiments, the T cell therapy comprises idecabtagene vicleucel cells. In some of any embodiments, the T cell therapy is ABECMA®.

In some of any embodiments, the T cell therapy comprises ciltacabtagene autoleucel cells. In some of any embodiments, the T cell therapy is CARVYKTI™.

In some of any embodiments, the subject is a human.

In some of any embodiments, the subject is predicted as likely to not exhibit the clinical response, and the method further comprises selecting the subject for administration of an alternative treatment or treatment regimen.

In some of any embodiments, the T cell therapy is predicted as likely to not be therapeutically effective, and the method further comprises selecting the subject for administration of an alternative treatment or treatment regimen.

In some of any embodiments, the subject is predicted as likely to exhibit the clinical response, and the method further comprises selecting the subject for administration of the T cell therapy.

In some of any embodiments, the T cell therapy is predicted as likely to be therapeutically effective, and the method further comprises selecting the subject for administration of the T cell therapy.

In some of any embodiments, the method further comprises collecting T cells from the subject for producing the T cell therapy. In some of any embodiments, the T cells are collected after the subject is predicted as likely to exhibit the clinical response. In some of any embodiments, the T cells are collected after the T cell therapy is predicted as likely to be therapeutically effective. In some of any embodiments, the T cells are collected by apheresis. In some of any embodiments, the T cells are collected by leukapheresis.

In some embodiments, a T cell therapy predicted to be therapeutically effective has a percentage of CAR-expressing that is above an associated threshold level. In some embodiments, the associated threshold value is between or between about 30 percent and 55 percent. In some embodiments, the associated threshold value is between or between about 30 percent and 50 percent, 30 percent and 45 percent, 30 percent and 40 percent, or 30 percent and 35 percent. In some embodiments, the associated threshold value is between or between about 35 percent and 55 percent, 35 percent and 50 percent, 35 percent and 45 percent, or 35 percent and 40 percent. In some embodiments, the associated threshold value is between or between about 40 percent and 55 percent, 40 percent and 50 percent, or 40 percent and 45 percent. In some embodiments, the associated threshold value is between or between about 45 percent and 55 percent or 45 percent and 50 percent. In some embodiments, the associated threshold value is between or between about 50 percent and 55 percent. In some embodiments, the associated threshold value is between or between about 35 percent and 50 percent. In some embodiments, the associated threshold value is or is about 40 percent.

In some embodiments, a T cell therapy predicted to be therapeutically effective has a total number of CAR-expressing cells that is above an associated threshold level. In some embodiments, the associated threshold value is between or between about 0.5×10⁹ cells and 2.0×10⁹ cells. In some embodiments, the associated threshold value is between or between about 0.5×10⁹ cells and 1.5×10⁹ cells or 0.5×10⁹ cells and 1.0×10⁹ cells. In some embodiments, the associated threshold value is between or between about 1.0×10⁹ cells and 2.0×10⁹ cells or 1.0×10⁹ cells and 1.5×10⁹ cells. In some embodiments, the associated threshold value is between or between about 1.0×10⁹ cells and 1.75×10⁹ cells. In some embodiments, the associated threshold value is or is about 1.5×10⁹ cells.

In some embodiments, a T cell therapy predicted to be therapeutically effective has a vector copy number that is above an associated threshold value. In some embodiments, the associated threshold value is between or between about 4.5 copies/μg DNA and 8.5 copies/μg DNA. In some embodiments, the associated threshold value is between or between about 4.5 copies/μg DNA and 7.5 copies/μg DNA, 4.5 copies/μg DNA and 6.5 copies/μg DNA or 4.5 copies/μg DNA and 5.5 copies/μg DNA. In some embodiments, the associated threshold value is between or between about 5.5 copies/μg DNA and 8.5 copies/μg DNA, 5.5 copies/μg DNA and 7.5 copies/μg DNA or 5.5 copies/μg DNA and 6.5 copies/μg DNA. In some embodiments, the associated threshold value is between or between about 6.5 copies/μg DNA and 8.5 copies/μg DNA or 6.5 copies/μg DNA and 7.5 copies/μg DNA. In some embodiments, the associated threshold value is between or between about 7.5 copies/μg DNA and 8.5 copies/μg DNA. In certain embodiments, the associated threshold value is between or between about 4.5 copies/μg DNA and 8 copies/μg DNA. In certain embodiments, the associated threshold value is between or between about 5 copies/μg DNA and 6.5 copies/μg DNA. In some embodiments, the associated threshold value is or is about 6 copies/μg DNA.

Also provided herein in some embodiments is a method of treating a disease or condition in a human subject, comprising: (a) selecting a subject having a disease or condition for administration of a T cell therapy for treating the disease or condition, the T cell therapy comprising T cells comprising a recombinant receptor that binds to an antigen associated with the disease or condition, wherein the selecting is according to the method of any one of claims 116-120; and (b) administering the T cell therapy to the selected subject.

Also provided herein in some embodiments is a method of treating a disease or condition in a human subject, comprising administering a T cell therapy to a subject having a disease or condition, wherein: the T cell therapy comprises T cells comprising a recombinant receptor that binds to an antigen associated with the disease or condition; and the subject is selected according to the method of any one of claims 116-120 for administration of the T cell therapy.

Also provided herein in some embodiments is a method of treating a disease or condition in a human subject, comprising administering a T cell therapy to a human subject having a disease or condition, wherein: the T cell therapy comprises T cells comprising a recombinant receptor that binds to an antigen associated with the disease or condition; and the subject is a subject in which prior to administration of the T cell therapy to the subject, and for a marker or a combination of markers selected from (1) level in a blood sample of d-dimer, (2) level in a blood sample of fibrinogen, (3) level in a blood sample of lymphocytes, (4) level in a blood sample of monocytes, (5) ratio in a blood sample of monocytes to leukocytes, (6) level in a blood sample of red blood cells, (7) level in a blood sample of white blood cells, (8) age, (9) body mass index, (10) level in a blood sample of albumin, (11) level in a blood sample of alkaline phosphatase, (12) level in a blood sample of aspartate aminotransferase, (13) level in a blood sample of alanine aminotransferase, (14) level in a blood sample of direct bilirubin, (15) level in a blood sample of bilirubin, (16) level in a blood sample of creatinine, (17) creatinine clearance, (18) time since diagnosis, (19) number of prior therapies received, (20) time since prior autologous stem cell transplant, (21) time since prior corticosteroid therapy, (22) time since prior alkylating agent therapy, (23) time since prior topoisomerase inhibitor therapy, (24) time since prior proteasome inhibitor therapy, (25) percent in a blood sample of bone marrow plasma cells, (26) level in a blood sample of beta-2 microglobulin, (27) level in a blood sample of Immunoglobulin G, (28) level in a blood sample of lactate dehydrogenase, (29) ratio in a blood sample of kappa to lambda free light chain levels, (30) level in a blood sample of free light chain, (31) level in a blood sample of M-protein, (32) level in a blood sample of platelets, (33) level in a blood sample of sodium, and (34) level in a blood sample of soluble BCMA of the subject: (i) a parameter or one or more parameters of the subject for markers (3), (6)-(13), (16), (17), (20), (22)-(24), (28), (29), (32), and (33) are higher than an associated threshold level; or (ii) a parameter or one or more parameters of the subject for markers (1), (2), (4), (5), (14), (15), (18), (19), (21), (25)-(27), (30), (31), and (34) are lower than an associated threshold level.

In some of any embodiments, prior to administration of the T cell therapy to the subject, the subject has been determined to have: (i) a parameter or one or more parameters for markers (3), (6)-(13), (16), (17), (20), (22)-(24), (28), (29), (32), and (33) that are higher than an associated threshold level; or (ii) a parameter or one or more parameters for markers (1), (2), (4), (5), (14), (15), (18), (19), (21), (25)-(27), (30), (31), and (34) that are lower than an associated threshold level.

Also provided herein in some embodiments is a method of treating a disease or condition in a human subject, comprising administering a T cell therapy to a human subject having a disease or condition, wherein: the T cell therapy comprises T cells comprising a recombinant receptor that binds to an antigen associated with the disease or condition; and the subject is a subject in which prior to administration of the T cell therapy to the subject, and for a marker or a combination of markers selected from any of: one or more subject immune profile markers selected from (1) level in a blood sample of d-dimer, (2) level in a blood sample of fibrinogen, (3) level in a blood sample of lymphocytes, (4) level in a blood sample of monocytes, (5) ratio in a blood sample of monocytes to leukocytes, (6) level in a blood sample of red blood cells, and (7) level in a blood sample of white blood cells of the subject; one or more subject prior therapy markers selected from (18) time since diagnosis, (19) number of prior therapies received, (20) time since prior autologous stem cell transplant, (21) time since prior corticosteroid therapy, (22) time since prior alkylating agent therapy, (23) time since prior topoisomerase inhibitor therapy, and (24) time since prior proteasome inhibitor therapy for the subject; and one or more subject tumor burden markers selected from (25) percent in a blood sample of bone marrow plasma cells, (26) level in a blood sample of beta-2 microglobulin, (27) level in a blood sample of Immunoglobulin G, (28) level in a blood sample of lactate dehydrogenase, (29) ratio in a blood sample of kappa to lambda free light chain levels, (30) level in a blood sample of free light chain, (31) level in a blood sample of M-protein, (32) level in a blood sample of platelets, (33) level in a blood sample of sodium, and (34) level in a blood sample of soluble BCMA of the subject; (i) a parameter or one or more parameters of the subject for markers (3), (6), (7), (20), (22)-(24), (28), (29), (32), and (33) are higher than an associated threshold level; or (ii) a parameter or one or more parameters of the subject for markers (1), (2), (4), (5), (18), (19), (21), (25)-(27), (30), (31), and (34) are lower than an associated threshold level.

In some of any embodiments, prior to administration of the T cell therapy to the subject, the subject has been determined to have: (i) a parameter or one or more parameters for markers (3), (6), (7), (20), (22)-(24), (28), (29), (32), and (33) that are higher than an associated threshold level; or (ii) a parameter or one or more parameters for markers (1), (2), (4), (5), (18), (19), (21), (25)-(27), (30), (31), and (34) that are lower than an associated threshold level.

In some of any embodiments, the marker is or the combination of markers comprises one or more subject immune profile markers that are selected from markers (1)-(7).

In some of any embodiments, the marker is or the combination of markers comprises one or more subject fitness markers that are selected from markers (8)-(17).

In some of any embodiments, the marker is or the combination of markers comprises one or more subject prior therapy markers that are selected from markers (18)-(24).

In some of any embodiments, the marker is or the combination of markers comprises one or more subject tumor burden markers that are selected from markers (25)-(34).

In some of any embodiments, the combination of markers comprises one or more subject immune profile markers that are selected from markers (1)-(7) and one or more subject tumor burden markers that are selected from markers (25)-(34).

In some of any embodiments, the combination of markers comprises one or more subject immune profile markers that are selected from markers (1)-(7), one or more subject prior therapy markers that are selected from markers (18)-(24), and one or more subject tumor burden markers that are selected from markers (25)-(34).

In some of any embodiments, the combination of markers comprises the (3) level in a blood sample of lymphocytes, (22) time since prior alkylating agent therapy, and (26) level in a blood sample of beta-2 microglobulin of the subject.

In some of any embodiments, (i) the parameter of the subject for marker (3) is higher than an associated threshold level; (ii) the parameter of the subject for marker (22) is higher than an associated threshold level; or (iii) the parameter of the subject for marker (26) is lower than an associated threshold level. In some of any embodiments, (i) the parameter of the subject for marker (3) is higher than an associated threshold level; (ii) the parameter of the subject for marker (22) is higher than an associated threshold level; and (iii) the parameter of the subject for marker (26) is lower than an associated threshold level.

In some of any embodiments, the combination of markers comprise the (5) ratio in a blood sample of monocytes to leukocytes, (24) time since prior proteasome inhibitor therapy, (28) level in a blood sample of lactate dehydrogenase, and (31) level in a blood sample of M-protein of the subject.

In some of any embodiments, (i) the parameter of the subject for marker (5) is lower than an associated threshold level; (ii) the parameter of the subject for marker (24) is higher than an associated threshold level; (iii) the parameter of the subject for marker (28) is higher than an associated threshold level; or (iv) the parameter of the subject for marker (31) is lower than an associated threshold level. In some of any embodiments, (i) the parameter of the subject for marker (5) is lower than an associated threshold level; (ii) the parameter of the subject for marker (24) is higher than an associated threshold level; (iii) the parameter of the subject for marker (28) is higher than an associated threshold level; and (iv) the parameter of the subject for marker (31) is lower than an associated threshold level.

In some of any embodiments, the threshold level associated with marker (1) is between or between about 0.5 mg/L and 11 mg/L or between or between about 0.5 mg/L and 1.3 mg/L. In some of any embodiments, the threshold level associated with marker (2) is between or between about 2.2 g/L and 7.7 g/L or between or between about 4.2 g/L and 5.4 g/L. In some of any embodiments, the threshold level associated with marker (3) is between or between about 0.3×10⁹ cells/L and 1.0×10⁹ cells/L or between or between about 0.4×10⁹ cells/L and 0.7×10⁹ cells/L. In some of any embodiments, the threshold level associated with marker (4) is between or between about 0.2×10⁹ cells/L and 1.1×10⁹ cells/L or between or between about 0.4×10⁹ cells/L and 0.7×10⁹ cells/L. In some of any embodiments, the threshold level associated with marker (5) is between or between about 6.7 and 18 or between or between about 13 and 14. In some of any embodiments, the threshold level associated with marker (6) is between or between about 2.4×10¹² cells/L and 3.7×10¹² cells/L or between or between about 2.9×10¹² cells/L and 3.3×10¹² cells/L. In some of any embodiments, the threshold level associated with marker (7) is between or between about 2.1×10⁹ cells/L and 7.1×10⁹ cells/L or between or between about 2.9×10⁹ cells/L and 4.2×10⁹ cells/L. In some of any embodiments, the threshold level associated with marker (8) is between or between about 57 years and 66 years or between or between about 64 years and 66 years. In some of any embodiments, the threshold level associated with marker (9) is between or between about 22 kg/m² and 31 kg/m² or between or between about 23 kg/m² and 29 kg/m². In some of any embodiments, the threshold level associated with marker (10) is between or between about 31 g/L and 41 g/L or between or between about 36 g/L and 40 g/L. In some of any embodiments, the threshold level associated with marker (11) is between or between about 28 IU/L and 134 IU/L or between or between about 54 IU/L and 64 IU/L. In some of any embodiments, the threshold level associated with marker (12) is between or between about 7.3 IU/L and 49 IU/L or between or between about 16 IU/L and 26 IU/L. In some of any embodiments, the threshold level associated with marker (13) is between or between about 8 IU/L and 31 IU/L or between or between about 13 IU/L and 29 IU/L. In some of any embodiments, the threshold level associated with marker (14) is between or between about 1.4 μM and 2.7 μM or between or between about 1.8 μM and 2.2 μM. In some of any embodiments, the threshold level associated with marker (15) is between or between about 3.4 μM and 23 μM or between or between about 9.4 μM and 9.6 μM. In some of any embodiments, the threshold level associated with marker (16) is between or between about 46 μM and 114 μM or between or between about 52 μM and 80 μM. In some of any embodiments, the threshold level associated with marker (17) is between or between about 0.8 mL/s and 2.0 mL/s or between or between about 1.9 mL/s and 2.0 mL/s. In some of any embodiments, the threshold level associated with marker (18) is between or between about 2.2 years and 10 years or between or between about 5.5 years and 8.3 years. In some of any embodiments, the threshold level associated with marker (19) is between or between about 4 and 11 or between or between about 4 and 5. In some of any embodiments, the threshold level associated with marker (20) is between or between about 26 days and 3205 days or between or between about 641 days and 2941 days. In some of any embodiments, the threshold level associated with marker (21) is between or between about 12 days and 2257 days or between or between about 42 days and 59 days. In some of any embodiments, the threshold level associated with marker (22) is between or between about 11 days and 493 days or between or between about 230 days and 244 days. In some of any embodiments, the threshold level associated with marker (23) is between or between about 87 days and 3356 days or between or between about 474 days and 676 days. In some of any embodiments, the threshold level associated with marker (24) is between or between about 11 days and 658 days or between or between about 51 days and 170 days. In some of any embodiments, the threshold level associated with marker (25) is between or between about 21% and 100% or between or between about 56% and 80%. In some of any embodiments, the threshold level associated with marker (26) is between or between about 2.7 mg/L and 7.7 mg/L or between or between about 3.2 mg/L and 4.6 mg/L. In some of any embodiments, the threshold level associated with marker (27) is between or between about 2.8 g/L and 75 g/L or between or between about 14 g/L and 35 g/L. In some of any embodiments, the threshold level associated with marker (28) is between or between about 150 IU/L and 319 IU/L or between or between about 181 IU/L and 319 IU/L. In some of any embodiments, the threshold level associated with marker (29) is between or between about 0.003 and 763 or between or between about 8.7 and 211. In some of any embodiments, the threshold level associated with marker (30) is between or between about 0.008 g/L and 12 g/L or between or between about 0.2 g/L and 1.0 g/L. In some of any embodiments, the threshold level associated with marker (31) is between or between about 4.3 g/L and 32 g/L or between or between about 5.3 g/L and 12 g/L. In some of any embodiments, the threshold level associated with marker (32) is between or between about 53×10⁹ cells/L and 212×10⁹ cells/L or between or between about 156×10⁹ cells/L and 181×10⁹ cells/L. In some of any embodiments, the threshold level associated with marker (33) is between or between about 132 mM and 141 mM or between or between about 136 mM and 138 mM. In some of any embodiments, the threshold level associated with marker (34) is between or between about 35 ng/mL and 1300 ng/mL or between or between about 170 ng/mL and 654 ng/mL.

In some of any embodiments, the T cell therapy comprises between at or about 5×10⁷ recombinant receptor-comprising T cells and at or about 1×10⁹ recombinant receptor-comprising T cells or between at or about 1×10⁸ recombinant receptor-comprising T cells and at or about 1×10⁹ recombinant receptor-comprising T cells. In some of any embodiments, the T cell therapy comprises at or about 4.5×10⁸ recombinant receptor-comprising T cells.

In some of any embodiments, the T cell therapy is administered by an intravenous infusion.

In some of any embodiments, the T cell therapy is an autologous T cell therapy.

In some of any embodiments, the subject is subject to apheresis to collect T cells for the T cell therapy, and wherein the selection occurs prior to the apheresis. In some of any embodiments, the subject is subject to apheresis to collect T cells for the T cell therapy, and wherein the determination occurs prior to the apheresis.

In some of any embodiments, the selection is within 6, 5, 4, 3, 2, or 1 month prior to when the T cell therapy is administered to the subject. In some of any embodiments, the determination is within 6, 5, 4, 3, 2, or 1 month prior to when the T cell therapy is administered to the subject.

In some of any embodiments, the selection occurs at screening of the subject for administration of the T cell therapy. In some of any embodiments, the determination occurs at screening of the subject for administration of the T cell therapy.

In some of any embodiments, the disease or condition is a hematologic disease. In some of any embodiments, the disease or condition is a cancer. In some of any embodiments, the disease or condition is a multiple myeloma. In some of any embodiments, the disease or condition is a relapsed/refractory multiple myeloma.

In some of any embodiments, the antigen associated with the disease or condition is a multiple myeloma-associated antigen. In some of any embodiments, the antigen associated with the disease or condition is human BCMA.

In some of any embodiments, the recombinant receptor is a chimeric antigen receptor (CAR). In some of any embodiments, the CAR is an anti-BCMA CAR. In some of any embodiments, the CAR comprises an extracellular antigen-binding domain that binds to BCMA, a transmembrane domain, and an intracellular signaling region. In some of any embodiments, the intracellular signaling region comprises a cytoplasmic signaling domain of a CD3-zeta (CD3ζ) chain. In some of any embodiments, the intracellular signaling region comprises a costimulatory signaling domain. In some of any embodiments, the costimulatory signaling domain comprises an intracellular signaling domain of CD28, 4-1BB, or ICOS. In some of any embodiments, the costimulatory signaling domain is between the transmembrane domain and the cytoplasmic signaling domain of the CD3-zeta (CD3ζ) chain. In some of any embodiments, the transmembrane domain comprises a transmembrane domain from CD28 or CD8. In some of any embodiments, the transmembrane domain comprises a transmembrane domain from human CD28 or CD8. In some of any embodiments, the CAR further comprises an extracellular spacer between the antigen-binding domain and the transmembrane domain. In some of any embodiments, the spacer is from CD8. In some of any embodiments, the spacer is a CD8alpha hinge. In some of any embodiments, the transmembrane domain and the spacer are from CD8. In some of any embodiments, the CAR comprises the sequence set forth in SEQ ID NO:38.

In some of any embodiments, the T cell therapy is a CAR T cell therapy.

In some of any embodiments, the T cell therapy comprises idecabtagene vicleucel cells. In some of any embodiments, the T cell therapy is ABECMA®.

In some of any embodiments, the T cell therapy comprises ciltacabtagene autoleucel cells. In some of any embodiments, the T cell therapy is CARVYKTI™.

In some of any embodiments, the subject is a human.

In some of any embodiments, the method further comprises administering a bridging therapy to the subject, wherein the bridging therapy is administered to the subject between the selection and the administration of the T cell therapy. In some of any embodiments, the method further comprises administering a bridging therapy to the subject, wherein the bridging therapy is administered to the subject between the determination and the administration of the T cell therapy.

In some of any embodiments, prior to the administration of the T cell therapy, the subject has received one or more prior therapies for treating the disease or condition. In some of any embodiments, the one or more prior therapies comprises one to three prior therapies. In some of any embodiments, the one or more prior therapies comprises at least three prior therapies. In some of any embodiments, the subject has relapsed or been refractory to the most recent of the one or more prior therapies.

In some of any embodiments, the one or more prior therapies comprises an immunomodulatory agent. In some of any embodiments, the immunomodulatory agent is selected from thalidomide, lenalidomide, and pomalidomide.

In some of any embodiments, the one or more prior therapies comprises a proteasome inhibitor. In some of any embodiments, the proteasome inhibitor is selected from bortezomib, carfilzomib, and ixazomib.

In some of any embodiments, the one or more prior therapies comprises an anti-CD38 antibody. In some of any embodiments, the anti-CD38 antibody is or comprises daratumumab.

In some of any embodiments, the blood sample is a whole blood sample. In some embodiments, the blood sample is a plasma sample. In some embodiments, the blood sample is a serum sample.

### BRIEF DESCRIPTION OF DRAWINGS

FIG. 1 shows unsupervised clustering of Uniform Manifold Approximation and Projection (UMAP) components. Ten key manufacturing variables were dimensionally reduced, unsupervised clustering was applied to the first two UMAP components. Clusters were then tested for associations with downstream clinical outcomes to connect clusters to clinical profiles.

FIG. 2A details the ten key manufacturing variables, categorized as peripheral blood mononuclear cell (PBMC), in-process parameters, and drug product (DP) variables. FIG. 2B shows the distinct cell therapy manufacturing trajectories via unsupervised clustering (cluster 1, n=17; cluster 2, n=94, cluster 3, n=24; cluster 4, n=29).

FIG. 3A demonstrates Kaplan-Meier curves of median progression-free survival (mPFS) by manufacturing clusters. FIG. 3B shows the proportion of patients with best overall response of complete response (CR) or better in clusters. Efficacy is based on investigator response assessments.

FIG. 4A shows the percentage of CD3+ cells in PBMCs isolated from leukapheresis material obtained from relapsed and refractory multiple myeloma (RRMM). FIG. 4B shows the CD4 to CD8 ratio in the same PBMCs. FIGS. 4C-4D demonstrate the percentage of CD57+ cells and CD28+ cells, respectively, in the same PBMCs.

FIG. 5A shows the day 5 cell size. FIG. 5B shows the total number of nucleate cells (TNC).

FIG. 6A shows the percentage of CAR cells in the resulting drug product. FIG. 6B shows the total number of CAR cells (CAR yield). FIG. 6C shows the vector copy number (VCN). FIG. 6D shows the CAR T cell potency.

FIG. 7A shows the serum level of albumin in the different manufacturing clusters. FIG. 7B shows the serum absolute lymphocyte count (ALC) in the different manufacturing clusters. FIG. 7C shows the serum platelet count in the different manufacturing clusters. FIG. 7D shows the serum level of beta-2 microglobulin (B2M) in the different manufacturing clusters. FIG. 7E shows the serum level of mononuclear protein (M-protein) in the different manufacturing clusters. FIG. 7F shows the proportion of patients whose last exposure to prior alkylator therapy is less than 6 months before apheresis in the different manufacturing clusters.

FIG. 8 demonstrates the tumor burden changes between screening and baseline, assessed by serum B-cell maturation agent (sBCMA) levels at screening or at infusion in the different manufacturing clusters.

FIGS. 9A and 9B demonstrates the median progression-free survival for clusters 1 and 2, further subdivided by the highest or lowest quantile sBCMA at screening or at infusion respectively.

FIGS. 10A and 10B demonstrates the correlation between absolute lymphocyte count at screening and time-since-last-exposure to alkylating agent therapy or proteasome inhibitor therapy respectively.

FIG. 11 shows the supervised machine learning model (using random regression and survival forests) used to list the top variables important for the two endpoints of interest.

FIG. 12 shows the univariate receiver operating characteristic area under the curve values (ROC AUCs) for predicting patient association with manufacturing cluster 1 or with manufacturing cluster 1 or 2.

FIG. 13 shows the multivariate receiver operating character area under the curves (ROC AUCs) for predicting patient association with manufacturing cluster 1 or with manufacturing cluster 1 or 2.

FIGS. 14A-14C show the predictors for the model predicting patient association with manufacturing cluster 1. FIG. 14A shows serum absolute lymphocyte count (ALC). FIG. 14B shows serum beta-2-microglobulin (B2M) level. FIG. 14C shows the percentage of patients who last exposure to prior alkylator therapy was less than 4 months before apheresis.

FIGS. 15A-15D show the predictors for the model predicting patient association with manufacturing cluster 1 or 2. FIG. 15A is the monocyte to leukocyte ratio (mono:leuk). FIG. 15B shows the serum mononuclear protein (M-protein) level. FIG. 15C shows the serum lactate dehydrogenase (LDH) level. FIG. 15D shows the percentage of patients whose last exposure to prior proteasome inhibitor therapy was less than 4 months before apheresis.

### DETAILED DESCRIPTION

Provided herein are methods for predicting if a subject will or will not exhibit a clinical response to administration of a T cell therapy (e.g., therapeutic cell composition). Also provided herein are methods for predicting if a therapeutically effective T cell therapy can or cannot be produced for the subject. In some embodiments, the predicting is prior to the subject being treated. In some embodiments, the predicting is prior to T cells for the T cell therapy being collected from the subject. In some embodiments, the provided methods relate to determining effective selection of patients for T cell therapy, manufacturing of a T cell therapy, and administration of a T cell therapy, e.g., a therapeutic cell composition.

In some aspects, predicting before treatment if a subject will exhibit a clinical response to administration of a T cell therapy affords several advantages. For instance, if a subject is predicted to not exhibit a clinical response, an alternative treatment regimen for the subject, with or without administration of the T cell therapy, can be determined. For instance, an alternative treatment could include a combination therapy that includes the T cell therapy in combination with a separate therapeutic agent, or the alternative treatment could not include the T cell therapy. In some instances, a subject predicted to not exhibit a clinical response to administration of a T cell therapy can be immediately placed on an alternative treatment regimen, rather than, for instance, delaying treatment for manufacturing of a T cell therapy that may not be effective in treating the patient. In some instances, additional procedures such as apheresis for collecting T cells for the T cell therapy can be avoided in subjects predicted to not exhibit a clinical response. Similar considerations apply to, and similar advantages are also afforded by, the provided methods involving predicting if a therapeutic T cell therapy can be manufactured for a subject.

In some embodiments, the predictions made by the provided methods are based on subject markers, for instance characteristics of a subject that are related to their immune profile or health, fitness, prior therapies they have received for treatment of their disease or condition, such as cancer, or level of tumor burden. In some aspects, values, e.g., parameters, of the subject markers that are used for prediction can be readily identified or collected using medical history or standard laboratory tests. In some aspects, values of the subject markers can be identified or collected by physicians when, for example, subjects are being screened for manufacture and administration of the T cell therapy. In some embodiments, parameters of the subject markers can be identified or collected using equipment readily available in any clinical setting and without specialized equipment. In some embodiments, parameters of the subject markers can be identified or collected without needing to perform intensive or invasive procedures. For instance, in some embodiments, parameters of the subject markers can be obtained using a blood sample collected from the subject, for instance a whole blood, serum, or plasma sample collected by a physician.

In some embodiments, the subject markers used for prediction by the provided methods include subject markers pertaining to subjects' immune profile and to their level of tumor burden. In some embodiments, the subject markers used for prediction include those pertaining to subjects' immune profile, to their level of tumor burden, and to the prior therapies received for treatment of the disease or condition. In some aspects, these combinations of subject markers are based on the results described herein demonstrating that subject markers spanning these categories can be used to accurately predict whether or not a subject will exhibit a clinical response, or whether or not a therapeutically effective T cell therapy can be manufactured for the subject. In some aspects, these combinations are based on the results described herein indicating that these categories may provide orthogonal information regarding the subject. In some aspects, these combinations can provide an overall profile of the subject while requiring only a limited number of markers per category, for instance one marker per category.

In some aspects, evaluation of the subject markers used for prediction can be used to determine an alternative treatment plan for subjects predicted not to exhibit a clinical response. As an example, if it is determined that a subject is predicted not to exhibit a clinical response in part due to a too-short period of time between screening and a prior therapy, such as a prior alkylating agent, topoisomerase inhibitor, or proteasome inhibitor therapy, apheresis for collection of T cells for the T cell therapy can be delayed for the subject until a longer period of time has elapsed since receiving the prior therapy. In another example, if it is determined that a subject is predicted not to exhibit a clinical response in part due to reduced lymphocyte counts at screening, apheresis for collection of T cells for the T cell therapy can be delayed for the subject until lymphocyte counts are increased for the subject. In some embodiments, the subject can be administered an additional therapy for increasing lymphocyte counts until the subject is predicted to exhibit a clinical response. In another example, if it is determined that a subject is predicted not to exhibit a clinical response in part due to high tumor burden at screening, apheresis for collection of T cells for the T cell therapy can be delayed for the subject until tumor burden is reduced for the subject. In some embodiments, the subject can be administered an additional therapy for reducing tumor burden until the subject is predicted to exhibit a clinical response. In some embodiments, additional therapies (e.g., those for influencing the immune profile or reducing the tumor burden of the subject) can also be administered to subjects predicted to exhibit a clinical response. For instance, in some embodiments, a bridging therapy for reducing or maintaining levels of tumor burden can be administered to a subject predicted to exhibit a clinical response following apheresis and during the period of time prior to administration of the T cell therapy.

In some embodiments, the methods provided herein include using thresholds of one or a combination of markers to determine (e.g., predict) a subject's clinical response to a T cell therapy, e.g., therapeutic composition, such as a complete response (CR), a partial response (PR), a durable response (e.g., durability of response, DOR), a best overall response (BOR), a toxicity response, a progression-free-survival (PFS) or median progression-free survival (mPFS), and/or a pharmacokinetic response, based on markers associated with the cells, e.g., peripheral blood mononuclear cell (PBMCs), of an input composition used for producing the T cell therapy, e.g., input composition markers, markers associated with the cells of the manufactured T cell therapy, e.g., drug product (DP) markers, markers associated with cells during manufacturing of the T cell therapy, e.g., process markers, and markers associated with the subject, e.g., prior therapy, tumor burden, immune profile, or fitness markers.

In some embodiments, the methods provided herein include machine learning models trained to determine (e.g., predict) a subject's clinical response to a cell therapy, e.g., therapeutic composition, such as a complete response (CR), a partial response (PR), a durable response (e.g., durability of response, DOR), a best overall response (BOR), a toxicity response, a progression-free-survival (PFS) or median progression-free survival (mPFS), and/or a pharmacokinetic response, based on markers associated with the cells, e.g., peripheral blood mononuclear cell (PBMCs), of an input composition used for producing the T cell therapy, e.g., input composition markers, markers associated with the cells of the manufactured T cell therapy, e.g., drug product (DP) markers, markers associated with cells during manufacturing of the T cell therapy, e.g., process markers, and markers associated with the subject, e.g., prior therapy, tumor burden, immune profile, or fitness markers.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Methods for Predicting Clinical Response or Manufacturing Outcomes

In some embodiments, the provided methods allow for predicting clinical responses or manufacturing outcomes in subjects to be treated with a T cell therapy prior to treatment with the T cell therapy based on markers, for instance markers associated with the cells, e.g., peripheral blood mononuclear cell (PBMCs), of an input composition used for producing the T cell therapy, e.g., input composition markers, markers associated with the cells of the manufactured T cell therapy, e.g., drug product (DP) markers, markers associated with cells during manufacturing of the T cell therapy, e.g., process markers, and markers associated with the subject, e.g., prior therapy, tumor burden, immune profile, or fitness markers. Having this type of information at an early stage, e.g., prior to treatment, allows for the development of treatment strategies (e.g., combination treatment, dosing) prior to treating the subject, thereby increasing the probability of a subject having a positive or advantageous clinical response (e.g., durable response, progression free survival).

In some embodiments, understanding the relationship (e.g., association) between markers, e.g., subject markers, with clinical responses in a subject, as well as an ability to determine or predict, prior to treatment, clinical responses in a subject to treatment with a T cell therapy can inform treatment strategy. For example, treatment regimens, e.g., predetermined treatment regimens, may be altered or maintained depending on the anticipated clinical response. In some embodiments, maintaining the predetermined treatment regimen or altering the treatment regimen may be useful in generating positive clinical responses, e.g., complete response (CR), partial response (PR), duration of response (DOR) or progression free survival (PFS) of at least a certain length, or no toxicity. In some embodiments, if a subject is predicted as likely to not exhibit a clinical response, the subject is selected for administration of an alternative treatment or treatment regimen.

In some embodiments, if the subject to be treated is predicted as likely to not exhibit a clinical response, a treatment strategy that includes an additional treatment may be considered. In some embodiments, the T cell therapy is administered as part of a combination treatment, such as simultaneously with or sequentially with, in any order, another therapeutic intervention, such as an antibody or engineered cell or receptor or agent, such as a cytotoxic or therapeutic agent. The T cell therapy in some embodiments are co-administered with one or more additional therapeutic agents or in connection with another therapeutic intervention, either simultaneously or sequentially in any order. In some contexts, the T cell therapy is co-administered with another therapy sufficiently close in time such that the T cell therapy enhances the effect of one or more additional therapeutic agents, or vice versa. In some embodiments, the T cell therapy is administered prior to the one or more additional therapeutic agents. In some embodiments, the T cell therapy is administered after the one or more additional therapeutic agents. In some embodiments, the one or more additional agents include a cytokine, such as IL-2, for example, to enhance persistence. In some embodiments, the methods include administration of a chemotherapeutic agent.

In some embodiments, the methods include administration of a chemotherapeutic agent, e.g., a conditioning chemotherapeutic agent, for example, to reduce tumor burden prior to the administration. In some embodiments, the methods include administering a bridging therapy, such as a chemotherapeutic agent, to the subject. In some embodiments, the bridging therapy is administered to the subject before the administration of the T cell therapy. In some embodiments, the bridging therapy is administered to the subject after the subject is selected for administration of the T cell therapy.

In some embodiments, the combination therapy includes administration of a kinase inhibitor, such as a BTK inhibitor (e.g., ibrutinib or acalibrutinib); an inhibitor or a tryptophan metabolism and/or kynurenine pathway, such as an inhibitor of indoleamine 2,3-dioxygenase-1 (IDO1) (e.g., epacadostat); an immunomodulatory agent, such as an immunomodulatory imide drug (IMiD), including a thalidomide or thalidomide derivative (e.g., lenalidomide or pomalidomide); or a check point inhibitor, such as an anti-PD-L1 antibody (e.g., durvalumab). In some embodiments, any of the foregoing agents are administered to the subject as a bridging therapy.

Exemplary combination therapies and methods are described in published international applications WO 2018/085731, WO 2018/102785, WO 2019/213184, WO 2018/071873, WO 2018/102786, WO 2018/204427, WO 2019/152743, which are incorporated by reference in their entirety.

### A. Markers

It is contemplated that clinical responses of a subject to treatment with a T cell therapy, as well as whether a therapeutically effective T cell therapy can be produced for the subject, depend upon many factors, for instance markers associated with the cells, e.g., peripheral blood mononuclear cell (PBMCs), of an input composition used for producing the T cell therapy, e.g., input composition markers, markers associated with the cells of the manufactured T cell therapy, e.g., drug product (DP) markers, markers associated with cells during manufacturing of the T cell therapy, e.g., process markers, and markers associated with the subject, e.g., prior therapy, tumor burden, immune profile, or fitness markers. In some aspects, the provided methods use one or a combination of the foregoing markers for predicting if a subject will or will not exhibit a clinical response to administration of a T cell therapy. In some aspects, the provided methods use one or a combination of the foregoing markers for predicting if a therapeutically effective T cell therapy can or cannot be produced for the subject. In some embodiments, the markers are compared to predetermined thresholds or are used for prediction by a machine learning process.

In some embodiments, a marker is a measurable characteristic that can be used to predict if a subject is likely to or likely to not exhibit a clinical response. In some embodiments, a marker is a measurable characteristic that can be used to predict if a therapeutically effective T cell therapy can be produced for the subject. In some embodiments, the marker is a measurable characteristic of a subject, input composition, drug product, or cells during manufacturing. In some embodiments, a combination of markers are all measurable characteristics of a single source, for instance in the case of a combination of all subject markers. In some embodiments, a combination of markers includes measurable characteristics from multiple sources, such as some from a subject, and some from an input composition.

In some embodiments, a parameter is a value that is measured for a marker. For instance, in the case of a subject marker, the parameter for the marker can be obtained or measured from the subject. For example, a subject could have, for the marker “age,” an associated parameter that is a numeric value in years.

In some embodiments, the markers can include cell phenotypes. In some embodiments, cell phenotype is determined by assessing the presence or absence of one or more specific molecules, including surface molecules and/or molecules that may accumulate or be produced by the cells or a subpopulation of cells within the input composition or within the T cell therapy. In some embodiments, cell phenotype may include cell activity, such as production of a factor (e.g., cytokine) in response to a stimulus. In some embodiments, the production of a factor (e.g., cytokine) is in response to recombinant receptor-dependent activation. In some embodiments, recombinant receptor-dependent activity of cells of the T cell therapy is determined by assessing one or more specific molecules (e.g., cytokines) that may accumulate or be produced by the cells or a subpopulation of cells within the T cell therapy. In some embodiments, recombinant receptor-dependent activity is assessed by determining the cytolytic activity of the cells of the T cell therapy.

In some embodiments, markers of the input composition and/or the T cell therapy include a determination, detection, quantification, or other assessment of a phenotype of the cells (e.g., surface molecule, cytokine, recombinant receptor). In particular embodiments, markers of the composition (e.g., input composition, T cell therapy) include a determination, detection, quantification, or other assessment of the presence, absence, degree of expression or level of a specific molecule (e.g., surface molecule, cytokine, recombinant receptor). In some embodiments, the percentage, number, ratio, and/or proportion of cells having an attribute is determined. In some embodiments, the percentage, number, ratio, and/or proportion of cells having an attribute is a T cell therapy marker, e.g., drug product marker, or an input composition marker which can be used as input for a machine learning algorithm provided herein.

In some embodiments, the marker is a phenotype, e.g., cell phenotype. In some embodiments, the T cell therapy marker or the input composition marker is a phenotype indicative of viability of a cell. In some embodiments, the phenotype is indicative of absence of apoptosis, absence of early stages of apoptosis or absence of late stages of apoptosis. In some embodiments, the phenotype is the absence of a factor indicative of absence of apoptosis, early apoptosis or late stages of apoptosis. In some embodiments, the phenotype is a phenotype of a sub-population or subset of T cells, such as recombinant receptor-expressing T cells (e.g., CAR⁺ T cells), CD8⁺ T cells, or CD4⁺ T cells in the T cell therapy. In some embodiments, the phenotype is indicative of cell activation. In some embodiments, the phenotype is a phenotype of cells that are not activated and/or that lack or are reduced for or low for expression of one or more activation marker. In some embodiments, the phenotype is a phenotype of cells that are not exhausted and/or that lack or are reduced for or low for expression of one or more exhaustion markers.

In some embodiments, the phenotype is the production of one or more cytokines. In some embodiments, for example when the cytokine is produced and/or secreted by an engineered cell of the T cell therapy in response to engagement of a recombinant receptor expressed by the cell with its antigen, this activity is referred to as recombinant receptor-dependent activity. In some embodiments, the T cell therapy marker, e.g., drug product marker, is recombinant receptor-dependent activity.

In some embodiments, the production of one or more cytokines is measured, detected, and/or quantified by intracellular cytokine staining. In particular embodiments, the phenotype is the lack of the production of the cytokine. In particular embodiments, the phenotype is positive for or is a high level of production of a cytokine. Intracellular cytokine staining (ICS) by flow cytometry is a technique well-suited for studying cytokine production at the single-cell level. It detects the production and accumulation of cytokines within the endoplasmic reticulum after cell stimulation, allowing for the identification of cell populations that are positive or negative for production of a particular cytokine or for the separation of high producing and low producing cells based on a threshold. ICS can also be used in combination with other flow cytometry protocols for immunophenotyping using cell surface markers or with MHC multimers to access cytokine production in a particular subgroup of cells, making it an extremely flexible and versatile method. Other single-cell techniques for measuring or detecting cytokine production include, but are not limited to, ELISPOT, limiting dilution, and T cell cloning.

In particular embodiments, the markers include recombinant receptor-dependent activity. In some embodiments, the activity is a recombinant receptor, e.g., a CAR, dependent activity that is or includes the production and/or secretion of a soluble factor. In certain embodiments, the soluble factor is a cytokine or a chemokine.

Suitable techniques for the measurement of the production or secretion of a soluble factor are known in the art. Production and/or secretion of a soluble factor can be measured by determining the concentration or amount of the extracellular amount of the factor, or determining the amount of transcriptional activity of the gene that encodes the factor. Suitable techniques include, but are not limited to assays such as an immunoassay, an aptamer-based assay, a histological or cytological assay, an mRNA expression level assay, an enzyme linked immunosorbent assay (ELISA), alphalisa assay, immunoblotting, immunoprecipitation, radioimmunoassay (RIA), immunostaining, flow cytometry assay, surface plasmon resonance (SPR), chemiluminescence assay, lateral flow immunoassay, inhibition assay or avidity assay, protein microarrays, high-performance liquid chromatography (HPLC), Meso Scale Discovery (MSD) electrochemiluminescence and bead based multiplex immunoassays (MIA). In some embodiments, the suitable technique may employ a detectable binding reagent that specifically binds the soluble factor.

In some embodiments, the phenotype is indicated by the presence, absence, or level of expression in a cell of one or more specific molecules, such as certain surface markers indicative of the phenotype, e.g., surface proteins, intracellular markers indicative of the phenotype, or nucleic acids indicative of the phenotype or other molecules or factors indicative of the phenotype. In some embodiments, the phenotype is or comprises a positive or negative expression of the one or more of specific molecules. In some embodiments, the specific molecules include, but are not limited to, a surface marker, e.g., a membrane glycoprotein or a receptor; a marker associated with apoptosis or viability; or a specific molecule that indicates the status of an immune cells, e.g., a marker associated with activation, exhaustion, or a mature or naive phenotype. In some embodiments, any known method for assessing or measuring, counting, and/or quantifying cells based on specific molecules can be used to determine the number of cells of the phenotype in the composition (e.g., input composition, T cell therapy).

In some embodiments, a phenotype is or includes a positive or negative expression of one or more specific molecules in a cell. In some embodiments, the positive expression is indicated by a detectable amount of the specific molecule in the cell. In certain embodiments, the detectable amount is any detected amount of the specific molecule in the cell. In particular embodiments, the detectable amount is an amount greater than a background, e.g., background staining, signal, etc., in the cell. In certain embodiments, the positive expression is an amount of the specific molecule that is greater than a threshold, e.g., a predetermined threshold. Likewise, in particular embodiments, a cell with negative expression of a specific molecule may be any cell not determined to have positive expression, or is a cell that lacks a detectable amount of the specific molecule or a detectable amount of the specific molecule above background. In some embodiments, the cell has negative expression of a specific molecule if the amount of the specific molecule is below a threshold. One of skill in the art will understand how to define a threshold to define positive and/or negative expression for a specific molecule as a matter of routine skill, and that the thresholds may be defined according to specific parameters of, for example, but not limited to, the assay or method of detection, the identity of the specific molecule, reagents used for detection, and instrumentation.

Examples of methods that can be used to detect a specific molecule and/or analyze a phenotype of the cells include, but are not limited to, biochemical analysis; immunochemical analysis; image analysis; cytomorphological analysis; molecule analysis such as PCR, sequencing, high-throughput sequencing, determination of DNA methylation; proteomics analysis such as determination of protein glycosylation and/or phosphorylation pattern; genomics analysis; epigenomics analysis (e.g., ChIP-seq or ATAC-seq); transcriptomics analysis (e.g., RNA-seq); and any combination thereof. In some embodiments, the methods can include assessment of immune receptor repertoire, e.g., repertoire of T cell receptors (TCRs). In some aspects, determination of any of the phenotypes can be assessed in high-throughput, automated and/or by single-cell-based methods. In some aspects, large-scale or genome-wide methods, can be used to identify one or more molecular signatures. In some aspects, one or more molecular signatures, e.g., expression of specific RNA or proteins in the cell, can be determined. In some embodiments, molecular features of the phenotype analyzed by image analysis, PCR (including the standard and all variants of PCR), microarray (including, but not limited to DNA microarray, MMchips for microRNA, protein microarray, cellular microarray, antibody microarray, and carbohydrate array), sequencing, biomarker detection, or methods for determining DNA methylation or protein glycosylation pattern. In particular embodiments, the specific molecule is a polypeptide, i.e. a protein. In some embodiments, the specific molecule is a polynucleotide.

In some embodiments, positive or negative expression of a specific molecule is determined by incubating cells with one or more antibodies or other binding agents that specifically bind to one or more surface markers expressed or expressed (marker⁺) at a relatively higher level (marker^{high}) on the positively or negatively selected cells, respectively. In particular embodiments, the positive or negative expression is determined by flow cytometry, immunohistochemistry, or any other suitable method for detecting specific markers.

In particular embodiments, expression of a specific molecule is assessed with flow cytometry. Flow cytometry is a laser- or impedance-based, biophysical technology employed in cell counting, cell sorting, biomarker detection and protein engineering, by suspending cells in a stream of fluid and passing them by an electronic detection apparatus. It allows simultaneous multiparametric analysis of the physical and chemical characteristics of up to thousands of particles per second.

The data generated by flow-cytometers can be plotted in a single dimension, to produce a histogram, or in two-dimensional dot plots or even in three dimensions. The regions on these plots can be sequentially separated, based on fluorescence intensity, by creating a series of subset extractions, termed “gates.” Specific gating protocols exist for diagnostic and clinical purposes especially in relation to immunology. Plots are often made on logarithmic scales. Because different fluorescent dyes' emission spectra overlap, signals at the detectors have to be compensated electronically as well as computationally. Data accumulated using the flow cytometer can be analyzed using software, e.g., JMP (statistical software), WinMDI, Flowing Software, and web-based Cytobank), Cellcion, FCS Express, FlowJo, FACSDiva, CytoPaint (aka Paint-A-Gate), VenturiOne, CellQuest Pro, Infinicyt or Cytospec.

Flow Cytometry is a standard technique in the art and one of skill would readily understand how to design or tailor protocols to detect one or more specific molecules and analyze the data to determine the expression of one or more specific molecules in a population of cells. Standard protocols and techniques for flow cytometry are found in Loyd “Flow Cytometry in Microbiology; Practical Flow Cytometry by Howard M. Shapiro; Flow Cytometry for Biotechnology by Larry A. Sklar, Handbook of Flow Cytometry Methods by J. Paul Robinson, et al., Current Protocols in Cytometry, Wiley-Liss Pub, Flow Cytometry in Clinical Diagnosis, v4, (Carey, McCoy, and Keren, eds), ASCP Press, 2007, Ormerod, M. G. (ed.) (2000) Flow Cytometry—A practical approach. 3rd edition. Oxford University Press, Oxford, UK, Ormerod, M. G. (1999) Flow Cytometry. 2nd edition. BIOS Scientific Publishers, Oxford., and Flow Cytometry—A basic introduction. Michael G. Ormerod, 2008.

In some embodiments, cells are sorted by phenotype for further analysis. In some embodiments, cells of different phenotypes within the same cell composition are sorted by Fluorescence-activated cell sorting (FACS). FACS is a specialized type of flow cytometry that allows for sorting a heterogeneous mixture of cells into two or more containers, one cell at a time, based upon the specific light scattering and fluorescent characteristics of each cell. It is a useful scientific instrument as it provides fast, objective and quantitative recording of fluorescent signals from individual cells as well as physical separation of cells of particular interest.

In some embodiments, an input composition marker or T cell therapy marker can include any one or more of the markers of a cell composition, e.g., parameters or activities associated with an input cell composition or T cell therapy (e.g., CAR-T cells), respectively, described in published international applications WO 2019/032929, WO 2018/223101, WO 2019/089848, WO 2020/113194, WO 2019/090003, WO 2020/092848, WO 2019/113559, and WO 2018/157171, which are incorporated herein by reference in their entirety. In some embodiments, a subject marker can include any one or more of the features or characteristics of or associated with a subject (e.g., attributes of the subject or clinical attributes related to the subject in a clinical trial involving administration of the T cell therapy) described in published international applications WO 2019/032929, WO 2018/223101, WO 2019/089848, WO 2020/113194, WO 2019/090003, WO 2020/092848, WO 2019/113559, and WO 2018/157171, which are incorporated herein by reference in their entirety. In some embodiments, a clinical response to a T cell therapy (e.g., CAR-T cells) can include any one or more clinical response to a T cell therapy (e.g., CAR-T cells) described in published international applications WO 2019/032929, WO 2018/223101, WO 2019/089848, WO 2020/113194, WO 2019/090003, WO 2020/092848, WO 2019/113559, and WO 2018/157171, which are incorporated herein by reference in their entirety. Any one or more of such markers can be used as data to determine (e.g., predict) any one or more clinical responses in accord with the provided methods.

In some embodiments, the input composition markers are selected from the percentage of CD3+ cells in PBMCs isolated from leukapheresis, the CD4:CD8 ratio in the same PBMC population, the percentage of CD57+ cells in the same PBMC population, and the percentage of CD28+ cells in the same PBMC population.

In some embodiments, the process markers are early cell size, such as cell size on days 3, 4, or 5 of manufacturing, or the total nucleated cell (TNC yield). In some embodiments, the process marker is day 5 cell size.

In some embodiments, the drug product marker is selected from the percentage of CAR (CD3+ CAR+) cells in the T cell therapy, the total number of CAR+ cells (Total CAR yield), the vector copy number (VCN), and the CAR T cell potency.

In some embodiments, parameters of one or more subject markers are used for predicting if a subject will or will not exhibit a clinical response. In some embodiments, the one or more subject markers include subject fitness markers. In some embodiments, the one or more subject markers include subject immune profile markers. In some embodiments, the one or more subject markers include subject prior therapy markers. In some embodiments, the one or more subject markers include subject tumor burden markers.

In some embodiments, parameters of the subject markers are or can be obtained from medical records or the medical history of the subject. As an example, in some embodiments, parameters of certain subject fitness markers, such as age, or parameters of certain subject prior therapy markers, such as time since receiving certain prior therapies for treating the disease or condition, are or can be obtained from medical records or the medical history of the subject. In some embodiments, parameters for such subject markers are determined at the time or in relation to when the subject is being screened for manufacture and/or administration of the T cell therapy.

In some embodiments, parameters of the subject markers used for predicting clinical response are or can be measured using standard clinical assays. In some embodiments, the assays are or can be performed in a clinical setting.

In some embodiments, parameters of the subject markers used for predicting clinical response are or can be measured from a blood sample from the subject. As an example, in some embodiments, parameters of one or more subject markers, such as one or more subject fitness, tumor burden, or immune profile markers, are or can be measured from a blood sample collected from the subject. In some embodiments, the blood sample is or can be collected when the subject is being screened for manufacture and/or administration of the T cell therapy. In some embodiments, parameters of the subject markers that are measured from the blood sample are or can be measured when the subject is being screened for manufacture and/or administration of the T cell therapy. In some embodiments, the blood sample is a whole blood sample. In some embodiments, the blood sample is a serum sample. In some embodiments, the blood sample is a plasma sample. In some embodiments, parameters of one or more subject markers are measured from the blood sample using standard clinical assays.

Exemplary subject markers for predicting clinical response are provided below. Methods for measuring parameters of the subject markers can be identified and selected by one of ordinary skill in the art and can include standard clinical assays known in the art.

In some embodiments, the subject marker is a fitness marker. In some embodiments, the fitness marker is selected from age, body mass index (BMI), albumin blood sample level, alkaline phosphatase (ALP) blood sample level, aspartate aminotransferase blood sample (AST) level, alanine aminotransferase (ALT) blood sample level, direct bilirubin blood sample level, bilirubin blood sample level, creatinine blood sample level, and creatinine clearance. In some embodiments, the blood sample is a whole blood sample. In some embodiments, the blood sample is a serum sample. In some embodiments, the blood sample is a plasma sample.

In some embodiments, the subject marker is another clinical attribute and can include treatment history, disease burden (e.g., a measurement of tumor burden), expression of biomarkers and combinations of biomarkers, and combinations thereof. In some embodiments, treatment history includes prior therapy markers, such as time since diagnosis, number of prior regimens, time since prior corticosteroid therapy, time since prior alkylating agent therapy, time since prior topoisomerase inhibitor (TI) therapy, time since prior proteasome inhibitor (PI) therapy, or time since prior autologous stem cell transplant (ASCT) therapy.

In some embodiments, the subject marker is a tumor burden marker. In some embodiments, the tumor burden marker is selected from bone marrow plasma cell (BMPC) percentage, beta-2 microglobulin (B2M) blood sample level, immunoglobulin G blood sample level, lactase dehydrogenase (LDH) blood sample level, kappa:lambda light chain blood sample level, total immunoglobulin-free light chain blood sample level, mononuclear protein (M-protein) blood sample level, platelets blood sample count, sodium blood sample level, and blood sample B-cell maturation antigen (sBCMA) level. In some embodiments, the blood sample is a whole blood sample. In some embodiments, the blood sample is a serum sample. In some embodiments, the blood sample is a plasma sample.

In some embodiments, the subject marker is an immune profile marker. In some embodiments, the immune profile marker is selected from D-dimer blood sample level, fibrinogen blood sample level, lymphocytes blood sample count, monocyte blood sample count, monocyte:leukocyte (mono:leuk) ratio in blood sample, red blood cell (RBC) blood sample count, and white blood cell (WBC) blood sample count. In some embodiments, the blood sample is a whole blood sample. In some embodiments, the blood sample is a serum sample. In some embodiments, the blood sample is a plasma sample.

Non-limiting examples of subject markers, input composition markers, T cell therapy markers, e.g., drug product markers, and process markers used as data in the provided methods to determine (e.g., predict) clinical response or manufacturing success are described in the following subsections. In some aspects, the methods for predicting whether or not a T cell therapy will be therapeutically effective use any of the markers described herein. In some aspects, the methods for predicting whether or not a subject will exhibit a clinical response use any of the markers described herein.

The following sections also describe threshold values above or below which a marker is indicative of a subject being likely to exhibit a clinical response. In some aspects, such threshold values are also associated with markers indicating that a therapeutically effective T cell therapy can be generated for a subject.

### 1. Input Composition Markers

Various markers associated with the cells, e.g., PBMCs, isolated from an apheresis sample taken from a subject to be treated with a T cell therapy are contemplated for use according to the methods provided herein, e.g., methods of using thresholds of one or a combination of the markers or methods of machine learning. A subject to be treated with a T cell therapy may also be referred to herein as a patient.

In some embodiments, the input composition markers include the percentage of CD3+ cells in the PBMC population isolated from an apheresis sample from a patient. In some embodiments, a decreased percentage of CD3+ cells in the PBMC population is correlated with unfavorable patient outcome. In some embodiments, an increased percentage of CD3+ in the PBMC population is correlated with favorable patient outcome.

In some embodiments, the parameter of the percentage of CD3+ cells of a patient predicted not to exhibit the clinical response is lower than an associated threshold value. In some embodiments, the parameter of the percentage of CD3+ cells of a patient predicted to exhibit the clinical response is higher than the associated threshold value. In some embodiments, the associated threshold value is between or between about 2 percent and 40 percent. In some embodiments, the threshold is between or between about 2 percent and 36 percent, 2 percent and 32 percent, 2 percent and 28 percent, 2 percent and 24 percent, 2 percent and 20 percent, 2 percent and 16 percent, 2 percent and 12 percent, 2 percent and 8 percent, or 2 percent and 4 percent. In some embodiments, the threshold is between or between about 4 percent and 40 percent, 4 percent and 36 percent, 4 percent and 32 percent, 4 percent and 28 percent, 4 percent and 24 percent, 4 percent and 20 percent, 4 percent and 16 percent, 4 percent and 12 percent, or 4 percent and 8 percent. In some embodiments, the threshold is between or between about 8 percent or 40 percent, 8 percent and 36 percent, 8 percent and 32 percent, 8 percent and 28 percent, 8 percent and 24 percent, 8 percent and 20 percent, 8 percent and 16 percent, or 8 percent and 12 percent. In some embodiments, the threshold is between or between about 12 percent and 40 percent, 12 percent and 36 percent, 12 percent and 32 percent, 12 percent and 28 percent, 12 percent and 24 percent, 12 percent and 20 percent, or 12 percent and 16 percent. In some embodiments, the threshold is between or between about 16 percent and 40 percent, 16 percent and 36 percent, 16 percent and 32 percent, 16 percent and 28 percent, 16 percent and 24 percent, or 16 percent and 20 percent. In some embodiments, the threshold is between or between about 20 percent and 40 percent, 20 percent and 36 percent, 20 percent and 32 percent, 20 percent and 28 percent, or 20 percent and 24 percent. In some embodiments, the threshold is between or between about 24 percent and 40 percent, 24 percent and 36 percent, 24 percent and 32 percent, or 24 percent and 28 percent. In some embodiments, the threshold is between or between about 28 percent and 40 percent, 28 percent and 36 percent, or 28 percent and 32 percent. In some embodiments, the threshold is between or between about 32 percent and 40 percent or 32 percent and 36 percent. In some embodiments, the threshold is between or between about 36 percent and 40 percent. In some embodiments, the associated threshold value is between or between about 2 percent and 38 percent. In some embodiments, the associated threshold value is between or between about 11 percent and 26 percent. In some embodiments, the associated threshold value is or is about 16 percent.

In some embodiments, the input composition markers include the ratio of CD4+ to CD8+ cells in the PBMC population isolated from an apheresis sample from a patient. In some embodiments, a decreased CD4:CD8 ratio is correlated with unfavorable patient outcome. In some embodiments, an increased CD4:CD8 ratio is correlated with favorable patient outcome.

In some embodiments, the parameter of the ratio of CD4 to CD8 cells of a patient predicted not to exhibit the clinical response is lower than an associated threshold value. In some embodiments, the parameter of the parameter of the ratio of CD4 to CD8 cells of a patient predicted to exhibit the clinical response is higher than the associated threshold value. In some embodiments, the associated threshold value is between or between about 0.2 and 1.4. In some embodiments, the associated threshold value is between or between about 0.2 and 1.2, 0.2 and 1.0, 0.2 and 0.8, 0.2 and 0.6, or 0.2 and 0.4. In some embodiments, the associated threshold value is between or between about 0.4 and 1.4, 0.4 and 1.2, 0.4 and 1.0, 0.4 and 0.8, or 0.4 and 0.6. In some embodiments, the associated threshold value is between or between about 0.6 and 1.4, 0.6 and 1.2, 0.6 and 1.0, or 0.6 and 0.8. In some embodiments, the associated threshold value is between or between about 0.8 and 1.4, 0.8 and 1.2 or 0.8 and 1.0. In some embodiments, the associated threshold value is between or between about 1.0 and 1.4 or 1.0 and 1.2. In some embodiments, the associated threshold value is between or between about 1.2 and 1.4. In some embodiments, the associated threshold value is between 0.2 and 1.3. In some embodiments, the associated threshold value is between 0.4 and 0.6. In some embodiments, the associated threshold value is or is about 0.5.

In some embodiments, the input composition markers include the percentage of CD57+ cells in the PBMC population isolated from a leukapheresis sample from a patient. CD57 is a marker of cell senescence. In some embodiments, a decreased percentage of CD57+ cells is correlated with favorable patient outcome. In some embodiments, an increased percentage of CD57+ cells is correlated with unfavorable patient outcome.

In some embodiments, the parameter of the percentage of CD57+ cells of a patient predicted not to exhibit the clinical response is greater than or equal to an associated threshold value. In some embodiments, the parameter of the percentage of CD57+ cells of a patient predicted to exhibit the clinical response is less than or equal to than the associated threshold value. In some embodiments, the associated threshold value is between or between about 30 percent and 75 percent. In some embodiments, the associated threshold value is between or between about 30 percent and 60 percent or 30 percent and 45 percent. In some embodiments, the associated threshold value is between or between about 45 percent and 75 percent or 45 percent and 60 percent. In some embodiments, the associated threshold value is between or between about 60 percent and 75 percent. In some embodiments, the associated threshold value is between or between about 31 percent and 74 percent. In some embodiments, the associated threshold value is between or between about 45 percent and 65 percent. In some embodiments, the associated threshold value is or is about 50 percent.

In some embodiments, the input composition markers include the percentage of CD28+ cells in the PBMC population isolated from an apheresis sample from a patient. In some aspects, CD28 is a marker of less differentiation. CD28 (which is also known as T-cell-specific surface glycoprotein CD28 and TP44) is involved in T-cell activation, proliferation, cytokine production, and survival. In some embodiments, CD28 expression (e.g., the percentage of CD28+ cells or CD28+CD3+ cells) may identify cells with increased proliferative capacity. In some embodiments, a decreased percentage of CD28+ cells is correlated with unfavorable patient outcome. In some embodiments, an increased percentage of CD28+ cells is correlated with favorable patient outcome.

In some embodiments, the parameter of the percentage of CD28+ cells of a patient predicted not to exhibit the clinical response is lower than an associated threshold value. In some embodiments, the parameter of the percentage of CD28+ cells of a patient predicted to exhibit the clinical response is greater than the associated threshold value. In some embodiments, the associated threshold value is between or between about 50 percent and 90 percent. In some embodiments, the associated threshold value is between or between about 50 percent and 80 percent, 50 percent and 70 percent, or 50 percent and 60 percent. In some embodiments, the associated threshold value is between or between about 60 percent and 90 percent, 60 percent and 80 percent, or 60 percent and 70 percent. In some embodiments, the associated threshold value is between or between about 70 percent and 90 percent or 70 percent and 80 percent. In some embodiments, the associated threshold value is between or between about 50 percent and 87.5 percent. In some embodiments, the associated threshold value is between or between about 60 percent and 76 percent. In some embodiments, the associated threshold value is or is about 62.5 percent.

In some embodiments, higher quality PBMC phenotype is correlated with a decreased percentage of CD57+ cells and increased percentage of CD28+ cells. In some embodiments, lower quality PBMC phenotype is correlated with an increased percentage of CD57+ cells and decreased percentage of CD28+ cells.

### 2. Process Markers

Various markers associated with the manufacturing of T cells from the input composition containing cells, e.g., PBMCs, isolated from an apheresis sample taken from a subject to be treated with a T cell therapy are contemplated for use according to the methods provided herein, e.g., methods of using thresholds of one or a combination of the markers or methods of machine learning. A subject to be treated with a T cell therapy may also be referred to herein as a patient.

In some embodiments, the process markers include early cell size. In some embodiments, the early cell size can be a day 3 manufacturing, day 4 manufacturing, or day 5 manufacturing cell size. In some embodiments, the early cell size is the day 5 cell size. In some embodiments, the day 5 cell size is an indicator of activation. In some embodiments, smaller early cell size is correlated with unfavorable patient outcome. In some embodiments, larger early cell size is correlated with favorable patient outcome.

In some embodiments, the parameter of the day 5 cell size of a patient predicted not to exhibit the clinical response is lower than an associated threshold value. In some embodiments, the parameter of the day 5 cell size of a patient predicted to exhibit the clinical response is greater than the associated threshold value. In some embodiments, the associated threshold value is between or between about 400 μm³ and 800 μm³. In some embodiments, the associated threshold value is between or between about 400 μm³ and 700 μm³, 400 μm³ and 600 μm³, or 400 μm³ and 500 μm³. In some embodiments, the associated threshold value is between or between about 500 μm³ and 800 μm³, 500 μm³ and 700 μm³, or 500 μm³ and 600 μm³. In some embodiments, the associated threshold value is between or between about 600 μm³ and 800 μm³ or 600 μm³ and 700 μm³. In some embodiments, the associated threshold value is between or between about 700 μm³ and 800 μm³. In some embodiments, the associated threshold value is between or between about 600 μm³ and 780 μm³. In some embodiments, the associated threshold value is or is about 650 μm³.

In some embodiments, the process markers include total nucleated cell (TNC) yield. In some embodiments, a smaller TNC yield is correlated with unfavorable outcome. In some embodiments, a larger TNC yield is correlated with favorable patient outcome.

In some embodiments, the parameter of the TNC yield of a patient predicted not to exhibit the clinical response is lower than an associated threshold value. In some embodiments, the parameter of the TNC yield of a patient predicted to exhibit the clinical response is greater than the associated threshold value. In some embodiments, the associated threshold value is between or between about 1.5×10¹⁰ cells to 7.5×10¹⁰ cells. In some embodiments, the associated threshold value is between or between about 1.5×10¹⁰ cells to 6.0×10¹⁰ cells, 1.5×10¹⁰ cells to 4.5×10¹⁰ cells, or 1.5×10¹⁰ cells to 3.0×10¹⁰ cells. In some embodiments, the associated threshold value is between or between about 3.0×10¹⁰ cells to 7.5×10¹⁰ cells, 3.0×10¹⁰ cells to 6.0×10¹⁰ cells, or 3.0×10¹⁰ cells to 4.5×10¹⁰ cells. In some embodiments, the associated threshold value is between or between about 4.5×10¹⁰ cells to 7.5×10¹⁰ cells or 4.5×10¹⁰ cells to 6.0×10¹⁰ cells. In some embodiments, the associated threshold value is between or between about 1.5×10¹⁰ cells to 7.0×10¹⁰ cells. In some embodiments, the associated threshold value is between or between about 2.6×10¹⁰ cells to 4.5×10¹⁰ cells. In some embodiments, the associated threshold value is or is about 3.0×10¹⁰ cells.

### 3. Drug Product (DP) Markers

Various markers associated with the cells of the T cell therapy, e.g., drug product (DP) markers, that is manufactured from the PBMCs isolated from a apheresis sample taken from a subject to be treated with the T cell therapy are contemplated for use according to the methods provided herein, e.g., methods of using thresholds of one or a combination of the markers or methods of machine learning. A subject to be treated with a T cell therapy may also be referred to herein as a patient.

In some embodiments, the drug product markers include the percentage of CAR (CD3+ CAR+) cells. In some embodiments, decreased percentage of CAR cells is correlated with unfavorable outcome. In some embodiments, increased percentage of CAR cells is correlated with favorable patient outcome.

In some embodiments, the parameter of the percentage of CAR cells of a patient predicted not to exhibit the clinical response is lower than an associated threshold value. In some embodiments, the parameter of the percentage of CAR cells of a patient predicted to exhibit the clinical response is greater than the associated threshold value. In some embodiments, the associated threshold value is between or between about 30 percent and 55 percent. In some embodiments, the associated threshold value is between or between about 30 percent and 50 percent, 30 percent and 45 percent, 30 percent and 40 percent, or 30 percent and 35 percent. In some embodiments, the associated threshold value is between or between about 35 percent and 55 percent, 35 percent and 50 percent, 35 percent and 45 percent, or 35 percent and 40 percent. In some embodiments, the associated threshold value is between or between about 40 percent and 55 percent, 40 percent and 50 percent, or 40 percent and 45 percent. In some embodiments, the associated threshold value is between or between about 45 percent and 55 percent or 45 percent and 50 percent. In some embodiments, the associated threshold value is between or between about 50 percent and 55 percent. In some embodiments, the associated threshold value is between or between about 35 percent and 50 percent. In some embodiments, the associated threshold value is or is about 40 percent.

In some embodiments, the drug product markers include the total number of CAR+ cells (total CAR yield). In some embodiments, decreased total number of CAR+ cells is correlated with unfavorable outcome. In some embodiments, increased total number CAR+ cells is correlated with favorable patient outcome.

In some embodiments, the parameter of the total number of CAR+ cells of a patient predicted not to exhibit the clinical response is lower than an associated threshold value. In some embodiments, the parameter of the percentage of CAR cells of a patient predicted to exhibit the clinical response is greater than the associated threshold value. In some embodiments, the associated threshold value is between or between about 0.5×10⁹ cells and 2.0×10⁹ cells. In some embodiments, the associated threshold value is between or between about 0.5×10⁹ cells and 1.5×10⁹ cells or 0.5×10⁹ cells and 1.0×10⁹ cells. In some embodiments, the associated threshold value is between or between about 1.0×10⁹ cells and 2.0×10⁹ cells or 1.0×10⁹ cells and 1.5×10⁹ cells. In some embodiments, the associated threshold value is between or between about 1.0×10⁹ cells and 1.75×10⁹ cells. In some embodiments, the associated threshold value is or is about 1.5×10⁹ cells.

In some embodiments, the drug product markers include the vector copy number (VCN). In some embodiments, decreased VCN is correlated with unfavorable outcome. In some embodiments, increased VCN is correlated with favorable patient outcome.

In some embodiments, the parameter of the VCN of a patient predicted not to exhibit the clinical response is lower than an associated threshold value. In some embodiments, the parameter of the percentage of CAR cells of a patient predicted to exhibit the clinical response is greater than the associated threshold value. In some embodiments, the associated threshold value is between or between about 4.5 copies/μg DNA and 8.5 copies/μg DNA. In some embodiments, the associated threshold value is between or between about 4.5 copies/μg DNA and 7.5 copies/μg DNA, 4.5 copies/μg DNA and 6.5 copies/μg DNA or 4.5 copies/μg DNA and 5.5 copies/μg DNA. In some embodiments, the associated threshold value is between or between about 5.5 copies/μg DNA and 8.5 copies/μg DNA, 5.5 copies/μg DNA and 7.5 copies/μg DNA or 5.5 copies/μg DNA and 6.5 copies/μg DNA. In some embodiments, the associated threshold value is between or between about 6.5 copies/μg DNA and 8.5 copies/μg DNA or 6.5 copies/μg DNA and 7.5 copies/μg DNA. In some embodiments, the associated threshold value is between or between about 7.5 copies/μg DNA and 8.5 copies/μg DNA. In certain embodiments, the associated threshold value is between or between about 4.5 copies/μg DNA and 8 copies/μg DNA. In certain embodiments, the associated threshold value is between or between about 5 copies/μg DNA and 6.5 copies/μg DNA. In some embodiments, the associated threshold value is or is about 6 copies/μg DNA.

In some embodiments, the drug product markers include the T cell potency. In some embodiments, decreased potency is correlated with unfavorable outcome. In some embodiments, increased potency is correlated with favorable patient outcome.

### 4. Prior Therapies Markers

Various markers associated with the prior therapies received by the subject are contemplated for use according to the methods provided herein, e.g., methods of using thresholds of one or a combination of the markers or methods of machine learning. A subject to be treated with a T cell therapy may also be referred to herein as a patient.

Prior therapy markers can include the number of prior therapies. In some embodiments, a greater number of prior therapies is correlated with unfavorable patient outcome. In some embodiments, a fewer number of prior therapies is correlated with favorable patient outcome.

In some embodiments, the parameter of the number of prior therapies of a patient predicted not to exhibit the clinical response is greater than or equal to an associated threshold value. In some embodiments, the parameter of the number of prior therapies of a patient predicted to exhibit the clinical response is less than or equal to the associated threshold value. In some embodiments, the associated threshold value is between 4 therapies and 11 therapies. In some embodiments, the associated value is between 4 therapies and 10 therapies, 4 therapies and 9 therapies, 4 therapies and 8 therapies, 4 therapies and 7 therapies, 4 therapies and 6 therapies, or 4 therapies and 5 therapies. In some embodiments, the associated value is between 5 therapies and 11 therapies, 5 therapies and 10 therapies, 5 therapies and 9 therapies, 5 therapies and 8 therapies, 5 therapies and 7 therapies, or 5 therapies and 6 therapies. In some embodiments, the associated value is between 6 therapies and 11 therapies, 6 therapies and 10 therapies, 6 therapies and 9 therapies, 6 therapies and 8 therapies, or 6 therapies and 7 therapies. In some embodiments, the associated value is between 7 therapies and 11 therapies, 7 therapies and 10 therapies, 7 therapies and 9 therapies, or 7 therapies and 8 therapies. In some embodiments, the associated value is between 8 therapies and 11 therapies, 8 therapies and 10 therapies, or 8 therapies and 9 therapies. In some embodiments, the associated value is between 9 therapies and 11 therapies or 9 therapies and 10 therapies. In some embodiments, the associated value is between 10 therapies and 11 therapies. In some embodiments, the associated value is between 4 and 5 therapies. In some embodiments, the associated threshold value is 5 therapies.

Prior therapy markers can include the time since diagnosis. In some embodiments, a shorter time since diagnosis is correlated with unfavorable patient outcome. In some embodiments, a longer time since diagnosis is correlated with favorable patient outcome.

In some embodiments, the parameter of the time since diagnosis of a patient predicted not to exhibit the clinical response is greater than or equal to an associated threshold value. In some embodiments, the parameter of the time since diagnosis a patient predicted to exhibit the clinical response is less than or equal to the associated threshold value. In some embodiments, the associated threshold value is between 2 years and 10 years. In some embodiments, the associated threshold value is between 2 years and 9 years, 2 years and 8 years, 2 years and 7 years, 2 years and 6 years, 2 years and 5 years, 2 years and 4 years, or 2 years and 3 years. In some embodiments, the associated threshold value is between 3 years and 10 years, 3 years and 9 years, 3 years and 8 years, 3 years and 7 years, 3 years and 6 years, 3 years and 5 years, or 3 years and 4 years. In some embodiments, the associated threshold value is between 4 years and 10 years, 4 years and 9 years, 4 years and 8 years, 4 years and 7 years, 4 years and 6 years, or 4 years and 5 years. In some embodiments, the associated threshold value is between 5 years and 10 years, 5 years and 9 years, 5 years and 8 years, 5 years and 7 years, or 5 years and 6 years. In some embodiments, the associated threshold value is between 6 years and 10 years, 6 years and 9 years, 6 years and 8 years, or 6 years and 7 years. In some embodiments, the associated threshold value is between 7 years and 10 years, 7 years and 9 years, or 7 years and 8 years. In some embodiments, the associated threshold value is between 8 years and 10 years or 8 years and 9 years. In some embodiments, the associated threshold value is between 9 years and 10 years. In some embodiments, the associated threshold value is between or between about 2.2 years and 10 years. In some embodiments, the associated threshold value is between or between about 5.5 and 8.3 years. In some embodiments, the associated threshold value is or is about 7.5 years.

Prior therapy markers can include the time since prior therapy. Prior therapies can include prior autologous stem cell transplant (ASCT) therapy, prior alkylating agent therapy, prior topoisomerase inhibitor (TI) therapy, prior proteasome inhibitor (PI) therapy, or prior corticosteroid therapy. In some embodiments, more recent prior alkylating agent therapy, more recent prior TI therapy, or more recent prior PI therapy is correlated with unfavorable patient outcome. In some embodiments, more distant prior alkylating agent therapy, more recent prior TI therapy, or more recent prior PI therapy is correlated with favorable patient outcome.

In some embodiments, the parameter of the time since prior ASCT therapy of a patient predicted not to exhibit the clinical response is lower than an associated threshold value. In some embodiments, the parameter of the time since prior ASCT therapy of a patient predicted to exhibit the clinical response is greater than the associated threshold value. In some embodiments, the associated threshold value is between 25 days and 3225 days. In some embodiments, the associated threshold value is between 25 days and 3025 days, 25 days and 2825 days, 25 days and 2625 days, 25 days and 2425 days, 25 days and 2225 days, 25 days and 2025 days, 25 days and 1825 days, 25 days and 1625 days, 25 days and 1425 days, 25 days and 1225 days, 25 days and 1025 days, 25 days and 825 days, 25 days and 625 days, 25 days and 425 days, or 25 days and 225 days. In some embodiments, the associated threshold value is between 225 days and 3225 days, 225 days and 3025 days, 225 days and 2825 days, 225 days and 2625 days, 225 days and 2425 days, 225 days and 2225 days, 225 days and 2025 days, 225 days and 1825 days, 225 days and 1625 days, 225 days and 1425 days, 225 days and 1225 days, 225 days and 1025 days, 225 days and 825 days, 225 days and 625 days, or 225 days and 425 days. In some embodiments, the associated threshold value is between 425 days and 3225 days, 425 days and 3025 days, 425 days and 2825 days, 425 days and 2625 days, 425 days and 2425 days, 425 days and 2225 days, 425 days and 2025 days, 425 days and 1825 days, 425 days and 1625 days, 425 days and 1425 days, 425 days and 1225 days, 425 days and 1025 days, 425 days and 825 days, or 425 days and 625 days. In some embodiments, the associated threshold value is between 625 days and 3225 days, 625 days and 3025 days, 625 days and 2825 days, 625 days and 2625 days, 625 days and 2425 days, 625 days and 2225 days, 625 days and 2025 days, 625 days and 1825 days, 625 days and 1625 days, 625 days and 1425 days, 625 days and 1225 days, 625 days and 1025 days, or 625 days and 825 days. In some embodiments, the associated threshold value is between 825 days and 3225 days, 825 days and 3025 days, 825 days and 2825 days, 825 days and 2625 days, 825 days and 2425 days, 825 days and 2225 days, 825 days and 2025 days, 825 days and 1825 days, 825 days and 1625 days, 825 days and 1425 days, 825 days and 1225 days, or 825 days and 1025 days. In some embodiments, the associated threshold value is between 1025 days and 3225 days, 1025 days and 3025 days, 1025 days and 2825 days, 1025 days and 2625 days, 1025 days and 2425 days, 1025 days and 2225 days, 1025 days and 2025 days, 1025 days and 1825 days, 1025 days and 1625 days, 1025 days and 1425 days, or 1025 days and 1225 days. In some embodiments, the associated threshold value is between 1225 days and 3225 days, 1225 days and 3025 days, 1225 days and 2825 days, 1225 days and 2625 days, 1225 days and 2425 days, 1225 days and 2225 days, 1225 days and 2025 days, 1225 days and 1825 days, 1225 days and 1625 days, or 1225 days and 1425 days. In some embodiments, the associated threshold value is between 1425 days and 3225 days, 1425 days and 3025 days, 1425 days and 2825 days, 1425 days and 2625 days, 1425 days and 2425 days, 1425 days and 2225 days, 1425 days and 2025 days, 1425 days and 1825 days, or 1425 days and 1625 days. In some embodiments, the associated threshold value is between 1625 days and 3225 days, 1625 days and 3025 days, 1625 days and 2825 days, 1625 days and 2625 days, 1625 days and 2425 days, 1625 days and 2225 days, 1625 days and 2025 days, or 1625 days and 1825 days. In some embodiments, the associated threshold value is between 1825 days and 3225 days, 1825 days and 3025 days, 1825 days and 2825 days, 1825 days and 2625 days, 1825 days and 2425 days, 1825 days and 2225 days, or 1825 days and 2025 days. In some embodiments, the associated threshold value is between 2025 days and 3225 days, 2025 days and 3025 days, 2025 days and 2825 days, 2025 days and 2625 days, 2025 days and 2425 days, or 2025 days and 2225 days. In some embodiments, the associated threshold value is between 2225 days and 3225 days, 2225 days and 3025 days, 2225 days and 2825 days, 2225 days and 2625 days, or 2225 days and 2425 days. In some embodiments, the associated threshold value is between 2425 days and 3225 days, 2425 days and 3025 days, 2425 days and 2825 days, or 2425 days and 2625 days. In some embodiments, the associated threshold value is between 2625 days and 3225 days, 2625 days and 3025 days, or 2625 days and 2825 days. In some embodiments, the associated threshold value is between 2825 days and 3225 days or 2825 days and 3025 days. In some embodiments, the associated threshold value is between 3025 days and 3225 days. In some embodiments, the associated threshold value is between 26 days and 3205 days. In some embodiments, the associated threshold value is between 641 days and 2941 days. In some embodiments, the associated threshold value is or is about 1351 days.

In some embodiments, the parameter of the time since prior alkylating agent therapy of a patient predicted not to exhibit the clinical response is lower than an associated threshold value. In some embodiments, the parameter of the time since prior alkylating agent therapy of a patient predicted to exhibit the clinical response is greater than the associated threshold value. In some embodiments, the associated threshold value is between 10 days and 510 days. In some embodiments, the associated threshold value is between 10 days and 460 days, 10 days and 410 days, 10 days and 360 days, 10 days and 310 days, 10 days and 260 days, 10 days and 210 days, 10 days and 160 days, 10 days and 110 days, or 10 days 60 days. In some embodiments, the associated threshold value is between 60 days and 510 days, 60 days and 460 days, 60 days and 410 days, 60 days and 360 days, 60 days and 310 days, 60 days and 260 days, 60 days and 210 days, 60 days and 160 days, or 60 days and 110 days. In some embodiments, the associated threshold value is between 110 days and 510 days, 110 days and 460 days, 110 days and 410 days, 110 days and 360 days, 110 days and 310 days, 110 days and 260 days, 110 days and 210 days, or 110 days and 160 days. In some embodiments, the associated threshold value is between 160 days and 510 days, 160 days and 460 days, 160 days and 410 days, 160 days and 360 days, 160 days and 310 days, 160 days and 260 days, or 160 days and 210 days. In some embodiments, the associated threshold value is between 210 days and 510 days, 210 days and 460 days, 210 days and 410 days, 210 days and 360 days, 210 days and 310 days, or 210 days and 260 days. In some embodiments, the associated threshold value is between 260 days and 510 days, 260 days and 460 days, 260 days and 410 days, 260 days and 360 days, or 260 days and 310 days. In some embodiments, the associated threshold value is between 310 days and 510 days, 310 days and 460 days, 310 days and 410 days, or 310 days and 360 days. In some embodiments, the associated threshold value is between 360 days and 510 days, 360 days and 460 days, or 360 days and 410 days. In some embodiments, the associated threshold value is between 410 days and 510 days or 460 days and 460 days. In some embodiments, the associated threshold value is between 460 days and 510 days. In some embodiments, the associated threshold value is between 11 days and 493 days. In some embodiments, the associated threshold value is between 230 days and 244 days. In some embodiments, the associated threshold value is or is about 244 days.

In some embodiments, the parameter of the time since prior topoisomerase inhibitor therapy of a patient predicted not to exhibit the clinical response is lower than an associated threshold value. In some embodiments, the parameter of the time since prior topoisomerase inhibitor therapy of a patient predicted to exhibit the clinical response is greater than the associated threshold value. In some embodiments, the associated threshold value is between 25 days and 3425 days. In some embodiments, the associated threshold value is between 25 days and 3225 days, 25 days and 3025 days, 25 days and 3025 days, 25 days and 2825 days, 25 days and 2625 days, 25 days and 2425 days, 25 days and 2225 days, 25 days and 2025 days, 25 days and 1825 days, 25 days and 1625 days, 25 days and 1425 days, 25 days and 1225 days, 25 days and 1025 days, 25 days and 825 days, 25 days and 625 days, 25 days and 425 days, or 25 days and 225 days. In some embodiments, the associated threshold value is between 225 days and 3425 days, 225 days and 3225 days, 225 days and 3025 days, 225 days and 2825 days, 225 days and 2625 days, 225 days and 2425 days, 225 days and 2225 days, 225 days and 2025 days, 225 days and 1825 days, 225 days and 1625 days, 225 days and 1425 days, 225 days and 1225 days, 225 days and 1025 days, 225 days and 825 days, 225 days and 625 days, or 225 days and 425 days. In some embodiments, the associated threshold value is between 425 days and 3425 days, 425 days and 3225 days, 425 days and 3025 days, 425 days and 2825 days, 425 days and 2625 days, 425 days and 2425 days, 425 days and 2225 days, 425 days and 2025 days, 425 days and 1825 days, 425 days and 1625 days, 425 days and 1425 days, 425 days and 1225 days, 425 days and 1025 days, 425 days and 825 days, or 425 days and 625 days. In some embodiments, the associated threshold value is between 625 days and 3425 days, 625 days and 3225 days, 625 days and 3025 days, 625 days and 2825 days, 625 days and 2625 days, 625 days and 2425 days, 625 days and 2225 days, 625 days and 2025 days, 625 days and 1825 days, 625 days and 1625 days, 625 days and 1425 days, 625 days and 1225 days, 625 days and 1025 days, or 625 days and 825 days. In some embodiments, the associated threshold value is between 825 days and 3425 days, 825 days and 3225 days, 825 days and 3025 days, 825 days and 2825 days, 825 days and 2625 days, 825 days and 2425 days, 825 days and 2225 days, 825 days and 2025 days, 825 days and 1825 days, 825 days and 1625 days, 825 days and 1425 days, 825 days and 1225 days, or 825 days and 1025 days. In some embodiments, the associated threshold value is between 1025 days and 3425 days, 1025 days and 3225 days, 1025 days and 3025 days, 1025 days and 2825 days, 1025 days and 2625 days, 1025 days and 2425 days, 1025 days and 2225 days, 1025 days and 2025 days, 1025 days and 1825 days, 1025 days and 1625 days, 1025 days and 1425 days, or 1025 days and 1225 days. In some embodiments, the associated threshold value is between 1225 days and 3425 days, 1225 days and 3225 days, 1225 days and 3025 days, 1225 days and 2825 days, 1225 days and 2625 days, 1225 days and 2425 days, 1225 days and 2225 days, 1225 days and 2025 days, 1225 days and 1825 days, 1225 days and 1625 days, or 1225 days and 1425 days. In some embodiments, the associated threshold value is between 1425 days and 3425 days, 1425 days and 3225 days, 1425 days and 3025 days, 1425 days and 2825 days, 1425 days and 2625 days, 1425 days and 2425 days, 1425 days and 2225 days, 1425 days and 2025 days, 1425 days and 1825 days, or 1425 days and 1625 days. In some embodiments, the associated threshold value is between 1625 days and 3425 days, 1625 days and 3225 days, 1625 days and 3025 days, 1625 days and 2825 days, 1625 days and 2625 days, 1625 days and 2425 days, 1625 days and 2225 days, 1625 days and 2025 days, or 1625 days and 1825 days. In some embodiments, the associated threshold value is between 1825 days and 3425 days, 1825 days and 3225 days, 1825 days and 3025 days, 1825 days and 2825 days, 1825 days and 2625 days, 1825 days and 2425 days, 1825 days and 2225 days, or 1825 days and 2025 days. In some embodiments, the associated threshold value is between 2025 days and 3425 days, 2025 days and 3225 days, 2025 days and 3025 days, 2025 days and 2825 days, 2025 days and 2625 days, 2025 days and 2425 days, or 2025 days and 2225 days. In some embodiments, the associated threshold value is between 2225 days and 3425 days, 2225 days and 3225 days, 2225 days and 3025 days, 2225 days and 2825 days, 2225 days and 2625 days, or 2225 days and 2425 days. In some embodiments, the associated threshold value is between 2425 days and 3425 days, 2425 days and 3225 days, 2425 days and 3025 days, 2425 days and 2825 days, or 2425 days and 2625 days. In some embodiments, the associated threshold value is between 2625 days and 3425 days, 2625 days and 3225 days, 2625 days and 3025 days, or 2625 days and 2825 days. In some embodiments, the associated threshold value is between 2825 days and 3425 days, 2825 days and 3225 days, or 2825 days and 3025 days. In some embodiments, the associated threshold value is between 3025 days and 3425 days or 3025 days and 3225 days. In some embodiments, the associated threshold value is between 3025 days and 3425 days. In some embodiments, the associated threshold value is between 87 days and 3356 days. In some embodiments, the associated threshold value is between 474 days and 676 days. In some embodiments, the associated threshold value is or is about 676 days.

In some embodiments, the parameter of the time since prior proteasome inhibitor therapy of a patient predicted not to exhibit the clinical response is lower than an associated threshold value. In some embodiments, the parameter of the time since prior proteasome inhibitor therapy of a patient predicted to exhibit the clinical response is greater than the associated threshold value. In some embodiments, the associated threshold value is between 10 days and 660 days. In some embodiments, the associated threshold value is between 10 days and 610 days, 10 days and 560 days, 10 days and 510 days, 10 days and 460 days, 10 days and 410 days, 10 days and 360 days, 10 days and 310 days, 10 days and 260 days, 10 days and 210 days, 10 days and 160 days, 10 days and 110 days, or 10 days 60 days. In some embodiments, the associated threshold value is between 60 days and 660 days, 60 days and 610 days, 60 days and 560 days, 60 days and 510 days, 60 days and 460 days, 60 days and 410 days, 60 days and 360 days, 60 days and 310 days, 60 days and 260 days, 60 days and 210 days, 60 days and 160 days, or 60 days and 110 days. In some embodiments, the associated threshold value is between 110 days and 660 days, 110 days and 610 days, 110 days and 560 days, 110 days and 510 days, 110 days and 460 days, 110 days and 410 days, 110 days and 360 days, 110 days and 310 days, 110 days and 260 days, 110 days and 210 days, or 110 days and 160 days. In some embodiments, the associated threshold value is between 160 days and 660 days, 160 days and 610 days, 160 days and 560 days, 160 days and 510 days, 160 days and 460 days, 160 days and 410 days, 160 days and 360 days, 160 days and 310 days, 160 days and 260 days, or 160 days and 210 days. In some embodiments, the associated threshold value is between 210 days and 660 days, 210 days and 610 days, 210 days and 560 days, 210 days and 510 days, 210 days and 460 days, 210 days and 410 days, 210 days and 360 days, 210 days and 310 days, or 210 days and 260 days. In some embodiments, the associated threshold value is between 260 days and 660 days, 260 days and 610 days, 260 days and 560 days, 260 days and 510 days, 260 days and 460 days, 260 days and 410 days, 260 days and 360 days, or 260 days and 310 days. In some embodiments, the associated threshold value is between 310 days and 660 days, 310 days and 610 days, 310 days and 560 days, 310 days and 510 days, 310 days and 460 days, 310 days and 410 days, or 310 days and 360 days. In some embodiments, the associated threshold value is between 360 days and 660 days, 360 days and 610 days, 360 days and 560 days, 360 days and 510 days, 360 days and 460 days, or 360 days and 410 days. In some embodiments, the associated threshold value is between 410 days and 660 days, 410 days and 610 days, 410 days and 560 days, 410 days and 510 days, or 460 days and 460 days. In some embodiments, the associated threshold value is between 460 days and 660 days, 460 days and 610 days, 460 days and 560 days, or 460 days and 510 days. In some embodiments, the associated threshold value is between 510 days and 660 days, 510 days and 610 days, or 510 days and 560 days. In some embodiments, the associated threshold value is between 560 days and 660 days or 560 days and 610 days. In some embodiments, the associated threshold value is between 610 days and 660 days. In some embodiments, the associated threshold value is between 11 days and 658 days. In some embodiments, the associated threshold value is between 51 days and 170 days. In some embodiments, the associated threshold value is or is about 87 days.

In some embodiments, the parameter of the time since prior corticosteroid therapy of a patient predicted not to exhibit the clinical response is lower than an associated threshold value. In some embodiments, the parameter of the time since prior corticosteroid therapy of a patient predicted to exhibit the clinical response is greater than the associated threshold value. In some embodiments, the associated threshold value is between 1 day and 2425 days. In some embodiments, the associated threshold value is between 1 day and 2225 days, 1 day and 2025 days, 1 day and 1825 days, 1 day and 1625 days, 1 day and 1425 days, 1 day and 1225 days, 1 day and 1025 days, 1 day and 825 days, 1 day and 625 days, 1 day and 425 days, or 1 day and 225 days. In some embodiments, the associated threshold value is between 25 days and 2425 days, 25 days and 2225 days, 25 days and 2025 days, 25 days and 1825 days, 25 days and 1625 days, 25 days and 1425 days, 25 days and 1225 days, 25 days and 1025 days, 25 days and 825 days, 25 days and 625 days, 25 days and 425 days, or 25 days and 225 days. In some embodiments, the associated threshold value is between 225 days and 2425 days, 225 days and 2225 days, 225 days and 2025 days, 225 days and 1825 days, 225 days and 1625 days, 225 days and 1425 days, 225 days and 1225 days, 225 days and 1025 days, 225 days and 825 days, 225 days and 625 days, or 225 days and 425 days. In some embodiments, the associated threshold value is between 425 days and 2425 days, 425 days and 2225 days, 425 days and 2025 days, 425 days and 1825 days, 425 days and 1625 days, 425 days and 1425 days, 425 days and 1225 days, 425 days and 1025 days, 425 days and 825 days, or 425 days and 625 days. In some embodiments, the associated threshold value is between 625 days and 2425 days, 625 days and 2225 days, 625 days and 2025 days, 625 days and 1825 days, 625 days and 1625 days, 625 days and 1425 days, 625 days and 1225 days, 625 days and 1025 days, or 625 days and 825 days. In some embodiments, the associated threshold value is between 825 days and 2425 days, 825 days and 2225 days, 825 days and 2025 days, 825 days and 1825 days, 825 days and 1625 days, 825 days and 1425 days, 825 days and 1225 days, or 825 days and 1025 days. In some embodiments, the associated threshold value is between 1025 days and 2425 days, 1025 days and 2225 days, 1025 days and 2025 days, 1025 days and 1825 days, 1025 days and 1625 days, 1025 days and 1425 days, or 1025 days and 1225 days. In some embodiments, the associated threshold value is between 1225 days and 2425 days, 1225 days and 2225 days, 1225 days and 2025 days, 1225 days and 1825 days, 1225 days and 1625 days, or 1225 days and 1425 days. In some embodiments, the associated threshold value is between 1425 days and 2425 days, 1425 days and 2225 days, 1425 days and 2025 days, 1425 days and 1825 days, or 1425 days and 1625 days. In some embodiments, the associated threshold value is between 1625 days and 2425 days, 1625 days and 2225 days, 1625 days and 2025 days, or 1625 days and 1825 days. In some embodiments, the associated threshold value is between 1825 days and 2425 days, 1825 days and 2225 days, or 1825 days and 2025 days. In some embodiments, the associated threshold value is between 2025 days and 2425 days or 2025 days and 2225 days. In some embodiments, the associated threshold value is between 2225 days and 2425 days. In some embodiments, the associated threshold value is between 12 days and 2257 days. In some embodiments, the associated threshold value is between 42 days and 59 days. In some embodiments, the associated threshold value is or is about 55 days.

### 5. Tumor Burden Markers

Various markers associated with the tumor burden of the subject are contemplated for use according to the methods provided herein, e.g., methods of using thresholds of one or a combination of the markers or methods of machine learning. A subject to be treated with a T cell therapy may also be referred to herein as a patient.

In some embodiments, the tumor burden markers include the blood sample level of lactate dehydrogenase (LDH). In some embodiments, increased blood sample LDH level is correlated with unfavorable patient outcome. In some embodiments, decreased blood sample LDH level is correlated with favorable patient outcome.

In some embodiments, the parameter of the blood sample LDH level of a patient predicted not to exhibit the clinical response is lower than an associated threshold value. In some embodiments, the parameter of the blood sample LDH level of a patient predicted to exhibit the clinical response is greater than the associated threshold value. In some embodiments, the associated threshold value is between or between about 150 U/L and 350 U/L. In some embodiments, the associated threshold value is between or between about 150 U/L and 300 U/L, 150 U/L and 250 U/L, or 150 U/L and 200 U/L. In some embodiments, the associated threshold value is between or between about 200 U/L and 350 U/L, 200 U/L and 300 U/L, 200 or U/L and 250 U/L. In some embodiments, the associated threshold value is between or between about 250 U/L and 350 U/L or 250 U/L and 300 U/L. In some embodiments, the associated threshold value is between or between about 300 U/L and 350 U/L. In some embodiments, the associated threshold value is between or between about 300 U/L and 319 U/L. In some embodiments, the associated threshold value is between or between about 131 U/L and 319 U/L. In some embodiments, the associated threshold value is or is about 246 U/L.

In some embodiments, the tumor burden markers include the ratio of kappa light chain to lambda light chain. In some embodiments, the parameter of the ratio of kappa light chain to lambda light chain of a patient predicted not to exhibit the clinical response is lower than an associated threshold value. In some embodiments, the parameter the ratio of kappa light chain to lambda light chain of a patient predicted to exhibit the clinical response is greater than the associated threshold value. In some embodiments, the associated threshold value is between or between about 0.003 and 800. In some embodiments, the associated threshold value is between or between about 0.003 and 700, 0.003 and 600, 0.003 and 500, 0.003 and 400, 0.003 and 300, 0.003 and 200, 0.003 and 100, or 0.003 and 50. In some embodiments, the associated threshold value is between or between about 1 and 800, land 700, 1 and 600, 1 and 500, 1 and 400, 1 and 300, 1 and 200, 1 and 100, or 1 and 50. In some embodiments, the associated threshold value is between or between about 50 and 800, 50 and 700, 50 and 600, 50 and 500, 50 and 400, 50 and 300, 50 and 200, or 50 and 100. In some embodiments, the associated threshold value is between or between about 100 and 800, 100 and 700, 100 and 600, 100 and 500, 100 and 400, 100 and 300, or 100 and 200. In some embodiments, the associated threshold value is between or between about 200 and 800, 200 and 700, 200 and 600, 200 and 500, 200 and 400, or 200 and 300. In some embodiments, the associated threshold value is between or between about 300 and 800, 300 and 700, 300 and 600, 300 and 500, or 300 and 400. In some embodiments, the associated threshold value is between or between about 400 and 800, 400 and 700, 400 and 600, or 400 and 500. In some embodiments, the associated threshold value is between or between about 500 and 800, 500 and 700, or 500 and 600. In some embodiments, the associated threshold value is between or between about 600 and 800 or 600 and 700. In some embodiments, the associated threshold value is between or between about 700 and 800. In some embodiments, the associated threshold value is between or between about 0.003 and 763. In some embodiments, the associated threshold value is between or between about 8.7 and 211. In some embodiments, the associated threshold value is 18.

In some embodiments, the tumor burden markers include the blood sample platelets count. In some embodiments, decreased blood sample platelets count is correlated with unfavorable outcome. In some embodiments, increased blood sample platelets count is correlated with favorable patient outcome.

In some embodiments, the parameter of the total number of blood sample platelets count of a patient predicted not to exhibit the clinical response is lower than an associated threshold value. In some embodiments, the parameter of the percentage of CAR cells of a patient predicted to exhibit the clinical response is greater than the associated threshold value. In some embodiments, the associated threshold value is between or between about 50×10⁹ platelets and 250×10⁹ platelets. In some embodiments, the associated threshold value is between or between about 50×10⁹ platelets and 200×10⁹ platelets, 50×10⁹ platelets and 150×10⁹ platelets, or 50×10⁹ platelets and 100×10⁹ platelets. In some embodiments, the associated threshold value is between or between about 100×10⁹ platelets and 250×10⁹ platelets, 100×10⁹ platelets and 200×10⁹ platelets, or 100×10⁹ platelets and 150×10⁹ platelets. In some embodiments, the associated threshold value is between or between about 150×10⁹ platelets and 250×10⁹ platelets or 150×10⁹ platelets and 200×10⁹ platelets. In some embodiments, the associated threshold value is between or between about 200×10⁹ platelets and 250×10⁹ platelets. In some embodiments, the associated threshold value is between or between about 53×10⁹ platelets and 212×10⁹ platelets. In some embodiments, the associated threshold value is between or between about 156×10⁹ platelets and 181×10⁹ platelets. In some embodiments, the associated threshold value is 181×10⁹ platelets.

In some embodiments, the tumor burden markers include the sodium concentration. In some embodiments, the parameter of the sodium concentration count of a patient predicted not to exhibit the clinical response is lower than an associated threshold value. In some embodiments, the parameter of the sodium concentration of a patient predicted to exhibit the clinical response is greater than the associated threshold value. In some embodiments, the associated threshold value is between or between about 132 mmol/L and 142 mml/L. In some embodiments, the associated threshold value is between or between about 132 mmol/L and 140 mml/L, 132 mmol/L and 138 mmol/L, 132 mmol/L and 136 mmol/L, or 132 mmol/L and 134 mmol/L. In some embodiments, the associated threshold value is between or between about 134 mmol/L and 140 mml/L, 134 mmol/L and 138 mmol/L, or 134 mmol/L and 136 mmol/L. In some embodiments, the associated threshold value is between or between about 136 mmol/L and 140 mml/L and 136 mmol/L and 138 mmol/L. In some embodiments, the associated threshold value is between or between about 138 mmol/L and 140 mml/L. In some embodiments, the associated threshold value is between or between about 132 mmol/L and 141 mml/L. In some embodiments, the associated threshold value is between or between about 136 mmol/L and 138 mml/L. In some embodiments, the associated threshold value is or is about 138 mml/L.

In some embodiments, the tumor burden markers include the bone marrow plasma cell (BMPC) percentage. In some embodiments, the parameter of the BMPC percentage of a patient predicted not to exhibit the clinical response is greater than or equal to an associated threshold value. In some embodiments, the parameter of the blood sample LDH level of a patient predicted to exhibit the clinical response is less than or equal to associated threshold value. In some embodiments, the associated threshold value is between or between about 20 percent and 100 percent. In some embodiments, the associated threshold value is between or between about 20 percent and 80 percent, 20 percent and 60 percent, or 20 percent and 40 percent. In some embodiments, the associated threshold value is between or between about 40 percent and 80 percent or 40 percent and 60 percent. In some embodiments, the associated threshold value is between or between about 60 percent and 80 percent. In some embodiments, the associated threshold value is between or between about 21 percent and 100 percent. In some embodiments, the associated threshold value is between or between about 56 percent and 80 percent. In some embodiments, the associated threshold value is or is about 56 percent.

In some embodiments, the tumor burden markers include the blood sample beta-2 microglobulin (B2M) level. In some embodiments, increased blood sample B2M level is correlated with unfavorable outcome. In some embodiments, decreased blood sample B2M level is correlated with favorable patient outcome.

In some embodiments, the parameter of the blood sample B2M level of a patient predicted not to exhibit the clinical response is greater than or equal to an associated threshold value. In some embodiments, the parameter of the blood sample B2M level of a patient predicted to exhibit the clinical response is less than or equal to the associated threshold value. In some embodiments, the associated threshold value is between or between about 2.7 mg/L and 7.7 mg/L. In some embodiments, the associated threshold value is between or between about 2.7 mg/L and 6.7 mg/L, 2.7 mg/L and 5.7 mg/L, 2.7 mg/L and 4.7 mg/L, or 2.7 mg/L and 3.7 mg/L. In some embodiments, the associated threshold value is between or between about 3.7 mg/L and 7.7 mg/L, 3.7 and 6.7 mg/L, 3.7 mg/L and 5.7 mg/L, or 3.7 mg/L and 4.7 mg/L. In some embodiments, the associated threshold value is between or between about 4.7 mg/L and 7.7 mg/L, 4.7 and 6.7 mg/L, or 4.7 mg/L and 5.7 mg/L. In some embodiments, the associated threshold value is between or between about 5.7 mg/L and 7.7 mg/L or 5.7 and 6.7 mg/L. In some embodiments, the associated threshold value is between or between about 3.2 mg/L and 4.6 mg/L. In some embodiments, the associated threshold value is 3.7 mg/L.

In some embodiments, the tumor burden markers include the blood sample immunoglobulin G (IgG) level. In some embodiments, increased blood sample IgG is correlated with unfavorable outcome. In some embodiments, decreased blood sample IgG level is correlated with favorable patient outcome.

In some embodiments, the parameter of the blood sample IgG level of a patient predicted not to exhibit the clinical response is greater than or equal to an associated threshold value. In some embodiments, the parameter of the blood sample IgG level of a patient predicted to exhibit the clinical response is less than or equal to the associated threshold value. In some embodiments, the associated threshold value is between or between about 2 g/L and 82 g/L. In some embodiments, the associated threshold value is between or between about 2 g/L and 72 g/L, 2 g/L and 62 g/L, 2 g/L and 52 g/L, 2 g/L and 42 g/L, 2 g/L and 32 g/L, 2 g/L and 22 g/L, or 2 g/L and 12 g/L. In some embodiments, the associated threshold value is between or between about 12 g/L and 82 g/L, 12 g/L and 72 g/L, 12 g/L and 62 g/L, 12 g/L and 52 g/L, 12 g/L and 42 g/L, 12 g/L and 32 g/L, or 12 g/L and 22 g/L. In some embodiments, the associated threshold value is between or between about 22 g/L and 82 g/L, 22 g/L and 72 g/L, 22 g/L and 62 g/L, 22 g/L and 52 g/L, 22 g/L and 42 g/L, or 22 g/L and 32 g/L. In some embodiments, the associated threshold value is between or between about 32 g/L and 82 g/L, 32 g/L and 72 g/L, 32 g/L and 62 g/L, 32 g/L and 52 g/L, or 32 g/L and 42 g/L. In some embodiments, the associated threshold value is between or between about 42 g/L and 82 g/L, 42 g/L and 72 g/L, 42 g/L and 62 g/L, or 42 g/L and 52 g/L. In some embodiments, the associated threshold value is between or between about 52 g/L and 82 g/L, 52 g/L and 72 g/L, or 52 g/L and 62 g/L. In some embodiments, the associated threshold value is between or between about 62 g/L and 82 g/L or 62 g/L and 72 g/L. In some embodiments, the associated threshold value is between or between about 72 g/L and 82 g/L. In some embodiments, the associated threshold value is between or between about 2.8 g/L and 75 g/L. In some embodiments, the associated threshold value is between or between about 14 g/L and 35 g/L. In some embodiments, the associated threshold value is or is about 17 g/L.

In some embodiments, the tumor burden markers include the free light chain total concentration. In some embodiments, the parameter of the free light chain total concentration of a patient predicted not to exhibit the clinical response is greater than or equal to an associated threshold value. In some embodiments, the parameter of the free light chain total concentration a patient predicted to exhibit the clinical response is less than or equal to the associated threshold value. In some embodiments, the associated threshold value is between or between about 0.008 g/L and 12 g/L. In some embodiments, the associated threshold value is between or between about 0.08 g/L and 12 g/L, 0.08 g/L and 8 g/L, 0.08 g/L and 4 g/L, 0.08 g/L and 2 g/L, or 0.08 g/L and 0.8 g/L. In some embodiments, the associated threshold value is between or between about 0.8 g/L and 12 g/L, 0.8 g/L and 8 g/L, 0.8 g/L and 4 g/L, or 0.8 g/L and 2 g/L. In some embodiments, the associated threshold value is between or between about 2 g/L and 12 g/L, 2 g/L and 8 g/L, or 2 g/L and 4 g/L. In some embodiments, the associated threshold value is between or between about 4 g/L and 12 g/L or 4 g/L and 8 g/L. In some embodiments, the associated threshold value is between or between about 8 g/L and 12 g/L. In some embodiments, the associated threshold value is between or between about 0.2 g/L and 1.0 g/L. In some embodiments, the associated threshold value is or is about 0.1 g/L.

In some embodiments, the tumor burden markers include the blood sample mononuclear protein (M-protein) level. In some embodiments, increased blood sample M-protein level is correlated with unfavorable patient outcome. In some embodiments, decreased blood sample M-protein level is correlated with favorable patient outcome.

In some embodiments, the parameter of the blood sample M-protein level of a patient predicted not to exhibit the clinical response is greater than or equal to an associated threshold value. In some embodiments, the parameter of the blood sample M-protein level of a patient predicted to exhibit the clinical response is less than or equal to the associated threshold value. In some embodiments, the associated threshold value is between or between about 4 g/L and 32 g/L. In some embodiments, the associated threshold value is between or between about 4 g/L and 28 g/L, 4 g/L and 24 g/L, 4 g/L and 20 g/L, 4 g/L and 16 g/L, 4 g/L and 12 g/L, or 4 g/L and 8 g/L. In some embodiments, the associated threshold value is between or between about 8 g/L and 32 g/L, 8 g/L and 28 g/L, 8 g/L and 24 g/L, 8 g/L and 20 g/L, 8 g/L and 16 g/L, or 8 g/L and 12 g/L. In some embodiments, the associated threshold value is between or between about 12 g/L and 32 g/L, 12 g/L and 28 g/L, 12 g/L and 24 g/L, 12 g/L and 20 g/L, or 12 g/L and 16 g/L. In some embodiments, the associated threshold value is between or between about 16 g/L and 32 g/L, 16 g/L and 28 g/L, 16 g/L and 24 g/L, or 16 g/L and 20 g/L. In some embodiments, the associated threshold value is between or between about 20 g/L and 32 g/L, 20 g/L and 28 g/L, or 20 g/L and 24 g/L. In some embodiments, the associated threshold value is between or between about 24 g/L and 32 g/L or 24 g/L and 28 g/L. In some embodiments, the associated threshold value is between or between about 28 g/L and 32 g/L. In some embodiments, the associated threshold value is between or between about 4.3 g/L and 32 g/L. In some embodiments, the associated threshold value is between or between about 5.3 g/L and 12 g/L. In some embodiments, the associated threshold value is 9.7 g/L.

In some embodiments, the tumor burden markers include the blood sample B-cell maturation antigen (sBCMA) level. In some embodiments, increased blood sample BCMA level is correlated with unfavorable patient outcome. In some embodiments, decreased blood sample BCMA level is correlated with favorable patient outcome.

In some embodiments, the parameter of the sBCMA of a patient predicted not to exhibit the clinical response is greater than or equal to an associated threshold value. In some embodiments, the parameter of the sBCMA of a patient predicted to exhibit the clinical response is less than or equal to the associated threshold value. In some embodiments, the associated threshold value is between or between about 30 to 1330 ng/mL. In some embodiments, the associated threshold value is between or between about 30 to 1230 ng/mL, 30 to 1130 ng/mL, 30 to 1030 ng/mL, 30 to 930 ng/mL, 30 to 830 ng/mL, 30 to 730 ng/mL, 30 to 630 ng/mL, 30 to 530 ng/mL, 30 to 430 ng/mL, 30 to 330 ng/mL, 30 to 230 ng/mL, or 30 to 130 ng/mL. In some embodiments, the associated threshold value is between or between about 130 to 1330 ng/mL, 130 ng/mL to 1230 ng/mL, 130 to 1130 ng/mL, 130 to 1030 ng/mL, 130 to 930 ng/mL, 130 to 830 ng/mL, 130 to 730 ng/mL, 130 to 630 ng/mL, 130 to 530 ng/mL, 130 to 430 ng/mL, 130 to 330 ng/mL, or 130 to 230 ng/mL. In some embodiments, the associated threshold value is between or between about 230 to 1330 ng/mL, 230 ng/mL to 1230 ng/mL, 230 to 1130 ng/mL, 230 to 1030 ng/mL, 230 to 930 ng/mL, 230 to 830 ng/mL, 230 to 730 ng/mL, 230 to 630 ng/mL, 230 to 530 ng/mL, 230 to 430 ng/mL, or 230 to 330 ng/mL. In some embodiments, the associated threshold value is between or between about 330 to 1330 ng/mL, 330 ng/mL to 1230 ng/mL, 330 to 1130 ng/mL, 330 to 1030 ng/mL, 330 to 930 ng/mL, 330 to 830 ng/mL, 330 to 730 ng/mL, 330 to 630 ng/mL, 330 to 530 ng/mL, or 330 to 430 ng/mL. In some embodiments, the associated threshold value is between or between about 430 to 1330 ng/mL, 430 ng/mL to 1230 ng/mL, 430 to 1130 ng/mL, 430 to 1030 ng/mL, 430 to 930 ng/mL, 430 to 830 ng/mL, 430 to 730 ng/mL, 430 to 630 ng/mL, or 430 to 530 ng/mL. In some embodiments, the associated threshold value is between or between about 530 to 1330 ng/mL, 530 ng/mL to 1230 ng/mL, 530 to 1130 ng/mL, 530 to 1030 ng/mL, 530 to 930 ng/mL, 530 to 830 ng/mL, 530 to 730 ng/mL, or 530 to 630 ng/mL. In some embodiments, the associated threshold value is between or between about 630 to 1330 ng/mL, 630 ng/mL to 1230 ng/mL, 630 to 1130 ng/mL, 630 to 1030 ng/mL, 630 to 930 ng/mL, 630 to 830 ng/mL or 630 to 730 ng/mL. In some embodiments, the associated threshold value is between or between about 730 to 1330 ng/mL, 730 ng/mL to 1230 ng/mL, 730 to 1130 ng/mL, 730 to 1030 ng/mL, 730 to 930 ng/mL, or 730 to 830 ng/mL. In some embodiments, the associated threshold value is between or between about 830 to 1330 ng/mL, 830 ng/mL to 1230 ng/mL, 830 to 1130 ng/mL, 830 to 1030 ng/mL, or 830 to 930 ng/mL. In some embodiments, the associated threshold value is between or between about 930 to 1330 ng/mL, 930 ng/mL to 1230 ng/mL, 930 to 1130 ng/mL, or 930 to 1030 ng/mL. In some embodiments, the associated threshold value is between or between about 1030 to 1330 ng/mL, 1030 ng/mL to 1230 ng/mL, or 1030 to 1130 ng/mL. In some embodiments, the associated threshold value is between or between about 1130 to 1330 ng/mL or 1130 ng/mL to 1230 ng/mL. In some embodiments, the associated threshold value is between or between about 1230 ng/mL to 1330 ng/mL. In some embodiments, the associated threshold value is between or between about 35 ng/mL to 1300 ng/mL. In some embodiments, the associated threshold values is between or between about 170 ng/mL to 654 ng/mL. In some embodiments, the associated threshold value is or is about 315 ng/mL.

### 6. Immune Profile Markers

Various markers associated with the immune profile (e.g., immune health) of the subject are contemplated for use according to the methods provided herein, e.g., methods of using thresholds of one or a combination of the markers or methods of machine learning. A subject to be treated with a T cell therapy may also be referred to herein as a patient.

In some embodiments, the immune profile markers include the blood sample absolute lymphocyte count (ALC). In some embodiments, decreased blood sample ALC is correlated with unfavorable patient outcome. In some embodiments, increased blood sample ALC is correlated with favorable patient outcome.

In some embodiments, the parameter of the blood sample ALC of a patient predicted not to exhibit the clinical response is greater than or equal to than an associated threshold value. In some embodiments, the parameter of the blood sample ALC of a patient predicted to exhibit the clinical response is less than or equal to the associated threshold value. In some embodiments, the associated threshold value is between or between about 0.3×10⁹ ALC/L and 1.0×10⁹ ALC/L. In some embodiments, the associated threshold value is between or between about 0.3×10⁹ ALC/L and 0.9×10⁹ ALC/L, 0.3×10⁹ ALC/L and 0.8×10⁹ ALC/L, 0.3×10⁹ ALC/L and 0.7×10⁹ ALC/L, 0.3×10⁹ ALC/L and 0.6×10⁹ ALC/L, 0.3×10⁹ ALC/L and 0.5×10⁹ ALC/L, or 0.3×10⁹ ALC/L and 0.4×10⁹ ALC/L. In some embodiments, the associated threshold value is between or between about 0.4×10⁹ ALC/L and 1.0×10⁹ ALC/L, 0.4×10⁹ ALC/L and 0.9×10⁹ ALC/L, 0.4×10⁹ ALC/L and 0.8×10⁹ ALC/L, 0.4×10⁹ ALC/L and 0.7×10⁹ ALC/L, 0.4×10⁹ ALC/L and 0.6×10⁹ ALC/L, or 0.4×10⁹ ALC/L and 0.5×10⁹ ALC/L. In some embodiments, the associated threshold value is between or between about 0.5×10⁹ ALC/L and 1.0×10⁹ ALC/L, 0.5×10⁹ ALC/L and 0.9×10⁹ ALC/L, 0.5×10⁹ ALC/L and 0.8×10⁹ ALC/L, 0.5×10⁹ ALC/L and 0.7×10⁹ ALC/L, or 0.5×10⁹ ALC/L and 0.6×10⁹ ALC/L. In some embodiments, the associated threshold value is between or between about 0.6×10⁹ ALC/L and 1.0×10⁹ ALC/L, 0.6×10⁹ ALC/L and 0.9×10⁹ ALC/L, 0.6×10⁹ ALC/L and 0.8×10⁹ ALC/L, or 0.6×10⁹ ALC/L and 0.7×10⁹ ALC/L. In some embodiments, the associated threshold value is between or between about 0.7×10⁹ ALC/L and 1.0×10⁹ ALC/L, 0.7×10⁹ ALC/L and 0.9×10⁹ ALC/L, or 0.7×10⁹ ALC/L and 0.8×10⁹ ALC/L. In some embodiments, the associated threshold value is between or between about 0.8×10⁹ ALC/L and 1.0×10⁹ ALC/L or 0.8×10⁹ ALC/L and 0.9×10⁹ ALC/L. In some embodiments, the associated threshold value is between or between about 0.9×10⁹ ALC/L and 1.0×10⁹ ALC/L. In some embodiments, the associated threshold value is between or between about 0.4×10⁹ ALC/L and 0.7×10⁹ ALC/L. In some embodiments, the associated threshold value is 0.6×10⁹ ALC/L.

In some embodiments, the immune profile markers include the blood sample red blood cell (RBC) count. In some embodiments, the parameter of the blood sample RBC count of a patient predicted not to exhibit the clinical response is greater than or equal to an associated threshold value. In some embodiments, the parameter of the blood sample RBC count of a patient predicted to exhibit the clinical response is less than or equal to the associated threshold value. In some embodiments, the associated threshold value is between or between about 2.4×10¹² RBC/L and 3.8×10¹² RBC/L. In some embodiments, the associated threshold value is between or between about 2.4×10¹² RBC/L and 3.6×10¹² RBC/L, 2.4×10¹² RBC/L and 3.4×10¹² RBC/L, 2.4×10¹² RBC/L and 3.2×10¹² RBC/L, 2.4×10¹² RBC/L and 3.0×10¹² RBC/L, 2.4×10¹² RBC/L and 2.8×10¹² RBC/L, or 2.4×10¹² RBC/L and 2.6×10¹² RBC/L. In some embodiments, the associated threshold value is between or between about 2.6×10¹² RBC/L and 3.8×10¹² RBC/L, 2.6×10¹² RBC/L and 3.6×10¹² RBC/L, 2.6×10¹² RBC/L and 3.4×10¹² RBC/L, 2.6×10¹² RBC/L and 3.2×10¹² RBC/L, 2.6×10¹² RBC/L and 3.0×10¹² RBC/L, or 2.6×10¹² RBC/L and 2.8×10¹² RBC/L. In some embodiments, the associated threshold value is between or between about 2.8×10¹² RBC/L and 3.8×10¹² RBC/L, 2.8×10¹² RBC/L and 3.6×10¹² RBC/L, 2.8×10¹² RBC/L and 3.4×10¹² RBC/L, 2.8×10¹² RBC/L and 3.2×10¹² RBC/L, or 2.8×10¹² RBC/L and 3.0×10¹² RBC/L. In some embodiments, the associated threshold value is between or between about 3.0×10¹² RBC/L and 3.8×10¹² RBC/L, 3.0×10¹² RBC/L and 3.6×10¹² RBC/L, 3.0×10¹² RBC/L and 3.4×10¹² RBC/L, or 3.0×10¹² RBC/L and 3.2×10¹² RBC/L. In some embodiments, the associated threshold value is between or between about 3.2×10¹² RBC/L and 3.8×10¹² RBC/L, 3.2×10¹² RBC/L and 3.6×10¹² RBC/L, or 3.2×10¹² RBC/L and 3.4×10¹² RBC/L. In some embodiments, the associated threshold value is between or between about 3.4×10¹² RBC/L and 3.8×10¹² RBC/L or 3.4×10¹² RBC/L and 3.6×10¹² RBC/L. In some embodiments, the associated threshold value is between or between about 3.6×10¹² RBC/L and 3.8×10¹² RBC/L. In some embodiments, the associated threshold value is between or between about 3.6×10¹² RBC/L and 3.8×10¹² RBC/L. In some embodiments, the associated threshold value is between or between about 2.4×10¹² RBC/L and 3.7×10¹² RBC/L. In some embodiments, the associated threshold value is between or between about 2.9×10¹² RBC/L and 3.3×10¹² RBC/L. In some embodiments, the associated threshold value is or is about 3.0×10¹² RBC/L.

In some embodiments, the immune profile markers include the blood sample white blood cell (WBC) count. In some embodiments, decreased blood sample WBC count is correlated with unfavorable patient outcome. In some embodiments, increased blood sample WBC is correlated with favorable patient outcome.

In some embodiments, the parameter of the blood sample WBC count of a patient predicted not to exhibit the clinical response is greater than or equal than an associated threshold value. In some embodiments, the parameter of the blood sample WBC count of a patient predicted to exhibit the clinical response is less than or equal to the associated threshold value. In some embodiments, the associated threshold value is between or between about 2.1×10⁹ WBC/L and 7.1×10⁹ WBC/L. In some embodiments, the associated threshold value is between or between about 2.1×10⁹ WBC/L and 6.6×10⁹ WBC/L, 2.1×10⁹ WBC/L and 6.1×10⁹ WBC/L, 2.1×10⁹ WBC/L and 5.6×10⁹ WBC/L, 2.1×10⁹ WBC/L and 5.1×10⁹ WBC/L, 2.1×10⁹ WBC/L and 4.6×10⁹ WBC/L, 2.1×10⁹ WBC/L and 4.1×10⁹ WBC/L, 2.1×10⁹ WBC/L and 3.6×10⁹ WBC/L, 2.1×10⁹ WBC/L and 3.1×10⁹ WBC/L, or 2.1×10⁹ WBC/L and 2.6×10⁹ WBC/L. In some embodiments, the associated threshold value is between or between about 2.6×10⁹ WBC/L and 7.1×10⁹ WBC/L, 2.6×10⁹ WBC/L and 6.6×10⁹ WBC/L, 2.6×10⁹ WBC/L and 6.1×10⁹ WBC/L, 2.6×10⁹ WBC/L and 5.6×10⁹ WBC/L, 2.6×10⁹ WBC/L and 5.1×10⁹ WBC/L, 2.6×10⁹ WBC/L and 4.6×10⁹ WBC/L, 2.6×10⁹ WBC/L and 4.1×10⁹ WBC/L, 2.6×10⁹ WBC/L and 3.6×10⁹ WBC/L, or 2.6×10⁹ WBC/L and 3.1×10⁹ WBC/L. In some embodiments, the associated threshold value is between or between about 3.1×10⁹ WBC/L and 7.1×10⁹ WBC/L, 3.1×10⁹ WBC/L and 6.6×10⁹ WBC/L, 3.1×10⁹ WBC/L and 6.1×10⁹ WBC/L, 3.1×10⁹ WBC/L and 5.6×10⁹ WBC/L, 3.1×10⁹ WBC/L and 5.1×10⁹ WBC/L, 3.1×10⁹ WBC/L and 4.6×10⁹ WBC/L, 3.1×10⁹ WBC/L and 4.1×10⁹ WBC/L, or 3.1×10⁹ WBC/L and 3.6×10⁹ WBC/L. In some embodiments, the associated threshold value is between or between about 3.6×10⁹ WBC/L and 7.1×10⁹ WBC/L, 3.6×10⁹ WBC/L and 6.6×10⁹ WBC/L, 3.6×10⁹ WBC/L and 6.1×10⁹ WBC/L, 3.6×10⁹ WBC/L and 5.6×10⁹ WBC/L, 3.6×10⁹ WBC/L and 5.1×10⁹ WBC/L, 3.6×10⁹ WBC/L and 4.6×10⁹ WBC/L, or 3.6×10⁹ WBC/L and 4.1×10⁹ WBC/L. In some embodiments, the associated threshold value is between or between about 4.1×10⁹ WBC/L and 7.1×10⁹ WBC/L, 4.1×10⁹ WBC/L and 6.6×10⁹ WBC/L, 4.1×10⁹ WBC/L and 6.1×10⁹ WBC/L, 4.1×10⁹ WBC/L and 5.6×10⁹ WBC/L, 4.1×10⁹ WBC/L and 5.1×10⁹ WBC/L, or 4.1×10⁹ WBC/L and 4.6×10⁹ WBC/L. In some embodiments, the associated threshold value is between or between about 4.6×10⁹ WBC/L and 7.1×10⁹ WBC/L, 4.6×10⁹ WBC/L and 6.6×10⁹ WBC/L, 4.6×10⁹ WBC/L and 6.1×10⁹ WBC/L, 4.6×10⁹ WBC/L and 5.6×10⁹ WBC/L, or 4.6×10⁹ WBC/L and 5.1×10⁹ WBC/L. In some embodiments, the associated threshold value is between or between about 5.1×10⁹ WBC/L and 7.1×10⁹ WBC/L, 5.1×10⁹ WBC/L and 6.6×10⁹ WBC/L, 5.1×10⁹ WBC/L and 6.1×10⁹ WBC/L, or 5.1×10⁹ WBC/L and 5.6×10⁹ WBC/L. In some embodiments, the associated threshold value is between or between about 5.6×10⁹ WBC/L and 7.1×10⁹ WBC/L, 5.6×10⁹ WBC/L and 6.6×10⁹ WBC/L, or 5.6×10⁹ WBC/L and 6.1×10⁹ WBC/L. In some embodiments, the associated threshold value is between or between about 6.1×10⁹ WBC/L and 7.1×10⁹ WBC/L or 6.1×10⁹ WBC/L and 6.6×10⁹ WBC/L. In some embodiments, the associated threshold value is between or between about 6.6×10⁹ WBC/L and 7.1×10⁹ WBC/L. In some embodiments, the associated threshold value is between or between about 2.9×10⁹ WBC/L and 4.2×10⁹ WBC/L. In some embodiments, the associated threshold value is or is about 3.5×10⁹ WBC/L.

In some embodiments, the immune profile markers include the d-dimer concentration. In some embodiments, the parameter of the d-dimer concentration a patient predicted not to exhibit the clinical response is greater than or equal to an associated threshold value. In some embodiments, the parameter of the d-dimer concentration of a patient predicted to exhibit the clinical response is less than or equal to the associated threshold value. In some embodiments, the associated threshold value is between or between about 0.5 mg/L and 11 mg/L. In some embodiments, the associated threshold value is between or between about 0.5 mg/L and 9 mg/L, 0.5 mg/L and 7 mg/L, 0.5 mg/L and 5 mg/L, 0.5 mg/L and 3 mg/L, or 0.5 mg/L and 1 mg/L. In some embodiments, the associated threshold value is between or between about 1.5 mg/L and 11 mg/L, 1.5 mg/L and 9 mg/L, 1.5 mg/L and 7 mg/L, 1.5 mg/L and 5 mg/L, or 1.5 mg/L and 3 mg/L. In some embodiments, the associated threshold value is between or between about 3.5 mg/L and 11 mg/L, 3.5 mg/L and 9 mg/L, 3.5 mg/L and 7 mg/L, or 3.5 mg/L and 5 mg/L. In some embodiments, the associated threshold value is between or between about 5.5 mg/L and 11 mg/L, 5.5 mg/L and 9 mg/L, or 5.5 mg/L and 7 mg/L. In some embodiments, the associated threshold value is between or between about 7.5 mg/L and 11 mg/L or 7.5 mg/L and 9 mg/L. In some embodiments, the associated threshold value is between or between about 9.5 mg/L and 11 mg/L. In some embodiments, the associated threshold value is between or between about 0.5 mg/L and 1.3 mg/L. In some embodiments, the associated threshold value is or is about 0.9 mg/L.

In some embodiments, the immune profile markers include the fibrinogen concentration. In some embodiments, the parameter of the fibrinogen concentration a patient predicted not to exhibit the clinical response is greater than or equal to an associated threshold value. In some embodiments, the parameter of the fibrinogen concentration of a patient predicted to exhibit the clinical response is less than or equal to the associated threshold value. In some embodiments, the associated threshold value is between or between about 2.0 g/L and 8.0 g/L. In some embodiments, the associated threshold value is between or between about 2.0 g/L and 7.0 g/L, 2.0 g/L and 6.0 g/L, 2.0 g/L and 5.0 g/L, 2.0 g/L and 4.0 g/L, or 2.0 g/L and 3.0 g/L. In some embodiments, the associated threshold value is between or between about 3.0 g/L and 8.0 g/L, 3.0 g/L and 7.0 g/L, 3.0 g/L and 6.0 g/L, 3.0 g/L and 5.0 g/L, or 3.0 g/L and 4.0 g/L. In some embodiments, the associated threshold value is between or between about 4.0 g/L and 8.0 g/L, 4.0 g/L and 7.0 g/L, 4.0 g/L and 6.0 g/L, or 4.0 g/L and 5.0 g/L. In some embodiments, the associated threshold value is between or between about 5.0 g/L and 8.0 g/L, 5.0 g/L and 7.0 g/L or 5.0 g/L and 6.0 g/L. In some embodiments, the associated threshold value is between or between about 6.0 g/L and 8.0 g/L or 6.0 g/L and 7.0 g/L. In some embodiments, the associated threshold value is between or between about 2.2 g/L and 7.7 g/L. In some embodiments, the associated threshold value is between or between about 4.2 g/L and 5.4 g/L. In some embodiments, the associated threshold value is 5.1 g/L.

In some embodiments, the immune profile markers include the absolute monocyte count. In some embodiments, the parameter of the absolute monocyte count of a patient predicted not to exhibit the clinical response is greater than or equal to the associated threshold value. In some embodiments, the parameter of the absolute monocyte count of a patient predicted to exhibit the clinical response is less than or equal to the associated threshold value. In some embodiments, the associated threshold value is between or between about 0.2×10⁹ monocytes/L and 1.2×10⁹ monocytes/L. In some embodiments, the associated threshold value is between or between about 0.2×10⁹ monocytes/L and 1.0×10⁹ monocytes/L, 0.2×10⁹ monocytes/L and 0.8×10⁹ monocytes/L, 0.2×10⁹ monocytes/L and 0.6×10⁹ monocytes/L, or 0.2×10⁹ monocytes/L and 0.4×10⁹ monocytes/L. In some embodiments, the associated threshold value is between or between about 0.4×10⁹ monocytes/L and 1.2×10⁹ monocytes/L, 0.4×10⁹ monocytes/L and 1.0×10⁹ monocytes/L, 0.4×10⁹ monocytes/L and 0.8×10⁹ monocytes/L, or 0.4×10⁹ monocytes/L and 0.6×10⁹ monocytes/L. In some embodiments, the associated threshold value is between or between about 0.6×10⁹ monocytes/L and 1.2×10⁹ monocytes/L, 0.6×10⁹ monocytes/L and 1.0×10⁹ monocytes/L, or 0.6×10⁹ monocytes/L and 0.8×10⁹ monocytes/L. In some embodiments, the associated threshold value is between or between about 0.8×10⁹ monocytes/L and 1.2×10⁹ monocytes/L or 0.8×10⁹ monocytes/L and 1.0×10⁹ monocytes/L. In some embodiments, the associated threshold value is between or between about 1.0×10⁹ monocytes/L and 1.2×10⁹ monocytes/L. In some embodiments, the associated threshold value is between or between about 0.2×10⁹ monocytes/L and 1.1×10⁹ monocytes/L. In some embodiments, the associated threshold value is between or between about 0.4×10⁹ monocytes/L and 0.7×10⁹ monocytes/L. In some embodiments, the associated threshold value is or is about 0.6×10⁹ monocytes/L.

In some embodiments, the immune profile markers include the monocyte to leukocyte ratio. In some embodiments, increased monocyte to leukocyte ratio is correlated with unfavorable patient outcome. In some embodiments, decreased monocyte to leukocyte ratio is correlated with favorable patient outcome.

In some embodiments, the parameter of the monocyte to leukocyte ratio of a patient predicted not to exhibit the clinical response is greater than or equal to an associated threshold value. In some embodiments, the parameter of the monocyte to leukocyte ratio of a patient predicted to exhibit the clinical response is less than or equal to the associated threshold value. In some embodiments, the associated threshold value is between or between about 6 and 18. In some embodiments, the associated threshold value is between or between about 6 and 16, 6 and 14, 6 and 12, 6 and 10, or 6 and 8. In some embodiments, the associated threshold value is between or between about 8 and 18, 8 and 16, 8 and 14, 8 and 12, or 8 and 10. In some embodiments, the associated threshold value is between or between about 10 and 18, 10 and 16, 10 and 14, or 10 and 12. In some embodiments, the associated threshold value is between or between about 12 and 18, 12 and 16, or 12 and 14. In some embodiments, the associated threshold value is between or between about 14 and 18 or 14 and 16. In some embodiments, the associated threshold value is between or between about 16 and 18. In some embodiments, the associated threshold value is between or between about 6.7 and 18. In some embodiments, the associated threshold value is between or between about 13 and 14. In some embodiments, the associated threshold value is or is about 13.

### 7. Fitness Markers

Various markers associated with the fitness of the subject are contemplated for use according to the methods provided herein, e.g., methods of using thresholds of one or a combination of the markers or methods of machine learning. A subject to be treated with a T cell therapy may also be referred to herein as a patient.

In some embodiments, the patient fitness markers include age. In some embodiments, the parameter of the age of a patient predicted not to exhibit the clinical response is greater than associated threshold value. In some embodiments, the parameter of the age of a patient predicted to exhibit the clinical response is less than the associated threshold value. In some embodiments, the associated threshold value is between or between about 56 years and 66 years. In some embodiments, the associated threshold value is between or between about 56 years and 64 years, 56 years and 62 years, or 56 years and 60 years. In some embodiments, the associated threshold value is between or between about 58 years and 66 years, 58 years 64 years, 58 years and 62 years, or 58 years and 60 years. In some embodiments, the associated threshold value is between or between about 60 years and 66 years, 60 years and 64 years, or 60 years and 62 years. In some embodiments, the associated threshold value is between or between about 62 years and 66 years or 62 years and 64 years. In some embodiments, the associated threshold value is between or between about 64 years and 66 years. In some embodiments, the associated threshold value is between or between about 57 years and 66 years. In some embodiments, the associated threshold value is 65 years.

In some embodiments, the patient fitness markers include BMI. In some embodiments, the parameter of the BMI of a patient predicted not to exhibit the clinical response is less than the associated threshold value. In some embodiments, the parameter of the BMI of a patient predicted to exhibit the clinical response is greater than the associated threshold value. In some embodiments, the associated threshold value is between or between about 22 and 32. In some embodiments, the associated threshold value is between or between about 22 and 30, 22 and 28, 22 and 26, or 22 and 24. In some embodiments, the associated threshold value is between or between about 24 and 32, 24 and 30, 24 and 28, or 24 and 26. In some embodiments, the associated threshold value is between or between about 26 and 32, 26 and 30, or 26 and 28. In some embodiments, the associated threshold value is between or between about 28 and 32 or 28 and 30. In some embodiments, the associated threshold value is between or between about 30 and 32. In some embodiments, the associated threshold value is between or between about 22 and 31. In some embodiments, the associated threshold value is between or between about 23 and 29. In some embodiments, the associated threshold value is 26.

In some embodiments, the patient fitness markers include blood sample albumin level. In some embodiments, decreased blood sample albumin level is correlated with unfavorable patient outcome. In some embodiments, increased blood sample albumin level is correlated with favorable patient outcome.

In some embodiments, the parameter of the blood sample albumin level of a patient predicted not to exhibit the clinical response is less than the associated threshold value. In some embodiments, the parameter of the blood sample albumin level of a patient predicted to exhibit the clinical response is greater than the associated threshold value. In some embodiments, the associated threshold value is between or between about 31 g/L and 41 g/L. In some embodiments, the associated threshold value is between or between about 31 g/L and 39 g/L, 31 g/L and 37 g/L, 31 g/L and 35 g/L, or 31 g/L and 33 g/L. In some embodiments, the associated threshold value is between or between about 33 g/L and 41 g/L, 33 g/L and 39 g/L, 33 g/L and 37 g/L, or 33 g/L and 35 g/L. In some embodiments, the associated threshold value is between or between about 35 g/L and 41 g/L, 35 g/L and 39 g/L, or 35 g/L and 37 g/L. In some embodiments, the associated threshold value is between or between about 37 g/L and 41 g/L or 37 g/L and 39 g/L. In some embodiments, the associated threshold value is between or between about 39 g/L and 41 g/L. In some embodiments, the associated threshold value is between or between about 36 g/L and 40 g/L.

In some embodiments, the patient fitness markers include the alkaline phosphatase concentration. In some embodiments, the parameter of the alkaline phosphatase concentration of a patient predicted not to exhibit the clinical response is less than the associated threshold value. In some embodiments, the parameter of the alkaline phosphatase concentration of a patient predicted to exhibit the clinical response is greater than the associated threshold value. In some embodiments, the associated threshold value is between or between about 28 U/L and 138 U/L. In some embodiments, the associated threshold value is between or between about 28 U/L and 118 U/L, 28 U/L and 98 U/L, 28 U/L and 78 U/L, 28 U/L and 58 U/L, or 28 U/L and 38 U/L. In some embodiments, the associated threshold value is between or between about 48 U/L and 118 U/L, 48 U/L and 98 U/L, 48 U/L and 78 U/L, or 48 U/L and 58 U/L. In some embodiments, the associated threshold value is between or between about 68 U/L and 118 U/L, 68 U/L and 98 U/L, or 68 U/L and 78 U/L. In some embodiments, the associated threshold value is between or between about 88 U/L and 118 U/L or 88 U/L and 98 U/L. In some embodiments, the associated threshold value is between or between about 108 U/L and 118 U/L. In some embodiments, the associated threshold value is between or between about 28 U/L and 134 U/L. In some embodiments, the associated threshold value is between or between about 54 U/L and 64 U/L. In some embodiments, the associated threshold value is 58 U/L.

In some embodiments, the patient fitness markers include the aspartate aminotransferase concentration. In some embodiments, the parameter of the aspartate aminotransferase concentration of a patient predicted not to exhibit the clinical response is less than the associated threshold value. In some embodiments, the parameter of the aspartate aminotransferase concentration of a patient predicted to exhibit the clinical response is greater than the associated threshold value. In some embodiments, the associated threshold value is between or between about 7 U/L and 49 U/L. In some embodiments, the associated threshold value is between or between about 7 U/L and 42 U/L, 7 U/L and 35 U/L, 7 U/L and 28 U/L, 7 U/L and 21 U/L, or 7 U/L and 14 U/L. In some embodiments, the associated threshold value is between or between about 14 U/L to 49 U/L, 14 U/L and 42 U/L, 14 U/L and 35 U/L, 14 U/L and 28 U/L, or 14 U/L and 21 U/L. In some embodiments, the associated threshold value is between or between about 21 U/L to 49 U/L, 21 U/L and 42 U/L, 21 U/L and 35 U/L, or 21 U/L and 28 U/L. In some embodiments, the associated threshold value is between or between about 28 U/L to 49 U/L, 28 U/L and 42 U/L, or 28 U/L and 35 U/L. In some embodiments, the associated threshold value is between or between about 35 U/L to 49 U/L or 35 U/L and 42 U/L. In some embodiments, the associated threshold value is between or between about 42 U/L and 49 U/L. In some embodiments, the associated threshold value is between or between about 7.3 U/L and 49 U/L. In some embodiments, the associated threshold value is between or between about 16 U/L and 26 U/L. In some embodiments, the associated threshold value is 21 U/L.

In some embodiments, the patient fitness markers include the alanine aminotransferase concentration. In some embodiments, the parameter of the alanine aminotransferase concentration of a patient predicted not to exhibit the clinical response is less than the associated threshold value. In some embodiments, the parameter of the alanine aminotransferase concentration of a patient predicted to exhibit the clinical response is greater than the associated threshold value. In some embodiments, the associated threshold value is between or between about 8 U/L and 32 U/L. In some embodiments, the associated threshold value is between or between about 8 U/L and 28 U/L, 8 U/L and 24 U/L, 8 U/L and 20 U/L, 8 U/L and 16 U/L, or 8 U/L and 12 U/L. In some embodiments, the associated threshold value is between or between about 12 U/L and 32 U/L, 12 U/L and 28 U/L, 12 U/L and 24 U/L, 12 U/L and 20 U/L, or 12 U/L and 16 U/L. In some embodiments, the associated threshold value is between or between about 16 U/L and 32 U/L, 16 U/L and 28 U/L, 16 U/L and 24 U/L, or 16 U/L and 20 U/L. In some embodiments, the associated threshold value is between or between about 20 U/L and 32 U/L, 20 U/L and 28 U/L, or 20 U/L and 24 U/L. In some embodiments, the associated threshold value is between or between about 24 U/L and 32 U/L or 24 U/L and 28 U/L. In some embodiments, the associated threshold value is between or between about 28 U/L and 32 U/L. In some embodiments, the associated threshold value is between or between about 8 U/L and 31 U/L. In some embodiments, the associated threshold value is between or between about 13 U/L and 29 U/L. In some embodiments, the associated threshold value is or is about 18 U/L.

In some embodiments, the patient fitness markers include the creatinine concentration. In some embodiments, the parameter of the creatinine concentration of a patient predicted not to exhibit the clinical response is less than the associated threshold value. In some embodiments, the parameter of the creatinine concentration of a patient predicted to exhibit the clinical response is greater than the associated threshold value. In some embodiments, the associated threshold value is between or between about 40 μmol/L and 120 μmol/L. In some embodiments, the associated threshold value is between or between about 40 μmol/L and 100 μmol/L, 40 μmol/L and 80 μmol/L, or 40 μmol/L and 60 μmol/L. In some embodiments, the associated threshold value is between or between about 60 μmol/L and 120 μmol/L, 60 μmol/L and 100 μmol/L, or 60 μmol/L and 80 μmol/L. In some embodiments, the associated threshold value is between or between about 80 μmol/L and 120 μmol/L or 80 μmol/L and 100 μmol/L. In some embodiments, the associated threshold value is between or between about 100 μmol/L and 120 μmol/L. In some embodiments, the associated threshold value is between or between about 46 μmol/L and 114 μmol/L. In some embodiments, the associated threshold value is between or between about 52 μmol/L and 80 μmol/L. In some embodiments, the associated threshold value is 60 μmol/L.

In some embodiments, the patient fitness markers include the creatinine clearance. In some embodiments, the parameter of the creatinine clearance of a patient predicted not to exhibit the clinical response is less than the associated threshold value. In some embodiments, the parameter of the creatinine clearance of a patient predicted to exhibit the clinical response is greater than the associated threshold value. In some embodiments, the associated threshold value is between or between about 0.8 mL/s and 2.0 mL/s. In some embodiments, the associated threshold value is between or between about 0.8 mL/s and 1.8 mL/s, 0.8 mL/s and 1.6 mL/s, 0.8 mL/s and 1.4 mL/s, 0.8 mL/s and 1.2 mL/s, or 0.8 mL/s and 1.0 mL/s. In some embodiments, the associated threshold value is between or between about 1.0 mL/s and 2.0 mL/s, 1.0 mL/s and 1.8 mL/s, 1.0 mL/s and 1.6 mL/s, 1.0 mL/s and 1.4 mL/s, or 1.0 mL/s and 1.2 mL/s. In some embodiments, the associated threshold value is between or between about 1.2 mL/s and 2.0 mL/s, 1.2 mL/s and 1.8 mL/s, 1.2 mL/s and 1.6 mL/s, or 1.2 mL/s and 1.4 mL/s. In some embodiments, the associated threshold value is between or between about 1.4 mL/s and 2.0 mL/s, 1.4 mL/s and 1.8 mL/s, or 1.4 mL/s and 1.6 mL/s. In some embodiments, the associated threshold value is between or between about 1.6 mL/s and 2.0 mL/s or 1.6 mL/s and 1.8 mL/s. In some embodiments, the associated threshold value is between or between about 1.8 mL/s and 2.0 mL/s. In some embodiments, the associated threshold value is between or between about 1.9 mL/s and 2.0 mL/s. In some embodiments, the associated threshold value is or is about 2.0 mL/s.

In some embodiments, the patient fitness markers include the direct bilirubin concentration. In some embodiments, the parameter of the direct bilirubin concentration of a patient predicted not to exhibit the clinical response is greater than or equal to the associated threshold value. In some embodiments, the parameter of the direct bilirubin concentration of a patient predicted to exhibit the clinical response is less than or equal to than the associated threshold value. In some embodiments, the associated threshold value is between or between about 1.4 μmol/L and 2.8 μmol/L. In some embodiments, the associated threshold value is between or between about 1.4 μmol/L and 2.4 μmol/L, 1.4 μmol/L and 2.0 μmol/L, or 1.4 μmol/L and 1.6 μmol/L. In some embodiments, the associated threshold value is between or between about 2.0 μmol/L and 2.8 μmol/L or 2.0 μmol/L and 2.4 μmol/L. In some embodiments, the associated threshold value is between or between about 2.4 μmol/L and 2.8 μmol/L. In some embodiments, the associated threshold value is between or between about 1.4 μmol/L and 2.7 μmol/L. In some embodiments, the associated threshold value is between or between about 1.8 μmol/L and 2.2 μmol/L. In some embodiments, the associated threshold value is or is about 2.2 μmol/L.

In some embodiments, the patient fitness markers include the bilirubin concentration. In some embodiments, the parameter of the bilirubin concentration of a patient predicted not to exhibit the clinical response is greater than or equal to the associated threshold value. In some embodiments, the parameter of the bilirubin concentration of a patient predicted to exhibit the clinical response is less than or equal to than the associated threshold value. In some embodiments, the associated threshold value is between or between about 3 μmol/L and 24 μmol/L. In some embodiments, the associated threshold value is between or between about 3 μmol/L and 24 μmol/L, 3 μmol/L and 21 μmol/L, 3 μmol/L and 18 μmol/L, 3 μmol/L and 15 μmol/L, 3 μmol/L and 12 μmol/L, 3 μmol/L and 9 μmol/L, or 3 μmol/L and 6 μmol/L. In some embodiments, the associated threshold value is between or between about 6 μmol/L and 24 μmol/L, 6 μmol/L and 21 μmol/L, 6 μmol/L and 18 μmol/L, 6 μmol/L and 15 μmol/L, 6 μmol/L and 12 μmol/L, or 6 μmol/L and 9 μmol/L. In some embodiments, the associated threshold value is between or between about 9 μmol/L and 24 μmol/L, 9 μmol/L and 21 μmol/L, 9 μmol/L and 18 μmol/L, 9 μmol/L and 15 μmol/L, or 9 μmol/L and 12 μmol/L. In some embodiments, the associated threshold value is between or between about 12 μmol/L and 24 μmol/L, 12 μmol/L and 21 μmol/L, 12 μmol/L and 18 μmol/L, or 12 μmol/L and 15 μmol/L. In some embodiments, the associated threshold value is between or between about 15 μmol/L and 24 μmol/L, 15 μmol/L and 21 μmol/L, or 15 μmol/L and 18 μmol/L. In some embodiments, the associated threshold value is between or between about 18 μmol/L and 24 μmol/L or 18 μmol/L and 21 μmol/L. In some embodiments, the associated threshold value is between or between about 21 μmol/L and 24 μmol/L. In some embodiments, the associated threshold value is between or between about 3.4 μmol/L and 23 μmol/L. In some embodiments, the associated threshold value is between or between about 9.4 μmol/L and 9.6 μmol/L. In some embodiments, the associated threshold value is or is about 9.4 μmol/L.

### B. Machine Learning Methods

In some embodiments, the provided methods involve providing a parameter of a marker or parameters of a combination of markers of a subject to a process that includes a machine learning model. In some embodiments, the machine learning model is trained to predict, based on the marker or combination of markers, whether or not a subject will exhibit a clinical response to a T cell therapy. In some embodiments, the machine learning model is trained to predict, based on the marker or combination of markers, if a therapeutically effective T cell therapy can or cannot be manufactured for the subject. In some embodiments, the provided methods involve prediction using one or more outputs of the process, which can be one or more outputs of or derived from outputs of the machine learning model.

In some embodiments, the provided methods involve providing parameters of a combination of markers of a subject to a process that includes a machine learning model. In some embodiments, the machine learning model is trained to predict, based on the combination of markers, whether or not a subject will exhibit a clinical response to a T cell therapy. In some embodiments, the machine learning model is trained to predict, based on the combination of markers, if a therapeutically effective T cell therapy can or cannot be manufactured for the subject. In some embodiments, the provided methods involve prediction using one or more outputs of the process, which can be one or more outputs of or derived from outputs of the machine learning model. As an example of process outputs derived from outputs of the machine learning, in some embodiments, outputs of the machine learning model are normalized or transformed before being evaluated.

Machine learning models suitable for use in the provided methods can be identified and selected by one of ordinary skill in the art. Methods for training the machine learning model can also be identified and selected by one of ordinary skill in the art. Exemplary methods for training the machine learning model are described in Hastie et al., The Elements of Statistical Learning (2016); and Abu-Mostafa et al., Learning from Data (2012). Exemplary machine learning models are also described in Hastie et al., The Elements of Statistical Learning (2016); and Abu-Mostafa et al., Learning from Data (2012).

In some embodiments, the machine learning model is a non-supervised machine learning model. In some embodiments, the machine learning model is a supervised machine learning model. In some embodiments, the machine learning model is a non-linear model. In some embodiments, the machine learning model is a random forests model. In some embodiments, the machine learning model is a linear model. In some embodiments, the machine learning model is a logistic regression model. In some embodiments, the machine learning model is a linear regression model. In some embodiments, the machine learning model is a polynomial regression model. In some embodiments, the machine learning model is a generalized linear model. In some embodiments, the machine learning model is a binomial regression model.

In some embodiments, the machine learning model is a linear discriminant analysis model. In some embodiments, the machine learning model is a Naive Bayes classifier. In some embodiments, the machine learning model is a perceptron. In some embodiments, the machine learning model is a support vector machine. In some embodiments, the machine learning model is a neural network.

In some embodiments, the machine learning model is a classification model. In some embodiments, the machine learning model is a binary classification model. In some embodiments, the machine learning model is a multiclass classification model.

In some embodiments, the machine learning model is trained using parameters of the marker or combination of markers from a plurality of subjects. In some embodiments, the plurality of subjects have each been administered a T cell therapy. In some embodiments, the T cell therapy is any as described herein. In some embodiments, the machine learning model is trained using clinical responses of the plurality of subjects.

In some embodiments, the plurality of subjects include about 500, 400, 300, 200, 150, 100, 50, 25, 15, or 10 subjects. In some embodiments, the plurality of subjects include about 100 to 500, 100 to 400, 100 to 300, 100 to 200, or 100 to 150 subjects. In some embodiments, the plurality of subjects are subjects participating in a clinical trial.

In some embodiments, the model is trained or evaluated using bootstrap aggregation. In some embodiments, the model is trained or evaluated using cross validation. In some embodiments, the model is trained or evaluated using k-fold cross validation. In some embodiments, the model is trained or evaluated using nested cross validation.

The models described herein may also be used to determine clinical responses of a subject to treatment with a T cell therapy prior to treating the subject with the T cell therapy. In some embodiments, assessing the determined clinical responses of the subject can be used to inform treatment of the subject. For example, if a subject is determined (e.g., predicted) to have negative clinical response, e.g., toxicity, poor or reduced pharmacokinetics compared to a target response, lack of CR, PR, or DOR, an alteration to a predetermined treatment regimen can be made. On the other hand, if a subject is determined (e.g., predicted) to have positive clinical responses, e.g., CR, PR, DOR, a pharmacokinetic response that reflects or is greater than a target pharmacokinetic response, no or mild toxicity, a predetermined treatment regimen may be administered.

In some embodiments, the trained model is used to determine or predict, prior to treatment, if a subject to be treated with a treatment regimen, e.g., a predetermined treatment regimen that includes a T cell therapy, including a T cell therapy produced from an input composition comprising T cells selected from the subject, will exhibit a CR following treatment with the treatment regimen.

In some embodiments, the trained model is used to determine or predict, prior to treatment, if a subject to be treated with a treatment regimen, e.g., a predetermined treatment regimen that includes a T cell therapy, including a T cell therapy produced from an input composition comprising T cells selected from the subject, will exhibit a PR following treatment with the treatment regimen.

In some embodiments, the trained model is used to determine or predict, prior to treatment, if a subject to be treated with a treatment regimen, e.g., a predetermined treatment regimen that includes a T cell therapy, including a T cell therapy produced from an input composition comprising T cells selected from the subject, will exhibit an OR following treatment with the treatment regimen.

In some embodiments, the trained model is used to determine or predict, prior to treatment, the DOR of a subject to be treated with a treatment regimen, e.g., a predetermined treatment regimen that includes a T cell therapy, including a T cell therapy produced from an input composition comprising T cells selected from the subject, following treatment with the treatment regimen. In some embodiments, the trained model is used to determine or predict, prior to treatment, if a subject to be treated with a treatment regimen, e.g., a predetermined treatment regimen that includes a T cell therapy, including a T cell therapy produced from an input composition comprising T cells selected from the subject, will exhibit a durable response, e.g., a DOR of greater than three months, following treatment with the treatment regimen.

In some embodiments, the trained model is used to determine or predict, prior to treatment, the PFS of a subject to be treated with a treatment regimen, e.g., a predetermined treatment regimen that includes a T cell therapy, including a T cell therapy produced from an input composition comprising T cells selected from the subject, following treatment with the treatment regimen. In some embodiments, the trained model is used to determine or predict, prior to treatment, if a subject to be treated with a treatment regimen, e.g., a predetermined treatment regimen that includes a T cell therapy, including a T cell therapy produced from an input composition comprising T cells selected from the subject, will exhibit a PFS of certain duration, e.g., a PFS of greater than three months, following treatment with the treatment regimen.

In some embodiments, the trained model is used to determine or predict, prior to treatment, the pharmacokinetic response of a subject to be treated with a treatment regimen, e.g., a predetermined treatment regimen that includes a T cell therapy, including a T cell therapy produced from an input composition comprising T cells selected from the subject, following treatment with the treatment regimen. In some embodiments, the trained model is used to determine or predict, prior to treatment, if a subject to be treated with a treatment regimen, e.g., a predetermined treatment regimen that includes a T cell therapy, including a T cell therapy produced from an input composition comprising T cells selected from the subject, will exhibit a pharmacokinetic response greater than a target pharmacokinetic response, following treatment with the treatment regimen. In some embodiments, the pharmacokinetic response is a measure of maximum CAR+ T cell concentration (Cₘₐₓ) in a blood sample obtained from the subject at a period of time after administration of the treatment regimen. In some embodiments, the pharmacokinetic response is a measure of exposure to CAR+ T cells, for instance exposure over or over about 28 days following administration of the treatment regimen and/or as determined by AUC of the CAR+ T cell concentration-time curve following administration of the treatment regimen. In some embodiments, the pharmacokinetic response is the time to peak concentration of CAR+ T cells (Tₘₐₓ).

In some embodiments, the trained model is used to determine or predict, prior to treatment, if a subject to be treated with a treatment regimen, e.g., a predetermined treatment regimen that includes a T cell therapy, including a T cell therapy produced from an input composition comprising T cells selected from the subject, will exhibit a toxicity response following treatment with the treatment regimen. In some embodiments, the toxicity response is CRS. In some embodiments, the toxicity response is severe CRS, e.g., grade 3 or higher CRS. In some embodiments is a neurotoxicity. In some embodiments, the toxicity response is severe neurotoxicity, e.g., grade 3 or higher neurotoxicity.

### II. Methods for Generating a T Cell Therapy

In some embodiments, the provided methods are used in connection with generating a T cell therapy of engineered cells, also referred to herein as an output composition or therapeutic cell composition, such as engineered CD4+ T cells and/or engineered CD8+ T cells, that express a recombinant protein, e.g., a recombinant receptor such as a T cell receptor (TCR) or a chimeric antigen receptor (CAR). In some embodiments, the methods provided herein are used in connection with manufacturing, generating, or producing a cell therapy, and may be used in connection with additional processing steps, such as steps for the isolation, separation, selection, activation or stimulation, transduction, washing, suspension, dilution, concentration, and/or formulation of the cells. In some embodiments, the methods of generating or producing engineered cells, e.g., engineered CD4+ T cells and/or engineered CD8+ T cells, include one or more of isolating cells from a subject, preparing, processing, incubating under stimulating conditions, and/or engineering (e.g., transducing) the cells. In some embodiments, the method includes processing steps carried out in an order in which: input cells, e.g., primary cells, are first isolated, such as selected or separated, from a biological sample; input cells are incubated under stimulating conditions, engineered with vector particles, e.g., viral vector particles, to introduce a recombinant polynucleotide into the cells, e.g., by transduction or transfection; cultivating the engineered cells, e.g., transduced cells, such as to expand the cells; and collecting, harvesting, and/or filling a container with all or a portion of the cells for formulating the cells in an output composition. In some embodiments, CD4+ and CD8+ T cells are manufactured independently from one another, e.g., in separate input compositions, but the process for manufacturing includes the same processing steps. In some embodiments, CD4+ and CD8+ T cells are manufactured together, e.g., in the same input composition.

In some embodiments, the cells of the generated output composition (e.g., therapeutic cell composition) are re-introduced into the same subject, before or after cryopreservation. In some embodiments, the output compositions of engineered cells (e.g., therapeutic cell composition) are suitable for use in a therapy, e.g., an autologous cell therapy. Exemplary manufacturing methods are described in published international patent application, publication no. WO 2019/089855, the contents of which are incorporated herein by reference in their entirety.

### A. Samples and Cell Preparations

In particular embodiments, the provided methods are used in connection with isolating, selecting, and/or enriching cells from a biological sample to generate one or more input compositions of enriched cells, e.g., T cells. In some embodiments, the provided methods include isolation of cells or compositions thereof from biological samples, such as those obtained from or derived from a subject, such as one having a particular disease or condition or in need of a cell therapy or to which cell therapy will be administered. In some embodiments, features of the subject, for example as described in Section I-A and I-A.1 above, to be treated are determined or obtained and used a input to machine learning models provided herein. In some aspects, the subject is a human, such as a subject who is a patient in need of a particular therapeutic intervention, such as the adoptive cell therapy for which cells are being isolated, processed, and/or engineered. Accordingly, the cells in some embodiments are primary cells, e.g., primary human cells. The samples include tissue, fluid, and other samples taken directly from the subject. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca⁺⁺/Mg⁺⁺ free PBS. In certain embodiments, components of a blood cell sample are removed and the cells directly resuspended in culture media.

In some embodiments, the preparation methods include steps for freezing, e.g., cryopreserving, the cells, either before or after isolation, selection and/or enrichment and/or incubation for transduction and engineering, and/or after cultivation and/or harvesting of the engineered cells. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. In some embodiments, the cells are frozen, e.g., cryofrozen or cryopreserved, in media and/or solution with a final concentration of or of about 12.5%, 12.0%, 11.5%, 11.0%, 10.5%, 10.0%, 9.5%, 9.00%, 8.5%, 8.0%, 7.5%, 7.0%, 6.5%, 6.0%, 5.5%, or 5.0% DMSO, or between 1% and 15%, between 6% and 12%, between 5% and 10%, or between 6% and 8% DMSO. In particular embodiments, the cells are frozen, e.g., cryofrozen or cryopreserved, in media and/or solution with a final concentration of or of about 5.0%, 4.5%, 4.0%, 3.5%, 3.0%, 2.5%, 2.0%, 1.5%, 1.25%, 1.0%, 0.75%, 0.5%, or 0.25% HSA, or between 0.1% and −5%, between 0.25% and 4%, between 0.5% and 2%, or between 1% and 2% HSA. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are generally then frozen to or to about −80° C. at a rate of or of about 1° per minute and stored in the vapor phase of a liquid nitrogen storage tank.

In some embodiments, isolation of the cells or populations includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components. In some embodiments, the methods include density-based cell separation methods, such as the preparation of white blood cells from peripheral blood by lysing the red blood cells and centrifugation through a Percoll or Ficoll gradient.

In some embodiments, at least a portion of the selection step includes incubation of cells with a selection reagent. The incubation with a selection reagent or reagents, e.g., as part of selection methods which may be performed using one or more selection reagents for selection of one or more different cell types based on the expression or presence in or on the cell of one or more specific molecules, such as surface markers, e.g., surface proteins, intracellular markers, or nucleic acid. In some embodiments, any known method using a selection reagent or reagents for separation based on such markers may be used. In some embodiments, the selection reagent or reagents result in a separation that is affinity- or immunoaffinity-based separation. For example, the selection in some aspects includes incubation with a reagent or reagents for separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

In some aspects of such processes, a volume of cells is mixed with an amount of a desired affinity-based selection reagent. The immunoaffinity-based selection can be carried out using any system or method that results in a favorable energetic interaction between the cells being separated and the molecule specifically binding to the marker on the cell, e.g., the antibody or other binding partner on the solid surface, e.g., particle. In some embodiments, methods are carried out using particles such as beads, e.g., magnetic beads, that are coated with a selection agent (e.g., antibody) specific to the marker of the cells. The particles (e.g., beads) can be incubated or mixed with cells in a container, such as a tube or bag, while shaking or mixing, with a constant cell density-to-particle (e.g., bead) ratio to aid in promoting energetically favored interactions. In other cases, the methods include selection of cells in which all or a portion of the selection is carried out in the internal cavity of a centrifugal chamber, for example, under centrifugal rotation. In some embodiments, incubation of cells with selection reagents, such as immunoaffinity-based selection reagents, is performed in a centrifugal chamber. In certain embodiments, the isolation or separation is carried out using a system, device, or apparatus described in International Patent Application, Publication Number WO2009/072003, or US 20110003380 A1. In one example, the system is a system as described in International Publication Number WO2016/073602.

In some embodiments, by conducting such selection steps or portions thereof (e.g., incubation with antibody-coated particles, e.g., magnetic beads) in the cavity of a centrifugal chamber, the user is able to control certain parameters, such as volume of various solutions, addition of solution during processing and timing thereof, which can provide advantages compared to other available methods. For example, the ability to decrease the liquid volume in the cavity during the incubation can increase the concentration of the particles (e.g., bead reagent) used in the selection, and thus the chemical potential of the solution, without affecting the total number of cells in the cavity. This in turn can enhance the pairwise interactions between the cells being processed and the particles used for selection. In some embodiments, carrying out the incubation step in the chamber, e.g., when associated with the systems, circuitry, and control as described herein, permits the user to effect agitation of the solution at desired time(s) during the incubation, which also can improve the interaction.

In some embodiments, at least a portion of the selection step is performed in a centrifugal chamber, which includes incubation of cells with a selection reagent. In some aspects of such processes, a volume of cells is mixed with an amount of a desired affinity-based selection reagent that is far less than is normally employed when performing similar selections in a tube or container for selection of the same number of cells and/or volume of cells according to manufacturer's instructions. In some embodiments, an amount of selection reagent or reagents that is/are no more than 5%, no more than 10%, no more than 15%, no more than 20%, no more than 25%, no more than 50%, no more than 60%, no more than 70% or no more than 80% of the amount of the same selection reagent(s) employed for selection of cells in a tube or container-based incubation for the same number of cells and/or the same volume of cells according to manufacturer's instructions is employed.

In some embodiments, for selection, e.g., immunoaffinity-based selection of the cells, the cells are incubated in the cavity of the chamber in a composition that also contains the selection buffer with a selection reagent, such as a molecule that specifically binds to a surface marker on a cell that it desired to enrich and/or deplete, but not on other cells in the composition, such as an antibody, which optionally is coupled to a scaffold such as a polymer or surface, e.g., bead, e.g., magnetic bead, such as magnetic beads coupled to monoclonal antibodies specific for CD4 and CD8. In some embodiments, as described, the selection reagent is added to cells in the cavity of the chamber in an amount that is substantially less than (e.g., is no more than 5%, 10%, 20%, 30%, 40%, 50%, 60%, 70% or 80% of the amount) as compared to the amount of the selection reagent that is typically used or would be necessary to achieve about the same or similar efficiency of selection of the same number of cells or the same volume of cells when selection is performed in a tube with shaking or rotation. In some embodiments, the incubation is performed with the addition of a selection buffer to the cells and selection reagent to achieve a target volume with incubation of the reagent of, for example, 10 mL to 200 mL, such as at least or about at least or about 10 mL, 20 mL, 30 mL, 40 mL, 50 mL, 60 mL, 70 mL, 80 mL, 90 mL, 100 mL, 150 mL or 200 mL. In some embodiments, the selection buffer and selection reagent are pre-mixed before addition to the cells. In some embodiments, the selection buffer and selection reagent are separately added to the cells. In some embodiments, the selection incubation is carried out with periodic gentle mixing condition, which can aid in promoting energetically favored interactions and thereby permit the use of less overall selection reagent while achieving a high selection efficiency.

In some embodiments, the total duration of the incubation with the selection reagent is from 5 minutes to 6 hours or from about 5 minutes to about 6 hours, such as 30 minutes to 3 hours, for example, at least or about at least 30 minutes, 60 minutes, 120 minutes or 180 minutes.

In some embodiments, the incubation generally is carried out under mixing conditions, such as in the presence of spinning, generally at relatively low force or speed, such as speed lower than that used to pellet the cells, such as from 600 rpm to 1700 rpm or from about 600 rpm to about 1700 rpm (e.g., at or about or at least 600 rpm, 1000 rpm, or 1500 rpm or 1700 rpm), such as at an RCF at the sample or wall of the chamber or other container of from 80 g to 100 g or from about 80 g to about 100 g (e.g., at or about or at least 80 g, 85 g, 90 g, 95 g, or 100 g). In some embodiments, the spin is carried out using repeated intervals of a spin at such low speed followed by a rest period, such as a spin and/or rest for 1, 2, 3, 4, 5, 6, 7, 8, 9, or 10 seconds, such as a spin at approximately 1 or 2 seconds followed by a rest for approximately 5, 6, 7, or 8 seconds.

In some embodiments, such process is carried out within the entirely closed system to which the chamber is integral. In some embodiments, this process (and in some aspects also one or more additional step, such as a previous wash step washing a sample containing the cells, such as an apheresis sample) is carried out in an automated fashion, such that the cells, reagent, and other components are drawn into and pushed out of the chamber at appropriate times and centrifugation effected, so as to complete the wash and binding step in a single closed system using an automated program.

In some embodiments, after the incubation and/or mixing of the cells and selection reagent and/or reagents, the incubated cells are subjected to a separation to select for cells based on the presence or absence of the particular reagent or reagents. In some embodiments, the separation is performed in the same closed system in which the incubation of cells with the selection reagent was performed. In some embodiments, after incubation with the selection reagents, incubated cells, including cells in which the selection reagent has bound are transferred into a system for immunoaffinity-based separation of the cells. In some embodiments, the system for immunoaffinity-based separation is or contains a magnetic separation column.

Such separation steps can be based on positive selection, in which the cells having bound the reagents, e.g., antibody or binding partner, are retained for further use, and/or negative selection, in which the cells having not bound to the reagent, e.g., antibody or binding partner, are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

In some embodiments, the process steps further include negative and/or positive selection of the incubated and cells, such as using a system or apparatus that can perform an affinity-based selection. In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker+) at a relatively higher level (marker^{high}) on the positively or negatively selected cells, respectively. Multiple rounds of the same selection step, e.g., positive or negative selection step, can be performed. In certain embodiments, the positively or negatively selected fraction subjected to the process for selection, such as by repeating a positive or negative selection step. In some embodiments, selection is repeated twice, three times, four times, five times, six times, seven times, eight times, nine times or more than nine times. In certain embodiments, the same selection is performed up to five times. In certain embodiments, the same selection step is performed three times.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types. In certain embodiments, one or more separation steps are repeated and/or performed more than once. In some embodiments, the positively or negatively selected fraction resulting from a separation step is subjected to the same separation step, such as by repeating the positive or negative selection step. In some embodiments, a single separation step is repeated and/or performed more than once, for example, to increase the yield of positively selected cells, to increase the purity of negatively selected cells, and/or to further remove the positively selected cells from the negatively selected fraction. In certain embodiments, one or more separation steps are performed and/or repeated two times, three times, four times, five times, six times, seven times, eight times, nine times, ten times, or more than ten times. In certain embodiments, the one or more selection steps are performed and/or repeated between one and ten times, between one and five times, or between three and five times. In certain embodiments, one or more selection steps are repeated three times.

For example, in some aspects, specific subpopulations of T cells, such as cells positive or expressing high levels of one or more surface markers, e.g., CD28+, CD62L+, CCR7+, CD27+, CD127+, CD4+, CD8+, CD45RA+, and/or CD45RO+ T cells, are isolated by positive or negative selection techniques. In some embodiments, such cells are selected by incubation with one or more antibody or binding partner that specifically binds to such markers. In some embodiments, the antibody or binding partner can be conjugated, such as directly or indirectly, to a solid support or matrix to effect selection, such as a magnetic bead or paramagnetic bead. For example, CD3+, CD28+ T cells can be positively selected using CD3/CD28 conjugated magnetic beads (e.g., DYNABEADS® M-450 CD3/CD28 T Cell Expander, and/or ExpACT® beads).

In some embodiments, T cells are separated from a PBMC sample by negative selection of markers expressed on non-T cells, such as B cells, monocytes, or other white blood cells, such as CD14. In some aspects, a CD4+ or CD8+ selection step is used to separate CD4+ helper and CD8+ cytotoxic T cells. Such CD4+ and CD8+ populations can be further sorted into sub-populations by positive or negative selection for markers expressed or expressed to a relatively higher degree on one or more naive, memory, and/or effector T cell subpopulations.

In some embodiments, CD8+ T cells are further enriched for or depleted of naive, central memory, effector memory, and/or central memory stem cells, such as by positive or negative selection based on surface antigens associated with the respective subpopulation. In some embodiments, enrichment for central memory T (TCM) cells is carried out to increase efficacy, such as to improve long-term survival, expansion, and/or engraftment following administration, which in some aspects is particularly robust in such sub-populations. See Terakura et al., (2012) Blood. 1:72-82; Wang et al. (2012) J Immunother. 35(9):689-701. In some embodiments, combining TCM-enriched CD8+ T cells and CD4+ T cells further enhances efficacy.

In embodiments, memory T cells are present in both CD62L+ and CD62L− subsets of CD8+ peripheral blood lymphocytes. PBMC can be enriched for or depleted of CD62L-CD8+ and/or CD62L+CD8+ fractions, such as using anti-CD8 and anti-CD62L antibodies.

In some embodiments, the enrichment for central memory T (TCM) cells is based on positive or high surface expression of CD45RO, CD62L, CCR7, CD28, CD3, and/or CD 127; in some aspects, it is based on negative selection for cells expressing or highly expressing CD45RA and/or granzyme B. In some aspects, isolation of a CD8+ population enriched for TCM cells is carried out by depletion of cells expressing CD4, CD14, CD45RA, and positive selection or enrichment for cells expressing CD62L. In one aspect, enrichment for central memory T (TCM) cells is carried out starting with a negative fraction of cells selected based on CD4 expression, which is subjected to a negative selection based on expression of CD14 and CD45RA, and a positive selection based on CD62L.

Such selections in some aspects are carried out simultaneously and in other aspects are carried out sequentially, in either order. In some aspects, the same CD4 expression-based selection step used in preparing the CD8+ T cell population or subpopulation, also is used to generate the CD4+ T cell population or sub-population, such that both the positive and negative fractions from the CD4-based separation are retained and used in subsequent steps of the methods, optionally following one or more further positive or negative selection steps. In some embodiments, the selection for the CD4+ T cell population and the selection for the CD8+ T cell population are carried out simultaneously. In some embodiments, the CD4+ T cell population and the selection for the CD8+ T cell population are carried out sequentially, in either order. In some embodiments, methods for selecting cells can include those as described in published U.S. App. No. US20170037369. In some embodiments, the selected CD4+ T cell population and the selected CD8+ T cell population may be combined subsequent to the selecting. In some aspects, the selected CD4+ T cell population and the selected CD8+ T cell population may be combined in a bioreactor bag as described herein. In some embodiments, the selected CD4+ T cell population and the selected CD8+ T cell population are separately processed, whereby the selected CD4+ T cell population is enriched in CD4+ T cells and incubated with a stimulatory reagent (e.g., anti-CD3/anti-CD28 magnetic beads), transduced with a viral vector encoding a recombinant protein (e.g., CAR) and cultivated under conditions to expand T cells and the selected CD8+ T cell population is enriched in CD8+ T cell and incubated with a stimulatory reagent (e.g., anti-CD3/anti-CD28 magnetic beads), transduced with a viral vector encoding a recombinant protein (e.g., CAR), such as the same recombinant protein as for engineering of the CD4+ T cells from the same donor, and cultivated under conditions to expand T cells, such as in accord with the provided methods.

In particular embodiments, a biological sample, e.g., a sample of PBMCs or other white blood cells, are subjected to selection of CD4+ T cells, where both the negative and positive fractions are retained. In certain embodiments, CD8+ T cells are selected from the negative fraction. In some embodiments, a biological sample is subjected to selection of CD8+ T cells, where both the negative and positive fractions are retained. In certain embodiments, CD4+ T cells are selected from the negative fraction.

In a particular example, a sample of PBMCs or other white blood cell sample is subjected to selection of CD4+ T cells, where both the negative and positive fractions are retained. The negative fraction then is subjected to negative selection based on expression of CD14 and CD45RA or CD19, and positive selection based on a marker characteristic of central memory T cells, such as CD62L or CCR7, where the positive and negative selections are carried out in either order.

CD4+ T helper cells may be sorted into naive, central memory, and effector cells by identifying cell populations that have cell surface antigens. CD4+ lymphocytes can be obtained by standard methods. In some embodiments, naive CD4+ T lymphocytes are CD45RO−, CD45RA+, CD62L+, or CD4+ T cells. In some embodiments, central memory CD4+ T cells are CD62L+ and CD45RO+. In some embodiments, effector CD4+ T cells are CD62L− and CD45RO−.

In one example, to enrich for CD4+ T cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immunomagnetic (or affinitymagnetic) separation techniques (reviewed in Methods in Molecular Medicine, vol. 58: Metastasis Research Protocols, Vol. 2: Cell Behavior In Vitro and In Vivo, p 17-25 Edited by: S. A. Brooks and U. Schumacher © Humana Press Inc., Totowa, NJ).

In some aspects, the incubated sample or composition of cells to be separated is incubated with a selection reagent containing small, magnetizable or magnetically responsive material, such as magnetically responsive particles or microparticles, such as paramagnetic beads (e.g., such as Dynabeads or MACS® beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. Many well-known magnetically responsive materials for use in magnetic separation methods are known, e.g., those described in Molday, U.S. Pat. No. 4,452,773, and in European Patent Specification EP 452342 B, which are hereby incorporated by reference. Colloidal sized particles, such as those described in Owen U.S. Pat. No. 4,795,698, and Liberti et al., U.S. Pat. No. 5,200,084 also may be used.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In certain embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies.

In some aspects, separation is achieved in a procedure in which the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In some embodiments, the affinity-based selection is via magnetic-activated cell sorting (MACS) (Miltenyi Biotech, Auburn, CA). Magnetic Activated Cell Sorting (MACS), e.g., CliniMACS systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, magnetizable particles or antibodies conjugated to cleavable linkers, etc. In some embodiments, the magnetizable particles are biodegradable.

In some embodiments, the isolation and/or selection results in one or more input compositions of enriched T cells, e.g., CD3+ T cells, CD4+ T cells, and/or CD8+ T cells. In some embodiments, two or more separate input composition are isolated, selected, enriched, or obtained from a single biological sample. In some embodiments, separate input compositions are isolated, selected, enriched, and/or obtained from separate biological samples collected, taken, and/or obtained from the same subject.

In some embodiments, features of the one or more input compositions are assessed, for example as described in Sections I-A and I-A-2. In some embodiments, the features are cell phenotypes. In some embodiments, the cell phenotypes, are quantified to provide a number, percentage, proportion, and/or ratio of cells having an attribute in the input composition. In some embodiments, the features are used as input to machine learning models provided herein.

In certain embodiments, the one or more input compositions is or includes a composition of enriched T cells that includes at least 60%, at least 65%, at least 70%, at least 75%, at least 80%, at least 85%, at least 90%, at least 95%, at least 98%, at least 99%, at least 99.5%, at least 99.9%, or at or at about 100% CD3+ T cells. In particular embodiment, the input composition of enriched T cells consists essentially of CD3+ T cells.

In certain embodiments, the one or more input compositions is or includes a composition of enriched CD4+ T cells that includes at least 60%, at least 65%, at least 70%, at least 75%, at least 80%, at least 85%, at least 90%, at least 95%, at least 98%, at least 99%, at least 99.5%, at least 99.9%, or at or at about 100% CD4+ T cells. In certain embodiments, the input composition of CD4+ T cells includes less than 40%, less than 35%, less than 30%, less than 25%, less than 20%, less than 15%, less than 10%, less than 5%, less than 1%, less than 0.1%, or less than 0.01% CD8+ T cells, and/or contains no CD8+ T cells, and/or is free or substantially free of CD8+ T cells. In some embodiments, the composition of enriched T cells consists essentially of CD4+ T cells.

In certain embodiments, the one or more compositions is or includes a composition of CD8+ T cells that is or includes at least 60%, at least 65%, at least 70%, at least 75%, at least 80%, at least 85%, at least 90%, at least 95%, at least 98%, at least 99%, at least 99.5%, at least 99.9%, or at or at about 100% CD8+ T cells. In certain embodiments, the composition of CD8+ T cells contains less than 40%, less than 35%, less than 30%, less than 25%, less than 20%, less than 15%, less than 10%, less than 5%, less than 1%, less than 0.1%, or less than 0.01% CD4+ T cells, and/or contains no CD4+ T cells, and/or is free of or substantially free of CD4+ T cells. In some embodiments, the composition of enriched T cells consists essentially of CD8+ T cells.

In some embodiments, the one or more input compositions of enriched T cells are frozen, e.g., cryopreserved and/or cryofrozen, after isolation, selection and/or enrichment. In some embodiments, the one or more input compositions of frozen e.g., cryopreserved and/or cryofrozen, prior to any steps of incubating, activating, stimulating, engineering, transducing, transfecting, cultivating, expanding, harvesting, and/or formulating the composition of cells. In particular embodiments, the one or more cryofrozen input compositions are stored, e.g., at or at about −80° C., for between 12 hours and 7 days, between 24 hours and 120 hours, or between 2 days and 5 days. In particular embodiments, the one or more cryofrozen input compositions are stored at or at about −80° C., for an amount of time of less than 10 days, 9 days, 8 days, 7 days, 6 days, or 5 days, 4 days, 3 days, 2 days, or 1 day. In some embodiments, the one or more cryofrozen input compositions are stored at or at about −80° C., for or for about 1 day, 2 days, 3 days, 4 days, 5 days, or 6 days.

### B. Activation and Stimulation of Cells

In order to achieve sufficient therapeutic doses of T cell compositions, T cells are often subject to one or more rounds of stimulation, activation and/or expansion. T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041, each of which is incorporated herein by reference in its entirety. T cells modified to express a CAR (e.g., an anti-BCMA CAR) can be activated and expanded before and/or after the T cells are modified. In addition, T cells may be contacted with one or more agents that modulate the PI3K cell signaling pathway before, during, and/or after activation and/or expansion. In one embodiment, T cells manufactured by the methods contemplated herein undergo one, two, three, four, or five or more rounds of activation and expansion, each of which may include one or more agents that modulate the PI3K cell signaling pathway.

In some embodiments, the provided methods are used in connection with incubating cells under stimulating conditions. In some embodiments, the stimulating conditions include conditions that activate or stimulate, and/or are capable of activating or stimulating a signal in the cell, e.g., a CD4+ T cell or CD8+ T cell, such as a signal generated from a TCR and/or a coreceptor. In some embodiments, the stimulating conditions include one or more steps of culturing, cultivating, incubating, activating, propagating the cells with and/or in the presence of a stimulatory reagent, e.g., a reagent that activates or stimulates, and/or is capable of activating or stimulating a signal in the cell. In some embodiments, the stimulatory reagent stimulates and/or activates a TCR and/or a coreceptor. In particular embodiments, the stimulatory reagent is a reagent described in Section II-B-1.

In certain embodiments, one or more compositions of enriched T cells are incubated under stimulating conditions prior to genetically engineering the cells, e.g., transfecting and/or transducing the cell such as by a technique provided in Section II-C. In particular embodiments, one or more compositions of enriched T cells are incubated under stimulating conditions after the one or more compositions have been isolated, selected, enriched, or obtained from a biological sample. In particular embodiments, the one or more compositions are input compositions. In particular embodiments, the one or more input compositions have been previously cryofrozen and stored, and are thawed prior to the incubation.

In certain embodiments, the one or more compositions of enriched T cells are or include two separate compositions, e.g., separate input compositions, of enriched T cells. In particular embodiments, two separate compositions of enriched T cells, e.g., two separate compositions of enriched T cells selected, isolated, and/or enriched from the same biological sample, are separately incubated under stimulating conditions. In certain embodiments, the two separate compositions include a composition of enriched CD4+ T cells. In particular embodiments, the two separate compositions include a composition of enriched CD8+ T cells. In some embodiments, two separate compositions of enriched CD4+ T cells and enriched CD8+ T cells are separately incubated under stimulating conditions.

In some embodiments, a single composition of enriched T cells is incubated under stimulating conditions. In certain embodiments, the single composition is a composition of enriched CD4+ T cells. In some embodiments, the single composition is a composition of enriched CD4+ and CD8+ T cells that have been combined from separate compositions prior to the incubation.

In some embodiments, the composition of enriched CD4+ T cells that is incubated under stimulating conditions includes at least 60%, at least 65%, at least 70%, at least 75%, at least 80%, at least 85%, at least 90%, at least 95%, at least 98%, at least 99%, at least 99.5%, at least 99.9%, or at or at about 100% CD4+ T cells. In certain embodiments, the composition of enriched CD4+ T cells that is incubated under stimulating conditions includes less than 40%, less than 35%, less than 30%, less than 25%, less than 20%, less than 15%, less than 10%, less than 5%, less than 1%, less than 0.1%, or less than 0.01% CD8+ T cells, and/or contains no CD8+ T cells, and/or is free or substantially free of CD8+ T cells.

In some embodiments, the composition of enriched CD8+ T cells that is incubated under stimulating conditions includes at least 60%, at least 65%, at least 70%, at least 75%, at least 80%, at least 85%, at least 90%, at least 95%, at least 98%, at least 99%, at least 99.5%, at least 99.9%, or at or at about 100% CD8+ T cells. In certain embodiments, the composition of enriched CD8+ T cells that is incubated under stimulating conditions includes less than 40%, less than 35%, less than 30%, less than 25%, less than 20%, less than 15%, less than 10%, less than 5%, less than 1%, less than 0.1%, or less than 0.01% CD4+ T cells, and/or contains no CD4+ T cells, and/or is free or substantially free of CD4+ T cells.

In some embodiments, separate compositions of enriched CD4+ and CD8+ T cells are combined into a single composition and are incubated under stimulating conditions. In certain embodiments, separate stimulated compositions of enriched CD4+ and enriched CD8+ T cells are combined into a single composition after the incubation has been performed and/or completed. In some embodiments, separate stimulated compositions of stimulated CD4+ and stimulated CD8+ T cells are separately processed after the incubation has been performed and/or completed, whereby the stimulated CD4+ T cell population (e.g., incubated with stimulatory an anti-CD3/anti-CD28 magnetic bead stimulatory reagent) is transduced with a viral vector encoding a recombinant protein (e.g., CAR) and cultivated under conditions to expand T cells and the stimulated CD8+ T cell population (e.g., incubated with stimulatory an anti-CD3/anti-CD28 magnetic bead stimulatory reagent) is transduced with a viral vector encoding a recombinant protein (e.g., CAR), such as the same recombinant protein as for engineering of the CD4+ T cells from the same donor, and cultivated under conditions to expand T cells, such as in accord with the provided methods.

In some embodiments, the incubation under stimulating conditions can include culture, cultivation, stimulation, activation, propagation, including by incubation in the presence of stimulating conditions, for example, conditions designed to induce proliferation, expansion, activation, and/or survival of cells in the population, to mimic antigen exposure, and/or to prime the cells for genetic engineering, such as for the introduction of a recombinant antigen receptor. In particular embodiments, the stimulating conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some aspects, the stimulation and/or incubation under stimulating conditions is carried out in accordance with techniques such as those described in U.S. Pat. No. 6,040,177 to Riddell et al., Klebanoff et al. (2012) J Immunother. 35(9): 651-660, Terakura et al. (2012) Blood. 1:72-82, and/or Wang et al. (2012) J Immunother. 35(9):689-701.

In some embodiments, the cells, e.g., T cells, compositions of cells, and/or cell populations, such as CD4⁺ and CD8⁺ T cells or compositions, populations, or subpopulations thereof, are expanded by adding to the culture-initiating composition feeder cells, such as non-dividing peripheral blood mononuclear cells (PBMCs) (e.g., such that the resulting population of cells contains at least about 5, 10, 20, or 40 or more PBMC feeder cells for each T lymphocyte in the initial population to be expanded); and incubating the culture (e.g., for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells.

In one embodiment, a costimulatory ligand is presented on an antigen presenting cell (e.g., an aAPC, dendritic cell, B cell, and the like) that specifically binds a cognate costimulatory molecule on a T cell, thereby providing a signal which, in addition to the primary signal provided by, for instance, binding of a TCR/CD3 complex, mediates a desired T cell response. Suitable costimulatory ligands include, but are not limited to, CD7, B7-1 (CD80), B7-2 (CD86), PD-L 1, PD-L2, 4-1BBL, OX40L, inducible costimulatory ligand (ICOS-L), intercellular adhesion molecule (ICAM), CD30L, CD40, CD70, CD83, HLA-G, MICA, MICB, HVEM, lymphotoxin beta receptor, ILT3, ILT4, an agonist or antibody that binds Toll ligand receptor, and a ligand that specifically binds with B7-H3.

In a particular embodiment, a costimulatory ligand comprises an antibody or antigen binding fragment thereof that specifically binds to a costimulatory molecule present on a T cell, including but not limited to, CD27, CD28, 4-IBB, OX40, CD30, CD40, PD-1, 1COS, lymphocyte function-associated antigen 1 (LFA-1), CD7, LIGHT, NKG2C, B7-H3, and a ligand that specifically binds with CD83.

Suitable costimulatory ligands further include target antigens, which may be provided in soluble form or expressed on APCs or aAPCs that bind engineered TCRs or CARs expressed on modified T cells.

In some embodiments, the stimulating conditions include temperature suitable for the growth of human T lymphocytes, for example, at least about 25 degrees Celsius, generally at least about 30 degrees, and generally at or about 37 degrees Celsius. In some embodiments, a temperature shift is effected during culture, such as from 37 degrees Celsius to 35 degrees Celsius. Optionally, the incubation may further comprise adding non-dividing EBV-transformed lymphoblastoid cells (LCL) as feeder cells. LCL can be irradiated with gamma rays in the range of about 6000 to 10,000 rads. The LCL feeder cells in some aspects is provided in any suitable amount, such as a ratio of LCL feeder cells to initial T lymphocytes of at least about 10:1.

In embodiments, populations of CD4⁺ and CD8⁺ that are antigen specific can be obtained by stimulating naive or antigen specific T lymphocytes with antigen. For example, antigen-specific T cell lines or clones can be generated to cytomegalovirus antigens by isolating T cells from infected subjects and stimulating the cells in vitro with the same antigen. Naive T cells may also be used.

In particular embodiments, the stimulating conditions include incubating, culturing, and/or cultivating the cells with a stimulatory reagent. In particular embodiments, the stimulatory reagent is a reagent described in Section II-B-1. In certain embodiments, the stimulatory reagent contains or includes a bead. An exemplary stimulatory reagent is or includes anti-CD3/anti-CD28 magnetic beads. In certain embodiments, the start and/or initiation of the incubation, culturing, and/or cultivating cells under stimulating conditions occurs when the cells come into contact with and/or are incubated with the stimulatory reagent. In particular embodiments, the cells are incubated prior to, during, and/or subsequent to genetically engineering the cells, e.g., introducing a recombinant polynucleotide into the cell such as by transduction or transfection. In various embodiments, a method for manufacturing T cells contemplated herein comprises activating a population of cells comprising T cells and expanding the population of T cells. T cell activation can be accomplished by providing a primary stimulation signal through the T cell TCR/CD3 complex or via stimulation of the CD2 surface protein and by providing a secondary costimulation signal through an accessory molecule, e.g., CD28.

The TCR/CD3 complex may be stimulated by contacting the T cell with a suitable CD3 binding agent, e.g., a CD3 ligand or an anti-CD3 monoclonal antibody. Illustrative examples of CD3 antibodies include, but are not limited to, OKT3, G19-4, BC3, and 64.1.

In certain embodiments, a CD2 binding agent may be used to provide a primary stimulation signal to the T cells. Illustrative examples of CD2 binding agents include, but are not limited to, CD2 ligands and anti-CD2 antibodies, e.g., the T11.3 antibody in combination with the T11.1 or T11.2 antibody (Meuer, S. C. et al. (1984) Cell 36:897-906) and the 9.6 antibody (which recognizes the same epitope as TI 1.1) in combination with the 9-1 antibody (Yang, S. Y. et al. (1986) J. Immunol. 137:1097-1100). Other antibodies which bind to the same epitopes as any of the above described antibodies can also be used. Additional antibodies, or combinations of antibodies, can be prepared and identified by standard techniques as disclosed elsewhere herein.

In addition to the primary stimulation signal provided through the TCR/CD3 complex, or via CD2, induction of T cell responses requires a second, costimulatory signal. In particular embodiments, a CD28 binding agent can be used to provide a costimulatory signal. Illustrative examples of CD28 binding agents include but are not limited to: natural CD 28 ligands, e.g., a natural ligand for CD28 (e.g., a member of the B7 family of proteins, such as B7-1(CD80) and B7-2 (CD86); and anti-CD28 monoclonal antibody or fragment thereof capable of crosslinking the CD28 molecule, e.g., monoclonal antibodies 9.3, B-T3, XR-CD28, KOLT-2, 15E8, 248.23.2, and EX5.3D10.

In one embodiment, the molecule providing the primary stimulation signal, for example a molecule which provides stimulation through the TCR/CD3 complex or CD2, and the costimulatory molecule are coupled to the same surface.

In certain embodiments, binding agents that provide stimulatory and costimulatory signals are localized on the surface of a cell. This can be accomplished by transfecting or transducing a cell with a nucleic acid encoding the binding agent in a form suitable for its expression on the cell surface or alternatively by coupling a binding agent to the cell surface/

In certain embodiments, the molecule providing the primary stimulation signal, for example a molecule which provides stimulation through the TCR/CD3 complex or CD2, and the costimulatory molecule are displayed on antigen presenting cells.

In one embodiment, the molecule providing the primary stimulation signal, for example a molecule which provides stimulation through the TCR/CD3 complex or CD2, and the costimulatory molecule are provided on separate surfaces.

In a certain embodiment, one of the binding agents that provide stimulatory and costimulatory signals is soluble (provided in solution) and the other agent(s) is provided on one or more surfaces.

In a particular embodiment, the binding agents that provide stimulatory and costimulatory signals are both provided in a soluble form (provided in solution).

In various embodiments, the methods for manufacturing T cells contemplated herein comprise activating T cells with anti-CD3 and anti-CD28 antibodies.

T cell compositions manufactured by the methods contemplated herein comprise T cells activated and/or expanded in the presence of one or more agents that inhibit a PI3K cell signaling pathway. T cells modified to express a CAR (e.g., an anti-BCMA CAR) can be activated and expanded before and/or after the T cells are modified. In particular embodiments, a population of T cells is activated, modified to express a CAR (e.g., an anti-BCMA CAR), and then cultured for expansion.

In one embodiment, T cells manufactured by the methods contemplated herein comprise an increased number of T cells expressing markers indicative of high proliferative potential and the ability to self-renew but that do not express or express substantially undetectable markers of T cell differentiation. These T cells may be repeatedly activated and expanded in a robust fashion and thereby provide an improved therapeutic T cell composition.

In one embodiment, a population of T cells activated and expanded in the presence of one or more agents that inhibit a PI3K cell signaling pathway is expanded at least 1.5 fold, at least 2 fold, at least 3 fold, at least 4 fold, at least 5 fold, at least 6 fold, at least 7 fold, at least 8 fold, at least 9 fold, at least 10 fold, at least 25 fold, at least 50 fold, at least 100 fold, at least 250 fold, at least 500 fold, at least 1000 fold, or more compared to a population of T cells activated and expanded without a PI3K inhibitor.

In one embodiment, a population of T cells characterized by the expression of markers young T cells are activated and expanded in the presence of one or more agents that inhibit a PI3K cell signaling pathway is expanded at least 1.5 fold, at least 2 fold, at least 3 fold, at least 4 fold, at least 5 fold, at least 6 fold, at least 7 fold, at least 8 fold, at least 9 fold, at least 10 fold, at least 25 fold, at least 50 fold, at least 100 fold, at least 250 fold, at least 500 fold, at least 1000 fold, or more compared the population of T cells activated and expanded without a PI3K inhibitor.

In one embodiment, expanding T cells activated by the methods contemplated herein further comprises culturing a population of cells comprising T cells for several hours (about 3 hours) to about 7 days to about 28 days or any hourly integer value in between. In certain embodiments, the T cell composition may be cultured for 14 days. In a particular embodiment, T cells are cultured for about 21 days. In certain embodiments, the T cell compositions are cultured for about 2-3 days. Several cycles of stimulation/activation/expansion may also be desired such that culture time of T cells can be 60 days or more.

In particular embodiments, conditions appropriate for T cell culture include an appropriate media (e.g., Minimal Essential Media or RPMI Media 1640 or, X-vivo 15, (Lonza)) and one or more factors necessary for proliferation and viability including, but not limited to serum (e.g., fetal bovine or human serum), interleukin-2 (IL-2), insulin, IFN-α, IL-4, IL-7, IL-21, GM-CSF, IL-10, IL-12, IL-15, TGFα, and TNF-α or any other additives suitable for the growth of cells known to the skilled artisan.

Further illustrative examples of cell culture media include, but are not limited to RPMI 1640, Clicks, AIM-V, DMEM, MEM, a-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells.

Illustrative examples of other additives for T cell expansion include, but are not limited to, surfactant, plasmanate, pH buffers such as HEPES, and reducing agents such as N-acetyl cysteine and 2-mercaptoethanol.

Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% C02).

In particular embodiments, PBMCs or isolated T cells are contacted with a stimulatory agent and costimulatory agent, such as anti-CD3 and anti-CD28 antibodies, generally attached to a bead or other surface, in a culture medium with appropriate cytokines, such as IL-2, IL-7, and/or IL-15.

In other embodiments, artificial APC (aAPC) may be made by engineering K562, U937, 721.221, T2, and C1R cells to direct the stable expression and secretion, of a variety of costimulatory molecules and cytokines. In a particular embodiment K32 or U32 aAPCs are used to direct the display of one or more antibody-based stimulatory molecules on the AAPC cell surface. Populations of T cells can be expanded by aAPCs expressing a variety of costimulatory molecules including, but not limited to, CD137L (4-1BBL), CD134L (OX40L), and/or CD80 or CD86. Finally, the aAPCs provide an efficient platform to expand genetically modified T cells and to maintain CD28 expression on CD8 T cells. aAPCs provided in WO 03/057171 and US2003/0147869 are hereby incorporated by reference in their entirety.

In some embodiments, the composition of enriched T cells are incubated at a ratio of stimulatory reagent and/or beads, e.g., anti-CD3/anti-CD28 magnetic beads, to cells at or at about 3:1, 2.5:1, 2:1, 1.5:1, 1.25:1, 1.2:1, 1.1:1, 1:1, 0.9:1, 0.8:1, 0.75:1, 0.67:1, 0.5:1, 0.3:1, or 0.2:1. In particular embodiments, the ratio of stimulatory reagent and/or beads to cells is between 2.5:1 and 0.2:1, between 2:1 and 0.5:1, between 1.5:1 and 0.75:1, between 1.25:1 and 0.8:1, between 1.1:1 and 0.9:1. In particular embodiments, the ratio of stimulatory reagent to cells is about 1:1 or is 1:1.

In particular embodiments, incubating the cells at a ratio of less than 3:1 or less than 3 stimulatory reagents, e.g., anti-CD3/anti-CD28 magnetic beads. per cell, such as a ratio of 1:1, reduces the amount of cell death that occurs during the incubation, e.g., such as by activation-induced cell death. In some embodiments, the cells are incubated with the stimulatory reagent, e.g., anti-CD3/anti-CD28 magnetic beads, at a ratio of beads to cells of less than 3 (or 3:1 or less than 3 beads per cell). In particular embodiments, incubating the cells at a ratio of less than 3:1 or less than 3 beads per cell, such as a ratio of 1:1, reduces the amount of cell death that occurs during the incubation, e.g., such as by activation-induced cell death.

In particular embodiments, the composition of enriched T cells is incubated with the stimulatory reagent, e.g., anti-CD3/anti-CD28 magnetic beads, at a ratio of less than 3:1 stimulatory reagents and/or beads per cell, such as a ratio of 1:1, and at least 50%, at least 60%, at least 70%, at least 75%, at least 80%, at least 85%, at least 90%, at least 95%, at least 99%, or at least 99.9% of the T cells survive, e.g., are viable and/or do not undergo necrosis, programed cell death, or apoptosis, during or at least 1 day, 2 days, 3 days, 4 days, 5 days, 6 days, 7 days, or more than 7 days after the incubation is complete. In particular embodiments, the composition of enriched T cells is incubated with the stimulatory reagent at a ratio of less than 3:1 stimulatory reagents and/or beads per cell, e.g., a ratio of 1:1, and less than 50%, less than 40%, less than 30%, less than 25%, less than 20%, less than 15%, less than 10%, less than 5%, less than 1%, less than 0.1% or less than 0.01% of the cells undergo activation induced cell death during the incubation.

In certain embodiments, the composition of enriched T cells is incubated with the stimulatory reagent, e.g., anti-CD3/anti-CD28 magnetic beads, at a ratio of less than 3:1 beads per cell, e.g., a ratio of 1:1, and the cells of the composition have at least 10%, at least 20%, at least 30%, at least 40%, at least 50%, at least 60%, at least 70%, at least 80%, at least 90%, at least 95%, at least 100%, at least 150%, at least 1-fold, at least 2-fold, at least 3-fold, at least 4-fold, at least 5-fold, at least 10-fold, at least 25-Fold, at least 50-fold, or at least 100-fold greater survival as compared to cells undergoing an exemplary and/or alternative process where the composition of enriched T cells in incubated with the stimulatory reagent at a ratio of 3:1 or greater.

In some embodiments, the composition of enriched T cells incubated with the stimulatory reagent comprises from 1.0×10⁵ cells/mL to 1.0×10⁸ cells/mL or from about 1.0×10⁵ cells/mL to about 1.0×10⁸ cells/mL, such as at least or about at least or about 1.0×10⁵ cells/mL, 5×10⁵ cells/mL, 1×10⁶ cells/mL, 5×10⁶ cells/mL, 1×10⁷ cells/mL, 5×10⁷ cells/mL or 1×10⁸ cells/mL. In some embodiments, the composition of enriched T cells incubated with the stimulatory reagent comprises about 0.5×10⁶ cells/mL, 1×10⁶ cells/mL, 1.5×10⁶ cells/mL, 2×10⁶ cells/mL, 2.5×10⁶ cells/mL, 3×10⁶ cells/mL, 3.5×10⁶ cells/mL, 4×10⁶ cells/mL, 4.5×10⁶ cells/mL, 5×10⁶ cells/mL, 5.5×10⁶ cells/mL, 6×10⁶ cells/mL, 6.5×10⁶ cells/mL, 7×10⁶ cells/mL, 7.5×10⁶ cells/mL, 8×10⁶ cells/mL, 8.5×10⁶ cells/mL, 9×10⁶ cells/mL, 9.5×10⁶ cells/mL, or 10×10⁶ cells/mL, such as about 2.4×10⁶ cells/mL.

In some embodiments, the composition of enriched T cells is incubated with the stimulatory reagent at a temperature from about 25 to about 38° C., such as from about 30 to about 37° C., for example at or about 37° C.±2° C. In some embodiments, the composition of enriched T cells is incubated with the stimulatory reagent at a CO₂ level from about 2.5% to about 7.5%, such as from about 4% to about 6%, for example at or about 5%±0.5%. In some embodiments, the composition of enriched T cells is incubated with the stimulatory reagent at a temperature of or about 37° C. and/or at a CO₂ level of or about 5%.

In particular embodiments, the stimulating conditions include incubating, culturing, and/or cultivating a composition of enriched T cells with and/or in the presence of one or more cytokines. In particular embodiments, the one or more cytokines are recombinant cytokines. In some embodiments, the one or more cytokines are human recombinant cytokines. In certain embodiments, the one or more cytokines bind to and/or are capable of binding to receptors that are expressed by and/or are endogenous to T cells. In particular embodiments, the one or more cytokines is or includes a member of the 4-alpha-helix bundle family of cytokines. In some embodiments, members of the 4-alpha-helix bundle family of cytokines include, but are not limited to, interleukin-2 (IL-2), interleukin-4 (IL-4), interleukin-7 (IL-7), interleukin-9 (IL-9), interleukin 12 (IL-12), interleukin 15 (IL-15), granulocyte colony-stimulating factor (G-CSF), and granulocyte-macrophage colony-stimulating factor (GM-CSF). In some embodiments, the one or more cytokines is or includes IL-15. In particular embodiments, the one or more cytokines is or includes IL-7. In particular embodiments, the one or more cytokines is or includes IL-2. In some embodiments, the stimulating conditions include incubating composition of enriched T cells, such as enriched CD4+ T cells or enriched CD8+ T cells, in the presence of a stimulatory reagent, e.g., anti-CD3/anti-CD28 magnetic beads, as described and in the presence or one or more recombinant cytokines.

In particular embodiments, the composition of enriched CD4+ T cells are incubated with IL-2, e.g., recombinant IL-2. Without wishing to be bound by theory, particular embodiments contemplate that CD4+ T cells that are obtained from some subjects do not produce, or do not sufficiently produce, IL-2 in amounts that allow for growth, division, and expansion throughout the process for generating a composition of output cells, e.g., engineered cells suitable for use in cell therapy. In some embodiments, incubating a composition of enriched CD4+ T cells under stimulating conditions in the presence of recombinant IL-2 increases the probability or likelihood that the CD4+ T cells of the composition will continue to survive, grow, expand, and/or activate during the incubation step and throughout the process. In some embodiments, incubating the composition of enriched CD4+ T cells in the presence of recombinant IL-2 increases the probability and/or likelihood that an output composition of enriched CD4+ T cells, e.g., engineered CD4+ T cells suitable for cell therapy, will be produced from the composition of enriched CD4+ T cells by at least 0.5%, at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 30%, at least 40%, at least 50%, at least 60%, at least 70%, at least 80%, at least 90%, at least 95%, at least 100%, at least 150%, at least 1-fold, at least 2-fold, at least 3-fold, at least 4-fold, at least 5-fold, at least 10-fold, at least 25-fold, at least 50-fold, or at least 100-fold as compared to an alternative and/or exemplary method that does not incubate the composition of enriched CD4+ T cells in the presence of recombinant IL-2.

In certain embodiments, the amount or concentration of the one or more cytokines are measured and/or quantified with International Units (IU). International units may be used to quantify vitamins, hormones, cytokines, vaccines, blood products, and similar biologically active substances. In some embodiments, IU are or include units of measure of the potency of biological preparations by comparison to an international reference standard of a specific weight and strength e.g., WHO 1st International Standard for Human IL-2, 86/504. International Units are the only recognized and standardized method to report biological activity units that are published and are derived from an international collaborative research effort. In particular embodiments, the IU for composition, sample, or source of a cytokine may be obtained through product comparison testing with an analogous WHO standard product. For example, in some embodiments, the IU/mg of a composition, sample, or source of human recombinant IL-2, IL-7, or IL-15 is compared to the WHO standard IL-2 product (NIBSC code: 86/500), the WHO standard IL-17 product (NIBSC code: 90/530) and the WHO standard IL-15 product (NIBSC code: 95/554), respectively.

In some embodiments, the biological activity in IU/mg is equivalent to (ED₅₀ in ng/ml)⁻¹×10⁶. In particular embodiments, the ED₅₀ of recombinant human IL-2 or IL-15 is equivalent to the concentration required for the half-maximal stimulation of cell proliferation (XTT cleavage) with CTLL-2 cells. In certain embodiments, the ED₅₀ of recombinant human IL-7 is equivalent to the concentration required for the half-maximal stimulation for proliferation of PHA-activated human peripheral blood lymphocytes. Details relating to assays and calculations of IU for IL-2 are discussed in Wadhwa et al., Journal of Immunological Methods (2013), 379 (1-2): 1-7; and Gearing and Thorpe, Journal of Immunological Methods (1988), 114 (1-2): 3-9; details relating to assays and calculations of IU for IL-15 are discussed in Soman et al. Journal of Immunological Methods (2009) 348 (1-2): 83-94; hereby incorporated by reference in their entirety.

In particular embodiments, a composition of enriched CD8+ T cells is incubated under stimulating conditions in the presence of IL-2 and/or IL-15. In certain embodiments, a composition of enriched CD4+ T cells is incubated under stimulating conditions in the presence of IL-2, IL-7, and/or IL-15. In some embodiments, the IL-2, IL-7, and/or IL-15 are recombinant. In certain embodiments, the IL-2, IL-7, and/or IL-15 are human. In particular embodiments, the one or more cytokines are or include human recombinant IL-2, IL-7, and/or IL-15. In some aspects, the incubation of the enriched T cell composition also includes the presence of a stimulatory reagent, e.g., anti-CD3/anti-CD28 magnetic beads.

In some embodiments, the cells are incubated with a cytokine, e.g., a recombinant human cytokine, at a concentration of between 1 IU/ml and 1,000 IU/ml, between 10 IU/ml and 50 IU/ml, between 50 IU/ml and 100 IU/ml, between 100 IU/ml and 200 IU/ml, between 100 IU/ml and 500 IU/ml, between 250 IU/ml and 500 IU/ml, or between 500 IU/ml and 1,000 IU/ml.

In some embodiments, a composition of enriched T cells is incubated with IL-2, e.g., human recombinant IL-2, at a concentration between 1 IU/ml and 200 IU/ml, between 10 IU/ml and 200 IU/ml, between 10 IU/ml and 100 IU/ml, between 50 IU/ml and 150 IU/ml, between 80 IU/ml and 120 IU/ml, between 60 IU/ml and 90 IU/ml, or between 70 IU/ml and 90 IU/ml. In particular embodiments, the composition of enriched T cells is incubated with recombinant IL-2 at a concentration at or at about 50 IU/ml, 55 IU/ml, 60 IU/ml, 65 IU/ml, 70 IU/ml, 75 IU/ml, 80 IU/ml, 85 IU/ml, 90 IU/ml, 95 IU/ml, 100 IU/ml, 110 IU/ml, 120 IU/ml, 130 IU/ml, 140 IU/ml, or 150 IU/ml. In some embodiments, the composition of enriched T cells is incubated in the presence of or of about 85 IU/ml recombinant IL-2. In some embodiments, the composition incubated with recombinant IL-2 is enriched for a population of T cells, e.g., CD4+ T cells and/or CD8+ T cells. In some embodiments, the population of T cells is a population of CD4+ T cells. In some embodiments, the composition of enriched T cells is a composition of enriched CD8+ T cells. In particular embodiments, the composition of enriched T cells is enriched for CD8+ T cells, where CD4+ T cells are not enriched for and/or where CD4+ T cells are negatively selected for or depleted from the composition. In some embodiments, the composition of enriched T cells is a composition of enriched CD4+ T cells. In particular embodiments, the composition of enriched T cells is enriched for CD4+ T cells, where CD8+ T cells are not enriched for and/or where CD8+ T cells are negatively selected for or depleted from the composition. In some embodiments, an enriched CD4+ T cell composition incubated with recombinant IL-2 may also be incubated with recombinant IL-7 and/or recombinant IL-15, such as in amounts described. In some embodiments, an enriched CD8+ T cell composition incubated with recombinant IL-2 may also be incubated with recombinant IL-15, such as in amounts described.

In some embodiments, a composition of enriched T cells is incubated with recombinant IL-7, e.g., human recombinant IL-7, at a concentration between 100 IU/ml and 2,000 IU/ml, between 500 IU/ml and 1,000 IU/ml, between 100 IU/ml and 500 IU/ml, between 500 IU/ml and 750 IU/ml, between 750 IU/ml and 1,000 IU/ml, or between 550 IU/ml and 650 IU/ml. In particular embodiments, the composition of enriched T cells is incubated with recombinant IL-7 at a concentration at or at about 50 IU/ml, 100 IU/ml, 150 IU/ml, 200 IU/ml, 250 IU/ml, 300 IU/ml, 350 IU/ml, 400 IU/ml, 450 IU/ml, 500 IU/ml, 550 IU/ml, 600 IU/ml, 650 IU/ml, 700 IU/ml, 750 IU/ml, 800 IU/ml, 750 IU/ml, 750 IU/ml, 750 IU/ml, or 1,000 IU/ml. In particular embodiments, the composition of enriched T cells is incubated in the presence of or of about 600 IU/ml of recombinant IL-7. In some embodiments, the composition incubated with recombinant IL-7 is enriched for a population of T cells, e.g., CD4+ T cells. In some embodiments, an enriched CD4+ T cell composition incubated with recombinant IL-7 may also be incubated with recombinant IL-2 and/or recombinant IL-15, such as in amounts described. In particular embodiments, the composition of enriched T cells is enriched for CD4+ T cells, where CD8+ T cells are not enriched for and/or where CD8+ T cells are negatively selected for or depleted from the composition. In some embodiments, an enriched CD8+ T cell composition is not incubated with recombinant IL-7.

In some embodiments, a composition of enriched T cells is incubated with recombinant IL-15, e.g., human recombinant IL-15, at a concentration between 0.1 IU/ml and 100 IU/ml, between 1 IU/ml and 100 IU/ml, between 1 IU/ml and 50 IU/ml, between 5 IU/ml and 25 IU/ml, between 25 IU/ml and 50 IU/ml, between 5 IU/ml and 15 IU/ml, or between 10 IU/ml and 100 IU/ml. In particular embodiments, the composition of enriched T cells is incubated with recombinant IL-15 at a concentration at or at about 1 IU/ml, 2 IU/ml, 3 IU/ml, 4 IU/ml, 5 IU/ml, 6 IU/ml, 7 IU/ml, 8 IU/ml, 9 IU/ml, 10 IU/ml, 11 IU/ml, 12 IU/ml, 13 IU/ml, 14 IU/ml, 15 IU/ml, 20 IU/ml, 25 IU/ml, 30 IU/ml, 40 IU/ml, or 50 IU/ml. In some embodiments, the composition of enriched T cells is incubated in or in about 10 IU/ml of recombinant IL-15. In some embodiments, the composition incubated with recombinant IL-15 is enriched for a population of T cells, e.g., CD4+ T cells and/or CD8+ T cells. In some embodiments, the population of T cells is a population of CD4+ T cells. In some embodiments, the composition of enriched T cells is a composition of enriched CD8+ T cells. In particular embodiments, the composition of enriched T cells is enriched for CD8+ T cells, where CD4+ T cells are not enriched for and/or where CD4+ T cells are negatively selected for or depleted from the composition. In some embodiments, the composition of enriched T cells is a composition of enriched CD4+ T cells. In particular embodiments, the composition of enriched T cells is enriched for CD4+ T cells, where CD8+ T cells are not enriched for and/or where CD8+ T cells are negatively selected for or depleted from the composition. In some embodiments, an enriched CD4+ T cell composition incubated with recombinant IL-15 may also be incubated with recombinant IL-7 and/or recombinant IL-2, such as in amounts described. In some embodiments, an enriched CD8+ T cell composition incubated with recombinant IL-15 may also be incubated with recombinant IL-2, such as in amounts described.

In particular embodiments, the cells, such as enriched CD4+ T cells and/or enriched CD8+ T cells, are incubated with the stimulatory reagent in the presence of one or more antioxidants. In some embodiments, antioxidants include, but are not limited to, one or more antioxidants comprise a tocopherol, a tocotrienol, alpha-tocopherol, beta-tocopherol, gamma-tocopherol, delta-tocopherol, alpha-tocotrienol, beta-tocotrienol, alpha-tocopherolquinone, Trolox (6-hydroxy-2,5,7,8-tetramethylchroman-2-carboxylic acid), butylated hydroxyanisole (BHA), butylated hydroxytoluene (BHT), a flavonoids, an isoflavone, lycopene, beta-carotene, selenium, ubiquinone, luetin, S-adenosylmethionine, glutathione, taurine, N-acetyl cysteine (NAC), citric acid, L-carnitine, BHT, monothioglycerol, ascorbic acid, propyl gallate, methionine, cysteine, homocysteine, gluthatione, cystamine and cysstathionine, and/or glycine-glycine-histidine. In some aspects, the incubation of the enriched T cell composition, such as enriched CD4+ T cells and/or enriched CD8+ T cells, with an antioxidant also includes the presence of a stimulatory reagent, e.g., anti-CD3/anti-CD28 magnetic beads, and one or more recombinant cytokines, such as described.

In some embodiments, the one or more antioxidants is or includes a sulfur containing oxidant. In certain embodiments, a sulfur containing antioxidant may include thiol-containing antioxidants and/or antioxidants which exhibit one or more sulfur moieties, e.g., within a ring structure. In some embodiments, the sulfur containing antioxidants may include, for example, N-acetyl cysteine (NAC) and 2,3-dimercaptopropanol (DMP), L-2-oxo-4-thiazolidinecarboxylate (OTC) and lipoic acid. In particular embodiments, the sulfur containing antioxidant is a glutathione precursor. In some embodiments, the glutathione precursor is a molecule which may be modified in one or more steps within a cell to derived glutathione. In particular embodiments, a glutathione precursor may include, but is not limited to N-acetyl cysteine (NAC), L-2-oxothiazolidine-4-carboxylic acid (Procysteine), lipoic acid, S-allyl cysteine, or methylmethionine sulfonium chloride.

In some embodiments, incubating the cells, such as enriched CD4+ T cells and/or enriched CD8+ T cells, under stimulating conditions includes incubating the cells in the presence of one or more antioxidants. In particular embodiments, the cells are stimulated with the stimulatory reagent in the presence of one or more antioxidants. In some embodiments, the cells are incubated in the presence of between 1 ng/ml and 100 ng/ml, between 10 ng/ml and 1 μg/ml, between 100 ng/ml and 10 μg/ml, between 1 μg/ml and 100 μg/ml, between 10 μg/ml and 1 mg/ml, between 100 μg/ml and 1 mg/ml, between 1 500 μg/ml and 2 mg/ml, 500 μg/ml and 5 mg/ml, between 1 mg/ml and 10 mg/ml, or between 1 mg/ml and 100 mg/ml of the one or more antioxidants. In some embodiments, the cells are incubated in the presence of or of about 1 ng/ml, 10 ng/ml, 100 ng/ml, 1 μg/ml, 10 μg/ml, 100 μg/ml, 0.2 mg/ml, 0.4 mg/ml, 0.6 mg/ml, 0.8 mg/ml, 1 mg/ml, 2 mg/ml, 3 mg/ml, 4 mg/ml, 5 mg/ml, 10 mg/ml, 20 mg/ml, 25 mg/ml, 50 mg/ml, 100 mg/ml, 200 mg/ml, 300 mg/ml, 400 mg/ml, 500 mg/ml of the one or more antioxidant. In some embodiments, the one or more antioxidants is or includes a sulfur containing antioxidant. In particular embodiments, the one or more antioxidants is or includes a glutathione precursor.

In some embodiments, the one or more antioxidants is or includes N-acetyl cysteine (NAC). In some embodiments, incubating the cells, such as enriched CD4+ T cells and/or enriched CD8+ T cells, under stimulating conditions includes incubating the cells in the presence of NAC. In particular embodiments, the cells are stimulated with the stimulatory reagent in the presence of NAC. In some embodiments, the cells are incubated in the presence of between 1 ng/ml and 100 ng/ml, between 10 ng/ml and 1 μg/ml, between 100 ng/ml and 10 μg/ml, between 1 μg/ml and 100 μg/ml, between 10 μg/ml and 1 mg/ml, between 100 μg/ml and 1 mg/ml, between 1-500 μg/ml and 2 mg/ml, 500 μg/ml and 5 mg/ml, between 1 mg/ml and 10 mg/ml, or between 1 mg/ml and 100 mg/ml of NAC. In some embodiments, the cells are incubated in the presence of or of about 1 ng/ml, 10 ng/ml, 100 ng/ml, 1 μg/ml, 10 μg/ml, 100 μg/ml, 0.2 mg/ml, 0.4 mg/ml, 0.6 mg/ml, 0.8 mg/ml, 1 mg/ml, 2 mg/ml, 3 mg/ml, 4 mg/ml, 5 mg/ml, 10 mg/ml, 20 mg/ml, 25 mg/ml, 50 mg/ml, 100 mg/ml, 200 mg/ml, 300 mg/ml, 400 mg/ml, 500 mg/ml of NAC. In some embodiments, the cells are incubated with or with about 0.8 mg/ml.

In particular embodiments, incubating the composition of enriched T cells, such as enriched CD4+ T cells and/or enriched CD8+ T cells, in the presence of one or more antioxidants, e.g., NAC, reduces the activation in the cells as compared to cells that are incubated in alternative and/or exemplary processes without the presence of antioxidants. In certain embodiments, the reduced activation is measured by the expression of one or more activation markers in the cell. In certain embodiments, markers of activation include, but are not limited to, increased intracellular complexity (e.g., as determined by measuring side scatter (SSC), increased cell size (e.g., as determined by measuring cell diameter and/or forward scatter (FSC), increased expression of CD27, and/or decreased expression of CD25. In some embodiments, the cells of the composition have negative, reduced, or low expression and/or degree of markers of activation when examined during or after the incubation, engineering, transduction, transfection, expansion, or formulation, or during or after any stage of the process occurring after the incubation. In some embodiments the cells of the composition have negative, reduced, or low expression and/or degree of markers of activation after the process is completed. In particular embodiments, the cells of the output composition have negative, reduced, or low expression and/or degree of markers of activation.

In some embodiments, flow cytometry is used to determine relative size of cells. In particular embodiments, the FSC and SSC parameters are used to analyze cells and distinguish the cells from one another based off of size and internal complexity. In particular embodiments, a particle or bead of a known size can be measured as a standard to determine the actual size of cells. In some embodiments, flow cytometry is used in combination with a stain, e.g., a labeled antibody, to measure or quantify the expression of a surface protein, such as a marker of activation, e.g., CD25 or CD27.

In some embodiments, the composition of enriched T cells, such as enriched CD4+ T cells and/or enriched CD8+ T cells, is incubated in the presence of one or more antioxidants e.g., NAC, and the cell diameter reduced by at least 0.25 μm, 0.5 μm, 0.75 μm, 1.0 μm, 1.5 μm, 2 μm, 2.5 μm, 3 μm, 3.5 μm, 4 μm, 4.5 μm, 5 μm, or more than 5 μm as compared to cells incubated in an alternative and/or exemplary process where the incubation is not performed in the presence of an antioxidant. In particular embodiments, the composition of enriched T cells is incubated in the presence of one or more antioxidants e.g., NAC, and the cell size, as measured by the FSC is reduced by at least 1%, at least 5%, at least 10%, at least 20%, at least 30%, at least 40%, at least 50%, at least 60%, at least 70%, at least 75%, at least 80%, at least 85%, or at least 90% as compared to cells incubated in an alternative and/or exemplary process where the incubation is not performed in the presence of an antioxidant.

In some embodiments, the composition of enriched T cells, such as enriched CD4+ T cells and/or enriched CD8+ T cells, is incubated in the presence of one or more antioxidants e.g., NAC, and the intracellular complexity, as measured by the SSC, is reduced by at least 1%, at least 5%, at least 10%, at least 20%, at least 30%, at least 40%, at least 50%, at least 60%, at least 70%, at least 75%, at least 80%, at least 85%, or at least 90% as compared to cells incubated in an alternative and/or exemplary process where the incubation is not performed in the presence of an antioxidant.

In particular embodiments, the composition of enriched T cells, such as enriched CD4+ T cells and/or enriched CD8+ T cells, is incubated in the presence of one or more antioxidants e.g., NAC, and the expression of CD27, e.g., as measured by the flow cytometry, is reduced by at least 1%, at least 5%, at least 10%, at least 20%, at least 30%, at least 40%, at least 50%, at least 60%, at least 70%, at least 75%, at least 80%, at least 85%, at least 90%, at least 95%, or at least 99% as compared to cells incubated in an alternative and/or exemplary process where the incubation is not performed in the presence of an antioxidant.

In certain embodiments, the composition of enriched T cells, such as enriched CD4+ T cells and/or enriched CD8+ T cells, is incubated in the presence of one or more antioxidants, e.g., NAC, and the expression of CD25, e.g., as measured by the flow cytometry, is increased by at least 1%, at least 5%, at least 10%, at least 20%, at least 30%, at least 40%, at least 50%, at least 60%, at least 70%, at least 75%, at least 80%, at least 85%, at least 90%, at least 100%, at least 150%, at least 1-fold, at least 2-fold, at least 3-fold, at least 4-fold, at least 5-fold, at least 10-fold, at least 25-Fold, at least 50-fold, or at least 100-fold as compared to cells incubated in an alternative and/or exemplary process where the incubation is not performed in the presence of an antioxidant.

In particular embodiments, incubating the composition of enriched T cells, such as enriched CD4+ T cells and/or enriched CD8+ T cells, in the presence of one or more antioxidants, e.g., NAC, increases the expansion, e.g., during the incubation or cultivation step or stage as described in Section II-D. In some embodiments, a composition of enriched cells achieves a 2-fold, a 2.5 fold, a 3 fold, a 3.5 fold, a 4 fold, a 4.5 fold a 5 fold, a 6 fold, a 7 fold, an 8 fold, a nine fold, a 10-fold, or greater than a 10 fold expansion within 14 days, 12 days, 10 days, 9 days, 8 days, 7 days, 6 days, 5 days, 4 days, or within 3 days of the start of the cultivation. In some embodiments, the composition of enriched T cells is incubated in the presence of one or more antioxidants and the cells of the compositions undergo at least 10%, at least a 20%, at least a 30%, at least a 40%, at least a 50%, at least a 60%, at least a 70%, at least a 75%, at least an 80%, at least an 85%, at least a 90%, at least a 100%, at least a 150%, at least a 1-fold, at least a 2-fold, at least a 3-fold, at least a 4-fold, at least a 5-fold, at least a 10-fold faster rate of expansion during the cultivation than cultivated cells that were incubated in an alternative and/or exemplary process where the incubation is not performed in the presence of an antioxidant.

In particular embodiments, incubating the composition of enriched cells, such as enriched CD4+ T cells and/or enriched CD8+ T cells, in the presence of one or more antioxidants, e.g., NAC, reduces the amount of cell death, e.g., by apoptosis. In some embodiments, the composition of enriched T cells is incubated in the presence of a one or more antioxidants, e.g., NAC, and at least 50%, at least 60%, at least 70%, at least 75%, at least 80%, at least 85%, at least 90%, at least 95%, at least 99%, or at least 99.9% of the cells survive, e.g., do not undergo apoptosis, during or at least 1 day, 2 days, 3 days, 4 days, 5 days, 6 days, 7 days, or more than 7 days after the incubation is complete. In some embodiments, the composition is incubated in the presence of one or more antioxidants, e.g., NAC, and the cells of the composition have at least 10%, at least 20%, at least 30%, at least 40%, at least 50%, at least 60%, at least 70%, at least 80%, at least 90%, at least 95%, at least 100%, at least 150%, at least 1-fold, at least 2-fold, at least 3-fold, at least 4-fold, at least 5-fold, at least 10-fold, at least 25-Fold, at least 50-fold, or at least 100-fold greater survival as compared to cells undergoing an exemplary and/or alternative process where cells are not incubated in the presence or one or more antioxidants.

In particular embodiments, the composition of enriched T cells, such as enriched CD4+ T cells and/or enriched CD8+ T cells, is incubated in the presence of one or more antioxidants e.g., NAC, and caspase expression, e.g., caspase 3 expression, is reduced by at least 1%, at least 5%, at least 10%, at least 20%, at least 30%, at least 40%, at least 50%, at least 60%, at least 70%, at least 75%, at least 80%, at least 85%, at least 90%, at least 95%, or at least 99% as compared to cells incubated in an alternative and/or exemplary process where the incubation is not performed in the presence of an antioxidant.

In some embodiments, the compositions or cells, such as enriched CD4+ T cells and/or enriched CD8+ T cells, are incubated in the presence of stimulating conditions or a stimulatory agent, such as described. Such conditions include those designed to induce proliferation, expansion, activation, and/or survival of cells in the population, to mimic antigen exposure, and/or to prime the cells for genetic engineering, such as for the introduction of a recombinant antigen receptor. Exemplary stimulatory reagents, such as anti-CD3/anti-CD28 magnetic beads, are described below. The incubation with the stimulatory reagent may also be carried out in the presence of one or more stimulatory cytokine, such as in the presence of one or more of recombinant IL-2, recombinant IL-7 and/or recombinant IL-15 and/or in the presence of at least one antioxidant such as NAC, such as described above. In some embodiments, a composition of enriched CD4+ T cells are incubated under stimulatory conditions with a stimulatory agent, recombinant IL-2, recombinant IL-7, recombinant IL-15 and NAC, such as in amounts as described. In some embodiments, a composition of enriched CD8+ T cells are incubated under stimulatory conditions with a stimulatory agent, recombinant IL-2, recombinant IL-15 and NAC, such as in amounts as described.

In some embodiments, the conditions for stimulation and/or activation can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some aspects, incubation is carried out in accordance with techniques such as those described in U.S. Pat. No. 6,040,177 to Riddell et al., Klebanoff et al. (2012) J Immunother. 35(9): 651-660, Terakura et al. (2012) Blood. 1:72-82, and/or Wang et al. (2012) J Immunother. 35(9):689-701.

In some embodiments, at least a portion of the incubation in the presence of one or more stimulating conditions or a stimulatory agents is carried out in the internal cavity of a centrifugal chamber, for example, under centrifugal rotation, such as described in International Publication Number WO2016/073602. In some embodiments, at least a portion of the incubation performed in a centrifugal chamber includes mixing with a reagent or reagents to induce stimulation and/or activation. In some embodiments, cells, such as selected cells, are mixed with a stimulating condition or stimulatory agent in the centrifugal chamber. In some aspects of such processes, a volume of cells is mixed with an amount of one or more stimulating conditions or agents that is far less than is normally employed when performing similar stimulations in a cell culture plate or other system.

In some embodiments, the stimulating agent is added to cells in the cavity of the chamber in an amount that is substantially less than (e.g., is no more than 5%, 10%, 20%, 30%, 40%, 50%, 60%, 70% or 80% of the amount) as compared to the amount of the stimulating agent that is typically used or would be necessary to achieve about the same or similar efficiency of selection of the same number of cells or the same volume of cells when selection is performed without mixing in a centrifugal chamber, e.g., in a tube or bag with periodic shaking or rotation. In some embodiments, the incubation is performed with the addition of an incubation buffer to the cells and stimulating agent to achieve a target volume with incubation of the reagent of, for example, about 10 mL to about 200 mL, or about 20 mL to about 125 mL, such as at least or about at least or about 10 mL, 20 mL, 30 mL, 40 mL, 50 mL, 60 mL, 70 mL, 80 mL, 90 mL, 100 mL, 105 mL, 110 mL, 115 mL, 120 mL, 125 mL, 130 mL, 135 mL, 140 mL, 145 mL, 150 mL, 160 mL, 170 mL, 180 mL, 190 mL, or 200 mL. In some embodiments, the incubation buffer and stimulating agent are pre-mixed before addition to the cells. In some embodiments, the incubation buffer and stimulating agent are separately added to the cells. In some embodiments, the stimulating incubation is carried out with periodic gentle mixing condition, which can aid in promoting energetically favored interactions and thereby permit the use of less overall stimulating agent while achieving stimulating and activation of cells.

In some embodiments, the incubation generally is carried out under mixing conditions, such as in the presence of spinning, generally at relatively low force or speed, such as speed lower than that used to pellet the cells, such as from 600 rpm to 1700 rpm or from about 600 rpm to about 1700 rpm (e.g., at or about or at least 600 rpm, 1000 rpm, or 1500 rpm or 1700 rpm), such as at an RCF at the sample or wall of the chamber or other container of from 80 g to 100 g or from about 80 g to about 100 g (e.g., at or about or at least 80 g, 85 g, 90 g, 95 g, or 100 g). In some embodiments, the spin is carried out using repeated intervals of a spin at such low speed followed by a rest period, such as a spin and/or rest for 1, 2, 3, 4, 5, 6, 7, 8, 9, or 10 seconds, such as a spin at approximately 1 or 2 seconds followed by a rest for approximately 5, 6, 7, or 8 seconds.

In some embodiments, the total duration of the incubation, e.g., with the stimulating agent, is between or between about 1 hour and 96 hours, 1 hour and 72 hours, 1 hour and 48 hours, 4 hours and 36 hours, 8 hours and 30 hours, 18 hours and 30 hours, or 12 hours and 24 hours, such as at least or about at least or about 6 hours, 12 hours, 18 hours, 24 hours, 36 hours or 72 hours. In some embodiments, the further incubation is for a time between or about between 1 hour and 48 hours, 4 hours and 36 hours, 8 hours and 30 hours or 12 hours and 24 hours, inclusive.

In some embodiments, the cells are cultured, cultivated, and/or incubated under stimulating conditions prior to and/or during a step for introducing a polynucleotide, e.g., a polynucleotide encoding a recombinant receptor, to the cells, e.g., by transduction and/or transfection, such as described by Section II-C. In certain embodiments the cells are cultured, cultivated, and/or incubated under stimulating conditions for an amount of time between 30 minutes and 2 hours, between 1 hour and 8 hours, between 1 hour and 6 hours, between 6 hours and 12 hours, between 12 hours and 18 hours, between 16 hours and 24 hours, between 12 hours and 36 hours, between 24 hours and 48 hours, between 24 hours and 72 hours, between 42 hours and 54 hours, between 60 hours and 120 hours between 96 hours and 120 hours, between 90 hours and between 1 days and 7 days, between 3 days and 8 days, between 1 day and 3 days, between 4 days and 6 days, or between 4 days and 5 days prior to the genetic engineering. In some embodiments, the cells are incubated for or for about 2 days prior to the engineering.

In certain embodiments, the cells are incubated with and/or in the presence of the stimulatory reagent prior to and/or during genetically engineering the cells. In certain embodiments the cells are incubated with and/or in the presence of the stimulatory reagent for an amount of time between 12 hours and 36 hours, between 24 hours and 48 hours, between 24 hours and 72 hours, between 42 hours and 54 hours, between 60 hours and 120 hours between 96 hours and 120 hours, between 90 hours and between 2 days and 7 days, between 3 days and 8 days, between 1 day and 8 days, between 4 days and 6 days, or between 4 days and 5 days. In particular embodiments, the cells are cultured, cultivated, and/or incubated under stimulating conditions prior to and/or during genetically engineering the cells for an amount of time of less than 10 days, 9 days, 8 days, 7 days, 6 days, or 5 days, 4 days, or for an amount of time less than 168 hours, 162 hours, 156 hours, 144 hours, 138 hours, 132 hours, 120 hours, 114 hours, 108 hours, 102 hours, or 96 hours. In particular embodiments, the cells are incubated with and/or in the presence of the stimulatory reagent for or for about 4 days, 5 days, 6 days, or 7 days. In some embodiments, the cells are incubated with and/or in the presence of the stimulatory reagent for or for about 4 days. In particular embodiments, the cells are incubated with and/or in the presence of the stimulatory reagent for or for about 5 days. In certain embodiments, the cells are incubated with and/or in the presence of the stimulatory reagent for less than 7 days.

In some embodiments, incubating the cells under stimulating conditions includes incubating the cells with a stimulatory reagent that is described in Section II-B-1. In some embodiments, the stimulatory reagent contains or includes a bead, such as a paramagnetic bead, and the cells are incubated with the stimulatory reagent at a ratio of less than 3:1 (beads:cells), such as a ratio of 1:1. In particular embodiments, the cells are incubated with the stimulatory reagent in the presence of one or more cytokines and/or one or more antioxidants. In some embodiments, a composition of enriched CD4+ T cells is incubated with the stimulatory reagent at a ratio of 1:1 (beads:cells) in the presence of recombinant IL-2, IL-7, IL-15, and NAC. In certain embodiments, a composition of enriched CD8+ T cells is incubated with the stimulatory reagent at a ratio of 1:1 (beads:cells) in the presence of recombinant IL-2, IL-15, and NAC. In some embodiments, the stimulatory reagent is removed and/or separated from the cells at, within, or within about 6 days, 5 days, or 4 days from the start or initiation of the incubation, e.g., from the time the stimulatory reagent is added to or contacted with the cells.

### 1. Stimulatory Reagents

In some embodiments, incubating a composition of enriched cells under stimulating conditions is or includes incubating and/or contacting the composition of enriched cells with a stimulatory reagent that is capable of activating and/or expanding T cells. In some embodiments, the stimulatory reagent is capable of stimulating and/or activating one or more signals in the cells. In some embodiments, the one or more signals are mediated by a receptor. In particular embodiments, the one or more signals are or are associated with a change in signal transduction and/or a level or amount of secondary messengers, e.g., cAMP and/or intracellular calcium, a change in the amount, cellular localization, confirmation, phosphorylation, ubiquitination, and/or truncation of one or more cellular proteins, and/or a change in a cellular activity, e.g., transcription, translation, protein degradation, cellular morphology, activation state, and/or cell division. In particular embodiments, the stimulatory reagent activates and/or is capable of activating one or more intracellular signaling domains of one or more components of a TCR complex and/or one or more intracellular signaling domains of one or more costimulatory molecules.

In certain embodiments, the stimulatory reagent contains a particle, e.g., a bead, that is conjugated or linked to one or more agents, e.g., biomolecules, that are capable of activating and/or expanding cells, e.g., T cells. In some embodiments, the one or more agents are bound to a bead. In some embodiments, the bead is biocompatible, i.e., composed of a material that is suitable for biological use. In some embodiments, the beads are non-toxic to cultured cells, e.g., cultured T cells. In some embodiments, the beads may be any particles which are capable of attaching agents in a manner that permits an interaction between the agent and a cell.

In some embodiments, a stimulatory reagent contains one or more agents that are capable of activating and/or expanding cells, e.g., T cells, that are bound to or otherwise attached to a bead, for example to the surface of the bead. In certain embodiments, the bead is a non-cell particle. In particular embodiments, the bead may include a colloidal particle, a microsphere, nanoparticle, a magnetic bead, or the like. In some embodiments the beads are agarose beads. In certain embodiments, the beads are sepharose beads.

In particular embodiments, the stimulatory reagent contains beads that are monodisperse. In certain embodiments, beads that are monodisperse comprise size dispersions having a diameter standard deviation of less than 5% from each other.

In some embodiments, the bead contains one or more agents, such as an agent that is coupled, conjugated, or linked (directly or indirectly) to the surface of the bead. In some embodiments, an agent as contemplated herein can include, but is not limited to, RNA, DNA, proteins (e.g., enzymes), antigens, polyclonal antibodies, monoclonal antibodies, antibody fragments, carbohydrates, lipids lectins, or any other biomolecule with an affinity for a desired target. In some embodiments, the desired target is a T cell receptor and/or a component of a T cell receptor. In certain embodiments, the desired target is CD3. In certain embodiment, the desired target is a T cell costimulatory molecule, e.g., CD28, CD137 (4-1-BB), OX40, or ICOS. The one or more agents may be attached directly or indirectly to the bead by a variety of methods known and available in the art. The attachment may be covalent, noncovalent, electrostatic, or hydrophobic and may be accomplished by a variety of attachment means, including for example, a chemical means, a mechanical means, or an enzymatic means. In some embodiments, a biomolecule (e.g., a biotinylated anti-CD3 antibody) may be attached indirectly to the bead via another biomolecule (e.g., anti-biotin antibody) that is directly attached to the bead.

In some embodiments, the stimulatory reagent contains a bead and one or more agents that directly interact with a macromolecule on the surface of a cell. In certain embodiments, the bead (e.g., a paramagnetic bead) interacts with a cell via one or more agents (e.g., an antibody) specific for one or more macromolecules on the cell (e.g., one or more cell surface proteins). In certain embodiments, the bead (e.g., a paramagnetic bead) is labeled with a first agent described herein, such as a primary antibody (e.g., an anti-biotin antibody) or other biomolecule, and then a second agent, such as a secondary antibody (e.g., a biotinylated anti-CD3 antibody) or other second biomolecule (e.g., streptavidin), is added, whereby the secondary antibody or other second biomolecule specifically binds to such primary antibodies or other biomolecule on the particle.

In some embodiments, the stimulatory reagent contains one or more agents (e.g., antibody) that is attached to a bead (e.g., a paramagnetic bead) and specifically binds to one or more of the following macromolecules on a cell (e.g., a T cell): CD2, CD3, CD4, CD5, CD8, CD25, CD27, CD28, CD29, CD31, CD44, CD45RA, CD45RO, CD54 (ICAM-1), CD127, MHCI, MHCII, CTLA-4, ICOS, PD-1, OX40, CD27L (CD70), 4-1BB (CD137), 4-1BBL, CD30L, LIGHT, IL-2R, IL-12R, IL-1R, IL-15R; IFN-gammaR, TNF-alphaR, IL-4R, IL-10R, CD18/CD11a (LFA-1), CD62L (L-selectin), CD29/CD49d (VLA-4), Notch ligand (e.g., Delta-like 1/4, Jagged 1/2, etc.), CCR1, CCR2, CCR3, CCR4, CCR5, CCR7, and CXCR3 or fragment thereof including the corresponding ligands to these macromolecules or fragments thereof. In some embodiments, an agent (e.g., antibody) attached to the bead specifically binds to one or more of the following macromolecules on a cell (e.g., a T cell): CD28, CD62L, CCR7, CD27, CD127, CD3, CD4, CD8, CD45RA, and/or CD45RO.

In some embodiments, one or more of the agents attached to the bead is an antibody. The antibody can include a polyclonal antibody, monoclonal antibody (including full length antibodies which have an immunoglobulin Fc region), antibody compositions with polyepitopic specificity, multispecific antibodies (e.g., bispecific antibodies, diabodies, and single-chain molecules, as well as antibody fragments (e.g., Fab, F(ab′)2, and Fv). In some embodiments, the stimulatory reagent is an antibody fragment (including antigen-binding fragment), e.g., a Fab, Fab′-SH, Fv, scFv, or (Fab′)2 fragment. It will be appreciated that constant regions of any isotype can be used for the antibodies contemplated herein, including IgG, IgM, IgA, IgD, and IgE constant regions, and that such constant regions can be obtained from any human or animal species (e.g., murine species). In some embodiments, the agent is an antibody that binds to and/or recognizes one or more components of a T cell receptor. In particular embodiments, the agent is an anti-CD3 antibody. In certain embodiments, the agent is an antibody that binds to and/or recognizes a co-receptor. In some embodiments, the stimulatory reagent comprises an anti-CD28 antibody. In some embodiments, the bead has a diameter of greater than about 0.001 μm, greater than about 0.01 μm, greater than about 0.1 μm, greater than about 1.0 μm, greater than about 10 μm, greater than about 50 μm, greater than about 100 μm or greater than about 1000 μm and no more than about 1500 μm. In some embodiments, the bead has a diameter of about 1.0 μm to about 500 μm, about 1.0 μm to about 150 μm, about 1.0 μm to about 30 μm, about 1.0 μm to about 10 μm, about 1.0 μm to about 5.0 μm, about 2.0 μm to about 5.0 μm, or about 3.0 μm to about 5.0 μm. In some embodiments, the bead has a diameter of about 3 μm to about 5 μm. In some embodiments, the bead has a diameter of at least or at least about or about 0.001 μm, 0.01 μm, 0.1 μm, 0.5 μm, 1.0 μm, 1.5 μm, 2.0 μm, 2.5 μm, 3.0 μm, 3.5 μm, 4.0 μm, 4.5 μm, 5.0 μm, 5.5 μm, 6.0 μm, 6.5 μm, 7.0 μm, 7.5 μm, 8.0 μm, 8.5 μm, 9.0 μm, 9.5 μm, 10 μm, 12 μm, 14 μm, 16 μm, 18 μm or 20 μm. In certain embodiments, the bead has a diameter of or about 4.5 μm. In certain embodiments, the bead has a diameter of or about 2.8 μm.

In some embodiments, the beads have a density of greater than 0.001 g/cm³, greater than 0.01 g/cm³, greater than 0.05 g/cm³, greater than 0.1 g/cm³, greater than 0.5 g/cm³, greater than 0.6 g/cm³, greater than 0.7 g/cm³, greater than 0.8 g/cm³, greater than 0.9 g/cm³, greater than 1 g/cm³, greater than 1.1 g/cm³, greater than 1.2 g/cm³, greater than 1.3 g/cm³, greater than 1.4 g/cm³, greater than 1.5 g/cm³, greater than 2 g/cm³, greater than 3 g/cm³, greater than 4 g/cm³, or greater than 5 g/cm³. In some embodiments, the beads have a density of between about 0.001 g/cm³ and about 100 g/cm³, about 0.01 g/cm³ and about 50 g/cm³, about 0.1 g/cm³ and about 10 g/cm³, about 0.1 g/cm³ and about 0.5 g/cm³, about 0.5 g/cm³ and about 1 g/cm³, about 0.5 g/cm³ and about 1.5 g/cm³, about 1 g/cm³ and about 1.5 g/cm³, about 1 g/cm³ and about 2 g/cm³, or about 1 g/cm³ and about 5 g/cm³. In some embodiments, the beads have a density of about 0.5 g/cm³, about 0.5 g/cm³, about 0.6 g/cm³, about 0.7 g/cm³, about 0.8 g/cm³, about 0.9 g/cm³, about 1.0 g/cm³, about 1.1 g/cm³, about 1.2 g/cm³, about 1.3 g/cm³, about 1.4 g/cm³, about 1.5 g/cm³, about 1.6 g/cm³, about 1.7 g/cm³, about 1.8 g/cm³, about 1.9 g/cm³, or about 2.0 g/cm³. In certain embodiments, the beads have a density of about 1.6 g/cm³. In particular embodiments, the beads or particles have a density of about 1.5 g/cm³. In certain embodiments, the particles have a density of about 1.3 g/cm³.

In certain embodiments, a plurality of the beads has a uniform density. In certain embodiments, a uniform density comprises a density standard deviation of less than 10%, less than 5%, or less than 1% of the mean bead density.

In some embodiments, the beads have a surface area of between about 0.001 m² per each gram of particles (m²/g) to about 1,000 m²/g, about 0.010 m²/g to about 100 m²/g, about 0.1 m²/g to about 10 m²/g, about 0.1 m²/g to about 1 m²/g, about 1 m²/g to about 10 m²/g, about 10 m²/g to about 100 m²/g, about 0.5 m²/g to about 20 m²/g, about 0.5 m²/g to about 5 m²/g, or about 1 m²/g to about 4 m²/g. In some embodiments, the particles or beads have a surface area of about 1 m²/g to about 4 m²/g.

In some embodiments, the bead contains at least one material at or near the bead surface that can be coupled, linked, or conjugated to an agent. In some embodiments, the bead is surface functionalized, i.e. comprises functional groups that are capable of forming a covalent bond with a binding molecule, e.g., a polynucleotide or a polypeptide. In particular embodiments, the bead comprises surface-exposed carboxyl, amino, hydroxyl, tosyl, epoxy, and/or chloromethyl groups. In particular embodiments, the beads comprise surface exposed agarose and/or sepharose. In certain embodiments, the bead surface comprises attached stimulatory reagents that can bind or attach binding molecules. In particular embodiments, the biomolecules are polypeptides. In some embodiments, the beads comprise surface exposed protein A, protein G, or biotin.

In some embodiments, the bead reacts in a magnetic field. In some embodiments, the bead is a magnetic bead. In some embodiments, the magnetic bead is paramagnetic. In particular embodiments, the magnetic bead is superparamagnetic. In certain embodiments, the beads do not display any magnetic properties unless they are exposed to a magnetic field.

In particular embodiments, the bead comprises a magnetic core, a paramagnetic core, or a superparamagnetic core. In some embodiments, the magnetic core contains a metal. In some embodiments, the metal can be, but is not limited to, iron, nickel, copper, cobalt, gadolinium, manganese, tantalum, zinc, zirconium or any combinations thereof. In certain embodiments, the magnetic core comprises metal oxides (e.g., iron oxides), ferrites (e.g., manganese ferrites, cobalt ferrites, nickel ferrites, etc.), hematite and metal alloys (e.g., CoTaZn). In some embodiments, the magnetic core comprises one or more of a ferrite, a metal, a metal alloy, an iron oxide, or chromium dioxide. In some embodiments, the magnetic core comprises elemental iron or a compound thereof. In some embodiments, the magnetic core comprises one or more of magnetite (Fe3O4), maghemite (γFe2O3), or greigite (Fe3S4). In some embodiments, the inner core comprises an iron oxide (e.g., Fe₃O₄).

In certain embodiments, the bead contains a magnetic, paramagnetic, and/or superparamagnetic core that is covered by a surface functionalized coat or coating. In some embodiments, the coat can contain a material that can include, but is not limited to, a polymer, a polysaccharide, a silica, a fatty acid, a protein, a carbon, agarose, sepharose, or a combination thereof. In some embodiments, the polymer can be a polyethylene glycol, poly (lactic-co-glycolic acid), polyglutaraldehyde, polyurethane, polystyrene, or a polyvinyl alcohol. In certain embodiments, the outer coat or coating comprises polystyrene. In particular embodiments, the outer coating is surface functionalized.

In some embodiments, the stimulatory reagent comprises a bead that contains a metal oxide core (e.g., an iron oxide core) and a coat, wherein the metal oxide core comprises at least one polysaccharide (e.g., dextran), and wherein the coat comprises at least one polysaccharide (e.g., amino dextran), at least one polymer (e.g., polyurethane) and silica. In some embodiments the metal oxide core is a colloidal iron oxide core. In certain embodiments, the one or more agents include an antibody or antigen-binding fragment thereof. In particular embodiments, the one or more agents include an anti-CD3 antibody and an anti-CD28 antibody or antigen-binding fragments thereof. In some embodiments, the stimulatory reagent comprises an anti-CD3 antibody, anti-CD28 antibody, and an anti-biotin antibody. In some embodiments, the stimulatory reagent comprises an anti-biotin antibody. In some embodiments, the bead has a diameter of about 3 μm to about 10 μm. In some embodiments, the bead has a diameter of about 3 μm to about 5 μm. In certain embodiments, the bead has a diameter of about 3.5 μm.

In some embodiments, the stimulatory reagent comprises one or more agents that are attached to a bead comprising a metal oxide core (e.g., an iron oxide inner core) and a coat (e.g., a protective coat), wherein the coat comprises polystyrene. In certain embodiments, the beads are monodisperse, paramagnetic (e.g., superparamagnetic) beads comprising a paramagnetic (e.g., superparamagnetic) iron core, e.g., a core comprising magnetite (Fe₃O₄) and/or maghemite (γFe₂O₃) c and a polystyrene coat or coating. In some embodiments, the bead is non-porous. In some embodiments, the beads contain a functionalized surface to which the one or more agents are attached. In certain embodiments, the one or more agents are covalently bound to the beads at the surface. In some embodiments, the one or more agents include an antibody or antigen-binding fragment thereof. In some embodiments, the one or more agents include an anti-CD3 antibody and an anti-CD28 antibody. In some embodiments, the stimulatory reagent is or comprises anti-CD3/anti-CD28 magnetic beads, In some embodiments, the one or more agents include an anti-CD3 antibody and/or an anti-CD28 antibody, and an antibody or antigen fragment thereof capable of binding to a labeled antibody (e.g., biotinylated antibody), such as a labeled anti-CD3 or anti-CD28 antibody. In certain embodiments, the beads have a density of about 1.5 g/cm³ and a surface area of about 1 m²/g to about 4 m²/g. In particular embodiments; the beads are monodisperse superparamagnetic beads that have a diameter of about 4.5 μm and a density of about 1.5 g/cm³. In some embodiments, the beads the beads are monodisperse superparamagnetic beads that have a mean diameter of about 2.8 μm and a density of about 1.3 g/cm³.

In some embodiments, the composition of enriched T cells is incubated with stimulatory reagent a ratio of beads to cells at or at about 3:1, 2.5:1, 2:1, 1.5:1, 1.25:1, 1.2:1, 1.1:1, 1:1, 0.9:1, 0.8:1, 0.75:1, 0.67:1, 0.5:1, 0.3:1, or 0.2:1. In particular embodiments, the ratio of beads to cells is between 2.5:1 and 0.2:1, between 2:1 and 0.5:1, between 1.5:1 and 0.75:1, between 1.25:1 and 0.8:1, between 1.1:1 and 0.9:1. In particular embodiments, the ratio of stimulatory reagent to cells is about 1:1 or is 1:1.

2. Removal of the Stimulatory Reagent from Cells

In certain embodiments, the stimulatory reagent, e.g., anti-CD3/anti-CD28 magnetic beads, is removed and/or separated from the cells. Without wishing to be bound by theory, particular embodiments contemplate that the binding and/or association between a stimulatory reagent and cells may, in some circumstances, be reduced over time during the incubation. In certain embodiments, one or more agents may be added to reduce the binding and/or association between the stimulatory reagent and the cells. In particular embodiments, a change in cell culture conditions, e.g., media temperature of pH, may reduce the binding and/or association between the stimulatory reagent and the cells. Thus, in some embodiments, the stimulatory reagent may be removed from an incubation, cell culture system, and/or a solution separately from the cells, e.g., without removing the cells from the incubation, cell culture system, and/or a solution as well.

Methods for removing stimulatory reagents (e.g., stimulatory reagents that are or contain particles such as bead particles or magnetizable particles) from cells are known. In some embodiments, the use of competing antibodies, such as non-labeled antibodies, can be used, which, for example, bind to a primary antibody of the stimulatory reagent and alter its affinity for its antigen on the cell, thereby permitting for gentle detachment. In some cases, after detachment, the competing antibodies may remain associated with the particle (e.g., bead particle) while the unreacted antibody is or may be washed away and the cell is free of isolating, selecting, enriching and/or activating antibody. Exemplary of such a reagent is DETACHaBEAD™ (Friedl et al. 1995; Entschladen et al. 1997). In some embodiments, particles (e.g., bead particles) can be removed in the presence of a cleavable linker (e.g., DNA linker), whereby the particle-bound antibodies are conjugated to the linker (e.g., CELLection, Dynal). In some cases, the linker region provides a cleavable site to remove the particles (e.g., bead particles) from the cells after isolation, for example, by the addition of DNase or other releasing buffer. In some embodiments, other enzymatic methods can also be employed for release of a particle (e.g., bead particle) from cells. In some embodiments, the particles (e.g., bead particles or magnetizable particles) are biodegradable.

In some embodiments, the stimulatory reagent is magnetic, paramagnetic, and/or superparamagnetic, and/or contains a bead that is magnetic, paramagnetic, and/or superparamagnetic, and the stimulatory reagent may be removed from the cells by exposing the cells to a magnetic field. Examples of suitable equipment containing magnets for generating the magnetic field include DynaMag CTS (Thermo Fisher), Magnetic Separator (Takara) and EasySep Magnet (Stem Cell Technologies).

In particular embodiments, the stimulatory reagent is removed or separated from the cells prior to the completion of the provided methods, e.g., prior to harvesting, collecting, and/or formulating engineered cells produced by the methods provided herein. In some embodiments, the stimulatory reagent is removed and/or separated from the cells prior to engineering, e.g., transducing or transfecting, the cells. In particular embodiments, the stimulatory reagent is removed and/or separated from the cells after the step of engineering the cells. In certain embodiments, the stimulatory reagent is removed prior to the cultivation of the cells, e.g., prior to the cultivation of the engineered, e.g., transfected or transduced, cells under conditions to promote proliferation and/or expansion.

In certain embodiments, the stimulatory reagent is separated and/or removed from the cells after an amount of time. In particular embodiments, the amount of time is an amount of time from the start and/or initiation of the incubation under stimulating conditions. In particular embodiments the start of the incubation is considered at or at about the time the cells are contacted with the stimulatory reagent and/or a media or solution containing the stimulatory reagent. In particular embodiments, the stimulatory reagent is removed or separated from the cells within or within about 10 days, 9 days, 8 days, 7 days, 6 days, 5 days, 4 days, 3 days, or 2 days after the start or initiation of the incubation. In particular embodiments, the stimulatory reagent is removed and/or separated from the cells at or at about 9 days, 8 days, 7 days, 6 days, 5 days, 4 days, 3 days, or 2 days after the start or initiation of the incubation. In certain embodiments, the stimulatory reagent is removed and/or separated from the cells at or at about 168 hours, 162 hours, 156 hours, 144 hours, 138 hours, 132 hours, 120 hours, 114 hours, 108 hours, 102 hours, or 96 hours after the start or initiation of the incubation. In particular embodiments, the stimulatory reagent is removed and/or separated from the cells at or at about 5 days after the start and/or initiation of the incubation. In some embodiments, the stimulatory reagent is removed and/or separated from the cells at or at about 4 days after the start and/or initiation of the incubation.

### C. Engineering Cells

In some embodiments, the provided methods involve administering to a subject having a disease or condition cells expressing a recombinant antigen receptor. Various methods for the introduction of genetically engineered components, e.g., recombinant receptors, e.g., CARs or TCRs, are well known and may be used with the provided methods and compositions. Exemplary methods include those for transfer of nucleic acids encoding the receptors, including via viral, e.g., retroviral or lentiviral, transduction, transposons, and electroporation.

Among the cells expressing the receptors and administered by the provided methods are engineered cells. The genetic engineering generally involves introduction of a nucleic acid encoding the recombinant or engineered component into a composition containing the cells, such as by retroviral transduction, transfection, or transformation.

In some embodiments, the methods provided herein are used in association with engineering one or more compositions of enriched T cells. In certain embodiments, the engineering is or includes the introduction of a polynucleotide, e.g., a recombinant polynucleotide encoding a recombinant protein. In particular embodiments, the recombinant proteins are recombinant receptors, such as any described in Section III.

Introduction of the nucleic acid molecules encoding the recombinant protein, such as recombinant receptor, in the cell may be carried out using any of a number of known vectors. Such vectors include viral and non-viral systems, including lentiviral and gammaretroviral systems, as well as transposon-based systems such as PiggyBac or Sleeping Beauty-based gene transfer systems. Exemplary methods include those for transfer of nucleic acids encoding the receptors, including via viral, e.g., retroviral or lentiviral, transduction, transposons, and electroporation. In some embodiments, the engineering produces one or more engineered compositions of enriched T cells.

In certain embodiments, one or more compositions of enriched T cells are engineered, e.g., transduced or transfected, prior to cultivating the cells, e.g., under conditions that promote proliferation and/or expansion, such as by a method provided in Section II-D. In particular embodiments, one or more compositions of enriched T cells are engineered after the one or more compositions have been stimulated, activated, and/or incubated under stimulating conditions, such as described in methods provided in Section II-B. In particular embodiments, the one or more compositions are stimulated compositions. In particular embodiments, the one or more stimulated compositions have been previously cryofrozen and stored, and are thawed prior to engineering.

In certain embodiments, the one or more compositions of stimulated T cells are or include two separate stimulated compositions of enriched T cells. In particular embodiments, two separate compositions of enriched T cells, e.g., two separate compositions of enriched T cells that have been selected, isolated, and/or enriched from the same biological sample, are separately engineered. In certain embodiments, the two separate compositions include a composition of enriched CD4+ T cells. In particular embodiments, the two separate compositions include a composition of enriched CD8+ T cells. In some embodiments, two separate compositions of enriched CD4+ T cells and enriched CD8+ T cells, such as following incubation under stimulating conditions as described above, are genetically engineered separately. In some embodiments, a single composition of enriched T cells is genetically engineered. In certain embodiments, the single composition is a composition of enriched CD4+ T cells. In some embodiments, the single composition is a composition of enriched CD4+ and CD8+ T cells that have been combined from separate compositions prior to the engineering.

In some embodiments, the composition of enriched CD4+ T cells, such as stimulated CD4+ T cells, that is engineered, e.g., transduced or transfected, includes at least 60%, at least 65%, at least 70%, at least 75%, at least 80%, at least 85%, at least 90%, at least 95%, at least 98%, at least 99%, at least 99.5%, at least 99.9%, or at or at about 100% CD4+ T cells. In certain embodiments, the composition of enriched CD4+ T cells, such as stimulated CD4+ T cells, that is engineered includes less than 40%, less than 35%, less than 30%, less than 25%, less than 20%, less than 15%, less than 10%, less than 5%, less than 1%, less than 0.1%, or less than 0.01% CD8+ T cells, and/or contains no CD8+ T cells, and/or is free or substantially free of CD8+ T cells.

In some embodiments, the composition of enriched CD8+ T cells, such as stimulated CD8+ T cells, that is engineered, e.g., transduced or transfected, includes at least 60%, at least 65%, at least 70%, at least 75%, at least 80%, at least 85%, at least 90%, at least 95%, at least 98%, at least 99%, at least 99.5%, at least 99.9%, or at or at about 100% CD8+ T cells. In certain embodiments, the composition of enriched CD8+ T cells that, such as stimulated CD8+ T cells, that is engineered includes less than 40%, less than 35%, less than 30%, less than 25%, less than 20%, less than 15%, less than 10%, less than 5%, less than 1%, less than 0.1%, or less than 0.01% CD4+ T cells, and/or contains no CD4+ T cells, and/or is free or substantially free of CD4+ T cells.

In some embodiments, separate compositions of enriched CD4+ and CD8+ T cells are combined into a single composition and are genetically engineered, e.g., transduced or transfected. In certain embodiments, separate engineered compositions of enriched CD4+ and enriched CD8+ T cells are combined into a single composition after the genetic engineering has been performed and/or completed. In particular embodiments, separate compositions of enriched CD4+ and CD8+ T cells, such as separate compositions of stimulated CD4+ and CD8+ T cells are separately engineered and are separately processed for cultivation and/or expansion of T cells after the genetic engineering and been performed and/or completed.

In some embodiments, the introduction of a polynucleotide, e.g., a recombinant polynucleotide encoding a recombinant protein, is carried out by contacting enriched CD4+ or CD8+ T cells, such as stimulated CD4+ or CD8+ T cells, with a viral particles containing the polynucleotide. In some embodiments, contacting can be effected with centrifugation, such as spinoculation (e.g., centrifugal inoculation). In some embodiments, the composition containing cells, viral particles and reagent can be rotated, generally at relatively low force or speed, such as speed lower than that used to pellet the cells, such as from 600 rpm to 1700 rpm or from about 600 rpm to about 1700 rpm (e.g., at or about or at least 600 rpm, 1000 rpm, or 1500 rpm or 1700 rpm). In some embodiments, the rotation is carried at a force, e.g., a relative centrifugal force, of from 100 g to 3200 g or from about 100 g to about 3200 g (e.g., at or about or at least at or about 100 g, 200 g, 300 g, 400 g, 500 g, 1000 g, 1500 g, 2000 g, 2500 g, 3000 g or 3200 g), such as at or about 693 g, as measured for example at an internal or external wall of the chamber or cavity. The term “relative centrifugal force” or RCF is generally understood to be the effective force imparted on an object or substance (such as a cell, sample, or pellet and/or a point in the chamber or other container being rotated), relative to the earth's gravitational force, at a particular point in space as compared to the axis of rotation. The value may be determined using well-known formulas, taking into account the gravitational force, rotation speed and the radius of rotation (distance from the axis of rotation and the object, substance, or particle at which RCF is being measured). In some embodiments, at least a portion of the contacting, incubating, and/or engineering of the cells, e.g., cells from an stimulated composition of enriched CD4+ T cell or enriched CD8+ T cells, with the virus is performed with a rotation of between about 100 g and 3200 g, 1000 g and 2000 g, 1000 g and 3200 g, 500 g and 1000 g, 400 g and 1200 g, 600 g and 800 g, 600 and 700 g, or 500 g and 700 g. In some embodiments, the rotation is between 600 g and 700 g, e.g., at or about 693 g.

In certain embodiments, at least a portion of the engineering, transduction, and/or transfection is performed with rotation, e.g., spinoculation and/or centrifugation. In some embodiments, the rotation is performed for, for about, or for at least or about 5 minutes, 10 minutes, 15 minutes, 30 minutes, 60 minutes, 90 minutes, 1 hour, 2 hours, 3 hours, 4 hours, 6 hours, 8 hours, 12 hours, 24 hours, 48 hours, 72 hours, 2 days, 3 days, 4 days, 5 days, 6 days, or for at least 7 days. In some embodiments, the rotation is performed for or for about 60 minutes. In certain embodiments, the rotation is performed for about 30 minutes. In some embodiments, the rotation performed for about 30 minutes at between 600 g and 700 g, e.g., at or about 693 g.

In certain embodiments, the number of viable cells to be engineered, transduced, and/or transfected ranges from about 5×10⁶ cells to about 100×10⁷ cells, such as from about 10×10⁶ cells to about 100×10⁶ cells, from about 100×10⁶ cells to about 200×10⁶ cells, from about 200×10⁶ cells to about 300×10⁶ cells, from about 300×10⁶ cells to about 400×10⁶ cells, from about 400×10⁶ cells to about 500×10⁶ cells, or from about 500×10⁶ cells to about 100×10⁷ cells. In particular examples, the number of viable cells to be engineered, transduced, and/or transfected is about or less than about 300×10⁶ cells.

In certain embodiments, at least a portion of the engineering, transduction, and/or transfection is conducted at a volume (e.g., the spinoculation volume) from about 5 mL to about 100 mL, such as from about 10 mL to about 50 mL, from about 15 mL to about 45 mL, from about 20 mL to about 40 mL, from about 25 mL to about 35 mL, or at or at about 30 mL. In certain embodiments, the cell pellet volume after spinoculation ranges from about 1 mL to about 25 mL, such as from about 5 mL to about 20 mL, from about 5 mL to about 15 mL, from about 5 mL to about 10 mL, or at or at about 10 mL.

In some embodiments, gene transfer is accomplished by first stimulating the cell, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications. In certain embodiments, the gene transfer is accomplished by first incubating the cells under stimulating conditions, such as by any of the methods described in Section II-B.

In some embodiments, methods for genetic engineering are carried out by contacting one or more cells of a composition with a nucleic acid molecule encoding the recombinant protein, e.g., recombinant receptor. In some embodiments, the contacting can be effected with centrifugation, such as spinoculation (e.g., centrifugal inoculation). Such methods include any of those as described in International Publication Number WO2016/073602. Exemplary centrifugal chambers include those produced and sold by Biosafe SA, including those for use with the Sepax® and Sepax® 2 system, including an A-200/F and A-200 centrifugal chambers and various kits for use with such systems. Exemplary chambers, systems, and processing instrumentation and cabinets are described, for example, in U.S. Pat. Nos. 6,123,655, 6,733,433 and Published U.S. Patent Application, Publication No.: US 2008/0171951, and published international patent application, publication no. WO 00/38762, the contents of each of which are incorporated herein by reference in their entirety. Exemplary kits for use with such systems include, but are not limited to, single-use kits sold by BioSafe SA under product names CS-430.1, CS-490.1, CS-600.1 or CS-900.2.

In some embodiments, the system is included with and/or placed into association with other instrumentation, including instrumentation to operate, automate, control and/or monitor aspects of the transduction step and one or more various other processing steps performed in the system, e.g., one or more processing steps that can be carried out with or in connection with the centrifugal chamber system as described herein or in International Publication Number WO2016/073602. This instrumentation in some embodiments is contained within a cabinet. In some embodiments, the instrumentation includes a cabinet, which includes a housing containing control circuitry, a centrifuge, a cover, motors, pumps, sensors, displays, and a user interface. An exemplary device is described in U.S. Pat. Nos. 6,123,655, 6,733,433 and US 2008/0171951.

In some embodiments, the system comprises a series of containers, e.g., bags, tubing, stopcocks, clamps, connectors, and a centrifuge chamber. In some embodiments, the containers, such as bags, include one or more containers, such as bags, containing the cells to be transduced and the viral vector particles, in the same container or separate containers, such as the same bag or separate bags. In some embodiments, the system further includes one or more containers, such as bags, containing medium, such as diluent and/or wash solution, which is pulled into the chamber and/or other components to dilute, resuspend, and/or wash components and/or compositions during the methods. The containers can be connected at one or more positions in the system, such as at a position corresponding to an input line, diluent line, wash line, waste line and/or output line.

In some embodiments, the chamber is associated with a centrifuge, which is capable of effecting rotation of the chamber, such as around its axis of rotation. Rotation may occur before, during, and/or after the incubation in connection with transduction of the cells and/or in one or more of the other processing steps. Thus, in some embodiments, one or more of the various processing steps is carried out under rotation, e.g., at a particular force. The chamber is typically capable of vertical or generally vertical rotation, such that the chamber sits vertically during centrifugation and the side wall and axis are vertical or generally vertical, with the end wall(s) horizontal or generally horizontal.

In some embodiments, the composition containing cells and composition containing viral vector particles, and optionally air, can be combined or mixed prior to providing the compositions to the cavity. In some embodiments, the composition containing cells and composition containing viral vector particles, and optionally air, are provided separately and combined and mixed in the cavity. In some embodiments, a composition containing cells, a composition containing viral vector particles, and optionally air, can be provided to the internal cavity in any order. In any of such some embodiments, a composition containing cells and viral vector particles is the input composition once combined or mixed together, whether such is combined or mixed inside or outside the centrifugal chamber and/or whether cells and viral vector particles are provided to the centrifugal chamber together or separately, such as simultaneously or sequentially.

In some embodiments, intake of a volume of gas, such as air, occurs prior to the incubating the cells and viral vector particles, such as rotation, in the transduction method. In some embodiments, intake of the volume of gas, such as air, occurs during the incubation of the cells and viral vector particles, such as rotation, in the transduction method.

In some embodiments, the liquid volume of the cells or viral vector particles that make up the transduction composition, and optionally the volume of air, can be a predetermined volume. The volume can be a volume that is programmed into and/or controlled by circuitry associated with the system.

In some embodiments, intake of the transduction composition, and optionally gas, such as air, is controlled manually, semi-automatically and/or automatically until a desired or predetermined volume has been taken into the internal cavity of the chamber. In some embodiments, a sensor associated with the system can detect liquid and/or gas flowing to and from the centrifuge chamber, such as via its color, flow rate and/or density, and can communicate with associated circuitry to stop or continue the intake as necessary until intake of such desired or predetermined volume has been achieved. In some aspects, a sensor that is programmed or able only to detect liquid in the system, but not gas (e.g., air), can be made able to permit passage of gas, such as air, into the system without stopping intake. In some such embodiments, a non-clear piece of tubing can be placed in the line near the sensor while intake of gas, such as air, is desired. In some embodiments, intake of gas, such as air, can be controlled manually.

In aspects of the provided methods, the internal cavity of the centrifuge chamber is subjected to high speed rotation. In some embodiments, rotation is effected prior to, simultaneously, subsequently or intermittently with intake of the liquid input composition, and optionally air. In some embodiments, rotation is effected subsequent to intake of the liquid input composition, and optionally air. In some embodiments, rotation is by centrifugation of the centrifugal chamber at a relative centrifugal force at the inner surface of side wall of the internal cavity and/or at a surface layer of the cells of at or about or at least at or about 800 g, 1000 g, 1100 g, 1500, 1600 g, 1800 g, 2000 g, 2200 g, 2500 g, 3000 g, 3500 g or 4000 g. In some embodiments, rotation is by centrifugation at a force that is greater than or about 1100 g, such as by greater than or about 1200 g, greater than or about 1400 g, greater than or about 1600 g, greater than or about 1800 g, greater than or about 2000 g, greater than or about 2400 g, greater than or about 2800 g, greater than or about 3000 g or greater than or about 3200 g. In some embodiments, rotation is by centrifugation at a force that is or is about 1600 g.

In some embodiments, the method of transduction includes rotation or centrifugation of the transduction composition, and optionally air, in the centrifugal chamber for greater than or about 5 minutes, such as greater than or about 10 minutes, greater than or about 15 minutes, greater than or about 20 minutes, greater than or about 30 minutes, greater than or about 45 minutes, greater than or about 60 minutes, greater than or about 90 minutes or greater than or about 120 minutes. In some embodiments, the transduction composition, and optionally air, is rotated or centrifuged in the centrifugal chamber for greater than 5 minutes, but for no more than 60 minutes, no more than 45 minutes, no more than 30 minutes or no more than 15 minutes. In particular embodiments, the transduction includes rotation or centrifugation for or for about 60 minutes.

In some embodiments, the method of transduction includes rotation or centrifugation of the transduction composition, and optionally air, in the centrifugal chamber for between or between about 10 minutes and 60 minutes, 15 minutes and 60 minutes, 15 minutes and 45 minutes, 30 minutes and 60 minutes or 45 minutes and 60 minutes, each inclusive, and at a force at the internal surface of the side wall of the internal cavity and/or at a surface layer of the cells of at least or greater than or about 1000 g, 1100 g, 1200 g, 1400 g, 1500 g, 1600 g, 1800 g, 2000 g, 2200 g, 2400 g, 2800 g, 3200 g or 3600 g. In particular embodiments, the method of transduction includes rotation or centrifugation of the transduction composition, e.g., the cells and the viral vector particles, at or at about 1600 g for or for about 60 minutes.

In some embodiments, the gas, such as air, in the cavity of the chamber is expelled from the chamber. In some embodiments, the gas, such as air, is expelled to a container that is operably linked as part of the closed system with the centrifugal chamber. In some embodiments, the container is a free or empty container. In some embodiments, the air, such as gas, in the cavity of the chamber is expelled through a filter that is operably connected to the internal cavity of the chamber via a sterile tubing line. In some embodiments, the air is expelled using manual, semi-automatic or automatic processes. In some embodiments, air is expelled from the chamber prior to, simultaneously, intermittently or subsequently with expressing the output composition containing incubated cells and viral vector particles, such as cells in which transduction has been initiated or cells have been transduced with a viral vector, from the cavity of the chamber.

In some embodiments, the transduction and/or other incubation is performed as or as part of a continuous or semi-continuous process. In some embodiments, a continuous process involves the continuous intake of the cells and viral vector particles, e.g., the transduction composition (either as a single pre-existing composition or by continuously pulling into the same vessel, e.g., cavity, and thereby mixing, its parts), and/or the continuous expression or expulsion of liquid, and optionally expelling of gas (e.g., air), from the vessel, during at least a portion of the incubation, e.g., while centrifuging. In some embodiments, the continuous intake and continuous expression are carried out at least in part simultaneously. In some embodiments, the continuous intake occurs during part of the incubation, e.g., during part of the centrifugation, and the continuous expression occurs during a separate part of the incubation. The two may alternate. Thus, the continuous intake and expression, while carrying out the incubation, can allow for a greater overall volume of sample to be processed, e.g., transduced.

In some embodiments, the incubation is part of a continuous process, the method including, during at least a portion of the incubation, effecting continuous intake of said transduction composition into the cavity during rotation of the chamber and during a portion of the incubation, effecting continuous expression of liquid and, optionally expelling of gas (e.g., air), from the cavity through the at least one opening during rotation of the chamber.

In some embodiments, the semi-continuous incubation is carried out by alternating between effecting intake of the composition into the cavity, incubation, expression of liquid from the cavity and, optionally expelling of gas (e.g., air) from the cavity, such as to an output container, and then intake of a subsequent (e.g., second, third, etc.) composition containing more cells and other reagents for processing, e.g., viral vector particles, and repeating the process. For example, in some embodiments, the incubation is part of a semi-continuous process, the method including, prior to the incubation, effecting intake of the transduction composition into the cavity through said at least one opening, and subsequent to the incubation, effecting expression of fluid from the cavity; effecting intake of another transduction composition comprising cells and the viral vector particles into said internal cavity; and incubating the another transduction composition in said internal cavity under conditions whereby said cells in said another transduction composition are transduced with said vector. The process may be continued in an iterative fashion for a number of additional rounds. In this respect, the semi-continuous or continuous methods may permit production of even greater volume and/or number of cells.

In some embodiments, a portion of the transduction incubation is performed in the centrifugal chamber, which is performed under conditions that include rotation or centrifugation.

In some embodiments, the method includes an incubation in which a further portion of the incubation of the cells and viral vector particles is carried out without rotation or centrifugation, which generally is carried out subsequent to the at least portion of the incubation that includes rotation or centrifugation of the chamber. In certain embodiments, the incubation of the cells and viral vector particles is carried out without rotation or centrifugation for at least 1 hour, 6 hours, 12 hours, 24 hours, 32 hours, 48 hours, 60 hours, 72 hours, 90 hours, 96 hours, 3 days, 4 days, 5 days, or greater than 5 days. In certain embodiments, the incubation is carried out for or for about 72 hours.

In some such embodiments, the further incubation is effected under conditions to result in integration of the viral vector into a host genome of one or more of the cells. It is within the level of a skilled artisan to assess or determine if the incubation has resulted in integration of viral vector particles into a host genome, and hence to empirically determine the conditions for a further incubation. In some embodiments, integration of a viral vector into a host genome can be assessed by measuring the level of expression of a recombinant protein, such as a heterologous protein, encoded by a nucleic acid contained in the genome of the viral vector particle following incubation. A number of well-known methods for assessing expression level of recombinant molecules may be used, such as detection by affinity-based methods, e.g., immunoaffinity-based methods, e.g., in the context of cell surface proteins, such as by flow cytometry. In some examples, the expression is measured by detection of a transduction marker and/or reporter construct. In some embodiments, nucleic acid encoding a truncated surface protein is included within the vector and used as a marker of expression and/or enhancement thereof.

In some embodiments, the composition containing cells, the vector, e.g., viral particles, and reagent can be rotated, generally at relatively low force or speed, such as speed lower than that used to pellet the cells, such as from 600 rpm to 1700 rpm or from about 600 rpm to about 1700 rpm (e.g., at or about or at least 600 rpm, 1000 rpm, or 1500 rpm or 1700 rpm). In some embodiments, the rotation is carried at a force, e.g., a relative centrifugal force, of from 100 g to 3200 g or from about 100 g to about 3200 g (e.g., at or about or at least at or about 100 g, 200 g, 300 g, 400 g, 500 g, 1000 g, 1500 g, 2000 g, 2500 g, 3000 g or 3200 g), as measured for example at an internal or external wall of the chamber or cavity. The term “relative centrifugal force” or RCF is generally understood to be the effective force imparted on an object or substance (such as a cell, sample, or pellet and/or a point in the chamber or other container being rotated), relative to the earth's gravitational force, at a particular point in space as compared to the axis of rotation. The value may be determined using well-known formulas, taking into account the gravitational force, rotation speed and the radius of rotation (distance from the axis of rotation and the object, substance, or particle at which RCF is being measured).

In some embodiments, during at least a part of the genetic engineering, e.g., transduction, and/or subsequent to the genetic engineering the cells are transferred to the bioreactor bag assembly for culture of the genetically engineered cells, such as for cultivation or expansion of the cells, as described above.

In certain embodiments, a composition of enriched T cells in engineered, e.g., transduced or transfected, in the presence of a transduction adjuvant. In some embodiments, a composition of enriched T cells is engineered in the presence of one or more polycations. In some embodiments, a composition of enriched T cells is transduced, e.g., incubated with a viral vector particle, in the presence of one or more transduction adjuvants. In particular embodiments, a composition of enriched T cells is transfected, e.g., incubated with a non-viral vector, in the presence of one or more transduction adjuvants. In certain embodiments, the presence of one or more transduction adjuvants increases the efficiency of gene delivery, such as by increasing the amount, portion, and/or percentage of cells of the composition that are engineered (e.g., transduced or transfected). In certain embodiments, the presence of one or more transduction adjuvants increases the efficiency of transfection. In certain embodiments, the presence of one or more transduction adjuvants increases the efficiency of transduction. In particular embodiments, at least 25%, at least 30%, at least 40%, at least 50%, at least 60%, at least 70% at least 75%, at least 80%, at least 85%, at least 90%, at least 95%, or at least 99% of the cells that are engineered in the presence of a polycation contain or express the recombinant polynucleotide. In some embodiments, at least 10%, at least 20%, at least 30%, at least 40%, at least 50%, at least 60%, at least 70%, at least 80%, at least 90%, at least 95%, at least 100%, at least 150%, at least 1-fold, at least 2-fold, at least 3-fold, at least 4-fold, at least 5-fold, at least 10-fold, at least 25-Fold, at least 50-fold, or at least 100-fold more cells of a composition are engineered to contain or express the recombinant transduction adjuvants in the presence of a polycation as compared to an alternative and/or exemplary method of engineering cells without the presence of a transduction adjuvant.

In some embodiments, the composition of enriched cells are engineered in the presence of less than 100 μg/ml, less than 90 μg/ml, less than 80 μg/ml, less than 75 μg/ml, less than 70 μg/ml, less than 60 μg/ml, less than 50 μg/ml, less than 40 μg/ml, less than 30 μg/ml, less than 25 μg/ml, less than 20 μg/ml, or less than μg/ml, less than 10 μg/ml of a transduction adjuvant. In certain embodiments, transduction adjuvants suitable for use with the provided methods include, but are not limited to polycations, fibronectin or fibronectin-derived fragments or variants, RetroNectin, and combinations thereof.

In some embodiments, the cells are engineered in the presence of a cytokine, e.g., a recombinant human cytokine, at a concentration of between 1 IU/ml and 1,000 IU/ml, between 10 IU/ml and 50 IU/ml, between 50 IU/ml and 100 IU/ml, between 100 IU/ml and 200 IU/ml, between 100 IU/ml and 500 IU/ml, between 250 IU/ml and 500 IU/ml, or between 500 IU/ml and 1,000 IU/ml.

In some embodiments, a composition of enriched T cells is engineered in the presence of IL-2, e.g., human recombinant IL-2, at a concentration between 1 IU/ml and 200 IU/ml, between 10 IU/ml and 100 IU/ml, between 50 IU/ml and 150 IU/ml, between 80 IU/ml and 120 IU/ml, between 60 IU/ml and 90 IU/ml, or between 70 IU/ml and 90 IU/ml. In particular embodiments, the composition of enriched T cells is engineered in the presence of recombinant IL-2 at a concentration at or at about 50 IU/ml, 55 IU/ml, 60 IU/ml, 65 IU/ml, 70 IU/ml, 75 IU/ml, 80 IU/ml, 85 IU/ml, 90 IU/ml, 95 IU/ml, 100 IU/ml, 110 IU/ml, 120 IU/ml, 130 IU/ml, 140 IU/ml, or 150 IU/ml. In some embodiments, the composition of enriched T cells is engineered in the presence of or of about 85 IU/ml. In some embodiments, the population of T cells is a population of CD4+ T cells. In particular embodiments, the composition of enriched T cells is enriched for CD4+ T cells, where CD8+ T cells are not enriched for and/or where CD8+ T cells are negatively selected for or depleted from the composition. In particular embodiments, the composition of enriched T cells is a composition of enriched CD8+ T cells. In particular embodiments, the composition of enriched T cells is enriched for CD8+ T cells, where CD4+ T cells are not enriched for and/or where CD4+ T cells are negatively selected for or depleted from the composition.

In some embodiments, a composition of enriched T cells is engineered in the presence of recombinant IL-7, e.g., human recombinant IL-7, at a concentration between 100 IU/ml and 2,000 IU/ml, between 500 IU/ml and 1,000 IU/ml, between 100 IU/ml and 500 IU/ml, between 500 IU/ml and 750 IU/ml, between 750 IU/ml and 1,000 IU/ml, or between 550 IU/ml and 650 IU/ml. In particular embodiments, the composition of enriched T cells is engineered in the presence of IL-7 at a concentration at or at about 50 IU/ml, 100 IU/ml, 150 IU/ml, 200 IU/ml, 250 IU/ml, 300 IU/ml, 350 IU/ml, 400 IU/ml, 450 IU/ml, 500 IU/ml, 550 IU/ml, 600 IU/ml, 650 IU/ml, 700 IU/ml, 750 IU/ml, 800 IU/ml, 750 IU/ml, 750 IU/ml, 750 IU/ml, or 1,000 IU/ml. In particular embodiments, the composition of enriched T cells is engineered in the presence of or of about 600 IU/ml of IL-7. In some embodiments, the composition engineered in the presence of recombinant IL-7 is enriched for a population of T cells, e.g., CD4+ T cells. In particular embodiments, the composition of enriched T cells is enriched for CD4+ T cells, where CD8+ T cells are not enriched for and/or where CD8+ T cells are negatively selected for or depleted from the composition.

In some embodiments, a composition of enriched T cells is engineered in the presence of recombinant IL-15, e.g., human recombinant IL-15, at a concentration between 0.1 IU/ml and 100 IU/ml, between 1 IU/ml and 50 IU/ml, between 5 IU/ml and 25 IU/ml, between 25 IU/ml and 50 IU/ml, between 5 IU/ml and 15 IU/ml, or between 10 IU/ml and 100 IU/ml. In particular embodiments, the composition of enriched T cells is engineered in the presence of IL-15 at a concentration at or at about 1 IU/ml, 2 IU/ml, 3 IU/ml, 4 IU/ml, 5 IU/ml, 6 IU/ml, 7 IU/ml, 8 IU/ml, 9 IU/ml, 10 IU/ml, 11 IU/ml, 12 IU/ml, 13 IU/ml, 14 IU/ml, 15 IU/ml, 20 IU/ml, 25 IU/ml, 30 IU/ml, 40 IU/ml, or 50 IU/ml. In some embodiments, the composition of enriched T cells is engineered in or in about 10 IU/ml of IL-15. In some embodiments, the composition of enriched T cells is incubated in or in about 10 IU/ml of recombinant IL-15. In some embodiments, the composition engineered in the presence of recombinant IL-15 is enriched for a population of T cells, e.g., CD4+ T cells and/or CD8+ T cells. In some embodiments, the composition of enriched T cells is a composition of enriched CD8+ T cells. In particular embodiments, the composition of enriched T cells is enriched for CD8+ T cells, where CD4+ T cells are not enriched for and/or where CD4+ T cells are negatively selected for or depleted from the composition. In some embodiments, the composition of enriched T cells is a composition of enriched CD4+ T cells. In particular embodiments, the composition of enriched T cells is enriched for CD4+ T cells, where CD8+ T cells are not enriched for and/or where CD8+ T cells are negatively selected for or depleted from the composition.

In particular embodiments, a composition of enriched CD8+ T cells is engineered in the presence of IL-2 and/or IL-15. In certain embodiments, a composition of enriched CD4+ T cells is engineered in the presence of IL-2, IL-7, and/or IL-15. In some embodiments, the IL-2, IL-7, and/or IL-15 are recombinant. In certain embodiments, the IL-2, IL-7, and/or IL-15 are human. In particular embodiments, the one or more cytokines are or include human recombinant IL-2, IL-7, and/or IL-15.

In particular embodiments, the cells are engineered in the presence of one or more antioxidants. In some embodiments, antioxidants include, but are not limited to, one or more antioxidants comprise a tocopherol, a tocotrienol, alpha-tocopherol, beta-tocopherol, gamma-tocopherol, delta-tocopherol, alpha-tocotrienol, beta-tocotrienol, alpha-tocopherolquinone, Trolox (6-hydroxy-2,5,7,8-tetramethylchroman-2-carboxylic acid), butylated hydroxyanisole (BHA), butylated hydroxytoluene (BHT), a flavonoids, an isoflavone, lycopene, beta-carotene, selenium, ubiquinone, luetin, S-adenosylmethionine, glutathione, taurine, N-acetyl cysteine (NAC), citric acid, L-carnitine, BHT, monothioglycerol, ascorbic acid, propyl gallate, methionine, cysteine, homocysteine, gluthatione, cystamine and cystathionine, and/or glycine-glycine-histidine.

In some embodiments, the one or more antioxidants is or includes a sulfur containing oxidant. In certain embodiments, a sulfur containing antioxidant may include thiol-containing antioxidants and/or antioxidants which exhibit one or more sulfur moieties, e.g., within a ring structure. In some embodiments, the sulfur containing antioxidants may include, for example, N-acetyl cysteine (NAC) and 2,3-dimercaptopropanol (DMP), L-2-oxo-4-thiazolidinecarboxylate (OTC) and lipoic acid. In particular embodiments, the sulfur containing antioxidant is a glutathione precursor. In some embodiments, the glutathione precursor is a molecule which may be modified in one or more steps within a cell to derived glutathione. In particular embodiments, a glutathione precursor may include, but is not limited to N-acetyl cysteine (NAC), L-2-oxothiazolidine-4-carboxylic acid (Procysteine), lipoic acid, S-allyl cysteine, or methylmethionine sulfonium chloride.

In some embodiments, the cells are engineered in the presence of one or more antioxidants. In some embodiments, the cells are engineered in the presence of between 1 ng/ml and 100 ng/ml, between 10 ng/ml and 1 μg/ml, between 100 ng/ml and 10 μg/ml, between 1 μg/ml and 100 μg/ml, between 10 μg/ml and 1 mg/ml, between 100 μg/ml and 1 mg/ml, between 1 500 μg/ml and 2 mg/ml, 500 μg/ml and 5 mg/ml, between 1 mg/ml and 10 mg/ml, or between 1 mg/ml and 100 mg/ml of the one or more antioxidants. In some embodiments, the cells are engineered in the presence of or of about 1 ng/ml, 10 ng/ml, 100 ng/ml, 1 μg/ml, 10 μg/ml, 100 μg/ml, 0.2 mg/ml, 0.4 mg/ml, 0.6 mg/ml, 0.8 mg/ml, 1 mg/ml, 2 mg/ml, 3 mg/ml, 4 mg/ml, 5 mg/ml, 10 mg/ml, 20 mg/ml, 25 mg/ml, 50 mg/ml, 100 mg/ml, 200 mg/ml, 300 mg/ml, 400 mg/ml, 500 mg/ml of the one or more antioxidant. In some embodiments, the one or more antioxidants is or includes a sulfur containing antioxidant. In particular embodiments, the one or more antioxidants is or includes a glutathione precursor.

In some embodiments, the cells are engineered in the presence of NAC. In some embodiments, the cells are engineered in the presence of between 1 ng/ml and 100 ng/ml, between 10 ng/ml and 1 μg/ml, between 100 ng/ml and 10 μg/ml, between 1 μg/ml and 100 μg/ml, between 10 μg/ml and 1 mg/ml, between 100 μg/ml and 1 mg/ml, between 1,500 μg/ml and 2 mg/ml, 500 μg/ml and 5 mg/ml, between 1 mg/ml and 10 mg/ml, or between 1 mg/ml and 100 mg/ml of NAC. In some embodiments, the cells are engineered in the presence of or of about 1 ng/ml, 10 ng/ml, 100 ng/ml, 1 μg/ml, 10 μg/ml, 100 μg/ml, 0.2 mg/ml, 0.4 mg/ml, 0.6 mg/ml, 0.8 mg/ml, 1 mg/ml, 2 mg/ml, 3 mg/ml, 4 mg/ml, 5 mg/ml, 10 mg/ml, 20 mg/ml, 25 mg/ml, 50 mg/ml, 100 mg/ml, 200 mg/ml, 300 mg/ml, 400 mg/ml, 500 mg/ml of NAC. In some embodiments, the cells are engineered with or with about 0.8 mg/ml.

In some embodiments, a composition of enriched T cells, such as stimulated T cells, e.g., stimulated CD4+ T cells or stimulated CD8+ T cells, is engineered in the presence of one or more polycations. In some embodiments, a composition of enriched T cells, such as stimulated T cells, e.g., stimulated CD4+ T cells or stimulated CD8+ T cells, is transduced, e.g., incubated with a viral vector particle, in the presence of one or more polycations. In particular embodiments, a composition of enriched T cells, such as stimulated T cells, e.g., stimulated CD4+ T cells or stimulated CD8+ T cells, is transfected, e.g., incubated with a non-viral vector, in the presence of one or more polycations. In certain embodiments, the presence of one or more polycations increases the efficiency of gene delivery, such as by increasing the amount, portion, and/or percentage of cells of the composition that are engineered (e.g., transduced or transfected). In certain embodiments, the presence of one or more polycations increases the efficiency of transfection. In certain embodiments, the presence of one or more polycations increases the efficiency of transduction. In particular embodiments, at least 25%, at least 30%, at least 40%, at least 50%, at least 60%, at least 70% at least 75%, at least 80%, at least 85%, at least 90%, at least 95%, or at least 99% of the cells that are engineered in the presence of a polycation contain or express the recombinant polynucleotide. In some embodiments, at least 10%, at least 20%, at least 30%, at least 40%, at least 50%, at least 60%, at least 70%, at least 80%, at least 90%, at least 95%, at least 100%, at least 150%, at least 1-fold, at least 2-fold, at least 3-fold, at least 4-fold, at least 5-fold, at least 10-fold, at least 25-Fold, at least 50-fold, or at least 100-fold more cells of a composition are engineered to contain or express the recombinant polynucleotide in the presence of a polycation as compared to an alternative and/or exemplary method of engineering cells without the presence of a polycation.

In certain embodiments, the composition of enriched cells, e.g., the composition of enriched CD4+ T cells or enriched CD8+ T cells, such as stimulated T cells thereof, is engineered in the presence of a low concentration or amount of a polycation, e.g., relative to an exemplary and/or alternative method of engineering cells in the presence of a polycation. In certain embodiments, the composition of enriched cells, such as stimulated T cells, e.g., stimulated CD4+ T cells or stimulated CD8+ T cells, is engineered in the presence of less than 90%, less than 80%, less than 75%, less than 70%, less than 60%, less than 50%, less than 40%, less than 30%, less than 25%, less than 20%, less than 10%, less than 5%, less than 1%, less than 0.1%, of less than 0.01% of the amount and/or concentration of the polycation of an exemplary and/or alternative process for engineering cells. In some embodiments, the composition of enriched cells, such as stimulated T cells, e.g., stimulated CD4+ T cells or stimulated CD8+ T cells, are engineered in the presence of less than 100 μg/ml, less than 90 μg/ml, less than 80 μg/ml, less than 75 μg/ml, less than 70 μg/ml, less than 60 μg/ml, less than 50 μg/ml, less than 40 μg/ml, less than 30 μg/ml, less than 25 μg/ml, less than 20 μg/ml, or less than μg/ml, less than 10 μg/ml of the polycation. In particular embodiments, the composition of enriched cells, such as stimulated T cells, e.g., stimulated CD4+ T cells or stimulated CD8+ T cells, is engineered in the presence of or of about 1 μg/ml, 5 μg/ml, 10 μg/ml, 15 μg/ml, 20 μg/ml, 25 μg/ml, 30 μg/ml, 35 μg/ml, 40 μg/ml, 45 μg/ml, or 50 μg/ml, of the polycation.

In particular embodiments, engineering the composition of enriched cells, such as stimulated T cells, e.g., stimulated CD4+ T cells or stimulated CD8+ T cells, in the presence of a polycation reduces the amount of cell death, e.g., by necrosis, programed cell death, or apoptosis. In some embodiments, the composition of enriched T cells, such as stimulated T cells, e.g., stimulated CD4+ T cells or stimulated CD8+ T cells, is engineered in the presence of a low amount of a polycation, e.g., less than 100 μg/ml, 50 μg/ml, or 10 μg/ml, and at least 50%, at least 60%, at least 70%, at least 75%, at least 80%, at least 85%, at least 90%, at least 95%, at least 99%, or at least 99.9% of the cells survive, e.g., do not undergo necrosis, programed cell death, or apoptosis, during or at least 1 day, 2 days, 3 days, 4 days, 5 days, 6 days, 7 days, or more than 7 days after the engineering step is complete. In some embodiments, the composition is engineered in the presence of a low concentration or amount of polycation as compared to the alternative and/or exemplary method of engineering cells in the presence of higher amount or concentration of polycation, e.g., more than 50 μg/ml, 100 μg/ml, 500 μg/ml, or 1,000 μg/ml, and the cells of the composition have at least 10%, at least 20%, at least 30%, at least 40%, at least 50%, at least 60%, at least 70%, at least 80%, at least 90%, at least 95%, at least 100%, at least 150%, at least 1-fold, at least 2-fold, at least 3-fold, at least 4-fold, at least 5-fold, at least 10-fold, at least 25-Fold, at least 50-fold, or at least 100-fold greater survival as compared to cells undergoing the exemplary and/or alternative process.

In some embodiments, the polycation is positively-charged. In certain embodiments, the polycation reduces repulsion forces between cells and vectors, e.g., viral or non-viral vectors, and mediates contact and/or binding of the vector to the cell surface. In some embodiments, the polycation is polybrene, DEAE-dextran, protamine sulfate, poly-L-lysine, or cationic liposomes.

In particular embodiments, the polycation is protamine sulfate. In some embodiments, the composition of enriched T cells, such as stimulated T cells, e.g., stimulated CD4+ T cells or stimulated CD8+ T cells, are engineered in the presence of less than or about 500 μg/ml, less than or about 400 μg/ml, less than or about 300 μg/ml, less than or about 200 μg/ml, less than or about 150 μg/ml, less than or about 100 μg/ml, less than or about 90 μg/ml, less than or about 80 μg/ml, less than or about 75 μg/ml, less than or about 70 μg/ml, less than or about 60 μg/ml, less than or about 50 μg/ml, less than or about 40 μg/ml, less than or about 30 μg/ml, less than or about 25 μg/ml, less than or about 20 μg/ml, or less than or about 15 μg/ml, or less than or about 10 μg/ml of protamine sulfate. In particular embodiments, the composition of enriched cells, such as stimulated T cells, e.g., stimulated CD4+ T cells or stimulated CD8+ T cells, is engineered in the presence of or of about 1 μg/ml, 5 μg/ml, 10 μg/ml, 15 μg/ml, 20 μg/ml, 25 μg/ml, 30 μg/ml, 35 μg/ml, 40 μg/ml, 45 μg/ml, 50 μg/ml, 55 μg/ml, 60 μg/ml, 75 μg/ml, 80 μg/ml, 85 μg/ml, 90 μg/ml, 95 μg/ml, 100 μg/ml, 105 μg/ml, 110 μg/ml, 115 μg/ml, 120 μg/ml, 125 μg/ml, 130 μg/ml, 135 μg/ml, 140 μg/ml, 145 μg/ml, or 150 μg/ml of protamine sulfate.

In some embodiments, the engineered composition of enriched CD4+ T cells, such as stimulated T cells, e.g., stimulated CD4+ T cells, includes at least 40, at least 60%, at least 65%, at least 70%, at least 75%, at least 80%, at least 85%, at least 90%, at least 95%, at least 98%, at least 99%, at least 99.5%, at least 99.9%, or at or at about 100% CD4+ T cells. In certain embodiments, the composition of enriched CD4+ T cells, such as stimulated T cells, e.g., stimulated CD4+ T cells, that is engineered includes less than 40%, less than 35%, less than 30%, less than 25%, less than 20%, less than 15%, less than 10%, less than 5%, less than 1%, less than 0.1%, or less than 0.01% CD8+ T cells, and/or contains no CD8+ T cells, and/or is free or substantially free of CD8+ T cells.

In some embodiments, the composition of enriched CD8+ T cells, such as stimulated T cells, e.g., stimulated CD8+ T cells, that is engineered includes at least 60%, at least 65%, at least 70%, at least 75%, at least 80%, at least 85%, at least 90%, at least 95%, at least 98%, at least 99%, at least 99.5%, at least 99.9%, or at or at about 100% CD8+ T cells. In certain embodiments, the composition of enriched CD8+ T cells, such as stimulated T cells, e.g., stimulated CD8+ T cells, that is engineered includes less than 40%, less than 35%, less than 30%, less than 25%, less than 20%, less than 15%, less than 10%, less than 5%, less than 1%, less than 0.1%, or less than 0.01% CD4+ T cells, and/or contains no CD4+ T cells, and/or is free or substantially free of CD4+ T cells.

In some embodiments, engineering the cells includes a culturing, contacting, or incubation with the vector, e.g., the viral vector of the non-viral vector. In certain embodiments, the engineering includes culturing, contacting, and/or incubating the cells with the vector is performed for, for about, or for at least 4 hours, 6 hours, 8 hours, 12 hours, 16 hours, 18 hours, 24 hours, 30 hours, 36 hours, 40 hours, 48 hours, 54 hours, 60 hours, 72 hours, 84 hours, 1 day, 2 days, 3 days, 4 days, 5 days, 6 days, or 7 days, or more than 7 days. In particular embodiments, the engineering includes culturing, contacting, and/or incubating the cells with the vector for or for about 24 hours, 36 hours, 48 hours, 60 hours, 72 hours, or 84 hours, or for or for about 2 days, 3 days, 4 days, or 5 days. In some embodiments, the engineering step is performed for or for about 24 hours, 36 hours, 48 hours, 60 hours, 72 hours, or 84 hours. In certain embodiments, the engineering is performed for about 60 hours or about 84 hours, for or for about 72 hours, or for or for about 2 days.

In some embodiments, the engineering is performed at a temperature from about 25 to about 38° C., such as from about 30 to about 37° C., from about 36 to about 38° C., or at or about 37° C.±2° C. In some embodiments, the composition of enriched T cells is engineered at a CO₂ level from about 2.5% to about 7.5%, such as from about 4% to about 6%, for example at or about 5%±0.5%. In some embodiments, the composition of enriched T cells is engineered at a temperature of or about 37° C. and/or at a CO₂ level of or about 5%.

In some embodiments, the cells, e.g., the CD4+ and/or the CD8+ T cells, are cultivated, after one or more steps are performed for genetic engineering, e.g., transducing or transfection the cells to contain a polynucleotide encoding a recombinant receptor. In some embodiments, the cultivation may include culture, incubation, stimulation, activation, expansion, and/or propagation. In some such embodiments, the further cultivation is effected under conditions to result in integration of the viral vector into a host genome of one or more of the cells. The incubation and/or engineering may be carried out in a culture vessel, such as a unit, chamber, well, column, tube, tubing set, valve, vial, culture dish, bag, or other container for culture or cultivating cells. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor.

In some embodiments, the further incubation is carried out at temperatures greater than room temperature, such as greater than or greater than about 25° C., such as generally greater than or greater than about 32° C., 35° C. or 37° C. In some embodiments, the further incubation is effected at a temperature of at or about 37° C.±2° C., such as at a temperature of at or about 37° C.

In some embodiments, the further incubation is performed under conditions for stimulation and/or activation of cells, which conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent (e.g., stimulatory and/or accessory agents), e.g., ligand, which is capable of activating an intracellular signaling domain of a TCR complex. In some aspects, the agent turns on or initiates TCR/CD3 intracellular signaling cascade in a T cell, such as agents suitable to deliver a primary signal, e.g., to initiate activation of an ITAM-induced signal, such as those specific for a TCR component, and/or an agent that promotes a costimulatory signal, such as one specific for a T cell costimulatory receptor, e.g., anti-CD3, anti-CD28, or anti-41-BB, for example, optionally bound to solid support such as a bead, and/or one or more cytokines. Among the stimulating agents are anti-CD3/anti-CD28 beads (e.g., DYNABEADS® M-450 CD3/CD28 T Cell Expander, and/or ExpACT® beads). Optionally, the expansion method may further comprise the step of adding anti-CD3 and/or anti-CD28 antibody to the culture medium. In some embodiments, the stimulating agents include IL-2 and/or IL-15, for example, an IL-2 concentration of at least about 10 units/mL.

In some embodiments, the stimulating conditions or agents include one or more agent, e.g., ligand, which is capable of activating an intracellular signaling domain of a TCR complex. In some aspects, the agent turns on or initiates TCR/CD3 intracellular signaling cascade in a T cell. Such agents can include antibodies, such as those specific for a TCR component and/or costimulatory receptor, e.g., anti-CD3, anti-CD28, for example, bound to solid support such as a bead, and/or one or more cytokines. Optionally, the expansion method may further comprise the step of adding anti-CD3 and/or anti-CD28 antibody to the culture medium (e.g., at a concentration of at least about 0.5 ng/ml). In some embodiments, the stimulating agents include IL-2 and/or IL-15, for example, an IL-2 concentration of at least about 10 units/mL, at least about 50 units/mL, at least about 100 units/mL or at least about 200 units/mL.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some aspects, incubation is carried out in accordance with techniques such as those described in U.S. Pat. No. 6,040,177 to Riddell et al., Klebanoff et al. (2012) J Immunother. 35(9): 651-660, Terakura et al. (2012) Blood. 1:72-82, and/or Wang et al. (2012) J Immunother. 35(9):689-701.

In some embodiments, the further incubation is carried out in the same container or apparatus in which the contacting occurred. In some embodiments, the further incubation is carried out without rotation or centrifugation, which generally is carried out subsequent to the at least portion of the incubation done under rotation, e.g., in connection with centrifugation or spinoculation. In some embodiments, the further incubation is carried out outside of a stationary phase, such as outside of a chromatography matrix, for example, in solution.

In some embodiments, the further incubation is carried out in a different container or apparatus from that in which the contacting occurred, such as by transfer, e.g., automatic transfer, of the cell composition into a different container or apparatus subsequent to contacting with the viral particles and reagent.

In some embodiments, the further culturing or incubation, e.g., to facilitate ex vivo expansion, is carried out of for greater than or greater than about 24 hours, 2 days, 3 days, 4 days, 5 days, 6 days, 7 days, 8 days, 9 days, 10 days, 11 days, 12 days, 13 days or 14 days. In some embodiments, the further culturing or incubation is carried out for no more than 6 days, no more than 5 days, no more than 4 days, no more than 3 days, no more than 2 days or no more than 24 hours.

In some embodiments, the total duration of the incubation, e.g., with the stimulating agent, is between or between about 1 hour and 96 hours, 1 hour and 72 hours, 1 hour and 48 hours, 4 hours and 36 hours, 8 hours and 30 hours or 12 hours and 24 hours, such as at least or about at least or about 6 hours, 12 hours, 18 hours, 24 hours, 36 hours or 72 hours. In some embodiments, the further incubation is for a time between or about between 1 hour and 48 hours, 4 hours and 36 hours, 8 hours and 30 hours or 12 hours and 24 hours, inclusive.

In some embodiments, the methods provided herein do not include further culturing or incubation, e.g., do not include ex vivo expansion step, or include a substantially shorter ex vivo expansion step.

In some embodiments, the stimulatory reagent is removed and/or separated from the cells prior to the engineering. In particular embodiments, the stimulatory reagent is removed and/or separated from the cells after the engineering. In certain embodiments, the stimulatory agent is removed and/or separated from the cells subsequent to the engineering and prior to cultivating the engineered cells, e.g., under conditions that promote proliferation and/or expansion. In certain embodiments, the stimulatory reagent is a stimulatory reagent that is described in Section II-B-1. In particular embodiments, the stimulatory reagent is removed and/or separated from the cells as described in Section II-B-2.

### 1. Vectors and Methods

Also provided are one or more polynucleotides (e.g., nucleic acid molecules) encoding recombinant receptors, vectors for genetically engineering cells to express such receptors in accord with provided methods for producing the engineered cells. In some embodiments, the vector contains the nucleic acid encoding the recombinant receptor. In particular embodiments, the vector is a viral vector a non-viral vector. In some cases, the vector is a viral vector, such as a retroviral vector, e.g., a lentiviral vector or a gammaretroviral vector.

In some embodiments, the vectors include viral vectors, e.g., retroviral or lentiviral, non-viral vectors or transposons, e.g., Sleeping Beauty transposon system, vectors derived from simian virus 40 (SV40), adenoviruses, adeno-associated virus (AAV), lentiviral vectors or retroviral vectors, such as gamma-retroviral vectors, retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV) or adeno-associated virus (AAV).

In some embodiments, the viral vector or the non-viral DNA contains a nucleic acid that encodes a heterologous recombinant protein. In some embodiments, the heterologous recombinant molecule is or includes a recombinant receptor, e.g., an antigen receptor, SB-transposons, e.g., for gene silencing, capsid-enclosed transposons, homologous double stranded nucleic acid, e.g., for genomic recombination or reporter genes (e.g., fluorescent proteins, such as GFP) or luciferase).

a. Viral Vector Particles

In some embodiments, recombinant nucleic acids are transferred into cells using recombinant infectious virus particles, such as, e.g., vectors derived from simian virus 40 (SV40), adenoviruses, adeno-associated virus (AAV). In some embodiments, recombinant nucleic acids are transferred into T cells using recombinant lentiviral vectors or retroviral vectors, such as gamma-retroviral vectors (see, e.g., Koste et al. (2014) Gene Therapy 2014 Apr. 3. doi: 10.1038/gt.2014.25; Carlens et al. (2000) Exp Hematol 28(10): 1137-46; Alonso-Camino et al. (2013) Mol Ther Nucl Acids 2, e93; Park et al., Trends Biotechnol. 2011 Nov. 29(11): 550-557.

In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV), or adeno-associated virus (AAV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109.

Methods of lentiviral transduction are known. Exemplary methods are described in, e.g., Wang et al. (2012) J Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505.

In some embodiments, the viral vector particles contain a genome derived from a retroviral genome based vector, such as derived from a lentiviral genome based vector. In some aspects of the provided viral vectors, the heterologous nucleic acid encoding a recombinant receptor, such as an antigen receptor, such as a CAR, is contained and/or located between the 5′ LTR and 3′ LTR sequences of the vector genome.

In some embodiments, the viral vector genome is a lentivirus genome, such as an HIV-1 genome or an SIV genome. For example, lentiviral vectors have been generated by multiply attenuating virulence genes, for example, the genes env, vif, vpu and nef can be deleted, making the vector safer for therapeutic purposes. Lentiviral vectors are known. See Naldini et al., (1996 and 1998); Zufferey et al., (1997); Dull et al., 1998, U.S. Pat. Nos. 6,013,516; and 5,994,136). In some embodiments, these viral vectors are plasmid-based or virus-based, and are configured to carry the essential sequences for incorporating foreign nucleic acid, for selection, and for transfer of the nucleic acid into a host cell. Known lentiviruses can be readily obtained from depositories or collections such as the American Type Culture Collection (“ATCC”; 10801 University Blvd., Manassas, Va. 20110-2209), or isolated from known sources using commonly available techniques.

Non-limiting examples of lentiviral vectors include those derived from a lentivirus, such as Human Immunodeficiency Virus 1 (HIV-1), HIV-2, an Simian Immunodeficiency Virus (SIV), Human T-lymphotropic virus 1 (HTLV-1), HTLV-2 or equine infection anemia virus (E1AV). For example, lentiviral vectors have been generated by multiply attenuating the HIV virulence genes, for example, the genes env, vif, vpr, vpu and nef are deleted, making the vector safer for therapeutic purposes. Lentiviral vectors are known in the art, see Naldini et al., (1996 and 1998); Zufferey et al., (1997); Dull et al., 1998, U.S. Pat. Nos. 6,013,516; and 5,994,136). In some embodiments, these viral vectors are plasmid-based or virus-based, and are configured to carry the essential sequences for incorporating foreign nucleic acid, for selection, and for transfer of the nucleic acid into a host cell. Known lentiviruses can be readily obtained from depositories or collections such as the American Type Culture Collection (“ATCC”; 10801 University Blvd., Manassas, Va. 20110-2209), or isolated from known sources using commonly available techniques.

In some embodiments, the viral genome vector can contain sequences of the 5′ and 3′ LTRs of a retrovirus, such as a lentivirus. In some aspects, the viral genome construct may contain sequences from the 5′ and 3′ LTRs of a lentivirus, and in particular can contain the R and U5 sequences from the 5′ LTR of a lentivirus and an inactivated or self-inactivating 3′ LTR from a lentivirus. The LTR sequences can be LTR sequences from any lentivirus from any species. For example, they may be LTR sequences from HIV, SIV, FIV or BIV. Typically, the LTR sequences are HIV LTR sequences.

In some embodiments, the nucleic acid of a viral vector, such as an HIV viral vector, lacks additional transcriptional units. The vector genome can contain an inactivated or self-inactivating 3′ LTR (Zufferey et al. J Virol 72: 9873, 1998; Miyoshi et al., J Virol 72:8150, 1998). For example, deletion in the U3 region of the 3′ LTR of the nucleic acid used to produce the viral vector RNA can be used to generate self-inactivating (SIN) vectors. This deletion can then be transferred to the 5′ LTR of the proviral DNA during reverse transcription. A self-inactivating vector generally has a deletion of the enhancer and promoter sequences from the 3′ long terminal repeat (LTR), which is copied over into the 5′ LTR during vector integration. In some embodiments enough sequence can be eliminated, including the removal of a TATA box, to abolish the transcriptional activity of the LTR. This can prevent production of full-length vector RNA in transduced cells. In some aspects, the U3 element of the 3′ LTR contains a deletion of its enhancer sequence, the TATA box, Sp1, and NF-kappa B sites. As a result of the self-inactivating 3′ LTR, the provirus that is generated following entry and reverse transcription contains an inactivated 5′ LTR. This can improve safety by reducing the risk of mobilization of the vector genome and the influence of the LTR on nearby cellular promoters. The self-inactivating 3′ LTR can be constructed by any method known in the art. In some embodiments, this does not affect vector titers or the in vitro or in vivo properties of the vector.

Optionally, the U3 sequence from the lentiviral 5′ LTR can be replaced with a promoter sequence in the viral construct, such as a heterologous promoter sequence. This can increase the titer of virus recovered from the packaging cell line. An enhancer sequence can also be included. Any enhancer/promoter combination that increases expression of the viral RNA genome in the packaging cell line may be used. In one example, the CMV enhancer/promoter sequence is used (U.S. Pat. Nos. 5,385,839 and 5,168,062).

In certain embodiments, the risk of insertional mutagenesis can be minimized by constructing the retroviral vector genome, such as lentiviral vector genome, to be integration defective. A variety of approaches can be pursued to produce a non-integrating vector genome. In some embodiments, a mutation(s) can be engineered into the integrase enzyme component of the pol gene, such that it encodes a protein with an inactive integrase. In some embodiments, the vector genome itself can be modified to prevent integration by, for example, mutating or deleting one or both attachment sites, or making the 3′ LTR-proximal polypurine tract (PPT) non-functional through deletion or modification. In some embodiments, non-genetic approaches are available; these include pharmacological agents that inhibit one or more functions of integrase. The approaches are not mutually exclusive; that is, more than one of them can be used at a time. For example, both the integrase and attachment sites can be non-functional, or the integrase and PPT site can be non-functional, or the attachment sites and PPT site can be non-functional, or all of them can be non-functional. Such methods and viral vector genomes are known and available (see Philpott and Thrasher, Human Gene Therapy 18:483, 2007; Engelman et al. J Virol 69:2729, 1995; Brown et al J Virol 73:9011 (1999); WO 2009/076524; McWilliams et al., J Virol 77:11150, 2003; Powell and Levin J Virol 70:5288, 1996).

In some embodiments, the vector contains sequences for propagation in a host cell, such as a prokaryotic host cell. In some embodiments, the nucleic acid of the viral vector contains one or more origins of replication for propagation in a prokaryotic cell, such as a bacterial cell. In some embodiments, vectors that include a prokaryotic origin of replication also may contain a gene whose expression confers a detectable or selectable marker such as drug resistance.

The viral vector genome is typically constructed in a plasmid form that can be transfected into a packaging or producer cell line. Any of a variety of known methods can be used to produce retroviral particles whose genome contains an RNA copy of the viral vector genome. In some embodiments, at least two components are involved in making a virus-based gene delivery system: first, packaging plasmids, encompassing the structural proteins as well as the enzymes necessary to generate a viral vector particle, and second, the viral vector itself, i.e., the genetic material to be transferred. Biosafety safeguards can be introduced in the design of one or both of these components.

In some embodiments, the packaging plasmid can contain all retroviral, such as HIV-1, proteins other than envelope proteins (Naldini et al., 1998). In other embodiments, viral vectors can lack additional viral genes, such as those that are associated with virulence, e.g., vpr, vif, vpu and nef, and/or Tat, a primary transactivator of HIV. In some embodiments, lentiviral vectors, such as HIV-based lentiviral vectors, comprise only three genes of the parental virus: gag, pol and rev, which reduces or eliminates the possibility of reconstitution of a wild-type virus through recombination.

In some embodiments, the viral vector genome is introduced into a packaging cell line that contains all the components necessary to package viral genomic RNA, transcribed from the viral vector genome, into viral particles. Alternatively, the viral vector genome may comprise one or more genes encoding viral components in addition to the one or more sequences, e.g., recombinant nucleic acids, of interest. In some aspects, in order to prevent replication of the genome in the target cell, however, endogenous viral genes required for replication are removed and provided separately in the packaging cell line.

In some embodiments, a packaging cell line is transfected with one or more plasmid vectors containing the components necessary to generate the particles. In some embodiments, a packaging cell line is transfected with a plasmid containing the viral vector genome, including the LTRs, the cis-acting packaging sequence and the sequence of interest, i.e. a nucleic acid encoding an antigen receptor, such as a CAR; and one or more helper plasmids encoding the virus enzymatic and/or structural components, such as Gag, pol and/or rev. In some embodiments, multiple vectors are utilized to separate the various genetic components that generate the retroviral vector particles. In some such embodiments, providing separate vectors to the packaging cell reduces the chance of recombination events that might otherwise generate replication competent viruses. In some embodiments, a single plasmid vector having all of the retroviral components can be used.

In some embodiments, the retroviral vector particle, such as lentiviral vector particle, is pseudotyped to increase the transduction efficiency of host cells. For example, a retroviral vector particle, such as a lentiviral vector particle, in some embodiments is pseudotyped with a VSV-G glycoprotein, which provides a broad cell host range extending the cell types that can be transduced. In some embodiments, a packaging cell line is transfected with a plasmid or polynucleotide encoding a non-native envelope glycoprotein, such as to include xenotropic, polytropic or amphotropic envelopes, such as Sindbis virus envelope, GALV or VSV-G.

In some embodiments, the packaging cell line provides the components, including viral regulatory and structural proteins, that are required in trans for the packaging of the viral genomic RNA into lentiviral vector particles. In some embodiments, the packaging cell line may be any cell line that is capable of expressing lentiviral proteins and producing functional lentiviral vector particles. In some aspects, suitable packaging cell lines include 293 (ATCC CCL X), 293T, HeLA (ATCC CCL 2), D17 (ATCC CCL 183), MDCK (ATCC CCL 34), BHK (ATCC CCL-10) and Cf2Th (ATCC CRL 1430) cells.

In some embodiments, the packaging cell line stably expresses the viral protein(s). For example, in some aspects, a packaging cell line containing the gag, pol, rev and/or other structural genes but without the LTR and packaging components can be constructed. In some embodiments, a packaging cell line can be transiently transfected with nucleic acid molecules encoding one or more viral proteins along with the viral vector genome containing a nucleic acid molecule encoding a heterologous protein, and/or a nucleic acid encoding an envelope glycoprotein.

In some embodiments, the viral vectors and the packaging and/or helper plasmids are introduced via transfection or infection into the packaging cell line. The packaging cell line produces viral vector particles that contain the viral vector genome. Methods for transfection or infection are well known. Non-limiting examples include calcium phosphate, DEAE-dextran and lipofection methods, electroporation and microinjection.

When a recombinant plasmid and the retroviral LTR and packaging sequences are introduced into a special cell line (e.g., by calcium phosphate precipitation for example), the packaging sequences may permit the RNA transcript of the recombinant plasmid to be packaged into viral particles, which then may be secreted into the culture media. The media containing the recombinant retroviruses in some embodiments is then collected, optionally concentrated, and used for gene transfer. For example, in some aspects, after co-transfection of the packaging plasmids and the transfer vector to the packaging cell line, the viral vector particles are recovered from the culture media and titered by standard methods used by those of skill in the art.

In some embodiments, a retroviral vector, such as a lentiviral vector, can be produced in a packaging cell line, such as an exemplary HEK 293T cell line, by introduction of plasmids to allow generation of lentiviral particles. In some embodiments, a packaging cell is transfected and/or contains a polynucleotide encoding gag and pol, and a polynucleotide encoding a recombinant receptor, such as an antigen receptor, for example, a CAR. In some embodiments, the packaging cell line is optionally and/or additionally transfected with and/or contains a polynucleotide encoding a rev protein. In some embodiments, the packaging cell line is optionally and/or additionally transfected with and/or contains a polynucleotide encoding a non-native envelope glycoprotein, such as VSV-G. In some such embodiments, approximately two days after transfection of cells, e.g., HEK 293T cells, the cell supernatant contains recombinant lentiviral vectors, which can be recovered and titered.

Recovered and/or produced retroviral vector particles can be used to transduce target cells using the methods as described. Once in the target cells, the viral RNA is reverse-transcribed, imported into the nucleus and stably integrated into the host genome. One or two days after the integration of the viral RNA, the expression of the recombinant protein, e.g., antigen receptor, such as CAR, can be detected.

In some embodiments, the provided methods involve methods of transducing cells by contacting, e.g., incubating, a cell composition comprising a plurality of cells with a viral particle. In some embodiments, the cells to be transfected or transduced are or comprise primary cells obtained from a subject, such as cells enriched and/or selected from a subject.

In some embodiments, the concentration of cells to be transduced of the composition is from 1.0×10⁵ cells/mL to 1.0×10⁸ cells/mL or from about 1.0×10⁵ cells/mL to about 1.0×10⁸ cells/mL, such as at least or about at least or about 1.0×10⁵ cells/mL, 5×10⁵ cells/mL, 1×10⁶ cells/mL, 5×10⁶ cells/mL, 1×10⁷ cells/mL, 5×10⁷ cells/mL or 1×10⁸ cells/mL.

In some embodiments, the viral particles are provided at a certain ratio of copies of the viral vector particles or infectious units (IU) thereof, per total number of cells to be transduced (IU/cell). For example, in some embodiments, the viral particles are present during the contacting at or about or at least at or about 0.5, 1, 2, 3, 4, 5, 10, 15, 20, 30, 40, 50, or 60 IU of the viral vector particles per one of the cells.

In some embodiments, the titer of viral vector particles is between or between about 1×10⁶ IU/mL and 1×10⁸ IU/mL, such as between or between about 5×10⁶ IU/mL and 5×10⁷ IU/mL, such as at least 6×10⁶ IU/mL, 7×10⁶ IU/mL, 8×10⁶ IU/mL, 9×10⁶ IU/mL, 1×10⁷ IU/mL, 2×10⁷ IU/mL, 3×10⁷ IU/mL, 4×10⁷ IU/mL, or 5×10⁷ IU/mL.

In some embodiments, transduction can be achieved at a multiplicity of infection (MOI) of less than 100, such as generally less than 60, 50, 40, 30, 20, 10, 5 or less.

In some embodiments, the method involves contacting or incubating, the cells with the viral particles. In some embodiments, the contacting is for 30 minutes to 72 hours, such as 30 minute to 48 hours, 30 minutes to 24 hours or 1 hour to 24 hours, such as at least or about at least or about 30 minutes, 1 hour, 2 hours, 6 hours, 12 hours, 24 hours, 36 hours or more.

In some embodiments, contacting is performed in solution. In some embodiments, the cells and viral particles are contacted in a volume of from 0.5 mL to 500 mL or from about 0.5 mL to about 500 mL, such as from or from about 0.5 mL to 200 mL, 0.5 mL to 100 mL, 0.5 mL to 50 mL, 0.5 mL to 10 mL, 0.5 mL to 5 mL, 5 mL to 500 mL, 5 mL to 200 mL, 5 mL to 100 mL, 5 mL to 50 mL, 5 mL to 10 mL, 10 mL to 500 mL, 10 mL to 200 mL, 10 mL to 100 mL, 10 mL to 50 mL, 50 mL to 500 mL, 50 mL to 200 mL, 50 mL to 100 mL, 100 mL to 500 mL, 100 mL to 200 mL or 200 mL to 500 mL.

In certain embodiments, the input cells are treated, incubated, or contacted with particles that comprise binding molecules that bind to or recognize the recombinant receptor that is encoded by the viral DNA.

In some embodiments, the incubation of the cells with the viral vector particles results in or produces an output composition comprising cells transduced with the viral vector particles.

b. Non-Viral Vectors

In some embodiments, recombinant nucleic acids are transferred into T cells via electroporation (see, e.g., Chicaybam et al, (2013) PLoS ONE 8(3): e60298 and Van Tedeloo et al. (2000) Gene Therapy 7(16): 1431-1437). In some embodiments, recombinant nucleic acids are transferred into T cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506: 115-126). Other methods of introducing and expressing genetic material in immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)).

Other approaches and vectors for transfer of the nucleic acids encoding the recombinant products are those described, e.g., in international patent application, Publication No.: WO2014055668, and U.S. Pat. No. 7,446,190.

In some embodiments, recombinant nucleic acids are transferred into T cells via transposons. Transposons (transposable elements), are mobile segments of DNA that can move from one locus to another within genomes. These elements move via a conservative, “cut-and-paste” mechanism: the transposase catalyzes the excision of the transposon from its original location and promotes its reintegration elsewhere in the genome. Transposase-deficient elements can be mobilized if the transposase is provided by another transposase gene. Thus, transposons can be utilized to incorporate a foreign DNA into a host genome without the use of a viral transduction system. Examples of transposons suitable for use with mammalian cells, e.g., human primary leukocytes, include but are not limited to Sleeping Beauty and PiggyBacs.

Transposon-based transfection is a two-component system consisting of a transposase and a transposon. In some embodiments, the system comprises a transposon is engineered to comprise a foreign DNA (also referred herein as cargo DNA), e.g., a gene encoding a recombinant receptor, that is flanked by inverted repeat/direct repeat (IR/DR) sequences that are recognized by an accompanying transposase. In some embodiments, a non-viral plasmid encodes a transposase under the control of a promoter. Transfection of the plasmid into a host cell results in a transitory expression of the transposase, thus for an initial period following transfection, the transposase is expressed at sufficiently levels to integrate the transposon into the genomic DNA. In some embodiments, the transposase itself is not integrated into the genomic DNA, and therefor expression of the transposase decreases over time. In some embodiments, the transposase expression is expressed by the host cell at levels sufficient to integrate a corresponding transposon for less than about 4 hours, less than about 8 hours, less than about 12 hours, less than about 24 hours, less than about 2 days, less than about 3 days, less than about 4 days, less than about 5 days, less than about 6 days, less than about 7 days, less than about 2 weeks, less than about 3 weeks, less than about 4 weeks, less than about weeks, or less than about 8 weeks. In some embodiments, the cargo DNA that is introduced into the host's genome is not subsequently removed from the host's genome, at least because the host dose not express an endogenous transposase capable of excising the cargo DNA.

Sleeping Beauty (SB) is a synthetic member of the Tc/1-mariner superfamily of transposons, reconstructed from dormant elements harbored in the salmonid fish genome. SB transposon-based transfection is a two-component system consisting of a transposase and a transposon containing inverted repeat/direct repeat (IR/DR) sequences that result in precise integration into a TA dinucleotide. The transposon is designed with an expression cassette of interest flanked by IR/DRs. The SB transposase binds specific binding sites that are located on the IR of the Sleeping beauty transposon. The SB transposase mediates integration of the transposon, a mobile element encoding a cargo sequence flanked on both sides by inverted terminal repeats that harbor binding sites for the catalytic enzyme (SB). Stable expression results when SB inserts gene sequences into vertebrate chromosomes at a TA target dinucleotide through a cut-and-paste mechanism. This system has been used to engineer a variety of vertebrate cell types, including primary human peripheral blood leukocytes. In some embodiments, the cells are contacted, incubated, and/or treated with an SB transposon comprising a cargo gene, e.g., a gene encoding a recombinant receptor or a CAR, flanked by SB IR sequences. In particular embodiments, the cells to be transfected are contacted, incubated, and/or treated with a plasmid comprising an SB transposon comprising a cargo gene, e.g., a gene encoding a CAR, flanked by SB IR sequences. In certain embodiments, the plasmid further comprises a gene encoding an SB transposase that is not flanked by SB IR sequences.

PiggyBac (PB) is another transposon system that can be used to integrate cargo DNA into a host's, e.g., a human's, genomic DNA. The PB transposase recognizes PB transposon-specific inverted terminal repeat sequences (ITRs) located on both ends of the transposon and efficiently moves the contents from the original sites and efficiently integrates them into TTAA chromosomal sites. The PB transposon system enables genes of interest between the two ITRs in the PB vector to be mobilized into target genomes. The PB system has been used to engineer a variety of vertebrate cell types, including primary human cells. In some embodiments, the cells to be transfected are contacted, incubated, and/or treated with an PB transposon comprising a cargo gene, e.g., a gene encoding a CAR, flanked by PB IR sequences. In particular embodiments, the cells to be transfected are contacted, incubated, and/or treated with a plasmid comprising a PB transposon comprising a cargo gene, e.g., a gene encoding a CAR, flanked by PB IR sequences. In certain embodiments, the plasmid further comprises a gene encoding an SB transposase that is not flanked by PB IR sequences.

In some embodiments, the various elements of the transposon/transposase the employed in the subject methods, e.g., SB or PB vector(s), may be produced by standard methods of restriction enzyme cleavage, ligation and molecular cloning. One protocol for constructing the subject vectors includes the following steps. First, purified nucleic acid fragments containing desired component nucleotide sequences as well as extraneous sequences are cleaved with restriction endonucleases from initial sources, e.g., a vector comprising the transposase gene. Fragments containing the desired nucleotide sequences are then separated from unwanted fragments of different size using conventional separation methods, e.g., by agarose gel electrophoresis. The desired fragments are excised from the gel and ligated together in the appropriate configuration so that a circular nucleic acid or plasmid containing the desired sequences, e.g., sequences corresponding to the various elements of the subject vectors, as described above is produced. Where desired, the circular molecules so constructed are then amplified in a prokaryotic host, e.g., E. coli. The procedures of cleavage, plasmid construction, cell transformation and plasmid production involved in these steps are well known to one skilled in the art and the enzymes required for restriction and ligation are available commercially. (See, for example, R. Wu, Ed., Methods in Enzymology, Vol. 68, Academic Press, N.Y. (1979); T. Maniatis, E. F. Fritsch and J. Sambrook, Molecular Cloning: A Laboratory Manual, Cold Spring Harbor Laboratory Press, Cold Spring Harbor, N.Y. (1982); Catalog 1982-83, New England Biolabs, Inc.; Catalog 1982-83, Bethesda Research Laboratories, Inc. An example of how to construct the vectors employed in the subject methods is provided in the Experimental section, infra. The preparation of a representative Sleeping Beauty transposon system is also disclosed in WO 98/40510 and WO 99/25817).

In some embodiments, transduction with transposons is performed with a plasmid that comprises a transposase gene and a plasmid that comprises a transposon that contains a cargo DNA sequence that is flanked by inverted repeat/direct repeat (IR/DR) sequences that are recognized by the transposase. In certain embodiments, the cargo DNA sequence encodes a heterologous protein, e.g., a recombinant T cell receptor or a CAR. In some embodiments, the plasmid comprises transposase and the transposon. In some embodiments, the transposase is under control of a ubiquitous promoter, or any promoter suitable to drive expression of the transposase in the target cell. Ubiquitous promoters include, but are not limited to, EF1a, CMB, SV40, PGK1, Ube, human P-actin, CAG, TRE, UAS, Ac5, CaMKIIa, and U6. In some embodiments, the cargo DNA comprises a selection cassette allowing for the selection of cells with stable integration of the cargo DNA into the genomic DNA. Suitable selection cassettes include, but are not limited to, selection cassettes encoding a kanamycin resistance gene, spectinomycin resistance gene, streptomycin resistance gene, ampicillin resistance gene, carbenicillin resistance gene, hygromycin resistance gene, bleomycin resistance gene, erythromycin resistance gene, and polymyxin B resistance gene.

In some embodiments, the components for transduction with a transposon, e.g., plasmids comprising an SB transposase and SB transposon, are introduced into the target cell. Any convenient protocol may be employed, where the protocol may provide for in vitro or in vivo introduction of the system components into the target cell, depending on the location of the target cell. For example, where the target cell is an isolated cell, the system may be introduced directly into the cell under cell culture conditions permissive of viability of the target cell, e.g., by using standard transformation techniques. Such techniques include, but are not necessarily limited to: viral infection, transformation, conjugation, protoplast fusion, electroporation, particle gun technology, calcium phosphate precipitation, direct microinjection, viral vector delivery, and the like. The choice of method is generally dependent on the type of cell being transformed and the circumstances under which the transformation is taking place (i.e. in vitro, ex vivo, or in vivo). A general discussion of these methods can be found in Ausubel, et al, Short Protocols in Molecular Biology, 3rd ed., Wiley & Sons, 1995.

In some embodiments, the SB transposon and the SB transposase source are introduced into a target cell of a multicellular organism, e.g., a mammal or a human, under conditions sufficient for excision of the inverted repeat flanked nucleic acid from the vector carrying the transposon and subsequent integration of the excised nucleic acid into the genome of the target cell. Some embodiments further comprise a step of ensuring that the requisite transposase activity is present in the target cell along with the introduced transposon. Depending on the structure of the transposon vector itself, i.e. whether or not the vector includes a region encoding a product having transposase activity, the method may further include introducing a second vector into the target cell which encodes the requisite transposase activity.

In some embodiments, the amount of vector nucleic acid comprising the transposon and the amount of vector nucleic acid encoding the transposase that is introduced into the cell is sufficient to provide for the desired excision and insertion of the transposon nucleic acid into the target cell genome. As such, the amount of vector nucleic acid introduced should provide for a sufficient amount of transposase activity and a sufficient copy number of the nucleic acid that is desired to be inserted into the target cell. The amount of vector nucleic acid that is introduced into the target cell varies depending on the efficiency of the particular introduction protocol that is employed, e.g., the particular ex vivo administration protocol that is employed.

Once the vector DNA has entered the target cell in combination with the requisite transposase, the nucleic acid region of the vector that is flanked by inverted repeats, i.e. the vector nucleic acid positioned between the Sleeping Beauty transposase recognized inverted repeats, is excised from the vector via the provided transposase and inserted into the genome of the targeted cell. As such, introduction of the vector DNA into the target cell is followed by subsequent transposase mediated excision and insertion of the exogenous nucleic acid carried by the vector into the genome of the targeted cell. In particular embodiments, the vector is integrated into the genomes of at least 1%, at least 2%, at least 3%, at least 4%, at least 5%, at least 6% at least 7% at least 8%, at least 9%, at least 10%, at least 15%, or at least 20% of the cells that are transfected with the SB transposon and/or SB transposase. In some embodiments, integration of the nucleic acid into the target cell genome is stable, i.e., the vector nucleic acid remains present in the target cell genome for more than a transient period of time and is passed on a part of the chromosomal genetic material to the progeny of the target cell.

In certain embodiments, the transposons are used to integrate nucleic acids, i.e. polynucleotides, of various sizes into the target cell genome. In some embodiments, the size of DNA that is inserted into a target cell genome using the subject methods ranges from about 0.1 kb to 200 kb, from about 0.5 kb to 100 kb, from about 1.0 kb to about 8.0 kb, from about 1.0 to about 200 kb, from about 1.0 to about 10 kb, from about 10 kb to about 50 kb, from about 50 kb to about 100 kb, or from about 100 kb to about 200 kb. In some embodiments, the size of DNA that is inserted into a target cell genome using the subject methods ranges from about from about 1.0 kb to about 8.0 kb. In some embodiments, the size of DNA that is inserted into a target cell genome using the subject methods ranges from about 1.0 to about 200 kb. In particular embodiments, the size of DNA that is inserted into a target cell genome using the subject methods ranges from about 1.0 kb to about 8.0 kb.

D. Cultivation and/or Expansion of Cells

In some embodiments, the provided methods include one or more steps for cultivating cells, e.g., cultivating cells under conditions that promote proliferation and/or expansion. In some embodiments, cells are cultivated under conditions that promote proliferation and/or expansion subsequent to a step of genetically engineering, e.g., introducing a recombinant polypeptide to the cells by transduction or transfection. In particular embodiments, the cells are cultivated after the cells have been incubated under stimulating conditions and transduced or transfected with a recombinant polynucleotide, e.g., a polynucleotide encoding a recombinant receptor. In some embodiments, the cultivation produces one or more cultivated compositions of enriched T cells.

In certain embodiments, one or more compositions of enriched T cells, including stimulated and transduced T cells, such as separate compositions of such CD4+ and CD8+ T cells, are cultivated, e.g., under conditions that promote proliferation and/or expansion, prior to formulating the cells. In some aspects, the methods of cultivation, such as for promoting proliferation and/or expansion include methods provided herein. In particular embodiments, one or more compositions of enriched T cells are cultivated after the one or more compositions have been engineered, e.g., transduced or transfected. In particular embodiments, the one or more compositions are engineered compositions. In particular embodiments, the one or more engineered compositions have been previously cryofrozen and stored, and are thawed prior to cultivating.

In certain embodiments, the one or more compositions of engineered T cells are or include two separate compositions of enriched T cells. In particular embodiments, two separate compositions of enriched T cells, e.g., two separate compositions of enriched T cells selected, isolated, and/or enriched from the same biological sample, that are introduced with a recombinant receptor (e.g., CAR), are separately cultivated under conditions that promote proliferation and/or expansion of the cells. In some embodiments, the conditions are stimulating conditions. In certain embodiments, the two separate compositions include a composition of enriched CD4+ T cells, such as engineered CD4+ T cells that were introduced with the nucleic acid encoding the recombinant receptor and/or that express the recombinant receptor. In particular embodiments, the two separate compositions include a composition of enriched CD8+ T cells, such as engineered CD8+ T cells that were introduced with the nucleic acid encoding the recombinant receptor and/or that express the recombinant receptor. In some embodiments, two separate compositions of enriched CD4+ T cells and enriched CD8+ T cells, such as engineered CD4+ T cells and engineered CD8+ T cells, are separately cultivated, e.g., under conditions that promote proliferation and/or expansion. In some embodiments, a single composition of enriched T cells is cultivated. In certain embodiments, the single composition is a composition of enriched CD4+ T cells. In some embodiments, the single composition is a composition of enriched CD4+ and CD8+ T cells that have been combined from separate compositions prior to the cultivation.

In some embodiments, the composition of enriched CD4+ T cells, such as engineered CD4+ T cells, that is cultivated, e.g., under conditions that promote proliferation and/or expansion, includes at least 60%, at least 65%, at least 70%, at least 75%, at least 80%, at least 85%, at least 90%, at least 95%, at least 98%, at least 99%, at least 99.5%, at least 99.9%, or at or at about 100% CD4+ T cells. In some embodiments, the composition includes at least 30%, at least 40%, at least 50%, at least 60%, at least 70%, at least 80%, at least 90%, at least 95%, at least 98%, at least 99%, at least 99.5%, at least 99.9%, or at or at about 100% CD4+ T cells that express the recombinant receptor and/or have been transduced or transfected with the recombinant polynucleotide encoding the recombinant receptor. In certain embodiments, the composition of enriched CD4+ T cells that is cultivated includes less than 40%, less than 35%, less than 30%, less than 25%, less than 20%, less than 15%, less than 10%, less than 5%, less than 1%, less than 0.1%, or less than 0.01% CD8+ T cells, and/or contains no CD8+ T cells, and/or is free or substantially free of CD8+ T cells.

In some embodiments, the composition of enriched CD8+ T cells, such as engineered CD8+ T cells, that is cultivated, e.g., under conditions that promote proliferation and/or expansion, includes at least 60%, at least 65%, at least 70%, at least 75%, at least 80%, at least 85%, at least 90%, at least 95%, at least 98%, at least 99%, at least 99.5%, at least 99.9%, or at or at about 100% CD8+ T cells. In particular embodiments, the composition includes at least 30%, at least 40%, at least 50%, at least 60%, at least 70%, at least 80%, at least 90%, at least 95%, at least 98%, at least 99%, at least 99.5%, at least 99.9%, or at or at about 100% CD8+ T cells that express the recombinant receptor and/or have been transduced or transfected with the recombinant polynucleotide encoding the recombinant receptor. In certain embodiments, the composition of enriched CD8+ T cells that is incubated under stimulating conditions includes less than 40%, less than 35%, less than 30%, less than 25%, less than 20%, less than 15%, less than 10%, less than 5%, less than 1%, less than 0.1%, or less than 0.01% CD4+ T cells, and/or contains no CD4+ T cells, and/or is free or substantially free of CD4+ T cells.

In some embodiments, separate compositions of enriched CD4+ and CD8+ T cells, such as separate compositions of engineered CD4+ and engineered CD8+ T cells, are combined into a single composition and are cultivated, e.g., under conditions that promote proliferation and/or expansion. In certain embodiments, separate cultivated compositions of enriched CD4+ and enriched CD8+ T cells are combined into a single composition after the cultivation has been performed and/or completed. In particular embodiments, separate compositions of enriched CD4+ and CD8+ T cells, such as separate compositions of engineered CD4+ and engineered CD8+ T cells, are separately cultivated, e.g., under conditions that promote proliferation and/or expansion.

In some embodiments, the cells, e.g., the engineered cells are cultivated in a volume of media that is, is about, or is at least 100 mL, 200 mL, 300 mL, 400 mL, 500 mL, 600 mL, 700 mL, 800 mL, 900 mL, 1,000 mL, 1,200 mL, 1,400 mL, 1,600 mL, 1,800 mL, 2,000 mL, 2,200 mL, or 2,400 mL. In some embodiments, the cells are cultivated at an initial volume that is later adjusted to a different volume. In particular embodiments, the volume is later adjusted during the cultivation. In particular embodiments, the volume is increased from the initial volume during the cultivation. In certain embodiments, the volume is increased when the cells achieve a density during the cultivation. In certain embodiment, the initial volume is or is about 500 mL.

In particular embodiments, the volume is increased from the initial volume when the cells achieve a density or concentration during the cultivation. In particular embodiments, the volume is increased when the cells achieve a density and/or concentration of, of about, or of at least 0.1×10⁶ cells/ml, 0.2×10⁶ cells/ml, 0.4×10⁶ cells/ml, 0.6×10⁶ cells/ml, 0.8×10⁶ cells/ml, 1×10⁶ cells/ml, 1.2×10⁶ cells/ml, 1.4×10⁶ cells/ml, 1.6×10⁶ cells/ml, 1.8×10⁶ cells/ml, 2.0×10⁶ cells/ml, 2.5×10⁶ cells/ml, 3.0×10⁶ cells/ml, 3.5×10⁶ cells/ml, 4.0×10⁶ cells/ml, 4.5×10⁶ cells/ml, 5.0×10⁶ cells/ml, 6×10⁶ cells/ml, 8×10⁶ cells/ml, or 10×10⁶ cells/ml. In some embodiments, the volume is increased from the initial volume when the cells achieve a density and/or concentration of, of at least, or of about 0.6×10⁶ cells/ml. In some embodiments, the density and/or concentration is of viable cells in the culture. In particular embodiments, the volume is increased when the cells achieve a density and/or concentration of, of about, or of at least 0.1×10⁶ viable cells/ml, 0.2×10⁶ viable cells/ml, 0.4×10⁶ viable cells/ml, 0.6×10⁶ viable cells/ml, 0.8×10⁶ viable cells/ml, 1×10⁶ viable cells/ml, 1.2×10⁶ viable cells/ml, 1.4×10⁶ viable cells/ml, 1.6×10⁶ viable cells/ml, 1.8×10⁶ viable cells/ml, 2.0×10⁶ viable cells/ml, 2.5×10⁶ viable cells/ml, 3.0×10⁶ viable cells/ml, 3.5×10⁶ viable cells/ml, 4.0×10⁶ viable cells/ml, 4.5×10⁶ viable cells/ml, 5.0×10⁶ viable cells/ml, 6×10⁶ viable cells/ml, 8×10⁶ viable cells/ml, or 10×10⁶ viable cells/ml. In some embodiments, the volume is increased from the initial volume when the viable cells achieve a density and/or concentration of, of at least, or of about 0.6×10⁶ viable cells/ml. In some embodiments, density and/or concentration of the cells or viable cells can be determined or monitored during the cultivation, such as by using methods as described, including optical methods, including digital holography microscopy (DHM) or differential digital holography microscopy (DDHM).

In some embodiments, the cells achieve a density and/or concentration, and the volume is increased by, by about, or by at least 100 mL, 200 mL, 300 mL, 400 mL, 500 mL, 600 mL, 700 mL, 800 mL, 900 mL, 1,000 mL, 1,200 mL, 1,400 mL, 1,600 mL, 1,800 mL, 2,000 mL, 2,200 mL or 2,400 mL. In some embodiments, the volume is increased by 500 mL. In particular embodiments, the volume is increased to a volume of, of about, or of at least 500 mL, 600 mL, 700 mL, 800 mL, 900 mL, 1,000 mL, 1,200 mL, 1,400 mL, 1,600 mL, 1,800 mL, 2,000 mL, 2,200 mL or 2,400 mL. In certain embodiments, the volume is increased to a volume of 1,000 mL. In certain embodiments, the volume is increase at a rate of, of at least, or of about 5 mL, 10 mL, 20 mL, 25 mL, 30 mL, 40 mL, 50 mL, 60 mL, 70 mL, 75 mL, 80 mL, 90 mL, or 100 mL, every 1, 2, 3, 4, 5, 6, 7, 8, 9, or 10 minutes. In certain embodiments, the rate is or is about 50 mL every 8 minutes.

In some embodiments, a composition of enriched T cells, such as engineered T cells, is cultivated under conditions that promote proliferation and/or expansion. In some embodiments, such conditions may be designed to induce proliferation, expansion, activation, and/or survival of cells in the population. In particular embodiments, the stimulating conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to promote growth, division, and/or expansion of the cells.

In some embodiments, the cultivation is performed under conditions that generally include a temperature suitable for the growth of primary immune cells, such as human T lymphocytes, for example, at least about 25 degrees Celsius, generally at least about 30 degrees, and generally at or about 37 degrees Celsius. In some embodiments, the composition of enriched T cells is incubated at a temperature of 25 to 38° C., such as 30 to 37° C., for example at or about 37° C.±2° C. In some embodiments, the incubation is carried out for a time period until the culture, e.g., cultivation or expansion, results in a desired or threshold density, concentration, number or dose of cells. In some embodiments, the incubation is carried out for a time period until the culture, e.g., cultivation or expansion, results in a desired or threshold density, concentration, number or dose of viable cells. In some embodiments, the incubation is greater than or greater than about or is for about or 24 hours, 48 hours, 72 hours, 96 hours, 5 days, 6 days, 7 days, 8 days, 9 days or more. In some embodiments, density, concentration and/or number or dose of the cells can be determined or monitored during the cultivation, such as by using methods as described, including optical methods, including digital holography microscopy (DHM) or differential digital holography microscopy (DDHM).

In some embodiments, the stimulatory reagent is removed and/or separated from the cells prior to the cultivation. In certain embodiments, the stimulatory agent is removed and/or separated from the cells subsequent to the engineering and prior to cultivating the engineered cells, e.g., under conditions that promote proliferation and/or expansion. In some embodiments, the stimulatory reagent is a stimulatory reagent that is described herein, e.g., in Section II-B-1. In particular embodiments, the stimulatory reagent is removed and/or separated from the cells as described herein, e.g., in Section II-B-2.

In particular embodiments, a composition of enriched T cells, such as engineered T cells, for example separate compositions of engineered CD4+ T cells and engineered CD8+ T cells, is cultivated in the presence of one or more cytokines. In certain embodiments, the one or more cytokines are recombinant cytokines. In particular embodiments, the one or more cytokines are human recombinant cytokines. In certain embodiments, the one or more cytokines bind to and/or are capable of binding to receptors that are expressed by and/or are endogenous to T cells. In particular embodiments, the one or more cytokines is or includes a member of the 4-alpha-helix bundle family of cytokines. In some embodiments, members of the 4-alpha-helix bundle family of cytokines include, but are not limited to, interleukin-2 (IL-2), interleukin-4 (IL-4), interleukin-7 (IL-7), interleukin-9 (IL-9), interleukin 12 (IL-12), interleukin 15 (IL-15), granulocyte colony-stimulating factor (G-CSF), and granulocyte-macrophage colony-stimulating factor (GM-CSF). In some embodiments, the one or more cytokines is or includes IL-15. In particular embodiments, the one or more cytokines is or includes IL-7. In particular embodiments, the one or more cytokines is or includes recombinant IL-2.

In particular embodiments, the composition of enriched CD4+ T cells, such as engineered CD4+ T cells, is cultivated with recombinant IL-2. In some embodiments, cultivating a composition of enriched CD4+ T cells, such as engineered CD4+ T cells, in the presence of recombinant IL-2 increases the probability or likelihood that the CD4+ T cells of the composition will continue to survive, grow, expand, and/or activate during the cultivation step and throughout the process. In some embodiments, cultivating the composition of enriched CD4+ T cells, such as engineered CD4+ T cells, in the presence of recombinant IL-2 increases the probability and/or likelihood that an output composition of enriched CD4+ T cells, e.g., engineered CD4+ T cells suitable for cell therapy, will be produced from the composition of enriched CD4+ T cells by at least 0.5%, at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 30%, at least 40%, at least 50%, at least 60%, at least 70%, at least 80%, at least 90%, at least 95%, at least 100%, or at least 200% CD4+ as compared to an alternative and/or exemplary method that does not cultivate the composition of enriched CD4+ T cells in the presence of recombinant IL-2.

In some embodiments, the cells, such as separate compositions of engineered CD4+ T cells and engineered CD8+ T cells, are cultivated with a cytokine, e.g., a recombinant human cytokine, at a concentration of between 1 IU/ml and 2,000 IU/ml, between 10 IU/ml and 100 IU/ml, between 50 IU/ml and 500 IU/ml, between 100 IU/ml and 200 IU/ml, between 500 IU/ml and 1400 IU/ml, between 250 IU/ml and 500 IU/ml, or between 500 IU/ml and 2,500 IU/ml.

In some embodiments, a composition of enriched of T cells, such as separate compositions of engineered CD4+ T cells and CD8+ T cells, is cultivated with recombinant IL-2, e.g., human recombinant IL-2, at a concentration between 2 IU/ml and 500 IU/ml, between 10 IU/ml and 250 IU/ml, between 100 IU/ml and 500 IU/ml, or between 100 IU/ml and 400 IU/ml. In particular embodiments, the composition of enriched T cells is cultivated with IL-2 at a concentration at or at about 50 IU/ml, 75 IU/ml, 100 IU/ml, 125 IU/ml, 150 IU/ml, 175 IU/ml, 200 IU/ml, 225 IU/ml, 250 IU/ml, 300 IU/ml, or 400 IU/ml. In some embodiments, the composition of enriched T cells is cultivated with recombinant IL-2 at a concentration of 200 IU/ml. In some embodiments, the composition of enriched T cells is a composition of enriched CD4+ T cells, such as a composition of engineered CD4+ T cells. In particular embodiments, the composition of enriched T cells is a composition of enriched CD8+ T cells, such as a composition of engineered CD8+ T cells.

In some embodiments, a composition of enriched T cells, such as separate compositions of engineered CD4+ T cells and CD8+ T cells, is cultivated with IL-7, e.g., human recombinant IL-7, at a concentration between 10 IU/ml and 5,000 IU/ml, between 500 IU/ml and 2,000 IU/ml, between 600 IU/ml and 1,500 IU/ml, between 500 IU/ml and 2,500 IU/ml, between 750 IU/ml and 1,500 IU/ml, or between 1,000 IU/ml and 2,000 IU/ml. In particular embodiments, the composition of enriched T cells is cultivated with IL-7 at a concentration at or at about 100 IU/ml, 200 IU/ml, 300 IU/ml, 400 IU/ml, 500 IU/ml, 600 IU/ml, 700 IU/ml, 800 IU/ml, 900 IU/ml, 1,000 IU/ml, 1,200 IU/ml, 1,400 IU/ml, or 1,600 IU/ml. In some embodiments, the cells are cultivated in the presence of recombinant IL-7 at a concertation of or of about 1,200 IU/ml. In some embodiments, the composition of enriched T cells is a composition of enriched CD4+ T cells, such as engineered CD4+ T cells.

In some embodiments, a composition of enriched T cells, such as separate compositions of engineered CD4+ T cells and CD8+ T cells, is cultivated with IL-15, e.g., human recombinant IL-15, at a concentration between 0.1 IU/ml and 200 IU/ml, between 1 IU/ml and 50 IU/ml, between 5 IU/ml and 25 IU/ml, between 25 IU/ml and 50 IU/ml, between 5 IU/ml and 15 IU/ml, or between 10 IU/ml and 00 IU/ml. In particular embodiments, the composition of enriched T cells is cultivated with IL-15 at a concentration at or at about 1 IU/ml, 2 IU/ml, 3 IU/ml, 4 IU/ml, 5 IU/ml, 6 IU/ml, 7 IU/ml, 8 IU/ml, 9 IU/ml, 10 IU/ml, 11 IU/ml, 12 IU/ml, 13 IU/ml, 14 IU/ml, 15 IU/ml, 20 IU/ml, 25 IU/ml, 30 IU/ml, 40 IU/ml, 50 IU/ml, 100 IU/ml, or 200 IU/ml. In particular embodiments, a composition of enriched T cells is cultivated with recombinant IL-15 at a concentration of 20 IU/ml. In some embodiments, the composition of enriched T cells is a composition of enriched CD4+ T cells, such as engineered CD4+ T cells. In particular embodiments, the composition of enriched T cells is a composition of enriched CD8+ T cells, such as engineered CD8+ T cells.

In particular embodiments, a composition of enriched CD8+ T cells, such as engineered CD8+ T cells, is cultivated in the presence of IL-2 and/or IL-15, such as in amounts as described. In certain embodiments, a composition of enriched CD4+ T cells, such as engineered CD4+ T cells, is cultivated in the presence of IL-2, IL-7, and/or IL-15, such as in amounts as described. In some embodiments, the IL-2, IL-7, and/or IL-15 are recombinant. In certain embodiments, the IL-2, IL-7, and/or IL-15 are human. In particular embodiments, the one or more cytokines are or include human recombinant IL-2, IL-7, and/or IL-15.

In particular embodiments, the cultivation is performed in a closed system. In certain embodiments, the cultivation is performed in a closed system under sterile conditions. In particular embodiments, the cultivation is performed in the same closed system as one or more steps of the provided systems. In some embodiments the composition of enriched T cells is removed from a closed system and placed in and/or connected to a bioreactor for the cultivation. Examples of suitable bioreactors for the cultivation include, but are not limited to, GE Xuri W25, GE Xuri W5, Sartorius BioSTAT RM 20|50, Finesse SmartRocker Bioreactor Systems, and Pall XRS Bioreactor Systems. In some embodiments, the bioreactor is used to perfuse and/or mix the cells during at least a portion of the cultivation step.

In some embodiments, cells cultivated while enclosed, connected, and/or under control of a bioreactor undergo expansion during the cultivation more rapidly than cells that are cultivated without a bioreactor, e.g., cells that are cultivated under static conditions such as without mixing, rocking, motion, and/or perfusion. In some embodiments, cells cultivated while enclosed, connected, and/or under control of a bioreactor reach or achieve a threshold expansion, cell count, and/or density within 14 days, 10 days, 9 days, 8 days, 7 days, 6 days, 5 days, 4 days, 3 days, 2 days, 60 hours, 48 hours, 36 hours, 24 hours, or 12 hours. In some embodiments, cells cultivated while enclosed, connected, and/or under control of a bioreactor reach or achieve a threshold expansion, cell count, and/or density at least 50%, at least 60%, at least 70%, at least 80%, at least 90%, at least 95%, at least 100%, at least 150%, at least 1-fold, at least 2-fold, at least 3-fold, at least 4-fold, at least 5-fold than cells cultivated in an exemplary and/or alternative process where cells are not cultivated while enclosed, connected, and/or under control of a bioreactor.

In some embodiments, the mixing is or includes rocking and/or motioning. In some cases, the bioreactor can be subject to motioning or rocking, which, in some aspects, can increase oxygen transfer. Motioning the bioreactor may include, but is not limited to rotating along a horizontal axis, rotating along a vertical axis, a rocking motion along a tilted or inclined horizontal axis of the bioreactor or any combination thereof. In some embodiments, at least a portion of the incubation is carried out with rocking. The rocking speed and rocking angle may be adjusted to achieve a desired agitation. In some embodiments the rock angle is 20°, 19°, 18°, 170, 16°, 15°, 14°, 13°, 12°, 11°, 10°, 9°, 8°, 7°, 6°, 5°, 4°, 3°, 2° or 1. In certain embodiments, the rock angle is between 6-16°. In other embodiments, the rock angle is between 7-16°. In other embodiments, the rock angle is between 8-12°. In some embodiments, the rock rate is 1, 2, 3, 4, 5, 6, 7, 8, 9, 10, 11, 1 12, 13, 14 15, 16, 17, 18, 19, 20, 21, 22, 23, 24, 25, 26, 27, 28, 29, 30, 31, 32, 33, 34, 35, 36, 37, 38, 39, 40 rpm. In some embodiments, the rock rate is between 4 and 12 rpm, such as between 4 and 6 rpm, inclusive.

In some embodiments, the bioreactor maintains the temperature at or near 37° C. and CO₂ levels at or near 5% with a steady air flow at, at about, or at least 0.01 L/min, 0.05 L/min, 0.1 L/min, 0.2 L/min, 0.3 L/min, 0.4 L/min, 0.5 L/min, 1.0 L/min, 1.5 L/min, or 2.0 L/min or greater than 2.0 L/min. In certain embodiments, at least a portion of the cultivation is performed with perfusion, such as with a rate of 290 ml/day, 580 ml/day, and/or 1160 ml/day, e.g., depending on the timing in relation to the start of the cultivation and/or density of the cultivated cells. In some embodiments, at least a portion of the cell culture expansion is performed with a rocking motion, such as at an angle of between 5° and 10°, such as 6°, at a constant rocking speed, such as a speed of between 5 and 15 RPM, such as 6 RPM or 10 RPM.

In some embodiments, the at least a portion of the cultivation step is performed under constant perfusion, e.g., a perfusion at a slow steady rate. In some embodiments, the perfusion is or include an outflow of liquid e.g., used media, and an inflow of fresh media. In certain embodiments, the perfusion replaces used media with fresh media. In some embodiments, at least a portion of the cultivation is performed under perfusion at a steady rate of or of about or of at least 100 ml/day, 200 ml/day, 250 ml/day, 275 ml/day, 290 ml/day, 300 ml/day, 350 ml/day, 400 ml/day, 450 ml/day, 500 ml/day, 550 ml/day, 575 ml/day, 580 ml/day, 600 ml/day, 650 ml/day, 700 ml/day, 750 ml/day, 800 ml/day, 850 ml/day, 900 ml/day, 950 ml/day, 1000 ml/day, 1100 ml/day, 1160 ml/day, 1200 ml/day, 1400 ml/day, 1600 ml/day, 1800 ml/day, 2000 ml/day, 2200 ml/day, or 2400 ml/day.

In particular embodiments, cultivation is started under conditions with no perfusion, and perfusion started after a set and/or predetermined amount of time, such as or as about or at least 12 hours, 24 hours, 36 hours, 48 hours, 60 hours, 72 hours, or more than 72 hours after the start or initiation of the cultivation. In particular embodiments, perfusion is started when the density or concentration of the cells reaches a set or predetermined density or concentration. In some embodiments, the perfusion is started when the cultivated cells reach a density or concentration of, of about, or at least 0.1×10⁶ cells/ml, 0.2×10⁶ cells/ml, 0.4×10⁶ cells/ml, 0.6×10⁶ cells/ml, 0.8×10⁶ cells/ml, 1×10⁶ cells/ml, 1.2×10⁶ cells/ml, 1.4×10⁶ cells/ml, 1.6×10⁶ cells/ml, 1.8×10⁶ cells/ml, 2.0×10⁶ cells/ml, 2.5×10⁶ cells/ml, 3.0×10⁶ cells/ml, 3.5×10⁶ cells/ml, 4.0×10⁶ cells/ml, 4.5×10⁶ cells/ml, 5.0×10⁶ cells/ml, 6×10⁶ cells/ml, 8×10⁶ cells/ml, or 10×10⁶ cells/ml. In particular embodiments, perfusion is started when the density or concentration of viable cells reaches a set or predetermined density or concentration. In some embodiments, the perfusion is started when the cultivated viable cells reach a density or concentration of, of about, or at least 0.1×10⁶ viable cells/ml, 0.2×10⁶ viable cells/ml, 0.4×10⁶ viable cells/ml, 0.6×10⁶ viable cells/ml, 0.8×10⁶ viable cells/ml, 1×10⁶ viable cells/ml, 1.2×10⁶ viable cells/ml, 1.4×10⁶ viable cells/ml, 1.6×10⁶ viable cells/ml, 1.8×10⁶ viable cells/ml, 2.0×10⁶ viable cells/ml, 2.5×10⁶ viable cells/ml, 3.0×10⁶ viable cells/ml, 3.5×10⁶ viable cells/ml, 4.0×10⁶ viable cells/ml, 4.5×10⁶ viable cells/ml, 5.0×10⁶ viable cells/ml, 6×10⁶ viable cells/ml, 8×10⁶ viable cells/ml, or 10×10⁶ viable cells/ml.

In particular embodiments, the perfusion is performed at different speeds during the cultivation. For example, in some embodiments, the rate of the perfusion depends on the density and/or concentration of the cultivated cells. In certain embodiments, the rate of perfusion is increased when the cells reach a set or predetermined density or concentration. The perfusion rate may change, e.g., change from one steady perfusion rate to an increased steady perfusion rate, once, twice, three times, four times, five times, more than five times, more than ten times, more than 15 times, more than 20 times, more than 25 times, more than 50 times, or more than 100 times during the cultivation. In some embodiments, the steady perfusion rate increases when the cells reach a set or predetermined cell density or concentration of, of about, or at least 0.6×10⁶ cells/ml, 0.8×10⁶ cells/ml, 1×10⁶ cells/ml, 1.2×10⁶ cells/ml, 1.4×10⁶ cells/ml, 1.6×10⁶ cells/ml, 1.8×10⁶ cells/ml, 2.0×10⁶ cells/ml, 2.5×10⁶ cells/ml, 3.0×10⁶ cells/ml, 3.5×10⁶ cells/ml, 4.0×10⁶ cells/ml, 4.5×10⁶ cells/ml, 5.0×10⁶ cells/ml, 6×10⁶ cells/ml, 8×10⁶ cells/ml, or 10×10⁶ cells/ml. In some embodiments, the steady perfusion rate increases when the cells reach a set or predetermined viable cell density or concentration of, of about, or at least 0.6×10⁶ viable cells/ml, 0.8×10⁶ viable cells/ml, 1×10⁶ viable cells/ml, 1.2×10⁶ viable cells/ml, 1.4×10⁶ viable cells/ml, 1.6×10⁶ viable cells/ml, 1.8×10⁶ viable cells/ml, 2.0×10⁶ viable cells/ml, 2.5×10⁶ viable cells/ml, 3.0×10⁶ viable cells/ml, 3.5×10⁶ viable cells/ml, 4.0×10⁶ viable cells/ml, 4.5×10⁶ viable cells/ml, 5.0×10⁶ viable cells/ml, 6×10⁶ viable cells/ml, 8×10⁶ viable cells/ml, or 10×10⁶ viable cells/ml. In some embodiments, density and/or concentration of the cells or of the viable cells during the cultivation, such as under perfusion, can be determined or monitored, such as by using methods as described, including optical methods, including digital holography microscopy (DHM) or differential digital holography microscopy (DDHM).

In some embodiments, cultivation is started under conditions with no perfusion, and, perfusion is started when the density or concentration of the cells reaches a set or predetermined density or concentration. In some embodiments, the perfusion is started at a rate of, of about, or of at least 100 ml/day, 200 ml/day, 250 ml/day, 275 ml/day, 290 ml/day, 300 ml/day, 350 ml/day, 400 ml/day, 450 ml/day, 500 ml/day, 550 ml/day, 575 ml/day, 580 ml/day, 600 ml/day, 650 ml/day, 700 ml/day, 750 ml/day, 800 ml/day, 850 ml/day, 900 ml/day, 950 ml/day, 1000 ml/day, 1100 ml/day, 1160 ml/day, 1200 ml/day, 1400 ml/day, 1600 ml/day, 1800 ml/day, 2000 ml/day, 2200 ml/day, or 2400 ml/day when the density or concentration of the cells reaches a set or predetermined density or concentration. In some embodiments, the perfusion is started when the cultivated cells or cultivated viable cells reach a density or concentration of, of about, or at least 0.1×10⁶ cells/ml, 0.2×10⁶ cells/ml, 0.4×10⁶ cells/ml, 0.6×10⁶ cells/ml, 0.8×10⁶ cells/ml, 1×10⁶ cells/ml, 1.2×10⁶ cells/ml, 1.4×10⁶ cells/ml, 1.6×10⁶ cells/ml, 1.8×10⁶ cells/ml, 2.0×10⁶ cells/ml, 2.5×10⁶ cells/ml, 3.0×10⁶ cells/ml, 3.5×10⁶ cells/ml, 4.0×10⁶ cells/ml, 4.5×10⁶ cells/ml, 5.0×10⁶ cells/ml, 6×10⁶ cells/ml, 8×10⁶ cells/ml, or 10×10⁶ cells/ml.

In certain embodiments, at least part of the cultivation is performed with perfusion at a certain rate, and the perfusion rate is increased to, to about, or to at least 100 ml/day, 200 ml/day, 250 ml/day, 275 ml/day, 290 ml/day, 300 ml/day, 350 ml/day, 400 ml/day, 450 ml/day, 500 ml/day, 550 ml/day, 575 ml/day, 580 ml/day, 600 ml/day, 650 ml/day, 700 ml/day, 750 ml/day, 800 ml/day, 850 ml/day, 900 ml/day, 950 ml/day, 1000 ml/day, 1100 ml/day, 1160 ml/day, 1200 ml/day, 1400 ml/day, 1600 ml/day, 1800 ml/day, 2000 ml/day, 2200 ml/day, or 2400 ml/day when the density or concentration of the cells reaches a set or predetermined density or concentration. In some embodiments, the perfusion is started when the cultivated cells or cultivated viable cells reach a density or concentration of, of about, or at least 0.1×10⁶ cells/ml, 0.2×10⁶ cells/ml, 0.4×10⁶ cells/ml, 0.6×10⁶ cells/ml, 0.8×10⁶ cells/ml, 1×10⁶ cells/ml, 1.2×10⁶ cells/ml, 1.4×10⁶ cells/ml, 1.6×10⁶ cells/ml, 1.8×10⁶ cells/ml, 2.0×10⁶ cells/ml, 2.5×10⁶ cells/ml, 3.0×10⁶ cells/ml, 3.5×10⁶ cells/ml, 4.0×10⁶ cells/ml, 4.5×10⁶ cells/ml, 5.0×10⁶ cells/ml, 6×10⁶ cells/ml, 8×10⁶ cells/ml, or 10×10⁶ cells/ml. In some embodiments, the perfusion is performed when the cells are cultivated in a volume of, of about, or at least 300 mL, 400 mL, 500 mL, 600 mL, 700 mL, 800 mL, 900 mL, or 1000 mL. In some embodiments, the volume is 1000 mL.

In certain embodiments, cultivation is started under conditions with either no perfusion or perfusion at a certain rate, and the perfusion rate is increased to, to about, or to at 290 ml/day when the density or concentration of the cells reaches a concentration of, of about, or of at least 0.61×10⁶ cells/ml. In certain embodiments, the cells are perfused at a rate of, of about, or at least 290 ml/day when the density or concentration of the cells reaches a concentration of, of about, or of at least 0.61×10⁶ cells/ml when the cells are cultivated at a volume of, of about, or at least 1000 mL. In some embodiments, the perfusion rate is increased to, to about, or to at 580 ml/day when the density or concentration of the cells reaches a concentration of, of about, or of at least 0.81×10⁶ cells/ml. In certain embodiments, the perfusion rate is increased to, to about, or to at 1160 ml/day when the density or concentration of the cells reaches a concentration of, of about, or of at least 1.01×10⁶ cells/ml. In some embodiments, the perfusion rate is increased to, to about, or to at 1160 ml/day when the density or concentration of the cells reaches a concentration of, of about, or of at least 1.2×10⁶ cells/ml.

In aspects of the provided embodiments, the rate of perfusion, including the timing of when it is started or increased as described herein and above, is determined from assessing density and/or concentration of the cells or assessing the density and/or concentration of viable cells during the cultivation. In some embodiments, density and/or concentration of the cells can be determined using methods as described, including optical methods, including digital holography microscopy (DHM) or differential digital holography microscopy (DDHM).

In some embodiments, a composition of enriched cells, such as engineered T cells, e.g., engineered CD4+ T cells or engineered CD8+ T cells, is cultivated in the presence of a surfactant. In particular embodiments, cultivating the cells of the composition reduces the amount of shear stress that may occur during the cultivation, e.g., due to mixing, rocking, motion, and/or perfusion. In particular embodiments, the composition of enriched T cells, such as engineered T cells, e.g., engineered CD4+ T cells or engineered CD8+ T cells, is cultivated with the surfactant and at least 50%, at least 60%, at least 70%, at least 75%, at least 80%, at least 85%, at least 90%, at least 95%, at least 99%, or at least 99.9% of the T cells survive, e.g., are viable and/or do not undergo necrosis, programed cell death, or apoptosis, during or at least 1 day, 2 days, 3 days, 4 days, 5 days, 6 days, 7 days, or more than 7 days after the cultivation is complete. In particular embodiments, the composition of enriched T cells, such as engineered T cells, e.g., engineered CD4+ T cells or engineered CD8+ T cells, is cultivated in the presence of a surfactant and less than 50%, less than 40%, less than 30%, less than 25%, less than 20%, less than 15%, less than 10%, less than 5%, less than 1%, less than 0.1% or less than 0.01% of the cells undergo cell death, e.g., programmed cell death, apoptosis, and/or necrosis, such as due to shearing or shearing-induced stress.

In particular embodiments, a composition of enriched T cells, such as engineered T cells, e.g., engineered CD4+ T cells or engineered CD8+ T cells, is cultivated in the presence of between 0.1 μl/ml and 10.0 μl/ml, between 0.2 μl/ml and 2.5 μl/ml, between 0.5 μl/ml and 5 μl/ml, between 1 μl/ml and 3 μl/ml, or between 2 μl/ml and 4 μl/ml of the surfactant. In some embodiments, the composition of enriched T cells, such as engineered T cells, e.g., engineered CD4+ T cells or engineered CD8+ T cells, is cultivated in the presence of, of about, or at least 0.1 μl/ml, 0.2 μl/ml, 0.4 μl/ml, 0.6 μl/ml, 0.8 μl/ml, 1 μl/ml, 1.5 μl/ml, 2.0 μl/ml, 2.5 μl/ml, 5.0 μl/ml, 10 μl/ml, 25 μl/ml, or 50 μl/ml of the surfactant. In certain embodiments, the composition of enriched T cells is cultivated in the presence of or of about 2 μl/ml of the surfactant.

In some embodiments, a surfactant is or includes an agent that reduces the surface tension of liquids and/or solids. For example, a surfactant includes a fatty alcohol (e.g., steryl alcohol), a polyoxyethylene glycol octylphenol ether (e.g., Triton X-100), or a polyoxyethylene glycol sorbitan alkyl ester (e.g., polysorbate 20, 40, 60). In certain embodiments the surfactant is selected from the group consisting of Polysorbate 80 (PS80), polysorbate 20 (PS20), poloxamer 188 (P188). In an exemplary embodiment, the concentration of the surfactant in chemically defined feed media is about 0.0025% to about 0.25% (v/v) of PS80; about 0.0025% to about 0.25% (v/v) of PS20; or about 0.1% to about 5.0% (w/v) of P188.

In some embodiments, the surfactant is or includes an anionic surfactant, a cationic surfactant, a zwitterionic surfactant, or a nonionic surfactant added thereto. Suitable anionic surfactants include but are not limited to alkyl sulfonates, alkyl phosphates, alkyl phosphonates, potassium laurate, triethanolamine stearate, sodium lauryl sulfate, sodium dodecylsulfate, alkyl polyoxyethylene sulfates, sodium alginate, dioctyl sodium sulfosuccinate, phosphatidyl glycerol, phosphatidyl inosine, phosphatidylinositol, diphosphatidylglycerol, phosphatidylserine, phosphatidic acid and their salts, sodium carboxymethylcellulose, cholic acid and other bile acids (e.g., cholic acid, deoxycholic acid, glycocholic acid, taurocholic acid, glycodeoxycholic acid) and salts thereof (e.g., sodium deoxycholate).

In some embodiments, suitable nonionic surfactants include: glyceryl esters, polyoxyethylene fatty alcohol ethers, polyoxyethylene sorbitan fatty acid esters (polysorbates), polyoxyethylene fatty acid esters, sorbitan esters, glycerol monostearate, polyethylene glycols, polypropylene glycols, cetyl alcohol, cetostearyl alcohol, stearyl alcohol, aryl alkyl polyether alcohols, polyoxyethylene-polyoxypropylene copolymers (poloxamers), poloxamines, methylcellulose, hydroxymethyl cellulose, hydroxypropyl cellulose, hydroxypropylmethyl cellulose, noncrystalline cellulose, polysaccharides including starch and starch derivatives such as hydroxyethylstarch (HES), polyvinyl alcohol, and polyvinylpyrrolidone. In certain embodiments, the nonionic surfactant is a polyoxyethylene and polyoxypropylene copolymer and preferably a block copolymer of propylene glycol and ethylene glycol. Such polymers are sold under the tradename POLOXAMER, also sometimes referred to as PLURONIC® F68 or Kolliphor® P188. Among polyoxyethylene fatty acid esters is included those having short alkyl chains. One example of such a surfactant is SOLUTOL® HS 15, polyethylene-660-hydroxystearate.

In some embodiments, suitable cationic surfactants may include, but are not limited to, natural phospholipids, synthetic phospholipids, quaternary ammonium compounds, benzalkonium chloride, cetyltrimethyl ammonium bromide, chitosans, lauryl dimethyl benzyl ammonium chloride, acyl carnitine hydrochlorides, dimethyl dioctadecyl ammonium bromide (DDAB), dioleyoltrimethyl ammonium propane (DOTAP), dimyristoyl trimethyl ammonium propane (DMTAP), dimethyl amino ethane carbamoyl cholesterol (DC-Chol), 1,2-diacylglycero-3-(O-alkyl) phosphocholine, O-alkylphosphatidylcholine, alkyl pyridinium halides, or long-chain alkyl amines such as, for example, n-octylamine and oleylamine.

Zwitterionic surfactants are electrically neutral but possess local positive and negative charges within the same molecule. Suitable zwitterionic surfactants include but are not limited to zwitterionic phospholipids. Suitable phospholipids include phosphatidylcholine, phosphatidylethanolamine, diacyl-glycero-phosphoethanolamine (such as dimyristoyl-glycero-phosphoethanolamine (DMPE), dipalmitoyl-glycero-phosphoethanolamine (DPPE), distearoyl-glycero-phosphoethanolamine (DSPE), and dioleolyl-glycero-phosphoethanolamine (DOPE)). Mixtures of phospholipids that include anionic and zwitterionic phospholipids may be employed in this invention. Such mixtures include but are not limited to lysophospholipids, egg or soybean phospholipid or any combination thereof. The phospholipid, whether anionic, zwitterionic or a mixture of phospholipids, may be salted or desalted, hydrogenated or partially hydrogenated or natural semi-synthetic or synthetic.

In certain embodiments, the surfactant is poloxamer, e.g., poloxamer 188. In some embodiments, a composition of enriched T cells is cultivated in the presence of between 0.1 μl/ml and 10.0 μl/ml, between 0.2 μl/ml and 2.5 μl/ml, between 0.5 μl/ml and 5 μl/ml, between 1 μl/ml and 3 μl/ml, or between 2 μl/ml and 4 μl/ml of poloxamer. In some embodiments, the composition of enriched T cells is cultivated in the presence of, of about, or at least 0.1 μl/ml, 0.2 μl/ml, 0.4 μl/ml, 0.6 μl/ml, 0.8 μl/ml, 1 μl/ml, 1.5 μl/ml, 2.0 μl/ml, 2.5 μl/ml, 5.0 μl/ml, 10 μl/ml, 25 μl/ml, or 50 μl/ml of the surfactant. In certain embodiments, the composition of enriched T cells is cultivated in the presence of or of about 2 μl/ml of poloxamer.

In particular embodiments, the cultivation ends, such as by harvesting cells, when cells achieve a threshold amount, concentration, and/or expansion. In particular embodiments, the cultivation ends when the cell achieve or achieve about or at least a 1.5-fold expansion, a 2-fold expansion, a 2.5-fold expansion, a 3-fold expansion, a 3.5-fold expansion, a 4-fold expansion, a 4.5-fold expansion, a 5-fold expansion, a 6-fold expansion, a 7-fold expansion, a 8-fold expansion, a 9-fold expansion, a 10-fold expansion, or greater than a 10-fold expansion, e.g., with respect and/or in relation to the amount of density of the cells at the start or initiation of the cultivation. In some embodiments, the threshold expansion is a 4-fold expansion, e.g., with respect and/or in relation to the amount of density of the cells at the start or initiation of the cultivation.

In some embodiments, the cultivation ends, such as by harvesting cells, when the cells achieve a threshold total amount of cells, e.g., threshold cell count. In some embodiments, the cultivation ends when the cells achieve a threshold total nucleated cell (TNC) count. In some embodiments, the cultivation ends when the cells achieve a threshold viable amount of cells, e.g., threshold viable cell count. In some embodiments, the threshold cell count is or is about or is at least of 50×10⁶ cells, 100×10⁶ cells, 200×10⁶ cells, 300×10⁶ cells, 400×10⁶ cells, 600×10⁶ cells, 800×10⁶ cells, 1000×10⁶ cells, 1200×10⁶ cells, 1400×10⁶ cells, 1600×10⁶ cells, 1800×10⁶ cells, 2000×10⁶ cells, 2500×10⁶ cells, 3000×10⁶ cells, 4000×10⁶ cells, 5000×10⁶ cells, 10,000×10⁶ cells, 12,000×10⁶ cells, 15,000×10⁶ cells or 20,000×10⁶ cells, or any of the foregoing threshold of viable cells. In particular embodiments, the cultivation ends when the cells achieve a threshold cell count. In some embodiments, the cultivation ends at, at about, or within 6 hours, 12 hours, 24 hours, 36 hours, 1 day, 2 days, 3 days, 4 days, 5 days, 6 days, or 7 or more days, after the threshold cell count is achieved. In particular embodiments, the cultivation is ended at or about 1 day after the threshold cell count is achieved. In certain embodiments, the threshold density is, is about, or is at least 0.1×10⁶ cells/ml, 0.5×10⁶ cells/ml, 1×10⁶ cells/ml, 1.2×10⁶ cells/ml, 1.5×10⁶ cells/ml, 1.6×10⁶ cells/ml, 1.8×10⁶ cells/ml, 2.0×10⁶ cells/ml, 2.5×10⁶ cells/ml, 3.0×10⁶ cells/ml, 3.5×10⁶ cells/ml, 4.0×10⁶ cells/ml, 4.5×10⁶ cells/ml, 5.0×10⁶ cells/ml, 6×10⁶ cells/ml, 8×10⁶ cells/ml, or 10×10⁶ cells/ml, or any of the foregoing threshold of viable cells. In particular embodiments, the cultivation ends when the cells achieve a threshold density. In some embodiments, the cultivation ends at, at about, or within 6 hours, 12 hours, 24 hours, 36 hours, 1 day, 2 days, 3 days, 4 days, 5 days, 6 days, or 7 or more days, after the threshold density is achieved. In particular embodiments, the cultivation is ended at or about 1 day after the threshold density is achieved.

In some embodiments, the cultivation step is performed for the amount of time required for the cells to achieve a threshold amount, density, and/or expansion. In some embodiments, the cultivation is performed for or for about, or for less than, 6 hours, 12 hours, 18 hours, 24 hours, 36 hours, 48 hours, 60 hours, 72 hours, 2 days, 3 days 4 days, 5 days, 6 days, 7 days, 7 days, 8 days, 9 days, 10 days, 1 week, 2 weeks, 3 weeks, or 4 weeks. In particular embodiments, the mean amount of time required for the cells of a plurality of separate compositions of enriched T cells that were isolated, enriched, and/or selected from different biological samples to achieve the threshold density is, is about, or is less than 6 hours, 12 hours, 18 hours, 24 hours, 36 hours, 48 hours, 60 hours, 72 hours, 2 days, 3 days 4 days, 5 days, 6 days, 7 days, 7 days, 8 days, 9 days, 10 days, 1 week, 2 weeks, 3 weeks, or 4 weeks. In certain embodiments, the mean amount of time required for the cells of a plurality of separate compositions of enriched T cells that were isolated, enriched, and/or selected from different biological samples to achieve the threshold density is, is about, or is less than 6 hours, 12 hours, 18 hours, 24 hours, 36 hours, 48 hours, 60 hours, 72 hours, 2 days, 3 days 4 days, 5 days, 6 days, 7 days, 7 days, 8 days, 9 days, 10 days, 1 week, 2 weeks, 3 weeks, or 4 weeks.

In certain embodiments, the cultivation step is performed for a minimum of 4 days, 5 days, 6 days, 7 days, 7 days, 8 days, 9 days, or 10 days, and/or until 12 hours, 24 hours, 36 hours, 1 day, 2 days, or 3 days after the cells active a threshold cell count (or number) or threshold viable cell count (or number) of or of about 1000×10⁶ cells, 1200×10⁶ cells, 1400×10⁶ cells, 1600×10⁶ cells, 1800×10⁶ cells, 2000×10⁶ cells, 2500×10⁶ cells, 3000×10⁶ cells, 4000×10⁶ cells, or 5000×10⁶ cells. In some embodiments, the cultivation step is performed until 1 day after the cells achieve a threshold cell count of or of about 1200×10⁶ cells and are cultured for a minimum of 10 days, and/or until 1 day after the cells achieve a threshold cell count of or of about 5000×10⁶ cells. In some embodiments, the cultivation step is performed until 1 day after the cells achieve a threshold cell count of or of about 1200×10⁶ cells and are cultured for a minimum of 9 days, and/or until 1 day after the cells achieve a threshold cell count of or of about 5000×10⁶ cells. In some embodiments, the cultivation step is performed until 1 day after the cells achieve a threshold cell count of or of about 1000×10⁶ cells and are cultured for a minimum of 8 days, and/or until 1 day after the cells achieve a threshold cell count of or of about 4000×10⁶ cells. In certain embodiments, the cultivation is an expansion step and is performed for a minimum of 4 days, 5 days, 6 days, 7 days, 7 days, 8 days, 9 days, or 10 days, and/or until 12 hours, 24 hours, 36 hours, 1 day, 2 days, or 3 days after the cells active a threshold cell count (or number) or threshold viable cell count (or number) of or of about 1000×10⁶ cells, 1200×10⁶ cells, 1400×10⁶ cells, 1600×10⁶ cells, 1800×10⁶ cells, 2000×10⁶ cells, 2500×10⁶ cells, 3000×10⁶ cells, 4000×10⁶ cells, or 5000×10⁶ cells. In some embodiments, the expansion step is performed until 1 day after the cells achieve a threshold cell count of or of about 1200×10⁶ cells and are expanded for a minimum of 10 days, and/or until 1 day after the cells achieve a threshold cell count of or of about 5000×10⁶ cells. In some embodiments, the expansion step is performed until 1 day after the cells achieve a threshold cell count of or of about 1200×10⁶ cells and are expanded for a minimum of 9 days, and/or until 1 day after the cells achieve a threshold cell count of or of about 5000×10⁶ cells. In some embodiments, the expansion step is performed until 1 day after the cells achieve a threshold cell count of or of about 1000×10⁶ cells and are expanded for a minimum of 8 days, and/or until 1 day after the cells achieve a threshold cell count of or of about 4000×10⁶ cells. In some embodiments, the expansion step is performed until 1 day after the cells achieve a threshold cell count of or of about 1400×10⁶ cells and are expanded for a minimum of 5 days, and/or until 1 day after the cells achieve a threshold cell count of or of about 4000×10⁶ cells.

In some embodiments, the cultivation is performed for at least a minimum amount of time. In some embodiments, the cultivation is performed for at least 14 days, at least 12 days, at least 10 days, at least 7 days, at least 6 days, at least 5 days, at least 4 days, at least 3 days, at least 2 days, at least 36 hours, at least 24 hours, at least 12 hours, or at least 6 hours, even if the threshold is achieved prior to the minimum amount of time. In some embodiments, increasing the minimum amount of time that the cultivation is performed, may, in some cases, reduce the activation and/or reduce the level or one or more activation markers, in the cultivated cells, formulated cells, and/or cells of the output composition. In some embodiments, the minimum cultivation time counts from a determined point an exemplary process (e.g., a selection step; a thaw step; and/or an activation step) to the day the cells are harvested.

In aspects of the provided embodiments, the density and/or concentration of the cells or of the viable cells during the cultivation is monitored or carried out during the cultivation, such as until a threshold amount, density, and/or expansion is achieved as described. In some embodiments such methods include those as described, including optical methods, including digital holography microscopy (DHM) or differential digital holography microscopy (DDHM).

In certain embodiments, the cultivated cells are output cells. In some embodiments, a composition of enriched T cells, such as engineered T cells, that has been cultivated is an output composition of enriched T cells. In particular embodiments, CD4+ T cells and/or CD8+ T cells that have been cultivated are output CD4+ and/or CD8+ T cells. In particular embodiments, a composition of enriched CD4+ T cells, such as engineered CD4+ T cells, that has been cultivated is an output composition of enriched CD4+ T cells. In some embodiments, a composition of enriched CD8+ T cells, such as engineered CD8+ T cells, that has been cultivated is an output composition of enriched CD8+ T cells.

In some embodiments, the cells are cultivated under conditions that promote proliferation and/or expansion in presence of one or more cytokines. In particular embodiments, at least a portion of the cultivation is performed with constant mixing and/or perfusion, such as mixing or perfusion controlled by a bioreactor. In some embodiments, the cells are cultivated in the presence or one or more cytokines and with a surfactant, e.g., poloxamer, such as poloxamer 188, to reduce shearing and/or shear stress from constant mixing and/or perfusion. In some embodiments, a composition of enriched CD4+ T cells, such as engineered CD4+ T cells, is cultivated in the presence of recombinant IL-2, IL-7, IL-15, and poloxamer, wherein at least a portion of the cultivating is performed with constant mixing and/or perfusion. In certain embodiments, a composition of enriched CD8+ T cells, such as engineered CD8+ T cells, is cultivated in the presence of recombinant IL-2, IL-15, and poloxamer, wherein at least a portion of the cultivating is performed with constant mixing and/or perfusion. In some embodiments, the cultivation is performed until the cells reach as threshold expansion of at least 4-fold e.g., as compared to the start of the cultivation.

### 1. Monitoring Cells During Cultivation

In some embodiments, the cells are monitored during the cultivation step. Monitoring may be performed, for example, to ascertain (e.g., measure, quantify) cell morphology, cell viability, cell death, and/or cell concentration (e.g., viable cell concentration). In some embodiments, the monitoring is performed manually, such as by a human operator. In some embodiments, the monitoring is performed by an automated system. The automated system may require minimal or no manual input to monitor the cultivated cells. In some embodiments, the monitoring is performed both manually and by an automated system.

In certain embodiments, the cells are monitored by an automated system requiring no manual input. In some embodiments, the automated system is compatible with a bioreactor, for example a bioreactor as described herein, such that cells undergoing cultivation can be removed from the bioreactor, monitored, and subsequently returned to the bioreactor. In some embodiments, the monitoring and cultivation occur in a closed loop configuration. In some aspects, in a closed loop configuration, the automated system and bioreactor remain sterile. In embodiments, the automated system is sterile. In some embodiments, the automated system is an in-line system.

In some embodiments, the automated system includes the use of optical techniques (e.g., microscopy) for detecting cell morphology, cell viability, cell death, and/or cell concentration (e.g., viable cell concentration). Any optical technique suitable for determining, for example, cell features, viability, and concentration are contemplated herein. Non-limiting examples of useful optical techniques include bright field microscopy, fluorescence microscopy, differential interference contrast (DIC) microscopy, phase contrast microscopy, digital holography microscopy (DHM), differential digital holography microscopy (DDHM), or a combination thereof. Differential digital holography microscopy, DDHM, and differential DHM may be used herein interchangeably. In certain embodiments, the automated system includes a differential digital holography microscope. In certain embodiments, the automated system includes a differential digital holography microscope including illumination means (e.g., laser, led). Descriptions of DDHM methodology and use may be found, for example, in U.S. Pat. No. 7,362,449; EP 1,631,788; U.S. Pat. Nos. 9,904,248; and 9,684,281, which are incorporated herein by reference in their entirety.

DDHM permits label-free, non-destructive imaging of cells, resulting in high-contrast holographic images. The images may undergo object segmentation and further analysis to obtain a plurality of morphological features that quantitatively describe the imaged objects (e.g., cultivated cells, cellular debris). As such, various features (e.g., cell morphology, cell viability, cell concentration) may be directly assessed or calculated from DDHM using, for example, the steps of image acquisition, image processing, image segmentation, and feature extraction. In some embodiments, the automated system includes a digital recording device to record holographic images. In some embodiments, the automated system includes a computer including algorithms for analyzing holographic images. In some embodiments, the automated system includes a monitor and/or computer for displaying the results of the holographic image analysis. In some embodiments, the analysis is automated (i.e., capable of being performed in the absence of user input). An example of a suitable automated system for monitoring cells during the cultivating step includes, but is not limited to, Ovizio iLine F (Ovizio Imaging Systems NV/SA, Brussels, Belgium).

In certain embodiments, the monitoring is performed continuously during the cultivation step. In some embodiments, the monitoring is performed in real-time during the cultivation step. In some embodiments, the monitoring is performed at discrete time points during the cultivation step. In some embodiments, the monitoring is performed at least every 15 minutes for the duration of the cultivating step. In some embodiments, the monitoring is performed at least every 30 minutes for the duration of the cultivating step. In some embodiments, the monitoring is performed at least every 45 minutes for the duration of the cultivating step. In some embodiments, the monitoring is performed at least every hour for the duration of the cultivating step. In some embodiments, the monitoring is performed at least every 2 hours for the duration of the cultivating step. In some embodiments, the monitoring is performed at least every 4 hours for the duration of the cultivating step. In some embodiments, the monitoring is performed at least every 6 hours for the duration of the cultivating step. In some embodiments, the monitoring is performed at least every 8 hours for the duration of the cultivating step. In some embodiments, the monitoring is performed at least every 10 hours for the duration of the cultivating step. In some embodiments, the monitoring is performed at least every 12 hours for the duration of the cultivating step. In some embodiments, the monitoring is performed at least every 14 hours for the duration of the cultivating step. In some embodiments, the monitoring is performed at least every 16 hours for the duration of the cultivating step. In some embodiments, the monitoring is performed at least every 18 hours for the duration of the cultivating step. In some embodiments, the monitoring is performed at least every 20 hours for the duration of the cultivating step. In some embodiments, the monitoring is performed at least every 22 hours for the duration of the cultivating step. In some embodiments, the monitoring is performed at least once a day for the duration of the cultivating step. In some embodiments, the monitoring is performed at least once every second day for the duration of the cultivating step. In some embodiments, the monitoring is performed at least once every third day for the duration of the cultivating step. In some embodiments, the monitoring is performed at least once every fourth day for the duration of the cultivating step. In some embodiments, the monitoring is performed at least once every fifth day for the duration of the cultivating step. In some embodiments, the monitoring is performed at least once every sixth day for the duration of the cultivating step. In some embodiments, the monitoring is performed at least once every seventh day for the duration of the cultivating step. In some embodiments, the monitoring is performed at least once every eighth day for the duration of the cultivating step. In some embodiments, the monitoring is performed at least once every ninth day for the duration of the cultivating step. In some embodiments, the monitoring is performed at least once every tenth day for the duration of the cultivating step. In some embodiments, the monitoring is performed at least once during the cultivating step.

In some embodiments, features of the cells that can be determined by the monitoring, including using optical techniques such as DHM or DDHM, include cell viability, cell concentration, cell number and/or cell density. In some embodiments, cell viability is characterized or determined. In some embodiments, cell concentration, density and/or number is characterized or determined. In some embodiments, viable cell concentration, viable cell number and/or viable cell density is characterized or determined. In some embodiments, the cultivated cells are monitored by the automated system until a threshold of expansion is reached, such as described above. In some embodiments, once a threshold of expansion is reached, the cultivated cells are harvested, such as by automatic or manual methods, for example, by a human operator. The threshold of expansion may depend on the total concentration, density and/or number of cultured cells determined by the automated system. Alternatively, the threshold of expansion may depend on the viable cell concentration, density and/or number.

In some embodiments, the harvested cells are formulated as described, such as in the presence of a pharmaceutically acceptable carrier. In some embodiments, the harvested cells are formulated in the presence of a cryoprotectant.

### E. Agents

In various embodiments, a method for manufacturing T cells is provided that expands undifferentiated or developmentally potent T cells comprising contacting T cells with an agent that modulates a PI3K pathway in the cells. In various embodiments, a method for manufacturing T cells is provided that expands undifferentiated or developmentally potent T cells comprising contacting T cells with an agent that modulates a PI3K/AKT/mTOR pathway in the cells. The cells may be contacted prior to, during, and/or after activation and expansion. The T cell compositions retain sufficient T cell potency such that they may undergo multiple rounds of expansion without a substantial increase in differentiation.

As used herein, the terms “modulate,” “modulator,” or “modulatory agent” or comparable term refer to an agent's ability to elicit a change, e.g., in a cell signaling pathway. A modulator may increase or decrease an amount, activity of a pathway component or increase or decrease a desired effect or output of a cell signaling pathway. In one embodiment, the modulator is an inhibitor. In certain embodiments, the modulator is an activator.

An “agent” for use in manufacturing T cells can be a compound, small molecule, e.g., a small organic molecule, nucleic acid, polypeptide, or a fragment, isoform, variant, analog, or derivative thereof used in the modulation of a PI3K/AKT/mTOR pathway.

A “small molecule” refers to a composition that has a molecular weight of less than about 5 kD, less than about 4 kD, less than about 3 kD, less than about 2 kD, less than about 1 kD, or less than about 0.5 kD. Small molecules may comprise nucleic acids, peptides, polypeptides, peptidomimetics, peptoids, carbohydrates, lipids, components thereof or other organic or inorganic molecules. Libraries of chemical and/or biological mixtures, such as fungal, bacterial, or algal extracts, are known in the art and can be screened with any of the assays of the present disclosure. Methods for the synthesis of molecular libraries are known in the art (see, e.g., Carell et al., 1994a; Carell et al., 1994b; Cho et al., 1993; DeWitt et al., 1993; Gallop et al., 1994; Zuckermann et al., 1994).

An “analog” refers to a small organic compound, a nucleotide, a protein, or a polypeptide that possesses similar or identical activity or function(s) as the compound, nucleotide, protein or polypeptide or compound having the desired activity of the present disclosure, but need not necessarily comprise a sequence or structure that is similar or identical to the sequence or structure of a preferred embodiment.

A “derivative” refers to either a compound, a protein or polypeptide that comprises an amino acid sequence of a parent protein or polypeptide that has been altered by the introduction of amino acid residue substitutions, deletions or additions, or a nucleic acid or nucleotide that has been modified by either introduction of nucleotide substitutions or deletions, additions or mutations. The derivative nucleic acid, nucleotide, protein or polypeptide possesses a similar or identical function as the parent polypeptide.

In various embodiments, the agent that modulates a PI3K pathway activates a component of the pathway. In this context, an “activator,” or “agonist” refers to an agent that promotes, increases, or induces one or more activities of a molecule in a PI3K/AKT/mTOR pathway including, without limitation, a molecule that inhibits one or more activities of a PI3K.

In various embodiments, the agent that modulates a PI3K pathway inhibits a component of the pathway. In this context, an “inhibitor” or “antagonist” refers to an agent that inhibits, decreases, or reduces one or more activities of a molecule in a PI3K pathway including, without limitation, a PI3K. In one embodiment, the inhibitor is a dual molecule inhibitor. In particular embodiment, the inhibitor may inhibit a class of molecules have the same or substantially similar activities (a pan-inhibitor) or may specifically inhibit a molecule's activity (a selective or specific inhibitor). Inhibition may also be irreversible or reversible.

In one embodiment, the inhibitor has an IC50 of at least 1 nM, at least 2 nM, at least 5 nM, at least 10 nM, at least 50 nM, at least 100 nM, at least 200 nM, at least 500 nM, at least 1 μM, at least 10 μM, at least 50 μM, or at least 100 μM. IC50 determinations can be accomplished using any conventional techniques known in the art. For example, an IC50 can be determined by measuring the activity of a given enzyme in the presence of a range of concentrations of the inhibitor under study. The experimentally obtained values of enzyme activity then are plotted against the inhibitor concentrations used. The concentration of the inhibitor that shows 50% enzyme activity (as compared to the activity in the absence of any inhibitor) is taken as the “IC50” value. Analogously, other inhibitory concentrations can be defined through appropriate determinations of activity.

In various embodiments, T cells are contacted or treated or cultured with one or more modulators of a PI3K pathway at a concentration of at least 1 nM, at least 2 nM, at least 5 nM, at least 10 nM, at least 50 nM, at least 100 nM, at least 200 nM, at least 500 nM, at least 1 μM, at least 10 μM, at least 50 μM, at least 100 μM, or at least 1 M.

In particular embodiments, T cells may be contacted or treated or cultured with one or more modulators of a PI3K pathway for at least 12 hours, 18 hours, at least 1, 2, 3, 4, 5, 6, or 7 days, at least 2 weeks, at least 1, 2, 3, 4, 5, or 6 months or more with 1, 2, 3, 4, 5, 6, 7, 8, 9, or 10 or more rounds of expansion.

### 1. PI3K/Akt/mTOR Pathway

The phosphatidyl-inositol-3 kinase/Akt/mammalian target of rapamycin pathway serves as a conduit to integrate growth factor signaling with cellular proliferation, differentiation, metabolism, and survival. PI3Ks are a family of highly conserved intracellular lipid kinases. Class IA PI3Ks are activated by growth factor receptor tyrosine kinases (RTKs), either directly or through interaction with the insulin receptor substrate family of adaptor molecules. This activity results in the production of phosphatidyl-inositol-3,4,5-trisphospate (PIP3) a regulator of the serine/threonine kinase Akt. mTOR acts through the canonical PI3K pathway via 2 distinct complexes, each characterized by different binding partners that confer distinct activities. mTORC1 (mTOR in complex with PRAS40, raptor, and mLST8/GbL) acts as a downstream effector of PI3K/Akt signaling, linking growth factor signals with protein translation, cell growth, proliferation, and survival. mTORC2 (mTOR in complex with rictor, mSIN1, protor, and mLST8) acts as an upstream activator of Akt.

Upon growth factor receptor-mediated activation of PI3K, Akt is recruited to the membrane through the interaction of its pleckstrin homology domain with PIP3, thus exposing its activation loop and enabling phosphorylation at threonine 308 (Thr308) by the constitutively active phosphoinositide-dependent protein kinase 1 (PDK1). For maximal activation, Akt is also phosphorylated by mTORC2, at serine 473 (Ser473) of its C-terminal hydrophobic motif. DNA-PK and HSP have also been shown to be important in the regulation of Akt activity. Akt activates mTORC1 through inhibitory phosphorylation of TSC2, which along with TSC1, negatively regulates mTORC1 by inhibiting the Rheb GTPase, a positive regulator of mTORC1. mTORC1 has 2 well-defined substrates, p70S6K (referred to hereafter as S6K1) and 4E-BP1, both of which critically regulate protein synthesis. Thus, mTORC1 is an important downstream effector of PI3K, linking growth factor signaling with protein translation and cellular proliferation.

### 2. PI3K Inhibitors

As used herein, the term “PI3K inhibitor” refers to a nucleic acid, peptide, compound, or small organic molecule that binds to and inhibits at least one activity of PI3K. The PI3K proteins can be divided into three classes, class 1 PI3Ks, class 2 PI3Ks, and class 3 PI3Ks. Class 1 PI3Ks exist as heterodimers consisting of one of four p110 catalytic subunits (p 110α, p110β, p110δ, and p110γ) and one of two families of regulatory subunits. In a particular embodiment, a PI3K inhibitor of the present disclosure targets the class 1 PI3K inhibitors. In one embodiment, a PI3K inhibitor will display selectivity for one or more isoforms of the class 1 PI3K inhibitors (i.e., selectivity for p110α, p110β, p110δ, and p110γ or one or more of p110α, p110β, p110δ, and p110γ). In another aspect, a PI3K inhibitor will not display isoform selectivity and be considered a “pan-PI3K inhibitor.” In one embodiment, a PI3K inhibitor will compete for binding with ATP to the PI3K catalytic domain.

In certain embodiments, a PI3K inhibitor can, for example, target PI3K as well as additional proteins in the PI3K-AKT-mTOR pathway. In particular embodiments, a PI3K inhibitor that targets both mTOR and PI3K can be referred to as either an mTOR inhibitor or a PI3K inhibitor. A PI3K inhibitor that only targets PI3K can be referred to as a selective PI3K inhibitor. In one embodiment, a selective PI3K inhibitor can be understood to refer to an agent that exhibits a 50% inhibitory concentration with respect to PI3K that is at least 10-fold, at least 20-fold, at least 30-fold, at least 50-fold, at least 100-fold, at least 1000-fold, or more, lower than the inhibitor's IC50 with respect to mTOR and/or other proteins in the pathway.

In a particular embodiment, exemplary PI3K inhibitors inhibit PI3K with an IC50 (concentration that inhibits 50% of the activity) of about 200 nM or less, preferably about 100 nm or less, even more preferably about 60 nM or less, about 25 nM, about 10 nM, about 5 nM, about 1 nM, 100 M, 50 M, 25 M, 10 M, 1 M, or less. In one embodiment, a PI3K inhibitor inhibits PI3K with an IC50 from about 2 nM to about 100 nm, more preferably from about 2 nM to about 50 nM, even more preferably from about 2 nM to about 15 nM.

Illustrative examples of PI3K inhibitors suitable for use in the T cell manufacturing methods contemplated herein include, but are not limited to, BKM120 (class 1 PI3K inhibitor, Novartis), XL147 (class 1 PI3K inhibitor, Exelixis), (pan-PI3K inhibitor, GlaxoSmithKline), and PX-866 (class 1 PI3K inhibitor; p110α, p110β, and p110γ isoforms, Oncothyreon).

Other illustrative examples of selective PI3K inhibitors include, but are not limited to BYL719, GSK2636771, TGX-221, AS25242, CAL-101, ZSTK474, and IPI-145.

Further illustrative examples of pan-PI3K inhibitors include, but are not limited to BEZ235, LY294002, GSK1059615, TG100713, and GDC-0941.

### 3. AKT Inhibitors

As used herein, the term “AKT inhibitor” refers to a nucleic acid, peptide, compound, or small organic molecule that inhibits at least one activity of AKT. AKT inhibitors can be grouped into several classes, including lipid-based inhibitors (e.g., inhibitors that target the pleckstrin homology domain of AKT which prevents AKT from localizing to plasma membranes), ATP-competitive inhibitors, and allosteric inhibitors. In one embodiment, AKT inhibitors act by binding to the AKT catalytic site. In a particular embodiment, Akt inhibitors act by inhibiting phosphorylation of downstream AKT targets such as mTOR. In certain embodiments, AKT activity is inhibited by inhibiting the input signals to activate Akt by inhibiting, for example, DNA-PK activation of AKT, PDK-1 activation of AKT, and/or mTORC2 activation of Akt.

AKT inhibitors can target all three AKT isoforms, AKT1, AKT2, AKT3 or may be isoform selective and target only one or two of the AKT isoforms. In one embodiment, an AKT inhibitor can target AKT as well as additional proteins in the PI3K-AKT-mTOR pathway. An AKT inhibitor that only targets AKT can be referred to as a selective AKT inhibitor. In one embodiment, a selective AKT inhibitor can be understood to refer to an agent that exhibits a 50% inhibitory concentration with respect to AKT that is at least 10-fold, at least 20-fold, at least 30-fold, at least 50-fold, at least 100-fold, at least 1000-fold, or more lower than the inhibitor's IC50 with respect to other proteins in the pathway.

In a particular embodiment, exemplary AKT inhibitors inhibit AKT with an IC50 (concentration that inhibits 50% of the activity) of about 200 nM or less, preferably about 100 nm or less, even more preferably about 60 nM or less, about 25 nM, about 10 nM, about 5 nM, about 1 nM, 100 μM, 50 μM, 25 μM, 10 μM, 1 μM, or less. In one embodiment, an AKT inhibits AKT with an IC50 from about 2 nM to about 100 nm, more preferably from about 2 nM to about 50 nM, even more preferably from about 2 nM to about 15 nM.

Illustrative examples of AKT inhibitors for use in combination with auristatin based antibody-drug conjugates include, for example, perifosine (Keryx), MK2206 (Merck), VQD-002 (VioQuest), XL418 (Exelixis), GSK690693, GDC-0068, and PX316 (PROLX Pharmaceuticals).

An illustrative, non-limiting example of a selective Akt1 inhibitor is A-674563.

An illustrative, non-limiting example of a selective Akt2 inhibitor is CCT128930.

In particular embodiments, the Akt inhibitor DNA-PK activation of Akt, PDK-1 activation of Akt, mTORC2 activation of Akt, or HSP activation of Akt.

Illustrative examples of DNA-PK inhibitors include, but are not limited to, NU7441, PI-103, NU7026, PIK-75, and PP-121.

4. mTOR Inhibitors

The terms “mTOR inhibitor” or “agent that inhibits mTOR” refers to a nucleic acid, peptide, compound, or small organic molecule that inhibits at least one activity of an mTOR protein, such as, for example, the serine/threonine protein kinase activity on at least one of its substrates (e.g., p70S6 kinase 1, 4E-BP1, AKT/PKB and eEF2). mTOR inhibitors are able to bind directly to and inhibit mTORC1, mTORC2 or both mTORC1 and mTORC2.

Inhibition of mTORC1 and/or mTORC2 activity can be determined by a reduction in signal transduction of the PI3K/Akt/mTOR pathway. A wide variety of readouts can be utilized to establish a reduction of the output of such signaling pathway. Some non-limiting exemplary readouts include (1) a decrease in phosphorylation of Akt at residues, including but not limited to 5473 and T308; (2) a decrease in activation of Akt as evidenced, for example, by a reduction of phosphorylation of Akt substrates including but not limited to Fox01/03a T24/32, GSK3a/a; S21/9, and TSC2 T1462; (3) a decrease in phosphorylation of signaling molecules downstream of mTOR, including but not limited to ribosomal S6 S240/244, 70S6K T389, and 4EBP1 T37/46; and (4) inhibition of proliferation of cancerous cells.

In one embodiment, the mTOR inhibitors are active site inhibitors. These are mTOR inhibitors that bind to the ATP binding site (also referred to as ATP binding pocket) of mTOR and inhibit the catalytic activity of both mTORC1 and mTORC2. One class of active site inhibitors suitable for use in the T cell manufacturing methods contemplated herein are dual specificity inhibitors that target and directly inhibit both PI3K and mTOR. Dual specificity inhibitors bind to both the ATP binding site of mTOR and PI3K. Illustrative examples of such inhibitors include, but are not limited to: imidazoquinazolines, wortmannin, LY294002, PI-103 (Cayman Chemical), SF1126 (Semafore), BGT226 (Novartis), XL765 (Exelixis) and NVP-BEZ235 (Novartis).

Another class of mTOR active site inhibitors suitable for use in the methods contemplated herein selectively inhibit mTORC1 and mTORC2 activity relative to one or more type I phosphatidylinositol 3-kinases, e.g., PI3 kinase α, β, γ, or δ. These active site inhibitors bind to the active site of mTOR but not PI3K. Illustrative examples of such inhibitors include, but are not limited to: pyrazolopyrimidines, Torin1 (Guertin and Sabatini), PP242 (2-(4-Amino-1-isopropyl-1H-pyrazolo[3,4-d]pyrimidin-3-yl)-1H-indol-5-ol), PP30, Ku-0063794, WAY-600 (Wyeth), WAY-687 (Wyeth), WAY-354 (Wyeth), and AZD8055 (Liu et al., Nature Review, 8, 627-644, 2009).

In one embodiment, a selective mTOR inhibitor refers to an agent that exhibits a 50% inhibitory concentration (IC50) with respect to mTORC1 and/or mTORC2, that is at least 10-fold, at least 20-fold, at least 50-fold, at least 100-fold, at least 1000-fold, or more, lower than the inhibitor's IC50 with respect to one, two, three, or more type I PI3-kinases or to all of the type I PI3-kinases. Another class of mTOR inhibitors for use in the present disclosure is referred to herein as “rapalogs.” As used herein the term “rapalogs” refers to compounds that specifically bind to the mTOR FRB domain (FKBP rapamycin binding domain), are structurally related to rapamycin, and retain the mTOR inhibiting properties. The term rapalogs excludes rapamycin. Rapalogs include esters, ethers, oximes, hydrazones, and hydroxylamines of rapamycin, as well as compounds in which functional groups on the rapamycin core structure have been modified, for example, by reduction or oxidation. Pharmaceutically acceptable salts of such compounds are also considered to be rapamycin derivatives. Illustrative examples of rapalogs suitable for use in the methods contemplated herein include, without limitation, temsirolimus (CC1779), everolimus (RAD001), deforolimus (AP23573), AZD8055 (AstraZeneca), and OSI-027 (OSI).

In one embodiment, the agent is the mTOR inhibitor rapamycin (sirolimus).

In a particular embodiment, exemplary mTOR inhibitors for use herein inhibit either mTORC1, mTORC2 or both mTORC1 and mTORC2 with an IC50 (concentration that inhibits 50% of the activity) of about 200 nM or less, preferably about 100 nm or less, even more preferably about 60 nM or less, about 25 nM, about 10 nM, about 5 nM, about 1 nM, 100 μM, 50 μM, 25 μM, 10 μM, 1 μM, or less. In one aspect, a mTOR inhibitor for use herein inhibits either mTORC1, mTORC2 or both mTORC1 and mTORC2 with an IC50 from about 2 nM to about 100 nm, more preferably from about 2 nM to about 50 nM, even more preferably from about 2 nM to about 15 nM.

In one embodiment, exemplary mTOR inhibitors inhibit either PI3K and mTORC1 or mTORC2 or both mTORC1 and mTORC2 and PI3K with an IC50 (concentration that inhibits 50% of the activity) of about 200 nM or less, preferably about 100 nm or less, even more preferably about 60 nM or less, about 25 nM, about 10 nM, about 5 nM, about 1 nM, 100 μM, 50 μM, 25 μM, 10 μM, 1 μM, or less. In one aspect, a mTOR inhibitor for use herein inhibits PI3K and mTORC1 or mTORC2 or both mTORC1 and mTORC2 and PI3K with an IC50 from about 2 nM to about 100 nm, more preferably from about 2 nM to about 50 nM, even more preferably from about 2 nM to about 15 nM.

Further illustrative examples of mTOR inhibitors suitable for use in particular embodiments contemplated herein include, but are not limited to AZD8055, INK128, rapamycin, PF-04691502, and everolimus.

mTOR has been shown to demonstrate a robust and specific catalytic activity toward the physiological substrate proteins, p70 S6 ribosomal protein kinase I (p70S6K1) and eIF4E binding protein 1 (4EBP1) as measured by phosphor-specific antibodies in Western blotting.

In one embodiment, the inhibitor of the PI3K/AKT/mTOR pathway is an s6 kinase inhibitor selected from the group consisting of: BI-D1870, H89, PF-4708671, FMK, and AT7867.

### F. Formulating the Cells

In some embodiments, the provided methods for manufacturing, generating or producing a cell therapy and/or engineered cells may include formulation of cells, such as formulation of genetically engineered cells resulting from the provided processing steps prior to or after the incubating, engineering, and cultivating, and/or one or more other processing steps as described. In some embodiments, the provided methods associated with formulation of cells include processing transduced cells, such as cells transduced and/or expanded using the processing steps described above, in a closed system. In some embodiments, the dose of cells comprising cells engineered with a recombinant antigen receptor, e.g., CAR or TCR, is provided as a composition or formulation, such as a pharmaceutical composition or formulation. Such compositions can be used in accord with the provided methods, such as in the prevention or treatment of diseases, conditions, and disorders, or in detection, diagnostic, and prognostic methods.

In some cases, the cells are processed in one or more steps (e.g., carried out in the centrifugal chamber and/or closed system) for manufacturing, generating or producing a cell therapy and/or engineered cells may include formulation of cells, such as formulation of genetically engineered cells resulting from the provided transduction processing steps prior to or after the culturing, e.g., cultivation and expansion, and/or one or more other processing steps as described. In some cases, the cells can be formulated in an amount for dosage administration, such as for a single unit dosage administration or multiple dosage administration. In some embodiments, the provided methods associated with formulation of cells include processing transduced cells, such as cells transduced and/or expanded using the processing steps described above, in a closed system.

In certain embodiments, one or more compositions of enriched T cells, such as engineered and cultivated T cells, e.g., output T cells, therapeutic cell composition, are formulated. In particular embodiments, one or more compositions of enriched T cells, such as engineered and cultivated T cells, e.g., output T cells, therapeutic cell composition, are formulated after the one or more compositions have been engineered and/or cultivated. In particular embodiments, the one or more compositions are input compositions. In some embodiments, the one or more input compositions have been previously cryofrozen and stored, and are thawed prior to the incubation.

In certain embodiments, the one or more therapeutic compositions of enriched T cells, such as engineered and cultivated T cells, e.g., output T cells, are or include two separate compositions, e.g., separate engineered and/or cultivated compositions, of enriched T cells. In particular embodiments, two separate therapeutic compositions of enriched T cells, e.g., two separate compositions of enriched CD4+ T cells and CD8+ T cells selected, isolated, and/or enriched from the same biological sample, separately engineered and separately cultivated, are separately formulated. In certain embodiments, the two separate therapeutic cell compositions include a composition of enriched CD4+ T cells, such as a composition of engineered and/or cultivated CD4+ T cells. In particular embodiments, the two separate therapeutic cell compositions include a composition of enriched CD8+ T cells, such as a composition of engineered and/or cultivated CD8+ T cells. In some embodiments, two separate therapeutic compositions of enriched CD4+ T cells and enriched CD8+ T cells, such as separate compositions of engineered and cultivated CD4+ T cells and engineered and cultivated CD8+ T cells, are separately formulated. In some embodiments, a single therapeutic composition of enriched T cells is formulated. In certain embodiments, the single therapeutic composition is a composition of enriched CD4+ T cells, such as a composition of engineered and/or cultivated CD4+ T cells. In some embodiments, the single therapeutic composition is a composition of enriched CD4+ and CD8+ T cells that have been combined from separate compositions prior to the formulation.

In some embodiments, separate therapeutic compositions of enriched CD4+ and CD8+ T cells, such as separate compositions of engineered and cultivated CD4+ and CD8+ T cells, are combined into a single therapeutic composition and are formulated. In certain embodiments, separate formulated therapeutic compositions of enriched CD4+ and enriched CD8+ T cells are combined into a single therapeutic composition after the formulation has been performed and/or completed. In particular embodiments, separate therapeutic compositions of enriched CD4+ and CD8+ T cells, such as separate compositions of engineered and cultivated CD4+ and CD8+ T cells, are separately formulated as separate compositions.

In some embodiments, the therapeutic composition of enriched CD4+ T cells, such as an engineered and cultivated CD4+ T cells, e.g., output CD4+ T cells, that is formulated, includes at least 60%, at least 65%, at least 70%, at least 75%, at least 80%, at least 85%, at least 90%, at least 95%, at least 98%, at least 99%, at least 99.5%, at least 99.9%, or at or at about 100% CD4+ T cells. In some embodiments, the composition includes at least 30%, at least 40%, at least 50%, at least 60%, at least 70%, at least 80%, at least 90%, at least 95%, at least 98%, at least 99%, at least 99.5%, at least 99.9%, or at or at about 100% CD4+ T cells that express a recombinant receptor and/or have been transduced or transfected with the recombinant polynucleotide. In certain embodiments, the therapeutic composition of enriched CD4+ T cells, such as an engineered and cultivated CD4+ T cells, e.g., output CD4+ T cells, that is formulated includes less than 40%, less than 35%, less than 30%, less than 25%, less than 20%, less than 15%, less than 10%, less than 5%, less than 1%, less than 0.1%, or less than 0.01% CD8+ T cells, and/or contains no CD8+ T cells, and/or is free or substantially free of CD8+ T cells.

In some embodiments, the therapeutic composition of enriched CD8+ T cells, such as an engineered and cultivated CD8+ T cells, e.g., output CD8+ T cells, that is formulated, includes at least 60%, at least 65%, at least 70%, at least 75%, at least 80%, at least 85%, at least 90%, at least 95%, at least 98%, at least 99%, at least 99.5%, at least 99.9%, or at or at about 100% CD8+ T cells. In certain embodiments, the therapeutic composition includes at least 30%, at least 40%, at least 50%, at least 60%, at least 70%, at least 80%, at least 90%, at least 95%, at least 98%, at least 99%, at least 99.5%, at least 99.9%, or at or at about 100% CD8+ T cells that express the recombinant receptor and/or have been transduced or transfected with the recombinant polynucleotide. In certain embodiments, the therapeutic composition of enriched CD8+ T cells, such as an engineered and cultivated CD8+ T cells, e.g., output CD8+ T cells, that is incubated under stimulating conditions includes less than 40%, less than 35%, less than 30%, less than 25%, less than 20%, less than 15%, less than 10%, less than 5%, less than 1%, less than 0.1%, or less than 0.01% CD4+ T cells, and/or contains no CD4+ T cells, and/or is free or substantially free of CD4+ T cells.

In some embodiments, features of the one or more therapeutic compositions are assessed, for example as described in Sections I-A and I-A-3, prior to formulation. In some embodiments, the features are cell phenotypes and recombinant receptor-dependent activity. In some embodiments, the cell phenotypes and recombinant receptor-dependent activity, are quantified to provide a number, percentage, proportion, and/or ratio of cells having an attribute in the therapeutic cell composition. In some embodiments, the features are used as input to a machine learning process provided herein.

In certain embodiments, the formulated cells are output cells. In some embodiments, a formulated therapeutic composition of enriched T cells, such as a formulated composition of engineered and cultivated T cells, is an output composition of enriched T cells. In particular embodiments, the formulated CD4+ T cells and/or formulated CD8+ T cells are the output CD4+ and/or CD8+ T cells. In particular embodiments, a formulated composition of enriched CD4+ T cells is an output composition of enriched CD4+ T cells. In some embodiments, a formulated composition of enriched CD8+ T cells is an output composition of enriched CD8+ T cells.

In some embodiments, cells can be formulated into a container, such as a bag or vial. In some embodiments, the cells are formulated between 0 days and 10 days, between 0 and 5 days, between 2 days and 7 days, between 0.5 days, and 4 days, or between 1 day and 3 days after the cells after the threshold cell count, density, and/or expansion has been achieved during the cultivation. In certain embodiments, the cells are formulated at or at or about or within 12 hours, 18 hours, 24 hours, 1 day, 2 days, or 3 days after the threshold cell count, density, and/or expansion has been achieved during the cultivation. In some embodiments, the cells are formulated within or within about 1 day after the threshold cell count, density, and/or expansion has been achieved during the cultivation.

Particular embodiments contemplate that cells are in a more activated state at early stages during the cultivation than at later stages during the cultivation. Further, in some embodiments, it may be desirable to formulate cells that are in a less activated state than the peak activation that occurs or may occur during the cultivation. In certain embodiments, the cells are cultivated for a minimum duration or amount of time, for example, so that cells are harvested in a less activated state than if they were formulated at an earlier time point during the cultivation, regardless of when the threshold is achieved. In some embodiments, the cells are cultivated between 1 day and 3 days after the threshold cell count, density, and/or expansion has been achieved during the cultivation. In certain embodiments, the cells achieve the threshold cell count, density, and/or expansion and remain cultivated for a minimum time or duration prior to the formulation. In some embodiments, cells that have achieved the threshold are not formulated until they have been cultivated for a minimum duration and/or amount of time, such as a minimum time or duration of between 1 day and 14 days, 2 days and 7 days, or 3 days and 6 days, or a minimum time or duration of the cultivation of or of about 2 days, 3 days, 4 days, 5 days, 6 days, 7 days, or more than 7 days. In some embodiments, the minimum time or duration of the cultivation is between 3 days and 6 days.

In some embodiments, the cells are formulated in a pharmaceutically acceptable buffer, which may, in some aspects, include a pharmaceutically acceptable carrier or excipient. In some embodiments, the processing includes exchange of a medium into a medium or formulation buffer that is pharmaceutically acceptable or desired for administration to a subject. In some embodiments, the processing steps can involve washing the transduced and/or expanded cells to replace the cells in a pharmaceutically acceptable buffer that can include one or more optional pharmaceutically acceptable carriers or excipients. Exemplary of such pharmaceutical forms, including pharmaceutically acceptable carriers or excipients, can be any described below in conjunction with forms acceptable for administering the cells and compositions to a subject. The pharmaceutical composition in some embodiments contains the cells in amounts effective to treat or prevent the disease or condition, such as a therapeutically effective or prophylactically effective amount.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular cell and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.00010% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being treated with the cells, preferably those with activities complementary to the cells, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine.

Compositions in some embodiments are provided as sterile liquid preparations, e.g., isotonic aqueous solutions, suspensions, emulsions, dispersions, or viscous compositions, which may in some aspects be buffered to a selected pH. Liquid compositions can comprise carriers, which can be a solvent or dispersing medium containing, for example, water, saline, phosphate buffered saline, polyol (for example, glycerol, propylene glycol, liquid polyethylene glycol) and suitable mixtures thereof. Sterile injectable solutions can be prepared by incorporating the cells in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

In some embodiments, the formulation buffer contains a cryopreservative. In some embodiments, the cell are formulated with a cryopreservative solution that contains 1.0% to 30% DMSO solution, such as a 5% to 20% DMSO solution or a 5% to 10% DMSO solution. In some embodiments, the cryopreservation solution is or contains, for example, PBS containing 20% DMSO and 8% human serum albumin (HSA), or other suitable cell freezing media. In some embodiments, the cryopreservative solution is or contains, for example, at least or about 7.5% DMSO. In some embodiments, the processing steps can involve washing the transduced and/or expanded cells to replace the cells in a cryopreservative solution. In some embodiments, the cells are frozen, e.g., cryofrozen or cryopreserved, in media and/or solution with a final concentration of or of about 12.5%, 12.0%, 11.5%, 11.0%, 10.5%, 10.0%, 9.5%, 9.0%, 8.5%, 8.0%, 7.5%, 7.0%, 6.5%, 6.0%, 5.5%, or 5.0% DMSO, or between 1% and 15%, between 6% and 12%, between 5% and 10%, or between 6% and 8% DMSO. In particular embodiments, the cells are frozen, e.g., cryofrozen or cryopreserved, in media and/or solution with a final concentration of or of about 5.0%, 4.5%, 4.0%, 3.5%, 3.0%, 2.5%, 2.0%, 1.5%, 1.25%, 1.0%, 0.75%, 0.5%, or 0.25% HSA, or between 0.1% and −5%, between 0.25% and 4%, between 0.5% and 2%, or between 1% and 2% HSA.

In particular embodiments, the therapeutic composition of enriched T cells, e.g., T cells that have been stimulated, engineered, and/or cultivated, are formulated, cryofrozen, and then stored for an amount of time. In certain embodiments, the formulated, cryofrozen cells are stored until the cells are released for infusion. In particular embodiments, the formulated cryofrozen cells are stored for between 1 day and 6 months, between 1 month and 3 months, between 1 day and 14 days, between 1 day and 7 days, between 3 days and 6 days, between 6 months and 12 months, or longer than 12 months. In some embodiments, the cells are cryofrozen and stored for, for about, or for less than 1 days, 2 days, 3 days, 4 days, 5 days, 6 days, or 7 days. In certain embodiments, the cells are thawed and administered to a subject after the storage. In certain embodiments, the cells are stored for or for about 5 days.

In some embodiments, the formulation is carried out using one or more processing step including washing, diluting or concentrating the cells, such as the cultured or expanded cells. In some embodiments, the processing can include dilution or concentration of the cells to a desired concentration or number, such as unit dose form compositions including the number of cells for administration in a given dose or fraction thereof. In some embodiments, the processing steps can include a volume-reduction to thereby increase the concentration of cells as desired. In some embodiments, the processing steps can include a volume-addition to thereby decrease the concentration of cells as desired. In some embodiments, the processing includes adding a volume of a formulation buffer to transduced and/or expanded cells. In some embodiments, the volume of formulation buffer is from 10 mL to 1000 mL or from about 10 mL to about 1000 mL, such as at least or about at least or about 50 mL, 100 mL, 200 mL, 300 mL, 400 mL, 500 mL, 600 mL, 700 mL, 800 mL, 900 mL or 1000 mL.

In some embodiments, such processing steps for formulating a cell composition is carried out in a closed system. Exemplary of such processing steps can be performed using a centrifugal chamber in conjunction with one or more systems or kits associated with a cell processing system, such as a centrifugal chamber produced and sold by Biosafe SA, including those for use with the Sepax® or Sepax 2® cell processing systems. An exemplary system and process is described in International Publication Number WO2016/073602. In some embodiments, the method includes effecting expression from the internal cavity of the centrifugal chamber a formulated composition, which is the resulting composition of cells formulated in a formulation buffer, such as pharmaceutically acceptable buffer, in any of the above embodiments as described. In some embodiments, the expression of the formulated composition is to a container, such as the vials of the biomedical material vessels described herein, that is operably linked as part of a closed system with the centrifugal chamber. In some embodiments, the biomedical material vessels are configured for integration and/or operable connection and/or is integrated or operably connected, to a closed system or device that carries out one or more processing steps. In some embodiments, the biomedical material vessel is connected to a system at an output line or output position. In some cases, the closed system is connected to the vial of the biomedical material vessel at the inlet tube. Exemplary close systems for use with the biomedical material vessels described herein include the Sepax® and Sepax® 2 system.

In some embodiments, the closed system, such as associated with a centrifugal chamber or cell processing system, includes a multi-port output kit containing a multi-way tubing manifold associated at each end of a tubing line with a port to which one or a plurality of containers can be connected for expression of the formulated composition. In some aspects, a desired number or plurality of vials, can be sterilely connected to one or more, generally two or more, such as at least 3, 4, 5, 6, 7, 8 or more of the ports of the multi-port output. For example, in some embodiments, one or more containers, e.g., biomedical material vessels, can be attached to the ports, or to fewer than all of the ports. Thus, in some embodiments, the system can effect expression of the output composition into a plurality of vials of the biomedical material vessels.

In some aspects, cells can be expressed to the one or more of the plurality of output containers, e.g., vials of the biomedical material vessels, in an amount for dosage administration, such as for a single unit dosage administration or multiple dosage administration. For example, in some embodiments, the vials of the biomedical material vessels, may each contain the number of cells for administration in a given dose or fraction thereof. Thus, each vial, in some aspects, may contain a single unit dose for administration or may contain a fraction of a desired dose such that more than one of the plurality of vials, such as two of the vials, or three of the vials, together constitutes a dose for administration.

Thus, the vials described herein, generally contain the cells to be administered, e.g., one or more unit doses thereof. The unit dose may be an amount or number of the cells to be administered to the subject or twice the number (or more) of the cells to be administered. It may be the lowest dose or lowest possible dose of the cells that would be administered to the subject.

In some embodiments, each of the containers, e.g., bags of vials individually comprises a unit dose of the cells. Thus in some embodiments, each of the containers comprises the same or approximately or substantially the same number of cells. In some embodiments, each unit dose contains at least or about at least 1×10⁶, 2×10⁶, 5×10⁶, 1×10⁷, 5×10⁷, or 1×10⁸ engineered cells, total cells, T cells, or PBMCs. In some embodiments, the volume of the formulated cell composition in each container, e.g., bag or vial, is 10 mL to 100 mL, such as at least or about at least or about 20 mL, 30 mL, 40 mL, 50 mL, 60 mL, 70 mL, 80 mL, 90 mL or 100 mL. In some embodiments, the cells in the container, e.g., bag or vials, can be cryopreserved. In some embodiments, the container, e.g., vials, can be stored in liquid nitrogen until further use.

In some embodiments, such cells produced by the method, or a composition comprising such cells, are administered to a subject for treating a disease or condition.

### III. Recombinant Receptors for Genetic Engineering

In some embodiments, the cells, e.g., CD4+ cells, CD8+ cells, of the T cell therapy, also referred to herein as a therapeutic cell composition, are engineered cells encoding a recombinant protein. In some embodiments, the engineered cells of the therapeutic cell composition contain or express, a recombinant protein, such as a recombinant receptor, e.g., a chimeric antigen receptor (CAR), or a T cell receptor (TCR). In certain embodiments, the methods for manufacturing or engineering described produce and/or are capable of producing cells, or populations or compositions containing and/or enriched for cells, that are engineered to express or contain a recombinant protein such as a recombinant receptor.

In some aspects, the encoded recombinant receptor is a chimeric antigen receptor (CAR) or a recombinant T cell receptor (TCR). Among the recombinant receptors are chimeric receptors, antigen receptors and receptors containing one or more component of chimeric receptors or antigen receptors. The recombinant receptors may include those containing ligand-binding domains or binding fragments thereof and intracellular signaling domains or regions. In some embodiments, the recombinant receptors encoded by the engineered cells include functional non-TCR antigen receptors, chimeric antigen receptors (CARs), chimeric autoantibody receptor (CAAR), recombinant T cell receptors (TCRs) and regions, domains or components of any of the foregoing, including one or more polypeptide chains of a multi-chain recombinant receptor. The recombinant receptor, such as a CAR, generally includes the extracellular antigen (or ligand) binding domain linked to one or more intracellular signaling components, in some aspects via linkers and/or transmembrane domain(s). In some embodiments, exemplary recombinant receptors expressed from the engineered cell include multi-chain receptors that contain two or more receptor polypeptides, which, in some cases, contain different components, domains or regions. In some aspects, the recombinant receptor contains two or more polypeptides that together comprise a functional recombinant receptor. In some aspects, the multi-chain receptor is a dual-chain receptor, comprising two polypeptides that together comprise a functional recombinant receptor. In some embodiments, the recombinant receptor is a TCR comprising two different receptor polypeptides, for example, a TCR alpha (TCRα) and a TCR beta (TCRβ) chain; or a TCR gamma (TCRγ) and a TCR delta (TCRδ) chain. In some embodiments, the recombinant receptor is a multi-chain receptor in which one or more of the polypeptides regulates, modifies or controls the expression, activity or function of another receptor polypeptide. In some aspects, multi-chain receptors allows spatial or temporal regulation or control of specificity, activity, antigen (or ligand) binding, function and/or expression of the receptor.

### A. Chimeric Antigen Receptors (CARs)

In some embodiments, genetically engineered receptors that redirect cytotoxicity of immune effector cells toward B cells are provided. These genetically engineered receptors referred to herein as chimeric antigen receptors (CARs). CARs are molecules that combine antibody-based specificity for a desired antigen (e.g., BCMA) with a T cell receptor-activating intracellular domain to generate a chimeric protein that exhibits a specific anti-BCMA cellular immune activity. As used herein, the term, “chimeric,” describes being composed of parts of different proteins or DNAs from different origins.

In some embodiments of the provided methods and uses, the engineered cells, such as T cells, express a chimeric receptor, such as a chimeric antigen receptor (CAR), that contains one or more domains that combine a ligand-binding domain (e.g. antibody or antibody fragment) that provides specificity for a desired antigen (e.g., tumor antigen) with intracellular signaling domains. In some embodiments, the intracellular signaling domain is an activating intracellular domain portion, such as a T cell activating domain, providing a primary activation signal. In some embodiments, the intracellular signaling domain contains or additionally contains a costimulatory signaling domain to facilitate effector functions. Upon specific binding to the molecule, e.g., antigen, the receptor generally delivers an immunostimulatory signal, such as an ITAM-transduced signal, into the cell, thereby promoting an immune response targeted to the disease or condition. In some embodiments, chimeric receptors when genetically engineered into immune cells can modulate T cell activity, and, in some cases, can modulate T cell differentiation or homeostasis, thereby resulting in genetically engineered cells with improved longevity, survival and/or persistence in vivo, such as for use in adoptive cell therapy methods.

The terms “complementarity determining region,” and “CDR,” synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4).

The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme); Al-Lazikani et al., (1997) JMB 273, 927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309(3):657-70, (“Aho” numbering scheme); and Martin et al., “Modeling antibody hypervariable loops: a combined algorithm,” PNAS, 1989, 86(23):9268-9272, (“AbM” numbering scheme).

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme. The AbM scheme is a compromise between Kabat and Chothia definitions based on that used by Oxford Molecular's AbM antibody modeling software.

Table 1, below, lists exemplary position boundaries of CDR-L1, CDR-L2, CDR-L3 and CDR-H1, CDR-H2, CDR-H3 as identified by Kabat, Chothia, AbM, and Contact schemes, respectively. For CDR-H1, residue numbering is listed using both the Kabat and Chothia numbering schemes. FRs are located between CDRs, for example, with FR-L1 located before CDR-L1, FR-L2 located between CDR-L1 and CDR-L2, FR-L3 located between CDR-L2 and CDR-L3 and so forth. It is noted that because the shown Kabat numbering scheme places insertions at H35A and H35B, the end of the Chothia CDR-H1 loop when numbered using the shown Kabat numbering convention varies between H32 and H34, depending on the length of the loop.

TABLE 1
Boundaries of CDRs according to various numbering schemes

| CDR | Kabat | Chothia | AbM | Contact |
|---|---|---|---|---|
| CDR-L1 | L24--L34 | L24--L34 | L24--L34 | L30--L36 |
| CDR-L2 | L50--L56 | L50--L56 | L50--L56 | L46--L55 |
| CDR-L3 | L89--L97 | L89--L97 | L89--L97 | L89--L96 |
| CDR-H1 | H31--H35B | H26--H32.34 | H26--H35B | H30--H35B |
| (Kabat | | | | |
| Numbering¹) | | | | |
| CDR-H1 | H31--H35 | H26--H32 | H26--H35 | H30--H35 |
| (Chothia | | | | |
| Numbering²) | | | | |
| CDR-H2 | H50--H65 | H52--H56 | H50--H58 | H47--H58 |
| CDR-H3 | H95--H102 | H95--H102 | H95--H102 | H93--H101 |

¹Kabat et al. (1991), “Sequences of Proteins of Immunological Interest,” 5th Ed. Public Health Service, National Institutes of Health, Bethesda, MD
²Al-Lazikani et al., (1997) JMB 273, 927-948

Thus, unless otherwise specified, a “CDR” or “complementary determining region,” or individual specified CDRs (e.g., CDR-H1, CDR-H2, CDR-H3), of a given antibody or region thereof, such as a variable region thereof, should be understood to encompass a (or the specific) complementary determining region as defined by any of the aforementioned schemes, or other known schemes. For example, where it is stated that a particular CDR (e.g., a CDR-H3) contains the amino acid sequence of a corresponding CDR in a given V_{H} or V_{L} region amino acid sequence, it is understood that such a CDR has a sequence of the corresponding CDR (e.g., CDR-H3) within the variable region, as defined by any of the aforementioned schemes, or other known schemes. In some embodiments, specific CDR sequences are specified. Exemplary CDR sequences of provided antibodies are described using various numbering schemes, although it is understood that a provided antibody can include CDRs as described according to any of the other aforementioned numbering schemes or other numbering schemes known to a skilled artisan.

Likewise, unless otherwise specified, a FR or individual specified FR(s) (e.g., FR-H1, FR-H2, FR-H3, FR-H4), of a given antibody or region thereof, such as a variable region thereof, should be understood to encompass a (or the specific) framework region as defined by any of the known schemes. In some instances, the scheme for identification of a particular CDR, FR, or FRs or CDRs is specified, such as the CDR as defined by the Kabat, Chothia, AbM, IMGT or Contact method, or other known schemes. In other cases, the particular amino acid sequence of a CDR or FR is given.

Antibody fragments can be made by various techniques, including but not limited to proteolytic digestion of an intact antibody as well as production by recombinant host cells. In some embodiments, the antibodies are recombinantly produced fragments, such as fragments comprising arrangements that do not occur naturally, such as those with two or more antibody regions or chains joined by synthetic linkers, e.g., peptide linkers, and/or that are may not be produced by enzyme digestion of a naturally-occurring intact antibody. In some aspects, the antibody fragments are scFv.

CAR T cell therapies to which the embodiments described herein apply include any CAR T therapy, such as BCMA CAR T cell therapies, such as BCMA02, JCARH125, JNJ-68284528 (LCAR-B38M; cilta-cel; CARVICTY™) (Janssen/Legend), P-BCMA-101 (Poseida), PBCAR269A (Poseida), P-BCMA-Allo1 (Poseida), Allo-715 (Pfizer/Allogene), CT053 (Carsgen), Descartes-08 (Cartesian), PHE885 (Novartis), ARI-002 (Hospital Clinic Barcelona, IDIBAPS), CTX120 (CRISPR Therapeutics); CD19 CAR T therapies, e.g., Yescarta, Kymriah, Tecartus, lisocabtagene maraleucel (liso-cel), and CAR T therapies targeting any other cell surface marker.

The extracellular domain (also referred to as a binding domain or antigen-specific binding domain) of the polypeptide binds to an antigen of interest. In certain embodiments, the extracellular domain comprises a receptor, or a portion of a receptor, that binds to said antigen. The extracellular domain may be, e.g., a receptor, or a portion of a receptor, that binds to said antigen. In certain embodiments, the extracellular domain comprises, or is, an antibody or an antigen-binding portion thereof. In specific embodiments, the extracellular domain comprises, or is, a single-chain Fv domain. The single-chain Fv domain can comprise, for example, a V_{L} linked to V_{H} by a flexible linker, wherein said V_{L} and V_{H} are from an antibody that binds said antigen.

The antigen to which the extracellular domain of the polypeptide binds can be any antigen of interest, e.g., can be an antigen on a tumor cell. The tumor cell may be, e.g., a cell in a solid tumor, or a cell of a blood cancer. The antigen can be any antigen that is expressed on a cell of any tumor or cancer type, e.g., cells of a lymphoma, a leukemia, a lung cancer, a breast cancer, a prostate cancer, a liver cancer, a cholangiocarcinoma, a glioma, a colon adenocarcinoma, a myelodysplasia, an adrenocortical carcinoma, a thyroid carcinoma, a nasopharyngeal carcinoma, a melanoma, e.g., a malignant melanoma, a skin carcinoma, a colorectal carcinoma, a desmoid tumor, a desmoplastic small round cell tumor, an endocrine tumor, an Ewing sarcoma, a peripheral primitive neuroectodermal tumor, a solid germ cell tumor, a hepatoblastoma, a neuroblastoma, a non-rhabdomyosarcoma soft tissue sarcoma, an osteosarcoma, a retinoblastoma, a rhabdomyosarcoma, a Wilms tumor, a glioblastoma, a myxoma, a fibroma, a lipoma, or the like. In more specific embodiments, said lymphoma can be chronic lymphocytic leukemia (small lymphocytic lymphoma), B-cell prolymphocytic leukemia, lymphoplasmacytic lymphoma, Waldenstrom macroglobulinemia, splenic marginal zone lymphoma, plasma cell myeloma, plasmacytoma, extranodal marginal zone B cell lymphoma, MALT lymphoma, nodal marginal zone B cell lymphoma, follicular lymphoma, mantle cell lymphoma, diffuse large B cell lymphoma, mediastinal (thymic) large B cell lymphoma, intravascular large B cell lymphoma, primary effusion lymphoma, Burkitt's lymphoma, T lymphocyte prolymphocytic leukemia, acute myeloid leukemia (AML), acute lymphocytic leukemia (ALL), chronic myelogenous leukemia (CML), juvenile chronic myelogenous leukemia (JCML), juvenile myelomonocytic leukemia (JMML), T lymphocyte large granular lymphocytic leukemia, aggressive NK cell leukemia, adult T lymphocyte leukemia/lymphoma, extranodal NK/T lymphocyte lymphoma, nasal type, enteropathy-type T lymphocyte lymphoma, hepatosplenic T lymphocyte lymphoma, blastic NK cell lymphoma, mycosis fungoides, Sezary syndrome, primary cutaneous anaplastic large cell lymphoma, lymphomatoid papulosis, angioimmunoblastic T lymphocyte lymphoma, peripheral T lymphocyte lymphoma (unspecified), anaplastic large cell lymphoma, Hodgkin lymphoma, a non-Hodgkin lymphoma, or multiple myeloma.

In certain embodiments, the antigen is a tumor-associated antigen (TAA) or a tumor-specific antigen (TSA). In various specific embodiments, without limitation, the tumor-associated antigen or tumor-specific antigen is Her2, prostate stem cell antigen (PSCA), alpha-fetoprotein (AFP), carcinoembryonic antigen (CEA), cancer antigen-125 (CA-125), CA19-9, calretinin, MUC-1, epithelial membrane protein (EMA), epithelial tumor antigen (ETA), tyrosinase, melanoma-associated antigen (MAGE), CD19, CD20, CD34, CD45, CD99, CD 117, chromogranin, cytokeratin, desmin, glial fibrillary acidic protein (GFAP), gross cystic disease fluid protein (GCDFP-15), HMB-45 antigen, high molecular weight melanoma-associated antigen (HMW-MAA), protein melan-A (MART-1), myo-D1, muscle-specific actin (MSA), neurofilament, neuron-specific enolase (NSE), placental alkaline phosphatase, synaptophysis, thyroglobulin, thyroid transcription factor-1, the dimeric form of the pyruvate kinase isoenzyme type M2 (tumor M2-PK), an abnormal ras protein, or an abnormal p53 protein.

In certain embodiments, the TAA or TSA is a cancer/testis (CT) antigen, e.g., BAGE, CAGE, CTAGE, FATE, GAGE, HCA661, HOM-TES-85, MAGEA, MAGEB, MAGEC, NA88, NY-ESO-1, NY-SAR-35, OY-TES-1, SPANXB1, SPA17, SSX, SYCP1, or TPTE.

In certain other embodiments, the TAA or TSA is a carbohydrate or ganglioside, e.g., fuc-GM1, GM2 (oncofetal antigen-immunogenic-1; OFA-I-1); GD2 (OFA-I-2), GM3, GD3, and the like.

In certain other embodiments, the TAA or TSA is alpha-actinin-4, Bage-1, BCR-ABL, Bcr-Abl fusion protein, beta-catenin, CA 125, CA 15-3 (CA 27.29\BCAA), CA 195, CA 242, CA-50, CAM43, Casp-8, cdc27, cdk4, cdkn2a, CEA, coa-1, dek-can fusion protein, EBNA, EF2, Epstein Barr virus antigens, ETV6-AML1 fusion protein, HLA-A2, HLA-A11, hsp70-2, KIAAO205, Mart2, Mum-1, 2, and 3, neo-PAP, myosin class I, OS-9, pml-RARα fusion protein, PTPRK, K-ras, N-ras, triosephosphate isomerase, Gage 3,4,5,6,7, GnTV, Herv-K-mel, Lage-1, NA-88, NY-Eso-1/Lage-2, SP17, SSX-2, TRP2-Int2, gp100 (Pmel 17), tyrosinase, TRP-1, TRP-2, MAGE-1, MAGE-3, RAGE, GAGE-1, GAGE-2, p15(58), RAGE, SCP-1, Hom/Mel-40, PRAME, p53, H-Ras, HER-2/neu, E2A-PRL, H4-RET, IGH-IGK, MYL-RAR, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, 13-Catenin, Mum-1, p16, TAGE, PSMA, CT7, telomerase, 43-9F, 5T4, 791Tgp72, 13HCG, BCA225, BTAA, CD68KP1, CO-029, FGF-5, G250, Ga733 (EpCAM), HTgp-175, M344, MA-50, MG7-Ag, MOV18, NB70K, NY-CO-1, RCAS1, SDCCAG16, TA-90, TAAL6, TAG72, TLP, TPS, CD19, CD22, CD27, CD30, CD70, GD2 (ganglioside G2), EGFRvIII (epidermal growth factor variant III), sperm protein 17 (Sp17), mesothelin, PAP (prostatic acid phosphatase), prostein, TARP (T cell receptor gamma alternate reading frame protein), Trp-p8, STEAP1 (six-transmembrane epithelial antigen of the prostate 1), an abnormal ras protein, or an abnormal p53 protein. In another specific embodiment, said tumor-associated antigen or tumor-specific antigen is integrin αvβ3 (CD61), galactin, K-Ras (V-Ki-ras2 Kirsten rat sarcoma viral oncogene), or Ral-B.

In specific embodiments, the TAA or TSA is CD20, CD123, CLL-1, CD38, CS-1, CD138, ROR1, FAP, MUC1, PSCA, EGFRvIII, EPHA2, or GD2. In further specific embodiments, the TAA or TSA is CD123, CLL-1, CD38, or CS-1. In a specific embodiment, the extracellular domain of the CAR binds CS-1. In a further specific embodiment, the extracellular domain comprises a single-chain version of elotuzumab and/or an antigen-binding fragment of elotuzumab. In a specific embodiment, the extracellular domain of the CAR binds CD20. In a more specific embodiment, the extracellular domain of the CAR is an scFv or antigen-binding fragment thereof binds to CD20.

Other tumor-associated and tumor-specific antigens are known to those in the art.

Antibodies and scFvs, that bind to TSAs and TAAs are known in the art, as are nucleotide sequences that encode them.

In certain specific embodiments, the antigen is an antigen not considered to be a TSA or a TAA, but which is nevertheless associated with tumor cells, or damage caused by a tumor. In specific embodiments, the antigen is a tumor microenvironment-associated antigen (TMAA). In certain embodiments, for example, the TMAA is, e.g., a growth factor, cytokine or interleukin, e.g., a growth factor, cytokine, or interleukin associated with angiogenesis or vasculogenesis. Such growth factors, cytokines, or interleukins can include, e.g., vascular endothelial growth factor (VEGF), basic fibroblast growth factor (bFGF), platelet-derived growth factor (PDGF), hepatocyte growth factor (HGF), insulin-like growth factor (IGF), or interleukin-8 (IL-8). Tumors can also create a hypoxic environment local to the tumor. As such, in other specific embodiments, the TMAA is a hypoxia-associated factor, e.g., HIF-1α, HIF-1β, HIF-2α, HIF-2β, HIF-3α, or HIF-3β. Tumors can also cause localized damage to normal tissue, causing the release of molecules known as damage associated molecular pattern molecules (DAMPs; also known as alarmins). In certain other specific embodiments, therefore, the TMAA is a DAMP, e.g., a heat shock protein, chromatin-associated protein high mobility group box 1 (HMGB1), S100A8 (MRP8, calgranulin A), S100A9 (MRP14, calgranulin B), serum amyloid A (SAA), or can be a deoxyribonucleic acid, adenosine triphosphate, uric acid, or heparin sulfate. In specific embodiments, the TMAA is VEGF-A, EGF, PDGF, IGF, or bFGF.

In certain embodiments, the extracellular domain is joined to said transmembrane domain by a linker, spacer or hinge polypeptide sequence, e.g., a sequence from CD28.

In certain embodiments, CARs contemplated herein, comprise an extracellular domain that binds to BCMA, a transmembrane domain, and an intracellular signaling domain. Engagement of the anti-BCMA antigen binding domain of the CAR with BCMA on the surface of a target cell results in clustering of the CAR and delivers an activation stimulus to the CAR-containing cell. The main characteristic of CARs are their ability to redirect immune effector cell specificity, thereby triggering proliferation, cytokine production, phagocytosis or production of molecules that can mediate cell death of the target antigen expressing cell in a major histocompatibility (MHC) independent manner, exploiting the cell specific targeting abilities of monoclonal antibodies, soluble ligands or cell specific co-receptors.

In various embodiments, a CAR comprises an extracellular binding domain that comprises a murine anti-BCMA (e.g., human BCMA)-specific binding domain; a transmembrane domain; one or more intracellular co-stimulatory signaling domains; and a primary signaling domain.

In particular embodiments, a CAR comprises an extracellular binding domain that comprises a murine anti-BCMA (e.g., human BCMA) antibody or antigen binding fragment thereof; one or more hinge domains or spacer domains; a transmembrane domain including; one or more intracellular co-stimulatory signaling domains; and a primary signaling domain.

The present disclosure contemplates, in part, CAR polypeptides and fragments thereof, cells and compositions comprising the same, and vectors that express polypeptides. In certain embodiments, a polypeptide contemplated herein comprises a CAR polypeptide. In particular embodiments, the CAR polypeptide sequence is set forth in SEQ ID NO:9 is provided. In particular embodiments, a CAR polypeptide sequence set forth in SEQ ID NO:37 is provided.

“Polypeptide,” “polypeptide fragment,” “peptide” and “protein” are used interchangeably, unless specified to the contrary, and according to conventional meaning, i.e., as a sequence of amino acids. Polypeptides are not limited to a specific length, e.g., they may comprise a full length protein sequence or a fragment of a full length protein, and may include post-translational modifications of the polypeptide, for example, glycosylations, acetylations, phosphorylations and the like, as well as other modifications known in the art, both naturally occurring and non-naturally occurring. In various embodiments, the CAR polypeptides contemplated herein comprise a signal (or leader) sequence at the N-terminal end of the protein, which co-translationally or post-translationally directs transfer of the protein. Illustrative examples of suitable signal sequences useful in CARs disclosed herein include, but are not limited to, the IgG1 heavy chain signal sequence and the CD8α signal sequence. Polypeptides can be prepared using any of a variety of well-known recombinant and/or synthetic techniques. Polypeptides contemplated herein specifically encompass the CARs of the present disclosure, or sequences that have deletions from, additions to, and/or substitutions of one or more amino acid of a CAR as disclosed herein.

An “isolated peptide” or an “isolated polypeptide” and the like, as used herein, refer to in vitro isolation and/or purification of a peptide or polypeptide molecule from a cellular environment, and from association with other components of the cell, i.e., it is not significantly associated with in vivo substances. Similarly, an “isolated cell” refers to a cell that has been obtained from an in vivo tissue or organ and is substantially free of extracellular matrix.

In some embodiments, the CAR has a polypeptide sequence that is a variant of any of the CARs as described herein. Polypeptides include “polypeptide variants.” Polypeptide variants may differ from a naturally occurring polypeptide in one or more substitutions, deletions, additions and/or insertions. Such variants may be naturally occurring or may be synthetically generated, for example, by modifying one or more of the above polypeptide sequences. For example, in particular embodiments, it may be desirable to improve the binding affinity and/or other biological properties of the CARs by introducing one or more substitutions, deletions, additions and/or insertions into a binding domain, hinge, TM domain, co-stimulatory signaling domain or primary signaling domain of a CAR polypeptide. In certain embodiments, such polypeptides include polypeptides having at least about 65%, 70%, 75%, 85%, 90%, 95%, 98%, or 99% amino acid identity thereto.

Polypeptides include “polypeptide fragments.” Polypeptide fragments refer to a polypeptide, which can be monomeric or multimeric, that has an amino-terminal deletion, a carboxyl-terminal deletion, and/or an internal deletion or substitution of a naturally-occurring or recombinantly-produced polypeptide. In certain embodiments, a polypeptide fragment can comprise an amino acid chain at least 5 to about 500 amino acids long. It will be appreciated that in certain embodiments, fragments are at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 150, 200, 250, 300, 350, 400, or 450 amino acids long. Particularly useful polypeptide fragments include functional domains, including antigen-binding domains or fragments of antibodies. In the case of a murine anti-BCMA (e.g., human BCMA) antibody, useful fragments include, but are not limited to: a CDR region, a CDR3 region of the heavy or light chain; a variable region of a heavy or light chain; a portion of an antibody chain or variable region including two CDRs; and the like.

The polypeptide may also be fused in-frame or conjugated to a linker or other sequence for ease of synthesis, purification or identification of the polypeptide (e.g., poly-His), or to enhance binding of the polypeptide to a solid support.

As noted above, polypeptides of the present disclosure may be altered in various ways including amino acid substitutions, deletions, truncations, and insertions. Methods for such manipulations are generally known in the art. For example, amino acid sequence variants of a reference polypeptide can be prepared by mutations in the DNA. Methods for mutagenesis and nucleotide sequence alterations are well known in the art. See, for example, Kunkel (1985, Proc. Natl. Acad. Sci. USA. 82: 488-492), Kunkel et al., (1987, Methods in Enzymol, 154: 367-382), U.S. Pat. No. 4,873,192, Watson, J. D. et al., (Molecular Biology of the Gene, Fourth Edition, Benjamin/Cummings, Menlo Park, Calif, 1987) and the references cited therein. Guidance as to appropriate amino acid substitutions that do not affect biological activity of the protein of interest may be found in the model of Dayhoff et al., (1978) Atlas of Protein Sequence and Structure (Natl. Biomed. Res. Found., Washington, D.C.).

In certain embodiments, a variant will contain conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. Modifications may be made in the structure of the polynucleotides and polypeptides of the present disclosure and still obtain a functional molecule that encodes a variant or derivative polypeptide with desirable characteristics. When it is desired to alter the amino acid sequence of a polypeptide to create an equivalent, or even an improved, variant polypeptide, one skilled in the art, for example, can change one or more of the codons of the encoding DNA sequence, e.g., according to Table 2.

TABLE 2
Amino Acid Codons

| | One | Three | | | | | | |
|---|---|---|---|---|---|---|---|---|
| | letter | letter | | | | | | |
| Amino Acids | code | code | Codons | | | | | |
| Alanine | A | Ala | GCA | GCC | GCG | GCU | | |
| Cysteine | C | Cys | UGC | UGU | | | | |
| Aspartic acid | D | Asp | GAC | GAU | | | | |
| Glutamic acid | E | Glu | GAA | GAG | | | | |
| Phenylalanine | F | Phe | UUC | UUU | | | | |
| Glycine | G | Gly | GGA | GGC | GGG | GGU | | |
| Histidine | H | His | CAC | CAU | | | | |
| Isoleucine | I | Ile | AUA | AUC | AUU | | | |
| Lysine | K | Lys | AAA | AAG | | | | |
| Leucine | L | Leu | UUA | UUG | CUA | CUC | CUG | CUU |
| Methionine | M | Met | AUG | | | | | |
| Asparagine | N | Asn | AAC | AAU | | | | |
| Proline | P | Pro | CCA | CCC | CCG | CCU | | |
| Glutamine | Q | Gln | CAA | CAG | | | | |
| Arginine | R | Arg | AGA | AGG | CGA | CGC | CGG | CGU |
| Serine | S | Ser | AGC | AGU | UCA | UCC | UCG | UCU |

| | | | | | | |
|---|---|---|---|---|---|---|
| Threonine | T | Thr | ACA | ACC | ACG | ACU |
| Valine | V | Val | GUA | GUC | GUG | GUU |

| Tryptophan | W | Trp | UGG | |
|---|---|---|---|---|
| Tyrosine | Y | Tyr | UAC | UAU |

Guidance in determining which amino acid residues can be substituted, inserted, or deleted without abolishing biological activity can be found using computer programs well known in the art, such as DNASTAR™ software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cysteine, serine, threonine, tyrosine) amino acids. Phenylalanine, tryptophan, and tyrosine are sometimes classified jointly as aromatic amino acids. In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224). Exemplary conservative substitutions are described in U.S. Provisional Patent Application No. 61/241,647, the disclosure of which is herein incorporated by reference.

In making such changes, the hydropathic index of amino acids may be considered. The importance of the hydropathic amino acid index in conferring interactive biologic function on a protein is generally understood in the art (Kyte and Doolittle, 1982, incorporated herein by reference). Each amino acid has been assigned a hydropathic index on the basis of its hydrophobicity and charge characteristics (Kyte and Doolittle, 1982). These values are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions may be based on the relative similarity of the amino acid side-chain substituents, for example, their hydrophobicity, hydrophilicity, charge, size, and the like.

Polypeptide variants further include glycosylated forms, aggregative conjugates with other molecules, and covalent conjugates with unrelated chemical moieties (e.g., pegylated molecules). Covalent variants can be prepared by linking functionalities to groups which are found in the amino acid chain or at the N- or C-terminal residue, as is known in the art. Variants also include allelic variants, species variants, and muteins. Truncations or deletions of regions which do not affect functional activity of the proteins are also variants.

In one embodiment, where expression of two or more polypeptides is desired, the polynucleotide sequences encoding them can be separated by and IRES sequence as discussed elsewhere herein. In certain embodiments, two or more polypeptides can be expressed as a fusion protein that comprises one or more self-cleaving polypeptide sequences.

Polypeptides disclosed herein include fusion polypeptides. In certain embodiments, fusion polypeptides and polynucleotides encoding fusion polypeptides are provided, e.g., CARs. Fusion polypeptides and fusion proteins refer to a polypeptide having at least two, three, four, five, six, seven, eight, nine, or ten or more polypeptide segments. Fusion polypeptides are typically linked C-terminus to N-terminus, although they can also be linked C-terminus to C-terminus, N-terminus to N-terminus, or N-terminus to C-terminus. The polypeptides of the fusion protein can be in any order or a specified order. Fusion polypeptides or fusion proteins can also include conservatively modified variants, polymorphic variants, alleles, mutants, subsequences, and interspecies homologs, so long as the desired transcriptional activity of the fusion polypeptide is preserved. Fusion polypeptides may be produced by chemical synthetic methods or by chemical linkage between the two moieties or may generally be prepared using other standard techniques. Ligated DNA sequences comprising the fusion polypeptide are operably linked to suitable transcriptional or translational control elements as discussed elsewhere herein.

In one embodiment, a fusion partner comprises a sequence that assists in expressing the protein (an expression enhancer) at higher yields than the native recombinant protein. Other fusion partners may be selected so as to increase the solubility of the protein or to enable the protein to be targeted to desired intracellular compartments or to facilitate transport of the fusion protein through the cell membrane.

Fusion polypeptides may further comprise a polypeptide cleavage signal between each of the polypeptide domains described herein. In addition, a polypeptide site can be put into any linker peptide sequence. Exemplary polypeptide cleavage signals include polypeptide cleavage recognition sites such as protease cleavage sites, nuclease cleavage sites (e.g., rare restriction enzyme recognition sites, self-cleaving ribozyme recognition sites), and self-cleaving viral oligopeptides (see deFelipe and Ryan, 2004. Traffic 5(8); 616-26).

Suitable protease cleavages sites and self-cleaving peptides are known to the skilled person (see, e.g., in Ryan et al., 1997. J Gener. Virol. 78, 699-722; Scymczak et al. (2004) Nature Biotech. 5, 589-594). Exemplary protease cleavage sites include, but are not limited to, the cleavage sites of potyvirus NIa proteases (e.g., tobacco etch virus protease), potyvirus HC proteases, potyvirus P1 (P35) proteases, byovirus NIa proteases, byovirus RNA-2-encoded proteases, aphthovirus L proteases, enterovirus 2A proteases, rhinovirus 2A proteases, picorna 3C proteases, comovirus 24K proteases, nepovirus 24K proteases, RTSV (rice tungro spherical virus) 3C-like protease, PYVF (parsnip yellow fleck virus) 3C-like protease, heparin, thrombin, factor Xa and enterokinase. Due to its high cleavage stringency, TEV (tobacco etch virus) protease cleavage sites are preferred in one embodiment, e.g., EXXYXQ (G/S) (SEQ ID NO:23), for example, ENLYFQG (SEQ ID NO:24) and ENLYFQS (SEQ ID NO:25), wherein X represents any amino acid (cleavage by TEV occurs between Q and G or Q and S).

In a particular embodiment, self-cleaving peptides include those polypeptide sequences obtained from potyvirus and cardiovirus 2A peptides, FMDV (foot-and-mouth disease virus), equine rhinitis A virus, Thosea asigna virus and porcine teschovirus.

In certain embodiments, the self-cleaving polypeptide site comprises a 2A or 2A-like site, sequence or domain (Donnelly et al., 2001. J Gen. Virol. 82:1027-1041).

TABLE 3
Exemplary 2A sites include the following sequences:

| | |
|---|---|
| SEQ ID NO: 26 | LLNFDLLKLAGDVESNPGP |
| SEQ ID NO: 27 | TLNFDLLKLAGDVESNPGP |
| SEQ ID NO: 28 | LLKLAGDVESNPGP |
| SEQ ID NO: 29 | NFDLLKLAGDVESNPGP |
| SEQ ID NO: 30 | QLLNFDLLKLAGDVESNPGP |
| SEQ ID NO: 31 | APVKQTLNFDLLKLAGDVESNPGP |
| SEQ ID NO: 32 | VTELLYRMKRAETYCPRPLLAIHPTEARHKQKIV |
| | APVKQT |
| SEQ ID NO: 33 | LNFDLLKLAGDVESNPGP |
| SEQ ID NO: 34 | LLAIHPTEARHKQKIVAPVKQTLNFDLLKLAGDV |
| | ESNPGP |
| SEQ ID NO: 35 | EARHKQKIVAPVKQTLNFDLLKLAGDVESNPGP |

### 1. Binding Domain

In particular embodiments, CARs contemplated herein comprise an extracellular binding domain that comprises a murine anti-BCMA antibody or antigen binding fragment thereof that specifically binds to a human BCMA polypeptide expressed on a B cell. As used herein, the terms, “binding domain,” “extracellular domain,” “extracellular binding domain,” “antigen-specific binding domain,” and “extracellular antigen specific binding domain,” are used interchangeably and provide a CAR with the ability to specifically bind to the target antigen of interest, e.g., BCMA. The binding domain may be derived either from a natural, synthetic, semi-synthetic, or recombinant source.

The terms “specific binding affinity” or “specifically binds” or “specifically bound” or “specific binding” or “specifically targets” as used herein, describe binding of an anti-BCMA antibody or antigen binding fragment thereof (or a CAR comprising the same) to BCMA at greater binding affinity than background binding. A binding domain (or a CAR comprising a binding domain or a fusion protein containing a binding domain) “specifically binds” to a BCMA if it binds to or associates with BCMA with an affinity or Kₐ (i.e., an equilibrium association constant of a particular binding interaction with units of 1/M) of, for example, greater than or equal to about 10⁵ M⁻¹. In certain embodiments, a binding domain (or a fusion protein thereof) binds to a target with a Kₐ greater than or equal to about 10⁶ M⁻¹, 10⁷ M⁻¹, 10⁸ M⁻¹, 10⁹ M⁻¹, 10¹⁰ M⁻¹, 10¹¹ M⁻¹, 10¹² M⁻¹, or 10¹³ M⁻¹. “High affinity” binding domains (or single chain fusion proteins thereof) refers to those binding domains with a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, at least 10¹³ M⁻¹, or greater. In some embodiments, a BCMA-Fc fusion polypeptide comprises the sequence set forth in SEQ ID NO:205.

Alternatively, affinity may be defined as an equilibrium dissociation constant (K_{d}) of a particular binding interaction with units of M (e.g., 10⁻⁵ M to 10⁻¹³ M, or less). Affinities of binding domain polypeptides and CAR proteins according to the present disclosure can be readily determined using conventional techniques, e.g., by competitive ELISA (enzyme-linked immunosorbent assay), or by binding association, or displacement assays using labeled ligands, or using a surface-plasmon resonance device such as the Biacore T100, which is available from Biacore, Inc., Piscataway, NJ, or optical biosensor technology such as the EPIC system or EnSpire that are available from Corning and Perkin Elmer respectively (see also, e.g., Scatchard et al. (1949) Ann. N.Y. Acad. Sci. 51:660; and U.S. Pat. Nos. 5,283,173; 5,468,614, or the equivalent).

In one embodiment, the affinity of specific binding is about 2 times greater than background binding, about 5 times greater than background binding, about 10 times greater than background binding, about 20 times greater than background binding, about 50 times greater than background binding, about 100 times greater than background binding, or about 1000 times greater than background binding or more.

A variety of assays are known for assessing binding affinity and/or determining whether a binding molecule (e.g., an antibody or fragment thereof) specifically binds to a particular ligand (e.g., an antigen, such as a BCMA protein). It is within the level of a skilled artisan to determine the binding affinity of a binding molecule, e.g., an antibody, for an antigen, e.g., BCMA. For example, in some embodiments, a BIAcore® instrument can be used to determine the binding kinetics and constants of a complex between two proteins (e.g., an antibody or fragment thereof, and an antigen, such as a BCMA cell surface protein, soluble BCMA protein), using surface plasmon resonance (SPR) analysis (see, e.g., Scatchard et al., Ann. N.Y. Acad. Sci. 51:660, 1949; Wilson, Science 295:2103, 2002; Wolff et al., Cancer Res. 53:2560, 1993; and U.S. Pat. Nos. 5,283,173, 5,468,614, or the equivalent).

SPR measures changes in the concentration of molecules at a sensor surface as molecules bind to or dissociate from the surface. The change in the SPR signal is directly proportional to the change in mass concentration close to the surface, thereby allowing measurement of binding kinetics between two molecules. The dissociation constant for the complex can be determined by monitoring changes in the refractive index with respect to time as buffer is passed over the chip. Other suitable assays for measuring the binding of one protein to another include, for example, immunoassays such as enzyme linked immunosorbent assays (ELISA) and radioimmunoassays (RIA), or determination of binding by monitoring the change in the spectroscopic or optical properties of the proteins through fluorescence, UV absorption, circular dichroism, or nuclear magnetic resonance (NMR). Other exemplary assays include, but are not limited to, Western blot, ELISA, analytical ultracentrifugation, spectroscopy, flow cytometry, sequencing and other methods for detection of expressed polynucleotides or binding of proteins.

In particular embodiments, the extracellular binding domain of a CAR comprises an antibody or antigen binding fragment thereof. An “antibody” refers to a binding agent that is a polypeptide comprising at least a light chain or heavy chain immunoglobulin variable region which specifically recognizes and binds an epitope of an antigen, such as a peptide, lipid, polysaccharide, or nucleic acid containing an antigenic determinant, such as those recognized by an immune cell.

An “antigen (Ag)” refers to a compound, composition, or substance that can stimulate the production of antibodies or a T cell response in an animal, including compositions (such as one that includes a cancer-specific protein) that are injected or absorbed into an animal. An antigen reacts with the products of specific humoral or cellular immunity, including those induced by heterologous antigens, such as the disclosed antigens. In particular embodiments, the target antigen is an epitope of a BCMA polypeptide.

An “epitope” or “antigenic determinant” refers to the region of an antigen to which a binding agent binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5, about 9, or about 8-10 amino acids in a unique spatial conformation.

Antibodies include antigen binding fragments thereof, such as Camel Ig, Ig NAR, Fab fragments, Fab′ fragments, F(ab)′2 fragments, F(ab)′3 fragments, Fv, single chain Fv proteins (“scFv”), bis-scFv, (scFv)₂, minibodies, diabodies, triabodies, tetrabodies, disulfide stabilized Fv proteins (“dsFv”), and single-domain antibody (sdAb, Nanobody) and portions of full length antibodies responsible for antigen binding. The term also includes genetically engineered forms such as chimeric antibodies (for example, humanized murine antibodies), heteroconjugate antibodies (such as, bispecific antibodies) and antigen binding fragments thereof. See also, Pierce Catalog and Handbook, 1994-1995 (Pierce Chemical Co., Rockford, IL); Kuby, J., Immunology, 3rd Ed., W. H. Freeman & Co., New York, 1997.

As would be understood by the skilled person and as described elsewhere herein, a complete antibody comprises two heavy chains and two light chains. Each heavy chain consists of a variable region and a first, second, and third constant region, while each light chain consists of a variable region and a constant region. Mammalian heavy chains are classified as α, δ, ε, γ, and μ. Mammalian light chains are classified as λ or κ. Immunoglobulins comprising the α, δ, ε, γ, and μ heavy chains are classified as immunoglobulin (Ig)A, IgD, IgE, IgG, and IgM. The complete antibody forms a “Y” shape. The stem of the Y consists of the second and third constant regions (and for IgE and IgM, the fourth constant region) of two heavy chains bound together and disulfide bonds (inter-chain) are formed in the hinge. Heavy chains γ, α and δ have a constant region composed of three tandem (in a line) Ig domains, and a hinge region for added flexibility; heavy chains and s have a constant region composed of four immunoglobulin domains. The second and third constant regions are referred to as “CH2 domain” and “CH3 domain”, respectively. Each arm of the Y includes the variable region and first constant region of a single heavy chain bound to the variable and constant regions of a single light chain. The variable regions of the light and heavy chains are responsible for antigen binding.

Light and heavy chain variable regions contain a “framework” region interrupted by three hypervariable regions, also called “complementarity-determining regions” or “CDRs.” The CDRs can be defined or identified by conventional methods, such as by sequence according to Kabat et al (Wu, T T and Kabat, E. A., J Exp Med. 132(2):211-50, (1970); Borden, P. and Kabat E. A., PNAS, 84: 2440-2443 (1987); (see, Kabat et al., Sequences of Proteins of Immunological Interest, U.S. Department of Health and Human Services, 1991, which is hereby incorporated by reference), or by structure according to Chothia et al (Chothia, C. and Lesk, A. M., J Mol. Biol., 196(4): 901-917 (1987), Chothia, C. et al, Nature, 342: 877-883 (1989)).

The sequences of the framework regions of different light or heavy chains are relatively conserved within a species, such as humans. The framework region of an antibody, that is the combined framework regions of the constituent light and heavy chains, serves to position and align the CDRs in three-dimensional space. The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, the CDRs located in the variable domain of the heavy chain of the antibody are referred to as CDRH1, CDRH2, and CDRH3, whereas the CDRs located in the variable domain of the light chain of the antibody are referred to as CDRL1, CDRL2, and CDRL3. Antibodies with different specificities (i.e., different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs). Illustrative examples of light chain CDRs that are suitable for constructing humanized BCMA CARS contemplated herein include, but are not limited to the CDR sequences set forth in SEQ ID NOs:1-3. Illustrative examples of heavy chain CDRs that are suitable for constructing humanized BCMA CARs contemplated herein include, but are not limited to the CDR sequences set forth in SEQ ID NOs:4-6.

References to “V_{H}” or “VH” refer to the variable region of an immunoglobulin heavy chain, including that of an antibody, Fv, scFv, dsFv, Fab, or other antibody fragment as disclosed herein. References to “V_{L}” or “VL” refer to the variable region of an immunoglobulin light chain, including that of an antibody, Fv, scFv, dsFv, Fab, or other antibody fragment as disclosed herein.

A “monoclonal antibody” is an antibody produced by a single clone of B lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

A “chimeric antibody” has framework residues from one species, such as human, and CDRs (which generally confer antigen binding) from another species, such as a mouse. In particular embodiments, a CAR contemplated herein comprises antigen-specific binding domain that is a chimeric antibody or antigen binding fragment thereof.

A “humanized” antibody is an immunoglobulin including a human framework region and one or more CDRs from a non-human (for example a mouse, rat, or synthetic) immunoglobulin. The non-human immunoglobulin providing the CDRs is termed a “donor,” and the human immunoglobulin providing the framework is termed an “acceptor.”

Also among the anti-BCMA antibodies included in the provided CARs are human antibodies. A “human antibody” is an antibody with an amino acid sequence corresponding to that of an antibody produced by a human or a human cell, or non-human source that utilizes human antibody repertoires or other human antibody-encoding sequences, including human antibody libraries. The term excludes humanized forms of non-human antibodies comprising non-human antigen-binding regions, such as those in which all or substantially all CDRs are non-human. The term includes antigen-binding fragments of human antibodies.

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic animals, the endogenous immunoglobulin loci have generally been inactivated. Human antibodies also may be derived from human antibody libraries, including phage display and cell-free libraries, containing antibody-encoding sequences derived from a human repertoire.

In particular embodiments, a murine anti-BCMA (e.g., human BCMA) antibody or antigen binding fragment thereof, includes but is not limited to a Camel Ig (a camelid antibody (VHH)), Ig NAR, Fab fragments, Fab′ fragments, F(ab)′₂ fragments, F(ab)′₃ fragments, Fv, single chain Fv antibody (“scFv”), bis-scFv, (scFv)₂, minibody, diabody, triabody, tetrabody, disulfide stabilized Fv protein (“dsFv”), and single-domain antibody (sdAb, Nanobody).

“Camel Ig” or “camelid VHH” as used herein refers to the smallest known antigen-binding unit of a heavy chain antibody (Koch-Nolte, et al, FASEB J., 21: 3490-3498 (2007)). A “heavy chain antibody” or a “camelid antibody” refers to an antibody that contains two VH domains and no light chains (Riechmann L. et al, J. Immunol. Methods 231:25-38 (1999); WO94/04678; WO94/25591; U.S. Pat. No. 6,005,079).

“IgNAR” of “immunoglobulin new antigen receptor” refers to class of antibodies from the shark immune repertoire that consist of homodimers of one variable new antigen receptor (VNAR) domain and five constant new antigen receptor (CNAR) domains. IgNARs represent some of the smallest known immunoglobulin-based protein scaffolds and are highly stable and possess efficient binding characteristics. The inherent stability can be attributed to both (i) the underlying Ig scaffold, which presents a considerable number of charged and hydrophilic surface exposed residues compared to the conventional antibody VH and VL domains found in murine antibodies; and (ii) stabilizing structural features in the complementary determining region (CDR) loops including inter-loop disulphide bridges, and patterns of intra-loop hydrogen bonds.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-combining sites and is still capable of cross-linking antigen.

“Fv” is the minimum antibody fragment which contains a complete antigen-binding site. In one embodiment, a two-chain Fv species consists of a dimer of one heavy- and one light-chain variable domain in tight, non-covalent association. In a single-chain Fv (scFv) species, one heavy- and one light-chain variable domain can be covalently linked by a flexible peptide linker such that the light and heavy chains can associate in a “dimeric” structure analogous to that in a two-chain Fv species. It is in this configuration that the three hypervariable regions (HVRs) of each variable domain interact to define an antigen-binding site on the surface of the VH-VL dimer. Collectively, the six HVRs confer antigen-binding specificity to the antibody. However, even a single variable domain (or half of an Fv comprising only three HVRs specific for an antigen) has the ability to recognize and bind antigen, although at a lower affinity than the entire binding site.

The Fab fragment contains the heavy- and light-chain variable domains and also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

The term “diabodies” refers to antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies may be bivalent or bispecific. Diabodies are described more fully in, for example, EP 404,097; WO 1993/01161; Hudson et al., Nat. Med. 9:129-134 (2003); and Hollinger et al., PNAS USA 90: 6444-6448 (1993). Triabodies and tetrabodies are also described in Hudson et al., Nat. Med. 9:129-134 (2003).

“Single domain antibody” or “sdAb” or “nanobody” refers to an antibody fragment that consists of the variable region of an antibody heavy chain (VH domain) or the variable region of an antibody light chain (VL domain) (Holt, L., et al, 2003, Trends in Biotechnology, 21(11): 484-490).

“Single-chain Fv” or “scFv” antibody fragments comprise the VH and VL domains of antibody, wherein these domains are present in a single polypeptide chain and in either orientation (e.g., VL-VH or VH-VL). Generally, the scFv polypeptide further comprises a polypeptide linker between the VH and VL domains which enables the scFv to form the desired structure for antigen binding. For a review of scFv, see, e.g., Pluckthun, in The Pharmacology of Monoclonal Antibodies, vol. 113, Rosenburg and Moore eds., (Springer-Verlag, New York, 1994), pp. 269-315.

In certain embodiments, a CAR contemplated herein comprises antigen-specific binding domain that is a murine scFv. Single chain antibodies may be cloned form the V region genes of a hybridoma specific for a desired target. The production of such hybridomas has become routine. A technique which can be used for cloning the variable region heavy chain (VH) and variable region light chain (VL) has been described, for example, in Orlandi et al., PNAS, 1989; 86: 3833-3837.

In some embodiments, the CAR includes a BCMA-binding portion or portions of the antibody molecule, such as a heavy chain variable (V_{H}) region and/or light chain variable (V_{L}) region of the antibody, e.g., an scFv antibody fragment. The chimeric receptors, such as CARs, generally include an extracellular antigen binding domain, such as a portion of an antibody molecule, generally a variable heavy (VH) chain region and/or variable light (VL) chain region of the antibody, e.g., an scFv antibody fragment. In some embodiments, the provided BCMA-binding CARs contain an antibody, such as an anti-BCMA antibody, or an antigen-binding fragment thereof that confers the BCMA-binding properties of the provided CAR. In some embodiments, the antibody or antigen-binding domain can be any anti-BCMA antibody described or derived from any anti-BCMA antibody described. See, e.g., Carpenter et al., Clin. Cancer Res., 2013, 19(8):2048-2060; Feng et al., Scand. J. Immunol. (2020) 92:e12910; U.S. Pat. Nos. 9,034,324 9,765,342; U.S. Patent publication No. US2016/0046724, US20170183418; and International published PCT App. No. WO 2016090320, WO2016090327, WO2016094304, WO2016014565, WO2016014789, WO2010104949, WO2017025038, WO2017173256, WO2018085690, or WO2021091978. Any of such anti-BCMA antibodies or antigen-binding fragments can be used in the provided CARs. In some embodiments, the anti-BCMA CAR contains one or more single-domain anti-BCMA antibodies. In some embodiments, the one or more single-domain anti-BCMA antibodies is derived from an antibody described in WO2017025038 or WO2018028647. In some embodiments, the anti-BCMA CAR comprises the single-domain antibody sequence set forth in SEQ ID NO:111. In some embodiments, the anti-BCMA CAR contains two single-domain anti-BCMA antibodies. In some embodiments, the two single-domain anti-BCMA antibodies are derived from one or more antibodies described in WO2017025038 or WO2018028647. In some embodiments, the BCMA binding domain comprises or consists of A37353-G4S-A37917 (G4S being a linker between the two binding domains), described in WO2017025038 or WO2018028647, and provided, e.g., in SEQ ID NOs:300, 301 and 302 of WO2017025038 or WO2018028647 (with or without signal peptide). In some embodiments, the anti-BCMA CAR contains an antigen-binding domain that is an scFv containing a variable heavy (V_{H}) and/or a variable light (V_{L}) region. In some embodiments, the scFv containing a variable heavy (V_{H}) and/or a variable light (V_{L}) region is derived from an antibody described in WO2016090320 or WO2016090327. In some embodiments, the scFv containing a variable heavy (V_{H}) and/or a variable light (V_{L}) region is derived from an antibody described in WO 2019/090003. In some embodiments, the scFv containing a variable heavy (V_{H}) and/or a variable light (V_{L}) region is derived from an antibody described in WO2016094304 or WO2021091978. In some embodiments, the scFv containing a variable heavy (V_{H}) and/or a variable light (V_{L}) region is derived from an antibody described in WO2018133877. In some embodiments, the scFv containing a variable heavy (V_{H}) and/or a variable light (V_{L}) region is derived from an antibody described in WO2019149269. In some embodiments, the anti-BCMA CAR is any as described in WO2019173636 or WO2020051374A. In some embodiments, the anti-BCMA CAR is any as described in WO2018102752. In some embodiments, the anti-BCMA CAR is any as described in WO2020112796 or WO2021173630.

In some embodiments, the antibody, e.g., the anti-BCMA antibody or antigen-binding fragment, contains a heavy and/or light chain variable (V_{H} or V_{L}) region sequence as described, or a sufficient antigen-binding portion thereof. In some embodiments, the anti-BCMA antibody, e.g., antigen-binding fragment, contains a V_{H} region sequence or sufficient antigen-binding portion thereof that contains a CDR-H1, CDR-H2 and/or CDR-H3 as described. In some embodiments, the anti-BCMA antibody, e.g., antigen-binding fragment, contains a V_{L} region sequence or sufficient antigen-binding portion that contains a CDR-L1, CDR-L2 and/or CDR-L3 as described. In some embodiments, the anti-BCMA antibody, e.g., antigen-binding fragment, contains a V_{H} region sequence that contains a CDR-H1, CDR-H2 and/or CDR-H3 as described and contains a V_{L} region sequence that contains a CDR-L1, CDR-L2 and/or CDR-L3 as described. Also among the antibodies are those having sequences at least at or about 90%, about 91%, about 92%, about 93%, about 94%, about 95%, about 96%, about 97%, about 98%, or about 99% identical to such a sequence.

In some embodiments, the antibody is a single domain antibody (sdAb) comprising only a V_{H} region sequence or a sufficient antigen-binding portion thereof, such as any of the above described V_{H} sequences (e.g., a CDR-H1, a CDR-H2, a CDR-H3 and/or a CDR-H4).

In some embodiments, an antibody provided herein (e.g., an anti-BCMA antibody) or antigen-binding fragment thereof comprising a V_{H} region further comprises a light chain or a sufficient antigen binding portion thereof. For example, in some embodiments, the antibody or antigen-binding fragment thereof contains a V_{H} region and a V_{L} region, or a sufficient antigen-binding portion of a V_{H} and V_{L} region. In such embodiments, a V_{H} region sequence can be any of the above described V_{H} sequence. In some such embodiments, the antibody is an antigen-binding fragment, such as a Fab or an scFv. In some such embodiments, the antibody is a full-length antibody that also contains a constant region.

In some embodiments, the CAR is an anti-BCMA CAR that is specific for BCMA, e.g. human BCMA. Chimeric antigen receptors containing anti-BCMA antibodies, including mouse anti-human BCMA antibodies and human anti-human BCMA antibodies, and cells expressing such chimeric receptors have been previously described. See Carpenter et al., Clin Cancer Res., 2013, 19(8):2048-2060, U.S. Pat. No. 9,765,342, WO 2016/090320, WO2016090327, WO2010104949A2, WO2016/0046724, WO2016/014789, WO2016/094304, WO2017/025038, and WO2017173256.

In some embodiments, the anti-BCMA CAR contains an antigen-binding domain, such as an scFv, containing a variable heavy (V_{H}) and/or a variable light (V_{L}) region derived from an antibody described in WO2016094304 or WO2021091978. In some embodiments, the antigen-binding domain is an antibody fragment containing a variable heavy chain (VH) and a variable light chain (VL) region. In some embodiments, the anti-BCMA CAR contains an antigen-binding domain, such as an scFv, containing a variable heavy (V_{H}) and/or a variable light (V_{L}) region derived from an antibody described in WO 2016/090320 or WO2016090327.

In some embodiments, the antigen-binding domain is an antibody fragment containing a variable heavy chain (V_{H}) and a variable light chain (V_{L}) region. In some aspects, the V_{H} region is or includes an amino acid sequence having at least 90%, 91%, 92%, 93%, 94%, 95%, 96%, 97%, 98% or 99% sequence identity to the V_{H} region amino acid sequence set forth in any of SEQ ID NOs:8, 56, 58, 60, 66, 68, 70, 75, 77, 79, 81, 83, 85, 87, 89, 91, 93, 95, 97, 99, 101, 103, 105, 107, 109, 178, 180, 182 and 184; and/or the V_{L} region is or includes an amino acid sequence having at least 90%, 91%, 92%, 93%, 94%, 95%, 96%, 97%, 98%, or 99% sequence identity to the V_{L} region amino acid sequence set forth in any of SEQ ID NOs:7, 57, 59, 61, 67, 69, 71, 76, 78, 80, 82, 84, 86, 88, 90, 92, 94, 96, 98, 100, 102, 104, 106, 108, 110, 179, 181, 183 and 185.

In some embodiments, the antigen-binding domain, such as an scFv, contains a V_{H} set forth in SEQ ID NO:8 and a V_{L} set forth in SEQ ID NO:7. In some embodiments, the antigen-binding domain, such as an scFv, contains a V_{H} set forth in SEQ ID NO:56 and a V_{L} set forth in SEQ ID NO:57. In some embodiments, the antigen-binding domain, such as an scFv, contains a V_{H} set forth in SEQ ID NO:58 and a V_{L} set forth in SEQ ID NO:59. In some embodiments, the antigen-binding domain, such as an scFv, contains a V_{H} set forth in SEQ ID NO:60 and a V_{L} set forth in SEQ ID NO:61. In some embodiment the antigen-binding domain, such as an scFv, contains a V_{H} set forth in SEQ ID NO:66 and a V_{L} set forth in SEQ ID NO:67. In some embodiments, the antigen-binding domain, such as an scFv, contains a V_{H} set forth in SEQ ID NO:68 and a V_{L} set forth in SEQ ID NO:69. In some embodiments, the antigen-binding domain, such as an scFv, contains a V_{H} set forth in SEQ ID NO:70 and a V_{L} set forth in SEQ ID NO:71. In some embodiments, the antigen-binding domain, such as an scFv, contains a V_{H} set forth in SEQ ID NO:75 and a V_{L} set forth in SEQ ID NO:76. In some embodiments, the antigen-binding domain, such as an scFv, contains a V_{H} set forth in SEQ ID NO:77 and a V_{L} set forth in SEQ ID NO:78. In some embodiments, the antigen-binding domain, such as an scFv, contains a V_{H} set forth in SEQ ID NO:79 and a V_{L} set forth in SEQ ID NO:80. In some embodiments, the antigen-binding domain, such as an scFv, contains a V_{H} set forth in SEQ ID NO:81 and a V_{L} set forth in SEQ ID NO:82. In some embodiments, the antigen-binding domain, such as an scFv, contains a V_{H} set forth in SEQ ID NO:83 and a V_{L} set forth in SEQ ID NO:84. In some embodiments, the antigen-binding domain, such as an scFv, contains a V_{H} set forth in SEQ ID NO:85 and a V_{L} set forth in SEQ ID NO:86. In some embodiments, the antigen-binding domain, such as an scFv, contains a V_{H} set forth in SEQ ID NO:87 and a V_{L} set forth in SEQ ID NO:88. In some embodiments, the antigen-binding domain, such as an scFv, contains a V_{H} set forth in SEQ ID NO:89 and a V_{L} set forth in SEQ ID NO:90. In some embodiments, the antigen-binding domain, such as an scFv, contains a V_{H} set forth in SEQ ID NO:91 and a V_{L} set forth in SEQ ID NO:92. In some embodiments, the antigen-binding domain, such as an scFv, contains a V_{H} set forth in SEQ ID NO:93 and a V_{L} set forth in SEQ ID NO:94. In some embodiments, the antigen-binding domain, such as an scFv, contains a V_{H} set forth in SEQ ID NO:95 and a V_{L} set forth in SEQ ID NO:96. In some embodiments, the antigen-binding domain, such as an scFv, contains a V_{H} set forth in SEQ ID NO:97 and a V_{L} set forth in SEQ ID NO:98. In some embodiments, the antigen-binding domain, such as an scFv, contains a V_{H} set forth in SEQ ID NO:99 and a V_{L} set forth in SEQ ID NO: 100. In some embodiments, the antigen-binding domain, such as an scFv, contains a V_{H} set forth in SEQ ID NO:101 and a V_{L} set forth in SEQ ID NO: 102. In some embodiments, the antigen-binding domain, such as an scFv, contains a V_{H} set forth in SEQ ID NO: 103 and a V_{L} set forth in SEQ ID NO: 104. In some embodiments, the antigen-binding domain, such as an scFv, contains a V_{H} set forth in SEQ ID NO: 105 and a V_{L} set forth in SEQ ID NO: 106. In some embodiments, the antigen-binding domain, such as an scFv, contains a V_{H} set forth in SEQ ID NO: 107 and a V_{L} set forth in SEQ ID NO: 108. In some embodiments, the antigen-binding domain, such as an scFv, contains a V_{H} set forth in SEQ ID NO: 109 and a V_{L} set forth in SEQ ID NO: 110. In some embodiments, the antigen-binding domain, such as an scFv, contains a V_{H} set forth in SEQ ID NO: 178 and a V_{L} set forth in SEQ ID NO: 179. In some embodiments, the antigen-binding domain, such as an scFv, contains a V_{H} set forth in SEQ ID NO: 180 and a V_{L} set forth in SEQ ID NO:181. In some embodiments, the antigen-binding domain, such as an scFv, contains a V_{H} set forth in SEQ ID NO: 182 and a V_{L} set forth in SEQ ID NO: 183. In some embodiments, the antigen-binding domain, such as an scFv, contains a V_{H} set forth in SEQ ID NO: 184 and a V_{L} set forth in SEQ ID NO: 185. In some embodiments, the V_{H} or V_{L} has a sequence of amino acids that exhibits at least 85%, 86%, 87%, 88%, 89%, 90%, 91%, 92%, 93%, 94%, 95%, 96%, 97%, 98%, 99% or more sequence identity to any of the foregoing V_{H} or V_{L} sequences, and retains binding to BCMA. In some embodiments, the V_{H} region is amino-terminal to the V_{L} region. In some embodiments, the V_{H} region is carboxy-terminal to the V_{L} region. In some embodiments, the variable heavy and variable light chains are connected by a linker. In some embodiments, the linker is set forth in SEQ ID NOs:63, 22, 64, or 72. In some embodiments, the linker is set forth in SEQ ID NOs:54 or 55.

Among a provided anti-BCMA CAR is a CAR in which the antibody or antigen-binding fragment contains a V_{H} region comprising the sequence set forth in SEQ ID NO:8 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:8; and contains a V_{L} region comprising the sequence set forth in SEQ ID NO:7 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:7. In some embodiments, the antibody or antigen-binding fragment of the provided CAR contains a V_{H} region that has a CDRH1, a CDRH2 and a CDRH3 comprising the amino acid sequence of SEQ ID NOs:4, 5, and 6, respectively and a V_{L} region that has a CDRL1, a CDRL2 and a CDRL3 comprising the amino acid sequence of SEQ ID NOs: 1, 2, and 3, respectively. In some embodiments, the antibody or antigen-binding fragment of the provided CAR contains a V_{H} region that has a CDRH1, a CDRH2 and a CDRH3 comprising the amino acid sequence of SEQ ID NOs:222, 223, and 224, respectively and a V_{L} region that has a CDRL1, a CDRL2 and a CDRL3 comprising the amino acid sequence of SEQ ID NOs:225, 226, and 227, respectively. In some embodiments, the antibody or antigen-binding fragment of the provided CAR contains a V_{H} region that has a CDRH1, a CDRH2 and a CDRH3 comprising the amino acid sequence of SEQ ID NOs:228, 229, and 230, respectively and a V_{L} region that has a CDRL1, a CDRL2 and a CDRL3 comprising the amino acid sequence of SEQ ID NOs:231, 232, and 233, respectively. In some embodiments, the antibody or antigen-binding fragment of the provided CAR contains a V_{H} region that has a CDRH1, a CDRH2 and a CDRH3 comprising the amino acid sequence of SEQ ID NOs:234, 235, and 236, respectively and a V_{L} region that has a CDRL1, a CDRL2 and a CDRL3 comprising the amino acid sequence of SEQ ID NOs:237, 238, and 239, respectively. In some embodiments, the V_{H} region comprises the sequence set forth in SEQ ID NO:8 and the V_{L} region comprises the sequence set forth in SEQ ID NO:7. In some embodiments, the antibody or antigen-binding fragment is a single-chain antibody fragment, such as an scFv. In some embodiments, the scFv comprises the sequence of amino acids set forth in SEQ ID NO:38 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:38. In some embodiments, the anti-BCMA CAR has the sequence of amino acids set forth in SEQ NO:37 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:37. In some embodiments, the anti-BCMA CAR is encoded by the polynucleotide sequence set forth in SEQ NO:240 or a polynucleotide sequence of at least at or about 90%, at or about 91%, at or about 92%, at or about 93%, at or about 94%, at or about 95%, at or about 96%, at or about 97%, at or about 98%, or at or about 99% identity to SEQ ID NO:240.

Among a provided anti-BCMA CAR is a CAR in which the antibody or antigen-binding fragment contains a V_{H} region comprising the sequence set forth in SEQ ID NO:60 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:60; and contains a V_{L} region comprising the sequence set forth in SEQ ID NO:61 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO:61. In some embodiments, the antibody or antigen-binding fragment of the provided CAR contains a V_{H} region that has a CDRH1, a CDRH2 and a CDRH3 comprising the amino acid sequence of SEQ ID NOs:206, 207, and 208, respectively and a V_{L} region that has a CDRL1, a CDRL2 and a CDRL3 comprising the amino acid sequence of SEQ ID NOs:216, 217 and 218, respectively. In some embodiments, the antibody or antigen-binding fragment of the provided CAR contains a V_{H} region that has a CDRH1, a CDRH2 and a CDRH3 comprising the amino acid sequence of SEQ ID NOs:209, 210, and 215, respectively and a V_{L} region that has a CDRL1, a CDRL2 and a CDRL3 comprising the amino acid sequence of SEQ ID NOs:216, 217, and 218, respectively. In some embodiments, the antibody or antigen-binding fragment of the provided CAR contains a V_{H} region that has a CDRH1, a CDRH2 and a CDRH3 comprising the amino acid sequence of SEQ ID NOs:211, 212, and 215, respectively and a V_{L} region that has a CDRL1, a CDRL2 and a CDRL3 comprising the amino acid sequence of SEQ ID NOs:216, 217, and 218, respectively. In some embodiments, the antibody or antigen-binding fragment of the provided CAR contains a V_{H} region that has a CDRH1, a CDRH2 and a CDRH3 comprising the amino acid sequence of SEQ ID NOs:213, 214, and 215. In some embodiments, the antibody or antigen-binding fragment of the provided CAR contains a V_{H} region that has a CDRH1, a CDRH2 and a CDRH3 comprising the amino acid sequence of SEQ ID NOs:213, 214, and 215, respectively and a V_{L} region that has a CDRL1, a CDRL2 and a CDRL3 comprising the amino acid sequence of SEQ ID NOs:219, 220, and 218, respectively. In some embodiments, the V_{H} region comprises the sequence set forth in SEQ ID NO:60 and the V_{L} region comprises the sequence set forth in SEQ ID NO:61. In some embodiments, the antibody or antigen-binding fragment is a single-chain antibody fragment, such as an scFv. In some embodiments, the scFv comprises the sequence of amino acids set forth in SEQ ID NO:221 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:221. In some embodiments, the anti-BCMA CAR has the sequence of amino acids set forth in SEQ NO:157 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO: 157. In some embodiments, the anti-BCMA CAR has the sequence of amino acids set forth in SEQ NO:158 or a sequence of amino acids at least at or about 90%, at or about 91%, at or about 92%, at or about 93%, at or about 94%, at or about 95%, at or about 96%, at or about 97%, at or about 98%, or at or about 99% identity to SEQ ID NO:158.

In some embodiments, the scFv comprises the amino acid sequence set forth in any one of SEQ ID NOs:241-272, or an amino acid sequence having at least 90, 95, 96, 97, 98, 99, or 100% sequence identity to a sequence set forth in any one of SEQ ID NOs:241-272.

In some embodiments, the antigen-binding domain comprises an sdAb. In some embodiments, the antigen-binding domain contains the sequence set forth by SEQ ID NO:77. In some embodiments, the antigen-binding domain comprises a sequence at least or about 50, 60, 70, 80, 85, 90, 95, 96, 97, 98, 99, or 100% identical to the sequence set forth by SEQ ID NO:77.

In some embodiments, the CAR comprises the amino acid sequence set forth in any one of SEQ ID NOs: 37 and 124-174, or an amino acid sequence having at least 90, 95, 96, 97, 98, or 99% sequence identity to a sequence set forth in any one of SEQ ID NOs:37 and 124-174.

In particular embodiments, the antigen-specific binding domain that is a murine scFv that binds a human BCMA polypeptide. Illustrative examples of variable heavy chains that are suitable for constructing BCMA CARs contemplated herein include, but are not limited to the amino acid sequences set forth in SEQ ID NO:8. Illustrative examples of variable light chains that are suitable for constructing BCMA CARs contemplated herein include, but are not limited to the amino acid sequences set forth in SEQ ID NO:7.

BCMA-specific binding domains provided herein also comprise one, two, three, four, five, or six CDRs. Such CDRs may be nonhuman CDRs or altered nonhuman CDRs selected from CDRL1, CDRL2 and CDRL3 of the light chain and CDRH1, CDRH2 and CDRH3 of the heavy chain. In certain embodiments, a BCMA-specific binding domain comprises (a) a light chain variable region that comprises a light chain CDRL1, a light chain CDRL2, and a light chain CDRL3, and (b) a heavy chain variable region that comprises a heavy chain CDRH1, a heavy chain CDRH2, and a heavy chain CDRH3.

### 2. Linkers

In certain embodiments, the CARs contemplated herein may comprise linker residues between the various domains, e.g., added for appropriate spacing and conformation of the molecule. In particular embodiments, the linker is a variable region linking sequence. A “variable region linking sequence” is an amino acid sequence that connects the V_{H} and V_{L} domains and provides a spacer function compatible with interaction of the two sub-binding domains so that the resulting polypeptide retains a specific binding affinity to the same target molecule as an antibody that comprises the same light and heavy chain variable regions. CARs contemplated herein, may comprise one, two, three, four, or five or more linkers. In particular embodiments, the length of a linker is about 1 to about 25 amino acids, about 5 to about 20 amino acids, or about 10 to about 20 amino acids, or any intervening length of amino acids. In some embodiments, the linker is 1, 2, 3, 4, 5, 6, 7, 8, 9, 10, 11, 12, 13, 14, 15, 16, 17, 18, 19, 20, 21, 22, 23, 24, 25, or more amino acids long.

Illustrative examples of linkers include glycine polymers (G)ₙ; glycine-serine polymers (G₁₋₅S₁₋₅)ₙ, where n is an integer of at least one, two, three, four, or five; glycine-alanine polymers; alanine-serine polymers; and other flexible linkers known in the art. Glycine and glycine-serine polymers are relatively unstructured, and therefore may be able to serve as a neutral tether between domains of fusion proteins such as the CARs described herein. Glycine accesses significantly more phi-psi space than even alanine, and is much less restricted than residues with longer side chains (see Scheraga, Rev. Computational Chem. 11173-142 (1992)). The ordinarily skilled artisan will recognize that design of a CAR in particular embodiments can include linkers that are all or partially flexible, such that the linker can include a flexible linker as well as one or more portions that confer less flexible structure to provide for a desired CAR structure.

Other exemplary linkers include, but are not limited to the following amino acid sequences: GGG; DGGGS (SEQ ID NO:12); TGEKP (SEQ ID NO:13) (see, e.g., Liu et al., PNAS 5525-5530 (1997)); GGRR (SEQ ID NO: 14) (Pomerantz et al. 1995, supra); (GGGGS)ₙ wherein n=1, 2, 3, 4 or 5, and where GGGGS is identified as SEQ ID NO: 15 (Kim et al., PNAS 93, 1156-1160 (1996.); EGKSSGSGSESKVD (SEQ ID NO:16) (Chaudhary et al., 1990, Proc. Natl. Acad. Sci. U.S.A. 87:1066-1070); KESGSVSSEQLAQFRSLD (SEQ ID NO:17) (Bird et al., 1988, Science 242:423-426), GGRRGGGS (SEQ ID NO: 18); LRQRDGERP (SEQ ID NO: 19); LRQKDGGGSERP (SEQ ID NO:20); LRQKd(GGGS)₂ ERP (SEQ ID NO:21). Alternatively, flexible linkers can be rationally designed using a computer program capable of modeling both DNA-binding sites and the peptides themselves (Desjarlais & Berg, PNAS 90:2256-2260 (1993), PNAS 91:11099-11103 (1994) or by phage display methods. In one embodiment, the linker comprises the following amino acid sequence: GSTSGSGKPGSGEGSTKG (SEQ ID NO:22) (Cooper et al., Blood, 101(4): 1637-1644 (2003)).

In some embodiments, the antibody is an antigen-binding fragment, such as a scFv, that includes one or more linkers joining two antibody domains or regions, such as a heavy chain variable (V_{H}) region and a light chain variable (V_{L}) region. The linker typically is a peptide linker, e.g., a flexible and/or soluble peptide linker. Among the linkers are those rich in glycine and serine and/or in some cases threonine. In some embodiments, the linkers further include charged residues such as lysine and/or glutamate, which can improve solubility. In some embodiments, the linkers further include one or more proline. In some aspects, the linkers rich in glycine and serine (and/or threonine) include at least 80%, 85%, 90%, 91%, 92%, 93%, 94%, 95%, 96%, 97%, 98%, or 99% such amino acid(s). In some embodiments, they include at least at or about 50%, 55%, 60%, 70%, or 75%, glycine, serine, and/or threonine. In some embodiments, the linker is comprised substantially entirely of glycine, serine, and/or threonine. The linkers generally are between about 5 and about 50 amino acids in length, typically between at or about 10 and at or about 30, e.g., 10, 11, 12, 13, 14, 15, 16, 17, 18, 19, 20, 21, 22, 23, 24, 25, 26, 27, 28, 29, or 30, and in some examples between 10 and 25 amino acids in length. Exemplary linkers include linkers having various numbers of repeats of the sequence GGGGS (4GS; SEQ ID NO: 15) or GGGS (3GS; SEQ ID NO:62), such as between 2, 3, 4, and 5 repeats of such a sequence. Exemplary linkers include those having or consisting of an sequence set forth in SEQ ID NO:63 (GGGGSGGGGSGGGGS), SEQ ID NO:22 (GSTSGSGKPGSGEGSTKG), SEQ ID NO:64 (SRGGGGSGGGGSGGGGSLEMA), or SEQ ID NO:72 (ASGGGGSGGRASGGGGS). In some embodiments, the linker is or comprises the sequence set forth in SEQ ID NO:22. In some embodiments, the linker is or comprises the sequence set forth in SEQ ID NO:274.

### 3. Spacer Domain

In particular embodiments, the binding domain of the CAR is followed by one or more “spacer domains,” which refers to the region that moves the antigen binding domain away from the effector cell surface to enable proper cell/cell contact, antigen binding and activation (Patel et al., Gene Therapy, 1999; 6: 412-419). The spacer domain may be derived either from a natural, synthetic, semi-synthetic, or recombinant source. In certain embodiments, a spacer domain is a portion of an immunoglobulin, including, but not limited to, one or more heavy chain constant regions, e.g., CH2 and CH3. The spacer domain can include the amino acid sequence of a naturally occurring immunoglobulin hinge region or an altered immunoglobulin hinge region.

In some embodiments, the antibody portion of the recombinant receptor, e.g., CAR, further includes a spacer, which may be or include at least a portion of an immunoglobulin constant region or variant or modified version thereof, such as a hinge region, e.g., an IgG4 hinge region, an IgG1 hinge region, a C_{H}1/C_{L}, and/or Fc region. In one embodiment, the spacer domain comprises the CH2 and CH3 domains of IgG1 or IgG4. In some embodiments, the recombinant receptor further comprises a spacer and/or a hinge region. In some embodiments, the constant region or portion is of a human IgG, such as IgG4 or IgG1. In some aspects, the portion of the constant region serves as a spacer region between the antigen-recognition component, e.g., scFv, and transmembrane domain.

The binding domain of the CAR is generally followed by one or more “hinge domains,” which play a role in positioning the antigen binding domain away from the effector cell surface to enable proper cell/cell contact, antigen binding and activation. A CAR generally comprises one or more hinge domains between the binding domain and the transmembrane domain (TM). The hinge domain may be derived either from a natural, synthetic, semi-synthetic, or recombinant source. The hinge domain can include the amino acid sequence of a naturally occurring immunoglobulin hinge region or an altered immunoglobulin hinge region.

An “altered hinge region” refers to (a) a naturally occurring hinge region with up to 30% amino acid changes (e.g., up to 25%, 20%, 15%, 10%, or 5% amino acid substitutions or deletions), (b) a portion of a naturally occurring hinge region that is at least 10 amino acids (e.g., at least 12, 13, 14 or 15 amino acids) in length with up to 30% amino acid changes (e.g., up to 25%, 20%, 15%, 10%, or 5% amino acid substitutions or deletions), or (c) a portion of a naturally occurring hinge region that comprises the core hinge region (which may be 4, 5, 6, 7, 8, 9, 10, 11, 12, 13, 14, or 15, or at least 4, 5, 6, 7, 8, 9, 10, 11, 12, 13, 14, or 15 amino acids in length). In certain embodiments, one or more cysteine residues in a naturally occurring immunoglobulin hinge region may be substituted by one or more other amino acid residues (e.g., one or more serine residues). An altered immunoglobulin hinge region may alternatively or additionally have a proline residue of a wild type immunoglobulin hinge region substituted by another amino acid residue (e.g., a serine residue).

Other illustrative hinge domains suitable for use in the CARs described herein include the hinge region derived from the extracellular regions of type 1 membrane proteins such as CD8α, CD4, CD28 and CD7, which may be wild-type hinge regions from these molecules or may be altered. In certain embodiments, the hinge domain comprises a CD8α hinge region.

The spacer can be of a length that provides for increased responsiveness of the cell following antigen binding, as compared to in the absence of the spacer. Exemplary spacers, e.g., hinge regions, include those described in international patent application publication number WO2014031687. In some examples, the spacer is or is about 12 amino acids in length or is no more than 12 amino acids in length. Exemplary spacers include those having at least about 10 to 229 amino acids, about 10 to 200 amino acids, about 10 to 175 amino acids, about 10 to 150 amino acids, about 10 to 125 amino acids, about 10 to 100 amino acids, about 10 to 75 amino acids, about 10 to 50 amino acids, about 10 to 40 amino acids, about 10 to 30 amino acids, about 10 to 20 amino acids, or about 10 to 15 amino acids, and including any integer between the endpoints of any of the listed ranges. In some embodiments, a spacer region has about 12 amino acids or less, about 119 amino acids or less, or about 229 amino acids or less. In some embodiments, the spacer is a spacer having at least a particular length, such as having a length that is at least 100 amino acids, such as at least 110, 125, 130, 135, 140, 145, 150, 160, 170, 180, 190, 200, 210, 220, 230, 240, or 250 amino acids in length. Exemplary spacers include IgG4 hinge alone, IgG4 hinge linked to C_{H}2 and C_{H}3 domains, or IgG4 hinge linked to the C_{H}3 domain. Exemplary spacers include IgG4 hinge alone, IgG4 hinge linked to C_{H}2 and C_{H}3 domains, or IgG4 hinge linked to the C_{H}3 domain. Exemplary spacers include IgG4 hinge alone, IgG4 hinge linked to C_{H}2 and C_{H}3 domains, or IgG4 hinge linked to the C_{H}3 domain. Exemplary spacers include, but are not limited to, those described in Hudecek et al., Clin. Cancer Res., 19:3153 (2013), Hudecek et al. (2015) Cancer Immunol Res. 3(2): 125-135, international patent application publication number WO2014031687, U.S. Pat. No. 8,822,647 or published app. No. US2014/0271635. In some embodiments, the spacer includes a sequence of an immunoglobulin hinge region, a C_{H}2 and C_{H}3 region. In some embodiments, one of more of the hinge, C_{H}2 and C_{H}3 is derived all or in part from IgG4 or IgG2. In some cases, the hinge, C_{H}2 and C_{H}3 is derived from IgG4. In some aspects, one or more of the hinge, C_{H}2 and C_{H}3 is chimeric and contains sequence derived from IgG4 and IgG2. In some examples, the spacer contains an IgG4/2 chimeric hinge, an IgG2/4 C_{H}2, and an IgG4 C_{H}3 region.

In some embodiments, the spacer can be derived all or in part from IgG4 and/or IgG2 and can contain mutations, such as one or more single amino acid mutations in one or more domains. In some examples, the amino acid modification is a substitution of a proline (P) for a serine (S) in the hinge region of an IgG4. In some embodiments, the amino acid modification is a substitution of a glutamine (Q) for an asparagine (N) to reduce glycosylation heterogeneity, such as an N177Q mutation at position 177, in the C_{H}2 region, of the full-length IgG4 Fc sequence or an N176Q at position 176, in the C_{H}2 region, of the full-length IgG4 Fc sequence.

In some embodiments, the spacer has the sequence ESKYGPPCPPCP (set forth in SEQ ID NO:39), and is encoded by the sequence set forth in SEQ ID NO:40. In some embodiments, the spacer has the sequence set forth in SEQ ID NO:41. In some embodiments, the spacer has the sequence set forth in SEQ ID NO:42. In some embodiments, the encoded spacer is or contains the sequence set forth in SEQ ID NO:65. In some embodiments, the constant region or portion is of IgD. In some embodiments, the spacer has the sequence set forth in SEQ ID NO:43. In some embodiments, the spacer has the sequence set forth in SEQ ID NO: 123.

Other exemplary spacer regions include hinge regions derived from CD8a, CD28, CTLA4, PD-1, or FcγRIIIa. In some embodiments, the spacer contains a truncated extracellular domain or hinge region of a CD8a, CD28, CTLA4, PD-1, or FcγRIIIa. In some embodiments, the spacer is a truncated CD28 hinge region. In some embodiments, a short oligo- or polypeptide linker, for example, a linker of between 2 and 10 amino acids in length, such as one containing alanines or alanine and arginine, e.g., alanine triplet (AAA; SEQ ID NO:274) or RAAA (SEQ ID NO: 177), is present and forms a linkage between the scFv and the spacer region of the CAR. In some embodiments, the spacer has the sequence set forth in SEQ ID NO: 112. In some embodiments, the spacer has the sequence set forth in SEQ ID NO: 114. In some embodiments, the spacer has the sequence set forth in any of SEQ ID NOs:115-117. In some embodiments, the spacer has the sequence set forth in SEQ ID NO: 116. In some embodiments, the spacer has the sequence set forth in SEQ ID NO: 118. In some embodiments, the spacer has the sequence set forth in SEQ ID NO: 120. In some embodiments, the spacer has the sequence set forth in SEQ ID NO: 122.

In some embodiments, the spacer has a sequence of amino acids that exhibits at least 85%, 86%, 87%, 88%, 89%, 90%, 91%, 92%, 93%, 94%, 95%, 96%, 97%, 98%, 99% or more sequence identity to any of SEQ ID NOs:39, 41, 42, 43, 65, 112, 114, 115, 116, 117, 118, 120, 122, or 123.

In some embodiments, the spacer has the sequence set forth in SEQ ID NOs:190-198. In some embodiments, the spacer has a sequence of amino acids that exhibits at least 85%, 86%, 87%, 88%, 89%, 90%, 91%, 92%, 93%, 94%, 95%, 96%, 97%, 98%, 99% or more sequence identity to any of SEQ ID NOs:190-198.

### 4. Transmembrane Domain

The antigen binding domain generally is linked to one or more intracellular signaling components, such as signaling components that mimic stimulation and/or activation through an antigen receptor complex, such as a TCR complex, in the case of a CAR, and/or signal via another cell surface receptor. Thus, in some embodiments, the antigen-binding component (e.g., antibody) is linked to one or more transmembrane and intracellular signaling domains. In some embodiments, the chimeric antigen receptor includes a transmembrane domain linking the extracellular domain and the intracellular signaling domain. The transmembrane (TM) domain is the portion of the CAR that fuses the extracellular binding portion and intracellular signaling domain and anchors the CAR to the plasma membrane of the immune effector cell. In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

The TM domain may be derived either from a natural, synthetic, semi-synthetic, or recombinant source. The TM domain may be derived from (i.e., comprise at least the transmembrane region(s) of) the alpha, beta or zeta chain of the T-cell receptor, CD3ε, CD3ζ, CD4, CD5, CD8α, CD9, CD16, CD22, CD27, CD28, CD33, CD37, CD45, CD64, CD80, CD86, CD134, CD137, CD152, CD154, and PD-1. In a particular embodiment, the TM domain is synthetic and predominantly comprises hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s).

In some aspects, the transmembrane domain contains a transmembrane portion of CD28. Exemplary sequences of transmembrane domains are or comprise the sequences set forth in SEQ ID NOs:46, 113, 119, 121, 175, or 176. In some embodiments, the transmembrane domain is or comprises the sequence set forth in SEQ ID NO:273.

In one embodiment, the CARs contemplated herein comprise a TM domain derived from CD8α. In certain embodiments In certain embodiments, a CAR contemplated herein comprises a TM domain derived from CD8α and a short oligo- or polypeptide linker, preferably between 1, 2, 3, 4, 5, 6, 7, 8, 9, or 10 amino acids in length that links the TM domain and the intracellular signaling domain of the CAR. Among the intracellular signaling domains are those that mimic or approximate a signal through a natural antigen receptor, a signal through such a receptor in combination with a costimulatory receptor, and/or a signal through a costimulatory receptor alone. A glycine-serine based linker provides a particularly suitable linker. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the CAR.

### 5. Intracellular Signaling Domain

In particular embodiments, CARs contemplated herein comprise an intracellular signaling domain. An “intracellular signaling domain” refers to the part of a CAR that participates in transducing the message of effective BCMA CAR binding to a human BCMA polypeptide into the interior of the immune effector cell to elicit effector cell function, e.g., activation, cytokine production, proliferation and cytotoxic activity, including the release of cytotoxic factors to the CAR-bound target cell, or other cellular responses elicited with antigen binding to the extracellular CAR domain.

The term “effector function” refers to a specialized function of an immune effector cell. Effector function of the T cell, for example, may be cytolytic activity or helper activity including the secretion of a cytokine. Thus, the term “intracellular signaling domain” refers to the portion of a protein which transduces the effector function signal and that directs the cell to perform a specialized function. While usually the entire intracellular signaling domain can be employed, in many cases it is not necessary to use the entire domain. To the extent that a truncated portion of an intracellular signaling domain is used, such truncated portion may be used in place of the entire domain as long as it transduces the effector function signal. The term intracellular signaling domain is meant to include any truncated portion of the intracellular signaling domain sufficient to transducing effector function signal.

In some embodiments, the receptor includes an intracellular component of a TCR complex, such as a TCR CD3 chain that mediates T-cell stimulation and/or activation and cytotoxicity, e.g., CD3 zeta chain. Thus, in some aspects, the antigen-binding portion is linked to one or more cell signaling modules. In some embodiments, cell signaling modules include CD3 transmembrane domain, CD3 intracellular signaling domains, and/or other CD transmembrane domains.

In some embodiments, upon ligation of the CAR or other chimeric receptor, the cytoplasmic domain or intracellular signaling domain of the receptor stimulates and/or activates at least one of the normal effector functions or responses of the immune cell, e.g., T cell engineered to express the CAR. For example, in some contexts, the CAR induces a function of a T cell such as cytolytic activity or T-helper activity, such as secretion of cytokines or other factors. In some embodiments, a truncated portion of an intracellular signaling domain of an antigen receptor component or costimulatory molecule is used in place of an intact immunostimulatory chain, for example, if it transduces the effector function signal. In some embodiments, the intracellular signaling domain or domains include the cytoplasmic sequences of the T cell receptor (TCR), and in some aspects also those of co-receptors that in the natural context act in concert with such receptors to initiate signal transduction following antigen receptor engagement, and/or any derivative or variant of such molecules, and/or any synthetic sequence that has the same functional capability.

It is known that signals generated through the TCR alone are insufficient for full activation of the T cell and that a secondary or co-stimulatory signal is also required. Thus, T cell activation can be said to be mediated by two distinct classes of intracellular signaling domains: primary signaling domains that initiate antigen-dependent primary activation through the TCR (e.g., a TCR/CD3 complex) and co-stimulatory signaling domains that act in an antigen-independent manner to provide a secondary or co-stimulatory signal. Thus, in some embodiments, to promote full activation, a component for generating secondary or co-stimulatory signal is also included in the CAR. In other embodiments, the CAR does not include a component for generating a costimulatory signal. In some aspects, an additional CAR is expressed in the same cell and provides the component for generating the secondary or costimulatory signal.

In certain embodiments, a CAR contemplated herein comprises an intracellular signaling domain that comprises one or more “co-stimulatory signaling domain” and a “primary signaling domain.”

Primary signaling domains regulate primary activation of the TCR complex either in a stimulatory way, or in an inhibitory way. Primary signaling domains that act in a stimulatory manner may contain signaling motifs which are known as immunoreceptor tyrosine-based activation motifs or ITAMs.

Illustrative examples of ITAM containing primary signaling domains that are of particular use in the subject matter presented herein include those derived from TCRζ, FcRγ, FcRβ, CD3γ, CD3δ, CD3ε, CD3ζ, CD22, CD79a, CD79b, and CD66d. In particular embodiments, a CAR comprises a CD3ζ primary signaling domain and one or more co-stimulatory signaling domains. In some embodiments, the receptor, e.g., CAR, further includes a portion of one or more additional molecules such as Fc receptor γ, CD8, CD4, CD25 or CD16. For example, in some aspects, the CAR or other chimeric receptor includes a chimeric molecule between CD3-zeta (CD3-ζ) or Fc receptor γ and CD8, CD4, CD25 or CD16. The intracellular primary signaling and co-stimulatory signaling domains may be linked in any order in tandem to the carboxyl terminus of the transmembrane domain.

CARs contemplated herein comprise one or more co-stimulatory signaling domains to enhance the efficacy and expansion of T cells expressing CAR receptors. As used herein, the term, “co-stimulatory signaling domain,” or “co-stimulatory domain,” refers to an intracellular signaling domain of a co-stimulatory molecule. Co-stimulatory molecules are cell surface molecules other than antigen receptors or Fc receptors that provide a second signal required for efficient activation and function of T lymphocytes upon binding to antigen. Illustrative examples of such co-stimulatory molecules include CARD11, CD2, CD7, CD27, CD28, CD30, CD40, CD54 (ICAM), CD83, CD134 (OX40), CD137 (4-1BB), CD150 (SLAMF1), CD152 (CTLA4), CD223 (LAG3), CD270 (HVEM), CD273 (PD-L2), CD274 (PD-L1), CD278 (ICOS), DAP10, LAT, NKD2C SLP76, TRIM, and ZAP70. In one embodiment, a CAR comprises one or more co-stimulatory signaling domains selected from the group consisting of CD28, CD137, and CD134, and a CD3ζ primary signaling domain.

In certain embodiments, a CAR comprises CD28 and CD137 co-stimulatory signaling domains and a CD3ζ primary signaling domain.

In yet another embodiment, a CAR comprises CD28 and CD134 co-stimulatory signaling domains and a CD3ζ primary signaling domain.

In one embodiment, a CAR comprises CD137 and CD134 co-stimulatory signaling domains and a CD3ζ primary signaling domain.

In some embodiments, the CAR includes a signaling region and/or transmembrane portion of a costimulatory receptor, such as CD28, 4-1BB, OX40 (CD134), CD27, DAP10, DAP12, ICOS and/or other costimulatory receptors. In some aspects, the same CAR includes both the primary cytoplasmic signaling region and costimulatory signaling components. In some embodiments, the chimeric antigen receptor contains an intracellular domain derived from a T cell costimulatory molecule or a functional variant thereof, such as between the transmembrane domain and intracellular signaling domain. In some aspects, the T cell costimulatory molecule is CD28 or 41BB.

In some embodiments, one or more different recombinant receptors can contain one or more different intracellular signaling region(s) or domain(s). In some embodiments, the primary cytoplasmic signaling region is included within one CAR, whereas the costimulatory component is provided by another receptor, e.g., another CAR recognizing another antigen. In some embodiments, the CARs include activating or stimulatory CARs, and costimulatory CARs, both expressed on the same cell (see WO2014/055668).

In some aspects, the cells include one or more stimulatory or activating CAR and/or a costimulatory CAR. In some embodiments, the cells further include inhibitory CARs (iCARs, see Fedorov et al., Sci. Transl. Medicine, 5(215) (2013), such as a CAR recognizing an antigen other than the one associated with and/or specific for the disease or condition whereby an activating signal delivered through the disease-targeting CAR is diminished or inhibited by binding of the inhibitory CAR to its ligand, e.g., to reduce off-target effects.

In some embodiments, the two receptors induce, respectively, an activating and an inhibitory signal to the cell, such that ligation of one of the receptor to its antigen activates the cell or induces a response, but ligation of the second inhibitory receptor to its antigen induces a signal that suppresses or dampens that response. Examples are combinations of activating CARs and inhibitory CARs (iCARs). Such a strategy may be used, for example, to reduce the likelihood of off-target effects in the context in which the activating CAR binds an antigen expressed in a disease or condition but which is also expressed on normal cells, and the inhibitory receptor binds to a separate antigen which is expressed on the normal cells but not cells of the disease or condition.

In some aspects, the chimeric receptor is or includes an inhibitory CAR (e.g. iCAR) and includes intracellular components that dampen or suppress an immune response, such as an ITAM- and/or co stimulatory-promoted response in the cell. Exemplary of such intracellular signaling components are those found on immune checkpoint molecules, including PD-1, CTLA4, LAG3, BTLA, OX2R, TIM-3, TIGIT, LAIR-1, PGE2 receptors, EP2/4 Adenosine receptors including A2AR. In some aspects, the engineered cell includes an inhibitory CAR including a signaling domain of or derived from such an inhibitory molecule, such that it serves to dampen the response of the cell, for example, that induced by an activating and/or costimulatory CAR.

In certain embodiments, the intracellular signaling domain comprises a CD28 transmembrane and signaling domain linked to a CD3 (e.g., CD3-zeta) intracellular domain. In some embodiments, the intracellular signaling domain comprises a chimeric CD28 and CD137 (4-1BB, TNFRSF9) co-stimulatory domains, linked to a CD3 zeta intracellular domain.

In some embodiments, the CAR encompasses one or more, e.g., two or more, costimulatory domains and primary cytoplasmic signaling region, in the cytoplasmic portion. Exemplary CARs include intracellular components, such as intracellular signaling region(s) or domain(s), of CD3-zeta, CD28, CD137 (4-1BB), OX40 (CD134), CD27, DAP10, DAP12, NKG2D and/or ICOS. In some embodiments, the chimeric antigen receptor contains an intracellular signaling region or domain of a T cell costimulatory molecule, e.g., from CD28, CD137 (4-1BB), OX40 (CD134), CD27, DAP10, DAP12, NKG2D and/or ICOS, in some cases, between the transmembrane domain and intracellular signaling region or domain. In some aspects, the T cell costimulatory molecule is one or more of CD28, CD137 (4-1BB), OX40 (CD134), CD27, DAP10, DAP12, NKG2D and/or ICOS.

In some cases, CARs are referred to as first, second, and/or third generation CARs. In some aspects, a first generation CAR is one that solely provides a CD3-chain induced signal upon antigen binding; in some aspects, a second-generation CARs is one that provides such a signal and costimulatory signal, such as one including an intracellular signaling domain from a costimulatory receptor such as CD28 or CD137; in some aspects, a third generation CAR is one that includes multiple costimulatory domains of different costimulatory receptors.

In some embodiments, the chimeric antigen receptor includes an extracellular portion containing an antibody or antibody fragment. In some aspects, the chimeric antigen receptor includes an extracellular portion containing the antibody or fragment and an intracellular signaling domain. In some embodiments, the antibody or fragment includes an scFv and the intracellular domain contains an ITAM. In some aspects, the intracellular signaling domain includes a signaling domain of a zeta chain of a CD3-zeta (CD3ζ) chain. In some embodiments, the chimeric antigen receptor includes a transmembrane domain linking the extracellular domain and the intracellular signaling domain. In some aspects, the transmembrane domain contains a transmembrane portion of CD28. In some embodiments, the chimeric antigen receptor contains an intracellular domain of a T cell costimulatory molecule. The extracellular domain and transmembrane domain can be linked directly or indirectly. In some embodiments, the extracellular domain and transmembrane are linked by a spacer, such as any described herein. In some embodiments, the receptor contains extracellular portion of the molecule from which the transmembrane domain is derived, such as a CD28 extracellular portion. In some embodiments, the chimeric antigen receptor contains an intracellular domain derived from a T cell costimulatory molecule or a functional variant thereof, such as between the transmembrane domain and intracellular signaling domain. In some aspects, the T cell costimulatory molecule is CD28 or 41BB.

In some embodiments, the CAR contains an antibody, e.g., an antibody fragment, a transmembrane domain that is or contains a transmembrane portion of CD28 or a functional variant thereof, and an intracellular signaling domain containing a signaling portion of CD28 or functional variant thereof and a signaling portion of CD3 zeta or functional variant thereof. In some embodiments, the CAR contains an antibody, e.g., antibody fragment, a transmembrane domain that is or contains a transmembrane portion of CD28 or a functional variant thereof, and an intracellular signaling domain containing a signaling portion of a 4-1BB or functional variant thereof and a signaling portion of CD3 zeta or functional variant thereof. In some such embodiments, the receptor further includes a spacer containing a portion of an Ig molecule, such as a human Ig molecule, such as an Ig hinge, e.g. an IgG4 hinge, such as a hinge-only spacer.

In some embodiments, the transmembrane domain of the recombinant receptor, e.g., the CAR, is or includes a transmembrane domain of human CD28 (e.g. Accession No. P10747.1), or CD8a (Accession No. P01732.1), or variant thereof, such as a transmembrane domain that comprises the sequence of amino acids set forth in SEQ ID NOs:46, 113, 175, or 176 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NOs:46, 113, 175, or 176. In some embodiments, the transmembrane-domain containing portion of the recombinant receptor comprises the sequence of amino acids set forth in SEQ ID NO:47 or a sequence of amino acids having at least at or about 85%, 86%, 87%, 88%, 89%, 90%, 91%, 92%, 93%, 94%, 95%, 96%, 97%, 98%, 99% or more sequence identity thereto.

In some embodiments, the transmembrane domain is a transmembrane domain from CD8α. In some embodiments, the transmembrane domain is any as described in Milone et al., Mol. Ther. (2009) 12(9):1453-64. In some embodiments, the transmembrane domain is or comprises the sequence set forth in SEQ ID NO: 176.

In some embodiments, the intracellular signaling component(s) of the recombinant receptor, e.g. the CAR, contains an intracellular costimulatory signaling domain of human CD28 or a functional variant or portion thereof, such as a domain with an LL to GG substitution at positions 186-187 of a native CD28 protein. For example, the intracellular signaling domain can comprise the sequence of amino acids set forth in SEQ ID NOs:48 or 49 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NOs:48 or 49. In some embodiments, the intracellular domain comprises an intracellular costimulatory signaling domain of 4-1BB (e.g. Accession No. Q07011.1) or functional variant or portion thereof, such as the sequence of amino acids set forth in SEQ ID NO:50 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO:50.

In some embodiments, the intracellular domain comprises an intracellular costimulatory signaling domain of 4-1BB. In some embodiments, the 4-1BB co-stimulatory molecule is any as described in Milone et al., Mol. Ther. (2009) 12(9):1453-64. In some embodiments, the co-stimulatory molecular has the sequence set forth in SEQ ID NO:50.

In some embodiments, the intracellular signaling domain of the recombinant receptor, e.g. the CAR, comprises a human CD3 zeta stimulatory signaling domain or functional variant thereof, such as a 112 AA cytoplasmic domain of isoform 3 of human CD3ζ (Accession No. P20963.2) or a CD3 zeta signaling domain as described in U.S. Pat. No. 7,446,190 or U.S. Pat. No. 8,911,993. For example, in some embodiments, the intracellular signaling domain comprises the sequence of amino acids as set forth in SEQ ID NOs:51, 52, or 53, or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NOs:51, 52, or 53. In some embodiments, the CD3-zeta domain is any as described in Milone et al., Mol. Ther. (2009) 12(9):1453-64. In some embodiments, the CD3-zeta is or comprises the sequence set forth in SEQ ID NO:51.

In some aspects, the spacer contains only a hinge region of an IgG, such as only a hinge of IgG4 or IgG1, such as the hinge only spacer set forth in SEQ ID NO:39 or SEQ ID NO: 123. In other embodiments, the spacer is or contains an Ig hinge, e.g., an IgG4-derived hinge, optionally linked to a C_{H}2 and/or C_{H}3 domains. In some embodiments, the spacer is an Ig hinge, e.g., an IgG4 hinge, linked to C_{H}2 and C_{H}3 domains, such as set forth in SEQ ID NO:42. In some embodiments, the spacer is an Ig hinge, e.g., an IgG4 hinge, linked to a C_{H}3 domain only, such as set forth in SEQ ID NO:41. In some embodiments, the spacer is or comprises a glycine-serine rich sequence or other flexible linker such as known flexible linkers. In some embodiments, the spacer is a CD8a hinge, such as set forth in any of SEQ ID NOs:115-117, an FcγRIIIa hinge, such as set forth in SEQ ID NO: 122, a CTLA4 hinge, such as set forth in SEQ ID NO: 118, or a PD-1 hinge, such as set forth in SEQ ID NO: 120. In some embodiments the spacer is derived from CD8. In some embodiments, the spacer is a CD8α hinge sequence. In some embodiments, the hinge sequence is any as described in Milone et al., Mol. Ther. (2009) 12(9):1453-64. In some embodiments, the hinge is or comprises the sequence set forth in SEQ ID NO: 116.

For example, in some embodiments, the CAR includes an antibody such as an antibody fragment, including scFvs, a spacer, such as a spacer containing a portion of an immunoglobulin molecule, such as a hinge region and/or one or more constant regions of a heavy chain molecule, such as an Ig-hinge containing spacer, a transmembrane domain containing all or a portion of a CD28-derived transmembrane domain, a CD28-derived intracellular signaling domain, and a CD3 zeta signaling domain. In some embodiments, the CAR includes an antibody or fragment, such as scFv, a spacer such as any of the Ig-hinge containing spacers, a CD28-derived transmembrane domain, a 4-1BB-derived intracellular signaling domain, and a CD3 zeta-derived signaling domain. In some embodiments, the CAR includes an antibody or fragment, such as scFv, a spacer such as any of the Ig-hinge containing spacers, a CD8-derived transmembrane domain, a 4-1BB-derived intracellular signaling domain, and a CD3 zeta-derived signaling domain.

In particular embodiments, CARs contemplated herein comprise a human anti-BCMA antibody or antigen binding fragment thereof that specifically binds to a BCMA polypeptide expressed on B cells, e.g., a human BCMA expressed on human B cells.

In particular embodiments, CARs contemplated herein comprise a murine anti-BCMA antibody or antigen binding fragment thereof that specifically binds to a BCMA polypeptide expressed on B cells, e.g., a human BCMA expressed on human B cells.

In one embodiment, a CAR comprises a murine anti-BCMA scFv that binds a BCMA polypeptide, e.g., a human BCMA polypeptide; a transmembrane domain derived from a polypeptide selected from the group consisting of: alpha, beta or zeta chain of the T-cell receptor, CD3ε, CD3ζ, CD4, CD5, CD8α, CD9, CD 16, CD22, CD27, CD28, CD33, CD37, CD45, CD64, CD80, CD86, CD 134, CD137, CD152, CD 154, and PD1; and one or more intracellular co-stimulatory signaling domains from a co-stimulatory molecule selected from the group consisting of: CARD11, CD2, CD7, CD27, CD28, CD30, CD40, CD54 (ICAM), CD83, CD134 (OX40), CD137 (4-1BB), CD150 (SLAMF1), CD152 (CTLA4), CD223 (LAG3), CD270 (HVEM), CD273 (PD-L2), CD274 (PD-L1), CD278 (ICOS), DAP10, LAT, NKD2C SLP76, TRIM, and ZAP70; and a primary signaling domain from TCRζ, FcRγ, FcRβ, CD3γ, CD3δ, CD3ε, CD3ζ, CD22, CD79a, CD79b, and CD66d.

In one embodiment, a CAR comprises a murine anti-BCMA scFv that binds a BCMA polypeptide, e.g., a human BCMA polypeptide; a transmembrane domain derived from a polypeptide selected from the group consisting of: alpha, beta or zeta chain of the T-cell receptor, CD3ε, CD3ζ, CD4, CD5, CD8α, CD9, CD 16, CD22, CD27, CD28, CD33, CD37, CD45, CD64, CD80, CD86, CD 134, CD137, CD152, CD 154, and PD1; and one or more intracellular co-stimulatory signaling domains from a co-stimulatory molecule selected from the group consisting of: CARD11, CD2, CD7, CD27, CD28, CD30, CD40, CD54 (ICAM), CD83, CD134 (OX40), CD137 (4-1BB), CD150 (SLAMF1), CD152 (CTLA4), CD223 (LAG3), CD270 (HVEM), CD273 (PD-L2), CD274 (PD-L1), CD278 (ICOS), DAP10, LAT, NKD2C SLP76, TRIM, and ZAP70; and one or more primary signaling domains from a polypeptide selected from the group consisting of: TCRζ, FcRγ, FcRβ, CD3γ, CD3δ, CD3ε, CD3ζ, CD22, CD79a, CD79b, and CD66d.

In one embodiment, a CAR comprises a murine anti-BCMA scFv that binds a BCMA polypeptide, e.g., a human BCMA polypeptide, a hinge domain selected from the group consisting of: IgG1 hinge/CH2/CH3, IgG4 hinge/CH2/CH3, and a CD8α hinge; a transmembrane domain derived from a polypeptide selected from the group consisting of: alpha, beta or zeta chain of the T-cell receptor, CD3ε, CD3ζ, CD4, CD5, CD8α, CD9, CD 16, CD22, CD27, CD28, CD33, CD37, CD45, CD64, CD80, CD86, CD 134, CD137, CD152, CD 154, and PD1; and one or more intracellular co-stimulatory signaling domains from a co-stimulatory molecule selected from the group consisting of: CARD11, CD2, CD7, CD27, CD28, CD30, CD40, CD54 (ICAM), CD83, CD134 (OX40), CD137 (4-1BB), CD150 (SLAMF1), CD152 (CTLA4), CD223 (LAG3), CD270 (HVEM), CD273 (PD-L2), CD274 (PD-L1), CD278 (ICOS), DAP10, LAT, NKD2C SLP76, TRIM, and ZAP70; and a primary signaling domain from TCRζ, FcRγ, FcRβ, CD3γ, CD3δ, CD3ε, CD3ζ, CD22, CD79a, CD79b, and CD66d.

In one embodiment, a CAR comprises a murine anti-BCMA scFv that binds a BCMA polypeptide, e.g., a human BCMA polypeptide; a hinge domain selected from the group consisting of: IgG1 hinge/CH2/CH3, IgG4 hinge/CH2/CH3, and a CD8α hinge; a transmembrane domain derived from a polypeptide selected from the group consisting of: alpha, beta or zeta chain of the T-cell receptor, CD3ε, CD3ζ, CD4, CD5, CD8α, CD9, CD 16, CD22, CD27, CD28, CD33, CD37, CD45, CD64, CD80, CD86, CD 134, CD137, CD152, CD 154, and PD1; and one or more intracellular co-stimulatory signaling domains from a co-stimulatory molecule selected from the group consisting of: CARD11, CD2, CD7, CD27, CD28, CD30, CD40, CD54 (ICAM), CD83, CD134 (OX40), CD137 (4-1BB), CD150 (SLAMF1), CD152 (CTLA4), CD223 (LAG3), CD270 (HVEM), CD273 (PD-L2), CD274 (PD-L1), CD278 (ICOS), DAP10, LAT, NKD2C SLP76, TRIM, and ZAP70; and one or more primary signaling domains from a polypeptide selected from the group consisting of: TCRζ, FcRγ, FcRβ, CD3γ, CD3δ, CD3ε, CD3ζ, CD22, CD79a, CD79b, and CD66d.

In one embodiment, a CAR comprises a murine anti-BCMA scFv that binds a BCMA polypeptide, e.g., a human BCMA polypeptide; a hinge domain selected from the group consisting of: IgG1 hinge/CH2/CH3, IgG4 hinge/CH2/CH3, and a CD8α hinge; a transmembrane domain derived from a polypeptide selected from the group consisting of: alpha, beta or zeta chain of the T-cell receptor, CD3ε, CD3ζ, CD4, CD5, CD8α, CD9, CD 16, CD22, CD27, CD28, CD33, CD37, CD45, CD64, CD80, CD86, CD 134, CD137, CD152, CD 154, and PD1; a short oligo- or polypeptide linker, preferably between 1, 2, 3, 4, 5, 6, 7, 8, 9, or 10 amino acids in length that links the TM domain to the intracellular signaling domain of the CAR; and one or more intracellular co-stimulatory signaling domains from a co-stimulatory molecule selected from the group consisting of: CARD11, CD2, CD7, CD27, CD28, CD30, CD40, CD54 (ICAM), CD83, CD134 (OX40), CD137 (4-1BB), CD150 (SLAMF1), CD152 (CTLA4), CD223 (LAG3), CD270 (HVEM), CD273 (PD-L2), CD274 (PD-L1), CD278 (ICOS), DAP10, LAT, NKD2C SLP76, TRIM, and ZAP70; and a primary signaling domain from TCRζ, FcRγ, FcRβ, CD3γ, CD3δ, CD3ε, CD3ζ, CD22, CD79a, CD79b, and CD66d.

In one embodiment, a CAR comprises a murine anti-BCMA scFv that binds a BCMA polypeptide, e.g., a human BCMA polypeptide; a hinge domain selected from the group consisting of: IgG1 hinge/CH2/CH3, IgG4 hinge/CH2/CH3, and a CD8α hinge; a transmembrane domain derived from a polypeptide selected from the group consisting of: alpha, beta or zeta chain of the T-cell receptor, CD3ε, CD3ζ, CD4, CD5, CD8α, CD9, CD 16, CD22, CD27, CD28, CD33, CD37, CD45, CD64, CD80, CD86, CD 134, CD137, CD152, CD 154, and PD1; a short oligo- or polypeptide linker, preferably between 1, 2, 3, 4, 5, 6, 7, 8, 9, or 10 amino acids in length that links the TM domain to the intracellular signaling domain of the CAR; and one or more intracellular co-stimulatory signaling domains from a co-stimulatory molecule selected from the group consisting of: CARD11, CD2, CD7, CD27, CD28, CD30, CD40, CD54 (ICAM), CD83, CD134 (OX40), CD137 (4-1BB), CD150 (SLAMF1), CD152 (CTLA4), CD223 (LAG3), CD270 (HVEM), CD273 (PD-L2), CD274 (PD-L1), CD278 (ICOS), DAP10, LAT, NKD2C SLP76, TRIM, and ZAP70; and one or more primary signaling domains from a polypeptide selected from the group consisting of: TCRζ, FcRγ, FcRβ, CD3γ, CD3δ, CD3ε, CD3ζ, CD22, CD79a, CD79b, and CD66d.

In a particular embodiment, a CAR comprises a murine anti-BCMA scFv that binds a BCMA polypeptide, e.g., a human BCMA polypeptide; a hinge domain comprising an IgG1 hinge/CH2/CH3 polypeptide and a CD8α polypeptide; a CD8α transmembrane domain comprising a polypeptide linker of about 3 to about 10 amino acids; a CD137 intracellular co-stimulatory signaling domain; and a CD3ζ primary signaling domain.

In a particular embodiment, a CAR comprises a murine anti-BCMA scFv that binds a BCMA polypeptide, e.g., a human BCMA polypeptide; a hinge domain comprising a CD8α polypeptide; a CD8α transmembrane domain comprising a polypeptide linker of about 3 to about 10 amino acids; a CD134 intracellular co-stimulatory signaling domain; and a CD3ζ primary signaling domain.

In a particular embodiment, a CAR comprises a murine anti-BCMA scFv that binds a BCMA polypeptide, e.g., a human BCMA polypeptide; a hinge domain comprising a CD8α polypeptide; a CD8α transmembrane domain comprising a polypeptide linker of about 3 to about 10 amino acids; a CD28 intracellular co-stimulatory signaling domain; and a CD3ζ primary signaling domain.

In a particular embodiment, a CAR comprises a murine anti-BCMA scFv that binds a BCMA polypeptide, e.g., a human BCMA polypeptide; a hinge domain comprising a CD8α polypeptide; a CD8α transmembrane domain; a CD137 (4-1BB) intracellular co-stimulatory signaling domain; and a CD3ζ primary signaling domain.

Moreover, the design of the CARs contemplated herein enable improved expansion, long-term persistence, and tolerable cytotoxic properties in T cells expressing the CARs compared to non-modified T cells or T cells modified to express other CARs.

### 6. Other

In some embodiments, the antigen receptor further includes a marker and/or cells expressing the CAR or other antigen receptor further includes a surrogate marker, such as a cell surface marker, which may be used to confirm transduction or engineering of the cell to express the receptor. In some embodiments, the marker is a molecule, e.g., cell surface protein, not naturally found on T cells or not naturally found on the surface of T cells, or a portion thereof. In some embodiments, the molecule is a non-self molecule, e.g., non-self protein, i.e., one that is not recognized as “self” by the immune system of the host into which the cells will be adoptively transferred. In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand. In some aspects, the marker includes all or part (e.g., truncated form) of CD34, a NGFR, or epidermal growth factor receptor, such as truncated version of such a cell surface receptor (e.g., tEGFR). In some embodiments, the nucleic acid encoding the marker is operably linked to a polynucleotide encoding for a linker sequence, such as a cleavable linker sequence, e.g., T2A. For example, a marker, and optionally a linker sequence, can be any as disclosed in published patent application No. WO2014031687. For example, the marker can be a truncated EGFR (tEGFR) that is, optionally, linked to a linker sequence, such as a T2A cleavable linker sequence. In some embodiments, such CAR constructs further includes a T2A ribosomal skip element and/or a tEGFR sequence, e.g., downstream of the CAR.

An exemplary polypeptide for a truncated EGFR (e.g. tEGFR) comprises the sequence of amino acids set forth in SEQ ID NOs:45 or 199 or a sequence of amino acids that exhibits at least 85%, 86%, 87%, 88%, 89%, 90%, 91%, 92%, 93% 94% 95%, 96%, 97% 98%, 99% or more sequence identity to SEQ ID NOs:45 or 199. An exemplary T2A linker sequence comprises the sequence of amino acids set forth in SEQ ID NOs:44 or 200 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NOs:44 or 200.

In some embodiments, nucleic acid molecules encoding such CAR constructs further includes a sequence encoding a T2A ribosomal skip element and/or a tEGFR sequence, e.g., downstream of the sequence encoding the CAR. In some embodiments, the sequence encodes a T2A ribosomal skip element set forth in SEQ ID NOs:44 or 200, or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NOs:44 or 200. In some embodiments, T cells expressing an antigen receptor (e.g. CAR) can also be generated to express a truncated EGFR (EGFRt) as a non-immunogenic selection epitope (e.g. by introduction of a construct encoding the CAR and EGFRt separated by a T2A ribosome switch to express two proteins from the same construct), which then can be used as a marker to detect such cells (see e.g. U.S. Pat. No. 8,802,374). In some embodiments, the sequence encodes an tEGFR sequence set forth in SEQ ID NOs:45 or 199, or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NOs:45 or 199.

An exemplary P2A linker sequence comprises the sequence of amino acids set forth in SEQ ID NOs:201 or 202 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NOs:201 or 202. An exemplary E2A linker sequence comprises the sequence of amino acids set forth in SEQ ID NO:203 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO:203. An exemplary F2A linker sequence comprises the sequence of amino acids set forth in SEQ ID NO:204 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO:204. In some embodiments, the sequence encodes a P2A ribosomal skip element set forth in SEQ ID NOs:201 or 202, or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NOs:201 or 202. In some embodiments, the sequence encodes a E2A ribosomal skip element set forth in SEQ ID NO:203, or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO:203. In some embodiments, the sequence encodes a F2A ribosomal skip element set forth in SEQ ID NO:204, or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO:204.

In some embodiments, the encoded CAR can sequence can further include a signal sequence or signal peptide that directs or delivers the CAR to the surface of the cell in which the CAR is expressed. In some embodiments, the signal peptide is derived from a transmembrane protein. In some examples the signal peptide is derived from CD8a, CD33, or an IgG. In some embodiments, the signal peptide comprise the sequence set forth in SEQ ID NO: 189 that is encoded by the polynucleotide sequence set forth in SEQ ID NO: 188. Exemplary signal peptides include the sequences set forth in SEQ ID NOs:73, 74, 186, and 187. In some examples the signal peptide is derived from CD8α. In some embodiments, the signal peptide is the sequence set forth in Accession No. NM_001768. In some embodiments, the signal peptide is the sequence set for in SEQ ID NO:73.

### B. Polynucleotides

In certain embodiments, a polynucleotide encoding one or more CAR polypeptides is provided, e.g., SEQ ID NO:10. As used herein, the terms “polynucleotide” or “nucleic acid” refers to messenger RNA (mRNA), RNA, genomic RNA (gRNA), plus strand RNA (RNA(+)), minus strand RNA (RNA(−)), genomic DNA (gDNA), complementary DNA (cDNA) or recombinant DNA. Polynucleotides include single and double stranded polynucleotides. Preferably, polynucleotides disclosed herein include polynucleotides or variants having at least about 50%, 55%, 60%, 65%, 70%, 75%, 80%, 85%, 90%, 95%, 96%, 97%, 98%, 99% or 100% sequence identity to any of the reference sequences described herein (see, e.g., Sequence Listing), typically where the variant maintains at least one biological activity of the reference sequence. In various illustrative embodiments, the present disclosure contemplates, in part, polynucleotides comprising expression vectors, viral vectors, and transfer plasmids, and compositions, and cells comprising the same.

In particular embodiments, polynucleotides are provided by this disclosure that encode at least about 5, 10, 25, 50, 100, 150, 200, 250, 300, 350, 400, 500, 1000, 1250, 1500, 1750, or 2000 or more contiguous amino acid residues of a polypeptide, as well as all intermediate lengths. It will be readily understood that “intermediate lengths,” in this context, means any length between the quoted values, such as 6, 7, 8, 9, etc.; 101, 102, 103, etc.; 151, 152, 153, etc.; 201, 202, 203, etc.

As used herein, the terms “polynucleotide variant” and “variant” and the like refer to polynucleotides displaying substantial sequence identity with a reference polynucleotide sequence or polynucleotides that hybridize with a reference sequence under stringent conditions that are defined hereinafter. These terms include polynucleotides in which one or more nucleotides have been added or deleted, or replaced with different nucleotides compared to a reference polynucleotide. In this regard, it is well understood in the art that certain alterations inclusive of mutations, additions, deletions and substitutions can be made to a reference polynucleotide whereby the altered polynucleotide retains the biological function or activity of the reference polynucleotide.

The recitations “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. Included are nucleotides and polypeptides having at least about 50%, 55%, 60%, 65%, 70%, 75%, 80%, 85%, 90%, 95%, 96%, 97%, 98%, 99% or 100% sequence identity to any of the reference sequences described herein, typically where the polypeptide variant maintains at least one biological activity of the reference polypeptide.

Terms used to describe sequence relationships between two or more polynucleotides or polypeptides include “reference sequence,” “comparison window,” “sequence identity,” “percentage of sequence identity,” and “substantial identity”. A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, WI, USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., Current Protocols in Molecular Biology, John Wiley & Sons Inc, 1994-1998, Chapter 15.

As used herein, “isolated polynucleotide” refers to a polynucleotide that has been purified from the sequences which flank it in a naturally-occurring state, e.g., a DNA fragment that has been removed from the sequences that are normally adjacent to the fragment. An “isolated polynucleotide” also refers to a complementary DNA (cDNA), a recombinant DNA, or other polynucleotide that does not exist in nature and that has been made by the hand of man.

Terms that describe the orientation of polynucleotides include: 5′ (normally the end of the polynucleotide having a free phosphate group) and 3′ (normally the end of the polynucleotide having a free hydroxyl (OH) group). Polynucleotide sequences can be annotated in the 5′ to 3′ orientation or the 3′ to 5′ orientation. For DNA and mRNA, the 5′ to 3′ strand is designated the “sense,” “plus,” or “coding” strand because its sequence is identical to the sequence of the premessenger (premRNA) [except for uracil (U) in RNA, instead of thymine (T) in DNA]. For DNA and mRNA, the complementary 3′ to 5′ strand which is the strand transcribed by the RNA polymerase is designated as “template,” “antisense,” “minus,” or “non-coding” strand. As used herein, the term “reverse orientation” refers to a 5′ to 3′ sequence written in the 3′ to 5′ orientation or a 3′ to 5′ sequence written in the 5′ to 3′ orientation.

The terms “complementary” and “complementarity” refer to polynucleotides (i.e., a sequence of nucleotides) related by the base-pairing rules. For example, the complementary strand of the DNA sequence 5′ A G T C A T G 3′ is 3′ T C A G T A C 5′. The latter sequence is often written as the reverse complement with the 5′ end on the left and the 3′ end on the right, 5′ C A T G A C T 3′. A sequence that is equal to its reverse complement is said to be a palindromic sequence. Complementarity can be “partial,” in which only some of the nucleic acids' bases are matched according to the base pairing rules. Or, there can be “complete” or “total” complementarity between the nucleic acids.

Moreover, it will be appreciated by those of ordinary skill in the art that, as a result of the degeneracy of the genetic code, there are many nucleotide sequences that encode a polypeptide, or fragment of variant thereof, as described herein. Some of these polynucleotides bear minimal homology to the nucleotide sequence of any native gene. Nonetheless, polynucleotides that vary due to differences in codon usage are specifically contemplated by the present disclosure, for example polynucleotides that are optimized for human and/or primate codon selection. Further, alleles of the genes comprising the polynucleotide sequences provided herein may also be used. Alleles are endogenous genes that are altered as a result of one or more mutations, such as deletions, additions and/or substitutions of nucleotides.

The term “nucleic acid cassette” as used herein refers to genetic sequences within a vector which can express a RNA, and subsequently a protein. The nucleic acid cassette contains the gene of interest, e.g., a CAR. The nucleic acid cassette is positionally and sequentially oriented within the vector such that the nucleic acid in the cassette can be transcribed into RNA, and when necessary, translated into a protein or a polypeptide, undergo appropriate post-translational modifications required for activity in the transformed cell, and be translocated to the appropriate compartment for biological activity by targeting to appropriate intracellular compartments or secretion into extracellular compartments. Preferably, the cassette has its 3′ and 5′ ends adapted for ready insertion into a vector, e.g., it has restriction endonuclease sites at each end. In one embodiment, the nucleic acid cassette contains the sequence of a chimeric antigen receptor used to treat a tumor or a cancer. In one embodiment, the nucleic acid cassette contains the sequence of a chimeric antigen receptor used to treat a B cell malignancy. The cassette can be removed and inserted into a plasmid or viral vector as a single unit.

In particular embodiments, polynucleotides include at least one polynucleotide-of-interest. As used herein, the term “polynucleotide-of-interest” refers to a polynucleotide encoding a polypeptide (i.e., a polypeptide-of-interest), inserted into an expression vector that is desired to be expressed. A vector may comprise 1, 2, 3, 4, 5, 6, 7, 8, 9, or 10 polynucleotides-of-interest. In certain embodiments, the polynucleotide-of-interest encodes a polypeptide that provides a therapeutic effect in the treatment or prevention of a disease or disorder. Polynucleotides-of-interest, and polypeptides encoded therefrom, include both polynucleotides that encode wild-type polypeptides, as well as functional variants and fragments thereof. In particular embodiments, a functional variant has at least 80%, at least 90%, at least 95%, or at least 99% identity to a corresponding wild-type reference polynucleotide or polypeptide sequence. In certain embodiments, a functional variant or fragment has at least 50%, at least 60%, at least 70%, at least 80%, or at least 90% of a biological activity of a corresponding wild-type polypeptide.

In one embodiment, the polynucleotide-of-interest does not encode a polypeptide but serves as a template to transcribe miRNA, siRNA, or shRNA, ribozyme, or other inhibitory RNA. In various other embodiments, a polynucleotide comprises a polynucleotide-of-interest encoding a CAR and one or more additional polynucleotides-of-interest including but not limited to an inhibitory nucleic acid sequence including, but not limited to: an siRNA, an miRNA, an shRNA, and a ribozyme.

As used herein, the terms “siRNA” or “short interfering RNA” refer to a short polynucleotide sequence that mediates a process of sequence-specific post-transcriptional gene silencing, translational inhibition, transcriptional inhibition, or epigenetic RNAi in animals (Zamore et al., 2000, Cell, 101, 25-33; Fire et al., 1998, Nature, 391, 806; Hamilton et al., 1999, Science, 286, 950-951; Lin et al., 1999, Nature, 402, 128-129; Sharp, 1999, Genes & Dev., 13, 139-141; and Strauss, 1999, Science, 286, 886). In certain embodiments, an siRNA comprises a first strand and a second strand that have the same number of nucleosides; however, the first and second strands are offset such that the two terminal nucleosides on the first and second strands are not paired with a residue on the complimentary strand. In certain instances, the two nucleosides that are not paired are thymidine resides. The siRNA should include a region of sufficient homology to the target gene, and be of sufficient length in terms of nucleotides, such that the siRNA, or a fragment thereof, can mediate down regulation of the target gene. Thus, an siRNA includes a region which is at least partially complementary to the target RNA. It is not necessary that there be perfect complementarity between the siRNA and the target, but the correspondence must be sufficient to enable the siRNA, or a cleavage product thereof, to direct sequence specific silencing, such as by RNAi cleavage of the target RNA. Complementarity, or degree of homology with the target strand, is most critical in the antisense strand. While perfect complementarity, particularly in the antisense strand, is often desired, some embodiments include one or more, but preferably 10, 8, 6, 5, 4, 3, 2, or fewer mismatches with respect to the target RNA. The mismatches are most tolerated in the terminal regions, and if present are preferably in a terminal region or regions, e.g., within 6, 5, 4, or 3 nucleotides of the 5′ and/or 3′ terminus. The sense strand need only be sufficiently complementary with the antisense strand to maintain the overall double-strand character of the molecule.

In addition, an siRNA may be modified or include nucleoside analogs. Single stranded regions of an siRNA may be modified or include nucleoside analogs, e.g., the unpaired region or regions of a hairpin structure, e.g., a region which links two complementary regions, can have modifications or nucleoside analogs. Modification to stabilize one or more 3′- or 5′-terminus of an siRNA, e.g., against exonucleases, or to favor the antisense siRNA agent to enter into RISC are also useful. Modifications can include C3 (or C6, C7, C12) amino linkers, thiol linkers, carboxyl linkers, non-nucleotidic spacers (C3, C6, C9, C12, abasic, triethylene glycol, hexaethylene glycol), special biotin or fluorescein reagents that come as phosphoramidites and that have another DMT-protected hydroxyl group, allowing multiple couplings during RNA synthesis. Each strand of an siRNA can be equal to or less than 30, 25, 24, 23, 22, 21, or 20 nucleotides in length. The strand is preferably at least 19 nucleotides in length. For example, each strand can be between 21 and 25 nucleotides in length. Preferred siRNAs have a duplex region of 17, 18, 19, 29, 21, 22, 23, 24, or 25 nucleotide pairs, and one or more overhangs of 2-3 nucleotides, preferably one or two 3′ overhangs, of 2-3 nucleotides.

As used herein, the terms “miRNA” or “microRNA” refer to small non-coding RNAs of 20-22 nucleotides, typically excised from ˜70 nucleotide fold-back RNA precursor structures known as pre-miRNAs. miRNAs negatively regulate their targets in one of two ways depending on the degree of complementarity between the miRNA and the target. First, miRNAs that bind with perfect or nearly perfect complementarity to protein-coding mRNA sequences induce the RNA-mediated interference (RNAi) pathway. miRNAs that exert their regulatory effects by binding to imperfect complementary sites within the 3′ untranslated regions (UTRs) of their mRNA targets, repress target-gene expression post-transcriptionally, apparently at the level of translation, through a RISC complex that is similar to, or possibly identical with, the one that is used for the RNAi pathway. Consistent with translational control, miRNAs that use this mechanism reduce the protein levels of their target genes, but the mRNA levels of these genes are only minimally affected. miRNAs encompass both naturally occurring miRNAs as well as artificially designed miRNAs that can specifically target any mRNA sequence. For example, in one embodiment, the skilled artisan can design short hairpin RNA constructs expressed as human miRNA (e.g., miR-30 or miR-21) primary transcripts. This design adds a Drosha processing site to the hairpin construct and has been shown to greatly increase knockdown efficiency (Pusch et al., 2004). The hairpin stem consists of 22-nt of dsRNA (e.g., antisense has perfect complementarity to desired target) and a 15-19-nt loop from a human miR. Adding the miR loop and miR30 flanking sequences on either or both sides of the hairpin results in greater than 10-fold increase in Drosha and Dicer processing of the expressed hairpins when compared with conventional shRNA designs without microRNA. Increased Drosha and Dicer processing translates into greater siRNA/miRNA production and greater potency for expressed hairpins.

As used herein, the terms “shRNA” or “short hairpin RNA” refer to double-stranded structure that is formed by a single self-complementary RNA strand. shRNA constructs containing a nucleotide sequence identical to a portion, of either coding or non-coding sequence, of the target gene are preferred for inhibition. RNA sequences with insertions, deletions, and single point mutations relative to the target sequence have also been found to be effective for inhibition. Greater than 90% sequence identity, or even 100% sequence identity, between the inhibitory RNA and the portion of the target gene is preferred. In certain preferred embodiments, the length of the duplex-forming portion of an shRNA is at least 20, 21 or 22 nucleotides in length, e.g., corresponding in size to RNA products produced by Dicer-dependent cleavage. In certain embodiments, the shRNA construct is at least 25, 50, 100, 200, 300 or 400 bases in length. In certain embodiments, the shRNA construct is 400-800 bases in length. shRNA constructs are highly tolerant of variation in loop sequence and loop size.

As used herein, the term “ribozyme” refers to a catalytically active RNA molecule capable of site-specific cleavage of target mRNA. Several subtypes have been described, e.g., hammerhead and hairpin ribozymes. Ribozyme catalytic activity and stability can be improved by substituting deoxyribonucleotides for ribonucleotides at noncatalytic bases. While ribozymes that cleave mRNA at site-specific recognition sequences can be used to destroy particular mRNAs, the use of hammerhead ribozymes is preferred. Hammerhead ribozymes cleave mRNAs at locations dictated by flanking regions that form complementary base pairs with the target mRNA. The sole requirement is that the target mRNA has the following sequence of two bases: 5′-UG-3′. The construction and production of hammerhead ribozymes is well known in the art.

In certain embodiments, a method of delivery of a polynucleotide-of-interest that comprises an siRNA, an miRNA, an shRNA, or a ribozyme comprises one or more regulatory sequences, such as, for example, a strong constitutive pol III, e.g., human U6 snRNA promoter, the mouse U6 snRNA promoter, the human and mouse H1 RNA promoter and the human tRNA-val promoter, or a strong constitutive pol II promoter, as described elsewhere herein.

The polynucleotides disclosed herein, regardless of the length of the coding sequence itself, may be combined with other DNA sequences, such as promoters and/or enhancers, untranslated regions (UTRs), signal sequences, Kozak sequences, polyadenylation signals, additional restriction enzyme sites, multiple cloning sites, internal ribosomal entry sites (IRES), recombinase recognition sites (e.g., LoxP, FRT, and Att sites), termination codons, transcriptional termination signals, and polynucleotides encoding self-cleaving polypeptides, epitope tags, as disclosed elsewhere herein or as known in the art, such that their overall length may vary considerably. It is therefore contemplated that a polynucleotide fragment of almost any length may be employed, with the total length preferably being limited by the ease of preparation and use in the intended recombinant DNA protocol.

Polynucleotides can be prepared, manipulated and/or expressed using any of a variety of well-established techniques known and available in the art. In order to express a desired polypeptide, a nucleotide sequence encoding the polypeptide, can be inserted into appropriate vector. Examples of vectors are plasmid, autonomously replicating sequences, and transposable elements. Additional exemplary vectors include, without limitation, plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. Examples of categories of animal viruses useful as vectors include, without limitation, retrovirus (including lentivirus), adenovirus, adeno-associated virus, herpesvirus (e.g., herpes simplex virus), poxvirus, baculovirus, papillomavirus, and papovavirus (e.g., SV40). Examples of expression vectors are pClneo vectors (Promega) for expression in mammalian cells; pLenti4/V5-DEST™, pLenti6/V5-DEST™, and pLenti6.2/V5-GW/lacZ (Invitrogen) for lentivirus-mediated gene transfer and expression in mammalian cells. In particular embodiments, he coding sequences of the chimeric proteins disclosed herein can be ligated into such expression vectors for the expression of the chimeric protein in mammalian cells.

In one embodiment, a vector encoding a CAR contemplated herein comprises the polynucleotide sequence set forth in SEQ ID NO:36.

In particular embodiments, the vector is an episomal vector or a vector that is maintained extrachromosomally. As used herein, the term “episomal” refers to a vector that is able to replicate without integration into host's chromosomal DNA and without gradual loss from a dividing host cell also meaning that said vector replicates extrachromosomally or episomally. The vector is engineered to harbor the sequence coding for the origin of DNA replication or “ori” from a lymphotrophic herpes virus or a gamma herpesvirus, an adenovirus, SV40, a bovine papilloma virus, or a yeast, specifically a replication origin of a lymphotrophic herpes virus or a gamma herpesvirus corresponding to oriP of EBV. In a particular aspect, the lymphotrophic herpes virus may be Epstein Barr virus (EBV), Kaposi's sarcoma herpes virus (KSHV), Herpes virus saimiri (HS), or Marek's disease virus (MDV). Epstein Barr virus (EBV) and Kaposi's sarcoma herpes virus (KSHV) are also examples of a gamma herpesvirus. Typically, the host cell comprises the viral replication transactivator protein that activates the replication.

The “control elements” or “regulatory sequences” present in an expression vector are those non-translated regions of the vector—origin of replication, selection cassettes, promoters, enhancers, translation initiation signals (Shine Dalgarno sequence or Kozak sequence) introns, a polyadenylation sequence, 5′ and 3′ untranslated regions—which interact with host cellular proteins to carry out transcription and translation. Such elements may vary in their strength and specificity. Depending on the vector system and host utilized, any number of suitable transcription and translation elements, including ubiquitous promoters and inducible promoters may be used.

In particular embodiments, a vector for utilization herein include, but are not limited to expression vectors and viral vectors, will include exogenous, endogenous, or heterologous control sequences such as promoters and/or enhancers. An “endogenous” control sequence is one which is naturally linked with a given gene in the genome. An “exogenous” control sequence is one which is placed in juxtaposition to a gene by means of genetic manipulation (i.e., molecular biological techniques) such that transcription of that gene is directed by the linked enhancer/promoter. A “heterologous” control sequence is an exogenous sequence that is from a different species than the cell being genetically manipulated.

The term “promoter” as used herein refers to a recognition site of a polynucleotide (DNA or RNA) to which an RNA polymerase binds. An RNA polymerase initiates and transcribes polynucleotides operably linked to the promoter. In particular embodiments, promoters operative in mammalian cells comprise an AT-rich region located approximately 25 to 30 bases upstream from the site where transcription is initiated and/or another sequence found 70 to 80 bases upstream from the start of transcription, a CNCAAT region where N may be any nucleotide.

The term “enhancer” refers to a segment of DNA which contains sequences capable of providing enhanced transcription and in some instances can function independent of their orientation relative to another control sequence. An enhancer can function cooperatively or additively with promoters and/or other enhancer elements. The term “promoter/enhancer” refers to a segment of DNA which contains sequences capable of providing both promoter and enhancer functions.

The term “operably linked” refers to a juxtaposition wherein the components described are in a relationship permitting them to function in their intended manner. In one embodiment, the term refers to a functional linkage between a nucleic acid expression control sequence (such as a promoter, and/or enhancer) and a second polynucleotide sequence, e.g., a polynucleotide-of-interest, wherein the expression control sequence directs transcription of the nucleic acid corresponding to the second sequence.

As used herein, the term “constitutive expression control sequence” refers to a promoter, enhancer, or promoter/enhancer that continually or continuously allows for transcription of an operably linked sequence. A constitutive expression control sequence may be a “ubiquitous” promoter, enhancer, or promoter/enhancer that allows expression in a wide variety of cell and tissue types or a “cell specific,” “cell type specific,” “cell lineage specific,” or “tissue specific” promoter, enhancer, or promoter/enhancer that allows expression in a restricted variety of cell and tissue types, respectively.

Illustrative ubiquitous expression control sequences suitable for use in particular embodiments presented herein include, but are not limited to, a cytomegalovirus (CMV) immediate early promoter, a viral simian virus 40 (SV40) (e.g., early or late), a Moloney murine leukemia virus (MoMLV) LTR promoter, a Rous sarcoma virus (RSV) LTR, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), â-kinesin (â-KIN), the human ROSA 26 locus (Irions et al., Nature Biotechnology 25, 1477-1482 (2007)), a Ubiquitin C promoter (UBC), a phosphoglycerate kinase-1 (PGK) promoter, a cytomegalovirus enhancer/chicken â-actin (CAG) promoter, a â-actin promoter and a myeloproliferative sarcoma virus enhancer, negative control region deleted, dl587rev primer-binding site substituted (MND) promoter (Challita et al., J Virol. 69(2):748-55 (1995)).

In one embodiment, a vector of the present disclosure comprises a MND promoter.

In one embodiment, a vector of the present disclosure comprises an EF1a promoter comprising the first intron of the human EF1a gene.

In one embodiment, a vector of the present disclosure comprises an EF1a promoter that lacks the first intron of the human EF1a gene.

In a particular embodiment, it may be desirable to express a polynucleotide comprising a CAR from a T cell specific promoter.

As used herein, “conditional expression” may refer to any type of conditional expression including, but not limited to, inducible expression; repressible expression; expression in cells or tissues having a particular physiological, biological, or disease state, etc. This definition is not intended to exclude cell type or tissue specific expression. Certain embodiments provide conditional expression of a polynucleotide-of-interest, e.g., expression is controlled by subjecting a cell, tissue, organism, etc., to a treatment or condition that causes the polynucleotide to be expressed or that causes an increase or decrease in expression of the polynucleotide encoded by the polynucleotide-of-interest.

Illustrative examples of inducible promoters/systems include, but are not limited to, steroid-inducible promoters such as promoters for genes encoding glucocorticoid or estrogen receptors (inducible by treatment with the corresponding hormone), metallothionine promoter (inducible by treatment with various heavy metals), MX-1 promoter (inducible by interferon), the “GeneSwitch” mifepristone-regulatable system (Sirin et al., 2003, Gene, 323:67), the cumate inducible gene switch (WO 2002/088346), tetracycline-dependent regulatory systems, etc.

Conditional expression can also be achieved by using a site specific DNA recombinase. According to certain embodiments, the vector comprises at least one (typically two) site(s) for recombination mediated by a site specific recombinase. As used herein, the terms “recombinase” or “site specific recombinase” include excusive or integrative proteins, enzymes, co-factors or associated proteins that are involved in recombination reactions involving one or more recombination sites (e.g., two, three, four, five, seven, ten, twelve, fifteen, twenty, thirty, fifty, etc.), which may be wild-type proteins (see Landy, Current Opinion in Biotechnology 3:699-707 (1993)), or mutants, derivatives (e.g., fusion proteins containing the recombination protein sequences or fragments thereof), fragments, and variants thereof. Illustrative examples of recombinases suitable for use herein include, but are not limited to: Cre, Int, IHF, Xis, Flp, Fis, Hin, Gin, ÖC31, Cin, Tn3 resolvase, TndX, XerC, XerD, TnpX, Hjc, Gin, SpCCE1, and ParA.

The vectors may comprise one or more recombination sites for any of a wide variety of site specific recombinases. It is to be understood that the target site for a site specific recombinase is in addition to any site(s) required for integration of a vector, e.g., a retroviral vector or lentiviral vector. As used herein, the terms “recombination sequence,” “recombination site,” or “site specific recombination site” refer to a particular nucleic acid sequence to which a recombinase recognizes and binds.

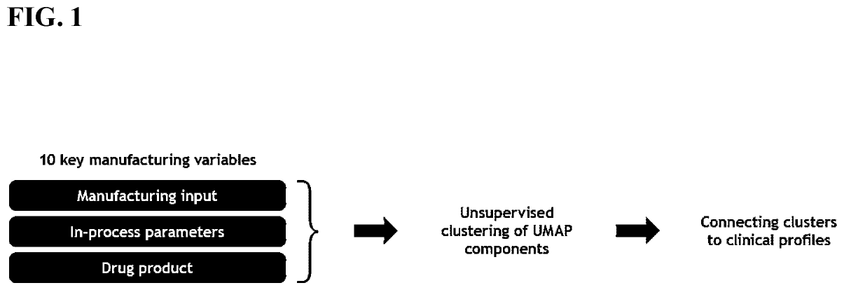

For example, one recombination site for Cre recombinase is loxP which is a 34 base pair sequence comprising two 13 base pair inverted repeats (serving as the recombinase binding sites) flanking an 8 base pair core sequence (see FIG. 1 of Sauer, B., Current Opinion in Biotechnology 5:521-527 (1994)). Other exemplary loxP sites include, but are not limited to: lox511 (Hoess et al., 1996; Bethke and Sauer, 1997), lox5171 (Lee and Saito, 1998), lox2272 (Lee and Saito, 1998), m2 (Langer et al., 2002), lox71 (Albert et al., 1995), and lox66 (Albert et al., 1995).

Suitable recognition sites for the FLP recombinase include, but are not limited to: FRT (McLeod, et al., 1996), F₁, F₂, F₃ (Schlake and Bode, 1994), F₄, F₅ (Schlake and Bode, 1994), FRT(LE) (Senecoff et al., 1988), FRT(RE) (Senecoff et al., 1988).

Other examples of recognition sequences are the attB, attP, attL, and attR sequences, which are recognized by the recombinase enzyme 6 Integrase, e.g., phi-c31. The öC31 SSR mediates recombination only between the heterotypic sites attB (34 bp in length) and attP (39 bp in length) (Groth et al., 2000). attB and attP, named for the attachment sites for the phage integrase on the bacterial and phage genomes, respectively, both contain imperfect inverted repeats that are likely bound by öC31 homodimers (Groth et al., 2000). The product sites, attL and attR, are effectively inert to further öC31-mediated recombination (Belteki et al., 2003), making the reaction irreversible. For catalyzing insertions, it has been found that attB-bearing DNA inserts into a genomic attP site more readily than an attP site into a genomic attB site (Thyagarajan et al., 2001; Belteki et al., 2003). Thus, typical strategies position by homologous recombination an attP-bearing “docking site” into a defined locus, which is then partnered with an attB-bearing incoming sequence for insertion.

As used herein, an “internal ribosome entry site” or “IRES” refers to an element that promotes direct internal ribosome entry to the initiation codon, such as ATG, of a cistron (a protein encoding region), thereby leading to the cap-independent translation of the gene. See, e.g., Jackson et al., 1990. Trends Biochem Sci 15(12):477-83) and Jackson and Kaminski. 1995. RNA 1(10):985-1000. In particular embodiments, the vectors contemplated herein include one or more polynucleotides-of-interest that encode one or more polypeptides. In particular embodiments, to achieve efficient translation of each of the plurality of polypeptides, the polynucleotide sequences can be separated by one or more IRES sequences or polynucleotide sequences encoding self-cleaving polypeptides.

As used herein, the term “Kozak sequence” refers to a short nucleotide sequence that greatly facilitates the initial binding of mRNA to the small subunit of the ribosome and increases translation. The consensus Kozak sequence is (GCC)RCCATGG, where R is a purine (A or G) (Kozak, 1986. Cell. 44(2):283-92, and Kozak, 1987. Nucleic Acids Res. 15(20):8125-48). In particular embodiments, the vectors contemplated herein comprise polynucleotides that have a consensus Kozak sequence and that encode a desired polypeptide, e.g., a CAR.

In some embodiments, a polynucleotide or cell harboring the polynucleotide utilizes a suicide gene, including an inducible suicide gene to reduce the risk of direct toxicity and/or uncontrolled proliferation. In specific aspects, the suicide gene is not immunogenic to the host harboring the polynucleotide or cell. A certain example of a suicide gene that may be used is caspase-9 or caspase-8 or cytosine deaminase. Caspase-9 can be activated using a specific chemical inducer of dimerization (CID).

In certain embodiments, vectors comprise gene segments that cause the immune effector cells of the present disclosure, e.g., T cells, to be susceptible to negative selection in vivo. By “negative selection” is meant that the infused cell can be eliminated as a result of a change in the in vivo condition of the individual. The negative selectable phenotype may result from the insertion of a gene that confers sensitivity to an administered agent, for example, a compound. Negative selectable genes are known in the art, and include, inter alia the following: the Herpes simplex virus type I thymidine kinase (HSV-I TK) gene (Wigler et al., Cell 11:223, 1977) which confers ganciclovir sensitivity; the cellular hypoxanthine phosphoribosyltransferase (HPRT) gene, the cellular adenine phosphoribosyltransferase (APRT) gene, and bacterial cytosine deaminase, (Mullen et al., Proc. Natl. Acad. Sci. USA. 89:33 (1992)).

In some embodiments, genetically modified immune effector cells, such as T cells, comprise a polynucleotide further comprising a positive marker that enables the selection of cells of the negative selectable phenotype in vitro. The positive selectable marker may be a gene which, upon being introduced into the host cell expresses a dominant phenotype permitting positive selection of cells carrying the gene. Genes of this type are known in the art, and include, inter alia, hygromycin-B phosphotransferase gene (hph) which confers resistance to hygromycin B, the amino glycoside phosphotransferase gene (neo or aph) from Tn5 which codes for resistance to the antibiotic G418, the dihydrofolate reductase (DHFR) gene, the adenosine deaminase gene (ADA), and the multi-drug resistance (MDR) gene.

Preferably, the positive selectable marker and the negative selectable element are linked such that loss of the negative selectable element necessarily also is accompanied by loss of the positive selectable marker. Even more preferably, the positive and negative selectable markers are fused so that loss of one obligatorily leads to loss of the other. An example of a fused polynucleotide that yields as an expression product a polypeptide that confers both the desired positive and negative selection features described above is a hygromycin phosphotransferase thymidine kinase fusion gene (HyTK). Expression of this gene yields a polypeptide that confers hygromycin B resistance for positive selection in vitro, and ganciclovir sensitivity for negative selection in vivo. See Lupton S. D., et al, Mol. and Cell. Biology 1 1:3374-3378, 1991. In addition, in certain embodiments, polynucleotides encoding the chimeric receptors are in retroviral vectors containing the fused gene, particularly those that confer hygromycin B resistance for positive selection in vitro, and ganciclovir sensitivity for negative selection in vivo, for example the HyTK retroviral vector described in Lupton, S. D. et al. (1991), supra. See also the publications of PCT US91/08442 and PCT/US94/05601, by S. D. Lupton, describing the use of bifunctional selectable fusion genes derived from fusing a dominant positive selectable markers with negative selectable markers.

Positive selectable markers can, for example, be derived from genes selected from the group consisting of hph, nco, and gpt, and negative selectable markers can, for example, be derived from genes selected from the group consisting of cytosine deaminase, HSV-I TK, VZV TK, HPRT, APRT and gpt. In specific embodiments, markers are bifunctional selectable fusion genes wherein the positive selectable marker is derived from hph or neo, and the negative selectable marker is derived from cytosine deaminase or a TK gene or selectable marker.

### C. T Cell Receptors (TCRs)

In some embodiments, the encoded recombinant receptor is a T cell receptor (TCR) or antigen-binding portion thereof that recognizes an peptide epitope or T cell epitope of a target polypeptide, such as an antigen of a tumor, viral or autoimmune protein.

In some embodiments, a “T cell receptor” or “TCR” is a molecule that contains a variable a and β chains (also known as TCRα and TCRβ, respectively) or a variable γ and δ chains (also known as TCRα and TCRβ, respectively), or antigen-binding portions thereof, and which is capable of specifically binding to a peptide bound to an MHC molecule. In some embodiments, the TCR is in the a form. Typically, TCRs that exist in αβ and γδ forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules.

Unless otherwise stated, the term “TCR” should be understood to encompass full TCRs as well as antigen-binding portions or antigen-binding fragments thereof. In some embodiments, the TCR is an intact or full-length TCR, including TCRs in the αβ form or γδ form. In some embodiments, the TCR is an antigen-binding portion that is less than a full-length TCR but that binds to a specific peptide bound in an MHC molecule, such as binds to an MHC-peptide complex. In some cases, an antigen-binding portion or fragment of a TCR can contain only a portion of the structural domains of a full-length or intact TCR, but yet is able to bind the peptide epitope, such as MHC-peptide complex, to which the full TCR binds. In some cases, an antigen-binding portion contains the variable domains of a TCR, such as variable a chain and variable f chain of a TCR, sufficient to form a binding site for binding to a specific MHC-peptide complex. Generally, the variable chains of a TCR contain complementarity determining regions involved in recognition of the peptide, MHC and/or MHC-peptide complex.

In some embodiments, the variable domains of the TCR contain hypervariable loops, or complementarity determining regions (CDRs), which generally are the primary contributors to antigen recognition and binding capabilities and specificity. In some embodiments, a CDR of a TCR or combination thereof forms all or substantially all of the antigen-binding site of a given TCR molecule. The various CDRs within a variable region of a TCR chain generally are separated by framework regions (FRs), which generally display less variability among TCR molecules as compared to the CDRs (see, e.g., Jores et al., Proc. Nat'l Acad. Sci. U.S.A. 87:9138, 1990; Chothia et al., EMBO J. 7:3745, 1988; see also Lefranc et al., Dev. Comp. Immunol. 27:55, 2003). In some embodiments, CDR3 is the main CDR responsible for antigen binding or specificity, or is the most important among the three CDRs on a given TCR variable region for antigen recognition, and/or for interaction with the processed peptide portion of the peptide-MHC complex. In some contexts, the CDR1 of the alpha chain can interact with the N-terminal part of certain antigenic peptides. In some contexts, CDR1 of the beta chain can interact with the C-terminal part of the peptide. In some contexts, CDR2 contributes most strongly to or is the primary CDR responsible for the interaction with or recognition of the MHC portion of the MHC-peptide complex. In some embodiments, the variable region of the p-chain can contain a further hypervariable region (CDR4 or HVR4), which generally is involved in superantigen binding and not antigen recognition (Kotb (1995) Clinical Microbiology Reviews, 8:411-426).

In some embodiments, a TCR also can contain a constant domain, a transmembrane domain and/or a short cytoplasmic tail (see, e.g., Janeway et al., Immunobiology: The Immune System in Health and Disease, 3rd Ed., Current Biology Publications, p. 4:33, 1997). In some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction.

In some embodiments, a TCR chain contains one or more constant domain. For example, the extracellular portion of a given TCR chain (e.g., α-chain or β-chain) can contain two immunoglobulin-like domains, such as a variable domain (e.g., Vα or Vβ; typically amino acids 1 to 116 based on Kabat numbering Kabat et al., “Sequences of Proteins of Immunological Interest, US Dept. Health and Human Services, Public Health Service National Institutes of Health, 1991, 5th ed.) and a constant domain (e.g., α-chain constant domain or Cα, typically positions 117 to 259 of the chain based on Kabat numbering or β chain constant domain or C_{β}, typically positions 117 to 295 of the chain based on Kabat) adjacent to the cell membrane. For example, in some cases, the extracellular portion of the TCR formed by the two chains contains two membrane-proximal constant domains, and two membrane-distal variable domains, which variable domains each contain CDRs. The constant domain of the TCR may contain short connecting sequences in which a cysteine residue forms a disulfide bond, thereby linking the two chains of the TCR. In some embodiments, a TCR may have an additional cysteine residue in each of the α and β chains, such that the TCR contains two disulfide bonds in the constant domains.

In some embodiments, the TCR chains contain a transmembrane domain. In some embodiments, the transmembrane domain is positively charged. In some cases, the TCR chain contains a cytoplasmic tail. In some cases, the structure allows the TCR to associate with other molecules like CD3 and subunits thereof. For example, a TCR containing constant domains with a transmembrane region may anchor the protein in the cell membrane and associate with invariant subunits of the CD3 signaling apparatus or complex. The intracellular tails of CD3 signaling subunits (e.g., CD3γ, CD3δ, CD3ε and CD3ζ) chains) contain one or more immunoreceptor tyrosine-based activation motif or ITAM that are involved in the signaling capacity of the TCR complex.

In some embodiments, the TCR may be a heterodimer of two chains a and R (or optionally γ and δ) or it may be a single chain TCR construct. In some embodiments, the TCR is a heterodimer containing two separate chains (α and β chains or γ and δ chains) that are linked, such as by a disulfide bond or disulfide bonds.

In some embodiments, the TCR can be generated from a known TCR sequence(s), such as sequences of Vα,β chains, for which a substantially full-length coding sequence is readily available. Methods for obtaining full-length TCR sequences, including V chain sequences, from cell sources are well known. In some embodiments, nucleic acids encoding the TCR can be obtained from a variety of sources, such as by polymerase chain reaction (PCR) amplification of TCR-encoding nucleic acids within or isolated from a given cell or cells, or synthesis of publicly available TCR DNA sequences.

In some embodiments, the TCR is obtained from a biological source, such as from cells such as from a T cell (e.g., cytotoxic T cell), T-cell hybridomas or other publicly available source. In some embodiments, the T-cells can be obtained from in vivo isolated cells. In some embodiments, the TCR is a thymically selected TCR. In some embodiments, the TCR is a neoepitope-restricted TCR. In some embodiments, the T-cells can be a cultured T-cell hybridoma or clone. In some embodiments, the TCR or antigen-binding portion thereof or antigen-binding fragment thereof can be synthetically generated from knowledge of the sequence of the TCR.

In some embodiments, the TCR is generated from a TCR identified or selected from screening a library of candidate TCRs against a target polypeptide antigen, or target T cell epitope thereof. TCR libraries can be generated by amplification of the repertoire of Vα and Vβ from T cells isolated from a subject, including cells present in PBMCs, spleen or other lymphoid organ. In some cases, T cells can be amplified from tumor-infiltrating lymphocytes (TILs). In some embodiments, TCR libraries can be generated from CD4+ or CD8+ cells. In some embodiments, the TCRs can be amplified from a T cell source of a normal of healthy subject, i.e. normal TCR libraries. In some embodiments, the TCRs can be amplified from a T cell source of a diseased subject, i.e. diseased TCR libraries. In some embodiments, degenerate primers are used to amplify the gene repertoire of Vα and Vβ, such as by RT-PCR in samples, such as T cells, obtained from humans. In some embodiments, scTv libraries can be assembled from naive Vα and Vβ libraries in which the amplified products are cloned or assembled to be separated by a linker. Depending on the source of the subject and cells, the libraries can be HLA allele-specific. Alternatively, in some embodiments, TCR libraries can be generated by mutagenesis or diversification of a parent or scaffold TCR molecule. In some aspects, the TCRs are subjected to directed evolution, such as by mutagenesis, e.g., of the a or β chain. In some aspects, particular residues within CDRs of the TCR are altered. In some embodiments, selected TCRs can be modified by affinity maturation. In some embodiments, antigen-specific T cells may be selected, such as by screening to assess CTL activity against the peptide. In some aspects, TCRs, e.g., present on the antigen-specific T cells, may be selected, such as by binding activity, e.g., particular affinity or avidity for the antigen.

In some embodiments, the genetically engineered antigen receptors include recombinant T cell receptors (TCRs) and/or TCRs cloned from naturally occurring T cells. In some embodiments, a high-affinity T cell clone for a target antigen (e.g., a cancer antigen) is identified, isolated from a patient, and introduced into the cells. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al. (2009) Clin Cancer Res. 15:169-180 and Cohen et al. (2005) J Immunol. 175:5799-5808. In some embodiments, phage display is used to isolate TCRs against a target antigen (see, e.g., Varela-Rohena et al. (2008) Nat Med. 14:1390-1395 and Li (2005) Nat Biotechnol. 23:349-354.

In some embodiments, the TCR or antigen-binding portion thereof is one that has been modified or engineered. In some embodiments, directed evolution methods are used to generate TCRs with altered properties, such as with higher affinity for a specific MHC-peptide complex. In some embodiments, directed evolution is achieved by display methods including, but not limited to, yeast display (Holler et al. (2003) Nat Immunol, 4, 55-62; Holler et al. (2000) Proc Natl Acad Sci USA, 97, 5387-92), phage display (Li et al. (2005) Nat Biotechnol, 23, 349-54), or T cell display (Chervin et al. (2008) J Immunol Methods, 339, 175-84). In some embodiments, display approaches involve engineering, or modifying, a known, parent or reference TCR. For example, in some cases, a wild-type TCR can be used as a template for producing mutagenized TCRs in which in one or more residues of the CDRs are mutated, and mutants with an desired altered property, such as higher affinity for a desired target antigen, are selected.

In some embodiments, peptides of a target polypeptide for use in producing or generating a TCR of interest are known or can be readily identified by a skilled artisan. In some embodiments, peptides suitable for use in generating TCRs or antigen-binding portions can be determined based on the presence of an HLA-restricted motif in a target polypeptide of interest, such as a target polypeptide described below. In some embodiments, peptides are identified using available computer prediction models. In some embodiments, for predicting MHC class I binding sites, such models include, but are not limited to, ProPred1 (Singh and Raghava (2001) Bioinformatics 17(12):1236-1237, and SYFPEITHI (see Schuler et al. (2007) Immunoinformatics Methods in Molecular Biology, 409(1): 75-93 2007). In some embodiments, the MHC-restricted epitope is HLA-A0201, which is expressed in approximately 39-46% of all Caucasians and therefore, represents a suitable choice of MHC antigen for use preparing a TCR or other MHC-peptide binding molecule.

HLA-A0201-binding motifs and the cleavage sites for proteasomes and immune-proteasomes using computer prediction models are known. For predicting MHC class I binding sites, such models include, but are not limited to, ProPred1 (described in more detail in Singh and Raghava, ProPred: prediction of HLA-DR binding sites. BIOINFORMATICS 17(12):1236-1237 2001), and SYFPEITHI (see Schuler et al. SYFPEITHI, Database for Searching and T-Cell Epitope Prediction. in Immunoinformatics Methods in Molecular Biology, vol 409(1): 75-93 2007).

In some embodiments, the TCR or antigen binding portion thereof may be a recombinantly produced natural protein or mutated form thereof in which one or more property, such as binding characteristic, has been altered. In some embodiments, a TCR may be derived from one of various animal species, such as human, mouse, rat, or other mammal. A TCR may be cell-bound or in soluble form. In some embodiments, for purposes of the provided methods, the TCR is in cell-bound form expressed on the surface of a cell.

In some embodiments, the TCR is a full-length TCR. In some embodiments, the TCR is an antigen-binding portion. In some embodiments, the TCR is a dimeric TCR (dTCR). In some embodiments, the TCR is a single-chain TCR (sc-TCR). In some embodiments, a dTCR or scTCR have the structures as described in WO 03/020763, WO 04/033685, WO2011/044186.

In some embodiments, the TCR contains a sequence corresponding to the transmembrane sequence. In some embodiments, the TCR does contain a sequence corresponding to cytoplasmic sequences. In some embodiments, the TCR is capable of forming a TCR complex with CD3. In some embodiments, any of the TCRs, including a dTCR or scTCR, can be linked to signaling domains that yield an active TCR on the surface of a T cell. In some embodiments, the TCR is expressed on the surface of cells.

In some embodiments a dTCR contains a first polypeptide wherein a sequence corresponding to a TCR α chain variable region sequence is fused to the N terminus of a sequence corresponding to a TCR α chain constant region extracellular sequence, and a second polypeptide wherein a sequence corresponding to a TCR β chain variable region sequence is fused to the N terminus a sequence corresponding to a TCR β chain constant region extracellular sequence, the first and second polypeptides being linked by a disulfide bond. In some embodiments, the bond can correspond to the native inter-chain disulfide bond present in native dimeric αβ TCRs. In some embodiments, the interchain disulfide bonds are not present in a native TCR. For example, in some embodiments, one or more cysteines can be incorporated into the constant region extracellular sequences of dTCR polypeptide pair. In some cases, both a native and a non-native disulfide bond may be desirable. In some embodiments, the TCR contains a transmembrane sequence to anchor to the membrane.

In some embodiments, a dTCR contains a TCR α chain containing a variable a domain, a constant α domain and a first dimerization motif attached to the C-terminus of the constant α domain, and a TCR β chain comprising a variable β domain, a constant β domain and a first dimerization motif attached to the C-terminus of the constant β domain, wherein the first and second dimerization motifs easily interact to form a covalent bond between an amino acid in the first dimerization motif and an amino acid in the second dimerization motif linking the TCR α chain and TCR β chain together.

In some embodiments, the TCR is a scTCR. Typically, a scTCR can be generated using methods known, See, e.g., Soo Hoo, W. F. et al. PNAS (USA) 89, 4759 (1992); Wilfing, C. and Plückthun, A., J. Mol. Biol. 242, 655 (1994); Kurucz, I. et al. PNAS (USA) 90 3830 (1993); International published PCT Nos. WO 96/13593, WO 96/18105, WO99/60120, WO99/18129, WO 03/020763, WO2011/044186; and Schlueter, C. J. et al. J. Mol. Biol. 256, 859 (1996). In some embodiments, a scTCR contains an introduced non-native disulfide interchain bond to facilitate the association of the TCR chains (see, e.g., International published PCT No. WO 03/020763). In some embodiments, a scTCR is a non-disulfide linked truncated TCR in which heterologous leucine zippers fused to the C-termini thereof facilitate chain association (see, e.g., International published PCT No. WO99/60120). In some embodiments, a scTCR contain a TCRα variable domain covalently linked to a TCRβ variable domain via a peptide linker (see, e.g., International published PCT No. WO99/18129).

In some embodiments, a scTCR contains a first segment constituted by an amino acid sequence corresponding to a TCR α chain variable region, a second segment constituted by an amino acid sequence corresponding to a TCR β chain variable region sequence fused to the N terminus of an amino acid sequence corresponding to a TCR β chain constant domain extracellular sequence, and a linker sequence linking the C terminus of the first segment to the N terminus of the second segment.

In some embodiments, a scTCR contains a first segment constituted by an α chain variable region sequence fused to the N terminus of an α chain extracellular constant domain sequence, and a second segment constituted by a β chain variable region sequence fused to the N terminus of a sequence β chain extracellular constant and transmembrane sequence, and, optionally, a linker sequence linking the C terminus of the first segment to the N terminus of the second segment.

In some embodiments, a scTCR contains a first segment constituted by a TCR β chain variable region sequence fused to the N terminus of a β chain extracellular constant domain sequence, and a second segment constituted by an α chain variable region sequence fused to the N terminus of a sequence α chain extracellular constant and transmembrane sequence, and, optionally, a linker sequence linking the C terminus of the first segment to the N terminus of the second segment.

In some embodiments, the linker of a scTCRs that links the first and second TCR segments can be any linker capable of forming a single polypeptide strand, while retaining TCR binding specificity. In some embodiments, the linker sequence may, for example, have the formula -P-AA-P- wherein P is proline and AA represents an amino acid sequence wherein the amino acids are glycine and serine. In some embodiments, the first and second segments are paired so that the variable region sequences thereof are orientated for such binding. Hence, in some cases, the linker has a sufficient length to span the distance between the C terminus of the first segment and the N terminus of the second segment, or vice versa, but is not too long to block or reduces bonding of the scTCR to the target ligand. In some embodiments, the linker can contain from or from about 10 to 45 amino acids, such as 10 to 30 amino acids or 26 to 41 amino acids residues, for example 29, 30, 31 or 32 amino acids. In some embodiments, the linker has the formula -PGGG-(SGGGG)₅-P- wherein P is proline, G is glycine and S is serine. In some embodiments, the linker has the sequence GSADDAKKDAAKKDGKS.

In some embodiments, the scTCR contains a covalent disulfide bond linking a residue of the immunoglobulin region of the constant domain of the α chain to a residue of the immunoglobulin region of the constant domain of the β chain. In some embodiments, the interchain disulfide bond in a native TCR is not present. For example, in some embodiments, one or more cysteines can be incorporated into the constant region extracellular sequences of the first and second segments of the scTCR polypeptide. In some cases, both a native and a non-native disulfide bond may be desirable.

In some embodiments of a dTCR or scTCR containing introduced interchain disulfide bonds, the native disulfide bonds are not present. In some embodiments, the one or more of the native cysteines forming a native interchain disulfide bonds are substituted to another residue, such as to a serine or alanine. In some embodiments, an introduced disulfide bond can be formed by mutating non-cysteine residues on the first and second segments to cysteine. Exemplary non-native disulfide bonds of a TCR are described in published International PCT No. WO2006/000830.

In some embodiments, the TCR or antigen-binding fragment thereof exhibits an affinity with an equilibrium binding constant for a target antigen of between or between about 10⁻⁵ and 10⁻¹² M and all individual values and ranges therein. In some embodiments, the target antigen is an MHC-peptide complex or ligand.

In some embodiments, nucleic acid or nucleic acids encoding a TCR, such as a and R chains, can be amplified by PCR, cloning or other suitable means and cloned into a suitable expression vector or vectors. The expression vector can be any suitable recombinant expression vector, and can be used to transform or transfect any suitable host. Suitable vectors include those designed for propagation and expansion or for expression or both, such as plasmids and viruses.

In some embodiments, the recombinant expression vectors can be prepared using standard recombinant DNA techniques. In some embodiments, vectors can contain regulatory sequences, such as transcription and translation initiation and termination codons, which are specific to the type of host (e.g., bacterium, fungus, plant, or animal) into which the vector is to be introduced, as appropriate and taking into consideration whether the vector is DNA- or RNA-based. In some embodiments, the vector can contain a nonnative promoter operably linked to the nucleotide sequence encoding the TCR or antigen-binding portion (or other MHC-peptide binding molecule). In some embodiments, the promoter can be a non-viral promoter or a viral promoter, such as a cytomegalovirus (CMV) promoter, an SV40 promoter, an RSV promoter, and a promoter found in the long-terminal repeat of the murine stem cell virus. Other known promoters also are contemplated.

In some embodiments, after the T-cell clone is obtained, the TCR alpha and beta chains are isolated and cloned into a gene expression vector. In some embodiments, the TCR alpha and beta genes are linked via a picornavirus 2A ribosomal skip peptide so that both chains are co-expressed. In some embodiments, genetic transfer of the TCR is accomplished via retroviral or lentiviral vectors, or via transposons (see, e.g., Baum et al. (2006) Molecular Therapy: The Journal of the American Society of Gene Therapy. 13:1050-1063; Frecha et al. (2010) Molecular Therapy: The Journal of the American Society of Gene Therapy. 18:1748-1757; and Hackett et al. (2010) Molecular Therapy: The Journal of the American Society of Gene Therapy. 18:674-683.

In some embodiments, to generate a vector encoding a TCR, the α and β chains are PCR amplified from total cDNA isolated from a T cell clone expressing the TCR of interest and cloned into an expression vector. In some embodiments, the α and β chains are cloned into the same vector. In some embodiments, the α and β chains are cloned into different vectors. In some embodiments, the generated α and β chains are incorporated into a retroviral, e.g., lentiviral, vector.

### IV. Methods of Administration

In some aspects, the T cell therapy, also referred to herein as a therapeutic cell composition, comprising engineered cells, e.g., engineered CD4+ and CD8+ cells, can be used in connection with a method of treatment, e.g., including administering any of the engineered cells or compositions containing engineered cells that have been assessed using the methods provided herein.

In some embodiments, the engineered cells expressing a recombinant receptor or compositions comprising the same, are useful in a variety of therapeutic, diagnostic and prophylactic indications. For example, the engineered cells or compositions comprising the engineered cells, e.g., therapeutic cell compositions, are useful in treating a variety of diseases and disorders in a subject. Methods and uses include therapeutic methods and uses, for example, involving administration of the engineered cells, or compositions containing the same, to a subject having a disease, condition, or disorder, such as a tumor or cancer. In some embodiments, the engineered cells or compositions assessed or evaluated using the embodiments provided herein are administered in an effective amount to effect treatment of the disease or disorder. Uses include uses of the engineered cells or compositions in such methods and treatments, and in the preparation of a medicament in order to carry out such therapeutic methods. In some embodiments, the methods, e.g., therapeutic methods, are carried out by administering the assessed or evaluated engineered cells, or compositions comprising the same, to the subject having or suspected of having the disease or condition. In some embodiments, these methods thereby treat the disease or condition or disorder in the subject.

In some aspects, the engineered cells or engineered cell composition can be administered to a subject, such as a subject that has a disease or disorder. Methods for administration of cells for adoptive cell therapy are known and may be used in connection with the provided methods and compositions. For example, adoptive T cell therapy methods are described, e.g., in US Pat. App. Pub. No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

The disease or condition that is treated can be any in which expression of an antigen is associated with and/or involved in the etiology of a disease condition or disorder, e.g., causes, exacerbates or otherwise is involved in such disease, condition, or disorder. Exemplary diseases and conditions can include diseases or conditions associated with malignancy or transformation of cells (e.g., cancer), autoimmune or inflammatory disease, or an infectious disease, e.g., caused by a bacterial, viral or other pathogen. Exemplary antigens, which include antigens associated with various diseases and conditions that can be treated, are described above. In particular embodiments, the chimeric antigen receptor or transgenic TCR specifically binds to an antigen associated with the disease or condition.

Among the diseases, conditions, and disorders are tumors, including solid tumors, hematologic malignancies, and melanomas, and including localized and metastatic tumors, infectious diseases, such as infection with a virus or other pathogen, e.g., HIV, HCV, HBV, CMV, HPV, and parasitic disease, and autoimmune and inflammatory diseases. In some embodiments, the disease, disorder or condition is a tumor, cancer, malignancy, neoplasm, or other proliferative disease or disorder. Such diseases include but are not limited to leukemia, lymphoma, e.g., acute myeloid (or myelogenous) leukemia (AML), chronic myeloid (or myelogenous) leukemia (CML), acute lymphocytic (or lymphoblastic) leukemia (ALL), chronic lymphocytic leukemia (CLL), hairy cell leukemia (HCL), small lymphocytic lymphoma (SLL), Mantle cell lymphoma (MCL), Marginal zone lymphoma, Burkitt lymphoma, Hodgkin lymphoma (HL), non-Hodgkin lymphoma (NHL), Anaplastic large cell lymphoma (ALCL), follicular lymphoma, refractory follicular lymphoma, diffuse large B-cell lymphoma (DLBCL) and multiple myeloma (MM). In some embodiments, disease or condition is a B cell malignancy selected from among acute lymphoblastic leukemia (ALL), adult ALL, chronic lymphoblastic leukemia (CLL), non-Hodgkin lymphoma (NHL), and Diffuse Large B-Cell Lymphoma (DLBCL). In some embodiments, the disease or condition is NHL and the NHL is selected from the group consisting of aggressive NHL, diffuse large B cell lymphoma (DLBCL), NOS (de novo and transformed from indolent), primary mediastinal large B cell lymphoma (PMBCL), T cell/histocyte-rich large B cell lymphoma (TCHRBCL), Burkitt's lymphoma, mantle cell lymphoma (MCL), and/or follicular lymphoma (FL), optionally, follicular lymphoma Grade 3B (FL3B).

In some embodiments, the disease or condition is an infectious disease or condition, such as, but not limited to, viral, retroviral, bacterial, and protozoal infections, immunodeficiency, Cytomegalovirus (CMV), Epstein-Barr virus (EBV), adenovirus, BK polyomavirus. In some embodiments, the disease or condition is an autoimmune or inflammatory disease or condition, such as arthritis, e.g., rheumatoid arthritis (RA), Type I diabetes, systemic lupus erythematosus (SLE), inflammatory bowel disease, psoriasis, scleroderma, autoimmune thyroid disease, Grave's disease, Crohn's disease, multiple sclerosis, asthma, and/or a disease or condition associated with transplant.

In some embodiments, the antigen associated with the disease or disorder is or includes αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD133, CD138, CD171, epidermal growth factor protein (EGFR), truncated epidermal growth factor protein (tEGFR), type III epidermal growth factor receptor mutation (EGFR vIII), epithelial glycoprotein 2 (EPG-2), epithelial glycoprotein 40 (EPG-40), ephrinB2, ephrine receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G protein-coupled receptor class C group 5 member D (GPRC5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha (IL-22Rα), IL-13 receptor alpha 2 (IL-13Rα2), kinase insert domain receptor (kdr), kappa light chain, L1 cell adhesion molecule (L1-CAM), CE7 epitope of Li-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT) vascular endothelial growth factor receptor (VEGFR), vascular endothelial growth factor receptor 2 (VEGFR2), Wilms Tumor 1 (WT-1), a pathogen-specific or pathogen-expressed antigen, or an antigen associated with a universal tag, and/or biotinylated molecules, and/or molecules expressed by HIV, HCV, HBV or other pathogens. Antigens targeted by the receptors in some embodiments include antigens associated with a B cell malignancy, such as any of a number of known B cell marker. In some embodiments, the antigen is or includes CD20, CD19, CD22, ROR1, CD45, CD21, CD5, CD33, Igkappa, Iglambda, CD79a, CD79b or CD30. In some embodiments, the antigen is or includes a pathogen-specific or pathogen-expressed antigen, such as a viral antigen (e.g., a viral antigen from HIV, HCV, HBV), bacterial antigens, and/or parasitic antigens.

In some embodiments, the antibody or an antigen-binding fragment (e.g., scFv or V_{H} domain) specifically recognizes an antigen, such as CD19. In some embodiments, the antibody or antigen-binding fragment is derived from, or is a variant of, antibodies or antigen-binding fragment that specifically binds to CD19. In some embodiments, the cell therapy, e.g., adoptive T cell therapy, is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the disease or condition is a B cell malignancy. In some embodiments, the B cell malignancy is a leukemia or a lymphoma. In some aspects, the disease or condition is acute lymphoblastic leukemia (ALL), adult ALL, chronic lymphoblastic leukemia (CLL), non-Hodgkin lymphoma (NHL), or Diffuse Large B-Cell Lymphoma (DLBCL). In some cases, the disease or condition is an NHL, such as or including an NHL that is an aggressive NHL, diffuse large B cell lymphoma (DLBCL), NOS (de novo and transformed from indolent), primary mediastinal large B cell lymphoma (PMBCL), T cell/histocyte-rich large B cell lymphoma (TCHRBCL), Burkitt's lymphoma, mantle cell lymphoma (MCL), and/or follicular lymphoma (FL), optionally, follicular lymphoma Grade 3B (FL3B). In some aspects, the recombinant receptor, such as a CAR, specifically binds to an antigen associated with the disease or condition or expressed in cells of the environment of a lesion associated with the B cell malignancy. Antigens targeted by the receptors in some embodiments include antigens associated with a B cell malignancy, such as any of a number of known B cell marker. In some embodiments, the antigen targeted by the receptor is CD20, CD19, CD22, ROR1, CD45, CD21, CD5, CD33, Igkappa, Iglambda, CD79a, CD79b or CD30, or combinations thereof.

In some embodiments, the disease or condition is a myeloma, such as a multiple myeloma. In some aspects, the recombinant receptor, such as a CAR, specifically binds to an antigen associated with the disease or condition or expressed in cells of the environment of a lesion associated with the multiple myeloma. Antigens targeted by the receptors in some embodiments include antigens associated with multiple myeloma. In some aspects, the antigen, e.g., the second or additional antigen, such as the disease-specific antigen and/or related antigen, is expressed on multiple myeloma, such as B cell maturation antigen (BCMA), G protein-coupled receptor class C group 5 member D (GPRC5D), CD38 (cyclic ADP ribose hydrolase), CD138 (syndecan-1, syndecan, SYN-1), CS-1 (CS1, CD2 subset 1, CRACC, SLAMF7, CD319, and 19A24), BAFF-R, TACI and/or FcRH5. Other exemplary multiple myeloma antigens include CD56, TIM-3, CD33, CD123, CD44, CD20, CD40, CD74, CD200, EGFR, 02-Microglobulin, HM1.24, IGF-1R, IL-6R, TRAIL-RI, and the activin receptor type IIA (ActRIIA). See Benson and Byrd, J. Clin. Oncol. (2012) 30(16): 2013-15; Tao and Anderson, Bone Marrow Research (2011):924058; Chu et al., Leukemia (2013) 28(4):917-27; Garfall et al., Discov Med. (2014) 17(91):37-46. In some embodiments, the antigens include those present on lymphoid tumors, myeloma, AIDS-associated lymphoma, and/or post-transplant lymphoproliferations, such as CD38. Antibodies or antigen-binding fragments directed against such antigens are known and include, for example, those described in U.S. Pat. Nos. 8,153,765; 8,603,477, 8,008,450; U.S. Pub. No. US20120189622 or US20100260748; and/or International PCT Publication Nos. WO2006099875, WO2009080829 or WO2012092612 or WO2014210064. In some embodiments, such antibodies or antigen-binding fragments thereof (e.g., scFv) are contained in multispecific antibodies, multispecific chimeric receptors, such as multispecific CARs, and/or multispecific cells.

In some embodiments, the disease or disorder is associated with expression of G protein-coupled receptor class C group 5 member D (GPRC5D) and/or expression of B cell maturation antigen (BCMA).

In some embodiments, the disease or disorder is a B cell-related disorder. In some of any of the provided embodiments of the provided methods, the disease or disorder associated with BCMA is an autoimmune disease or disorder. In some of any of the provided embodiments of the provided methods, the autoimmune disease or disorder is systemic lupus erythematosus (SLE), lupus nephritis, inflammatory bowel disease, rheumatoid arthritis, ANCA associated vasculitis, idiopathic thrombocytopenia purpura (ITP), thrombotic thrombocytopenia purpura (TTP), autoimmune thrombocytopenia, Chagas' disease, Grave's disease, Wegener's granulomatosis, poly-arteritis nodosa, Sjogren's syndrome, pemphigus vulgaris, scleroderma, multiple sclerosis, psoriasis, IgA nephropathy, IgM polyneuropathies, vasculitis, diabetes mellitus, Reynaud's syndrome, anti-phospholipid syndrome, Goodpasture's disease, Kawasaki disease, autoimmune hemolytic anemia, myasthenia gravis, or progressive glomerulonephritis.

In some embodiments, the disease or disorder is a cancer. In some embodiments, the cancer is a GPRC5D-expressing cancer. In some embodiments, the cancer is a plasma cell malignancy and the plasma cell malignancy is multiple myeloma (MM) or plasmacytoma. In some embodiments, the cancer is multiple myeloma (MM). In some embodiments, the cancer is a relapsed/refractory multiple myeloma.

In some embodiments, the cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

The cells can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration of the cells. In some embodiments, it is administered by multiple bolus administrations of the cells, for example, over a period of no more than 3 days, or by continuous infusion administration of the cells. In some embodiments, administration of the cell dose or any additional therapies, e.g., the lymphodepleting therapy, intervention therapy and/or combination therapy, is carried out via outpatient delivery.

For the prevention or treatment of disease, the appropriate dosage may depend on the type of disease to be treated, the type of cells or recombinant receptors, the severity and course of the disease, whether the cells are administered for preventive or therapeutic purposes, previous therapy, the subject's clinical history and response to the cells, and the discretion of the attending physician. The compositions and cells are in some embodiments suitably administered to the subject at one time or over a series of treatments.

In some embodiments, the cells are administered as part of a combination treatment, such as simultaneously with or sequentially with, in any order, another therapeutic intervention, such as an antibody or engineered cell or receptor or agent, such as a cytotoxic or therapeutic agent. The cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, the one or more additional agents include a cytokine, such as IL-2, for example, to enhance persistence. In some embodiments, the methods comprise administration of a chemotherapeutic agent.

In some embodiments, the subject is administered a chemotherapeutic agent, e.g., a conditioning chemotherapeutic agent, for example, to reduce tumor burden prior to the administration.

Preconditioning subjects with immunodepleting (e.g., lymphodepleting) therapies in some aspects can improve the effects of adoptive cell therapy (ACT).

Thus, in some embodiments, the subject is administered a preconditioning agent, such as a lymphodepleting or chemotherapeutic agent, such as cyclophosphamide, fludarabine, or combinations thereof, to a subject prior to the initiation of the cell therapy. For example, the subject may be administered a preconditioning agent at least 2 days prior, such as at least 3, 4, 5, 6, or 7 days prior, to the initiation of the cell therapy. In some embodiments, the subject is administered a preconditioning agent no more than 7 days prior, such as no more than 6, 5, 4, 3, or 2 days prior, to the initiation of the cell therapy.

In some embodiments, the subject is preconditioned with cyclophosphamide at a dose between or between about 20 mg/kg and 100 mg/kg, such as between or between about 40 mg/kg and 80 mg/kg. In some aspects, the subject is preconditioned with or with about 60 mg/kg of cyclophosphamide. In some embodiments, the cyclophosphamide can be administered in a single dose or can be administered in a plurality of doses, such as given daily, every other day or every three days. In some embodiments, the cyclophosphamide is administered once daily for one or two days. In some embodiments, where the lymphodepleting agent comprises cyclophosphamide, the subject is administered cyclophosphamide at a dose between or between about 100 mg/m² and 500 mg/m², such as between or between about 200 mg/m² and 400 mg/m², or 250 mg/m² and 350 mg/m², inclusive. In some instances, the subject is administered about 300 mg/m² of cyclophosphamide. In some embodiments, the cyclophosphamide can be administered in a single dose or can be administered in a plurality of doses, such as given daily, every other day or every three days. In some embodiments, cyclophosphamide is administered daily, such as for 1-5 days, for example, for 3 to 5 days. In some instances, the subject is administered about 300 mg/m² of cyclophosphamide, daily for 3 days, prior to initiation of the cell therapy.

In some embodiments, where the lymphodepleting agent comprises fludarabine, the subject is administered fludarabine at a dose between or between about 1 mg/m² and 100 mg/m², such as between or between about 10 mg/m² and 75 mg/m², 15 mg/m² and 50 mg/m², 20 mg/m² and 40 mg/m², or 24 mg/m² and 35 mg/m², inclusive. In some instances, the subject is administered about 30 mg/m² of fludarabine. In some embodiments, the fludarabine can be administered in a single dose or can be administered in a plurality of doses, such as given daily, every other day or every three days. In some embodiments, fludarabine is administered daily, such as for 1-5 days, for example, for 3 to 5 days. In some instances, the subject is administered about 30 mg/m² of fludarabine, daily for 3 days, prior to initiation of the cell therapy.

In some embodiments, the lymphodepleting agent comprises a combination of agents, such as a combination of cyclophosphamide and fludarabine. Thus, the combination of agents may include cyclophosphamide at any dose or administration schedule, such as those described above, and fludarabine at any dose or administration schedule, such as those described above. For example, in some aspects, the subject is administered 60 mg/kg (˜2 g/m²) of cyclophosphamide and 3 to 5 doses of 25 mg/m² fludarabine prior to the first or subsequent dose.

Following administration of the cells, the biological activity of the engineered cell populations in some embodiments is measured, e.g., by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable known methods, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells is measured by assaying expression and/or secretion of one or more cytokines, such as CD107a, IFNγ, IL-2, and TNF. In some aspects the biological activity is measured by assessing clinical outcome, such as reduction in tumor burden or load.

In certain embodiments, the engineered cells are further modified in any number of ways, such that their therapeutic or prophylactic efficacy is increased. For example, the engineered CAR or TCR expressed by the population can be conjugated either directly or indirectly through a linker to a targeting moiety. The practice of conjugating compounds, e.g., the CAR or TCR, to targeting moieties is known. See, for instance, Wadwa et al., J. Drug Targeting 3: 1 1 1 (1995), and U.S. Pat. No. 5,087,616.

In some embodiments, the cells are administered as part of a combination treatment, such as simultaneously with or sequentially with, in any order, another therapeutic intervention, such as an antibody or engineered cell or receptor or agent, such as a cytotoxic or therapeutic agent. The cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, the one or more additional agent includes a cytokine, such as IL-2, for example, to enhance persistence.

### A. Dosing

In some embodiments, a dose of cells is administered to subjects in accord with the provided methods, and/or with the provided articles of manufacture or compositions. In some embodiments, the size or timing of the doses is determined as a function of the particular disease or condition in the subject. In some cases, the size or timing of the doses for a particular disease in view of the provided description may be empirically determined.

In some embodiments, the dose of cells comprises between at or about 2×10⁵ of the cells/kg and at or about 2×10⁶ of the cells/kg, such as between at or about 4×10⁵ of the cells/kg and at or about 1×10⁶ of the cells/kg or between at or about 6×10⁵ of the cells/kg and at or about 8×10⁵ of the cells/kg. In some embodiments, the dose of cells comprises no more than 2×10⁵ of the cells (e.g., antigen-expressing, such as CAR-expressing cells) per kilogram body weight of the subject (cells/kg), such as no more than at or about 3×10⁵ cells/kg, no more than at or about 4×10⁵ cells/kg, no more than at or about 5×10⁵ cells/kg, no more than at or about 6×10⁵ cells/kg, no more than at or about 7×10⁵ cells/kg, no more than at or about 8×10⁵ cells/kg, no more than at or about 9×10⁵ cells/kg, no more than at or about 1×10⁶ cells/kg, or no more than at or about 2×10⁶ cells/kg. In some embodiments, the dose of cells comprises at least or at least about or at or about 2×10⁵ of the cells (e.g., antigen-expressing, such as CAR-expressing cells) per kilogram body weight of the subject (cells/kg), such as at least or at least about or at or about 3×10⁵ cells/kg, at least or at least about or at or about 4×10⁵ cells/kg, at least or at least about or at or about 5×10⁵ cells/kg, at least or at least about or at or about 6×10⁵ cells/kg, at least or at least about or at or about 7×10⁵ cells/kg, at least or at least about or at or about 8×10⁵ cells/kg, at least or at least about or at or about 9×10⁵ cells/kg, at least or at least about or at or about 1×10⁶ cells/kg, or at least or at least about or at or about 2×10⁶ cells/kg.

In certain embodiments, the cells, or individual populations of sub-types of cells, are administered to the subject at a range of at or about 0.1 million to at or about 100 billion cells and/or that amount of cells per kilogram of body weight of the subject, such as, e.g., at or about 0.1 million to at or about 50 billion cells (e.g., at or about 5 million cells, at or about 25 million cells, at or about 500 million cells, at or about 1 billion cells, at or about 5 billion cells, at or about 20 billion cells, at or about 30 billion cells, at or about 40 billion cells, or a range defined by any two of the foregoing values), at or about 1 million to at or about 50 billion cells (e.g., at or about 5 million cells, at or about 25 million cells, at or about 500 million cells, at or about 1 billion cells, at or about 5 billion cells, at or about 20 billion cells, at or about 30 billion cells, at or about 40 billion cells, or a range defined by any two of the foregoing values), such as at or about 10 million to at or about 100 billion cells (e.g., at or about 20 million cells, at or about 30 million cells, at or about 40 million cells, at or about 60 million cells, at or about 70 million cells, at or about 80 million cells, at or about 90 million cells, at or about 10 billion cells, at or about 25 billion cells, at or about 50 billion cells, at or about 75 billion cells, at or about 90 billion cells, or a range defined by any two of the foregoing values), and in some cases at or about 100 million cells to at or about 50 billion cells (e.g., at or about 120 million cells, at or about 250 million cells, at or about 350 million cells, about 450 million cells, at or about 650 million cells, at or about 800 million cells, at or about 900 million cells, at or about 3 billion cells, at or about 30 billion cells, at or about 45 billion cells) or any value in between these ranges and/or per kilogram of body weight of the subject. Dosages may vary depending on attributes particular to the disease or disorder and/or patient and/or other treatments.

In some embodiments, the dose of cells is a flat dose of cells or fixed dose of cells such that the dose of cells is not tied to or based on the body surface area or weight of a subject. In some embodiments, such values refer to numbers of recombinant receptor-expressing cells; in other embodiments, they refer to number of T cells or PBMCs or total cells administered.

In some embodiments, for example, where the subject is a human, the dose includes fewer than about 5×10⁸ total recombinant receptor (e.g., CAR)-expressing cells, T cells, or peripheral blood mononuclear cells (PBMCs), e.g., in the range of about 1×10⁶ to 5×10⁸ such cells, such as 2×10⁶, 5×10⁶, 1×10⁷, 5×10⁷, 1×10⁸, or 5×10⁸, at or about 1×10⁶ to at or about 5×10⁸ such cells, such as at or about 2×10⁶, 5×10⁶, 1×10⁷, 5×10⁷, 1×10⁸, 1.5×10⁸, or 5×10⁸ total such cells, or the range between any two of the foregoing values. In some embodiments, for example, where the subject is a human, the dose includes more than at or about 1×10⁶ total recombinant receptor (e.g., CAR)-expressing cells, T cells, or peripheral blood mononuclear cells (PBMCs) and fewer than at or about 2×10⁹ total recombinant receptor (e.g., CAR)-expressing cells, T cells, or peripheral blood mononuclear cells (PBMCs), e.g., in the range of at or about 2.5×10⁷ to at or about 1.2×10⁹ such cells, such as at or about 2.5×10⁷, 5×10⁷, 1×10⁸, 1.5×10⁸, 8×10⁸, or 1.2×10⁹ total such cells, or the range between any two of the foregoing values.

In some embodiments, the dose of genetically engineered cells comprises from at or about 1×10⁵ to at or about 5×10⁹ total CAR-expressing (CAR+) T cells, from at or about 1×10⁵ to at or about 2.5×10⁸ total CAR-expressing T cells, from at or about 1×10⁵ to at or about 1×10⁸ total CAR-expressing T cells, from at or about 1×10⁵ to at or about 5×10⁷ total CAR-expressing T cells, from at or about 1×10⁵ to at or about 2.5×10⁷ total CAR-expressing T cells, from at or about 1×10⁵ to at or about 1×10⁷ total CAR-expressing T cells, from at or about 1×10⁵ to at or about 5×10⁶ total CAR-expressing T cells, from at or about 1×10⁵ to at or about 2.5×10⁶ total CAR-expressing T cells, from at or about 1×10⁵ to at or about 1×10⁶ total CAR-expressing T cells, from at or about 1×10⁶ to at or about 5×10⁸ total CAR-expressing T cells, from at or about 1×10⁶ to at or about 2.5×108 total CAR-expressing T cells, from at or about 1×10⁶ to at or about 1×10⁸ total CAR-expressing T cells, from at or about 1×10⁶ to at or about 5×10⁷ total CAR-expressing T cells, from at or about 1×10⁶ to at or about 2.5×10⁷ total CAR-expressing T cells, from at or about 1×10⁶ to at or about 1×10⁷ total CAR-expressing T cells, from at or about 1×10⁶ to at or about 5×10⁶ total CAR-expressing T cells, from at or about 1×10⁶ to at or about 2.5×10⁶ total CAR-expressing T cells, from at or about 2.5×10⁶ to at or about 5×10⁸ total CAR-expressing T cells, from at or about 2.5×10⁶ to at or about 2.5×10⁸ total CAR-expressing T cells, from at or about 2.5×10⁶ to at or about 1×10⁸ total CAR-expressing T cells, from at or about 2.5×10⁶ to at or about 5×10⁷ total CAR-expressing T cells, from at or about 2.5×10⁶ to at or about 2.5×10⁷ total CAR-expressing T cells, from at or about 2.5×10⁶ to at or about 1×10⁷ total CAR-expressing T cells, from at or about 2.5×10⁶ to at or about 5×10⁶ total CAR-expressing T cells, from at or about 5×10⁶ to at or about 5×10⁸ total CAR-expressing T cells, from at or about 5×10⁶ to at or about 2.5×10⁸ total CAR-expressing T cells, from at or about 5×10⁶ to at or about 1×10⁸ total CAR-expressing T cells, from at or about 5×10⁶ to at or about 5×10⁷ total CAR-expressing T cells, from at or about 5×10⁶ to at or about 2.5×10⁷ total CAR-expressing T cells, from at or about 5×10⁶ to at or about 1×10⁷ total CAR-expressing T cells, from at or about 1×10⁷ to at or about 5×10⁸ total CAR-expressing T cells, from at or about 1×10⁷ to at or about 2.5×10⁸ total CAR-expressing T cells, from at or about 1×10⁷ to at or about 1×10⁸ total CAR-expressing T cells, from at or about 1×10⁷ to at or about 5×10⁷ total CAR-expressing T cells, from at or about 1×10⁷ to at or about 2.5×10⁷ total CAR-expressing T cells, from at or about 2.5×10⁷ to at or about 5×10⁸ total CAR-expressing T cells, from at or about 2.5×10⁷ to at or about 2.5×10⁸ total CAR-expressing T cells, from at or about 2.5×10⁷ to at or about 1×10⁸ total CAR-expressing T cells, from at or about 2.5×10⁷ to at or about 5×10⁷ total CAR-expressing T cells, from at or about 5×10⁷ to at or about 5×10⁸ total CAR-expressing T cells, from at or about 5×10⁷ to at or about 2.5×10⁸ total CAR-expressing T cells, from at or about 5×10⁷ to at or about 1×10⁸ total CAR-expressing T cells, from at or about 1×10⁸ to at or about 5×10⁹ total CAR-expressing T cells, from at or about 1×10⁸ to at or about 2.5×10⁸ total CAR-expressing T cells, from at or about or 2.5×10⁸ to at or about 5×10⁹ total CAR-expressing T cells. In some embodiments, the dose of genetically engineered cells comprises from or from about 2.5×10⁷ to at or about 1.5×10⁸ total CAR-expressing T cells, such as from or from about 5×10⁷ to or to about 1×10⁸ total CAR-expressing T cells.

In some embodiments, the dose of genetically engineered cells comprises at least at or about 1×10⁵ CAR-expressing cells, at least at or about 2.5×10⁵ CAR-expressing cells, at least at or about 5×10⁵ CAR-expressing cells, at least at or about 1×10⁶ CAR-expressing cells, at least at or about 2.5×10⁶ CAR-expressing cells, at least at or about 5×10⁶ CAR-expressing cells, at least at or about 1×10⁷ CAR-expressing cells, at least at or about 2.5×10⁷ CAR-expressing cells, at least at or about 5×10⁷ CAR-expressing cells, at least at or about 1×10⁸ CAR-expressing cells, at least at or about 1.5×10⁸ CAR-expressing cells, at least about 5×10⁶ CAR-expressing cells, at least or at least about 1×10⁷ CAR-expressing cells, at least or at least about 2.5×10⁷ CAR-expressing cells, at least or at least about 5×10⁷ CAR-expressing cells, at least or at least about 1×10⁸ CAR-expressing cells, at least or at least about 2.5×10⁸ CAR-expressing cells, or at least or at least about 5×10⁸ CAR-expressing cells.

In some embodiments, the cell therapy comprises administration of a dose comprising a number of cell from or from about 1×10⁵ to or to about 5×10⁸ total recombinant receptor-expressing cells, total T cells, or total peripheral blood mononuclear cells (PBMCs), from or from about 5×10⁵ to or to about 1×10⁷ total recombinant receptor-expressing cells, total T cells, or total peripheral blood mononuclear cells (PBMCs) or from or from about 1×10⁶ to or to about 1×10⁷ total recombinant receptor-expressing cells, total T cells, or total peripheral blood mononuclear cells (PBMCs), each inclusive. In some embodiments, the cell therapy comprises administration of a dose of cells comprising a number of cells at least or at least about 1×10⁵ total recombinant receptor-expressing cells, total T cells, or total peripheral blood mononuclear cells (PBMCs), such at least or at least 1×10⁶, at least or at least about 1×10⁷, at least or at least about 1×10⁸ of such cells. In some embodiments, the number is with reference to the total number of CD3-expressing or CD8-expressing, in some cases also recombinant receptor-expressing (e.g., CAR-expressing) cells. In some embodiments, the cell therapy comprises administration of a dose comprising a number of cell from or from about 1×10⁵ to or to about 5×10⁸ CD3-expressing or CD8-expressing total T cells or CD3-expressing or CD8-expressing recombinant receptor-expressing cells, from or from about 5×10⁵ to or to about 1×10⁷ CD3-expressing or CD8-expressing total T cells or CD3-expressing or CD8-expressing recombinant receptor-expressing cells, or from or from about 1×10⁶ to or to about 1×10⁷ CD3-expressing or CD8-expressing total T cells or CD3-expressing or CD8-expressing recombinant receptor-expressing cells, each inclusive. In some embodiments, the cell therapy comprises administration of a dose comprising a number of cell from or from about 1×10⁵ to or to about 5×10⁸ total CD3-expressing/CAR-expressing or CD8-expressing/CAR-expressing cells, from or from about 5×10⁵ to or to about 1×10⁷ total CD3-expressing/CAR-expressing or CD8-expressing/CAR-expressing cells, or from or from about 1×10⁶ to or to about 1×10⁷ total CD3-expressing/CAR-expressing or CD8-expressing/CAR-expressing cells, each inclusive.

In some embodiments, the T cells of the dose include CD4+ T cells, CD8+ T cells or CD4+ and CD8+ T cells.

In some embodiments, for example, where the subject is human, the CD8+ T cells of the dose, including in a dose including CD4+ and CD8+ T cells, includes between at or about 1×10⁶ and at or about 5×10⁸ total recombinant receptor (e.g., CAR)-expressing CD8+ cells, e.g., in the range of from at or about 5×10⁶ to at or about 1×10⁸ such cells, such as 1×10⁷, 2.5×10⁷, 5×10⁷, 7.5×10⁷, 1×10⁸, 1.5×10⁸, or 5×10⁸ total such cells, or the range between any two of the foregoing values. In some embodiments, the patient is administered multiple doses, and each of the doses or the total dose can be within any of the foregoing values. In some embodiments, the dose of cells comprises the administration of from or from about 1×10⁷ to or to about 0.75×10⁸ total recombinant receptor-expressing CD8+ T cells, from or from about 1×10⁷ to or to about 5×10⁷ total recombinant receptor-expressing CD8+ T cells, from or from about 1×10⁷ to or to about 0.25×10⁸ total recombinant receptor-expressing CD8+ T cells, each inclusive. In some embodiments, the dose of cells comprises the administration of at or about 1×10⁷, 2.5×10⁷, 5×10⁷, 7.5×10⁷, 1×10⁸, 1.5×10⁸, 2.5×10⁸, or 5×10⁸ total recombinant receptor-expressing CD8+ T cells.

In some embodiments, the dose of cells, e.g., recombinant receptor-expressing T cells, is administered to the subject as a single dose or is administered only one time within a period of two weeks, one month, three months, six months, 1 year or more.

In the context of adoptive cell therapy, administration of a given “dose” encompasses administration of the given amount or number of cells as a single composition and/or single uninterrupted administration, e.g., as a single injection or continuous infusion, and also encompasses administration of the given amount or number of cells as a split dose or as a plurality of compositions, provided in multiple individual compositions or infusions, over a specified period of time, such as over no more than 3 days. Thus, in some contexts, the dose is a single or continuous administration of the specified number of cells, given or initiated at a single point in time. In some contexts, however, the dose is administered in multiple injections or infusions over a period of no more than three days, such as once a day for three days or for two days or by multiple infusions over a single day period.

Thus, in some aspects, the cells of the dose are administered in a single pharmaceutical composition. In some embodiments, the cells of the dose are administered in a plurality of compositions, collectively containing the cells of the dose.

In some embodiments, the term “split dose” refers to a dose that is split so that it is administered over more than one day. This type of dosing is encompassed by the present methods and is considered to be a single dose.

Thus, the dose of cells may be administered as a split dose, e.g., a split dose administered over time. For example, in some embodiments, the dose may be administered to the subject over 2 days or over 3 days. Exemplary methods for split dosing include administering 25% of the dose on the first day and administering the remaining 75% of the dose on the second day. In other embodiments, 33% of the dose may be administered on the first day and the remaining 67% administered on the second day. In some aspects, 10% of the dose is administered on the first day, 30% of the dose is administered on the second day, and 60% of the dose is administered on the third day. In some embodiments, the split dose is not spread over more than 3 days.

In some embodiments, cells of the dose may be administered by administration of a plurality of compositions or solutions, such as a first and a second, optionally more, each containing some cells of the dose. In some aspects, the plurality of compositions, each containing a different population and/or sub-types of cells, are administered separately or independently, optionally within a certain period of time. For example, the populations or sub-types of cells can include CD8⁺ and CD4⁺ T cells, respectively, and/or CD8⁺- and CD4⁺-enriched populations, respectively, e.g., CD4⁺ and/or CD8⁺ T cells each individually including cells genetically engineered to express the recombinant receptor. In some embodiments, the administration of the dose comprises administration of a first composition comprising a dose of CD8⁺ T cells or a dose of CD4⁺ T cells and administration of a second composition comprising the other of the dose of CD4⁺ T cells and the CD8⁺ T cells.

In some embodiments, the administration of the composition or dose, e.g., administration of the plurality of cell compositions, involves administration of the cell compositions separately. In some aspects, the separate administrations are carried out simultaneously, or sequentially, in any order. In some embodiments, the dose comprises a first composition and a second composition, and the first composition and second composition are administered from at or about 0 to at or about 12 hours apart, from at or about 0 to at or about 6 hours apart or from at or about 0 to at or about 2 hours apart. In some embodiments, the initiation of administration of the first composition and the initiation of administration of the second composition are carried out no more than at or about 2 hours, no more than at or about 1 hour, or no more than at or about 30 minutes apart, no more than at or about 15 minutes, no more than at or about 10 minutes or no more than at or about 5 minutes apart. In some embodiments, the initiation and/or completion of administration of the first composition and the completion and/or initiation of administration of the second composition are carried out no more than at or about 2 hours, no more than at or about 1 hour, or no more than at or about 30 minutes apart, no more than at or about 15 minutes, no more than at or about 10 minutes or no more than at or about 5 minutes apart.

In some composition, the first composition, e.g., first composition of the dose, comprises CD4⁺ T cells. In some composition, the first composition, e.g., first composition of the dose, comprises CD8⁺ T cells. In some embodiments, the first composition is administered prior to the second composition.

In some embodiments, the dose or composition of cells includes a defined or target ratio of CD4⁺ cells expressing a recombinant receptor to CD8⁺ cells expressing a recombinant receptor and/or of CD4⁺ cells to CD8⁺ cells, which ratio optionally is approximately 1:1 or is between approximately 1:3 and approximately 3:1, such as approximately 1:1. In some aspects, the administration of a composition or dose with the target or desired ratio of different cell populations (such as CD4⁺:CD8⁺ ratio or CAR⁺CD4⁺:CAR⁺CD8⁺ ratio, e.g., 1:1) involves the administration of a cell composition containing one of the populations and then administration of a separate cell composition comprising the other of the populations, where the administration is at or approximately at the target or desired ratio. In some aspects, administration of a dose or composition of cells at a defined ratio leads to improved expansion, persistence and/or antitumor activity of the T cell therapy.

In some embodiments, the subject receives multiple doses, e.g., two or more doses or multiple consecutive doses, of the cells. In some embodiments, two doses are administered to a subject. In some embodiments, the subject receives the consecutive dose, e.g., second dose, is administered approximately 4, 5, 6, 7, 8, 9, 10, 11, 12, 13, 14, 15, 16, 17, 18, 19, 20 or 21 days after the first dose. In some embodiments, multiple consecutive doses are administered following the first dose, such that an additional dose or doses are administered following administration of the consecutive dose. In some aspects, the number of cells administered to the subject in the additional dose is the same as or similar to the first dose and/or consecutive dose. In some embodiments, the additional dose or doses are larger than prior doses.

In some aspects, the size of the first and/or consecutive dose is determined based on one or more criteria such as response of the subject to prior treatment, e.g., chemotherapy, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

In some aspects, the time between the administration of the first dose and the administration of the consecutive dose is about 9 to about 35 days, about 14 to about 28 days, or 15 to 27 days. In some embodiments, the administration of the consecutive dose is at a time point more than about 14 days after and less than about 28 days after the administration of the first dose. In some aspects, the time between the first and consecutive dose is about 21 days. In some embodiments, an additional dose or doses, e.g., consecutive doses, are administered following administration of the consecutive dose. In some aspects, the additional consecutive dose or doses are administered at least about 14 and less than about 28 days following administration of a prior dose. In some embodiments, the additional dose is administered less than about 14 days following the prior dose, for example, 4, 5, 6, 7, 8, 9, 10, 11, 12, or 13 days after the prior dose. In some embodiments, no dose is administered less than about 14 days following the prior dose and/or no dose is administered more than about 28 days after the prior dose.

In some embodiments, the dose of cells, e.g., recombinant receptor-expressing cells, comprises two doses (e.g., a double dose), comprising a first dose of the T cells and a consecutive dose of the T cells, wherein one or both of the first dose and the second dose comprises administration of the split dose of T cells.

In some embodiments, the dose of cells is generally large enough to be effective in reducing disease burden.

In some embodiments, the cells are administered at a desired dosage, which in some aspects includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4⁺ to CD8⁺ ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In some embodiments, the populations or sub-types of cells, such as CD8⁺ and CD4⁺ T cells, are administered at or within a tolerated difference of a desired dose of total cells, such as a desired dose of T cells. In some aspects, the desired dose is a desired number of cells or a desired number of cells per unit of body weight of the subject to whom the cells are administered, e.g., cells/kg. In some aspects, the desired dose is at or above a minimum number of cells or minimum number of cells per unit of body weight. In some aspects, among the total cells, administered at the desired dose, the individual populations or sub-types are present at or near a desired output ratio (such as CD4⁺ to CD8⁺ ratio), e.g., within a certain tolerated difference or error of such a ratio.

In some embodiments, the cells are administered at or within a tolerated difference of a desired dose of one or more of the individual populations or sub-types of cells, such as a desired dose of CD4⁺ cells and/or a desired dose of CD8⁺ cells. In some aspects, the desired dose is a desired number of cells of the sub-type or population, or a desired number of such cells per unit of body weight of the subject to whom the cells are administered, e.g., cells/kg. In some aspects, the desired dose is at or above a minimum number of cells of the population or sub-type, or minimum number of cells of the population or sub-type per unit of body weight.

Thus, in some embodiments, the dosage is based on a desired fixed dose of total cells and a desired ratio, and/or based on a desired fixed dose of one or more, e.g., each, of the individual sub-types or sub-populations. Thus, in some embodiments, the dosage is based on a desired fixed or minimum dose of T cells and a desired ratio of CD4⁺ to CD8⁺ cells, and/or is based on a desired fixed or minimum dose of CD4⁺ and/or CD8⁺ cells.

In some embodiments, the cells are administered at or within a tolerated range of a desired output ratio of multiple cell populations or sub-types, such as CD4⁺ and CD8⁺ cells or sub-types. In some aspects, the desired ratio can be a specific ratio or can be a range of ratios. for example, in some embodiments, the desired ratio (e.g., ratio of CD4⁺ to CD8⁺ cells) is between at or about 5:1 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1, such as at or about 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9: 1:2, 1:2.5, 1:3, 1:3.5, 1:4, 1:4.5, or 1:5. In some aspects, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges.

In particular embodiments, the numbers and/or concentrations of cells refer to the number of recombinant receptor (e.g., CAR)-expressing cells. In other embodiments, the numbers and/or concentrations of cells refer to the number or concentration of all cells, T cells, or peripheral blood mononuclear cells (PBMCs) administered.

In some aspects, the size of the dose is determined based on one or more criteria such as response of the subject to prior treatment, e.g., chemotherapy, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

In some embodiments, the methods also include administering one or more additional doses of cells expressing a chimeric antigen receptor (CAR) and/or lymphodepleting therapy, and/or one or more steps of the methods are repeated. In some embodiments, the one or more additional dose is the same as the initial dose. In some embodiments, the one or more additional dose is different from the initial dose, e.g., higher, such as 2-fold, 3-fold, 4-fold, 5-fold, 6-fold, 7-fold, 8-fold, 9-fold or 10-fold or more higher than the initial dose, or lower, such as e.g., higher, such as 2-fold, 3-fold, 4-fold, 5-fold, 6-fold, 7-fold, 8-fold, 9-fold or 10-fold or more lower than the initial dose. In some embodiments, administration of one or more additional doses is determined based on response of the subject to the initial treatment or any prior treatment, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered. In some embodiments, administration of one or more additional doses is determined based on the clinical responses determined according the methods provided herein.

### V. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”

As used herein, recitation that nucleotides or amino acid positions “correspond to” nucleotides or amino acid positions in a disclosed sequence, such as set forth in the Sequence listing, refers to nucleotides or amino acid positions identified upon alignment with the disclosed sequence to maximize identity using a standard alignment algorithm, such as the GAP algorithm. By aligning the sequences, one skilled in the art can identify corresponding residues, for example, using conserved and identical amino acid residues as guides. In general, to identify corresponding positions, the sequences of amino acids are aligned so that the highest order match is obtained (see, e.g., Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carrillo et al. (1988) SIAM J Applied Math 48: 1073).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.” Among the vectors are viral vectors, such as retroviral, e.g., gammaretroviral and lentiviral vectors.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

As used herein, a statement that a cell or population of cells is “positive” for a particular marker refers to the detectable presence on or in the cell of a particular marker, typically a surface marker. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker, typically a surface marker. When referring to a surface marker, the term refers to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

As used herein, “percent (%) amino acid sequence identity” and “percent identity” when used with respect to an amino acid sequence (reference polypeptide sequence) is defined as the percentage of amino acid residues in a candidate sequence (e.g., the subject antibody or fragment) that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

An amino acid substitution may include replacement of one amino acid in a polypeptide with another amino acid. The substitution may be a conservative amino acid substitution or a non-conservative amino acid substitution. Amino acid substitutions may be introduced into a binding molecule, e.g., antibody, of interest and the products screened for a desired activity, e.g., retained/improved antigen binding, decreased immunogenicity, or improved ADCC or CDC.

Amino acids generally can be grouped according to the following common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

In some embodiments, conservative substitutions can involve the exchange of a member of one of these classes for another member of the same class. In some embodiments, non-conservative amino acid substitutions can involve exchanging a member of one of these classes for another class.

As used herein, a composition refers to any mixture of two or more products, substances, or compounds, including cells. It may be a solution, a suspension, liquid, powder, a paste, aqueous, non-aqueous or any combination thereof.

As used herein, a “subject” is a mammal, such as a human or other animal, and typically is human.

### VI. Exemplary Embodiments

Among the provided embodiments are:
1. A method of predicting whether a subject will exhibit a clinical response to a T cell therapy, comprising:
(a) obtaining, for a subject having a disease or condition, a parameter of a marker or parameters of a combination of markers, wherein:
   (i) the parameter or parameters are obtained prior to the subject being administered a T cell therapy comprising T cells comprising a recombinant receptor that binds to an antigen associated with the disease or condition; and
   (ii) the marker or the combination of markers is selected from the (1) level in a blood sample of d-dimer, (2) level in a blood sample of fibrinogen, (3) level in a blood sample of lymphocytes, (4) level in a blood sample of monocytes, (5) ratio in a blood sample of monocytes to leukocytes, (6) level in a blood sample of red blood cells, (7) level in a blood sample of white blood cells, (8) age, (9) body mass index, (10) level in a blood sample of albumin, (11) level in a blood sample of alkaline phosphatase, (12) level in a blood sample of aspartate aminotransferase, (13) level in a blood sample of alanine aminotransferase, (14) level in a blood sample of direct bilirubin, (15) level in a blood sample of bilirubin, (16) level in a blood sample of creatinine, (17) creatinine clearance, (18) time since diagnosis, (19) number of prior therapies received, (20) time since prior autologous stem cell transplant, (21) time since prior corticosteroid therapy, (22) time since prior alkylating agent therapy, (23) time since prior topoisomerase inhibitor therapy, (24) time since prior proteasome inhibitor therapy, (25) percent in a blood sample of bone marrow plasma cells, (26) level in a blood sample of beta-2 microglobulin, (27) level in a blood sample of Immunoglobulin G, (28) level in a blood sample of lactate dehydrogenase, (29) ratio in a blood sample of kappa to lambda free light chain levels, (30) level in a blood sample of free light chain, (31) level in a blood sample of M-protein, (32) level in a blood sample of platelets, (33) level in a blood sample of sodium, and (34) level in a blood sample of soluble BCMA of the subject; and
(b) predicting if the subject is likely to exhibit a clinical response to administration of the T cell therapy for treatment of the disease or condition, wherein the subject is predicted as likely to exhibit the clinical response if:
   (i) the parameter or one or more of the parameters for markers (3), (6)-(13), (16), (17), (20), (22)-(24), (28), (29), (32), and (33) are higher than an associated threshold level; or
   (ii) the parameter or one or more of the parameters for markers (1), (2), (4), (5), (14), (15), (18), (19), (21), (25)-(27), (30), (31), and (34) are lower than an associated threshold level.
2. The method of embodiment 1, wherein the subject is predicted as likely to exhibit the clinical response if two or more, three or more, or four or more of any of the criteria of step (b)(i)-(b)(ii) are satisfied.
3. A method of predicting whether a subject will not exhibit a clinical response to a T cell therapy, comprising:
(a) obtaining, for a subject having a disease or condition, a parameter of a marker or parameters of a combination of markers, wherein:
   (i) the parameter or parameters are obtained prior to the subject being administered a T cell therapy comprising T cells comprising a recombinant receptor that binds to an antigen associated with the disease or condition; and
   (ii) the marker or the combination of markers is selected from the (1) level in a blood sample of d-dimer, (2) level in a blood sample of fibrinogen, (3) level in a blood sample of lymphocytes, (4) level in a blood sample of monocytes, (5) ratio in a blood sample of monocytes to leukocytes, (6) level in a blood sample of red blood cells, (7) level in a blood sample of white blood cells, (8) age, (9) body mass index, (10) level in a blood sample of albumin, (11) level in a blood sample of alkaline phosphatase, (12) level in a blood sample of aspartate aminotransferase, (13) level in a blood sample of alanine aminotransferase, (14) level in a blood sample of direct bilirubin, (15) level in a blood sample of bilirubin, (16) level in a blood sample of creatinine, (17) creatinine clearance, (18) time since diagnosis, (19) number of prior therapies received, (20) time since prior autologous stem cell transplant, (21) time since prior corticosteroid therapy, (22) time since prior alkylating agent therapy, (23) time since prior topoisomerase inhibitor therapy, (24) time since prior proteasome inhibitor therapy, (25) percent in a blood sample of bone marrow plasma cells, (26) level in a blood sample of beta-2 microglobulin, (27) level in a blood sample of Immunoglobulin G, (28) level in a blood sample of lactate dehydrogenase, (29) ratio in a blood sample of kappa to lambda free light chain levels, (30) level in a blood sample of free light chain, (31) level in a blood sample of M-protein, (32) level in a blood sample of platelets, (33) level in a blood sample of sodium, and (34) level in a blood sample of soluble BCMA of the subject; and
(b) predicting if the subject is likely to not exhibit a clinical response to administration of the T cell therapy for treatment of the disease or condition, wherein the subject is predicted as likely to not exhibit the clinical response if:
   (i) the parameter or one or more of the parameters for markers (3), (6)-(13), (16), (17), (20), (22)-(24), (28), (29), (32), and (33) are lower than an associated threshold level; or
   (ii) the parameter or one or more of the parameters for markers (1), (2), (4), (5), (14), (15), (18), (19), (21), (25)-(27), (30), (31), and (34) are higher than an associated threshold level.
4. The method of embodiment 3, wherein the subject is predicted as likely to not exhibit the clinical response if two or more, three or more, or four or more of any of the criteria of step (b)(i)-(b)(ii) are satisfied.
5. The method of any one of embodiments 1-4, wherein the marker is or the combination of markers comprises one or more subject immune profile markers that are selected from markers (1)-(7).
6. The method of any one of embodiments 1-5, wherein the marker is or the combination of markers comprises one or more subject fitness markers that are selected from markers (8)-(17).
7. The method of any one of embodiments 1-6, wherein the marker is or the combination of markers comprises one or more subject prior therapy markers that are selected from markers (18)-(24).
8. The method of any one of embodiments 1-7, wherein the marker is or the combination of markers comprises one or more subject tumor burden markers that are selected from markers (25)-(34).
9. The method of any one of embodiments 1-8, wherein:
the threshold level associated with marker (1) is between or between about 0.5 mg/L and 11 mg/L or between or between about 0.5 mg/L and 1.3 mg/L;
the threshold level associated with marker (2) is between or between about 2.2 g/L and 7.7 g/L or between or between about 4.2 g/L and 5.4 g/L;
the threshold level associated with marker (3) is between or between about 0.3×10⁹ cells/L and 1.0×10⁹ cells/L or between or between about 0.4×10⁹ cells/L and 0.7×10⁹ cells/L;
the threshold level associated with marker (4) is between or between about 0.2×10⁹ cells/L and 1.1×10⁹ cells/L or between or between about 0.4×10⁹ cells/L and 0.7×10⁹ cells/L;
the threshold level associated with marker (5) is between or between about 6.7 and 18 or between or between about 13 and 14;
the threshold level associated with marker (6) is between or between about 2.4×10¹² cells/L and 3.7×10¹² cells/L or between or between about 2.9×10¹² cells/L and 3.3×10¹² cells/L;
the threshold level associated with marker (7) is between or between about 2.1×10⁹ cells/L and 7.1×10⁹ cells/L or between or between about 2.9×10⁹ cells/L and 4.2×10⁹ cells/L;
the threshold level associated with marker (8) is between or between about 57 years and 66 years or between or between about 64 years and 66 years;
the threshold level associated with marker (9) is between or between about 22 kg/m² and 31 kg/m² or between or between about 23 kg/m² and 29 kg/m²;
the threshold level associated with marker (10) is between or between about 31 g/L and 41 g/L or between or between about 36 g/L and 40 g/L;
the threshold level associated with marker (11) is between or between about 28 IU/L and 134 IU/L or between or between about 54 IU/L and 64 IU/L;
the threshold level associated with marker (12) is between or between about 7.3 IU/L and 49 IU/L or between or between about 16 IU/L and 26 IU/L;
the threshold level associated with marker (13) is between or between about 8 IU/L and 31 IU/L or between or between about 13 IU/L and 29 IU/L;
the threshold level associated with marker (14) is between or between about 1.4 μM and 2.7 μM or between or between about 1.8 μM and 2.2 μM;
the threshold level associated with marker (15) is between or between about 3.4 μM and 23 μM or between or between about 9.4 μM and 9.6 μM;
the threshold level associated with marker (16) is between or between about 46 μM and 114 μM or between or between about 52 μM and 80 μM;
the threshold level associated with marker (17) is between or between about 0.8 mL/s and 2.0 mL/s or between or between about 1.9 mL/s and 2.0 mL/s;
the threshold level associated with marker (18) is between or between about 2.2 years and 10 years or between or between about 5.5 years and 8.3 years;
the threshold level associated with marker (19) is between or between about 4 and 11 or between or between about 4 and 5;
the threshold level associated with marker (20) is between or between about 26 days and 3205 days or between or between about 641 days and 2941 days;
the threshold level associated with marker (21) is between or between about 12 days and 2257 days or between or between about 42 days and 59 days;
the threshold level associated with marker (22) is between or between about 11 days and 493 days or between or between about 230 days and 244 days;
the threshold level associated with marker (23) is between or between about 87 days and 3356 days or between or between about 474 days and 676 days;
the threshold level associated with marker (24) is between or between about 11 days and 658 days or between or between about 51 days and 170 days;
the threshold level associated with marker (25) is between or between about 21% and 100% or between or between about 56% and 80%;
the threshold level associated with marker (26) is between or between about 2.7 mg/L and 7.7 mg/L or between or between about 3.2 mg/L and 4.6 mg/L;
the threshold level associated with marker (27) is between or between about 2.8 g/L and 75 g/L or between or between about 14 g/L and 35 g/L;
the threshold level associated with marker (28) is between or between about 150 IU/L and 319 IU/L or between or between about 181 IU/L and 319 IU/L;
the threshold level associated with marker (29) is between or between about 0.003 and 763 or between or between about 8.7 and 211;
the threshold level associated with marker (30) is between or between about 0.008 g/L and 12 g/L or between or between about 0.2 g/L and 1.0 g/L;
the threshold level associated with marker (31) is between or between about 4.3 g/L and 32 g/L or between or between about 5.3 g/L and 12 g/L;
the threshold level associated with marker (32) is between or between about 53×10⁹ cells/L and 212×10⁹ cells/L or between or between about 156×10⁹ cells/L and 181×10⁹ cells/L;
the threshold level associated with marker (33) is between or between about 132 mM and 141 mM or between or between about 136 mM and 138 mM; and/or
the threshold level associated with marker (34) is between or between about 35 ng/mL and 1300 ng/mL or between or between about 170 ng/mL and 654 ng/mL.
10. A method of predicting whether a subject will exhibit a clinical response to a T cell therapy, comprising:
(a) obtaining, for a subject having a disease or condition, a parameter of a marker or parameters of a combination of markers, wherein:
   (i) the parameter or parameters are obtained prior to the subject being administered a T cell therapy comprising T cells comprising a recombinant receptor that binds to an antigen associated with the disease or condition; and
   (ii) the marker or the combination of markers is selected from any of one or more subject immune profile markers, one or more subject fitness markers, one or more subject prior therapy markers, and one or more subject tumor burden markers of the subject; and
(b) predicting if the subject is likely to exhibit a clinical response to administration of the T cell therapy for treatment of the disease or condition, wherein the predicting comprises comparing the parameter or each of the parameters to an associated threshold level.
11. A method of predicting whether a subject will not exhibit a clinical response to a T cell therapy, comprising:
(a) obtaining, for a subject having a disease or condition, a parameter of a marker or parameters of a combination of markers, wherein:
   (i) the parameter or parameters are obtained prior to the subject being administered a T cell therapy comprising T cells comprising a recombinant receptor that binds to an antigen associated with the disease or condition; and
   (ii) the marker or the combination of markers is selected from any of one or more subject immune profile markers, one or more subject fitness markers, one or more subject prior therapy markers, and one or more subject tumor burden markers of the subject; and
(b) predicting if the subject is likely to not exhibit a clinical response to administration of the T cell therapy for treatment of the disease or condition, wherein the predicting comprises comparing the parameter or each of the parameters to an associated threshold level.
12. The method of embodiment 10 or embodiment 11, wherein the parameters of a combination of markers are obtained, and each of the parameters is compared to an associated threshold level.
13. The method of any one of embodiments 10-12, wherein the combination of markers comprises one or more subject immune profile markers.
14. The method of embodiment 13, wherein the one or more subject immune profile markers are selected from the (1) level in a blood sample of d-dimer, (2) level in a blood sample of fibrinogen, (3) level in a blood sample of lymphocytes, (4) level in a blood sample of monocytes, (5) ratio in a blood sample of monocytes to leukocytes, (6) level in a blood sample of red blood cells, and (7) level in a blood sample of white blood cells of the subject.
15. The method of embodiment 14, wherein the subject is predicted as likely to exhibit the clinical response if:
(i) the parameter or one or more of the parameters for markers (3), (6), and (7) are higher than an associated threshold level; or
(ii) the parameter or one or more of the parameters for markers (1), (2), (4), and (5) are lower than an associated threshold level.
16. The method of embodiment 14, wherein the subject is predicted as likely to not exhibit the clinical response if:
(i) the parameter or one or more of the parameters for markers (3), (6), and (7) are lower than an associated threshold level; or
(ii) the parameter or one or more of the parameters for markers (1), (2), (4), and (5) are higher than an associated threshold level.
17. The method of any one of embodiments 14-16, wherein:
the threshold level associated with marker (1) is between or between about 0.5 mg/L and 11 mg/L or between or between about 0.5 mg/L and 1.3 mg/L;
the threshold level associated with marker (2) is between or between about 2.2 g/L and 7.7 g/L or between or between about 4.2 g/L and 5.4 g/L;
the threshold level associated with marker (3) is between or between about 0.3×10⁹ cells/L and 1.0×10⁹ cells/L or between or between about 0.4×10⁹ cells/L and 0.7×10⁹ cells/L;
the threshold level associated with marker (4) is between or between about 0.2×10⁹ cells/L and 1.1×10⁹ cells/L or between or between about 0.4×10⁹ cells/L and 0.7×10⁹ cells/L;
the threshold level associated with marker (5) is between or between about 6.7 and 18 or between or between about 13 and 14;
the threshold level associated with marker (6) is between or between about 2.4×10¹² cells/L and 3.7×10¹² cells/L or between or between about 2.9×10¹² cells/L and 3.3×10¹² cells/L; and/or
the threshold level associated with marker (7) is between or between about 2.1×10⁹ cells/L and 7.1×10⁹ cells/L or between or between about 2.9×10⁹ cells/L and 4.2×10⁹ cells/L.
18. The method of any one of embodiments 10-17, wherein the combination of markers comprises one or more subject fitness markers.
19. The method of embodiment 18, wherein the one or more subject fitness markers are selected from the (8) age, (9) body mass index, (10) level in a blood sample of albumin, (11) level in a blood sample of alkaline phosphatase, (12) level in a blood sample of aspartate aminotransferase, (13) level in a blood sample of alanine aminotransferase, (14) level in a blood sample of direct bilirubin, (15) level in a blood sample of bilirubin, (16) level in a blood sample of creatinine, and (17) creatinine clearance of the subject.
20. The method of embodiment 19, wherein the subject is predicted as likely to exhibit the clinical response if:
(i) the parameter or one or more of the parameters for markers (8)-(13), (16), and (17) are higher than an associated threshold level; or
(ii) the parameter or one or more of the parameters for markers (14) and (15) are lower than an associated threshold level.
21. The method of embodiment 19, wherein the subject is predicted as likely to not exhibit the clinical response if:
(i) the parameter or one or more of the parameters for markers (8)-(13), (16), and (17) are lower than an associated threshold level; or
(ii) the parameter or one or more of the parameters for markers (14) and (15) are higher than an associated threshold level.
22. The method of any one of embodiments 19-21, wherein:
the threshold level associated with marker (8) is between or between about 57 years and 66 years or between or between about 64 years and 66 years;
the threshold level associated with marker (9) is between or between about 22 kg/m² and 31 kg/m² or between or between about 23 kg/m² and 29 kg/m²;
the threshold level associated with marker (10) is between or between about 31 g/L and 41 g/L or between or between about 36 g/L and 40 g/L;
the threshold level associated with marker (11) is between or between about 28 IU/L and 134 IU/L or between or between about 54 IU/L and 64 IU/L;
the threshold level associated with marker (12) is between or between about 7.3 IU/L and 49 IU/L or between or between about 16 IU/L and 26 IU/L;
the threshold level associated with marker (13) is between or between about 8 IU/L and 31 IU/L or between or between about 13 IU/L and 29 IU/L;
the threshold level associated with marker (14) is between or between about 1.4 μM and 2.7 μM or between or between about 1.8 μM and 2.2 μM;
the threshold level associated with marker (15) is between or between about 3.4 μM and 23 μM or between or between about 9.4 μM and 9.6 μM;
the threshold level associated with marker (16) is between or between about 46 μM and 114 μM or between or between about 52 μM and 80 μM; and/or
the threshold level associated with marker (17) is between or between about 0.8 mL/s and 2.0 mL/s or between or between about 1.9 mL/s and 2.0 mL/s.
23. The method of any one of embodiments 10-22, wherein the combination of markers comprises one or more subject prior therapy markers.
24. The method of embodiment 23, wherein the one or more subject prior therapy markers are selected from the (18) time since diagnosis, (19) number of prior therapies received, (20) time since prior autologous stem cell transplant, (21) time since prior corticosteroid therapy, (22) time since prior alkylating agent therapy, (23) time since prior topoisomerase inhibitor therapy, and (24) time since prior proteasome inhibitor therapy for the subject.
25. The method of embodiment 24, wherein the subject is predicted as likely to exhibit the clinical response if:
(i) the parameter or one or more of the parameters for markers (20) and (22)-(24) are higher than an associated threshold level; or
(ii) the parameter or one or more of the parameters for markers (18), (19), and (21) are lower than an associated threshold level.
26. The method of embodiment 24, wherein the subject is predicted as likely to not exhibit the clinical response if:
(i) the parameter or one or more of the parameters for markers (20) and (22)-(24) are lower than an associated threshold level; or
(ii) the parameter or one or more of the parameters for markers (18), (19), and (21) are higher than an associated threshold level.
27. The method of any one of embodiments 24-26, wherein:
the threshold level associated with marker (18) is between or between about 2.2 years and 10 years or between or between about 5.5 years and 8.3 years;
the threshold level associated with marker (19) is between or between about 4 and 11 or between or between about 4 and 5;
the threshold level associated with marker (20) is between or between about 26 days and 3205 days or between or between about 641 days and 2941 days;
the threshold level associated with marker (21) is between or between about 12 days and 2257 days or between or between about 42 days and 59 days;
the threshold level associated with marker (22) is between or between about 11 days and 493 days or between or between about 230 days and 244 days;
the threshold level associated with marker (23) is between or between about 87 days and 3356 days or between or between about 474 days and 676 days; and/or
the threshold level associated with marker (24) is between or between about 11 days and 658 days or between or between about 51 days and 170 days.
28. The method of any one of embodiments 10-27, wherein the combination of markers comprises one or more subject tumor burden markers.
29. The method of embodiment 28, wherein the one or more subject tumor burden markers are selected from the (25) percent in a blood sample of bone marrow plasma cells, (26) level in a blood sample of beta-2 microglobulin, (27) level in a blood sample of Immunoglobulin G, (28) level in a blood sample of lactate dehydrogenase, (29) ratio in a blood sample of kappa to lambda free light chain levels, (30) level in a blood sample of free light chain, (31) level in a blood sample of M-protein, (32) level in a blood sample of platelets, (33) level in a blood sample of sodium, and (34) level in a blood sample of soluble BCMA of the subject.
30. The method of embodiment 29, wherein the subject is predicted as likely to exhibit the clinical response if:
(i) the parameter or one or more of the parameters for markers (28), (29), (32), and (33) are higher than an associated threshold level; or
(ii) the parameter or one or more of the parameters for markers (25)-(27), (30), (31), and (34) are lower than an associated threshold level.
31. The method of embodiment 29, wherein the subject is predicted as likely to not exhibit the clinical response if:
(i) the parameter or one or more of the parameters for markers (28), (29), (32), and (33) are lower than an associated threshold level; or
(ii) the parameter or one or more of the parameters for markers (25)-(27), (30), (31), and (34) are higher than an associated threshold level.
32. The method of any one of embodiments 29-31, wherein:
the threshold level associated with marker (25) is between or between about 21% and 100% or between or between about 56% and 80%;
the threshold level associated with marker (26) is between or between about 2.7 mg/L and 7.7 mg/L or between or between about 3.2 mg/L and 4.6 mg/L;
the threshold level associated with marker (27) is between or between about 2.8 g/L and 75 g/L or between or between about 14 g/L and 35 g/L;
the threshold level associated with marker (28) is between or between about 150 IU/L and 319 IU/L or between or between about 181 IU/L and 319 IU/L;
the threshold level associated with marker (29) is between or between about 0.003 and 763 or between or between about 8.7 and 211;
the threshold level associated with marker (30) is between or between about 0.008 g/L and 12 g/L or between or between about 0.2 g/L and 1.0 g/L;
the threshold level associated with marker (31) is between or between about 4.3 g/L and 32 g/L or between or between about 5.3 g/L and 12 g/L;
the threshold level associated with marker (32) is between or between about 53×10⁹ cells/L and 212×10⁹ cells/L or between or between about 156×10⁹ cells/L and 181×10⁹ cells/L;
the threshold level associated with marker (33) is between or between about 132 mM and 141 mM or between or between about 136 mM and 138 mM; and/or
the threshold level associated with marker (34) is between or between about 35 ng/mL and 1300 ng/mL or between or between about 170 ng/mL and 654 ng/mL.
33. The method of any one of embodiments 1-32, wherein the combination of markers comprises the (3) level in a blood sample of lymphocytes, (22) time since prior alkylating agent therapy, and (26) level in a blood sample of beta-2 microglobulin of the subject.
34. The method of embodiment 33, wherein the subject is predicted as likely to exhibit the clinical response if:
(i) the parameter for marker (3) is higher than an associated threshold level;
(ii) the parameter for marker (22) is higher than an associated threshold level; or
(iii) the parameter for marker (26) is lower than an associated threshold level.
35. The method of embodiment 33 or embodiment 34, wherein the subject is predicted as likely to exhibit the clinical response if:
(i) the parameter for marker (3) is higher than an associated threshold level;
(ii) the parameter for marker (22) is higher than an associated threshold level; and
(iii) the parameter for marker (26) is lower than an associated threshold level.
36. The method of embodiment 33, wherein the subject is predicted as likely to not exhibit the clinical response if:
(i) the parameter for marker (3) is lower than an associated threshold level;
(ii) the parameter for marker (22) is lower than an associated threshold level; or
(iii) the parameter for marker (26) is higher than an associated threshold level.
37. The method of embodiment 33 or embodiment 36, wherein the subject is predicted as likely to not exhibit the clinical response if:
(i) the parameter for marker (3) is lower than an associated threshold level;
(ii) the parameter for marker (22) is lower than an associated threshold level; and
(iii) the parameter for marker (26) is higher than an associated threshold level.
38. The method of any one of embodiments 33-37, wherein:
the threshold level associated with marker (3) is between or between about 0.3×10⁹ cells/L and 1.0×10⁹ cells/L or between or between about 0.4×10⁹ cells/L and 0.7×10⁹ cells/L;
the threshold level associated with marker (22) is between or between about 11 days and 493 days or between or between about 230 days and 244 days; and/or
the threshold level associated with marker (26) is between or between about 2.7 mg/L and 7.7 mg/L or between or between about 3.2 mg/L and 4.6 mg/L.
39. The method of any one of embodiments 1-38, wherein the combination of markers comprise the (5) ratio in a blood sample of monocytes to leukocytes, (24) time since prior proteasome inhibitor therapy, (28) level in a blood sample of lactate dehydrogenase, and (31) level in a blood sample of M-protein of the subject.
40. The method of embodiment 39, wherein the subject is predicted as likely to exhibit the clinical response if
(i) the parameter for marker (5) is lower than an associated threshold level;
(ii) the parameter for marker (24) is higher than an associated threshold level;
(iii) the parameter for marker (28) is higher than an associated threshold level; or
(iv) the parameter for marker (31) is lower than an associated threshold level.
41. The method of embodiment 39 or embodiment 40, wherein the subject is predicted as likely to exhibit the clinical response if
(i) the parameter for marker (5) is lower than an associated threshold level;
(ii) the parameter for marker (24) is higher than an associated threshold level;
(iii) the parameter for marker (28) is higher than an associated threshold level; and
(iv) the parameter for marker (31) is lower than an associated threshold level.
42. The method of embodiment 39, wherein the subject is predicted as likely to not exhibit the clinical response if
(i) the parameter for marker (5) is higher than an associated threshold level;
(ii) the parameter for marker (24) is lower than an associated threshold level;
(iii) the parameter for marker (28) is lower than an associated threshold level; or
(iv) the parameter for marker (31) is higher than an associated threshold level.
43. The method of embodiment 39 or embodiment 42, wherein the subject is predicted as likely to not exhibit the clinical response if
(i) the parameter for marker (5) is higher than an associated threshold level;
(ii) the parameter for marker (24) is lower than an associated threshold level;
(iii) the parameter for marker (28) is lower than an associated threshold level; and
(iv) the parameter for marker (31) is higher than an associated threshold level.
44. The method of any one of embodiments 39-43, wherein:
the threshold level associated with marker (5) is between or between about 6.7 and 18 or between or between about 13 and 14;
the threshold level associated with marker (24) is between or between about 11 days and 658 days or between or between about 51 days and 170 days;
the threshold level associated with marker (28) is between or between about 150 IU/L and 319 IU/L or between or between about 181 IU/L and 319 IU/L; and/or
the threshold level associated with marker (31) is between or between about 4.3 g/L and 32 g/L or between or between about 5.3 g/L and 12 g/L.
45. A method of predicting whether a subject will exhibit a clinical response to a T cell therapy, comprising:
(a) obtaining, for a subject having a disease or condition, a parameter of a marker or parameters of a combination of markers, wherein:
   (i) the parameter or parameters are obtained prior to the subject being administered a T cell therapy comprising T cells comprising a recombinant receptor that binds to an antigen associated with the disease or condition; and
   (ii) the marker or the combination of markers is selected from the (1) level in a blood sample of d-dimer, (2) level in a blood sample of fibrinogen, (3) level in a blood sample of lymphocytes, (4) level in a blood sample of monocytes, (5) ratio in a blood sample of monocytes to leukocytes, (6) level in a blood sample of red blood cells, (7) level in a blood sample of white blood cells, (8) age, (9) body mass index, (10) level in a blood sample of albumin, (11) level in a blood sample of alkaline phosphatase, (12) level in a blood sample of aspartate aminotransferase, (13) level in a blood sample of alanine aminotransferase, (14) level in a blood sample of direct bilirubin, (15) level in a blood sample of bilirubin, (16) level in a blood sample of creatinine, (17) creatinine clearance, (18) time since diagnosis, (19) number of prior therapies received, (20) time since prior autologous stem cell transplant, (21) time since prior corticosteroid therapy, (22) time since prior alkylating agent therapy, (23) time since prior topoisomerase inhibitor therapy, (24) time since prior proteasome inhibitor therapy, (25) percent in a blood sample of bone marrow plasma cells, (26) level in a blood sample of beta-2 microglobulin, (27) level in a blood sample of Immunoglobulin G, (28) level in a blood sample of lactate dehydrogenase, (29) ratio in a blood sample of kappa to lambda free light chain levels, (30) level in a blood sample of free light chain, (31) level in a blood sample of M-protein, (32) level in a blood sample of platelets, (33) level in a blood sample of sodium, and (34) level in a blood sample of soluble BCMA of the subject; and
(b) predicting if the subject is likely to exhibit a clinical response to administration of the T cell therapy for treatment of the disease or condition based on one or more outputs of a process configured to predict, based on the marker or combination of markers, if the subject is likely to exhibit the clinical response, wherein the predicting comprises providing the parameter or parameters as input to the process.
46. A method of predicting whether a subject will not exhibit a clinical response to a T cell therapy, comprising:
(a) obtaining, for a subject having a disease or condition, a parameter of a marker or parameters of a combination of markers, wherein:
   (i) the parameter or parameters are obtained prior to the subject being administered a T cell therapy comprising T cells comprising a recombinant receptor that binds to an antigen associated with the disease or condition; and
   (ii) the marker or the combination of markers is selected from the (1) level in a blood sample of d-dimer, (2) level in a blood sample of fibrinogen, (3) level in a blood sample of lymphocytes, (4) level in a blood sample of monocytes, (5) ratio in a blood sample of monocytes to leukocytes, (6) level in a blood sample of red blood cells, (7) level in a blood sample of white blood cells, (8) age, (9) body mass index, (10) level in a blood sample of albumin, (11) level in a blood sample of alkaline phosphatase, (12) level in a blood sample of aspartate aminotransferase, (13) level in a blood sample of alanine aminotransferase, (14) level in a blood sample of direct bilirubin, (15) level in a blood sample of bilirubin, (16) level in a blood sample of creatinine, (17) creatinine clearance, (18) time since diagnosis, (19) number of prior therapies received, (20) time since prior autologous stem cell transplant, (21) time since prior corticosteroid therapy, (22) time since prior alkylating agent therapy, (23) time since prior topoisomerase inhibitor therapy, (24) time since prior proteasome inhibitor therapy, (25) percent in a blood sample of bone marrow plasma cells, (26) level in a blood sample of beta-2 microglobulin, (27) level in a blood sample of Immunoglobulin G, (28) level in a blood sample of lactate dehydrogenase, (29) ratio in a blood sample of kappa to lambda free light chain levels, (30) level in a blood sample of free light chain, (31) level in a blood sample of M-protein, (32) level in a blood sample of platelets, (33) level in a blood sample of sodium, and (34) level in a blood sample of soluble BCMA of the subject; and
(b) predicting if the subject is likely to not exhibit a clinical response to administration of the T cell therapy for treatment of the disease or condition based on one or more outputs of a process configured to predict, based on the marker or combination of markers, if the subject is likely to not exhibit the clinical response, wherein the predicting comprises providing the parameter or parameters as input to the process.
47. The method of embodiment 45 or embodiment 46, wherein the parameters of a combination of markers are obtained and provided as input to the process.
48. The method of any one of embodiments 45-47, wherein the marker is or the combination of markers comprises one or more subject immune profile markers that are selected from markers (1)-(7).
49. The method of any one of embodiments 45-48, wherein the marker is or the combination of markers comprises one or more subject fitness markers that are selected from markers (8)-(17).
50. The method of any one of embodiments 45-49, wherein the marker is or the combination of markers comprises one or more subject prior therapy markers that are selected from markers (18)-(24).
51. The method of any one of embodiments 45-50, wherein the marker is or the combination of markers comprises one or more subject tumor burden markers that are selected from markers (25)-(34).
52. A method of predicting whether a subject will exhibit a clinical response to a T cell therapy, comprising:
(a) obtaining, for a subject having a disease or condition, a parameter of a marker or parameters of a combination of markers, wherein:
   (i) the parameter or parameters are obtained prior to the subject being administered a T cell therapy comprising T cells comprising a recombinant receptor that binds to an antigen associated with the disease or condition; and
   (ii) the marker or the combination of markers is selected from any of one or more subject immune profile markers, one or more subject fitness markers, one or more subject prior therapy markers, and one or more subject tumor burden markers of the subject; and
(b) predicting if the subject is likely to exhibit a clinical response to administration of the T cell therapy for treatment of the disease or condition based on one or more outputs of a process configured to predict, based on the marker or combination of markers, if the subject is likely to exhibit the clinical response, wherein the predicting comprises providing the parameter or parameters as input to the process.
53. A method of predicting whether a subject will not exhibit a clinical response to a T cell therapy, comprising:
(a) obtaining, for a subject having a disease or condition, a parameter of a marker or parameters of a combination of markers, wherein:
   (i) the parameter or parameters are obtained prior to the subject being administered a T cell therapy comprising T cells comprising a recombinant receptor that binds to an antigen associated with the disease or condition; and
   (ii) the marker or the combination of markers is selected from any of one or more subject immune profile markers, one or more subject fitness markers, one or more subject prior therapy markers, and one or more subject tumor burden markers of the subject; and
(b) predicting if the subject is likely to not exhibit a clinical response to administration of the T cell therapy for treatment of the disease or condition based on one or more outputs of a process configured to predict, based on the marker or combination of markers, if the subject is likely to not exhibit the clinical response, wherein the predicting comprises providing the parameter or parameters as input to the process.
54. The method of embodiment 52 or embodiment 53, wherein the parameters of a combination of markers are obtained and provided as input to the process.
55. The method of any one of embodiments 52-54, wherein the combination of markers comprises one or more subject immune profile markers.
56. The method of embodiment 55, wherein the one or more subject immune profile markers are selected from the (1) level in a blood sample of d-dimer, (2) level in a blood sample of fibrinogen, (3) level in a blood sample of lymphocytes, (4) level in a blood sample of monocytes, (5) ratio in a blood sample of monocytes to leukocytes, (6) level in a blood sample of red blood cells, and (7) level in a blood sample of white blood cells of the subject.
57. The method of any one of embodiments 52-56, wherein the combination of markers comprises one or more subject fitness markers.
58. The method of embodiment 57, wherein the one or more subject fitness markers are selected from the (8) age, (9) body mass index, (10) level in a blood sample of albumin, (11) level in a blood sample of alkaline phosphatase, (12) level in a blood sample of aspartate aminotransferase, (13) level in a blood sample of alanine aminotransferase, (14) level in a blood sample of direct bilirubin, (15) level in a blood sample of bilirubin, (16) level in a blood sample of creatinine, and (17) creatinine clearance of the subject.
59. The method of any one of embodiments 52-58, wherein the combination of markers comprises one or more subject prior therapy markers.
60. The method of embodiment 59, wherein the one or more subject prior therapy markers are selected from the (18) time since diagnosis, (19) number ofpriortherapies received, (20) time since prior autologous stem cell transplant, (21) time since prior corticosteroid therapy, (22) time since prior alkylating agent therapy, (23) time since prior topoisomerase inhibitor therapy, and (24) time since prior proteasome inhibitor therapy for the subject.
61. The method of any one of embodiments 52-60, wherein the combination of markers comprises one or more subject tumor burden markers.
62. The method of embodiment 61, wherein the one or more subject tumor burden markers are selected from the (25) percent in a blood sample of bone marrow plasma cells, (26) level in a blood sample of beta-2 microglobulin, (27) level in a blood sample of Immunoglobulin G, (28) level in a blood sample of lactate dehydrogenase, (29) ratio in a blood sample of kappa to lambda free light chain levels, (30) level in a blood sample of free light chain, (31) level in a blood sample of M-protein, (32) level in a blood sample of platelets, (33) level in a blood sample of sodium, and (34) level in a blood sample of soluble BCMA of the subject.
63. The method of any one of embodiments 45-62, wherein the combination of markers comprises the (3) level in a blood sample of lymphocytes, (22) time since prior alkylating agent therapy, and (26) level in a blood sample of beta-2 microglobulin of the subject.
64. The method of any one of embodiments 45-63, wherein the combination of markers comprises the (5) ratio in a blood sample of monocytes to leukocytes, (24) time since prior proteasome inhibitor therapy, (28) level in a blood sample of lactate dehydrogenase, and (31) level in a blood sample of M-protein of the subject.
65. The method of any one of embodiments 45, 47-52, and 54-64, wherein the process comprises a machine learning model trained to predict, based on the marker or combination of markers, if the subject is likely to exhibit the clinical response.
66. The method of any one of embodiments 46-51 and 53-64, wherein the process comprises a machine learning model trained to predict, based on the marker or combination of markers, if the subject is likely to not exhibit the clinical response.
67. The method of embodiment 65 or embodiment 66, wherein the one or more outputs are outputs of, or are derived from outputs of, the machine learning model.
68. The method of any one of embodiments 65-67, wherein the machine learning model is trained using parameters of the marker or parameters of the combination of markers from a plurality of subjects that were each administered a T cell therapy comprising T cells comprising a recombinant receptor that binds to an antigen associated with a disease or condition.
69. The method of any one of embodiments 65-68, wherein the machine learning model is trained using clinical responses of the plurality of subjects following administration of the T cell therapy.
70. The method of embodiment 68 or embodiment 69, wherein the disease or condition of the plurality of subjects is the same disease or condition of the subject.
71. The method of any one of embodiments 68-70, wherein the antigen associated with the disease or condition of the plurality of subjects is the same antigen associated with the disease or condition of the subject.
72. The method of any one of embodiments 68-71, wherein the recombinant receptor of the T cell therapy of the plurality of subjects is the same recombinant receptor of the T cell therapy of the subject.
73. The method of any one of embodiments 68-72, wherein the T cell therapy of the plurality of subjects is an autologous T cell therapy.
74. The method of any one of embodiments 1-73, wherein the disease or condition is a cancer.
75. The method of any one of embodiments 1-74, wherein the disease or condition is a multiple myeloma.
76. The method of any one of embodiments 1-75, wherein the disease or condition is a relapsed/refractory multiple myeloma.
77. The method of any one of embodiments 1-76, wherein the antigen is a multiple myeloma-associated antigen.
78. The method of any one of embodiments 1-77, wherein the antigen is BCMA.
79. The method of any one of embodiments 1-78, wherein prior to the obtaining of the parameter or parameters, the subject has received one or more prior therapies for treating the disease or condition.
80. The method of embodiment 79, wherein the one or more prior therapies comprises one to three prior therapies.
81. The method of embodiment 79, wherein the one or more prior therapies comprises at least three prior therapies.
82. The method of any one of embodiments 79-81, wherein the subject has relapsed or been refractory to the most recent of the one or more prior therapies.
83. The method of any one of embodiments 79-82, wherein the one or more prior therapies comprises an immunomodulatory agent.
84. The method of embodiment 83, wherein the immunomodulatory agent is selected from thalidomide, lenalidomide, and pomalidomide.
85. The method of any one of embodiments 79-84, wherein the one or more prior therapies comprises a proteasome inhibitor.
86. The method of embodiment 85, wherein the proteasome inhibitor is selected from bortezomib, carfilzomib, and ixazomib.
87. The method of any one of embodiments 79-86, wherein the one or more prior therapies comprises an anti-CD38 antibody.
88. The method of embodiment 87, wherein the anti-CD38 antibody is or comprises daratumumab.
89. The method of any one of embodiments 1-88, wherein the clinical response is progression free survival of greater than 2 months, 4 months, 6 months, or 8 months.
90. The method of any one of embodiments 1-88, wherein the clinical response is complete response (CR).
91. The method of any one of embodiments 1-90, wherein the parameter or parameters are obtained within 6, 5, 4, 3, 2, or 1 month prior to when the T cell therapy is to be administered to the subject.
92. The method of any one of embodiments 1-91, wherein the parameter or parameters are obtained when or about when the subject is being screened for administration of the T cell therapy.
93. The method of any one of embodiments 1-92, wherein the parameter or parameters are obtained prior to when T cells for the T cell therapy are collected from the subject.
94. The method of any one of embodiments 1-93, wherein the obtaining comprises measuring the parameter or one of the more of the parameters from the subject.
95. The method of any one of embodiments 1-94, wherein the recombinant receptor is a chimeric antigen receptor (CAR).
96. The method of embodiment 95, wherein the CAR is an anti-BCMA CAR.
97. The method of embodiment 95 or embodiment 96, wherein the CAR comprises an extracellular antigen-binding domain that binds to BCMA, a transmembrane domain, and an intracellular signaling region.
98. The method of embodiment 97, wherein the intracellular signaling region comprises a cytoplasmic signaling domain of a CD3-zeta (CD3ζ) chain.
99. The method of embodiment 97 or embodiment 98, wherein the intracellular signaling region comprises a costimulatory signaling domain.
100. The method of embodiment 99, wherein the costimulatory signaling domain comprises an intracellular signaling domain of CD28, 4-1BB, or ICOS.
101. The method of embodiment 99 or embodiment 100, wherein the costimulatory signaling domain is between the transmembrane domain and the cytoplasmic signaling domain of the CD3-zeta (CD3ζ) chain.
102. The method of any of embodiments 97-101, wherein the transmembrane domain comprises a transmembrane domain from CD28 or CD8.
103. The method of any of embodiments 97-102, wherein the transmembrane domain comprises a transmembrane domain from human CD28 or CD8.
104. The method of any one of embodiments 97-103, wherein the CAR further comprises an extracellular spacer between the antigen-binding domain and the transmembrane domain.
105. The method of embodiment 104, wherein the spacer is from CD8.
106. The method of embodiment 104 or embodiment 105, wherein the spacer is a CD8alpha hinge.
107. The method of any one of embodiments 104-106, wherein the transmembrane domain and the spacer are from CD8.
108. The method of any one of embodiments 95-107, wherein the CAR comprises the sequence set forth in SEQ ID NO:38.
109. The method of any one of embodiments 1-108, wherein the T cell therapy is an autologous T cell therapy.
110. The method of any one of embodiments 1-109, wherein the T cell therapy comprises idecabtagene vicleucel cells.
111. The method of any one of embodiments 1-110, wherein the T cell therapy is ABECMA®.
112. The method of any one of embodiments 1-108, wherein the T cell therapy comprises ciltacabtagene autoleucel cells.
113. The method of any one of embodiments 1-108 and 112, wherein the T cell therapy is CARVYKTI™ 114. The method of any one of embodiments 1-113, wherein the subject is a human.
115. The method of any one of embodiments 3-9, 11-14, 16-19, 21-24, 26-29, 31-33, 36-39, 42-44, 46-51, 53-64, and 66-114, wherein the subject is predicted as likely to not exhibit the clinical response, and the method further comprises selecting the subject for administration of an alternative treatment or treatment regimen.
116. The method of any one of embodiments 1-2, 5-10, 12-15, 17-20, 22-25, 27-30, 32-35, 38-41, 44-45, 47-52, 54-65, and 67-114, wherein the subject is predicted as likely to exhibit the clinical response, and the method further comprises selecting the subject for administration of the T cell therapy.
117. The method of any one of embodiments 1-2, 5-10, 12-15, 17-20, 22-25, 27-30, 32-35, 38-41, 44-45, 47-52, 54-65, 67-114, and 116, wherein the method further comprises collecting T cells from the subject for producing the T cell therapy.
118. The method of embodiment 117, wherein the T cells are collected after the subject is predicted as likely to exhibit the clinical response.
119. The method of embodiment 117 or embodiment 118, wherein the T cells are collected by apheresis.
120. The method of any one of embodiments 117-119, wherein the T cells are collected by leukapheresis.
121. A method of treating a disease or condition in a human subject, comprising:
(a) selecting a subject having a disease or condition for administration of a T cell therapy for treating the disease or condition, the T cell therapy comprising T cells comprising a recombinant receptor that binds to an antigen associated with the disease or condition, wherein the selecting is according to the method of any one of embodiments 116-120; and
(b) administering the T cell therapy to the selected subject.
122. A method of treating a disease or condition in a human subject, comprising administering a T cell therapy to a subject having a disease or condition, wherein:
the T cell therapy comprises T cells comprising a recombinant receptor that binds to an antigen associated with the disease or condition; and
the subject is selected according to the method of any one of embodiments 116-120 for administration of the T cell therapy.
123. A method of treating a disease or condition in a human subject, comprising administering a T cell therapy to a human subject having a disease or condition, wherein:
the T cell therapy comprises T cells comprising a recombinant receptor that binds to an antigen associated with the disease or condition; and
the subject is a subject in which prior to administration of the T cell therapy to the subject, and for a marker or a combination of markers selected from (1) level in a blood sample of d-dimer, (2) level in a blood sample of fibrinogen, (3) level in a blood sample of lymphocytes, (4) level in a blood sample of monocytes, (5) ratio in a blood sample of monocytes to leukocytes, (6) level in a blood sample of red blood cells, (7) level in a blood sample of white blood cells, (8) age, (9) body mass index, (10) level in a blood sample of albumin, (11) level in a blood sample of alkaline phosphatase, (12) level in a blood sample of aspartate aminotransferase, (13) level in a blood sample of alanine aminotransferase, (14) level in a blood sample of direct bilirubin, (15) level in a blood sample of bilirubin, (16) level in a blood sample of creatinine, (17) creatinine clearance, (18) time since diagnosis, (19) number of prior therapies received, (20) time since prior autologous stem cell transplant, (21) time since prior corticosteroid therapy, (22) time since prior alkylating agent therapy, (23) time since prior topoisomerase inhibitor therapy, (24) time since prior proteasome inhibitor therapy, (25) percent in a blood sample of bone marrow plasma cells, (26) level in a blood sample of beta-2 microglobulin, (27) level in a blood sample of Immunoglobulin G, (28) level in a blood sample of lactate dehydrogenase, (29) ratio in a blood sample of kappa to lambda free light chain levels, (30) level in serum of free light chain, (31) level in a blood sample of M-protein, (32) level in a blood sample of platelets, (33) level in a blood sample of sodium, and (34) level in a blood sample of soluble BCMA of the subject:
(i) a parameter or one or more parameters of the subject for markers (3), (6)-(13), (16), (17), (20), (22)-(24), (28), (29), (32), and (33) are higher than an associated threshold level; or
(ii) a parameter or one or more parameters of the subject for markers (1), (2), (4), (5), (14), (15), (18), (19), (21), (25)-(27), (30), (31), and (34) are lower than an associated threshold level.
124. The method of embodiment 123, wherein prior to administration of the T cell therapy to the subject, the subject has been determined to have:
(i) a parameter or one or more parameters for markers (3), (6)-(13), (16), (17), (20), (22)-(24), (28), (29), (32), and (33) that are higher than an associated threshold level; or
(ii) a parameter or one or more parameters for markers (1), (2), (4), (5), (14), (15), (18), (19), (21), (25)-(27), (30), (31), and (34) that are lower than an associated threshold level.
125. A method of treating a disease or condition in a human subject, comprising administering a T cell therapy to a human subject having a disease or condition, wherein:
the T cell therapy comprises T cells comprising a recombinant receptor that binds to an antigen associated with the disease or condition; and
the subject is a subject in which prior to administration of the T cell therapy to the subject, and for a marker or a combination of markers selected from any of:
   one or more subject immune profile markers selected from (1) level in a blood sample of d-dimer, (2) level in a blood sample of fibrinogen, (3) level in a blood sample of lymphocytes, (4) level in a blood sample of monocytes, (5) ratio in a blood sample of monocytes to leukocytes, (6) level in a blood sample of red blood cells, and (7) level in a blood sample of white blood cells of the subject;
   one or more subject prior therapy markers selected from (18) time since diagnosis, (19) number of prior therapies received, (20) time since prior autologous stem cell transplant, (21) time since prior corticosteroid therapy, (22) time since prior alkylating agent therapy, (23) time since prior topoisomerase inhibitor therapy, and (24) time since prior proteasome inhibitor therapy for the subject; and
   one or more subject tumor burden markers selected from (25) percent in a blood sample of bone marrow plasma cells, (26) level in a blood sample of beta-2 microglobulin, (27) level in a blood sample of Immunoglobulin G, (28) level in a blood sample of lactate dehydrogenase, (29) ratio in a blood sample of kappa to lambda free light chain levels, (30) level in a blood sample of free light chain, (31) level in a blood sample of M-protein, (32) level in a blood sample of platelets, (33) level in a blood sample of sodium, and (34) level in a blood sample of soluble BCMA of the subject;
(i) a parameter or one or more parameters of the subject for markers (3), (6), (7), (20), (22)-(24), (28), (29), (32), and (33) are higher than an associated threshold level; or
(ii) a parameter or one or more parameters of the subject for markers (1), (2), (4), (5), (18), (19), (21), (25)-(27), (30), (31), and (34) are lower than an associated threshold level.
126. The method of embodiment 125, wherein prior to administration of the T cell therapy to the subject, the subject has been determined to have:
(i) a parameter or one or more parameters for markers (3), (6), (7), (20), (22)-(24), (28), (29), (32), and (33) that are higher than an associated threshold level; or
(ii) a parameter or one or more parameters for markers (1), (2), (4), (5), (18), (19), (21), (25)-(27), (30), (31), and (34) that are lower than an associated threshold level.
127. The method of any one of embodiments 121-126, wherein the marker is or the combination of markers comprises one or more subject immune profile markers that are selected from markers (1)-(7).
128. The method of any one of embodiments 121-124 and 127, wherein the marker is or the combination of markers comprises one or more subject fitness markers that are selected from markers (8)-(17).
129. The method of any one of embodiments 121-128, wherein the marker is or the combination of markers comprises one or more subject prior therapy markers that are selected from markers (18)-(24).
130. The method of any one of embodiments 121-129, wherein the marker is or the combination of markers comprises one or more subject tumor burden markers that are selected from markers (25)-(34).
131. The method of any one of embodiments 121-130, wherein the combination of markers comprises the (3) level in a blood sample of lymphocytes, (22) time since prior alkylating agent therapy, and (26) level in a blood sample of beta-2 microglobulin of the subject.
132. The method of embodiment 131, wherein:
(i) the parameter of the subject for marker (3) is higher than an associated threshold level;
(ii) the parameter of the subject for marker (22) is higher than an associated threshold level; or
(iii) the parameter of the subject for marker (26) is lower than an associated threshold level.
133. The method of embodiment 131 or embodiment 132, wherein:
(i) the parameter of the subject for marker (3) is higher than an associated threshold level;
(ii) the parameter of the subject for marker (22) is higher than an associated threshold level; and
(iii) the parameter of the subject for marker (26) is lower than an associated threshold level.
134. The method of any one of embodiments 121-133, wherein the combination of markers comprise the (5) ratio in a blood sample of monocytes to leukocytes, (24) time since prior proteasome inhibitor therapy, (28) level in a blood sample of lactate dehydrogenase, and (31) level in a blood sample of M-protein of the subject.
135. The method of embodiment 134, wherein:
(i) the parameter of the subject for marker (5) is lower than an associated threshold level;
(ii) the parameter of the subject for marker (24) is higher than an associated threshold level;
(iii) the parameter of the subject for marker (28) is higher than an associated threshold level; or
(iv) the parameter of the subject for marker (31) is lower than an associated threshold level.
136. The method of embodiment 134 or embodiment 135, wherein:
(i) the parameter of the subject for marker (5) is lower than an associated threshold level;
(ii) the parameter of the subject for marker (24) is higher than an associated threshold level;
(iii) the parameter of the subject for marker (28) is higher than an associated threshold level; and
(iv) the parameter of the subject for marker (31) is lower than an associated threshold level.
137. The method of any one of embodiments 121-136, wherein:
the threshold level associated with marker (1) is between or between about 0.5 mg/L and 11 mg/L or between or between about 0.5 mg/L and 1.3 mg/L;
the threshold level associated with marker (2) is between or between about 2.2 g/L and 7.7 g/L or between or between about 4.2 g/L and 5.4 g/L;
the threshold level associated with marker (3) is between or between about 0.3×10⁹ cells/L and 1.0×10⁹ cells/L or between or between about 0.4×10⁹ cells/L and 0.7×10⁹ cells/L;
the threshold level associated with marker (4) is between or between about 0.2×10⁹ cells/L and 1.1×10⁹ cells/L or between or between about 0.4×10⁹ cells/L and 0.7×10⁹ cells/L;
the threshold level associated with marker (5) is between or between about 6.7 and 18 or between or between about 13 and 14;
the threshold level associated with marker (6) is between or between about 2.4×10¹² cells/L and 3.7×10¹² cells/L or between or between about 2.9×10¹² cells/L and 3.3×10¹² cells/L;
the threshold level associated with marker (7) is between or between about 2.1×10⁹ cells/L and 7.1×10⁹ cells/L or between or between about 2.9×10⁹ cells/L and 4.2×10⁹ cells/L;
the threshold level associated with marker (8) is between or between about 57 years and 66 years or between or between about 64 years and 66 years;
the threshold level associated with marker (9) is between or between about 22 kg/m² and 31 kg/m² or between or between about 23 kg/m² and 29 kg/m²;
the threshold level associated with marker (10) is between or between about 31 g/L and 41 g/L or between or between about 36 g/L and 40 g/L;
the threshold level associated with marker (11) is between or between about 28 IU/L and 134 IU/L or between or between about 54 IU/L and 64 IU/L;
the threshold level associated with marker (12) is between or between about 7.3 IU/L and 49 IU/L or between or between about 16 IU/L and 26 IU/L;
the threshold level associated with marker (13) is between or between about 8 IU/L and 31 IU/L or between or between about 13 IU/L and 29 IU/L;
the threshold level associated with marker (14) is between or between about 1.4 μM and 2.7 μM or between or between about 1.8 μM and 2.2 μM;
the threshold level associated with marker (15) is between or between about 3.4 μM and 23 μM or between or between about 9.4 μM and 9.6 μM;
the threshold level associated with marker (16) is between or between about 46 μM and 114 μM or between or between about 52 μM and 80 μM;
the threshold level associated with marker (17) is between or between about 0.8 mL/s and 2.0 mL/s or between or between about 1.9 mL/s and 2.0 mL/s;
the threshold level associated with marker (18) is between or between about 2.2 years and 10 years or between or between about 5.5 years and 8.3 years;
the threshold level associated with marker (19) is between or between about 4 and 11 or between or between about 4 and 5;
the threshold level associated with marker (20) is between or between about 26 days and 3205 days or between or between about 641 days and 2941 days;
the threshold level associated with marker (21) is between or between about 12 days and 2257 days or between or between about 42 days and 59 days;
the threshold level associated with marker (22) is between or between about 11 days and 493 days or between or between about 230 days and 244 days;
the threshold level associated with marker (23) is between or between about 87 days and 3356 days or between or between about 474 days and 676 days;
the threshold level associated with marker (24) is between or between about 11 days and 658 days or between or between about 51 days and 170 days;
the threshold level associated with marker (25) is between or between about 21% and 100% or between or between about 56% and 80%;
the threshold level associated with marker (26) is between or between about 2.7 mg/L and 7.7 mg/L or between or between about 3.2 mg/L and 4.6 mg/L;
the threshold level associated with marker (27) is between or between about 2.8 g/L and 75 g/L or between or between about 14 g/L and 35 g/L;
the threshold level associated with marker (28) is between or between about 150 IU/L and 319 IU/L or between or between about 181 IU/L and 319 IU/L;
the threshold level associated with marker (29) is between or between about 0.003 and 763 or between or between about 8.7 and 211;
the threshold level associated with marker (30) is between or between about 0.008 g/L and 12 g/L or between or between about 0.2 g/L and 1.0 g/L;
the threshold level associated with marker (31) is between or between about 4.3 g/L and 32 g/L or between or between about 5.3 g/L and 12 g/L;
the threshold level associated with marker (32) is between or between about 53×10⁹ cells/L and 212×10⁹ cells/L or between or between about 156×10⁹ cells/L and 181×10⁹ cells/L;
the threshold level associated with marker (33) is between or between about 132 mM and 141 mM or between or between about 136 mM and 138 mM; and/or
the threshold level associated with marker (34) is between or between about 35 ng/mL and 1300 ng/mL or between or between about 170 ng/mL and 654 ng/mL.
138. The method of any one of embodiments 121-137, wherein the T cell therapy comprises between at or about 5×10⁷ recombinant receptor-comprising T cells and at or about 1×10⁹ recombinant receptor-comprising T cells or between at or about 1×10⁸ recombinant receptor-comprising T cells and at or about 1×10⁹ recombinant receptor-comprising T cells.
139. The method of any one of embodiments 121-138, wherein the T cell therapy comprises at or about 4.5×10⁹ recombinant receptor-comprising T cells.
140. The method of any one of embodiments 121-139, wherein the T cell therapy is administered by an intravenous infusion.
141. The method of any one of embodiments 121-140, wherein the T cell therapy is an autologous T cell therapy.
142. The method of any one of embodiments 121, 122, 124, and 126-141, wherein the subject is subject to apheresis to collect T cells for the T cell therapy, and wherein the selection and/or determination occurs prior to the apheresis.
143. The method of any one of embodiments 121, 122, 124, and 126-142, wherein the selection and/or determination is within 6, 5, 4, 3, 2, or 1 month prior to when the T cell therapy is administered to the subject.
144. The method of any one of embodiments 121, 122, 124, and 126-143, wherein the selection and/or determination occurs at screening of the subject for administration of the T cell therapy.
145. The method of any one of embodiments 121-144, wherein the disease or condition is a hematologic disease.
146. The method of any one of embodiments 121-145, wherein the disease or condition is a multiple myeloma.
147. The method of any one of embodiments 121-146, wherein the antigen associated with the disease or condition is human BCMA.
148. The method of any one of embodiments 121-147, wherein the T cell therapy is a CAR T cell therapy.
149. The method of any one of embodiments 121, 122, 124, and 126-148, wherein the method further comprises administering a bridging therapy to the subject, wherein the bridging therapy is administered to the subject between the selection and/or determination and the administration of the T cell therapy.

### VII. Examples

The following examples are included for illustrative purposes only and are not intended to limit the scope of the invention.

### Example 1: Construction of Exemplary BCMA CARs

A lentiviral vector construct for a chimeric antigen receptor (CAR) containing an anti-BCMA scFv antibody was designed to contain an MND promoter operably linked to an anti-BCMA scFv, a hinge and transmembrane domain from CD8alpha, and a CD137 co-stimulatory domain followed by the intracellular signaling domain of the CD3zeta chain. See, e.g., International Publication No. WO 2016/094304, which is incorporated by reference herein in its entirety, and in particular the disclosure of BCMA CARs and their characterization. The BCMA CAR contained a CD8alpha signal peptide (SP) sequence (amino acid residues 1-21) for surface expression on immune effector cells. The polynucleotide sequence of an exemplary BCMA CAR (anti-BCMA02 CAR) is set forth in SEQ ID NO: 10. The polynucleotide sequence encodes the polypeptide sequence set forth in SEQ ID NO:9, in which the mature CAR sequence starts at amino acid residue 22 of SEQ ID NO:9 (see also the mature BCMA CAR sequence set forth in SEQ ID NO:37).

Table E1 shows the identity, GenBank Reference (where applicable), Source Name and Citation for the various nucleotide segments of a BCMA CAR lentiviral vector that contained the BCMA CAR construct.

TABLE E1
Nucleotide Segments of a BCMA CAR Lentiviral Vector

| Nucleotides | Identity | GenBank Reference | Source Name | Citation |
|---|---|---|---|---|
| 1-185 | pUC19 plasmid | Accession #L09137.2 | pUC19 | New England |
| | backbone | nt 1-185 | | Biolabs |
| 185-222 | Linker | Not applicable | Synthetic | Not applicable |
| 223-800 | CMV | Not Applicable | pHCMV | Yee, et al., |
| | | | | (1994) PNAS |
| | | | | 91: 9564-68 |
| 801-1136 | R, U5, PBS, and | Accession | pNL4-3 | Maldarelli, et. al. |
| | packaging sequences | #M19921.2 | | (1991) |
| | | nt 454-789 | | J Virol: |
| | | | | 65(11): 5732-43 |
| 1137-1139 | Gag start codon | Not Applicable | Synthetic | Not applicable |
| | (ATG) changed to | | | |
| | stop codon (TAG) | | | |
| 1140-1240 | HIV-1 gag sequence | Accession | pNL4-3 | Maldarelli, et. al. |
| | | #M19921.2 | | (1991) |
| | | nt 793-893 | | J Virol: |
| | | | | 65(11): 5732-43 |
| 1241-1243 | HIV-1 gag sequence | Not Applicable | Synthetic | Not applicable |
| | changed to a second | | | |
| | stop codon | | | |
| 1244-1595 | HIV-1 gag sequence | Accession | pNL4-3 | Maldarelli, et. al. |
| | | #M19921.2 | | (1991) |
| | | nt 897-1248 | | J Virol: |
| | | | | 65(11): 5732-43 |
| 1596-1992 | HIV-1 pol | Accession | pNL4-3 | Maldarelli, et. al. |
| | cPPT/CTS | #M19921.2 | | (1991) |
| | | nt 4745-5125 | | J Virol: |
| | | | | 65(11): 5732-43 |
| 1993-2517 | HIV-1, isolate HXB3 | Accession | PgTAT-CMV | Malim, M. H. |
| | env region (RRE) | #M14100.1 | | Nature (1988) |
| | | nt 1875-2399 | | 335: 181-183 |
| 2518-2693 | HIV-1 env sequences | Accession | pNL4-3 | Maldarelli, et. al. |
| | S/A | #M19921.2 | | (1991) |
| | | nt 8290-8470 | | J Virol: |
| | | | | 65(11): 5732-43 |
| 2694-2708 | Linker | Not applicable | Synthetic | Not applicable |
| 2709-3096 | MND | Not applicable | pccl-c- | Challita et al. |
| | | | MNDU3c-x2 | (1995) |
| | | | | J. Virol. 69: |
| | | | | 748-755 |
| 3097-3124 | Linker | Not applicable | Synthetic | Not applicable |
| 3125-3187 | Signal peptide | Accession # | CD8a signal | Not applicable |
| | | NM_001768 | peptide | |
| 3188-3934 | BCMA02 scFv | Not applicable | Synthetic | Not applicable |
| 3935-4141 | CD8a hinge and TM | Accession # | CD8a hinge | Milone et al |
| | | NM_001768 | and TM | (2009) |
| | | | | Mol Ther |
| | | | | 17(8): 1453-64 |
| 4144-4269 | CD137 (4-1BB) | Accession # | CD137 | Milone et al |
| | signaling domain | NM_001561 | signaling | (2009) |
| | | | domain | Mol Ther |
| | | | | 17(8): 1453-64 |
| 4270-4606 | CD3-ζ signaling | Accession # | CD3-ζ | Milone et al |
| | domain | NM_000734 | signaling | (2009) |
| | | | domain | Mol Ther |
| | | | | 17(8): 1453-64 |
| 4607-4717 | HIV-1 ppt and part of | Accession | pNL4-3 | Maldarelli, et. al. |
| | 3′ U3 | #M19921.2 | | (1991) |
| | | nt 9005-9110 | | J Virol: |
| | | | | 65(11): 5732-43 |
| 4718-4834 | HIV-1 part of U3 | Accession | pNL4-3 | Maldarelli, et. al. |
| | (399bp deletion) and | #M19921.2 | | (1991) |
| | R | nt 9511-9627 | | J Virol: |
| | | | | 65(11): 5732-43 |
| 4835-4858 | Synthetic polyA | Not applicable | Synthetic | Levitt, N. Genes |
| | | | | & Dev (1989) |
| | | | | 3: 1019-1025 |
| 4859-4877 | Linker | Not applicable | Synthetic | Not Applicable |
| 4878-7350 | pUC19 backbone | Accession #L09137.2 | pUC19 | New England |
| | | nt 2636-2686 | | Biolabs |

### Example 2: Identification of Starting Material, Manufacturing, and Drug Product Variables Correlated with Pre-Infusion Patient Markers and Clinical Outcomes

CAR T cells expressing an anti-BCMA CAR as described in Example 1 were manufactured from peripheral blood mononuclear cells (PBMCs) isolated from leukapheresis material obtained from relapsed and refractory multiple myeloma (RRMM) patients, and then the manufactured BCMA-targeted T cells were re-administered to the patients by autologous cell therapy in a clinical trial as a third line or greater (3L+) treatment. Included patients received at least three prior regimens (including a proteasome inhibitor (PI), an immunomodulatory drug (IMiD agent), and an anti-CD38 antibody) and were refractory to their last line of therapy. Clinical and manufacturing data was harmonized across 164 RRMM patients.

Unsupervised clustering and supervised univariate and multivariate machine learning models across multiple key variable domains were trained in order to identify PBMC, drug product (DP), in-process parameter, and patient variables associated with more favorable outcomes with anti-BCMA CAR T cell therapy.

Ten selected PBMC, DP, and in-process parameters were used to define patient clusters based on manufacturing trajectories. PBMC variables included the percentage of CD3+ cells in the population of PBMCs collected by leukapheresis, the CD4 to CD8 ratio in the population of PBMCs, the percentage of CD57+ cells in the population of PBMCs, and the percentage of CD28+ cells in the population of PBMCs. CD57 can be used as a marker of senescence. CD28 can be used as a marker of less differentiation.

In-process parameter variables included the size of cells at day five (5) of manufacture and the total number of nucleated cells (TNC). Day five cell size can be used as an indicator of activation.

DP variables included the percentage of CAR-expressive cells in the resulting DP, the total number of CAR-expressive cells (CAR yield), the vector copy number (VCN; copies/μg DNA), and the CAR T cell potency.

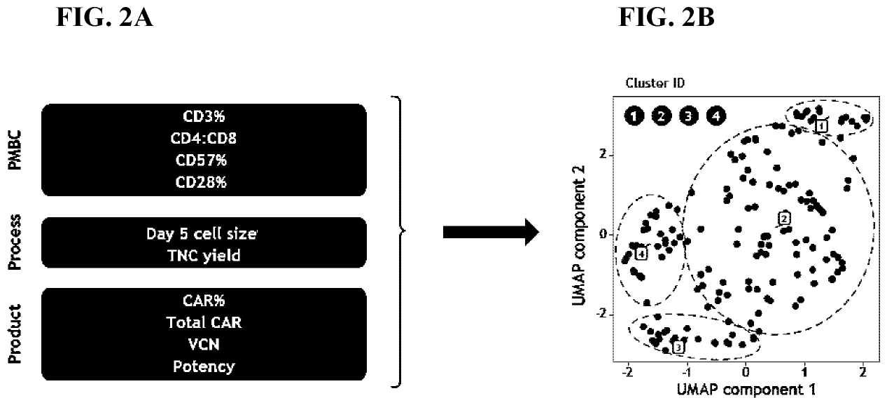

Manufacturing trajectories were defined through unsupervised clustering of Uniform Manifold Approximation and Projection (UMAP) components of the ten variables, which were dimensionally reduced using UMAP (FIG. 1). Unsupervised clustering using normal mixture modeling via expectation-maximization (EM) was applied to the first two UMAP components. Cluster number ranging from two to nine was tested, from which four clusters representing distinct manufacturing trajectories were identified as most accurately modelling the two UMAP components across patients (FIGS. 2A-2B; cluster 1, n=17; cluster 2, n=94; cluster 3, n=24; cluster 4, n=29).

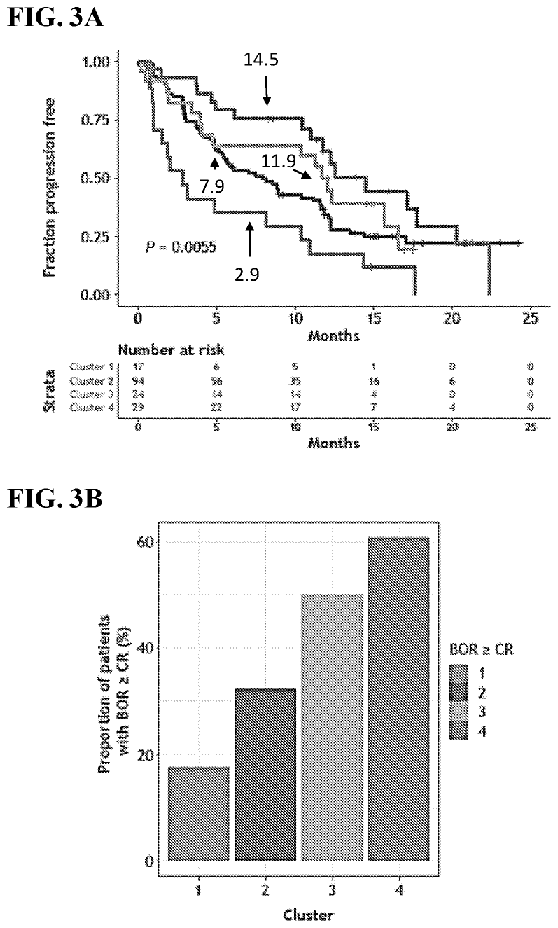

Clusters were then tested for associations with downstream clinical outcomes. A direct correlation was observed between favorable patient outcomes and manufacturing trajectories. Clusters showed significantly different clinical outcomes by median progression-free survival (mPFS), as demonstrated by Kaplan-Meier curves of PFS by manufacturing cluster (FIG. 3A). Clusters also showed significantly different clinical outcomes by complete response rate (CRR), as demonstrated by the proportion of patients with best overall response complete response or better (FIG. 3B). The patient cluster associated with the least favorable patient outcomes was cluster (1), and the patient cluster associated with the most favorable patient outcomes was cluster (4). This suggests a connection between patients with poor manufacturing profiles and less favorable clinical efficacy. In particular, patients in cluster 1 had an mPFS of 3 months and a CRR of less than 20 percent. By comparison, patients in cluster 4 had an mPFS greater than 14 months and a CRR of greater than 60 percent.

Table E2 shows the proportions of patients within each cluster who experienced cytokine release syndrome (CRS) or neurotoxicity (NT). As shown, the prevalence of safety events differed across patient clusters.

TABLE E2
Safety Events by Patient Cluster

| | 1 (n = 17) | 2 (n = 94) | 3 (n = 24) | 4 (n = 29) |
|---|---|---|---|---|
| Any CRS | 59% | 81% | 100% | 86% |
| CRS Grade ≥ 2 | 12% | 32% | 54% | 28% |
| CRS Grade ≥ 3 | 0% | 5% | 8% | 3% |
| Any NT | 12% | 14% | 42% | 14% |
| NT Grade ≥ 2 | 6% | 6% | 25% | 7% |
| NT Grade ≥ 3 | 0% | 2% | 17% | 3% |

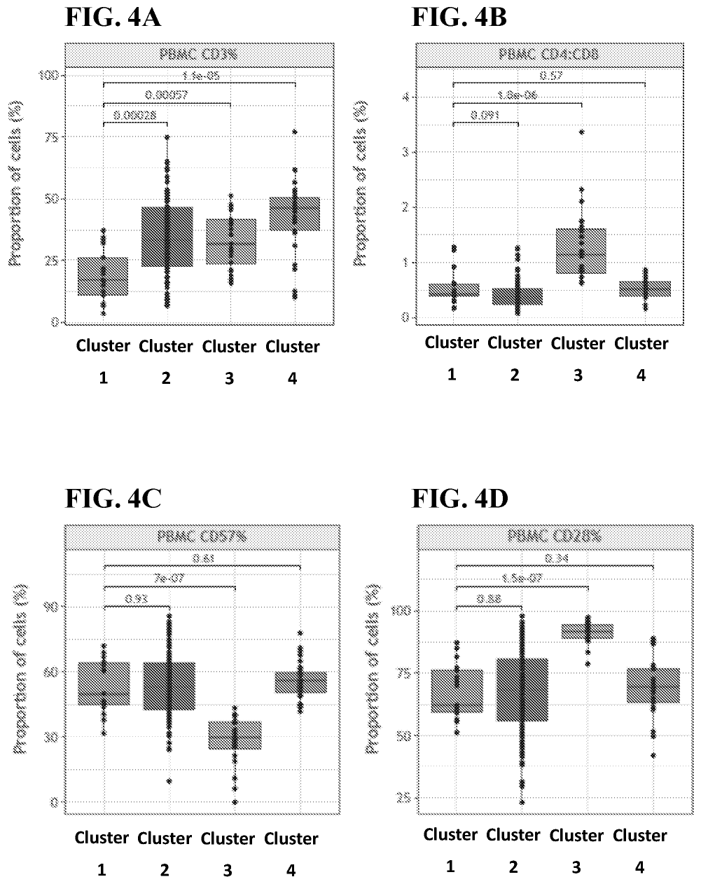

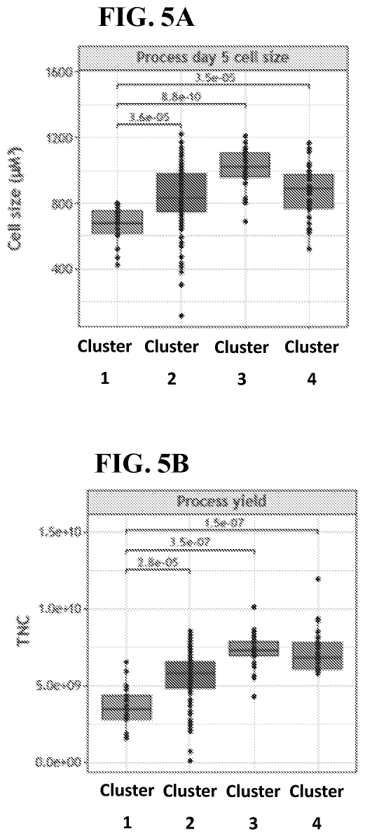

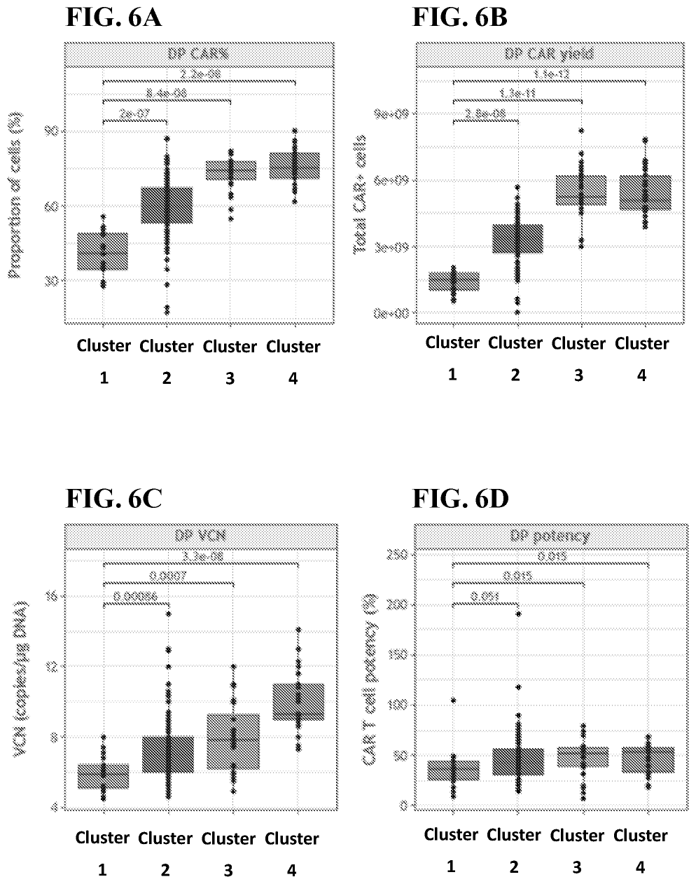

PBMC, DP, and in-process parameter values by cluster are shown in FIGS. 4A-4D, FIGS. 5A-5B, and FIGS. 6A-6D, respectively. Relative to patient cluster (1), which was associated with the least favorable patient outcomes, patient cluster (4), which was associated with the most favorable patient outcomes, was characterized by a 2-fold higher proportion of CD3+ cells in PBMCs and a better CD4 to CD8 ratio. Relative to patient cluster (1), patient cluster (4) was characterized by 25% higher day 5 cell size. Relative to patient cluster (1), patient cluster (4) had a higher proportion of cells expressing CAR, 3-fold higher CAR T yield, higher VCN, and higher potency.

Overall, out of all the patient clusters, patient cluster (4) was associated with the most favorable characteristics and exhibited a higher frequency of T cells in PBMCs, increased T cell size during manufacturing, higher DP T-cell transduction, potency, and vector copy number, and ultimately a >3-fold higher CAR T cell yield compared to the patient cluster 1, which was associated with the last favorable characteristics. Patient cluster 2 contained the majority of patients and was associated with intermediate manufacturing endpoints. Patients in cluster 4 had a higher complete response rate and longer progression free survival and had lower tumor burden, higher absolute lymphocyte count (ALC), and longer washout period after alkylator treatment among others.

Five key clinical laboratory tests measured at screening were also used in order to evaluate patient clusters based on patient profiles obtained at screening or infusion of DP. Patients' prior exposure to alkylating agents was also used. Table E3 summarizes the range of values associated with the least and most favorable patient outcomes across six patient attributes.

TABLE E3
Quantitative Patient Profiles

| | Least Favorable | | Most Favorable | |
|---|---|---|---|---|
| | Outcomes | | Outcomes | |
| | (Cluster 1) | | (Cluster 4) | |
| Albumin | 35 | g/L | 40 | g/L |
| Absolute Lymphocyte Count (ALC) | 0.5 × 10⁹/L | | 0.9 × 10⁹/L | |
| Platelets (10⁹/L) | 130 × 10⁹/L | | 190 × 10⁹/L | |
| Beta-2 Microglobulin (B2M) | 4 | mg/L | 2 | mg/L |
| Mononuclear Protein (M-Protein) | 15 | g/L | 5 | g/L |
| Prior Alkylator * | ≤6 | months | >6 | months |

* Time of last exposure is measured relative to date of apheresis.

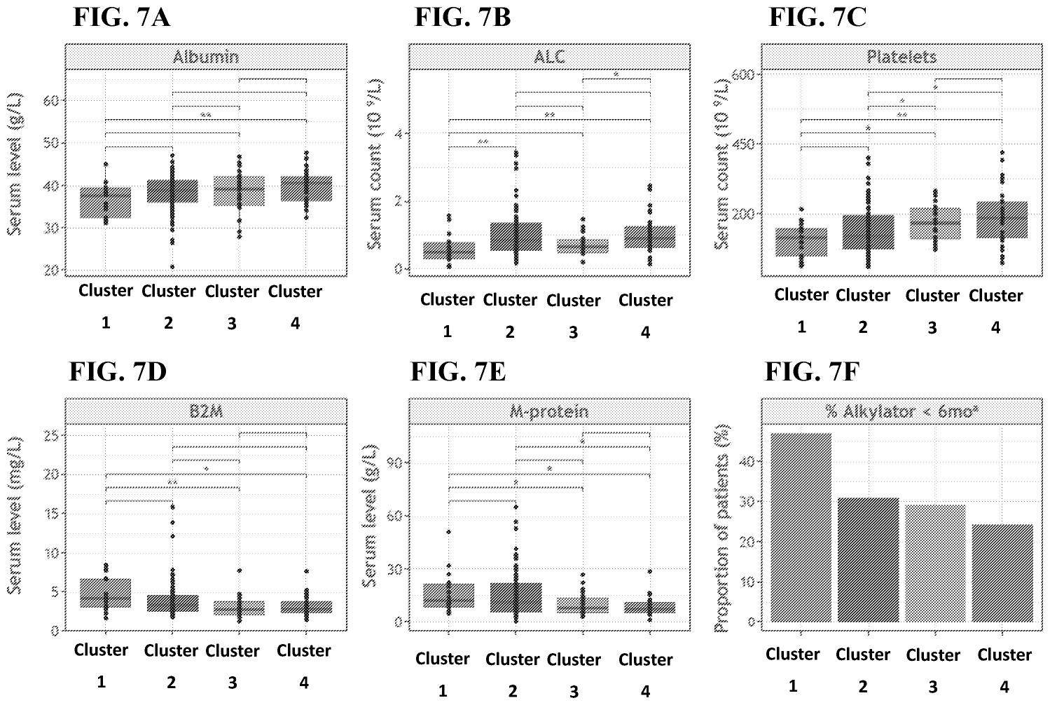

FIG. 7A shows the serum level of albumin in the different patient clusters. FIG. 7B shows the serum absolute lymphocyte count in the different patient clusters. FIG. 7C shows the serum platelet count. FIG. 7D shows the serum level of beta-2 microglobulin. FIG. 7E shows the serum level of mononuclear protein. FIG. 7F shows the proportion of patients whose last exposure to prior alkylator therapy is less than 6 months before apheresis. As shown, these attributes varied across patient clusters, including between cluster (1) and the other clusters. Of these markers, albumin can be used as a marker indicating patient fitness. ALC levels can be used as a marker indicating the immune profile (e.g., immune health) of patients. Platelet, B2M, and M-Protein levels can be used as markers indicating the tumor burden of patients. Alkylator treatment and its recency in relation to apheresis can be used as a marker related to patients' prior therapies.

Additional patient attributes that varied across patient clusters are shown in Table E4 and include serum M-protein levels, serum ALC levels, and whether or not patients had alkylator exposure within six months of apheresis. These attributes can be used as markers of the tumor burden, immune profile, and prior therapy exposure, respectively, of the patients.

TABLE E4
Patient Attributes Across Clusters

| Variable | Cluster 1 | Cluster 2 | Cluster 3 | Cluster 4 |
|---|---|---|---|---|
| Median [Q1-Q3] | N = 17 | N = 94 | N = 24 | N = 29 |
| M-protein (g/L) at | 12.08 | 10.73 | 7.36 | 7.00 |
| screening | [8.07-21.33] | [5.50-21.51] | [4.95-13.38] | [4.65-11.07] |
| ALC (10⁹/L) at screening† | 0.50 | 0.85 | 0.67 | 0.91 |
| | [0.29-0.78] | [0.54-1.36] | [0.49-0.86] | [0.62-1.26] |
| Patients with alkylator | 47 | 31 | 29 | 24 |
| exposure within 6 months | | | | |
| of apheresis (%)* | | | | |

†Assessment at screening timepoint
*Time of last exposure is measured relative to date of apheresis.

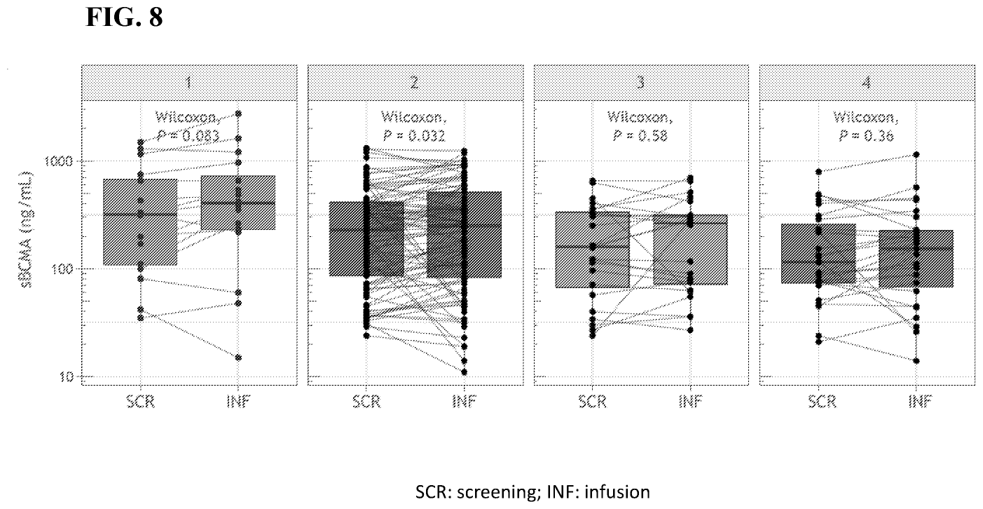

Tumor burden changes between screening and baseline, assessed by serum B-cell maturation agent (sBCMA), within each of the four patient clusters were also assessed and are shown in FIG. 8.

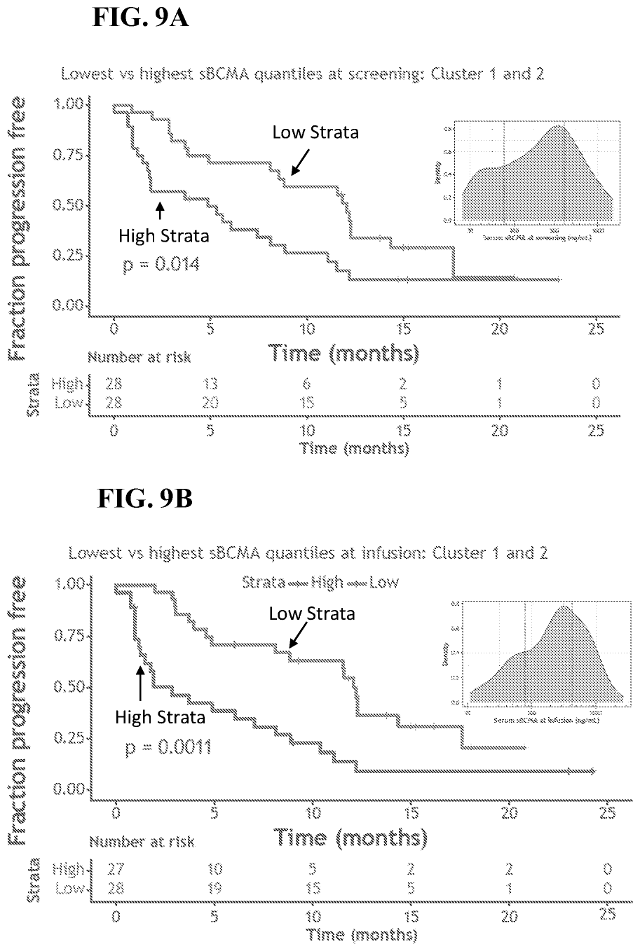

Median progression-free survival for clusters 1 and 2 can be further subdivided by highest or lowest quantile serum BCMA at screening (FIG. 9A) or at infusion (FIG. 9B). High tumor burden was a predictor of low manufacturing yield and less durable clinical response. Patients whose tumor was controlled with bridging therapy and had lower tumor burden at infusion had better outcomes than those with higher tumor burden. Even among the patient clusters associated with less favorable patient outcomes (clusters 1 and 2), patients with low tumor burden at screening had better outcomes than those with high tumor burden.

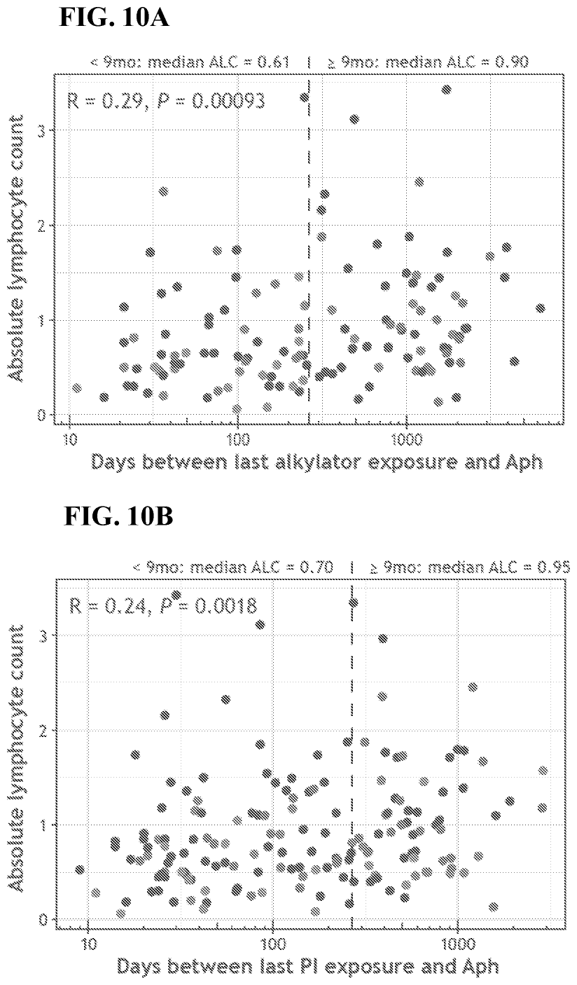

Prior exposures also impacted T cell quantity and quality in BCMA CAR T cell manufacturing outcomes. Patients with a lower total number of anti-myeloma therapies had higher quality T cells and favorable manufacturing outcomes. Neutral or favorable exposures include recent anti-CD38, immunomodulatory drugs (IMiDs), and elotuzumab. Unfavorable exposures include recent alkylator therapy (particular within 6 months), proteasome inhibitor therapy, and topoisomerase inhibitor. The correlation between absolute lymphocyte count at screening and time since last exposure to alkylating agents is shown in FIG. 10A. The correlation between absolute lymphocyte count at screening and time since last exposure to a proteasome inhibitor is shown in FIG. 10B. As shown, the length of time between prior exposure and absolute lymphocyte count were positively correlated with one another. These results suggest that consideration of factors such as low tumor burden and distancing of prior drug class exposures associated with decreased T cell quantity and quality may help to optimize outcomes for patients receiving anti-BCMA CAR T therapy.

Table E5 shows a summary of the patient, PBMC, in-process, DP, and efficacy markers in each cluster. Each marker is underlined or italicized by their modeled effect on manufacturing and clinical outcomes. Bolded, italicized markers are “least favorable” (e.g., associated with the patient outcomes of cluster 1), while CAPITALIZED, ITALICIZED markers are “most favorable” (e.g., associated with the patient outcomes of cluster 4).

TABLE E5
Qualitative Patient Profiles of Each Cluster

| Marker | Cluster 1 | Cluster 2 | Cluster 3 | Cluster 4 |
|---|---|---|---|---|
| Prior | Recent alkylator, | Recent alkylator, | Recent alkylator or | DISTANT |
| Therapies | PI, or TI Therapy | PI, or TI Therapy | PI Therapy | ALKYLATOR, PI, OR |
| | Increased | | DECREASED | TI THERAPY |
| | Number of Prior | | NUMBER OF PRIOR | |
| | Regimens | | REGIMENS | |
| Tumor | Increased soluble | Increased soluble | DECREASED | DECREASED |
| Burden | BCMA and | BCMA and | SOLUBLE BCMA | SOLUBLE BCMA |
| | mononuclear | mononuclear | AND MONONUCLEAR | AND MONONUCLEAR |
| | protein(M- | protein(M- | PROTEIN(M- | PROTEIN(M- |
| | protein) | protein) | PROTEIN) | PROTEIN) |
| | | Increased lactate | | |
| | | dehydrogenase | | |
| | | (LDH) | | |
| Immune | Decreased | INCREASED | Decreased absolute | INCREASED |
| Profile | absolute | ABSOLUTE | lymphocyte count | ABSOLUTE |
| | lymphocyte count | LYMPHOCYTE | (ALC) | LYMPHOCYTE |
| | (ALC) | COUNT(ALC) | DECREASED | COUNT(ALC) |
| | Increased | Increased | MONOCYTE:LEUKOCYTE | DECREASED |
| | monocyte:leukocyte | monocyte:leukocyte | (MONO:LEUK) | MONOCYTE:LEUKOCYTE |
| | (mono:leuk) | (mono:leuk) | RATIO | (MONO:LEUK) |
| | ratio | ratio | | RATIO |
| Patient | Decreased | Decreased | INCREASED | INCREASED |
| Fitness | albumin | creatinine | CREATININE | ALBUMIN |
| | Decreased | clearance | CLEARANCE | Decreased |
| | creatinine | | | creatinine |
| | clearance | | | clearance |
| PBMC | Decreased | Decreased ratio of | INCREASED RATIO | INCREASED |
| Material | percentage of | CD4+ to CD8+ | OF CD4+ TO CD8+ | PERCENTAGE OF |
| | CD3+ cells in | cells | CELLS | CD3+ CELLS IN |
| | PBMCs | | HIGH QUALITY | PBMCS |
| | | | PHENOTYPE(LOW | |
| | | | PERCENTAGE OF | |
| | | | CD57+ CELLS, | |
| | | | HIGH PERCENTAGE | |
| | | | OF CD28+ CELLS) | |
| In- | Decreased TNC | Decreased TNC | INCREASED TNC | INCREASED TNC |
| Process | yield | yield | YIELD | YIELD |
| | Decreased early | | INCREASED EARLY | |
| | cell size(e.g., day | | CELL SIZE | |
| | 3, 4, or 5 cell size) | | | |
| Drug | Decreased | Decreased | INCREASED | INCREASED |
| Product | percentage of | percentage of | PERCENTAGE OF | PERCENTAGE OF |
| | CD3+CAR+ cells | CD3+CAR+ cells | CD3+CAR+ CELLS | CD3+CAR+ CELLS |
| | Decreased VCN | | | INCREASED VCN |
| Efficacy | Median | Median | Median | Median |
| | progression-free | progression-free | progression-free | progression-free |
| | survival (mPFS): | survival (mPFS): | survival (mPFS): | survival (mPFS): |
| | 3 months | 7.9 months | 11.7 months | 14.5 months |
| | Complete | Complete response | Complete response | Complete response |
| | response rate | rate (CRR): 32% | rate (CRR): 50% | rate (CRR): 61% |
| | (CRR): 18% | | | |

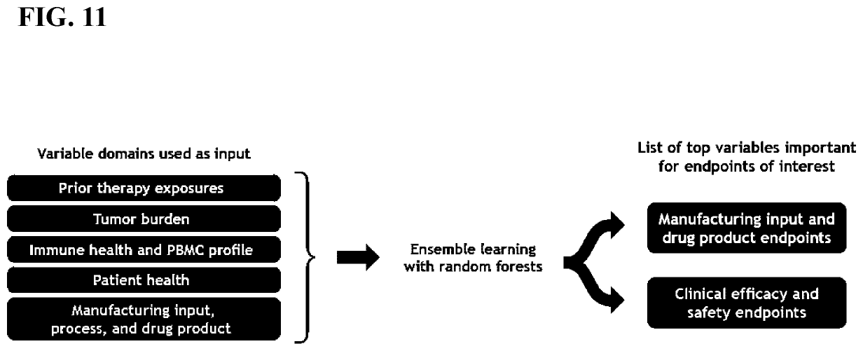

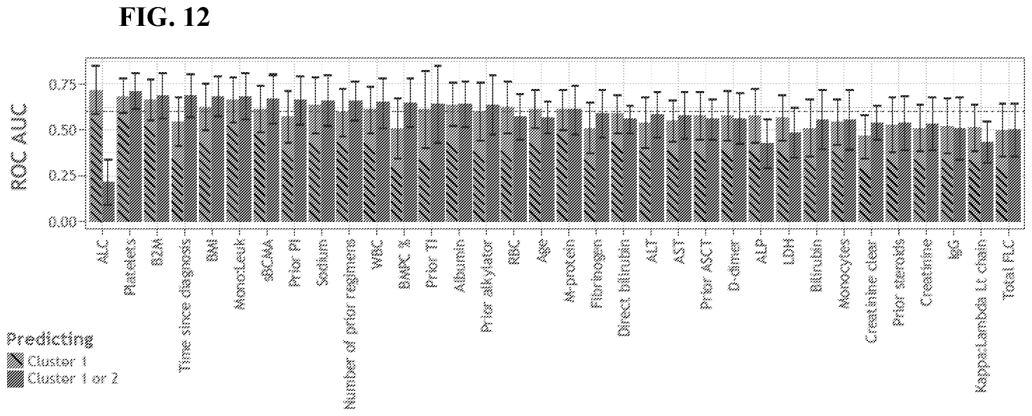

Based on the above results, supervised machine learning models (random regression and survival forests) were trained in order to predict patient outcomes and manufacturing trajectories (FIG. 11). 112 clinical, 50 PBMC, 19 manufacturing and DP variables, and 6 clinical endpoints were the markers used for model training. Multiple imputation with chained equations (MICE) was used to impute missing values. Models were created to predict whether patients were associated with clusters with less favorable patient and manufacturing outcomes. Variables with a permutation P value for importance ≤0.1 were considered for secondary analysis with Accumulated Local Effects (ALEs). Results identified key variables associated with manufacturing and clinical endpoints, their estimated effect size, and directionality of those relationships. Univariate receiver operating characteristic area under the curve values (ROC AUCs) for predicting if a patient was associated with cluster 1 or associated with clusters 1 or 2 are shown in FIG. 12.

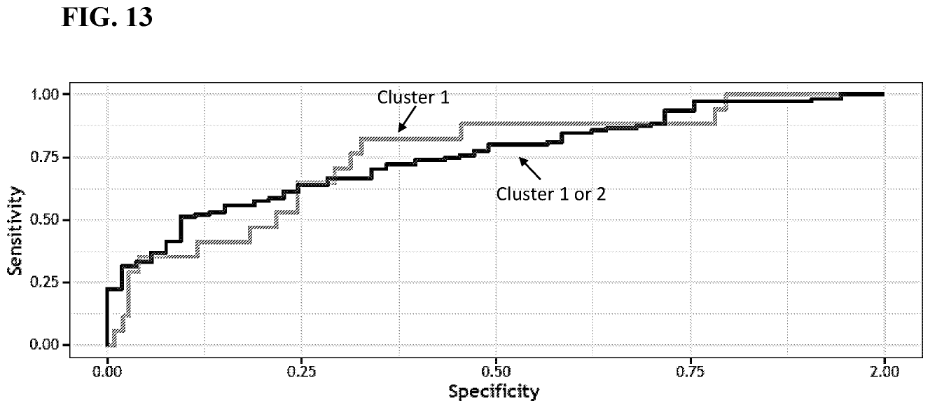

Multivariate linear models (generalized linear model, binomial family) were also developed for predicting cluster 1 or clusters 1 or 2. Optimized variable subsets for the multivariate linear models predicting cluster 1 or clusters 1 or 2 were identified using the branch-and-bound algorithm with a maximum of four variables and are shown in Table E6. Multivariate ROC curves for predicting cluster 1 or clusters 1 and 2 are shown in FIG. 13. All ROC AUCs were calculated using five-fold cross-validation.

TABLE E6
Summary Table of Optimized Variable Subsets

| | | ROC | 95% Confidence |
|---|---|---|---|
| Model | Predictors | AUC | Interval |
| Cluster 1 | ALC, B2M, and prior alkylator | 0.76 | 0.65-0.87 |
| Cluster 1 | Mono: Leuk, prior PI, | 0.75 | 0.67-0.82 |
| or 2 | M-protein, and LDH | | |

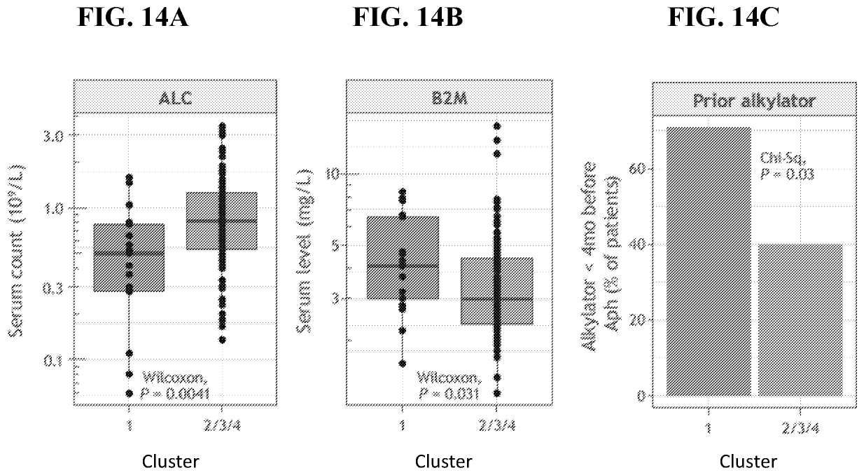

Cluster 1 predictors included serum absolute lymphocyte count (FIG. 14A), serum level of beta-2-microglobulin (FIG. 14B), and percentage of patients whose last exposure to prior alkylator therapy was less than 4 months before apheresis (FIG. 14C).

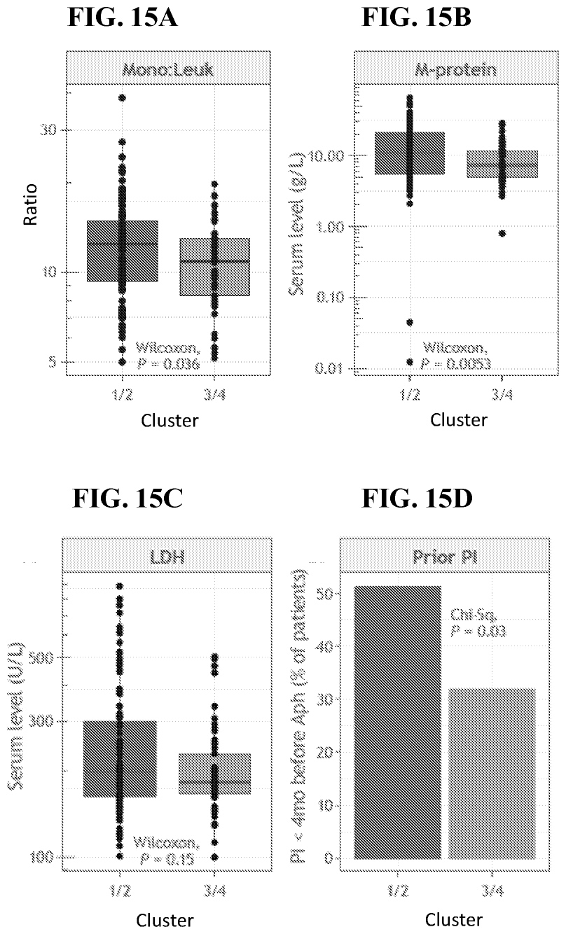

Cluster 1 or 2 predictors included the monocyte to leukocyte ratio (FIG. 15A), serum level of mononuclear protein (FIG. 15B), serum level of lactate dehydrogenase (FIG. 15C), and percentage of patients whose last exposure to prior proteasome inhibitor therapy was less than 4 months before apheresis (FIG. 15D).

In summary, patient profiles associated with “least favorable” and “most favorable” manufacturing and patient outcomes were defined for late-line RRMM patients using multivariate modeling, and correlated with clinical efficacy outcomes and safety profiles.

Together, these results show that patients who are more likely to have favorable manufacturing and efficacy outcomes may be identified by physicians using standard laboratory tests and medical history. The current study identified patient profiles in RRMM using accessible laboratory or medical history data that correlated with longitudinal outcomes. These findings may inform patients likely to achieve improved outcomes with autologous CAR T therapy. These patients share a common profile including: (1) lower tumor burden, (2) higher starting lymphocyte count, and (3) certain aspects regarding prior exposures to select therapies. Moreover, select variables including absolute lymphocyte count (ALC), beta-2 microglobulin (B2M), and time-since-last-exposure to alkylating agents may help identify patients who may be at risk for low manufacturing yield and less durable clinical efficacy.

### Example 3: Determination of Threshold Values for Predicting Clinical Outcomes from Pre-Infusion Patient Markers

Threshold values for predicting clinical outcomes from pre-infusion patient markers were determined.

Threshold values were determined using patient data from the 164 RRMM patients described in Example 2. Threshold values were determined based on their ability to discriminate between patients in cluster 1 and any other cluster (cluster 1 vs. clusters 2/3/4). Patient markers for which threshold values were determined included (1) level in serum of d-dimer, (2) level in serum of fibrinogen, (3) level in serum of lymphocytes, (4) level in serum of monocytes, (5) ratio in serum of monocytes to leukocytes, (6) level in serum of red blood cells, (7) level in serum of white blood cells, (8) age, (9) body mass index, (10) level in serum of albumin, (11) level in serum of alkaline phosphatase, (12) level in serum of aspartate aminotransferase, (13) level in serum of alanine aminotransferase, (14) level in serum of direct bilirubin, (15) level in serum of bilirubin, (16) level in serum of creatinine, (17) creatinine clearance, (18) time since diagnosis, (19) number of prior therapies received, (20) time since prior autologous stem cell transplant, (21) time since prior corticosteroid therapy, (22) time since prior alkylating agent therapy, (23) time since prior topoisomerase inhibitor therapy, (24) time since prior proteasome inhibitor therapy, (25) percent in serum of bone marrow plasma cells, (26) level in serum of beta-2 microglobulin, (27) level in serum of Immunoglobulin G, (28) level in serum of lactate dehydrogenase, (29) ratio in serum of kappa to lambda free light chain levels, (30) level in serum of free light chain, (31) level in serum of M-protein, (32) level in serum of platelets, (33) level in serum of sodium, and (34) level in serum of soluble BCMA of each patient. Threshold values were determined individually for each patient marker and each using 100 bootstrap runs of the patient data. For the data selected in each bootstrap run, the cutoff value that produced the most discrimination between cluster 1 vs. clusters 2-4 patients was determined. Cutoff values were determined based on ROC AUC values and validated for each bootstrap run using the out-of-bag samples of the bootstrap run. Table E7 provides, for each patient marker, the minimum, 25^{th} quantile, median, 75^{th} quantile, and maximum cutoff values and out-of-bag AUC ROC values across bootstrap runs. Table E7 also provides a direction indicating whether patient markers for the cluster 1 patients were less than or greater than the clusters 2-4 patients.

TABLE E7
Summary Table of Optimized Variable Subsets

| | | | | | | | ROC AUC Values | | | | |
|---|---|---|---|---|---|---|---|---|---|---|---|
| Patient | Direction | Cutoff Values | | | | | (Out-of-Bag) | | | | |
| Marker | (1 vs. 2-4) | Min | 25^{th} | Med | 75^{th} | Max | Min | 25^{th} | Med | 75^{th} | Max |
| age | < | 57 | 64 | 65 | 66 | 66 | 0.39 | 0.53 | 0.59 | 0.66 | 0.79 |
| BMI | < | 21.9 | 22.8 | 26.4 | 28.9 | 30.6 | 0.34 | 0.55 | 0.63 | 0.68 | 0.87 |
| time since | >= | 2.2 | 5.5 | 7.5 | 8.3 | 9.7 | 0.3 | 0.51 | 0.57 | 0.64 | 0.87 |
| diagnosis | | | | | | | | | | | |
| number of prior | >= | 4 | 4 | 5 | 5.25 | 11 | 0.4 | 0.54 | 0.6 | 0.65 | 0.89 |
| therapies | | | | | | | | | | | |
| bone marrow | >= | 21.4 | 56.4 | 56.4 | 80 | Inf | 0.21 | 0.43 | 0.52 | 0.6 | 0.87 |
| plasma cell | | | | | | | | | | | |
| percentage | | | | | | | | | | | |
| serum albumin | < | 31 | 35.5 | 38.6 | 39.5 | 40.8 | 0.38 | 0.59 | 0.67 | 0.72 | 0.85 |
| alkaline | < | 27.5 | 53.5 | 57.5 | 63.5 | 134 | 0.32 | 0.53 | 0.6 | 0.66 | 0.83 |
| phosphatase | | | | | | | | | | | |
| aspartate | < | 7.25 | 16 | 20.5 | 25.5 | 49 | 0.28 | 0.45 | 0.53 | 0.6 | 0.74 |
| aminotransferase | | | | | | | | | | | |
| alanine | < | 8 | 12.5 | 17.5 | 28.5 | 30.5 | 0.28 | 0.46 | 0.54 | 0.6 | 0.78 |
| aminotransferase | | | | | | | | | | | |
| beta-2 | >= | 2.7 | 3.2 | 3.7 | 4.6 | 7.7 | 0.32 | 0.58 | 0.66 | 0.72 | 0.89 |
| microglobulin | | | | | | | | | | | |
| bilirubin direct | >= | 1.37 | 1.81 | 2.22 | 2.22 | 2.74 | 0.4 | 0.56 | 0.6 | 0.65 | 0.79 |
| bilirubin | >= | 3.42 | 9.41 | 9.41 | 9.58 | 23.1 | 0.21 | 0.42 | 0.52 | 0.59 | 0.83 |
| creatinine | < | 45.5 | 52.2 | 59.7 | 79.8 | 114 | 0.2 | 0.46 | 0.53 | 0.61 | 0.81 |
| creatinine | < | 0.81 | 1.92 | 1.95 | 1.97 | 1.97 | 0.26 | 0.45 | 0.49 | 0.54 | 0.82 |
| clearance | | | | | | | | | | | |
| d-dimer | >= | 0.46 | 0.54 | 0.927 | 1.31 | 11.4 | 0.23 | 0.5 | 0.57 | 0.65 | 0.98 |
| fibrinogen | >= | 2.17 | 4.17 | 5.13 | 5.35 | 7.74 | 0.25 | 0.42 | 0.5 | 0.56 | 0.91 |
| IgG | >= | 2.76 | 13.9 | 17 | 34.8 | 75.2 | 0.25 | 0.46 | 0.53 | 0.61 | 0.86 |
| lactate | < | 150 | 181 | 246 | 319 | 319 | 0.34 | 0.51 | 0.57 | 0.63 | 0.91 |
| dehydrogenase | | | | | | | | | | | |
| absolute | < | 0.28 | 0.415 | 0.57 | 0.653 | 1.04 | 0.26 | 0.65 | 0.71 | 0.79 | 0.94 |
| lymphocyte count | | | | | | | | | | | |
| (ALC) | | | | | | | | | | | |
| kappa:lambda | < | 0.00333 | 8.74 | 17.9 | 211 | 763 | 0.23 | 0.45 | 0.51 | 0.58 | 0.76 |
| free light chain | | | | | | | | | | | |
| ratio | | | | | | | | | | | |
| free light chain | >= | 0.0082 | 0.246 | 0.713 | 0.992 | 12 | 0.19 | 0.42 | 0.49 | 0.57 | 0.81 |
| m-protein | >= | 4.27 | 5.32 | 9.67 | 12.1 | 31.7 | 0.39 | 0.57 | 0.61 | 0.66 | 0.85 |
| absolute | >= | 0.165 | 0.44 | 0.575 | 0.745 | 1.08 | 0.24 | 0.45 | 0.55 | 0.62 | 0.87 |
| monocyte count | | | | | | | | | | | |
| monocyte to | >= | 6.65 | 13.2 | 13.2 | 13.6 | 18.4 | 0.39 | 0.59 | 0.67 | 0.75 | 0.88 |
| leukocyte ratio | | | | | | | | | | | |
| platelets | < | 53 | 156 | 181 | 181 | 212 | 0.39 | 0.57 | 0.64 | 0.7 | 0.9 |
| red blood cells | < | 2.35 | 2.9 | 2.97 | 3.32 | 3.72 | 0.28 | 0.52 | 0.6 | 0.67 | 0.83 |
| white blood cells | < | 2.11 | 2.87 | 3.5 | 4.15 | 7.1 | 0.22 | 0.55 | 0.61 | 0.69 | 0.95 |
| sodium | < | 132 | 136 | 138 | 138 | 141 | 0.37 | 0.57 | 0.65 | 0.71 | 0.98 |
| soluble BCMA | >= | 35 | 170 | 315 | 654 | 1300 | 0.31 | 0.54 | 0.6 | 0.67 | 0.85 |
| time since prior | < | 26 | 641 | 1351 | 2941 | 3205 | 0.22 | 0.5 | 0.56 | 0.64 | 0.79 |
| transplant | | | | | | | | | | | |
| (ASCT) | | | | | | | | | | | |
| time since prior | >= | 12 | 42 | 55 | 59 | 2257 | 0.24 | 0.49 | 0.55 | 0.61 | 0.83 |
| corticosteroid | | | | | | | | | | | |
| time since prior | < | 11 | 230 | 244 | 244 | 493 | 0.11 | 0.54 | 0.61 | 0.66 | 0.9 |
| alkylating agent | | | | | | | | | | | |
| time since | < | 87 | 474 | 676 | 676 | 3356 | 0.31 | 0.51 | 0.58 | 0.68 | 0.88 |
| topoisomerase | | | | | | | | | | | |
| inhibitor | | | | | | | | | | | |
| time since | < | 11 | 51 | 87 | 170 | 658 | 0.26 | 0.51 | 0.59 | 0.67 | 0.9 |
| proteasome | | | | | | | | | | | |
| inhibitor | | | | | | | | | | | |

These results validate that threshold values for patient markers can be used to identify patients that had poorer manufacturing and clinical outcomes (e.g., patient cluster 1), compared to others (e.g., patient clusters 2-4).

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

Sequences

| SEQ ID | | |
|---|---|---|
| NO. | Sequence | Description |
| 1 | RASESVTILGSHLIH | CDR-L1 |
| 2 | LASNVQT | CDR-L2 |
| 3 | LQSRTIPRT | CDR-L3 |
| 4 | DYSIN | CDR-H1 |
| 5 | WINTETREPAYAYDFRG | CDR-H2 |
| 6 | DYSYAMDY | CDR-H3 |
| 7 | DIVLTQSPPSLAMSLGKRATISCRASESVTILGSHLIHWYQQKPGQP | Variable light |
| | PTLLIQLASNVQTGVPARFSGSGSRTDFTLTIDPVEEDDVAVYYCL | (VL) Anti- |
| | QSRTIPRTFGGGTKLEIK | BCMA |
| 8 | QIQLVQSGPELKKPGETVKISCKASGYTFTDYSINWVKRAPGKGLK | Variable |
| | WMGWINTETREPAYAYDFRGRFAFSLETSASTAYLQINNLKYEDT | heavy (VH) |
| | ATYFCALDYSYAMDYWGQGTSVTVSS | Anti-BCMA |
| 9 | MALPVTALLLPLALLLHAARPDIVLTQSPPSLAMSLGKRATISCRA | BCMA CAR |
| | SESVTILGSHLIHWYQQKPGQPPTLLIQLASNVQTGVPARFSGSGSR | (with signal |
| | TDFTLTIDPVEEDDVAVYYCLQSRTIPRTFGGGTKLEIKGSTSGSGK | sequence) |
| | PGSGEGSTKGQIQLVQSGPELKKPGETVKISCKASGYTFTDYSINW | (aa) |
| | VKRAPGKGLKWMGWINTETREPAYAYDFRGRFAFSLETSASTAY | |
| | LQINNLKYEDTATYFCALDYSYAMDYWGQGTSVTVSSAAATTTP | |
| | APRPPTPAPTIASQPLSLRPEACRPAAGGAVHTRGLDFACDIYIWAP | |
| | LAGTCGVLLLSLVITLYCKRGRKKLLYIFKQPFMRPVQTTQEEDGC | |
| | SCRFPEEEEGGCELRVKFSRSADAPAYQQGQNQLYNELNLGRREE | |
| | YDVLDKRRGRDPEMGGKPRRKNPQEGLYNELQKDKMAEAYSEIG | |
| | MKGERRRGKGHDGLYQGLSTATKDTYDALHMQALPPR | |
| 10 | atggcactccccgtcaccgcccttctcttgcccctcgccctgctgctgcatgctgccaggcccgacattgt | BCMA CAR |
| | gctcactcagtcacctcccagcctggccatgagcctgggaaaaagggccaccatctcctgtagagccag | (with signal |
| | tgagtccgtcacaatcttggggagccatcttattcactggtatcagcagaagcccgggcagcctccaacc | sequence) |
| | cttcttattcagctcgcgtcaaacgtccagacgggtgtacctgccagattttctggtagcgggtcccgcact | (nt) |
| | gattttacactgaccatagatccagtggaagaagacgatgtggccgtgtattattgtctgcagagcagaac | |
| | gattcctcgcacatttggtgggggtactaagctggagattaagggaagcacgtccggctcagggaagcc | |
| | gggctccggcgagggaagcacgaaggggcaaattcagctggtccagagcggacctgagctgaaaaa | |
| | acccggcgagactgttaagatcagttgtaaagcatctggctataccttcaccgactacagcataaattgggt | |
| | gaaacgggcccctggaaagggcctcaaatggatgggttggatcaataccgaaactagggagcctgctta | |
| | tgcatatgacttccgcgggagattcgccttttcactcgagacatctgcctctactgcttacctccaaataaac | |
| | aacctcaagtatgaagatacagccacttacttttgcgccctcgactatagttacgccatggactactgggga | |
| | cagggaacctccgttaccgtcagttccgcggccgcaaccacaacacctgctccaaggccccccacacc | |
| | cgctccaactatagccagccaaccattgagcctcagacctgaagcttgcaggcccgcagcaggaggcg | |
| | ccgtccatacgcgaggcctggacttcgcgtgtgatatttatatttgggcccctttggccggaacatgtgggg | |
| | tgttgcttctctcccttgtgatcactctgtattgtaagcgcgggagaaagaagctcctgtacatcttcaagca | |
| | gccttttatgcgacctgtgcaaaccactcaggaagaagatgggtgttcatgccgcttccccgaggaggaa | |
| | gaaggagggtgtgaactgagggtgaaattttctagaagcgccgatgctcccgcatatcagcagggtcag | |
| | aatcagctctacaatgaattgaatctcggcaggcgagaagagtacgatgttctggacaagagacggggc | |
| | agggatcccgagatggggggaaagccccggagaaaaaatcctcaggaggggttgtacaatgagctgc | |
| | agaaggacaagatggctgaagcctatagcgagatcggaatgaaaggcgaaagacgcagaggcaagg | |
| | ggcatgacggtctgtaccagggtctctctacagccaccaaggacacttatgatgcgttgcatatgcaagcc | |
| | ttgccaccccgctaatga | |
| 11 | MLQMAGQCSQNEYFDSLLHACIPCQLRCSSNTPPLTCQRYCNASV | Human |
| | TNSVKGTNAILWTCLGLSLIISLAVFVLMFLLRKINSEPLKDEFKNT | BCMA |
| | GSGLLGMANIDLEKSRTGDEIILPRGLEYTVEECTCEDCIKSKPKVD | |
| | SDHCFPLPAMEEGATILVTTKTNDYCKSLPAALSATEIEKSISAR | |
| 12 | DGGGS | Linker |
| 13 | TGEKP | Linker |
| 14 | GGRR | Linker |
| 15 | GGGGS | Linker |
| 16 | EGKSSGSGSESKVD | Linker |
| 17 | KESGSVSSEQLAQFRSLD | Linker |
| 18 | GGRRGGGS | Linker |
| 19 | LRQRDGERP | Linker |
| 20 | LRQKDGGGSERP | Linker |
| 21 | LRQKDGGGSGGGSERP | Linker |
| 22 | GSTSGSGKPGSGEGSTKG | Linker |
| 23 | EX₁X₂YX₃QX₄ | Protease |
| | X₁ is Any amino acid | cleavage site |
| | X₂ is Any amino acid | |
| | X₃ is Any amino acid | |
| | X₄ is Gly or Ser | |
| 24 | ENLYFQG | Protease |
| | | cleavage site |
| 25 | ENLYFQS | Protease |
| | | cleavage site |
| 26 | LLNFDLLKLAGDVESNPGP | Self-cleaving |
| | | polypeptide |
| | | site |
| 27 | TLNFDLLKLAGDVESNPGP | Self-cleaving |
| | | polypeptide |
| 28 | LLKLAGDVESNPGP | Self-cleaving |
| | | polypeptide |
| 29 | NFDLLKLAGDVESNPGP | Self-cleaving |
| | | polypeptide |
| 30 | QLLNFDLLKLAGDVESNPGP | Self-cleaving |
| | | polypeptide |
| 31 | APVKQTLNFDLLKLAGDVESNPGP | Self-cleaving |
| | | polypeptide |
| 32 | VTELLYRMKRAETYCPRPLLAIHPTEARHKQKIVAPVKQT | Self-cleaving |
| | | polypeptide |
| 33 | LNFDLLKLAGDVESNPGP | Self-cleaving |
| | | polypeptide |
| 34 | LLAIHPTEARHKQKIVAPVKQTLNFDLLKLAGDVESNPGP | Self-cleaving |
| | | polypeptide |
| 35 | EARHKQKIVAPVKQTLNFDLLKLAGDVESNPGP | Self-cleaving |
| | | polypeptide |
| 36 | tcgcgcgtttcggtgatgacggtgaaaacctctgacacatgcagctcccggagacggtcacagcttgtct | Vector |
| | gtaagcggatgccgggagcagacaagcccgtcagggcgcgtcagcgggtgttggcgggtgtcgggg | encodinganti- |
| | ctggcttaactatgcggcatcagagcagattgtactgagagtgcaccatcatatgccagcctatggtgaca | BCMA CAR |
| | ttgattattgactagttattaatagtaatcaattacggggtcattagttcatagcccatatatggagttccgcgtt | |
| | acataacttacggtaaatggcccgcctggctgaccgcccaacgacccccgcccattgacgtcaataatga | |
| | cgtatgttcccatagtaacgccaatagggactttccattgacgtcaatgggtggagtatttacggtaaactgc | |
| | ccacttggcagtacatcaagtgtatcatatgccaagtacgccccctattgacgtcaatgacggtaaatggcc | |
| | cgcctggcattatgcccagtacatgaccttatgggactttcctacttggcagtacatctacgtattagtcatcg | |
| | ctattaccatggtgatgcggttttggcagtacatcaatgggcgtggatagcggtttgactcacggggatttc | |
| | caagtctccaccccattgacgtcaatgggagtttgttttggcaccaaaatcaacgggactttccaaaatgtc | |
| | gtaacaactccgccccattgacgcaaatgggcggtaggcgtgtacggtgggaggtctatataagcagag | |
| | ctcgtttagtgaaccgggtctctctggttagaccagatctgagcctgggagctctctggctaactagggaa | |
| | cccactgcttaagcctcaataaagcttgccttgagtgctcaaagtagtgtgtgcccgtctgttgtgtgactct | |
| | ggtaactagagatccctcagacccttttagtcagtgtggaaaatctctagcagtggcgcccgaacaggga | |
| | cttgaaagcgaaagtaaagccagaggagatctctcgacgcaggactcggcttgctgaagcgcgcacgg | |
| | caagaggcgaggggcggcgactggtgagtacgccaaaaattttgactagcggaggctagaaggagag | |
| | agtagggtgcgagagcgtcggtattaagcgggggagaattagataaatgggaaaaaattcggttaaggc | |
| | cagggggaaagaaacaatataaactaaaacatatagttagggcaagcagggagctagaacgattcgca | |
| | gttaatcctggccttttagagacatcagaaggctgtagacaaatactgggacagctacaaccatcccttcag | |
| | acaggatcagaagaacttagatcattatataatacaatagcagtcctctattgtgtgcatcaaaggatagatg | |
| | taaaagacaccaaggaagccttagataagatagaggaagagcaaaacaaaagtaagaaaaaggcaca | |
| | gcaagcagcagctgacacaggaaacaacagccaggtcagccaaaattaccctatagtgcagaacctcc | |
| | aggggcaaatggtacatcaggccatatcacctagaactttaaattaagacagcagtacaaatggcagtatt | |
| | catccacaattttaaaagaaaaggggggattggggggtacagtgcaggggaaagaatagtagacataat | |
| | agcaacagacatacaaactaaagaattacaaaaacaaattacaaaaattcaaaattttcgggtttattacag | |
| | ggacagcagagatccagtttggaaaggaccagcaaagctcctctggaaaggtgaaggggcagtagtaa | |
| | tacaagataatagtgacataaaagtagtgccaagaagaaaagcaaagatcatcagggattatggaaaaca | |
| | gatggcaggtgatgattgtgtggcaagtagacaggatgaggattaacacatggaaaagattagtaaaaca | |
| | ccatagctctagagcgatcccgatcttcagacctggaggaggagatatgagggacaattggagaagtga | |
| | attatataaatataaagtagtaaaaattgaaccattaggagtagcacccaccaaggcaaagagaagagtg | |
| | gtgcagagagaaaaaagagcagtgggaataggagctttgttccttgggttcttgggagcagcaggaagc | |
| | actatgggcgcagcgtcaatgacgctgacggtacaggccagacaattattgtctggtatagtgcagcagc | |
| | agaacaatttgctgagggctattgaggcgcaacagcatctgttgcaactcacagtctggggcatcaagca | |
| | gctccaggcaagaatcctggctgtggaaagatacctaaaggatcaacagctcctggggatttggggttgc | |
| | tctggaaaactcatttgcaccactgctgtgccttggaatgctagttggagtaataaatctctggaacagattt | |
| | ggaatcacacgacctggatggagtgggacagagaaattaacaattacacaagcttggtaggtttaagaat | |
| | agtttttgctgtactttctatagtgaatagagttaggcagggatattcaccattatcgtttcagacccacctccc | |
| | aaccccgaggggacccgacaggcccgaaggaatagaagaagaaggtggagagagagacagagaca | |
| | gatccattcgattagtgaacggatccatctcgacggaatgaaagaccccacctgtaggtttggcaagctag | |
| | gatcaaggttaggaacagagagacagcagaatatgggccaaacaggatatctgtggtaagcagttcctg | |
| | ccccggctcagggccaagaacagttggaacagcagaatatgggccaaacaggatatctgtggtaagca | |
| | gttcctgccccggctcagggccaagaacagatggtccccagatgcggtcccgccctcagcagtttctag | |
| | agaaccatcagatgtttccagggtgccccaaggacctgaaatgaccctgtgccttatttgaactaaccaatc | |
| | agttcgcttctcgcttctgttcgcgcgcttctgctccccgagctcaataaaagagcccacaacccctcactc | |
| | ggcgcgattcacctgacgcgtctacgccaccatggcactccccgtcaccgcccttctcttgcccctcgccc | |
| | tgctgctgcatgctgccaggcccgacattgtgctcactcagtcacctcccagcctggccatgagcctggg | |
| | aaaaagggccaccatctcctgtagagccagtgagtccgtcacaatcttggggagccatcttattcactggt | |
| | atcagcagaagcccgggcagcctccaacccttcttattcagctcgcgtcaaacgtccagacgggtgtacc | |
| | tgccagattttctggtagcgggtcccgcactgattttacactgaccatagatccagtggaagaagacgatgt | |
| | ggccgtgtattattgtctgcagagcagaacgattcctcgcacatttggtgggggtactaagctggagattaa | |
| | gggaagcacgtccggctcagggaagccgggctccggcgagggaagcacgaaggggcaaattcagct | |
| | ggtccagagcggacctgagctgaaaaaacccggcgagactgttaagatcagttgtaaagcatctggctat | |
| | accttcaccgactacagcataaattgggtgaaacgggcccctggaaagggcctcaaatggatgggttgg | |
| | atcaataccgaaactagggagcctgcttatgcatatgacttccgcgggagattcgccttttcactcgagaca | |
| | tctgcctctactgcttacctccaaataaacaacctcaagtatgaagatacagccacttacttttgcgccctcg | |
| | actatagttacgccatggactactggggacagggaacctccgttaccgtcagttccgcggccgcaaccac | |
| | aacacctgctccaaggccccccacacccgctccaactatagccagccaaccattgagcctcagacctga | |
| | agcttgcaggcccgcagcaggaggcgccgtccatacgcgaggcctggacttcgcgtgtgatatttatattt | |
| | gggcccctttggccggaacatgtggggtgttgcttctctcccttgtgatcactctgtattgtaagcgcggga | |
| | gaaagaagctcctgtacatcttcaagcagccttttatgcgacctgtgcaaaccactcaggaagaagatgg | |
| | gtgttcatgccgcttccccgaggaggaagaaggagggtgtgaactgagggtgaaattttctagaagcgc | |
| | cgatgctcccgcatatcagcagggtcagaatcagctctacaatgaattgaatctcggcaggcgagaaga | |
| | gtacgatgttctggacaagagacggggcagggatcccgagatggggggaaagccccggagaaaaaat | |
| | cctcaggaggggttgtacaatgagctgcagaaggacaagatggctgaagcctatagcgagatcggaat | |
| | gaaaggcgaaagacgcagaggcaaggggcatgacggtctgtaccagggtctctctacagccaccaag | |
| | gacacttatgatgcgttgcatatgcaagccttgccaccccgctaatgacaggtacctttaagaccaatgact | |
| | tacaaggcagctgtagatcttagccactttttaaaagaaaaggggggactggaagggctaattcactccca | |
| | aagaagacaagatctgctttttgcctgtactgggtctctctggttagaccagatctgagcctgggagctctct | |
| | ggctaactagggaacccactgcttaagcctcaataaagcttgccttgagtgcttcaatgtgtgtgttggttttt | |
| | tgtgtgtcgaaattctagcgattctagcttggcgtaatcatggtcatagctgtttcctgtgtgaaattgttatcc | |
| | gctcacaattccacacaacatacgagccggaagcataaagtgtaaagcctggggtgcctaatgagtgag | |
| | ctaactcacattaattgcgttgcgctcactgcccgctttccagtcgggaaacctgtcgtgccagctgcattaa | |
| | tgaatcggccaacgcgcggggagaggcggtttgcgtattgggcgctcttccgcttcctcgctcactgact | |
| | cgctgcgctcggtcgttcggctgcggcgagcggtatcagctcactcaaaggcggtaatacggttatccac | |
| | agaatcaggggataacgcaggaaagaacatgtgagcaaaaggccagcaaaaggccaggaaccgtaa | |
| | aaaggccgcgttgctggcgtttttccataggctccgcccccctgacgagcatcacaaaaatcgacgctca | |
| | agtcagaggtggcgaaacccgacaggactataaagataccaggcgtttccccctggaagctccctcgtg | |
| | cgctctcctgttccgaccctgccgcttaccggatacctgtccgcctttctcccttcgggaagcgtggcgcttt | |
| | ctcatagctcacgctgtaggtatctcagttcggtgtaggtcgttcgctccaagctgggctgtgtgcacgaac | |
| | cccccgttcagcccgaccgctgcgccttatccggtaactatcgtcttgagtccaacccggtaagacacga | |
| | cttatcgccactggcagcagccactggtaacaggattagcagagcgaggtatgtaggcggtgctacaga | |
| | gttcttgaagtggtggcctaactacggctacactagaagaacagtatttggtatctgcgctctgctgaagcc | |
| | agttaccttcggaaaaagagttggtagctcttgatccggcaaacaaaccaccgctggtagcggtggtttttt | |
| | tgtttgcaagcagcagattacgcgcagaaaaaaaggatctcaagaagatcctttgatcttttctacggggtc | |
| | tgacgctcagtggaacgaaaactcacgttaagggattttggtcatgagattatcaaaaaggatcttcaccta | |
| | gatccttttaaattaaaaatgaagttttaaatcaatctaaagtatatatgagtaaacttggtctgacagttacca | |
| | atgcttaatcagtgaggcacctatctcagcgatctgtctatttcgttcatccatagttgcctgactccccgtcgt | |
| | gtagataactacgatacgggagggcttaccatctggccccagtgctgcaatgataccgcgagacccacg | |
| | ctcaccggctccagatttatcagcaataaaccagccagccggaagggccgagcgcagaagtggtcctg | |
| | caactttatccgcctccatccagtctattaattgttgccgggaagctagagtaagtagttcgccagttaatagt | |
| | ttgcgcaacgttgttgccattgctacaggcatcgtggtgtcacgctcgtcgtttggtatggcttcattcagctc | |
| | cggttcccaacgatcaaggcgagttacatgatcccccatgttgtgcaaaaaagcggttagctccttcggtc | |
| | ctccgatcgttgtcagaagtaagttggccgcagtgttatcactcatggttatggcagcactgcataattctctt | |
| | actgtcatgccatccgtaagatgcttttctgtgactggtgagtactcaaccaagtcattctgagaatagtgtat | |
| | gcggcgaccgagttgctcttgcccggcgtcaatacgggataataccgcgccacatagcagaactttaaaa | |
| | gtgctcatcattggaaaacgttcttcggggcgaaaactctcaaggatcttaccgctgttgagatccagttcg | |
| | atgtaacccactcgtgcacccaactgatcttcagcatcttttactttcaccagcgtttctgggtgagcaaaaa | |
| | caggaaggcaaaatgccgcaaaaaagggaataagggcgacacggaaatgttgaatactcatactcttccttt | |
| | ttcaatattattgaagcatttatcagggttattgtctcatgagcggatacatatttgaatgtatttagaaaaat | |
| | aaacaaataggggttccgcgcacatttccccgaaaagtgccacctgggactagctttttgcaaaagcctag | |
| | gcctccaaaaaagcctcctcactacttctggaatagctcagaggccgaggcggcctcggcctctgcataa | |
| | ataaaaaaaattagtcagccatggggcggagaatgggcggaactgggcggagttaggggcgggatgg | |
| | gcggagttaggggcgggactatggttgctgactaattgagatgagcttgcatgccgacattgattattgact | |
| | agtccctaagaaaccattcttatcatgacattaacctataaaaataggcgtatcacgaggccctttcgtc | |
| 37 | DIVLTQSPPSLAMSLGKRATISCRASESVTILGSHLIHWYQQKPGQP | anti-BCMA |
| | PTLLIQLASNVQTGVPARFSGSGSRTDFTLTIDPVEEDDVAVYYCL | CAR |
| | QSRTIPRTFGGGTKLEIKGSTSGSGKPGSGEGSTKGQIQLVQSGPEL | (without |
| | KKPGETVKISCKASGYTFTDYSINWVKRAPGKGLKWMGWINTET | signal |
| | REPAYAYDFRGRFAFSLETSASTAYLQINNLKYEDTATYFCALDYS | sequence) |
| | YAMDYWGQGTSVTVSSAAATTTPAPRPPTPAPTIASQPLSLRPEAC | |
| | RPAAGGAVHTRGLDFACDIYIWAPLAGTCGVLLLSLVITLYCKRG | |
| | RKKLLYIFKQPFMRPVQTTQEEDGCSCRFPEEEEGGCELRVKFSRS | |
| | ADAPAYQQGQNQLYNELNLGRREEYDVLDKRRGRDPEMGGKPR | |
| | RKNPQEGLYNELQKDKMAEAYSEIGMKGERRRGKGHDGLYQGL | |
| | STATKDTYDALHMQALPPR | |
| 38 | DIVLTQSPPSLAMSLGKRATISCRASESVTILGSHLIHWYQQKPGQP | anti-BCMA |
| | PTLLIQLASNVQTGVPARFSGSGSRTDFTLTIDPVEEDDVAVYYCL | scFv |
| | QSRTIPRTFGGGTKLEIKGSTSGSGKPGSGEGSTKGQIQLVQSGPEL | |
| | KKPGETVKISCKASGYTFTDYSINWVKRAPGKGLKWMGWINTET | |
| | REPAYAYDFRGRFAFSLETSASTAYLQINNLKYEDTATYFCALDYS | |
| | YAMDYWGQGTSVTVSS | |
| 39 | ESKYGPPCPPCP | spacer |
| | | (IgG4hinge) |
| | | (aa) |
| | | Homo |
| | | sapiens |
| 40 | GAATCTAAGTACGGACCGCCCTGCCCCCCTTGCCCT | spacer |
| | | (IgG4hinge) |
| | | (nt) |
| | | Homo |
| | | sapiens |
| 41 | ESKYGPPCPPCPGQPREPQVYTLPPSQEEMTKNQVSLTCLVKGFYP | Hinge-CH3 |
| | SDIAVEWESNGQPENNYKTTPPVLDSDGSFFLYSRLTVDKSRWQE | spacer |
| | GNVFSCSVMHEALHNHYTQKSLSLSLGK | Homo |
| | | sapiens |
| 42 | ESKYGPPCPPCPAPEFLGGPSVFLFPPKPKDTLMISRTPEVTCVVVD | Hinge-CH2- |
| | VSQEDPEVQFNWYVDGVEVHNAKTKPREEQFNSTYRVVSVLTVL | CH3 spacer |
| | HQDWLNGKEYKCKVSNKGLPSSIEKTISKAKGQPREPQVYTLPPS | Homo |
| | QEEMTKNQVSLTCLVKGFYPSDIAVEWESNGQPENNYKTTPPVLD | sapiens |
| | SDGSFFLYSRLTVDKSRWQEGNVFSCSVMHEALHNHYTQKSLSLS | |
| | LGK | |
| 43 | RWPESPKAQASSVPTAQPQAEGSLAKATTAPATTRNTGRGGEEKK | IgD-hinge-Fc |
| | KEKEKEEQEERETKTPECPSHTQPLGVYLLTPAVQDLWLRDKATF | Homo |
| | TCFVVGSDLKDAHLTWEVAGKVPTGGVEEGLLERHSNGSQSQHS | sapiens |
| | RLTLPRSLWNAGTSVTCTLNHPSLPPQRLMALREPAAQAPVKLSL | |
| | NLLASSDPPEAASWLLCEVSGFSPPNILLMWLEDQREVNTSGFAPA | |
| | RPPPQPGSTTFWAWSVLRVPAPPSPQPATYTCVVSHEDSRTLLNAS | |
| | RSLEVSYVTDH | |
| 44 | LEGGGEGRGSLLTCGDVEENPGPR | T2A |
| | | artificial |
| 45 | RKVCNGIGIGEFKDSLSINATNIKHFKNCTSISGDLHILPVAFRGDSF | tEGFR |
| | THTPPLDPQELDILKTVKEITGFLLIQAWPENRTDLHAFENLEIIRGR | artificial |
| | TKQHGQFSLAVVSLNITSLGLRSLKEISDGDVIISGNKNLCYANTIN | |
| | WKKLFGTSGQKTKIISNRGENSCKATGQVCHALCSPEGCWGPEPR | |
| | DCVSCRNVSRGRECVDKCNLLEGEPREFVENSECIQCHPECLPQA | |
| | MNITCTGRGPDNCIQCAHYIDGPHCVKTCPAGVMGENNTLVWKY | |
| | ADAGHVCHLCHPNCTYGCTGPGLEGCPTNGPKIPSIATGMVGALL | |
| | LLLVVALGIGLFM | |
| 46 | FWVLVVVGGVLACYSLLVTVAFIIFWV | CD28 (amino |
| | | acids 153- |
| | | 179 of |
| | | Accession |
| | | No. P10747) |
| | | Homo |
| | | sapiens |
| 47 | IEVMYPPPYLDNEKSNGTIIHVKGKHLCPSPLFPGPSKPFWVLVVV | CD28 (amino |
| | GGVLACYSLLVTVAFIIFWV | acids 114- |
| | | 179 of |
| | | Accession |
| | | No. P10747) |
| | | Homo |
| | | sapiens |
| 48 | RSKRSRLLHSDYMNMTPRRPGPTRKHYQPYAPPRDFAAYRS | CD28 (amino |
| | | acids 180- |
| | | 220 of |
| | | P10747) |
| | | Homo |
| | | sapiens |
| 49 | RSKRSRGGHSDYMNMTPRRPGPTRKHYQPYAPPRDFAAYRS | CD28 (LL to |
| | | GG) |
| | | Homo |
| | | sapiens |
| 50 | KRGRKKLLYIFKQPFMRPVQTTQEEDGCSCRFPEEEEGGCEL | 4-1BB |
| | | (amino acids |
| | | 214-255 of |
| | | Q07011.1) |
| | | Homo |
| | | sapiens |
| 51 | RVKFSRSADAPAYQQGQNQLYNELNLGRREEYDVLDKRRGRDPE | CD3 zeta |
| | MGGKPRRKNPQEGLYNELQKDKMAEAYSEIGMKGERRRGKGHD | Homo |
| | GLYQGLSTATKDTYDALHMQALPPR | sapiens |
| 52 | RVKFSRSAEPPAYQQGQNQLYNELNLGRREEYDVLDKRRGRDPE | CD3 zeta |
| | MGGKPRRKNPQEGLYNELQKDKMAEAYSEIGMKGERRRGKGHD | Homo |
| | GLYQGLSTATKDTYDALHMQALPPR | sapiens |
| 53 | RVKFSRSADAPAYKQGQNQLYNELNLGRREEYDVLDKRRGRDPE | CD3 zeta |
| | MGGKPRRKNPQEGLYNELQKDKMAEAYSEIGMKGERRRGKGHD | Homo |
| | GLYQGLSTATKDTYDALHMQALPPR | sapiens |
| 54 | PGGG-(SGGGG)5-P- wherein P is proline, G is glycine and S is serine | Linker |
| 55 | GSADDAKKDAAKKDGKS | Linker |
| 56 | QIQLVQSGPDLKKPGETVKLSCKASGYTFTNFGMNWVKQAPGKG | Variable |
| | FKWMAWINTYTGESYFADDFKGRFAFSVETSATTAYLQINNLKTE | heavy (VH) |
| | DTATYFCARGEIYYGYDGGFAYWGQGTLVTVSA | Anti-BCMA |
| 57 | DVVMTQSHRFMSTSVGDRVSITCRASQDVNTAVSWYQQKPGQSP | Variable light |
| | KLLIFSASYRYTGVPDRFTGSGSGADFTLTISSVQAEDLAVYYCQQ | (VL) Anti- |
| | HYSTPWTFGGGTKLDIK | BCMA |
| 58 | EVQLVQSGAEVKKPGESLKISCKGSGYSFTSYWIGWVRQMPGKGL | Variable |
| | EWMGIIYPGDSDTRYSPSFQGHVTISADKSISTAYLQWSSLKASDT | heavy (VH) |
| | AMYYCARYSGSFDNWGQGTLVTVSS | Anti-BCMA |
| 59 | SYELTQPPSASGTPGQRVTMSCSGTSSNIGSHSVNWYQQLPGTAPK | Variable light |
| | LLIYTNNQRPSGVPDRFSGSKSGTSASLAISGLQSEDEADYYCAAW | (VL) Anti- |
| | DGSLNGLVFGGGTKLTVLG | BCMA |
| 60 | EVQLVQSGAEMKKPGASLKLSCKASGYTFIDYYVYWMRQAPGQ | Variable |
| | GLESMGWINPNSGGTNYAQKFQGRVTMTRDTSISTAYMELSRLRS | heavy (VH) |
| | DDTAMYYCARSQRDGYMDYWGQGTLVTVSS | Anti-BCMA |
| 61 | QSALTQPASVSASPGQSIAISCTGTSSDVGWYQQHPGKAPKLMIYE | Variable light |
| | DSKRPSGVSNRFSGSKSGNTASLTISGLQAEDEADYYCSSNTRSST | (VL) Anti- |
| | LVFGGGTKLTVLG | BCMA |
| 62 | GGGS | Linker |
| 63 | GGGGSGGGGSGGGGS | Linker |
| 64 | SRGGGGSGGGGSGGGGSLEMA | Linker |
| 65 | ESKYGPPCPPCPAPPVAGPSVFLFPPKPKDTLMISRTPEVTCVVVDV | Hinge-CH2- |
| | SQEDPEVQFNWYVDGVEVHNAKTKPREEQFQSTYRVVSVLTVLH | CH3 spacer |
| | QDWLNGKEYKCKVSNKGLPSSIEKTISKAKGQPREPQVYTLPPSQE | Homo |
| | EMTKNQVSLTCLVKGFYPSDIAVEWESNGQPENNYKTTPPVLDSD | sapiens |
| | GSFFLYSRLTVDKSRWQEGNVFSCSVMHEALHNHYTQKSLSLSLG | |
| | K | |
| 66 | EVQLVQSGAEVKKPGSSVKVSCKASGGTFSSYAISWVRQAPGQGL | Variable |
| | EWMGRIIPILGIANYAQKFQGRVTMTEDTSTDTAYMELSSLRSEDT | heavy (VH) |
| | AVYYCARSGYSKSIVSYMDYWGQGTLVTVSS | Anti-BCMA |
| 67 | LPVLTQPPSTSGTPGQRVTVSCSGSSSNIGSNVVFWYQQLPGTAPK | Variable light |
| | LVIYRNNQRPSGVPDRFSVSKSGTSASLAISGLRSEDEADYYCAAW | (VL) Anti- |
| | DDSLSGYVFGTGTKVTVLG | BCMA |
| 68 | QVQLVQSGAEVKKPGSSVKVSCKASGGTFSSYAISWVRQAPGQGL | Variable |
| | EWMGRIIPILGTANYAQKFQGRVTITADESTSTAYMELSSLRSEDT | heavy (VH) |
| | AVYYCARSGYGSYRWEDSWGQGTLVTVSS | Anti-BCMA |
| 69 | QAVLTQPPSASGTPGQRVTISCSGSSSNIGSNYVFWYQQLPGTAPK | Variable light |
| | LLIYSNNQRPSGVPDRFSGSKSGTSASLAISGLRSEDEADYYCAAW | (VL) Anti- |
| | DDSLSASYVFGTGTKVTVLG | BCMA |
| 70 | QVQLVQSGAEVKKPGASVKVSCKASGYTFTDYYMHWVRQAPGQ | Variable |
| | RLEWMGWINPNSGGTNYAQKFQDRITVTRDTSSNTGYMELTRLR | heavy (VH) |
| | SDDTAVYYCARSPYSGVLDKWGQGTLVTVSS | Anti-BCMA |
| 71 | QSVLTQPPSVSGAPGQRVTISCTGSSSNIGAGFDVHWYQQLPGTAP | Variable light |
| | KLLIYGNSNRPSGVPDRFSGSKSGTSASLAITGLQAEDEADYYCQS | (VL) Anti- |
| | YDSSLSGYVFGTGTKVTVLG | BCMA |
| 72 | ASGGGGSGGRASGGGGS | Linker |
| 73 | MALPVTALLLPLALLLHAARP | CD8a signal |
| | | peptide |
| 74 | METDTLLLWVLLLWVPGSTG | signal |
| | | peptide |
| 75 | EVQLLESGGGLVQPGGSLRLSCAASGFTFSSYAMSWVRQAPGKGL | Variable |
| | EWVSAISGSGGSTYYADSVKGRFTISRDNSKNTLYLQMNSLRAED | heavy (VH) |
| | TAVYYCARAEMGAVFDIWGQGTMVTVSS | Anti-BCMA |
| 76 | EIVLTQSPATLSLSPGERATLSCRASQSVSRYLAWYQQKPGQAPRL | Variable light |
| | LIYDASNRATGIPARFSGSGSGTDFTLTISSLEPEDFAVYYCQQRIS | (VL) Anti- |
| | WPFTFGGGTKVEIK | BCMA |
| 77 | QVQLVESGGGVVQPGRSLRLSCAASGFTFSSYGMHWVRQAPGKG | Variable |
| | LEWVAVISYDGSNKYYADSVKGRFTISRDNSKNTLYLQMNSLRAE | heavy (VH) |
| | DTAVYYCARDGTYLGGLWYFDLWGRGTLVTVSS | Anti-BCMA |
| 78 | DIVMTQSPLSLPVTPGEPASISCRSSQSLLHSNGYNYLDWYLQKPG | Variable light |
| | QSPQLLIYLGSNRASGVPDRFSGSGSGTDFTLKISRVEAEDVGVYY | (VL) Anti- |
| | CMQGLGLPLTFGGGTKVEIK | BCMA |
| 79 | QVQLVQSGAEVKKPGASVKVSCKASGYTFTSYYMHWVRQAPGQ | Variable |
| | GLEWMGIINPGGGSTSYAQKFQGRVTMTRDTSTSTVYMELSSLRS | heavy (VH) |
| | EDTAVYYCARESWPMDVWGQGTTVTVSS | Anti-BCMA |
| 80 | EIVMTQSPATLSVSPGERATLSCRASQSVSSNLAWYQQKPGQAPR | Variable light |
| | LLIYGASTRATGIPARFSGSGSGTEFTLTISSLQSEDFAVYYCQQYA | (VL) Anti- |
| | AYPTFGGGTKVEIK | BCMA |
| 81 | QLQLQESGPGLVKPSETLSLTCTVSGGSISSSSYYWGWIRQPPGKG | Variable |
| | LEWIGSISYSGSTYYNPSLKSRVTISVDTSKNQFSLKLSSVTAADTA | heavy (VH) |
| | VYYCARGRGYATSLAFDIWGQGTMVTVSS | Anti-BCMA |
| 82 | EIVLTQSPATLSLSPGERATLSCRASQSVSSYLAWYQQKPGQAPRL | Variable light |
| | LIYDASNRATGIPARFSGSGSGTDFTLTISSLEPEDFAVYYCQQRHV | (VL) Anti- |
| | WPPTFGGGTKVEIK | BCMA |
| 83 | EVQLVESGGGLVQPGGSLRLSCAASGFTFSSYSMNWVRQAPGKG | Variable |
| | LEWVSTISSSSSTIYYADSVKGRFTISRDNAKNSLYLQMNSLRAED | heavy (VH) |
| | TAVYYCARGSQEHLIFDYWGQGTLVTVSS | Anti-BCMA |
| 84 | EIVLTQSPATLSLSPGERATLSCRASQSVSRYLAWYQQKPGQAPRL | Variable light |
| | LIYDASNRATGIPARFSGSGSGTDFTLTISSLEPEDFAVYYCQQRFY | (VL) Anti- |
| | YPWTFGGGTKVEIK | BCMA |
| 85 | QVQLVESGGGVVQPGRSLRLSCAASGFTFSSYGMHWVRQAPGKG | Variable |
| | LEWVAVISYDGSNKYYADSVKGRFTISRDNSKNTLYLQMNSLRAE | heavy (VH) |
| | DTAVYYCARTDFWSGSPPGLDYWGQGTLVTVSS | Anti-BCMA |
| 86 | DIQLTQSPSSVSASVGDRVTITCRASQGISSWLAWYQQKPGKAPKL | Variable light |
| | LIYGASSLQSGVPSRFSGSGSGTDFTLTISSLQPEDFATYYCQQIYTF | (VL) Anti- |
| | PFTFGGGTKVEIK | BCMA |
| 87 | QVQLVQSGAEVKKPGSSVKVSCKASGGTFSSYAISWVRQAPGQGL | Variable |
| | EWMGGIIPIFGTANYAQKFQGRVTITADESTSTAYMELSSLRSEDT | heavy (VH) |
| | AVYYCARTPEYSSSIWHYYYGMDVWGQGTTVTVSS | Anti-BCMA |
| 88 | DIVMTQSPDSLAVSLGERATINCKSSQSVLYSSNNKNYLAWYQQK | Variable light |
| | PGQPPKLLIYWASTRESGVPDRFSGSGSGTDFTLTISSLQAEDVAV | (VL) Anti- |
| | YYCQQFAHTPFTFGGGTKVEIK | BCMA |
| 89 | QVQLVESGGGVVQPGRSLRLSCAASGFTFSSYGMHWVRQAPGKG | Variable |
| | LEWVAVISYDGSNKYYADSVKGRFTISRDNSKNTLYLQMNSLRAE | heavy (VH) |
| | DTAVYYCVKGPLQEPPYDYGMDVWGQGTTVTVSS | Anti-BCMA |
| 90 | EIVMTQSPATLSVSPGERATLSCRASQSVSSNLAWYQQKPGQAPR | Variable light |
| | LLIYSASTRATGIPARFSGSGSGTEFTLTISSLQSEDFAVYYCQQHH | (VL) Anti- |
| | VWPLTFGGGTKVEIK | BCMA |
| 91 | QVQLVQSGAEVKKPGSSVKVSCKASGGTFSSYAISWVRQAPGQGL | Variable |
| | EWMGRIIPILGIANYAQKFQGRVTITADKSTSTAYMELSSLRSEDT | heavy (VH) |
| | AVYYCARGGYYSHDMWSEDWGQGTLVTVSS | Anti-BCMA |
| 92 | LPVLTQPPSASGTPGQRVTISCSGRSSNIGSNSVNWYRQLPGAAPK | Variable light |
| | LLIYSNNQRPPGVPVRFSGSKSGTSASLAISGLQSEDEATYYCATW | (VL) Anti- |
| | DDNLNVHYVFGTGTKVTVLG | BCMA |
| 93 | QVQLVQSGSELKKPGASVKVSCKASGYTFTDYSINWVRQAPGQG | Variable |
| | LEWMGWINTETREPAYAYDFRGRFVFSLDTSVSTAYLQISSLKAE | heavy (VH) |
| | DTAVYYCARDYSYAMDYWGQGTLVTVSS | Anti-BCMA |
| 94 | DIVLTQSPASLAVSLGERATINCRASESVSVIGAHLIHWYQQKPGQ | Variable light |
| | PPKLLIYLASNLETGVPARFSGSGSGTDFTLTISSLQAEDAAIYYCL | (VL) Anti- |
| | QSRIFPRTFGQGTKLEIK | BCMA |
| 95 | EVQLVESGGGLVQPGGSLRLSCAVSGFALSNHGMSWVRRAPGKG | Variable |
| | LEWVSGIVYSGSTYYAASVKGRFTISRDNSRNTLYLQMNSLRPED | heavy (VH) |
| | TAIYYCSAHGGESDVWGQGTTVTVSS | Anti-BCMA |
| 96 | DIQLTQSPSSLSASVGDRVTITCRASQSISSYLNWYQQKPGKAPKLL | Variable light |
| | IYAASSLQSGVPSRFSGSGSGTDFTLTISSLQPEDFATYYCQQSYSTP | (VL) Anti- |
| | YTFGQGTKVEIK | BCMA |
| 97 | QVQLVESGGGLVQPGRSLRLSCAASGFTFSNYAMSWVRQAPGKG | Variable |
| | LGWVSGISRSGENTYYADSVKGRFTISRDNSKNTLYLQMNSLRDE | heavy (VH) |
| | DTAVYYCARSPAHYYGGMDVWGQGTTVTVSS | Anti-BCMA |
| 98 | DIVLTQSPGTLSLSPGERATLSCRASQSISSSFLAWYQQKPGQAPRL | Variable light |
| | LIYGASRRATGIPDRFSGSGSGTDFTLTISRLEPEDSAVYYCQQYHS | (VL) Anti- |
| | SPSWTFGQGTKLEIK | BCMA |
| 99 | QVQLVESGGGLVQPGGSLRLSCAVSGFALSNHGMSWVRRAPGKG | Variable |
| | LEWVSGIVYSGSTYYAASVKGRFTISRDNSRNTLYLQMNSLRPED | heavy (VH) |
| | TAIYYCSAHGGESDVWGQGTTVTVSS | Anti-BCMA |
| 100 | DIRLTQSPSPLSASVGDRVTITCQASEDINKFLNWYHQTPGKAPKL | Variable light |
| | LIYDASTLQTGVPSRFSGSGSGTDFTLTINSLQPEDIGTYYCQQYES | (VL) Anti- |
| | LPLTFGGGTKVEIK | BCMA |
| 101 | EVQLVESGGGLVQPGGSLRLSCAVSGFALSNHGMSWVRRAPGKG | Variable |
| | LEWVSGIVYSGSTYYAASVKGRFTISRDNSRNTLYLQMNSLRPED | heavy (VH) |
| | TAIYYCSAHGGESDVWGQGTTVTVSS | Anti-BCMA |
| 102 | EIVLTQSPGTLSLSPGERATLSCRASQSIGSSSLAWYQQKPGQAPRL | Variable light |
| | LMYGASSRASGIPDRFSGSGSGTDFTLTISRLEPEDFAVYYCQQYA | (VL) Anti- |
| | GSPPFTFGQGTKVEIK | BCMA |
| 103 | QIQLVQSGPELKKPGETVKISCKASGYTFRHYSMNWVKQAPGKGL | Variable |
| | KWMGRINTESGVPIYADDFKGRFAFSVETSASTAYLVINNLKDED | heavy (VH) |
| | TASYFCSNDYLYSLDFWGQGTALTVSS | Anti-BCMA |
| 104 | DIVLTQSPPSLAMSLGKRATISCRASESVTILGSHLIYWYQQKPGQP | Variable light |
| | PTLLIQLASNVQTGVPARFSGSGSRTDFTLTIDPVEEDDVAVYYCL | (VL) Anti- |
| | QSRTIPRTFGGGTKLEIK | BCMA |
| 105 | QIQLVQSGPELKKPGETVKISCKASGYTFTHYSMNWVKQAPGKGL | Variable |
| | KWMGRINTETGEPLYADDFKGRFAFSLETSASTAYLVINNLKNED | heavy (VH) |
| | TATFFCSNDYLYSCDYWGQGTTLTVSS | Anti-BCMA |
| 106 | DIVLTQSPASLAMSLGKRATISCRASESVSVIGAHLIHWYQQKPGQ | Variable light |
| | PPKLLIYLASNLETGVPARFSGSGSGTDFTLTIDPVEEDDVAIYSCL | (VL) Anti- |
| | QSRIFPRTFGGGTKLEIK | BCMA |
| 107 | QVQLVQSGAEVKKPGASVKVSCKASGYSFPDYYINWVRQAPGQG | Variable |
| | LEWMGWIYFASGNSEYNQKFTGRVTMTRDTSINTAYMELSSLTSE | heavy (VH) |
| | DTAVYFCASLYDYDWYFDVWGQGTMVTVSS | Anti-BCMA |
| 108 | DIVMTQTPLSLSVTPGQPASISCKSSQSLVHSNGNTYLHWYLQKPG | Variable light |
| | QSPQLLIYKVSNRFSGVPDRFSGSGSGTDFTLKISRVEAEDVGIYYC | (VL) Anti- |
| | SQSSIYPWTFGQGTKLEIK | BCMA |
| 109 | QVQLVQSGAEVKKPGASVKVSCKASGYSFPDYYINWVRQAPGQG | Variable |
| | LEWMGWIYFASGNSEYNQKFTGRVTMTRDTSSSTAYMELSSLRSE | heavy (VH) |
| | DTAVYFCASLYDYDWYFDVWGQGTMVTVSS | Anti-BCMA |
| 110 | DIVMTQTPLSLSVTPGEPASISCKSSQSLVHSNGNTYLHWYLQKPG | Variable light |
| | QSPQLLIYKVSNRFSGVPDRFSGSGSGADFTLKISRVEAEDVGVYY | (VL) Anti- |
| | CAETSHVPWTFGQGTKLEIK | BCMA |
| 111 | QVQLVESGGGLVQPGGSLRLSCEASGFTLDYYAIGWFRQAPGKER | Anti-BCMA |
| | EGVICISRSDGSTYYADSVKGRFTISRDNAKKTVYLQMISLKPEDT | sdAb |
| | AAYYCAAGADCSGYLRDYEFRGQGTQVTVSS | |
| 112 | IEVMYPPPYLDNEKSNGTIIHVKGKHLCPSPLFPGPSKP | CD28 spacer |
| 113 | IYIWAPLAGTCGVLLLSLVITLYCN | CD8a TM |
| 114 | LDNEKSNGTIIHVKGKHLCPSPLFPGPSKP | CD28 spacer |
| | | (truncated) |
| 115 | PTTTPAPRPPTPAPTIASQPLSLRPEACRPAAGGAVHTRGLDFACD | CD8a hinge |
| 116 | TTTPAPRPPTPAPTIASQPLSLRPEACRPAAGGAVHTRGLDFACD | CD8a hinge |
| 117 | FVPVFLPAKPTTTPAPRPPTPAPTIASQPLSLRPEACRPAAGGAVHT | CD8a hinge |
| | RGLDFACD | |
| 118 | DTGLYICKVELMYPPPYYLGIGNGTQIYVIDPEPCPDSD | CTLA4 |
| | | hinge |
| 119 | FLLWILAAVSSGLFFYSFLLTAVS | CTLA4 TM |
| 120 | QIKESLRAELRVTERRAEVPTAHPSPSPRPAGQFQTLV | PD-1 hinge |
| 121 | VGVVGGLLGSLVLLVWVLAVI | PD-1 TM |
| 122 | GLAVSTISSFFPPGYQ | Fc(gamma)R |
| | | IIIa hinge |
| 123 | EPKSPDKTHTCPPCPAPPVAGPSVFLFPPKPKDTLMIARTPEVTCVV | IgG1 hinge |
| | VDVSHEDPEVKFNWYVDGVEVHNAKTKPREEQYNSTYRVVSVLT | |
| | VLHQDWLNGKEYKCKVSNKALPAPIEKTISKAKGQPREPQVYTLP | |
| | PSRDELTKNQVSLTCLVKGFYPSDIAVEWESNGQPENNYKTTPPVL | |
| | DSDGSFFLYSKLTVDKSRWQQGNVFSCSVMHEALHNHYTQKSLS | |
| | LSPGK | |
| 124 | EVQLLESGGGLVQPGGSLRLSCAASGFTFSSYAMSWVRQAPGKGL | anti-BCMA |
| | EWVSAISGSGGSTYYADSVKGRFTISRDNSKNTLYLQMNSLRAED | CAR |
| | TAVYYCARAEMGAVFDIWGQGTMVTVSSGSTSGSGKPGSGEGST | |
| | KGEIVLTQSPATLSLSPGERATLSCRASQSVSRYLAWYQQKPGQAP | |
| | RLLIYDASNRATGIPARFSGSGSGTDFTLTISSLEPEDFAVYYCQQRI | |
| | SWPFTFGGGTKVEIKRAAALDNEKSNGTIIHVKGKHLCPSPLFPGP | |
| | SKPFWVLVVVGGVLACYSLLVTVAFIIFWVRSKRSRLLHSDYMN | |
| | MTPRRPGPTRKHYQPYAPPRDFAAYRSRVKFSRSADAPAYQQGQ | |
| | NQLYNELNLGRREEYDVLDKRRGRDPEMGGKPRRKNPQEGLYNE | |
| | LQKDKMAEAYSEIGMKGERRRGKGHDGLYQGLSTATKDTYDAL | |
| | HMQALPPR | |
| 125 | EIVLTQSPATLSLSPGERATLSCRASQSVSRYLAWYQQKPGQAPRL | anti-BCMA |
| | LIYDASNRATGIPARFSGSGSGTDFTLTISSLEPEDFAVYYCQQRIS | CAR |
| | WPFTFGGGTKVEIKRGSTSGSGKPGSGEGSTKGEVQLLESGGGLV | |
| | QPGGSLRLSCAASGFTFSSYAMSWVRQAPGKGLEWVSAISGSGGS | |
| | TYYADSVKGRFTISRDNSKNTLYLQMNSLRAEDTAVYYCARAEM | |
| | GAVFDIWGQGTMVTVSSAAALDNEKSNGTIIHVKGKHLCPSPLFP | |
| | GPSKPFWVLVVVGGVLACYSLLVTVAFIIFWVRSKRSRLLHSDYM | |
| | NMTPRRPGPTRKHYQPYAPPRDFAAYRSRVKFSRSADAPAYQQG | |
| | QNQLYNELNLGRREEYDVLDKRRGRDPEMGGKPRRKNPQEGLYN | |
| | ELQKDKMAEAYSEIGMKGERRRGKGHDGLYQGLSTATKDTYDA | |
| | LHMQALPPR | |
| 126 | QVQLVESGGGVVQPGRSLRLSCAASGFTFSSYGMHWVRQAPGKG | anti-BCMA |
| | LEWVAVISYDGSNKYYADSVKGRFTISRDNSKNTLYLQMNSLRAE | CAR |
| | DTAVYYCARDGTYLGGLWYFDLWGRGTLVTVSSGSTSGSGKPGS | |
| | GEGSTKGDIVMTQSPLSLPVTPGEPASISCRSSQSLLHSNGYNYLD | |
| | WYLQKPGQSPQLLIYLGSNRASGVPDRFSGSGSGTDFTLKISRVEA | |
| | EDVGVYYCMQGLGLPLTFGGGTKVEIKRAAALDNEKSNGTIIHVK | |
| | GKHLCPSPLFPGPSKPFWVLVVVGGVLACYSLLVTVAFIIFWVRSK | |
| | RSRLLHSDYMNMTPRRPGPTRKHYQPYAPPRDFAAYRSRVKFSRS | |
| | ADAPAYQQGQNQLYNELNLGRREEYDVLDKRRGRDPEMGGKPR | |
| | RKNPQEGLYNELQKDKMAEAYSEIGMKGERRRGKGHDGLYQGL | |
| | STATKDTYDALHMQALPPR | |
| 127 | DIVMTQSPLSLPVTPGEPASISCRSSQSLLHSNGYNYLDWYLQKPG | anti-BCMA |
| | QSPQLLIYLGSNRASGVPDRFSGSGSGTDFTLKISRVEAEDVGVYY | CAR |
| | CMQGLGLPLTFGGGTKVEIKRGSTSGSGKPGSGEGSTKGQVQLVE | |
| | SGGGVVQPGRSLRLSCAASGFTFSSYGMHWVRQAPGKGLEWVAV | |
| | ISYDGSNKYYADSVKGRFTISRDNSKNTLYLQMNSLRAEDTAVYY | |
| | CARDGTYLGGLWYFDLWGRGTLVTVSSAAALDNEKSNGTIIHVK | |
| | GKHLCPSPLFPGPSKPFWVLVVVGGVLACYSLLVTVAFIIFWVRSK | |
| | RSRLLHSDYMNMTPRRPGPTRKHYQPYAPPRDFAAYRSRVKFSRS | |
| | ADAPAYQQGQNQLYNELNLGRREEYDVLDKRRGRDPEMGGKPR | |
| | RKNPQEGLYNELQKDKMAEAYSEIGMKGERRRGKGHDGLYQGL | |
| | STATKDTYDALHMQALPPR | |
| 128 | QVQLVQSGAEVKKPGASVKVSCKASGYTFTSYYMHWVRQAPGQ | anti-BCMA |
| | GLEWMGIINPGGGSTSYAQKFQGRVTMTRDTSTSTVYMELSSLRS | CAR |
| | EDTAVYYCARESWPMDVWGQGTTVTVSSGSTSGSGKPGSGEGST | |
| | KGEIVMTQSPATLSVSPGERATLSCRASQSVSSNLAWYQQKPGQA | |
| | PRLLIYGASTRATGIPARFSGSGSGTEFTLTISSLQSEDFAVYYCQQ | |
| | YAAYPTFGGGTKVEIKRAAALDNEKSNGTIIHVKGKHLCPSPLFPG | |
| | PSKPFWVLVVVGGVLACYSLLVTVAFIIFWVRSKRSRLLHSDYMN | |
| | MTPRRPGPTRKHYQPYAPPRDFAAYRSRVKFSRSADAPAYQQGQ | |
| | NQLYNELNLGRREEYDVLDKRRGRDPEMGGKPRRKNPQEGLYNE | |
| | LQKDKMAEAYSEIGMKGERRRGKGHDGLYQGLSTATKDTYDAL | |
| | HMQALPPR | |
| 129 | EIVMTQSPATLSVSPGERATLSCRASQSVSSNLAWYQQKPGQAPR | anti-BCMA |
| | LLIYGASTRATGIPARFSGSGSGTEFTLTISSLQSEDFAVYYCQQYA | CAR |
| | AYPTFGGGTKVEIKRGSTSGSGKPGSGEGSTKGQVQLVQSGAEVK | |
| | KPGASVKVSCKASGYTFTSYYMHWVRQAPGQGLEWMGIINPGGG | |
| | STSYAQKFQGRVTMTRDTSTSTVYMELSSLRSEDTAVYYCARESW | |
| | PMDVWGQGTTVTVSSAAALDNEKSNGTIIHVKGKHLCPSPLFPGP | |
| | SKPFWVLVVVGGVLACYSLLVTVAFIIFWVRSKRSRLLHSDYMN | |
| | MTPRRPGPTRKHYQPYAPPRDFAAYRSRVKFSRSADAPAYQQGQ | |
| | NQLYNELNLGRREEYDVLDKRRGRDPEMGGKPRRKNPQEGLYNE | |
| | LQKDKMAEAYSEIGMKGERRRGKGHDGLYQGLSTATKDTYDAL | |
| | HMQALPPR | |
| 130 | QLQLQESGPGLVKPSETLSLTCTVSGGSISSSSYYWGWIRQPPGKG | anti-BCMA |
| | LEWIGSISYSGSTYYNPSLKSRVTISVDTSKNQFSLKLSSVTAADTA | CAR |
| | VYYCARGRGYATSLAFDIWGQGTMVTVSSGSTSGSGKPGSGEGST | |
| | KGEIVLTQSPATLSLSPGERATLSCRASQSVSSYLAWYQQKPGQAP | |
| | RLLIYDASNRATGIPARFSGSGSGTDFTLTISSLEPEDFAVYYCQQR | |
| | HVWPPTFGGGTKVEIKRAAALDNEKSNGTIIHVKGKHLCPSPLFPG | |
| | PSKPFWVLVVVGGVLACYSLLVTVAFIIFWVRSKRSRLLHSDYMN | |
| | MTPRRPGPTRKHYQPYAPPRDFAAYRSRVKFSRSADAPAYQQGQ | |
| | NQLYNELNLGRREEYDVLDKRRGRDPEMGGKPRRKNPQEGLYNE | |
| | LQKDKMAEAYSEIGMKGERRRGKGHDGLYQGLSTATKDTYDAL | |
| | HMQALPPR | |
| 131 | EIVLTQSPATLSLSPGERATLSCRASQSVSSYLAWYQQKPGQAPRL | anti-BCMA |
| | LIYDASNRATGIPARFSGSGSGTDFTLTISSLEPEDFAVYYCQQRHV | CAR |
| | WPPTFGGGTKVEIKRGSTSGSGKPGSGEGSTKGQLQLQESGPGLV | |
| | KPSETLSLTCTVSGGSISSSSYYWGWIRQPPGKGLEWIGSISYSGST | |
| | YYNPSLKSRVTISVDTSKNQFSLKLSSVTAADTAVYYCARGRGYA | |
| | TSLAFDIWGQGTMVTVSSAAALDNEKSNGTIIHVKGKHLCPSPLFP | |
| | GPSKPFWVLVVVGGVLACYSLLVTVAFIIFWVRSKRSRLLHSDYM | |
| | NMTPRRPGPTRKHYQPYAPPRDFAAYRSRVKFSRSADAPAYQQG | |
| | QNQLYNELNLGRREEYDVLDKRRGRDPEMGGKPRRKNPQEGLYN | |
| | ELQKDKMAEAYSEIGMKGERRRGKGHDGLYQGLSTATKDTYDA | |
| | LHMQALPPR | |
| 132 | EVQLVESGGGLVQPGGSLRLSCAASGFTFSSYSMNWVRQAPGKG | anti-BCMA |
| | LEWVSTISSSSSTIYYADSVKGRFTISRDNAKNSLYLQMNSLRAED | CAR |
| | TAVYYCARGSQEHLIFDYWGQGTLVTVSSGSTSGSGKPGSGEGST | |
| | KGEIVLTQSPATLSLSPGERATLSCRASQSVSRYLAWYQQKPGQAP | |
| | RLLIYDASNRATGIPARFSGSGSGTDFTLTISSLEPEDFAVYYCQQR | |
| | FYYPWTFGGGTKVEIKRAAALDNEKSNGTIIHVKGKHLCPSPLFPG | |
| | PSKPFWVLVVVGGVLACYSLLVTVAFIIFWVRSKRSRLLHSDYMN | |
| | MTPRRPGPTRKHYQPYAPPRDFAAYRSRVKFSRSADAPAYQQGQ | |
| | NQLYNELNLGRREEYDVLDKRRGRDPEMGGKPRRKNPQEGLYNE | |
| | LQKDKMAEAYSEIGMKGERRRGKGHDGLYQGLSTATKDTYDAL | |
| | HMQALPPR | |
| 133 | EIVLTQSPATLSLSPGERATLSCRASQSVSRYLAWYQQKPGQAPRL | anti-BCMA |
| | LIYDASNRATGIPARFSGSGSGTDFTLTISSLEPEDFAVYYCQQRFY | CAR |
| | YPWTFGGGTKVEIKRGSTSGSGKPGSGEGSTKGEVQLVESGGGLV | |
| | QPGGSLRLSCAASGFTFSSYSMNWVRQAPGKGLEWVSTISSSSSTI | |
| | YYADSVKGRFTISRDNAKNSLYLQMNSLRAEDTAVYYCARGSQE | |
| | HLIFDYWGQGTLVTVSSAAALDNEKSNGTIIHVKGKHLCPSPLFPG | |
| | PSKPFWVLVVVGGVLACYSLLVTVAFIIFWVRSKRSRLLHSDYMN | |
| | MTPRRPGPTRKHYQPYAPPRDFAAYRSRVKFSRSADAPAYQQGQ | |
| | NQLYNELNLGRREEYDVLDKRRGRDPEMGGKPRRKNPQEGLYNE | |
| | LQKDKMAEAYSEIGMKGERRRGKGHDGLYQGLSTATKDTYDAL | |
| | HMQALPPR | |
| 134 | QVQLVESGGGVVQPGRSLRLSCAASGFTFSSYGMHWVRQAPGKG | anti-BCMA |
| | LEWVAVISYDGSNKYYADSVKGRFTISRDNSKNTLYLQMNSLRAE | CAR |
| | DTAVYYCARTDFWSGSPPGLDYWGQGTLVTVSSGSTSGSGKPGS | |
| | GEGSTKGDIQLTQSPSSVSASVGDRVTITCRASQGISSWLAWYQQK | |
| | PGKAPKLLIYGASSLQSGVPSRFSGSGSGTDFTLTISSLQPEDFATY | |
| | YCQQIYTFPFTFGGGTKVEIKRAAALDNEKSNGTIIHVKGKHLCPS | |
| | PLFPGPSKPFWVLVVVGGVLACYSLLVTVAFIIFWVRSKRSRLLHS | |
| | DYMNMTPRRPGPTRKHYQPYAPPRDFAAYRSRVKFSRSADAPAY | |
| | QQGQNQLYNELNLGRREEYDVLDKRRGRDPEMGGKPRRKNPQE | |
| | GLYNELQKDKMAEAYSEIGMKGERRRGKGHDGLYQGLSTATKD | |
| | TYDALHMQALPPR | |
| 135 | DIQLTQSPSSVSASVGDRVTITCRASQGISSWLAWYQQKPGKAPKL | anti-BCMA |
| | LIYGASSLQSGVPSRFSGSGSGTDFTLTISSLQPEDFATYYCQQIYTF | CAR |
| | PFTFGGGTKVEIKRGSTSGSGKPGSGEGSTKGQVQLVESGGGVVQ | |
| | PGRSLRLSCAASGFTFSSYGMHWVRQAPGKGLEWVAVISYDGSN | |
| | KYYADSVKGRFTISRDNSKNTLYLQMNSLRAEDTAVYYCARTDF | |
| | WSGSPPGLDYWGQGTLVTVSSAAALDNEKSNGTIIHVKGKHLCPS | |
| | PLFPGPSKPFWVLVVVGGVLACYSLLVTVAFIIFWVRSKRSRLLHS | |
| | DYMNMTPRRPGPTRKHYQPYAPPRDFAAYRSRVKFSRSADAPAY | |
| | QQGQNQLYNELNLGRREEYDVLDKRRGRDPEMGGKPRRKNPQE | |
| | GLYNELQKDKMAEAYSEIGMKGERRRGKGHDGLYQGLSTATKD | |
| | TYDALHMQALPPR | |
| 136 | QVQLVQSGAEVKKPGSSVKVSCKASGGTFSSYAISWVRQAPGQGL | anti-BCMA |
| | EWMGGIIPIFGTANYAQKFQGRVTITADESTSTAYMELSSLRSEDT | CAR |
| | AVYYCARTPEYSSSIWHYYYGMDVWGQGTTVTVSSGSTSGSGKP | |
| | GSGEGSTKGDIVMTQSPDSLAVSLGERATINCKSSQSVLYSSNNKN | |
| | YLAWYQQKPGQPPKLLIYWASTRESGVPDRFSGSGSGTDFTLTISS | |
| | LQAEDVAVYYCQQFAHTPFTFGGGTKVEIKRAAALDNEKSNGTII | |
| | HVKGKHLCPSPLFPGPSKPFWVLVVVGGVLACYSLLVTVAFIIFW | |
| | VRSKRSRLLHSDYMNMTPRRPGPTRKHYQPYAPPRDFAAYRSRV | |
| | KFSRSADAPAYQQGQNQLYNELNLGRREEYDVLDKRRGRDPEMG | |
| | GKPRRKNPQEGLYNELQKDKMAEAYSEIGMKGERRRGKGHDGL | |
| | YQGLSTATKDTYDALHMQALPPR | |
| 137 | DIVMTQSPDSLAVSLGERATINCKSSQSVLYSSNNKNYLAWYQQK | anti-BCMA |
| | PGQPPKLLIYWASTRESGVPDRFSGSGSGTDFTLTISSLQAEDVAV | CAR |
| | YYCQQFAHTPFTFGGGTKVEIKRGSTSGSGKPGSGEGSTKGQVQL | |
| | VQSGAEVKKPGSSVKVSCKASGGTFSSYAISWVRQAPGQGLEWM | |
| | GGIIPIFGTANYAQKFQGRVTITADESTSTAYMELSSLRSEDTAVYY | |
| | CARTPEYSSSIWHYYYGMDVWGQGTTVTVSSAAALDNEKSNGTII | |
| | HVKGKHLCPSPLFPGPSKPFWVLVVVGGVLACYSLLVTVAFIIFW | |
| | VRSKRSRLLHSDYMNMTPRRPGPTRKHYQPYAPPRDFAAYRSRV | |
| | KFSRSADAPAYQQGQNQLYNELNLGRREEYDVLDKRRGRDPEMG | |
| | GKPRRKNPQEGLYNELQKDKMAEAYSEIGMKGERRRGKGHDGL | |
| | YQGLSTATKDTYDALHMQALPPR | |
| 138 | QVQLVESGGGVVQPGRSLRLSCAASGFTFSSYGMHWVRQAPGKG | anti-BCMA |
| | LEWVAVISYDGSNKYYADSVKGRFTISRDNSKNTLYLQMNSLRAE | CAR |
| | DTAVYYCVKGPLQEPPYDYGMDVWGQGTTVTVSSGSTSGSGKPG | |
| | SGEGSTKGEIVMTQSPATLSVSPGERATLSCRASQSVSSNLAWYQQ | |
| | KPGQAPRLLIYSASTRATGIPARFSGSGSGTEFTLTISSLQSEDFAVY | |
| | YCQQHHVWPLTFGGGTKVEIKRAAALDNEKSNGTIIHVKGKHLCP | |
| | SPLFPGPSKPFWVLVVVGGVLACYSLLVTVAFIIFWVRSKRSRLLH | |
| | SDYMNMTPRRPGPTRKHYQPYAPPRDFAAYRSRVKFSRSADAPA | |
| | YQQGQNQLYNELNLGRREEYDVLDKRRGRDPEMGGKPRRKNPQ | |
| | EGLYNELQKDKMAEAYSEIGMKGERRRGKGHDGLYQGLSTATK | |
| | DTYDALHMQALPPR | |
| 139 | EIVMTQSPATLSVSPGERATLSCRASQSVSSNLAWYQQKPGQAPR | anti-BCMA |
| | LLIYSASTRATGIPARFSGSGSGTEFTLTISSLQSEDFAVYYCQQHH | CAR |
| | VWPLTFGGGTKVEIKRGSTSGSGKPGSGEGSTKGQVQLVESGGGV | |
| | VQPGRSLRLSCAASGFTFSSYGMHWVRQAPGKGLEWVAVISYDG | |
| | SNKYYADSVKGRFTISRDNSKNTLYLQMNSLRAEDTAVYYCVKG | |
| | PLQEPPYDYGMDVWGQGTTVTVSSAAALDNEKSNGTIIHVKGKH | |
| | LCPSPLFPGPSKPFWVLVVVGGVLACYSLLVTVAFIIFWVRSKRSR | |
| | LLHSDYMNMTPRRPGPTRKHYQPYAPPRDFAAYRSRVKFSRSAD | |
| | APAYQQGQNQLYNELNLGRREEYDVLDKRRGRDPEMGGKPRRK | |
| | NPQEGLYNELQKDKMAEAYSEIGMKGERRRGKGHDGLYQGLSTA | |
| | TKDTYDALHMQALPPR | |
| 140 | QSALTQPASVSASPGQSIAISCTGTSSDVGWYQQHPGKAPKLMIYE | anti-BCMA |
| | DSKRPSGVSNRFSGSKSGNTASLTISGLQAEDEADYYCSSNTRSST | CAR |
| | LVFGGGTKLTVLGSRGGGGSGGGGSGGGGSLEMAEVQLVQSGAE | |
| | MKKPGASLKLSCKASGYTFIDYYVYWMRQAPGQGLESMGWINPN | |
| | SGGTNYAQKFQGRVTMTRDTSISTAYMELSRLRSDDTAMYYCAR | |
| | SQRDGYMDYWGQGTLVTVSSAAAIEVMYPPPYLDNEKSNGTIIHV | |
| | KGKHLCPSPLFPGPSKPFWVLVVVGGVLACYSLLVTVAFIIFWVRS | |
| | KRSRLLHSDYMNMTPRRPGPTRKHYQPYAPPRDFAAYRSRVKFSR | |
| | SADAPAYQQGQNQLYNELNLGRREEYDVLDKRRGRDPEMGGKP | |
| | RRKNPQEGLYNELQKDKMAEAYSEIGMKGERRRGKGHDGLYQG | |
| | LSTATKDTYDALHMQALPPR | |
| 141 | QSVLTQPPSVSGAPGQRVTISCTGSSSNIGAGFDVHWYQQLPGTAP | anti-BCMA |
| | KLLIYGNSNRPSGVPDRFSGSKSGTSASLAITGLQAEDEADYYCQS | CAR |
| | YDSSLSGYVFGTGTKVTVLGSRGGGGSGGGGSGGGGSLEMAQVQ | |
| | LVQSGAEVKKPGASVKVSCKASGYTFTDYYMHWVRQAPGQRLE | |
| | WMGWINPNSGGTNYAQKFQDRITVTRDTSSNTGYMELTRLRSDD | |
| | TAVYYCARSPYSGVLDKWGQGTLVTVSSAAAIEVMYPPPYLDNE | |
| | KSNGTIIHVKGKHLCPSPLFPGPSKPFWVLVVVGGVLACYSLLVTV | |
| | AFIIFWVRSKRSRLLHSDYMNMTPRRPGPTRKHYQPYAPPRDFAA | |
| | YRSRVKFSRSADAPAYQQGQNQLYNELNLGRREEYDVLDKRRGR | |
| | DPEMGGKPRRKNPQEGLYNELQKDKMAEAYSEIGMKGERRRGK | |
| | GHDGLYQGLSTATKDTYDALHMQALPPR | |
| 142 | SYELTQPPSASGTPGQRVTMSCSGTSSNIGSHSVNWYQQLPGTAPK | anti-BCMA |
| | LLIYTNNQRPSGVPDRFSGSKSGTSASLAISGLQSEDEADYYCAAW | CAR |
| | DGSLNGLVFGGGTKLTVLGSRGGGGSGGGGSGGGGSLEMAEVQL | |
| | VQSGAEVKKPGESLKISCKGSGYSFTSYWIGWVRQMPGKGLEWM | |
| | GIIYPGDSDTRYSPSFQGHVTISADKSISTAYLQWSSLKASDTAMY | |
| | YCARYSGSFDNWGQGTLVTVSSAAAIEVMYPPPYLDNEKSNGTII | |
| | HVKGKHLCPSPLFPGPSKPFWVLVVVGGVLACYSLLVTVAFIIFW | |
| | VRSKRSRLLHSDYMNMTPRRPGPTRKHYQPYAPPRDFAAYRSRV | |
| | KFSRSADAPAYQQGQNQLYNELNLGRREEYDVLDKRRGRDPEMG | |
| | GKPRRKNPQEGLYNELQKDKMAEAYSEIGMKGERRRGKGHDGL | |
| | YQGLSTATKDTYDALHMQALPPR | |
| 143 | LPVLTQPPSASGTPGQRVTISCSGRSSNIGSNSVNWYRQLPGAAPK | anti-BCMA |
| | LLIYSNNQRPPGVPVRFSGSKSGTSASLAISGLQSEDEATYYCATW | CAR |
| | DDNLNVHYVFGTGTKVTVLGSRGGGGSGGGGSGGGGSLEMAQV | |
| | QLVQSGAEVKKPGSSVKVSCKASGGTFSSYAISWVRQAPGQGLE | |
| | WMGRIIPILGIANYAQKFQGRVTITADKSTSTAYMELSSLRSEDTA | |
| | VYYCARGGYYSHDMWSEDWGQGTLVTVSSAAAIEVMYPPPYLD | |
| | NEKSNGTIIHVKGKHLCPSPLFPGPSKPFWVLVVVGGVLACYSLLV | |
| | TVAFIIFWVRSKRSRLLHSDYMNMTPRRPGPTRKHYQPYAPPRDF | |
| | AAYRSRVKFSRSADAPAYQQGQNQLYNELNLGRREEYDVLDKRR | |
| | GRDPEMGGKPRRKNPQEGLYNELQKDKMAEAYSEIGMKGERRR | |
| | GKGHDGLYQGLSTATKDTYDALHMQALPPR | |
| 144 | QAVLTQPPSASGTPGQRVTISCSGSSSNIGSNYVFWYQQLPGTAPK | anti-BCMA |
| | LLIYSNNQRPSGVPDRFSGSKSGTSASLAISGLRSEDEADYYCAAW | CAR |
| | DDSLSASYVFGTGTKVTVLGSRGGGGSGGGGSGGGGSLEMAQVQ | |
| | LVQSGAEVKKPGSSVKVSCKASGGTFSSYAISWVRQAPGQGLEW | |
| | MGRIIPILGTANYAQKFQGRVTITADESTSTAYMELSSLRSEDTAV | |
| | YYCARSGYGSYRWEDSWGQGTLVTVSSAAAIEVMYPPPYLDNEK | |
| | SNGTIIHVKGKHLCPSPLFPGPSKPFWVLVVVGGVLACYSLLVTVA | |
| | FIIFWVRSKRSRLLHSDYMNMTPRRPGPTRKHYQPYAPPRDFAAY | |
| | RSRVKFSRSADAPAYQQGQNQLYNELNLGRREEYDVLDKRRGRD | |
| | PEMGGKPRRKNPQEGLYNELQKDKMAEAYSEIGMKGERRRGKG | |
| | HDGLYQGLSTATKDTYDALHMQALPPR | |
| 145 | LPVLTQPPSASGTPGQRVTISCSGRSSNIGSNSVNWYRQLPGAAPK | anti-BCMA |
| | LLIYSNNQRPPGVPVRFSGSKSGTSASLAISGLQSEDEATYYCATW | CAR |
| | DDNLNVHYVFGTGTKVTVLGSRGGGGSGGGGSGGGGSLEMAQV | |
| | QLVQSGAEVKKPGSSVKVSCKASGGTFSSYAISWVRQAPGQGLE | |
| | WMGRIIPILGIANYAQKFQGRVTITADKSTSTAYMELSSLRSEDTA | |
| | VYYCARGGYYSHDMWSEDWGQGTLVTVSSAAAPTTTPAPRPPTP | |
| | APTIASQPLSLRPEACRPAAGGAVHTRGLDFACDIYIWAPLAGTCG | |
| | VLLLSLVITLYCNKRGRKKLLYIFKQPFMRPVQTTQEEDGCSCRFP | |
| | EEEEGGCELRVKFSRSAEPPAYQQGQNQLYNELNLGRREEYDVLD | |
| | KRRGRDPEMGGKPRRKNPQEGLYNELQKDKMAEAYSEIGMKGE | |
| | RRRGKGHDGLYQGLSTATKDTYDALHMQALPPR | |
| 146 | SYELTQPPSASGTPGQRVTMSCSGTSSNIGSHSVNWYQQLPGTAPK | anti-BCMA |
| | LLIYTNNQRPSGVPDRFSGSKSGTSASLAISGLQSEDEADYYCAAW | CAR |
| | DGSLNGLVFGGGTKLTVLGSRGGGGSGGGGSGGGGSLEMAEVQL | |
| | VQSGAEVKKPGESLKISCKGSGYSFTSYWIGWVRQMPGKGLEWM | |
| | GIIYPGDSDTRYSPSFQGHVTISADKSISTAYLQWSSLKASDTAMY | |
| | YCARYSGSFDNWGQGTLVTVSSAAAPTTTPAPRPPTPAPTIASQPL | |
| | SLRPEACRPAAGGAVHTRGLDFACDIYIWAPLAGTCGVLLLSLVIT | |
| | LYCNKRGRKKLLYIFKQPFMRPVQTTQEEDGCSCRFPEEEEGGCEL | |
| | RVKFSRSAEPPAYQQGQNQLYNELNLGRREEYDVLDKRRGRDPE | |
| | MGGKPRRKNPQEGLYNELQKDKMAEAYSEIGMKGERRRGKGHD | |
| | GLYQGLSTATKDTYDALHMQALPPR | |
| 147 | QAVLTQPPSASGTPGQRVTISCSGSSSNIGSNYVFWYQQLPGTAPK | anti-BCMA |
| | LLIYSNNQRPSGVPDRFSGSKSGTSASLAISGLRSEDEADYYCAAW | CAR |
| | DDSLSASYVFGTGTKVTVLGSRGGGGSGGGGSGGGGSLEMAQVQ | |
| | LVQSGAEVKKPGSSVKVSCKASGGTFSSYAISWVRQAPGQGLEW | |
| | MGRIIPILGTANYAQKFQGRVTITADESTSTAYMELSSLRSEDTAV | |
| | YYCARSGYGSYRWEDSWGQGTLVTVSSAAAPTTTPAPRPPTPAPT | |
| | IASQPLSLRPEACRPAAGGAVHTRGLDFACDIYIWAPLAGTCGVLL | |
| | LSLVITLYCNKRGRKKLLYIFKQPFMRPVQTTQEEDGCSCRFPEEE | |
| | EGGCELRVKFSRSAEPPAYQQGQNQLYNELNLGRREEYDVLDKR | |
| | RGRDPEMGGKPRRKNPQEGLYNELQKDKMAEAYSEIGMKGERRR | |
| | GKGHDGLYQGLSTATKDTYDALHMQALPPR | |
| 148 | QSVLTQPPSVSGAPGQRVTISCTGSSSNIGAGFDVHWYQQLPGTAP | anti-BCMA |
| | KLLIYGNSNRPSGVPDRFSGSKSGTSASLAITGLQAEDEADYYCQS | CAR |
| | YDSSLSGYVFGTGTKVTVLGSRGGGGSGGGGSGGGGSLEMAQVQ | |
| | LVQSGAEVKKPGASVKVSCKASGYTFTDYYMHWVRQAPGQRLE | |
| | WMGWINPNSGGTNYAQKFQDRITVTRDTSSNTGYMELTRLRSDD | |
| | TAVYYCARSPYSGVLDKWGQGTLVTVSSAAAPTTTPAPRPPTPAP | |
| | TIASQPLSLRPEACRPAAGGAVHTRGLDFACDIYIWAPLAGTCGVL | |
| | LLSLVITLYCNKRGRKKLLYIFKQPFMRPVQTTQEEDGCSCRFPEE | |
| | EEGGCELRVKFSRSAEPPAYQQGQNQLYNELNLGRREEYDVLDK | |
| | RRGRDPEMGGKPRRKNPQEGLYNELQKDKMAEAYSEIGMKGERR | |
| | RGKGHDGLYQGLSTATKDTYDALHMQALPPR | |
| 149 | QSALTQPASVSASPGQSIAISCTGTSSDVGWYQQHPGKAPKLMIYE | anti-BCMA |
| | DSKRPSGVSNRFSGSKSGNTASLTISGLQAEDEADYYCSSNTRSST | CAR |
| | LVFGGGTKLTVLGSRGGGGSGGGGSGGGGSLEMAEVQLVQSGAE | |
| | MKKPGASLKLSCKASGYTFIDYYVYWMRQAPGQGLESMGWINPN | |
| | SGGTNYAQKFQGRVTMTRDTSISTAYMELSRLRSDDTAMYYCAR | |
| | SQRDGYMDYWGQGTLVTVSSAAAPTTTPAPRPPTPAPTIASQPLSL | |
| | RPEACRPAAGGAVHTRGLDFACDIYIWAPLAGTCGVLLLSLVITLY | |
| | CNKRGRKKLLYIFKQPFMRPVQTTQEEDGCSCRFPEEEEGGCELR | |
| | VKFSRSAEPPAYQQGQNQLYNELNLGRREEYDVLDKRRGRDPEM | |
| | GGKPRRKNPQEGLYNELQKDKMAEAYSEIGMKGERRRGKGHDG | |
| | LYQGLSTATKDTYDALHMQALPPR | |
| 150 | DIVLTQSPASLAVSLGERATINCRASESVSVIGAHLIHWYQQKPGQ | anti-BCMA |
| | PPKLLIYLASNLETGVPARFSGSGSGTDFTLTISSLQAEDAAIYYCL | CAR |
| | QSRIFPRTFGQGTKLEIKGSTSGSGKPGSGEGSTKGQVQLVQSGSEL | |
| | KKPGASVKVSCKASGYTFTDYSINWVRQAPGQGLEWMGWINTET | |
| | REPAYAYDFRGRFVFSLDTSVSTAYLQISSLKAEDTAVYYCARDY | |
| | SYAMDYWGQGTLVTVSSAAATTTPAPRPPTPAPTIASQPLSLRPEA | |
| | CRPAAGGAVHTRGLDFACDIYIWAPLAGTCGVLLLSLVITLYCKR | |
| | GRKKLLYIFKQPFMRPVQTTQEEDGCSCRFPEEEEGGCELRVKFSR | |
| | SADAPAYQQGQNQLYNELNLGRREEYDVLDKRRGRDPEMGGKP | |
| | RRKNPQEGLYNELQKDKMAEAYSEIGMKGERRRGKGHDGLYQG | |
| | LSTATKDTYDALHMQALPPR | |
| 151 | DIVLTQSPASLAVSLGERATINCRASESVSVIGAHLIHWYQQKPGQ | anti-BCMA |
| | PPKLLIYLASNLETGVPARFSGSGSGTDFTLTISSLQAEDAAIYYCL | CAR |
| | QSRIFPRTFGQGTKLEIKGSTSGSGKPGSGEGSTKGQVQLVQSGSEL | |
| | KKPGASVKVSCKASGYTFTDYSINWVRQAPGQGLEWMGWINTET | |
| | REPAYAYDFRGRFVFSLDTSVSTAYLQISSLKAEDTAVYYCARDY | |
| | SYAMDYWGQGTLVTVSSAAADTGLYICKVELMYPPPYYLGIGNG | |
| | TQIYVIDPEPCPDSDFLLWILAAVSSGLFFYSFLLTAVSKRGRKKLL | |
| | YIFKQPFMRPVQTTQEEDGCSCRFPEEEEGGCELRVKFSRSADAPA | |
| | YQQGQNQLYNELNLGRREEYDVLDKRRGRDPEMGGKPRRKNPQ | |
| | EGLYNELQKDKMAEAYSEIGMKGERRRGKGHDGLYQGLSTATK | |
| | DTYDALHMQALPPR | |
| 152 | DIVLTQSPASLAVSLGERATINCRASESVSVIGAHLIHWYQQKPGQ | anti-BCMA |
| | PPKLLIYLASNLETGVPARFSGSGSGTDFTLTISSLQAEDAAIYYCL | CAR |
| | QSRIFPRTFGQGTKLEIKGSTSGSGKPGSGEGSTKGQVQLVQSGSEL | |
| | KKPGASVKVSCKASGYTFTDYSINWVRQAPGQGLEWMGWINTET | |
| | REPAYAYDFRGRFVFSLDTSVSTAYLQISSLKAEDTAVYYCARDY | |
| | SYAMDYWGQGTLVTVSSAAAQIKESLRAELRVTERRAEVPTAHPS | |
| | PSPRPAGQFQTLVVGVVGGLLGSLVLLVWVLAVICSKRGRKKLLY | |
| | IFKQPFMRPVQTTQEEDGCSCRFPEEEEGGCELRVKFSRSADAPAY | |
| | QQGQNQLYNELNLGRREEYDVLDKRRGRDPEMGGKPRRKNPQE | |
| | GLYNELQKDKMAEAYSEIGMKGERRRGKGHDGLYQGLSTATKD | |
| | TYDALHMQALPPR | |
| 153 | EVQLVESGGGLVKPGGSLRLSCAASGFTFSDYYMSWIRQAPGKGL | anti-BCMA |
| | EWVSYISSSGSTIYYADSVKGRFTISRDNAKNSLYLQMNSLRAEDT | CAR |
| | AVYYCAKVDGDYTEDYWGQGTLVTVSSGGGGSGGGGSGGGGSQ | |
| | SALTQPASVSGSPGQSITISCTGSSSDVGKYNLVSWYQQPPGKAPK | |
| | LIIYDVNKRPSGVSNRFSGSKSGNTATLTISGLQGDDEADYYCSSY | |
| | GGSRSYVFGTGTKVTVLESKYGPPCPPCPAPPVAGPSVFLFPPKPK | |
| | DTLMISRTPEVTCVVVDVSQEDPEVQFNWYVDGVEVHNAKTKPR | |
| | EEQFQSTYRVVSVLTVLHQDWLNGKEYKCKVSNKGLPSSIEKTIS | |
| | KAKGQPREPQVYTLPPSQEEMTKNQVSLTCLVKGFYPSDIAVEWE | |
| | SNGQPENNYKTTPPVLDSDGSFFLYSRLTVDKSRWQEGNVFSCSV | |
| | MHEALHNHYTQKSLSLSLGKMFWVLVVVGGVLACYSLLVTVAFI | |
| | IFWVKRGRKKLLYIFKQPFMRPVQTTQEEDGCSCRFPEEEEGGCEL | |
| | RVKFSRSADAPAYQQGQNQLYNELNLGRREEYDVLDKRRGRDPE | |
| | MGGKPRRKNPQEGLYNELQKDKMAEAYSEIGMKGERRRGKGHD | |
| | GLYQGLSTATKDTYDALHMQALPPR | |
| 154 | EVQLVQSGGGLVQPGRSLRLSCTASGFTFGDYAMSWFKQAPGKG | anti-BCMA |
| | LEWVGFIRSKAYGGTTEYAASVKGRFTISRDDSKSIAYLQMNSLKT | CAR |
| | EDTAVYYCAAWSAPTDYWGQGTLVTVSSGGGGSGGGGSGGGGS | |
| | DIQMTQSPAFLSASVGDRVTVTCRASQGISNYLAWYQQKPGNAPR | |
| | LLIYSASTLQSGVPSRFRGTGYGTEFSLTIDSLQPEDFATYYCQQSY | |
| | TSRQTFGPGTRLDIKESKYGPPCPPCPAPPVAGPSVFLFPPKPKDTL | |
| | MISRTPEVTCVVVDVSQEDPEVQFNWYVDGVEVHNAKTKPREEQ | |
| | FQSTYRVVSVLTVLHQDWLNGKEYKCKVSNKGLPSSIEKTISKAK | |
| | GQPREPQVYTLPPSQEEMTKNQVSLTCLVKGFYPSDIAVEWESNG | |
| | QPENNYKTTPPVLDSDGSFFLYSRLTVDKSRWQEGNVFSCSVMHE | |
| | ALHNHYTQKSLSLSLGKMFWVLVVVGGVLACYSLLVTVAFIIFW | |
| | VKRGRKKLLYIFKQPFMRPVQTTQEEDGCSCRFPEEEEGGCELRV | |
| | KFSRSADAPAYQQGQNQLYNELNLGRREEYDVLDKRRGRDPEMG | |
| | GKPRRKNPQEGLYNELQKDKMAEAYSEIGMKGERRRGKGHDGL | |
| | YQGLSTATKDTYDALHMQALPPR | |
| 155 | EVOLVESGGGLVKPGGSLRLSCAASGFTFSDYYMSWIRQAPGKGL | anti-BCMA |
| | EWVSYISSSGSTIYYADSVKGRFTISRDNAKNSLYLQMNSLRAEDT | CAR |
| | AVYYCAKVDGPPSFDIWGQGTMVTVSSGGGGSGGGGSGGGGSSY | |
| | VLTQPPSVSVAPGQTARITCGANNIGSKSVHWYQQKPGQAPMLVV | |
| | YDDDDRPSGIPERFSGSNSGNTATLTISGVEAGDEADYFCHLWDRS | |
| | RDHYVFGTGTKLTVLESKYGPPCPPCPAPPVAGPSVFLFPPKPKDT | |
| | LMISRTPEVTCVVVDVSQEDPEVQFNWYVDGVEVHNAKTKPREE | |
| | QFQSTYRVVSVLTVLHQDWLNGKEYKCKVSNKGLPSSIEKTISKA | |
| | KGQPREPQVYTLPPSQEEMTKNQVSLTCLVKGFYPSDIAVEWESN | |
| | GQPENNYKTTPPVLDSDGSFFLYSRLTVDKSRWQEGNVFSCSVMH | |
| | EALHNHYTQKSLSLSLGKMFWVLVVVGGVLACYSLLVTVAFIIFW | |
| | VKRGRKKLLYIFKQPFMRPVQTTQEEDGCSCRFPEEEEGGCELRV | |
| | KFSRSADAPAYQQGQNQLYNELNLGRREEYDVLDKRRGRDPEMG | |
| | GKPRRKNPQEGLYNELQKDKMAEAYSEIGMKGERRRGKGHDGL | |
| | YQGLSTATKDTYDALHMQALPPR | |
| 156 | SYELTQPPSASGTPGQRVTMSCSGTSSNIGSHSVNWYQQLPGTAPK | anti-BCMA |
| | LLIYTNNQRPSGVPDRFSGSKSGTSASLAISGLQSEDEADYYCAAW | CAR |
| | DGSLNGLVFGGGTKLTVLGSRGGGGSGGGGSGGGGSLEMAEVQL | |
| | VQSGAEVKKPGESLKISCKGSGYSFTSYWIGWVRQMPGKGLEWM | |
| | GIIYPGDSDTRYSPSFQGHVTISADKSISTAYLQWSSLKASDTAMY | |
| | YCARYSGSFDNWGQGTLVTVSSESKYGPPCPPCPAPPVAGPSVFLF | |
| | PPKPKDTLMISRTPEVTCVVVDVSQEDPEVQFNWYVDGVEVHNA | |
| | KTKPREEQFQSTYRVVSVLTVLHQDWLNGKEYKCKVSNKGLPSSI | |
| | EKTISKAKGQPREPQVYTLPPSQEEMTKNQVSLTCLVKGFYPSDIA | |
| | VEWESNGQPENNYKTTPPVLDSDGSFFLYSRLTVDKSRWQEGNVF | |
| | SCSVMHEALHNHYTQKSLSLSLGKMFWVLVVVGGVLACYSLLVT | |
| | VAFIIFWVKRGRKKLLYIFKQPFMRPVQTTQEEDGCSCRFPEEEEG | |
| | GCELRVKFSRSADAPAYQQGQNQLYNELNLGRREEYDVLDKRRG | |
| | RDPEMGGKPRRKNPQEGLYNELQKDKMAEAYSEIGMKGERRRG | |
| | KGHDGLYQGLSTATKDTYDALHMQALPPR | |
| 157 | QSALTQPASVSASPGQSIAISCTGTSSDVGWYQQHPGKAPKLMIYE | anti-BCMA |
| | DSKRPSGVSNRFSGSKSGNTASLTISGLQAEDEADYYCSSNTRSST | CAR |
| | LVFGGGTKLTVLGSRGGGGSGGGGSGGGGSLEMAEVQLVQSGAE | |
| | MKKPGASLKLSCKASGYTFIDYYVYWMRQAPGQGLESMGWINPN | |
| | SGGTNYAQKFQGRVTMTRDTSISTAYMELSRLRSDDTAMYYCAR | |
| | SQRDGYMDYWGQGTLVTVSSESKYGPPCPPCPAPPVAGPSVFLFP | |
| | PKPKDTLMISRTPEVTCVVVDVSQEDPEVQFNWYVDGVEVHNAK | |
| | TKPREEQFQSTYRVVSVLTVLHQDWLNGKEYKCKVSNKGLPSSIE | |
| | KTISKAKGQPREPQVYTLPPSQEEMTKNQVSLTCLVKGFYPSDIAV | |
| | EWESNGQPENNYKTTPPVLDSDGSFFLYSRLTVDKSRWQEGNVFS | |
| | CSVMHEALHNHYTQKSLSLSLGKMFWVLVVVGGVLACYSLLVT | |
| | VAFIIFWVKRGRKKLLYIFKQPFMRPVQTTQEEDGCSCRFPEEEEG | |
| | GCELRVKFSRSADAPAYQQGQNQLYNELNLGRREEYDVLDKRRG | |
| | RDPEMGGKPRRKNPQEGLYNELQKDKMAEAYSEIGMKGERRRG | |
| | KGHDGLYQGLSTATKDTYDALHMQALPPR | |
| 158 | QSALTQPASVSASPGQSIAISCTGTSSDVGWYQQHPGKAPKLMIYE | anti-BCMA |
| | DSKRPSGVSNRFSGSKSGNTASLTISGLQAEDEADYYCSSNTRSST | CAR |
| | LVFGGGTKLTVLGSRGGGGSGGGGSGGGGSLEMAEVQLVQSGAE | |
| | MKKPGASLKLSCKASGYTFIDYYVYWMRQAPGQGLESMGWINPN | |
| | SGGTNYAQKFQGRVTMTRDTSISTAYMELSRLRSDDTAMYYCAR | |
| | SQRDGYMDYWGQGTLVTVSSESKYGPPCPPCPAPPVAGPSVFLFP | |
| | PKPKDTLMISRTPEVTCVVVDVSQEDPEVQFNWYVDGVEVHNAK | |
| | TKPREEQFQSTYRVVSVLTVLHQDWLNGKEYKCKVSNKGLPSSIE | |
| | KTISKAKGQPREPQVYTLPPSQEEMTKNQVSLTCLVKGFYPSDIAV | |
| | EWESNGQPENNYKTTPPVLDSDGSFFLYSRLTVDKSRWQEGNVFS | |
| | CSVMHEALHNHYTQKSLSLSLGKMFWVLVVVGGVLACYSLLVT | |
| | VAFIIFWVRSKRSRLLHSDYMNMTPRRPGPTRKHYQPYAPPRDFA | |
| | AYRSRVKFSRSADAPAYQQGQNQLYNELNLGRREEYDVLDKRRG | |
| | RDPEMGGKPRRKNPQEGLYNELQKDKMAEAYSEIGMKGERRRG | |
| | KGHDGLYQGLSTATKDTYDALHMQALPPR | |
| 159 | EVQLVESGGGLVQPGGSLRLSCAVSGFALSNHGMSWVRRAPGKG | anti-BCMA |
| | LEWVSGIVYSGSTYYAASVKGRFTISRDNSRNTLYLQMNSLRPED | CAR |
| | TAIYYCSAHGGESDVWGQGTTVTVSSASGGGGSGGRASGGGGSDI | |
| | QLTQSPSSLSASVGDRVTITCRASQSISSYLNWYQQKPGKAPKLLIY | |
| | AASSLQSGVPSRFSGSGSGTDFTLTISSLQPEDFATYYCQQSYSTPY | |
| | TFGQGTKVEIKTTTPAPRPPTPAPTIASQPLSLRPEACRPAAGGAVH | |
| | TRGLDFACDIYIWAPLAGTCGVLLLSLVITLYCKRGRKKLLYIFKQ | |
| | PFMRPVQTTQEEDGCSCRFPEEEEGGCELRVKFSRSADAPAYKQG | |
| | QNQLYNELNLGRREEYDVLDKRRGRDPEMGGKPRRKNPQEGLYN | |
| | ELQKDKMAEAYSEIGMKGERRRGKGHDGLYQGLSTATKDTYDA | |
| | LHMQALPPR | |
| 160 | QVQLVESGGGLVQPGRSLRLSCAASGFTFSNYAMSWVRQAPGKG | anti-BCMA |
| | LGWVSGISRSGENTYYADSVKGRFTISRDNSKNTLYLQMNSLRDE | CAR |
| | DTAVYYCARSPAHYYGGMDVWGQGTTVTVSSASGGGGSGGRAS | |
| | GGGGSDIVLTQSPGTLSLSPGERATLSCRASQSISSSFLAWYQQKPG | |
| | QAPRLLIYGASRRATGIPDRFSGSGSGTDFTLTISRLEPEDSAVYYC | |
| | QQYHSSPSWTFGQGTKLEIKTTTPAPRPPTPAPTIASQPLSLRPEAC | |
| | RPAAGGAVHTRGLDFACDIYIWAPLAGTCGVLLLSLVITLYCKRG | |
| | RKKLLYIFKQPFMRPVQTTQEEDGCSCRFPEEEEGGCELRVKFSRS | |
| | ADAPAYKQGQNQLYNELNLGRREEYDVLDKRRGRDPEMGGKPR | |
| | RKNPQEGLYNELQKDKMAEAYSEIGMKGERRRGKGHDGLYQGL | |
| | STATKDTYDALHMQALPPR | |
| 161 | QVQLVESGGGLVQPGGSLRLSCAVSGFALSNHGMSWVRRAPGKG | anti-BCMA |
| | LEWVSGIVYSGSTYYAASVKGRFTISRDNSRNTLYLQMNSLRPED | CAR |
| | TAIYYCSAHGGESDVWGQGTTVTVSSASGGGGSGGRASGGGGSDI | |
| | RLTQSPSPLSASVGDRVTITCQASEDINKFLNWYHQTPGKAPKLLI | |
| | YDASTLQTGVPSRFSGSGSGTDFTLTINSLQPEDIGTYYCQQYESLP | |
| | LTFGGGTKVEIKTTTPAPRPPTPAPTIASQPLSLRPEACRPAAGGAV | |
| | HTRGLDFACDIYIWAPLAGTCGVLLLSLVITLYCKRGRKKLLYIFK | |
| | QPFMRPVQTTQEEDGCSCRFPEEEEGGCELRVKFSRSADAPAYKQ | |
| | GQNQLYNELNLGRREEYDVLDKRRGRDPEMGGKPRRKNPQEGLY | |
| | NELQKDKMAEAYSEIGMKGERRRGKGHDGLYQGLSTATKDTYD | |
| | ALHMQALPPR | |
| 162 | EVQLVESGGGLVQPGGSLRLSCAVSGFALSNHGMSWVRRAPGKG | anti-BCMA |
| | LEWVSGIVYSGSTYYAASVKGRFTISRDNSRNTLYLQMNSLRPED | CAR |
| | TAIYYCSAHGGESDVWGQGTTVTVSSASGGGGSGGRASGGGGSEI | |
| | VLTQSPGTLSLSPGERATLSCRASQSIGSSSLAWYQQKPGQAPRLL | |
| | MYGASSRASGIPDRFSGSGSGTDFTLTISRLEPEDFAVYYCQQYAG | |
| | SPPFTFGQGTKVEIKTTTPAPRPPTPAPTIASQPLSLRPEACRPAAGG | |
| | AVHTRGLDFACDIYIWAPLAGTCGVLLLSLVITLYCKRGRKKLLYI | |
| | FKQPFMRPVQTTQEEDGCSCRFPEEEEGGCELRVKFSRSADAPAY | |
| | KQGQNQLYNELNLGRREEYDVLDKRRGRDPEMGGKPRRKNPQE | |
| | GLYNELQKDKMAEAYSEIGMKGERRRGKGHDGLYQGLSTATKD | |
| | TYDALHMQALPPR | |
| 163 | QIQLVQSGPDLKKPGETVKLSCKASGYTFTNFGMNWVKQAPGKG | anti-BCMA |
| | FKWMAWINTYTGESYFADDFKGRFAFSVETSATTAYLQINNLKTE | CAR |
| | DTATYFCARGEIYYGYDGGFAYWGQGTLVTVSAGGGGSGGGGS | |
| | GGGGSDVVMTQSHRFMSTSVGDRVSITCRASQDVNTAVSWYQQK | |
| | PGQSPKLLIFSASYRYTGVPDRFTGSGSGADFTLTISSVQAEDLAVY | |
| | YCQQHYSTPWTFGGGTKLDIKTTTPAPRPPTPAPTIASQPLSLRPEA | |
| | CRPAAGGAVHTRGLDFACDIYIWAPLAGTCGVLLLSLVITLYCKR | |
| | GRKKLLYIFKQPFMRPVQTTQEEDGCSCRFPEEEEGGCELRVKFSR | |
| | SADAPAYKQGQNQLYNELNLGRREEYDVLDKRRGRDPEMGGKP | |
| | RRKNPQEGLYNELQKDKMAEAYSEIGMKGERRRGKGHDGLYQG | |
| | LSTATKDTYDALHMQALPPR | |
| 164 | QIQLVQSGPELKKPGETVKISCKASGYTFTDYSINWVKRAPGKGLK | anti-BCMA |
| | WMGWINTETREPAYAYDFRGRFAFSLETSASTAYLQINNLKYEDT | CAR |
| | ATYFCALDYSYAMDYWGQGTSVTVSSGGGGSGGGGSGGGGSQIQ | |
| | LVQSGPELKKPGETVKISCKASGYTFTDYSINWVKRAPGKGLKW | |
| | MGWINTETREPAYAYDFRGRFAFSLETSASTAYLQINNLKYEDTA | |
| | TYFCALDYSYAMDYWGQGTSVTVSSTTTPAPRPPTPAPTIASQPLS | |
| | LRPEACRPAAGGAVHTRGLDFACDIYIWAPLAGTCGVLLLSLVITL | |
| | YCKRGRKKLLYIFKQPFMRPVQTTQEEDGCCRFPEEEEGGCELR | |
| | VKFSRSADAPAYKQGQNQLYNELNLGRREEYDVLDKRRGRDPEM | |
| | GGKPRRKNPQEGLYNELQKDKMAEAYSEIGMKGERRRGKGHDG | |
| | LYQGLSTATKDTYDALHMQALPPR | |
| 165 | QIQLVQSGPELKKPGETVKISCKASGYTFTDYSINWVKRAPGKGLK | anti-BCMA |
| | WMGWINTETREPAYAYDFRGRFAFSLETSASTAYLQINNLKYEDT | CAR |
| | ATYFCALDYSYAMDYWGQGTSVTVSSGGGGSGGGGSGGGGSDIV | |
| | LTQSPASLAMSLGKRATISCRASESVSVIGAHLIHWYQQKPGQPPK | |
| | LLIYLASNLETGVPARFSGSGSGTDFTLTIDPVEEDDVAIYSCLQSRI | |
| | FPRTFGGGTKLEIKTTTPAPRPPTPAPTIASQPLSLRPEACRPAAGGA | |
| | VHTRGLDFACDIYIWAPLAGTCGVLLLSLVITLYCKRGRKKLLYIF | |
| | KQPFMRPVQTTQEEDGCSCRFPEEEEGGCELRVKFSRSADAPAYK | |
| | QGQNQLYNELNLGRREEYDVLDKRRGRDPEMGGKPRRKNPQEGL | |
| | YNELQKDKMAEAYSEIGMKGERRRGKGHDGLYQGLSTATKDTY | |
| | DALHMQALPPR | |
| 166 | QIQLVQSGPELKKPGETVKISCKASGYTFRHYSMNWVKQAPGKGL | anti-BCMA |
| | KWMGRINTESGVPIYADDFKGRFAFSVETSASTAYLVINNLKDED | CAR |
| | TASYFCSNDYLYSLDFWGQGTALTVSSGGGGSGGGGSGGGGSDIV | |
| | LTQSPPSLAMSLGKRATISCRASESVTILGSHLIYWYQQKPGQPPTL | |
| | LIQLASNVQTGVPARFSGSGSRTDFTLTIDPVEEDDVAVYYCLQSR | |
| | TIPRTFGGGTKLEIKTTTPAPRPPTPAPTIASQPLSLRPEACRPAAGG | |
| | AVHTRGLDFACDIYIWAPLAGTCGVLLLSLVITLYCKRGRKKLLYI | |
| | FKQPFMRPVQTTQEEDGCSCRFPEEEEGGCELRVKFSRSADAPAY | |
| | KQGQNQLYNELNLGRREEYDVLDKRRGRDPEMGGKPRRKNPQE | |
| | GLYNELQKDKMAEAYSEIGMKGERRRGKGHDGLYQGLSTATKD | |
| | TYDALHMQALPPR | |
| 167 | QIQLVQSGPELKKPGETVKISCKASGYTFTHYSMNWVKQAPGKGL | anti-BCMA |
| | KWMGRINTETGEPLYADDFKGRFAFSLETSASTAYLVINNLKNED | CAR |
| | TATFFCSNDYLYSCDYWGQGTTLTVSSGGGGSGGGGSGGGGSDIV | |
| | LTQSPPSLAMSLGKRATISCRASESVTILGSHLIYWYQQKPGQPPTL | |
| | LIQLASNVQTGVPARFSGSGSRTDFTLTIDPVEEDDVAVYYCLQSR | |
| | TIPRTFGGGTKLEIKTTTPAPRPPTPAPTIASQPLSLRPEACRPAAGG | |
| | AVHTRGLDFACDIYIWAPLAGTCGVLLLSLVITLYCKRGRKKLLYI | |
| | FKQPFMRPVQTTQEEDGCSCRFPEEEEGGCELRVKFSRSADAPAY | |
| | KQGQNQLYNELNLGRREEYDVLDKRRGRDPEMGGKPRRKNPQE | |
| | GLYNELQKDKMAEAYSEIGMKGERRRGKGHDGLYQGLSTATKD | |
| | TYDALHMQALPPR | |
| 168 | DIVLTQSPPSLAMSLGKRATISCRASESVTILGSHLIHWYQQKPGQP | anti-BCMA |
| | PTLLIQLASNVQTGVPARFSGSGSRTDFTLTIDPVEEDDVAVYYCL | CAR |
| | QSRTIPRTFGGGTKLEIKGSTSGSGKPGSGEGSTKGQIQLVQSGPEL | |
| | KKPGETVKISCKASGYTFTDYSINWVKRAPGKGLKWMGWINTET | |
| | REPAYAYDFRGRFAFSLETSASTAYLQINNLKYEDTATYFCALDYS | |
| | YAMDYWGQGTSVTVSSFVPVFLPAKPTTTPAPRPPTPAPTIASQPL | |
| | SLRPEACRPAAGGAVHTRGLDFACDIYIWAPLAGTCGVLLLSLVIT | |
| | LYCNHRNRSKRSRLLHSDYMNMTPRRPGPTRKHYQPYAPPRDFA | |
| | AYRSRVKFSRSADAPAYQQGQNQLYNELNLGRREEYDVLDKRRG | |
| | RDPEMGGKPRRKNPQEGLYNELQKDKMAEAYSEIGMKGERRRG | |
| | KGHDGLYQGLSTATKDTYDALHMQALPPR | |
| 169 | QVQLVQSGAEVKKPGASVKVSCKASGYSFPDYYINWVRQAPGQG | anti-BCMA |
| | LEWMGWIYFASGNSEYNQKFTGRVTMTRDTSINTAYMELSSLTSE | CAR |
| | DTAVYFCASLYDYDWYFDVWGQGTMVTVSSGGGGSGGGGSGG | |
| | GGSDIVMTQTPLSLSVTPGQPASISCKSSQSLVHSNGNTYLHWYLQ | |
| | KPGQSPQLLIYKVSNRFSGVPDRFSGSGSGTDFTLKISRVEAEDVGI | |
| | YYCSQSSIYPWTFGQGTKLEIKGLAVSTISSFFPPGYQIYIWAPLAG | |
| | TCGVLLLSLVITLYCKRGRKKLLYIFKQPFMRPVQTTQEEDGCSCR | |
| | FPEEEEGGCELRVKFSRSADAPAYQQGQNQLYNELNLGRREEYDV | |
| | LDKRRGRDPEMGGKPRRKNPQEGLYNELQKDKMAEAYSEIGMK | |
| | GERRRGKGHDGLYQGLSTATKDTYDALHMQALPPR | |
| 170 | QVQLVQSGAEVKKPGASVKVSCKASGYSFPDYYINWVRQAPGQG | anti-BCMA |
| | LEWMGWIYFASGNSEYNQKFTGRVTMTRDTSINTAYMELSSLTSE | CAR |
| | DTAVYFCASLYDYDWYFDVWGQGTMVTVSSGGGGSGGGGSGG | |
| | GGSDIVMTQTPLSLSVTPGQPASISCKSSQSLVHSNGNTYLHWYLQ | |
| | KPGQSPQLLIYKVSNRFSGVPDRFSGSGSGTDFTLKISRVEAEDVGI | |
| | YYCSQSSIYPWTFGQGTKLEIKTTTPAPRPPTPAPTIASQPLSLRPEA | |
| | CRPAAGGAVHTRGLDFACDIYIWAPLAGTCGVLLLSLVITLYCKR | |
| | GRKKLLYIFKQPFMRPVQTTQEEDGCSCRFPEEEEGGCELRVKFSR | |
| | SADAPAYQQGQNQLYNELNLGRREEYDVLDKRRGRDPEMGGKP | |
| | RRKNPQEGLYNELQKDKMAEAYSEIGMKGERRRGKGHDGLYQG | |
| | LSTATKDTYDALHMQALPPR | |
| 171 | QVQLVQSGAEVKKPGASVKVSCKASGYSFPDYYINWVRQAPGQG | anti-BCMA |
| | LEWMGWIYFASGNSEYNQKFTGRVTMTRDTSINTAYMELSSLTSE | CAR |
| | DTAVYFCASLYDYDWYFDVWGQGTMVTVSSGGGGSGGGGSGG | |
| | GGSDIVMTQTPLSLSVTPGQPASISCKSSQSLVHSNGNTYLHWYLQ | |
| | KPGQSPQLLIYKVSNRFSGVPDRFSGSGSGTDFTLKISRVEAEDVGI | |
| | YYCSQSSIYPWTFGQGTKLEIKEPKSPDKTHTCPPCPAPPVAGPSVF | |
| | LFPPKPKDTLMIARTPEVTCVVVDVSHEDPEVKFNWYVDGVEVH | |
| | NAKTKPREEQYNSTYRVVSVLTVLHQDWLNGKEYKCKVSNKALP | |
| | APIEKTISKAKGQPREPQVYTLPPSRDELTKNQVSLTCLVKGFYPSD | |
| | IAVEWESNGQPENNYKTTPPVLDSDGSFFLYSKLTVDKSRWQQGN | |
| | VFSCSVMHEALHNHYTQKSLSLSPGKIYIWAPLAGTCGVLLLSLVI | |
| | TLYCKRGRKKLLYIFKQPFMRPVQTTQEEDGCSCRFPEEEEGGCEL | |
| | RVKFSRSADAPAYQQGQNQLYNELNLGRREEYDVLDKRRGRDPE | |
| | MGGKPRRKNPQEGLYNELQKDKMAEAYSEIGMKGERRRGKGHD | |
| | GLYQGLSTATKDTYDALHMQALPPR | |
| 172 | QVQLVQSGAEVKKPGASVKVSCKASGYSFPDYYINWVRQAPGQG | anti-BCMA |
| | LEWMGWIYFASGNSEYNQKFTGRVTMTRDTSSSTAYMELSSLRSE | CAR |
| | DTAVYFCASLYDYDWYFDVWGQGTMVTVSSGGGGSGGGGSGG | |
| | GGSDIVMTQTPLSLSVTPGEPASISCKSSQSLVHSNGNTYLHWYLQ | |
| | KPGQSPQLLIYKVSNRFSGVPDRFSGSGSGADFTLKISRVEAEDVG | |
| | VYYCAETSHVPWTFGQGTKLEIKGLAVSTISSFFPPGYQIYIWAPLA | |
| | GTCGVLLLSLVITLYCKRGRKKLLYIFKQPFMRPVQTTQEEDGCSC | |
| | RFPEEEEGGCELRVKFSRSADAPAYQQGQNQLYNELNLGRREEYD | |
| | VLDKRRGRDPEMGGKPRRKNPQEGLYNELQKDKMAEAYSEIGM | |
| | KGERRRGKGHDGLYQGLSTATKDTYDALHMQALPPR | |
| 173 | QVQLVQSGAEVKKPGASVKVSCKASGYSFPDYYINWVRQAPGQG | anti-BCMA |
| | LEWMGWIYFASGNSEYNQKFTGRVTMTRDTSSSTAYMELSSLRSE | CAR |
| | DTAVYFCASLYDYDWYFDVWGQGTMVTVSSGGGGSGGGGSGG | |
| | GGSDIVMTQTPLSLSVTPGEPASISCKSSQSLVHSNGNTYLHWYLQ | |
| | KPGQSPQLLIYKVSNRFSGVPDRFSGSGSGADFTLKISRVEAEDVG | |
| | VYYCAETSHVPWTFGQGTKLEIKTTTPAPRPPTPAPTIASQPLSLRP | |
| | EACRPAAGGAVHTRGLDFACDIYIWAPLAGTCGVLLLSLVITLYC | |
| | KRGRKKLLYIFKQPFMRPVQTTQEEDGCSCRFPEEEEGGCELRVKF | |
| | SRSADAPAYQQGQNQLYNELNLGRREEYDVLDKRRGRDPEMGG | |
| | KPRRKNPQEGLYNELQKDKMAEAYSEIGMKGERRRGKGHDGLY | |
| | QGLSTATKDTYDALHMQALPPR | |
| 174 | QVQLVQSGAEVKKPGASVKVSCKASGYSFPDYYINWVRQAPGQG | anti-BCMA |
| | LEWMGWIYFASGNSEYNQKFTGRVTMTRDTSSSTAYMELSSLRSE | CAR |
| | DTAVYFCASLYDYDWYFDVWGQGTMVTVSSGGGGSGGGGSGG | |
| | GGSDIVMTQTPLSLSVTPGEPASISCKSSQSLVHSNGNTYLHWYLQ | |
| | KPGQSPQLLIYKVSNRFSGVPDRFSGSGSGADFTLKISRVEAEDVG | |
| | VYYCAETSHVPWTFGQGTKLEIKEPKSPDKTHTCPPCPAPPVAGPS | |
| | VFLFPPKPKDTLMIARTPEVTCVVVDVSHEDPEVKFNWYVDGVEV | |
| | HNAKTKPREEQYNSTYRVVSVLTVLHQDWLNGKEYKCKVSNKA | |
| | LPAPIEKTISKAKGQPREPQVYTLPPSRDELTKNQVSLTCLVKGFYP | |
| | SDIAVEWESNGQPENNYKTTPPVLDSDGSFFLYSKLTVDKSRWQQ | |
| | GNVFSCSVMHEALHNHYTQKSLSLSPGKIYIWAPLAGTCGVLLLS | |
| | LVITLYCKRGRKKLLYIFKQPFMRPVQTTQEEDGCSCRFPEEEEGG | |
| | CELRVKFSRSADAPAYQQGQNQLYNELNLGRREEYDVLDKRRGR | |
| | DPEMGGKPRRKNPQEGLYNELQKDKMAEAYSEIGMKGERRRGK | |
| | GHDGLYQGLSTATKDTYDALHMQALPPR | |
| 175 | IYIWAPLAGTCGVLLLSLVITLYCNHRN | CD8a TM |
| 176 | IYIWAPLAGTCGVLLLSLVIT | CD8a TM |
| 177 | RAAA | linking |
| | | peptide |
| 178 | EVQLVESGGGLVKPGGSLRLSCAASGFTFSDYYMSWIRQAPGKGL | Variable |
| | EWVSYISSSGSTIYYADSVKGRFTISRDNAKNSLYLQMNSLRAEDT | heavy (VH) |
| | AVYYCAKVDGDYTEDYWGQGTLVTVSS | Anti-BCMA |
| 179 | QSALTQPASVSGSPGQSITISCTGSSSDVGKYNLVSWYQQPPGKAP | Variable light |
| | KLIIYDVNKRPSGVSNRFSGSKSGNTATLTISGLQGDDEADYYCSS | (VL) Anti- |
| | YGGSRSYVFGTGTKVTVL | BCMA |
| 180 | EVQLVQSGGGLVQPGRSLRLSCTASGFTFGDYAMSWFRQAPGKG | Variable |
| | LEWVGFIRSKAYGGTTEYAASVKGRFTISRDDSKSIAYLQMNSLKT | heavy (VH) |
| | EDTAVYYCAAWSAPTDYWGQGTLVTVSS | Anti-BCMA |
| 181 | DIQMTQSPAFLSASVGDRVTVTCRASQGISNYLAWYQQKPGNAPR | Variable light |
| | LLIYSASTLQSGVPSRFRGTGYGTEFSLTIDSLQPEDFATYYCQQSY | (VL) Anti- |
| | TSRQTFGPGTRLDIK | BCMA |
| 182 | EVQLVESGGGLVKPGGSLRLSCAASGFTFSDYYMSWIRQAPGKGL | Variable |
| | EWVSYISSSGSTIYYADSVKGRFTISRDNAKNSLYLQMNSLRAEDT | heavy (VH) |
| | AVYYCAKVDGPPSFDIWGQGTMVTVSS | Anti-BCMA |
| 183 | SYVLTQPPSVSVAPGQTARITCGANNIGSKSVHWYQQKPGQAPML | Variable light |
| | VVYDDDDRPSGIPERFSGSNSGNTATLTISGVEAGDEADYFCHLW | (VL) Anti- |
| | DRSRDHYVFGTGTKLTVL | BCMA |
| 184 | EVQLVQSGAEVKKPGSSVKVSCKASGGTFSSYAISWVRQAPGQGL | Variable |
| | EWMGRIIPILGIANYAQKFQGRVTMTEDTSTDTAYMELSSLRSEDT | heavy (VH) |
| | AVYYCARSGYSKSIVSYMDYWGQGTLVTVSS | Anti-BCMA |
| 185 | LPVLTQPPSTSGTPGQRVTVSCSGSSSNIGSNVVFWYQQLPGTAPK | Variable light |
| | LVIYRNNQRPSGVPDRFSVSKSGTSASLAISGLRSEDEADYYCAAW | (VL) Anti- |
| | DDSLSGYVFGTGTKVTVLG | BCMA |
| 186 | MPLLLLLPLLWAGALA | CD33 Signal |
| | | peptide |
| 187 | MALPVTALLLPLALLLHA | CD8 alpha |
| | | signal |
| | | peptide |
| 188 | atgcttctcctggtgacaagccttctgctctgtgagttaccacacccagcattcctcctgatccca | GMCSFR |
| | | alpha chain |
| | | signal |
| | | sequence |
| 189 | MLLLVTSLLLCELPHPAFLLIP | GMCSFR |
| | | alpha chain |
| | | signal |
| | | sequence |
| 190 | Glu Val Val Val Lys Tyr Gly Pro Pro Cys Pro Pro Cys Pro | Exemplary |
| | | IgG Hinge |
| 191 | X1PPX2P | Exemplary |
| | X1 is glycine, cysteine or arginine | IgG Hinge |
| | X2 is cysteine or threonine | |
| 192 | Glu Pro Lys Ser Cys Asp Lys Thr His Thr Cys Pro Pro Cys Pro | Exemplary |
| | | IgG Hinge |
| 193 | Glu Arg Lys Cys Cys Val Glu Cys Pro Pro Cys Pro | Exemplary |
| | | IgG Hinge |
| 194 | ELKTPLGDTHTCPRCPEPKSCDTPPPCPRCPEPKSCDTPPPCPRCPEP | Exemplary |
| | KSCDTPPPCPRCP | IgG Hinge |
| 195 | Glu Ser Lys Tyr Gly Pro Pro Cys Pro Ser Cys Pro | Exemplary |
| | | IgG Hinge |
| 196 | Glu Ser Lys Tyr Gly Pro Pro Cys Pro Pro Cys Pro | Exemplary |
| | | IgG Hinge |
| 197 | Tyr Gly Pro Pro Cys Pro Pro Cys Pro | Exemplary |
| | | IgG Hinge |
| 198 | Lys Tyr Gly Pro Pro Cys Pro Pro Cys Pro | Exemplary |
| | | IgG Hinge |
| 199 | MLLLVTSLLLCELPHPAFLLIPRKVCNGIGIGEFKDSLSINATNIKHF | tEGFR |
| | KNCTSISGDLHILPVAFRGDSFTHTPPLDPQELDILKTVKEITGFLLI | artificial |
| | QAWPENRTDLHAFENLEIIRGRTKQHGQFSLAVVSLNITSLGLRSL | |
| | KEISDGDVIISGNKNLCYANTINWKKLFGTSGQKTKIISNRGENSCK | |
| | ATGQVCHALCSPEGCWGPEPRDCVSCRNVSRGRECVDKCNLLEG | |
| | EPREFVENSECIQCHPECLPQAMNITCTGRGPDNCIQCAHYIDGPH | |
| | CVKTCPAGVMGENNTLVWKYADAGHVCHLCHPNCTYGCTGPGL | |
| | EGCPTNGPKIPSIATGMVGALLLLLVVALGIGLFM | |
| 200 | EGRGSLLTCGDVEENPGP | T2A |
| | | artificial |
| 201 | GSGATNFSLLKQAGDVEENPGP | P2A |
| 202 | ATNFSLLKQAGDVEENPGP | P2A |
| 203 | QCTNYALLKLAGDVESNPGP | E2A |
| 204 | VKQTLNFDLLKLAGDVESNPGP | F2A |
| 205 | MLQMAGQCSQNEYFDSLLHACIPCQLRCSSNTPPLTCQRYCNASV | BCMA-Fc |
| | TNSVKGTNAGGGGSPKSSDKTHTCPPCPAPEAEGAPSVFLFPPKPK | fusion |
| | DTLMISRTPEVTCVVVDVSHEDPEVKFNWYVDGVEVHNAKTKPR | polypeptide |
| | EEQYNSTYRVVSVLTVLHQDWLNGKEYKCKVSNKALPSSIEKTIS | |
| | KAKGQPREPQVYTLPPSRDELTKNQVSLTCLVKGFYPSDIAVEWE | |
| | SNGQPENNYKTTPPVLDSDGSFFLYSKLTVDKSRWQQGNVFSCSV | |
| | MHEALHNHYTQKSLSLSPGK | |
| 206 | DYYVY | CDR-H1 |
| 207 | WINPNSGGTNYAQKFQG | CDR-H2 |
| 208 | SQRDGYMDY | CDR-H3 |
| 209 | GYTFIDY | CDR-H1 |
| 210 | NPNSGG | CDR-H2 |
| 211 | GYTFIDYYVY | CDR-H1 |
| 212 | WINPNSGGTN | CDR-H2 |
| 213 | GYTFIDYY | CDR-H1 |
| 214 | INPNSGGT | CDR-H2 |
| 215 | ARSQRDGYMDY | CDR-H3 |
| 216 | TGTSSDVG | CDR-L1 |
| 217 | EDSKRPS | CDR-L2 |
| 218 | SSNTRSSTLV | CDR-L3 |
| 219 | ISCTGTSSD | CDR-L1 |
| 220 | EDS | CDR-L2 |
| 221 | QSALTQPASVSASPGQSIAISCTGTSSDVGWYQQHPGKAPKLMIYE | anti-BCMA |
| | DSKRPSGVSNRFSGSKSGNTASLTISGLQAEDEADYYCSSNTRSST | scFv |
| | LVFGGGTKLTVLGSRGGGGSGGGGSGGGGSLEMAEVQLVQSGAE | |
| | MKKPGASLKLSCKASGYTFIDYYVYWMRQAPGQGLESMGWINPN | |
| | SGGTNYAQKFQGRVTMTRDTSISTAYMELSRLRSDDTAMYYCAR | |
| | SQRDGYMDYWGQGTLVTVSS | |
| 222 | GYTFTDY | CDR-H1 |
| 223 | NTETRE | CDR-H2 |
| 224 | DYSYAMDY | CDR-H3 |
| 225 | RASESVTILGSHLIH | CDR-L1 |
| 226 | LASNVQT | CDR-L2 |
| 227 | LQSRTIPRT | CDR-L3 |
| 228 | GYTFTDYSIN | CDR-H1 |
| 229 | WINTETREPA | CDR-H2 |
| 230 | DYSYAMDY | CDR-H3 |
| 231 | RASESVTILGSHLIH | CDR-L1 |
| 232 | LASNVQT | CDR-L2 |
| 233 | LQSRTIPRT | CDR-L3 |
| 234 | GYTFTDYS | CDR-H1 |
| 235 | INTETREP | CDR-H2 |
| 236 | ALDYSYAMDY | CDR-H3 |
| 237 | ESVTILGSHL | CDR-L1 |
| 238 | LA | CDR-L2 |
| 239 | LQSRTIPRT | CDR-L3 |
| 240 | atggcactcc ccgtcaccgc ccttctcttg cccctcgccc tgctgctgca tgctgccagg | BCMA CAR |
| | cccgacattg tgctcactca gtcacctccc agcctggcca tgagcctggg aaaaagggcc | (nt) |
| | accatctcct gtagagccag tgagtccgtc acaatcttgg ggagccatct tattcactgg | |
| | tatcagcaga agcccgggca gcctccaacc cttcttattc agctcgcgtc aaacgtccag | |
| | acgggtgtac ctgccagatt ttctggtagc gggtcccgca ctgattttac actgaccata | |
| | gatccagtgg aagaagacga tctggccgtg tattattgtc tgcagagcag aacgattcct | |
| | cgcacatttg gtgggggtac taagctggag attaagggaa gcacgtccgg ctcagggaag | |
| | ccgggctccg gcgagggaag cacgaagggg caaattcagc tggtccagag cggacctgag | |
| | ctgaaaaaac ccggcgagac tgttaagatc agttgtaaag catctggcta taccttcacc | |
| | gactacagca taaattgggt gaaacgggcc cctggaaagg gcctcaaatg gatgggttgg | |
| | atcaataccg aaactaggga gcctgcttat gcatatgact tccgcgggag attcgccttt | |
| | tcactcgaga catctgcctc tactgcttac ctccaaataa acaacctcaa gtatgaagat | |
| | acagccactt acttttgcgc cctcgactat agttacgcca tggactactg gggacaggga | |
| | acctccgtta ccgtcagttc cgcggccgca accacaacac ctgctccaag gccccccaca | |
| | cccgctccaa ctatagccag ccaaccattg agcctcagac ctgaagcttg caggcccgca | |
| | gcaggaggcg ccgtccatac gcgaggcctg gacttcgcgt gtgatattta tatttgggcc | |
| | cctttggccg gaacatgtgg ggtgttgctt ctctcccttg tgatcactct gtattgtaag | |
| | cgcgggagaa agaagctcct gtacatcttc aagcagcctt ttatgcgacc tgtgcaaacc | |
| | actcaggaag aagatgggtg ttcatgccgc ttccccgagg aggaagaagg agggtgtgaa | |
| | ctgagggtga aattttctag aagcgccgat gctcccgcat atcagcaggg tcagaatcag | |
| | ctctacaatg aattgaatct cggcaggcga gaagagtacg atgttctgga caagagacgg | |
| | ggcagggatc ccgagatggg gggaaagccc cggagaaaaa atcctcagga ggggttgtac | |
| | aatgagctgc agaaggacaa gatggctgaa gcctatagcg agatcggaat gaaaggcgaa | |
| | agacgcagag gcaaggggca tgacggtctg taccagggtc tctctacagc caccaaggac | |
| | acttatgatg cgttgcatat gcaagccttg ccaccccgct aatga | |
| 241 | EVQLLESGGGLVQPGGSLRLSCAASGFTFSSYAMSWVRQAPGKGL | Anti-BCMA |
| | EWVSAISGSGGSTYYADSVKGRFTISRDNSKNTLYLQMNSLRAED | scFv |
| | TAVYYCARAEMGAVFDIWGQGTMVTVSSGSTSGSGKPGSGEGST | |
| | KGEIVLTQSPATLSLSPGERATLSCRASQSVSRYLAWYQQKPGQAP | |
| | RLLIYDASNRATGIPARFSGSGSGTDFTLTISSLEPEDFAVYYCQQRI | |
| | SWPFTFGGGTKVEIK | |
| 242 | EIVLTQSPATLSLSPGERATLSCRASQSVSRYLAWYQQKPGQAPRL | Anti-BCMA |
| | LIYDASNRATGIPARFSGSGSGTDFTLTISSLEPEDFAVYYCQQRIS | scFv |
| | WPFTFGGGTKVEIKRGSTSGSGKPGSGEGSTKGEVQLLESGGGLV | |
| | QPGGSLRLSCAASGFTFSSYAMSWVRQAPGKGLEWVSAISGSGGS | |
| | TYYADSVKGRFTISRDNSKNTLYLQMNSLRAEDTAVYYCARAEM | |
| | GAVFDIWGQGTMVTVSS | |
| 243 | QVQLVESGGGVVQPGRSLRLSCAASGFTFSSYGMHWVRQAPGKG | Anti-BCMA |
| | LEWVAVISYDGSNKYYADSVKGRFTISRDNSKNTLYLQMNSLRAE | scFv |
| | DTAVYYCARDGTYLGGLWYFDLWGRGTLVTVSSGSTSGSGKPGS | |
| | GEGSTKGDIVMTQSPLSLPVTPGEPASISCRSSQSLLHSNGYNYLD | |
| | WYLQKPGQSPQLLIYLGSNRASGVPDRFSGSGSGTDFTLKISRVEA | |
| | EDVGVYYCMQGLGLPLTFGGGTKVEIK | |
| 244 | DIVMTQSPLSLPVTPGEPASISCRSSQSLLHSNGYNYLDWYLQKPG | Anti-BCMA |
| | QSPQLLIYLGSNRASGVPDRFSGSGSGTDFTLKISRVEAEDVGVYY | scFv |
| | CMQGLGLPLTFGGGTKVEIKRGSTSGSGKPGSGEGSTKGQVQLVE | |
| | SGGGVVQPGRSLRLSCAASGFTFSSYGMHWVRQAPGKGLEWVAV | |
| | ISYDGSNKYYADSVKGRFTISRDNSKNTLYLQMNSLRAEDTAVYY | |
| | CARDGTYLGGLWYFDLWGRGTLVTVSS | |
| 245 | QVQLVQSGAEVKKPGASVKVSCKASGYTFTSYYMHWVRQAPGQ | Anti-BCMA |
| | GLEWMGIINPGGGSTSYAQKFQGRVTMTRDTSTSTVYMELSSLRS | scFv |
| | EDTAVYYCARESWPMDVWGQGTTVTVSSGSTSGSGKPGSGEGST | |
| | KGEIVMTQSPATLSVSPGERATLSCRASQSVSSNLAWYQQKPGQA | |
| | PRLLIYGASTRATGIPARFSGSGSGTEFTLTISSLQSEDFAVYYCQQ | |
| | YAAYPTFGGGTKVEIK | |
| 246 | EIVMTQSPATLSVSPGERATLSCRASQSVSSNLAWYQQKPGQAPR | Anti-BCMA |
| | LLIYGASTRATGIPARFSGSGSGTEFTLTISSLQSEDFAVYYCQQYA | scFv |
| | AYPTFGGGTKVEIKRGSTSGSGKPGSGEGSTKGQVQLVQSGAEVK | |
| | KPGASVKVSCKASGYTFTSYYMHWVRQAPGQGLEWMGIINPGGG | |
| | STSYAQKFQGRVTMTRDTSTSTVYMELSSLRSEDTAVYYCARESW | |
| | PMDVWGQGTTVTVSS | |
| 247 | QLQLQESGPGLVKPSETLSLTCTVSGGSISSSSYYWGWIRQPPGKG | Anti-BCMA |
| | LEWIGSISYSGSTYYNPSLKSRVTISVDTSKNQFSLKLSSVTAADTA | scFv |
| | VYYCARGRGYATSLAFDIWGQGTMVTVSSGSTSGSGKPGSGEGST | |
| | KGEIVLTQSPATLSLSPGERATLSCRASQSVSSYLAWYQQKPGQAP | |
| | RLLIYDASNRATGIPARFSGSGSGTDFTLTISSLEPEDFAVYYCQQR | |
| | HVWPPTFGGGTKVEIK | |
| 248 | EIVLTQSPATLSLSPGERATLSCRASQSVSSYLAWYQQKPGQAPRL | Anti-BCMA |
| | LIYDASNRATGIPARFSGSGSGTDFTLTISSLEPEDFAVYYCQQRHV | scFv |
| | WPPTFGGGTKVEIKRGSTSGSGKPGSGEGSTKGQLQLQESGPGLV | |
| | KPSETLSLTCTVSGGSISSSSYYWGWIRQPPGKGLEWIGSISYSGST | |
| | YYNPSLKSRVTISVDTSKNQFSLKLSSVTAADTAVYYCARGRGYA | |
| | TSLAFDIWGQGTMVTVSS | |
| 249 | EVQLVESGGGLVQPGGSLRLSCAASGFTFSSYSMNWVRQAPGKG | Anti-BCMA |
| | LEWVSTISSSSSTIYYADSVKGRFTISRDNAKNSLYLQMNSLRAED | scFv |
| | TAVYYCARGSQEHLIFDYWGQGTLVTVSSGSTSGSGKPGSGEGST | |
| | KGEIVLTQSPATLSLSPGERATLSCRASQSVSRYLAWYQQKPGQAP | |
| | RLLIYDASNRATGIPARFSGSGSGTDFTLTISSLEPEDFAVYYCQQR | |
| | FYYPWTFGGGTKVEIK | |
| 250 | EIVLTQSPATLSLSPGERATLSCRASQSVSRYLAWYQQKPGQAPRL | Anti-BCMA |
| | LIYDASNRATGIPARFSGSGSGTDFTLTISSLEPEDFAVYYCQQRFY | scFv |
| | YPWTFGGGTKVEIKRGSTSGSGKPGSGEGSTKGEVQLVESGGGLV | |
| | QPGGSLRLSCAASGFTFSSYSMNWVRQAPGKGLEWVSTISSSSSTI | |
| | YYADSVKGRFTISRDNAKNSLYLQMNSLRAEDTAVYYCARGSQE | |
| | HLIFDYWGQGTLVTVSS | |
| 251 | QVQLVESGGGVVQPGRSLRLSCAASGFTFSSYGMHWVRQAPGKG | Anti-BCMA |
| | LEWVAVISYDGSNKYYADSVKGRFTISRDNSKNTLYLQMNSLRAE | scFv |
| | DTAVYYCARTDFWSGSPPGLDYWGQGTLVTVSSGSTSGSGKPGS | |
| | GEGSTKGDIQLTQSPSSVSASVGDRVTITCRASQGISSWLAWYQQK | |
| | PGKAPKLLIYGASSLQSGVPSRFSGSGSGTDFTLTISSLQPEDFATY | |
| | YCQQIYTFPFTFGGGTKVEIK | |
| 252 | DIQLTQSPSSVSASVGDRVTITCRASQGISSWLAWYQQKPGKAPKL | Anti-BCMA |
| | LIYGASSLQSGVPSRFSGSGSGTDFTLTISSLQPEDFATYYCQQIYTF | scFv |
| | PFTFGGGTKVEIKRGSTSGSGKPGSGEGSTKGQVQLVESGGGVVQ | |
| | PGRSLRLSCAASGFTFSSYGMHWVRQAPGKGLEWVAVISYDGSN | |
| | KYYADSVKGRFTISRDNSKNTLYLQMNSLRAEDTAVYYCARTDF | |
| | WSGSPPGLDYWGQGTLVTVSS | |
| 253 | QVQLVQSGAEVKKPGSSVKVSCKASGGTFSSYAISWVRQAPGQGL | Anti-BCMA |
| | EWMGGIIPIFGTANYAQKFQGRVTITADESTSTAYMELSSLRSEDT | scFv |
| | AVYYCARTPEYSSSIWHYYYGMDVWGQGTTVTVSSGSTSGSGKP | |
| | GSGEGSTKGDIVMTQSPDSLAVSLGERATINCKSSQSVLYSSNNKN | |
| | YLAWYQQKPGQPPKLLIYWASTRESGVPDRFSGSGSGTDFTLTISS | |
| | LQAEDVAVYYCQQFAHTPFTFGGGTKVEIK | |
| 254 | DIVMTQSPDSLAVSLGERATINCKSSQSVLYSSNNKNYLAWYQQK | Anti-BCMA |
| | PGQPPKLLIYWASTRESGVPDRFSGSGSGTDFTLTISSLQAEDVAV | scFv |
| | YYCQQFAHTPFTFGGGTKVEIKRGSTSGSGKPGSGEGSTKGQVQL | |
| | VQSGAEVKKPGSSVKVSCKASGGTFSSYAISWVRQAPGQGLEWM | |
| | GGIIPIFGTANYAQKFQGRVTITADESTSTAYMELSSLRSEDTAVYY | |
| | CARTPEYSSSIWHYYYGMDVWGQGTTVTVSS | |
| 255 | QVQLVESGGGVVQPGRSLRLSCAASGFTFSSYGMHWVRQAPGKG | Anti-BCMA |
| | LEWVAVISYDGSNKYYADSVKGRFTISRDNSKNTLYLQMNSLRAE | scFv |
| | DTAVYYCVKGPLQEPPYDYGMDVWGQGTTVTVSSGSTSGSGKPG | |
| | SGEGSTKGEIVMTQSPATLSVSPGERATLSCRASQSVSSNLAWYQQ | |
| | KPGQAPRLLIYSASTRATGIPARFSGSGSGTEFTLTISSLQSEDFAVY | |
| | YCQQHHVWPLTFGGGTKVEIK | |
| 256 | EIVMTQSPATLSVSPGERATLSCRASQSVSSNLAWYQQKPGQAPR | Anti-BCMA |
| | LLIYSASTRATGIPARFSGSGSGTEFTLTISSLQSEDFAVYYCQQHH | scFv |
| | VWPLTFGGGTKVEIKRGSTSGSGKPGSGEGSTKGQVQLVESGGGV | |
| | VQPGRSLRLSCAASGFTFSSYGMHWVRQAPGKGLEWVAVISYDG | |
| | SNKYYADSVKGRFTISRDNSKNTLYLQMNSLRAEDTAVYYCVKG | |
| | PLQEPPYDYGMDVWGQGTTVTVSS | |
| 257 | DIVLTQSPASLAVSLGERATINCRASESVSVIGAHLIHWYQQKPGQ | Anti-BCMA |
| | PPKLLIYLASNLETGVPARFSGSGSGTDFTLTISSLQAEDAAIYYCL | scFv |
| | QSRIFPRTFGQGTKLEIKGSTSGSGKPGSGEGSTKGQVQLVQSGSEL | |
| | KKPGASVKVSCKASGYTFTDYSINWVRQAPGQGLEWMGWINTET | |
| | REPAYAYDFRGRFVFSLDTSVSTAYLQISSLKAEDTAVYYCARDY | |
| | SYAMDYWGQGTLVTVSS | |
| 258 | DIVLTQSPASLAVSLGERATINCRASESVSVIGAHLIHWYQQKPGQ | Anti-BCMA |
| | PPKLLIYLASNLETGVPARFSGSGSGTDFTLTISSLQAEDAAIYYCL | scFv |
| | QSRIFPRTFGQGTKLEIKGSTSGSGKPGSGEGSTKGQVQLVQSGSEL | |
| | KKPGASVKVSCKASGYTFTDYSINWVRQAPGQGLEWMGWINTET | |
| | REPAYAYDFRGRFVFSLDTSVSTAYLQISSLKAEDTAVYYCARDY | |
| | SYAMDYWGQGTLVTVSS | |
| 259 | DIVLTQSPASLAVSLGERATINCRASESVSVIGAHLIHWYQQKPGQ | Anti-BCMA |
| | PPKLLIYLASNLETGVPARFSGSGSGTDFTLTISSLQAEDAAIYYCL | scFv |
| | QSRIFPRTFGQGTKLEIKGSTSGSGKPGSGEGSTKGQVQLVQSGSEL | |
| | KKPGASVKVSCKASGYTFTDYSINWVRQAPGQGLEWMGWINTET | |
| | REPAYAYDFRGRFVFSLDTSVSTAYLQISSLKAEDTAVYYCARDY | |
| | SYAMDYWGQGTLVTVSS | |
| 260 | EVQLVESGGGLVQPGGSLRLSCAVSGFALSNHGMSWVRRAPGKG | Anti-BCMA |
| | LEWVSGIVYSGSTYYAASVKGRFTISRDNSRNTLYLQMNSLRPED | scFv |
| | TAIYYCSAHGGESDVWGQGTTVTVSSASGGGGSGGRASGGGGSDI | |
| | QLTQSPSSLSASVGDRVTITCRASQSISSYLNWYQQKPGKAPKLLIY | |
| | AASSLQSGVPSRFSGSGSGTDFTLTISSLQPEDFATYYCQQSYSTPY | |
| | TFGQGTKVEIK | |
| 261 | QVQLVESGGGLVQPGRSLRLSCAASGFTFSNYAMSWVRQAPGKG | Anti-BCMA |
| | LGWVSGISRSGENTYYADSVKGRFTISRDNSKNTLYLQMNSLRDE | scFv |
| | DTAVYYCARSPAHYYGGMDVWGQGTTVTVSSASGGGGSGGRAS | |
| | GGGGSDIVLTQSPGTLSLSPGERATLSCRASQSISSSFLAWYQQKPG | |
| | QAPRLLIYGASRRATGIPDRFSGSGSGTDFTLTISRLEPEDSAVYYC | |
| | QQYHSSPSWTFGQGTKLEIK | |
| 262 | QVQLVESGGGLVQPGGSLRLSCAVSGFALSNHGMSWVRRAPGKG | Anti-BCMA |
| | LEWVSGIVYSGSTYYAASVKGRFTISRDNSRNTLYLQMNSLRPED | scFv |
| | TAIYYCSAHGGESDVWGQGTTVTVSSASGGGGSGGRASGGGGSDI | |
| | RLTQSPSPLSASVGDRVTITCQASEDINKFLNWYHQTPGKAPKLLI | |
| | YDASTLQTGVPSRFSGSGSGTDFTLTINSLQPEDIGTYYCQQYESLP | |
| | LTFGGGTKVEIK | |
| 263 | EVQLVESGGGLVQPGGSLRLSCAVSGFALSNHGMSWVRRAPGKG | Anti-BCMA |
| | LEWVSGIVYSGSTYYAASVKGRFTISRDNSRNTLYLQMNSLRPED | scFv |
| | TAIYYCSAHGGESDVWGQGTTVTVSSASGGGGSGGRASGGGGSEI | |
| | VLTQSPGTLSLSPGERATLSCRASQSIGSSSLAWYQQKPGQAPRLL | |
| | MYGASSRASGIPDRFSGSGSGTDFTLTISRLEPEDFAVYYCQQYAG | |
| | SPPFTFGQGTKVEIK | |
| 264 | QIQLVQSGPELKKPGETVKISCKASGYTFTDYSINWVKRAPGKGLK | Anti-BCMA |
| | WMGWINTETREPAYAYDFRGRFAFSLETSASTAYLQINNLKYEDT | scFv |
| | ATYFCALDYSYAMDYWGQGTSVTVSSGGGGSGGGGSGGGGSDIV | |
| | LTQSPASLAMSLGKRATISCRASESVSVIGAHLIHWYQQKPGQPPK | |
| | LLIYLASNLETGVPARFSGSGSGTDFTLTIDPVEEDDVAIYSCLQSRI | |
| | FPRTFGGGTKLEIK | |
| 265 | QIQLVQSGPELKKPGETVKISCKASGYTFRHYSMNWVKQAPGKGL | Anti-BCMA |
| | KWMGRINTESGVPIYADDFKGRFAFSVETSASTAYLVINNLKDED | scFv |
| | TASYFCSNDYLYSLDFWGQGTALTVSSGGGGSGGGGSGGGGSDIV | |
| | LTQSPPSLAMSLGKRATISCRASESVTILGSHLIYWYQQKPGQPPTL | |
| | LIQLASNVQTGVPARFSGSGSRTDFTLTIDPVEEDDVAVYYCLQSR | |
| | TIPRTFGGGTKLEIK | |
| 266 | QIQLVQSGPELKKPGETVKISCKASGYTFTHYSMNWVKQAPGKGL | Anti-BCMA |
| | KWMGRINTETGEPLYADDFKGRFAFSLETSASTAYLVINNLKNED | scFv |
| | TATFFCSNDYLYSCDYWGQGTTLTVSSGGGGSGGGGSGGGGSDIV | |
| | LTQSPPSLAMSLGKRATISCRASESVTILGSHLIYWYQQKPGQPPTL | |
| | LIQLASNVQTGVPARFSGSGSRTDFTLTIDPVEEDDVAVYYCLQSR | |
| | TIPRTFGGGTKLEIK | |
| 267 | QVQLVQSGAEVKKPGASVKVSCKASGYSFPDYYINWVRQAPGQG | Anti-BCMA |
| | LEWMGWIYFASGNSEYNQKFTGRVTMTRDTSINTAYMELSSLTSE | scFv |
| | DTAVYFCASLYDYDWYFDVWGQGTMVTVSSGGGGSGGGGSGG | |
| | GGSDIVMTQTPLSLSVTPGQPASISCKSSQSLVHSNGNTYLHWYLQ | |
| | KPGQSPQLLIYKVSNRFSGVPDRFSGSGSGTDFTLKISRVEAEDVGI | |
| | YYCSQSSIYPWTFGQGTKLEIK | |
| 268 | QVQLVQSGAEVKKPGASVKVSCKASGYSFPDYYINWVRQAPGQG | Anti-BCMA |
| | LEWMGWIYFASGNSEYNQKFTGRVTMTRDTSINTAYMELSSLTSE | scFv |
| | DTAVYFCASLYDYDWYFDVWGQGTMVTVSSGGGGSGGGGSGG | |
| | GGSDIVMTQTPLSLSVTPGQPASISCKSSQSLVHSNGNTYLHWYLQ | |
| | KPGQSPQLLIYKVSNRFSGVPDRFSGSGSGTDFTLKISRVEAEDVGI | |
| | YYCSQSSIYPWTFGQGTKLEIK | |
| 269 | QVQLVQSGAEVKKPGASVKVSCKASGYSFPDYYINWVRQAPGQG | Anti-BCMA |
| | LEWMGWIYFASGNSEYNQKFTGRVTMTRDTSINTAYMELSSLTSE | scFv |
| | DTAVYFCASLYDYDWYFDVWGQGTMVTVSSGGGGSGGGGSGG | |
| | GGSDIVMTQTPLSLSVTPGQPASISCKSSQSLVHSNGNTYLHWYLQ | |
| | KPGQSPQLLIYKVSNRFSGVPDRFSGSGSGTDFTLKISRVEAEDVGI | |
| | YYCSQSSIYPWTFGQGTKLEIK | |
| 270 | QVQLVQSGAEVKKPGASVKVSCKASGYSFPDYYINWVRQAPGQG | Anti-BCMA |
| | LEWMGWIYFASGNSEYNQKFTGRVTMTRDTSSSTAYMELSSLRSE | scFv |
| | DTAVYFCASLYDYDWYFDVWGQGTMVTVSSGGGGSGGGGSGG | |
| | GGSDIVMTQTPLSLSVTPGEPASISCKSSQSLVHSNGNTYLHWYLQ | |
| | KPGQSPQLLIYKVSNRFSGVPDRFSGSGSGADFTLKISRVEAEDVG | |
| | VYYCAETSHVPWTFGQGTKLEIK | |
| 271 | QVQLVQSGAEVKKPGASVKVSCKASGYSFPDYYINWVRQAPGQG | Anti-BCMA |
| | LEWMGWIYFASGNSEYNQKFTGRVTMTRDTSSSTAYMELSSLRSE | scFv |
| | DTAVYFCASLYDYDWYFDVWGQGTMVTVSSGGGGSGGGGSGG | |
| | GGSDIVMTQTPLSLSVTPGEPASISCKSSQSLVHSNGNTYLHWYLQ | |
| | KPGQSPQLLIYKVSNRFSGVPDRFSGSGSGADFTLKISRVEAEDVG | |
| | VYYCAETSHVPWTFGQGTKLEIK | |
| 272 | QVQLVQSGAEVKKPGASVKVSCKASGYSFPDYYINWVRQAPGQG | Anti-BCMA |
| | LEWMGWIYFASGNSEYNQKFTGRVTMTRDTSSSTAYMELSSLRSE | scFv |
| | DTAVYFCASLYDYDWYFDVWGQGTMVTVSSGGGGSGGGGSGG | |
| | GGSDIVMTQTPLSLSVTPGEPASISCKSSQSLVHSNGNTYLHWYLQ | |
| | KPGQSPQLLIYKVSNRFSGVPDRFSGSGSGADFTLKISRVEAEDVG | |
| | VYYCAETSHVPWTFGQGTKLEIK | |
| 273 | IYIWAPLAGTCGVLLLSLVITLYC | CD8alpha |
| | | TM |
| 274 | AAA | Linker |

What is claimed:

1. A method of predicting whether a subject will exhibit a clinical response to a T cell therapy, comprising:
(a) obtaining, for a subject having a disease or condition, a parameter of a marker or parameters of a combination of markers, wherein:
(i) the parameter or parameters are obtained prior to the subject being administered a T cell therapy comprising T cells comprising a recombinant receptor that binds to an antigen associated with the disease or condition; and
(ii) the marker or the combination of markers is selected from the (1) level in a blood sample of d-dimer, (2) level in a blood sample of fibrinogen, (3) level in a blood sample of lymphocytes, (4) level in a blood sample of monocytes, (5) ratio in a blood sample of monocytes to leukocytes, (6) level in a blood sample of red blood cells, (7) level in a blood sample of white blood cells, (8) age, (9) body mass index, (10) level in a blood sample of albumin, (11) level in a blood sample of alkaline phosphatase, (12) level in a blood sample of aspartate aminotransferase, (13) level in a blood sample of alanine aminotransferase, (14) level in a blood sample of direct bilirubin, (15) level in a blood sample of bilirubin, (16) level in a blood sample of creatinine, (17) creatinine clearance, (18) time since diagnosis, (19) number of prior therapies received, (20) time since prior autologous stem cell transplant, (21) time since prior corticosteroid therapy, (22) time since prior alkylating agent therapy, (23) time since prior topoisomerase inhibitor therapy, (24) time since prior proteasome inhibitor therapy, (25) percent in a blood sample of bone marrow plasma cells, (26) level in a blood sample of beta-2 microglobulin, (27) level in a blood sample of Immunoglobulin G, (28) level in a blood sample of lactate dehydrogenase, (29) ratio in a blood sample of kappa to lambda free light chain levels, (30) level in a blood sample of free light chain, (31) level in a blood sample of M-protein, (32) level in a blood sample of platelets, (33) level in a blood sample of sodium, and (34) level in a blood sample of soluble BCMA of the subject; and
(b) predicting if the subject is likely to exhibit a clinical response to administration of the T cell therapy for treatment of the disease or condition, wherein the subject is predicted as likely to exhibit the clinical response if:
(i) the parameter or one or more of the parameters for markers (3), (6)-(13), (16), (17), (20), (22)-(24), (28), (29), (32), and (33) are higher than an associated threshold level; or
(ii) the parameter or one or more of the parameters for markers (1), (2), (4), (5), (14), (15), (18), (19), (21), (25)-(27), (30), (31), and (34) are lower than an associated threshold level.

2. The method of claim 1, wherein the subject is predicted as likely to exhibit the clinical response if two or more, three or more, or four or more of any of the criteria of step (b)(i)-(b)(ii) are satisfied.

3. A method of predicting whether a subject will not exhibit a clinical response to a T cell therapy, comprising:
(a) obtaining, for a subject having a disease or condition, a parameter of a marker or parameters of a combination of markers, wherein:
(i) the parameter or parameters are obtained prior to the subject being administered a T cell therapy comprising T cells comprising a recombinant receptor that binds to an antigen associated with the disease or condition; and
(ii) the marker or the combination of markers is selected from the (1) level in a blood sample of d-dimer, (2) level in a blood sample of fibrinogen, (3) level in a blood sample of lymphocytes, (4) level in a blood sample of monocytes, (5) ratio in a blood sample of monocytes to leukocytes, (6) level in a blood sample of red blood cells, (7) level in a blood sample of white blood cells, (8) age, (9) body mass index, (10) level in a blood sample of albumin, (11) level in a blood sample of alkaline phosphatase, (12) level in a blood sample of aspartate aminotransferase, (13) level in a blood sample of alanine aminotransferase, (14) level in a blood sample of direct bilirubin, (15) level in a blood sample of bilirubin, (16) level in a blood sample of creatinine, (17) creatinine clearance, (18) time since diagnosis, (19) number of prior therapies received, (20) time since prior autologous stem cell transplant, (21) time since prior corticosteroid therapy, (22) time since prior alkylating agent therapy, (23) time since prior topoisomerase inhibitor therapy, (24) time since prior proteasome inhibitor therapy, (25) percent in a blood sample of bone marrow plasma cells, (26) level in a blood sample of beta-2 microglobulin, (27) level in a blood sample of Immunoglobulin G, (28) level in a blood sample of lactate dehydrogenase, (29) ratio in a blood sample of kappa to lambda free light chain levels, (30) level in a blood sample of free light chain, (31) level in a blood sample of M-protein, (32) level in a blood sample of platelets, (33) level in a blood sample of sodium, and (34) level in a blood sample of soluble BCMA of the subject; and
(b) predicting if the subject is likely to not exhibit a clinical response to administration of the T cell therapy for treatment of the disease or condition, wherein the subject is predicted as likely to not exhibit the clinical response if:
(i) the parameter or one or more of the parameters for markers (3), (6)-(13), (16), (17), (20), (22)-(24), (28), (29), (32), and (33) are lower than an associated threshold level; or
(ii) the parameter or one or more of the parameters for markers (1), (2), (4), (5), (14), (15), (18), (19), (21), (25)-(27), (30), (31), and (34) are higher than an associated threshold level.

4. The method of claim 3, wherein the subject is predicted as likely to not exhibit the clinical response if two or more, three or more, or four or more of any of the criteria of step (b)(i)-(b)(ii) are satisfied.

5. The method of any one of claims 1-4, wherein the marker is or the combination of markers comprises one or more subject immune profile markers that are selected from markers (1)-(7).

6. The method of any one of claims 1-5, wherein the marker is or the combination of markers comprises one or more subject fitness markers that are selected from markers (8)-(17).

7. The method of any one of claims 1-6, wherein the marker is or the combination of markers comprises one or more subject prior therapy markers that are selected from markers (18)-(24).

8. The method of any one of claims 1-7, wherein the marker is or the combination of markers comprises one or more subject tumor burden markers that are selected from markers (25)-(34).

9. The method of any one of claims 1-8, wherein:
the threshold level associated with marker (1) is between or between about 0.5 mg/L and 11 mg/L or between or between about 0.5 mg/L and 1.3 mg/L;
the threshold level associated with marker (2) is between or between about 2.2 g/L and 7.7 g/L or between or between about 4.2 g/L and 5.4 g/L;
the threshold level associated with marker (3) is between or between about 0.3×10⁹ cells/L and 1.0×10⁹ cells/L or between or between about 0.4×10⁹ cells/L and 0.7×10⁹ cells/L;
the threshold level associated with marker (4) is between or between about 0.2×10⁹ cells/L and 1.1×10⁹ cells/L or between or between about 0.4×10⁹ cells/L and 0.7×10⁹ cells/L;
the threshold level associated with marker (5) is between or between about 6.7 and 18 or between or between about 13 and 14;
the threshold level associated with marker (6) is between or between about 2.4×10¹² cells/L and 3.7×10¹² cells/L or between or between about 2.9×10¹² cells/L and 3.3×10¹² cells/L;
the threshold level associated with marker (7) is between or between about 2.1×10⁹ cells/L and 7.1×10⁹ cells/L or between or between about 2.9×10⁹ cells/L and 4.2×10⁹ cells/L;
the threshold level associated with marker (8) is between or between about 57 years and 66 years or between or between about 64 years and 66 years;
the threshold level associated with marker (9) is between or between about 22 kg/m² and 31 kg/m² or between or between about 23 kg/m² and 29 kg/m²;
the threshold level associated with marker (10) is between or between about 31 g/L and 41 g/L or between or between about 36 g/L and 40 g/L;
the threshold level associated with marker (11) is between or between about 28 IU/L and 134 IU/L or between or between about 54 IU/L and 64 IU/L;
the threshold level associated with marker (12) is between or between about 7.3 IU/L and 49 IU/L or between or between about 16 IU/L and 26 IU/L;
the threshold level associated with marker (13) is between or between about 8 IU/L and 31 IU/L or between or between about 13 IU/L and 29 IU/L;
the threshold level associated with marker (14) is between or between about 1.4 μM and 2.7 μM or between or between about 1.8 μM and 2.2 μM;
the threshold level associated with marker (15) is between or between about 3.4 μM and 23 μM or between or between about 9.4 μM and 9.6 μM;
the threshold level associated with marker (16) is between or between about 46 μM and 114 μM or between or between about 52 μM and 80 μM;
the threshold level associated with marker (17) is between or between about 0.8 mL/s and 2.0 mL/s or between or between about 1.9 mL/s and 2.0 mL/s;
the threshold level associated with marker (18) is between or between about 2.2 years and 10 years or between or between about 5.5 years and 8.3 years;
the threshold level associated with marker (19) is between or between about 4 and 11 or between or between about 4 and 5;
the threshold level associated with marker (20) is between or between about 26 days and 3205 days or between or between about 641 days and 2941 days;
the threshold level associated with marker (21) is between or between about 12 days and 2257 days or between or between about 42 days and 59 days;
the threshold level associated with marker (22) is between or between about 11 days and 493 days or between or between about 230 days and 244 days;
the threshold level associated with marker (23) is between or between about 87 days and 3356 days or between or between about 474 days and 676 days;
the threshold level associated with marker (24) is between or between about 11 days and 658 days or between or between about 51 days and 170 days;
the threshold level associated with marker (25) is between or between about 21% and 100% or between or between about 56% and 80%;
the threshold level associated with marker (26) is between or between about 2.7 mg/L and 7.7 mg/L or between or between about 3.2 mg/L and 4.6 mg/L;
the threshold level associated with marker (27) is between or between about 2.8 g/L and 75 g/L or between or between about 14 g/L and 35 g/L;
the threshold level associated with marker (28) is between or between about 150 IU/L and 319 IU/L or between or between about 181 IU/L and 319 IU/L;
the threshold level associated with marker (29) is between or between about 0.003 and 763 or between or between about 8.7 and 211;
the threshold level associated with marker (30) is between or between about 0.008 g/L and 12 g/L or between or between about 0.2 g/L and 1.0 g/L;
the threshold level associated with marker (31) is between or between about 4.3 g/L and 32 g/L or between or between about 5.3 g/L and 12 g/L;
the threshold level associated with marker (32) is between or between about 53×10⁹ cells/L and 212×10⁹ cells/L or between or between about 156×10⁹ cells/L and 181×10⁹ cells/L;
the threshold level associated with marker (33) is between or between about 132 mM and 141 mM or between or between about 136 mM and 138 mM; and/or
the threshold level associated with marker (34) is between or between about 35 ng/mL and 1300 ng/mL or between or between about 170 ng/mL and 654 ng/mL.

10. A method of predicting whether a subject will exhibit a clinical response to a T cell therapy, comprising:
(a) obtaining, for a subject having a disease or condition, a parameter of a marker or parameters of a combination of markers, wherein:
(i) the parameter or parameters are obtained prior to the subject being administered a T cell therapy comprising T cells comprising a recombinant receptor that binds to an antigen associated with the disease or condition; and
(ii) the marker or the combination of markers is selected from any of one or more subject immune profile markers, one or more subject fitness markers, one or more subject prior therapy markers, and one or more subject tumor burden markers of the subject; and
(b) predicting if the subject is likely to exhibit a clinical response to administration of the T cell therapy for treatment of the disease or condition, wherein the predicting comprises comparing the parameter or each of the parameters to an associated threshold level.

11. A method of predicting whether a subject will not exhibit a clinical response to a T cell therapy, comprising:
(a) obtaining, for a subject having a disease or condition, a parameter of a marker or parameters of a combination of markers, wherein:
(i) the parameter or parameters are obtained prior to the subject being administered a T cell therapy comprising T cells comprising a recombinant receptor that binds to an antigen associated with the disease or condition; and
(ii) the marker or the combination of markers is selected from any of one or more subject immune profile markers, one or more subject fitness markers, one or more subject prior therapy markers, and one or more subject tumor burden markers of the subject; and
(b) predicting if the subject is likely to not exhibit a clinical response to administration of the T cell therapy for treatment of the disease or condition, wherein the predicting comprises comparing the parameter or each of the parameters to an associated threshold level.

12. The method of claim 10 or claim 11, wherein the parameters of a combination of markers are obtained, and each of the parameters is compared to an associated threshold level.

13. The method of any one of claims 10-12, wherein the combination of markers comprises one or more subject immune profile markers.

14. The method of claim 13, wherein the one or more subject immune profile markers are selected from the (1) level in a blood sample of d-dimer, (2) level in a blood sample of fibrinogen, (3) level in a blood sample of lymphocytes, (4) level in a blood sample of monocytes, (5) ratio in a blood sample of monocytes to leukocytes, (6) level in a blood sample of red blood cells, and (7) level in a blood sample of white blood cells of the subject.

15. The method of claim 14, wherein the subject is predicted as likely to exhibit the clinical response if:
(i) the parameter or one or more of the parameters for markers (3), (6), and (7) are higher than an associated threshold level; or
(ii) the parameter or one or more of the parameters for markers (1), (2), (4), and (5) are lower than an associated threshold level.

16. The method of claim 14, wherein the subject is predicted as likely to not exhibit the clinical response if:
(i) the parameter or one or more of the parameters for markers (3), (6), and (7) are lower than an associated threshold level; or
(ii) the parameter or one or more of the parameters for markers (1), (2), (4), and (5) are higher than an associated threshold level.

17. The method of any one of claims 14-16, wherein:
the threshold level associated with marker (1) is between or between about 0.5 mg/L and 11 mg/L or between or between about 0.5 mg/L and 1.3 mg/L;
the threshold level associated with marker (2) is between or between about 2.2 g/L and 7.7 g/L or between or between about 4.2 g/L and 5.4 g/L;
the threshold level associated with marker (3) is between or between about 0.3×10⁹ cells/L and 1.0×10⁹ cells/L or between or between about 0.4×10⁹ cells/L and 0.7×10⁹ cells/L;
the threshold level associated with marker (4) is between or between about 0.2×10⁹ cells/L and 1.1×10⁹ cells/L or between or between about 0.4×10⁹ cells/L and 0.7×10⁹ cells/L;
the threshold level associated with marker (5) is between or between about 6.7 and 18 or between or between about 13 and 14;
the threshold level associated with marker (6) is between or between about 2.4×10¹² cells/L and 3.7×10¹² cells/L or between or between about 2.9×10¹² cells/L and 3.3×10¹² cells/L; and/or
the threshold level associated with marker (7) is between or between about 2.1×10⁹ cells/L and 7.1×10⁹ cells/L or between or between about 2.9×10⁹ cells/L and 4.2×10⁹ cells/L.

18. The method of any one of claims 10-17, wherein the combination of markers comprises one or more subject fitness markers.

19. The method of claim 18, wherein the one or more subject fitness markers are selected from the (8) age, (9) body mass index, (10) level in a blood sample of albumin, (11) level in a blood sample of alkaline phosphatase, (12) level in a blood sample of aspartate aminotransferase, (13) level in a blood sample of alanine aminotransferase, (14) level in a blood sample of direct bilirubin, (15) level in a blood sample of bilirubin, (16) level in a blood sample of creatinine, and (17) creatinine clearance of the subject.

20. The method of claim 19, wherein the subject is predicted as likely to exhibit the clinical response if:
(i) the parameter or one or more of the parameters for markers (8)-(13), (16), and (17) are higher than an associated threshold level; or
(ii) the parameter or one or more of the parameters for markers (14) and (15) are lower than an associated threshold level.

21. The method of claim 19, wherein the subject is predicted as likely to not exhibit the clinical response if:
(i) the parameter or one or more of the parameters for markers (8)-(13), (16), and (17) are lower than an associated threshold level; or
(ii) the parameter or one or more of the parameters for markers (14) and (15) are higher than an associated threshold level.

22. The method of any one of claims 19-21, wherein:
the threshold level associated with marker (8) is between or between about 57 years and 66 years or between or between about 64 years and 66 years;
the threshold level associated with marker (9) is between or between about 22 kg/m² and 31 kg/m² or between or between about 23 kg/m² and 29 kg/m²;
the threshold level associated with marker (10) is between or between about 31 g/L and 41 g/L or between or between about 36 g/L and 40 g/L;
the threshold level associated with marker (11) is between or between about 28 IU/L and 134 IU/L or between or between about 54 IU/L and 64 IU/L;
the threshold level associated with marker (12) is between or between about 7.3 IU/L and 49 IU/L or between or between about 16 IU/L and 26 IU/L;
the threshold level associated with marker (13) is between or between about 8 IU/L and 31 IU/L or between or between about 13 IU/L and 29 IU/L;
the threshold level associated with marker (14) is between or between about 1.4 μM and 2.7 μM or between or between about 1.8 μM and 2.2 μM;
the threshold level associated with marker (15) is between or between about 3.4 μM and 23 μM or between or between about 9.4 μM and 9.6 μM;
the threshold level associated with marker (16) is between or between about 46 μM and 114 μM or between or between about 52 μM and 80 μM; and/or
the threshold level associated with marker (17) is between or between about 0.8 mL/s and 2.0 mL/s or between or between about 1.9 mL/s and 2.0 mL/s.

23. The method of any one of claims 10-22, wherein the combination of markers comprises one or more subject prior therapy markers.

24. The method of claim 23, wherein the one or more subject prior therapy markers are selected from the (18) time since diagnosis, (19) number of prior therapies received, (20) time since prior autologous stem cell transplant, (21) time since prior corticosteroid therapy, (22) time since prior alkylating agent therapy, (23) time since prior topoisomerase inhibitor therapy, and (24) time since prior proteasome inhibitor therapy for the subject.

25. The method of claim 24, wherein the subject is predicted as likely to exhibit the clinical response if:
(i) the parameter or one or more of the parameters for markers (20) and (22)-(24) are higher than an associated threshold level; or
(ii) the parameter or one or more of the parameters for markers (18), (19), and (21) are lower than an associated threshold level.

26. The method of claim 24, wherein the subject is predicted as likely to not exhibit the clinical response if:
(i) the parameter or one or more of the parameters for markers (20) and (22)-(24) are lower than an associated threshold level; or
(ii) the parameter or one or more of the parameters for markers (18), (19), and (21) are higher than an associated threshold level.

27. The method of any one of claims 24-26, wherein:
the threshold level associated with marker (18) is between or between about 2.2 years and 10 years or between or between about 5.5 years and 8.3 years;
the threshold level associated with marker (19) is between or between about 4 and 11 or between or between about 4 and 5;
the threshold level associated with marker (20) is between or between about 26 days and 3205 days or between or between about 641 days and 2941 days;
the threshold level associated with marker (21) is between or between about 12 days and 2257 days or between or between about 42 days and 59 days;
the threshold level associated with marker (22) is between or between about 11 days and 493 days or between or between about 230 days and 244 days;
the threshold level associated with marker (23) is between or between about 87 days and 3356 days or between or between about 474 days and 676 days; and/or
the threshold level associated with marker (24) is between or between about 11 days and 658 days or between or between about 51 days and 170 days.

28. The method of any one of claims 10-27, wherein the combination of markers comprises one or more subject tumor burden markers.

29. The method of claim 28, wherein the one or more subject tumor burden markers are selected from the (25) percent in a blood sample of bone marrow plasma cells, (26) level in a blood sample of beta-2 microglobulin, (27) level in a blood sample of Immunoglobulin G, (28) level in a blood sample of lactate dehydrogenase, (29) ratio in a blood sample of kappa to lambda free light chain levels, (30) level in a blood sample of free light chain, (31) level in a blood sample of M-protein, (32) level in a blood sample of platelets, (33) level in a blood sample of sodium, and (34) level in a blood sample of soluble BCMA of the subject.

30. The method of claim 29, wherein the subject is predicted as likely to exhibit the clinical response if:
(i) the parameter or one or more of the parameters for markers (28), (29), (32), and (33) are higher than an associated threshold level; or
(ii) the parameter or one or more of the parameters for markers (25)-(27), (30), (31), and (34) are lower than an associated threshold level.

31. The method of claim 29, wherein the subject is predicted as likely to not exhibit the clinical response if:
(i) the parameter or one or more of the parameters for markers (28), (29), (32), and (33) are lower than an associated threshold level; or
(ii) the parameter or one or more of the parameters for markers (25)-(27), (30), (31), and (34) are higher than an associated threshold level.

32. The method of any one of claims 29-31, wherein:
the threshold level associated with marker (25) is between or between about 21% and 100% or between or between about 56% and 80%;
the threshold level associated with marker (26) is between or between about 2.7 mg/L and 7.7 mg/L or between or between about 3.2 mg/L and 4.6 mg/L;
the threshold level associated with marker (27) is between or between about 2.8 g/L and 75 g/L or between or between about 14 g/L and 35 g/L;
the threshold level associated with marker (28) is between or between about 150 IU/L and 319 IU/L or between or between about 181 IU/L and 319 IU/L;
the threshold level associated with marker (29) is between or between about 0.003 and 763 or between or between about 8.7 and 211;
the threshold level associated with marker (30) is between or between about 0.008 g/L and 12 g/L or between or between about 0.2 g/L and 1.0 g/L;
the threshold level associated with marker (31) is between or between about 4.3 g/L and 32 g/L or between or between about 5.3 g/L and 12 g/L;
the threshold level associated with marker (32) is between or between about 53×10⁹ cells/L and 212×10⁹ cells/L or between or between about 156×10⁹ cells/L and 181×10⁹ cells/L;
the threshold level associated with marker (33) is between or between about 132 mM and 141 mM or between or between about 136 mM and 138 mM; and/or
the threshold level associated with marker (34) is between or between about 35 ng/mL and 1300 ng/mL or between or between about 170 ng/mL and 654 ng/mL.

33. The method of any one of claims 1-32, wherein the combination of markers comprises the (3) level in a blood sample of lymphocytes, (22) time since prior alkylating agent therapy, and (26) level in a blood sample of beta-2 microglobulin of the subject.

34. The method of claim 33, wherein the subject is predicted as likely to exhibit the clinical response if:
(i) the parameter for marker (3) is higher than an associated threshold level;
(ii) the parameter for marker (22) is higher than an associated threshold level; or
(iii) the parameter for marker (26) is lower than an associated threshold level.

35. The method of claim 33 or claim 34, wherein the subject is predicted as likely to exhibit the clinical response if:
(i) the parameter for marker (3) is higher than an associated threshold level;
(ii) the parameter for marker (22) is higher than an associated threshold level; and
(iii) the parameter for marker (26) is lower than an associated threshold level.

36. The method of claim 33, wherein the subject is predicted as likely to not exhibit the clinical response if:
(i) the parameter for marker (3) is lower than an associated threshold level;
(ii) the parameter for marker (22) is lower than an associated threshold level; or
(iii) the parameter for marker (26) is higher than an associated threshold level.

37. The method of claim 33 or claim 36, wherein the subject is predicted as likely to not exhibit the clinical response if:
(i) the parameter for marker (3) is lower than an associated threshold level;
(ii) the parameter for marker (22) is lower than an associated threshold level; and
(iii) the parameter for marker (26) is higher than an associated threshold level.

38. The method of any one of claims 33-37, wherein:
the threshold level associated with marker (3) is between or between about 0.3×10⁹ cells/L and 1.0×10⁹ cells/L or between or between about 0.4×10⁹ cells/L and 0.7×10⁹ cells/L;
the threshold level associated with marker (22) is between or between about 11 days and 493 days or between or between about 230 days and 244 days; and/or
the threshold level associated with marker (26) is between or between about 2.7 mg/L and 7.7 mg/L or between or between about 3.2 mg/L and 4.6 mg/L.

39. The method of any one of claims 1-38, wherein the combination of markers comprise the (5) ratio in a blood sample of monocytes to leukocytes, (24) time since prior proteasome inhibitor therapy, (28) level in a blood sample of lactate dehydrogenase, and (31) level in a blood sample of M-protein of the subject.

40. The method of claim 39, wherein the subject is predicted as likely to exhibit the clinical response if:
(i) the parameter for marker (5) is lower than an associated threshold level;
(ii) the parameter for marker (24) is higher than an associated threshold level;
(iii) the parameter for marker (28) is higher than an associated threshold level; or
(iv) the parameter for marker (31) is lower than an associated threshold level.

41. The method of claim 39 or claim 40, wherein the subject is predicted as likely to exhibit the clinical response if:
(i) the parameter for marker (5) is lower than an associated threshold level;
(ii) the parameter for marker (24) is higher than an associated threshold level;
(iii) the parameter for marker (28) is higher than an associated threshold level; and
(iv) the parameter for marker (31) is lower than an associated threshold level.

42. The method of claim 39, wherein the subject is predicted as likely to not exhibit the clinical response if:
(i) the parameter for marker (5) is higher than an associated threshold level;
(ii) the parameter for marker (24) is lower than an associated threshold level;
(iii) the parameter for marker (28) is lower than an associated threshold level; or
(iv) the parameter for marker (31) is higher than an associated threshold level.

43. The method of claim 39 or claim 42, wherein the subject is predicted as likely to not exhibit the clinical response if:
(i) the parameter for marker (5) is higher than an associated threshold level;
(ii) the parameter for marker (24) is lower than an associated threshold level;
(iii) the parameter for marker (28) is lower than an associated threshold level; and
(iv) the parameter for marker (31) is higher than an associated threshold level.

44. The method of any one of claims 39-43, wherein:
the threshold level associated with marker (5) is between or between about 6.7 and 18 or between or between about 13 and 14;
the threshold level associated with marker (24) is between or between about 11 days and 658 days or between or between about 51 days and 170 days;
the threshold level associated with marker (28) is between or between about 150 JU/L and 319 IU/L or between or between about 181 IU/L and 319 IU/L; and/or
the threshold level associated with marker (31) is between or between about 4.3 g/L and 32 g/L or between or between about 5.3 g/L and 12 g/L.

45. A method of predicting whether a subject will exhibit a clinical response to a T cell therapy, comprising:
(a) obtaining, for a subject having a disease or condition, a parameter of a marker or parameters of a combination of markers, wherein:
(i) the parameter or parameters are obtained prior to the subject being administered a T cell therapy comprising T cells comprising a recombinant receptor that binds to an antigen associated with the disease or condition; and
(ii) the marker or the combination of markers is selected from the (1) level in a blood sample of d-dimer, (2) level in a blood sample of fibrinogen, (3) level in a blood sample of lymphocytes, (4) level in a blood sample of monocytes, (5) ratio in a blood sample of monocytes to leukocytes, (6) level in a blood sample of red blood cells, (7) level in a blood sample of white blood cells, (8) age, (9) body mass index, (10) level in a blood sample of albumin, (11) level in a blood sample of alkaline phosphatase, (12) level in a blood sample of aspartate aminotransferase, (13) level in a blood sample of alanine aminotransferase, (14) level in a blood sample of direct bilirubin, (15) level in a blood sample of bilirubin, (16) level in a blood sample of creatinine, (17) creatinine clearance, (18) time since diagnosis, (19) number of prior therapies received, (20) time since prior autologous stem cell transplant, (21) time since prior corticosteroid therapy, (22) time since prior alkylating agent therapy, (23) time since prior topoisomerase inhibitor therapy, (24) time since prior proteasome inhibitor therapy, (25) percent in a blood sample of bone marrow plasma cells, (26) level in a blood sample of beta-2 microglobulin, (27) level in a blood sample of Immunoglobulin G, (28) level in a blood sample of lactate dehydrogenase, (29) ratio in a blood sample of kappa to lambda free light chain levels, (30) level in a blood sample of free light chain, (31) level in a blood sample of M-protein, (32) level in a blood sample of platelets, (33) level in a blood sample of sodium, and (34) level in a blood sample of soluble BCMA of the subject; and
(b) predicting if the subject is likely to exhibit a clinical response to administration of the T cell therapy for treatment of the disease or condition based on one or more outputs of a process configured to predict, based on the marker or combination of markers, if the subject is likely to exhibit the clinical response, wherein the predicting comprises providing the parameter or parameters as input to the process.

46. A method of predicting whether a subject will not exhibit a clinical response to a T cell therapy, comprising:
(a) obtaining, for a subject having a disease or condition, a parameter of a marker or parameters of a combination of markers, wherein:
(i) the parameter or parameters are obtained prior to the subject being administered a T cell therapy comprising T cells comprising a recombinant receptor that binds to an antigen associated with the disease or condition; and
(ii) the marker or the combination of markers is selected from the (1) level in a blood sample of d-dimer, (2) level in a blood sample of fibrinogen, (3) level in a blood sample of lymphocytes, (4) level in a blood sample of monocytes, (5) ratio in a blood sample of monocytes to leukocytes, (6) level in a blood sample of red blood cells, (7) level in a blood sample of white blood cells, (8) age, (9) body mass index, (10) level in a blood sample of albumin, (11) level in a blood sample of alkaline phosphatase, (12) level in a blood sample of aspartate aminotransferase, (13) level in a blood sample of alanine aminotransferase, (14) level in a blood sample of direct bilirubin, (15) level in a blood sample of bilirubin, (16) level in a blood sample of creatinine, (17) creatinine clearance, (18) time since diagnosis, (19) number of prior therapies received, (20) time since prior autologous stem cell transplant, (21) time since prior corticosteroid therapy, (22) time since prior alkylating agent therapy, (23) time since prior topoisomerase inhibitor therapy, (24) time since prior proteasome inhibitor therapy, (25) percent in a blood sample of bone marrow plasma cells, (26) level in a blood sample of beta-2 microglobulin, (27) level in a blood sample of Immunoglobulin G, (28) level in a blood sample of lactate dehydrogenase, (29) ratio in a blood sample of kappa to lambda free light chain levels, (30) level in a blood sample of free light chain, (31) level in a blood sample of M-protein, (32) level in a blood sample of platelets, (33) level in a blood sample of sodium, and (34) level in a blood sample of soluble BCMA of the subject; and
(b) predicting if the subject is likely to not exhibit a clinical response to administration of the T cell therapy for treatment of the disease or condition based on one or more outputs of a process configured to predict, based on the marker or combination of markers, if the subject is likely to not exhibit the clinical response, wherein the predicting comprises providing the parameter or parameters as input to the process.

47. The method of claim 45 or claim 46, wherein the parameters of a combination of markers are obtained and provided as input to the process.

48. The method of any one of claims 45-47, wherein the marker is or the combination of markers comprises one or more subject immune profile markers that are selected from markers (1)-(7).

49. The method of any one of claims 45-48, wherein the marker is or the combination of markers comprises one or more subject fitness markers that are selected from markers (8)-(17).

50. The method of any one of claims 45-49, wherein the marker is or the combination of markers comprises one or more subject prior therapy markers that are selected from markers (18)-(24).

51. The method of any one of claims 45-50, wherein the marker is or the combination of markers comprises one or more subject tumor burden markers that are selected from markers (25)-(34).

52. A method of predicting whether a subject will exhibit a clinical response to a T cell therapy, comprising:
(a) obtaining, for a subject having a disease or condition, a parameter of a marker or parameters of a combination of markers, wherein:
(i) the parameter or parameters are obtained prior to the subject being administered a T cell therapy comprising T cells comprising a recombinant receptor that binds to an antigen associated with the disease or condition; and
(ii) the marker or the combination of markers is selected from any of one or more subject immune profile markers, one or more subject fitness markers, one or more subject prior therapy markers, and one or more subject tumor burden markers of the subject; and
(b) predicting if the subject is likely to exhibit a clinical response to administration of the T cell therapy for treatment of the disease or condition based on one or more outputs of a process configured to predict, based on the marker or combination of markers, if the subject is likely to exhibit the clinical response, wherein the predicting comprises providing the parameter or parameters as input to the process.

53. A method of predicting whether a subject will not exhibit a clinical response to a T cell therapy, comprising:
(a) obtaining, for a subject having a disease or condition, a parameter of a marker or parameters of a combination of markers, wherein:
(i) the parameter or parameters are obtained prior to the subject being administered a T cell therapy comprising T cells comprising a recombinant receptor that binds to an antigen associated with the disease or condition; and
(ii) the marker or the combination of markers is selected from any of one or more subject immune profile markers, one or more subject fitness markers, one or more subject prior therapy markers, and one or more subject tumor burden markers of the subject; and
(b) predicting if the subject is likely to not exhibit a clinical response to administration of the T cell therapy for treatment of the disease or condition based on one or more outputs of a process configured to predict, based on the marker or combination of markers, if the subject is likely to not exhibit the clinical response, wherein the predicting comprises providing the parameter or parameters as input to the process.

54. The method of claim 52 or claim 53, wherein the parameters of a combination of markers are obtained and provided as input to the process.

55. The method of any one of claims 52-54, wherein the combination of markers comprises one or more subject immune profile markers.

56. The method of claim 55, wherein the one or more subject immune profile markers are selected from the (1) level in a blood sample of d-dimer, (2) level in a blood sample of fibrinogen, (3) level in a blood sample of lymphocytes, (4) level in a blood sample of monocytes, (5) ratio in a blood sample of monocytes to leukocytes, (6) level in a blood sample of red blood cells, and (7) level in a blood sample of white blood cells of the subject.

57. The method of any one of claims 52-56, wherein the combination of markers comprises one or more subject fitness markers.

58. The method of claim 57, wherein the one or more subject fitness markers are selected from the (8) age, (9) body mass index, (10) level in a blood sample of albumin, (11) level in a blood sample of alkaline phosphatase, (12) level in a blood sample of aspartate aminotransferase, (13) level in a blood sample of alanine aminotransferase, (14) level in a blood sample of direct bilirubin, (15) level in a blood sample of bilirubin, (16) level in a blood sample of creatinine, and (17) creatinine clearance of the subject.

59. The method of any one of claims 52-58, wherein the combination of markers comprises one or more subject prior therapy markers.

60. The method of claim 59, wherein the one or more subject prior therapy markers are selected from the (18) time since diagnosis, (19) number of prior therapies received, (20) time since prior autologous stem cell transplant, (21) time since prior corticosteroid therapy, (22) time since prior alkylating agent therapy, (23) time since prior topoisomerase inhibitor therapy, and (24) time since prior proteasome inhibitor therapy for the subject.

61. The method of any one of claims 52-60, wherein the combination of markers comprises one or more subject tumor burden markers.

62. The method of claim 61, wherein the one or more subject tumor burden markers are selected from the (25) percent in a blood sample of bone marrow plasma cells, (26) level in a blood sample of beta-2 microglobulin, (27) level in a blood sample of Immunoglobulin G, (28) level in a blood sample of lactate dehydrogenase, (29) ratio in a blood sample of kappa to lambda free light chain levels, (30) level in a blood sample of free light chain, (31) level in a blood sample of M-protein, (32) level in a blood sample of platelets, (33) level in a blood sample of sodium, and (34) level in a blood sample of soluble BCMA of the subject.

63. The method of any one of claims 45-62, wherein the combination of markers comprises the (3) level in a blood sample of lymphocytes, (22) time since prior alkylating agent therapy, and (26) level in a blood sample of beta-2 microglobulin of the subject.

64. The method of any one of claims 45-63, wherein the combination of markers comprises the (5) ratio in a blood sample of monocytes to leukocytes, (24) time since prior proteasome inhibitor therapy, (28) level in a blood sample of lactate dehydrogenase, and (31) level in a blood sample of M-protein of the subject.

65. The method of any one of claims 45, 47-52, and 54-64, wherein the process comprises a machine learning model trained to predict, based on parameters of the marker or combination of markers, if the subject is likely to exhibit the clinical response.

66. The method of any one of claims 46-51 and 53-64, wherein the process comprises a machine learning model trained to predict, based on parameters of the marker or combination of markers, if the subject is likely to not exhibit the clinical response.

67. The method of claim 65 or claim 66, wherein the one or more outputs are outputs of, or are derived from outputs of, the machine learning model.

68. The method of any one of claims 65-67, wherein the machine learning model is trained using parameters of the marker or parameters of the combination of markers from a plurality of subjects each having a disease or condition that were each administered a T cell therapy comprising T cells comprising a recombinant receptor that binds to an antigen associated with the disease or condition.

69. The method of any one of claims 65-68, wherein the machine learning model is trained using clinical responses of the plurality of subjects following administration of the T cell therapy.

70. The method of claim 68 or claim 69, wherein the disease or condition of the plurality of subjects is the same disease or condition of the subject.

71. The method of any one of claims 68-70, wherein the antigen associated with the disease or condition of the plurality of subjects is the same antigen associated with the disease or condition of the subject.

72. The method of any one of claims 68-71, wherein the recombinant receptor of the T cell therapy of the plurality of subjects is the same recombinant receptor of the T cell therapy of the subject.

73. The method of any one of claims 68-72, wherein the T cell therapy of the plurality of subjects is an autologous T cell therapy.

74. The method of any one of claims 1-73, wherein the disease or condition is a cancer.

75. The method of any one of claims 1-74, wherein the disease or condition is a multiple myeloma.

76. The method of any one of claims 1-75, wherein the disease or condition is a relapsed/refractory multiple myeloma.

77. The method of any one of claims 1-76, wherein the antigen is a multiple myeloma-associated antigen.

78. The method of any one of claims 1-77, wherein the antigen is BCMA.

79. The method of any one of claims 1-78, wherein prior to the obtaining of the parameter or parameters, the subject has received one or more prior therapies for treating the disease or condition.

80. The method of claim 79, wherein the one or more prior therapies comprises one to three prior therapies.

81. The method of claim 79, wherein the one or more prior therapies comprises at least three prior therapies.

82. The method of any one of claims 79-81, wherein the subject has relapsed or been refractory to the most recent of the one or more prior therapies.

83. The method of any one of claims 79-82, wherein the one or more prior therapies comprises an immunomodulatory agent.

84. The method of claim 83, wherein the immunomodulatory agent is selected from thalidomide, lenalidomide, and pomalidomide.

85. The method of any one of claims 79-84, wherein the one or more prior therapies comprises a proteasome inhibitor.

86. The method of claim 85, wherein the proteasome inhibitor is selected from bortezomib, carfilzomib, and ixazomib.

87. The method of any one of claims 79-86, wherein the one or more prior therapies comprises an anti-CD38 antibody.

88. The method of claim 87, wherein the anti-CD38 antibody is or comprises daratumumab.

89. The method of any one of claims 1-88, wherein the clinical response is progression free survival of greater than 2 months, 4 months, 6 months, or 8 months.

90. The method of any one of claims 1-88, wherein the clinical response is complete response (CR).

91. The method of any one of claims 1-90, wherein the parameter or parameters are obtained within 6, 5, 4, 3, 2, or 1 month prior to when the T cell therapy is to be administered to the subject.

92. The method of any one of claims 1-91, wherein the parameter or parameters are obtained when or about when the subject is being screened for administration of the T cell therapy.

93. The method of any one of claims 1-92, wherein the parameter or parameters are obtained prior to when T cells for the T cell therapy are collected from the subject.

94. The method of any one of claims 1-93, wherein the obtaining comprises measuring the parameter or one of the more of the parameters from the subject.

95. The method of any one of claims 1-94, wherein the recombinant receptor is a chimeric antigen receptor (CAR).

96. The method of claim 95, wherein the CAR is an anti-BCMA CAR.

97. The method of claim 95 or claim 96, wherein the CAR comprises an extracellular antigen-binding domain that binds to BCMA, a transmembrane domain, and an intracellular signaling region.

98. The method of claim 97, wherein the intracellular signaling region comprises a cytoplasmic signaling domain of a CD3-zeta (CD3ζ) chain.

99. The method of claim 97 or claim 98, wherein the intracellular signaling region comprises a costimulatory signaling domain.

100. The method of claim 99, wherein the costimulatory signaling domain comprises an intracellular signaling domain of CD28, 4-1BB, or ICOS.

101. The method of claim 99 or claim 100, wherein the costimulatory signaling domain is between the transmembrane domain and the cytoplasmic signaling domain of the CD3-zeta (CD3ζ) chain.

102. The method of any of claims 97-101, wherein the transmembrane domain comprises a transmembrane domain from CD28 or CD8.

103. The method of any of claims 97-102, wherein the transmembrane domain comprises a transmembrane domain from human CD28 or CD8.

104. The method of any one of claims 97-103, wherein the CAR further comprises an extracellular spacer between the antigen-binding domain and the transmembrane domain.

105. The method of claim 104, wherein the spacer is from CD8.

106. The method of claim 104 or claim 105, wherein the spacer is a CD8alpha hinge.

107. The method of any one of claims 104-106, wherein the transmembrane domain and the spacer are from CD8.

108. The method of any one of claims 95-107, wherein the CAR comprises the sequence set forth in SEQ ID NO:38.

109. The method of any one of claims 1-108, wherein the T cell therapy is an autologous T cell therapy.

110. The method of any one of claims 1-109, wherein the T cell therapy comprises idecabtagene vicleucel cells.

111. The method of any one of claims 1-110, wherein the T cell therapy is ABECMA®.

112. The method of any one of claims 1-108, wherein the T cell therapy comprises ciltacabtagene autoleucel cells.

113. The method of any one of claims 1-108 and 112, wherein the T cell therapy is CARVYKTI™.

114. The method of any one of claims 1-113, wherein the subject is a human.

115. The method of any one of claims 3-9, 11-14, 16-19, 21-24, 26-29, 31-33, 36-39, 42-44, 46-51, 53-64, and 66-114, wherein the subject is predicted as likely to not exhibit the clinical response, and the method further comprises selecting the subject for administration of an alternative treatment or treatment regimen.

116. The method of any one of claims 1-2, 5-10, 12-15, 17-20, 22-25, 27-30, 32-35, 38-41, 44-45, 47-52, 54-65, and 67-114, wherein the subject is predicted as likely to exhibit the clinical response, and the method further comprises selecting the subject for administration of the T cell therapy.

117. The method of any one of claims 1-2, 5-10, 12-15, 17-20, 22-25, 27-30, 32-35, 38-41, 44-45, 47-52, 54-65, 67-114, and 116, wherein the method further comprises collecting T cells from the subject for producing the T cell therapy.

118. The method of claim 117, wherein the T cells are collected after the subject is predicted as likely to exhibit the clinical response.

119. The method of claim 117 or claim 118, wherein the T cells are collected by apheresis.

120. The method of any one of claims 1-119, wherein the blood sample is a whole blood sample, serum sample, or plasma sample.

121. A method of treating a disease or condition in a human subject, comprising:
(a) selecting a subject having a disease or condition for administration of a T cell therapy for treating the disease or condition, the T cell therapy comprising T cells comprising a recombinant receptor that binds to an antigen associated with the disease or condition, wherein the selecting is according to the method of any one of claims 116-120; and
(b) administering the T cell therapy to the selected subject.

122. A method of treating a disease or condition in a human subject, comprising administering a T cell therapy to a subject having a disease or condition, wherein:
the T cell therapy comprises T cells comprising a recombinant receptor that binds to an antigen associated with the disease or condition; and
the subject is selected according to the method of any one of claims 116-120 for administration of the T cell therapy.

123. A method of treating a disease or condition in a human subject, comprising administering a T cell therapy to a human subject having a disease or condition, wherein:
the T cell therapy comprises T cells comprising a recombinant receptor that binds to an antigen associated with the disease or condition; and
the subject is a subject in which prior to administration of the T cell therapy to the subject, and for a marker or a combination of markers selected from (1) level in a blood sample of d-dimer, (2) level in a blood sample of fibrinogen, (3) level in a blood sample of lymphocytes, (4) level in a blood sample of monocytes, (5) ratio in a blood sample of monocytes to leukocytes, (6) level in a blood sample of red blood cells, (7) level in a blood sample of white blood cells, (8) age, (9) body mass index, (10) level in a blood sample of albumin, (11) level in a blood sample of alkaline phosphatase, (12) level in a blood sample of aspartate aminotransferase, (13) level in a blood sample of alanine aminotransferase, (14) level in a blood sample of direct bilirubin, (15) level in a blood sample of bilirubin, (16) level in a blood sample of creatinine, (17) creatinine clearance, (18) time since diagnosis, (19) number of prior therapies received, (20) time since prior autologous stem cell transplant, (21) time since prior corticosteroid therapy, (22) time since prior alkylating agent therapy, (23) time since prior topoisomerase inhibitor therapy, (24) time since prior proteasome inhibitor therapy, (25) percent in a blood sample of bone marrow plasma cells, (26) level in a blood sample of beta-2 microglobulin, (27) level in a blood sample of Immunoglobulin G, (28) level in a blood sample of lactate dehydrogenase, (29) ratio in a blood sample of kappa to lambda free light chain levels, (30) level in serum of free light chain, (31) level in a blood sample of M-protein, (32) level in a blood sample of platelets, (33) level in a blood sample of sodium, and (34) level in a blood sample of soluble BCMA of the subject:
(i) a parameter or one or more parameters of the subject for markers (3), (6)-(13), (16), (17), (20), (22)-(24), (28), (29), (32), and (33) are higher than an associated threshold level; or
(ii) a parameter or one or more parameters of the subject for markers (1), (2), (4), (5), (14), (15), (18), (19), (21), (25)-(27), (30), (31), and (34) are lower than an associated threshold level.

124. The method of claim 123, wherein prior to administration of the T cell therapy to the subject, the subject has been determined to have:
(i) a parameter or one or more parameters for markers (3), (6)-(13), (16), (17), (20), (22)-(24), (28), (29), (32), and (33) that are higher than an associated threshold level; or
(ii) a parameter or one or more parameters for markers (1), (2), (4), (5), (14), (15), (18), (19), (21), (25)-(27), (30), (31), and (34) that are lower than an associated threshold level.

125. A method of treating a disease or condition in a human subject, comprising administering a T cell therapy to a human subject having a disease or condition, wherein:
the T cell therapy comprises T cells comprising a recombinant receptor that binds to an antigen associated with the disease or condition; and
the subject is a subject in which prior to administration of the T cell therapy to the subject, and for a marker or a combination of markers selected from any of:
one or more subject immune profile markers selected from (1) level in a blood sample of d-dimer, (2) level in a blood sample of fibrinogen, (3) level in a blood sample of lymphocytes, (4) level in a blood sample of monocytes, (5) ratio in a blood sample of monocytes to leukocytes, (6) level in a blood sample of red blood cells, and (7) level in a blood sample of white blood cells of the subject;
one or more subject prior therapy markers selected from (18) time since diagnosis, (19) number of prior therapies received, (20) time since prior autologous stem cell transplant, (21) time since prior corticosteroid therapy, (22) time since prior alkylating agent therapy, (23) time since prior topoisomerase inhibitor therapy, and (24) time since prior proteasome inhibitor therapy for the subject; and
one or more subject tumor burden markers selected from (25) percent in a blood sample of bone marrow plasma cells, (26) level in a blood sample of beta-2 microglobulin, (27) level in a blood sample of Immunoglobulin G, (28) level in a blood sample of lactate dehydrogenase, (29) ratio in a blood sample of kappa to lambda free light chain levels, (30) level in a blood sample of free light chain, (31) level in a blood sample of M-protein, (32) level in a blood sample of platelets, (33) level in a blood sample of sodium, and (34) level in a blood sample of soluble BCMA of the subject;
(i) a parameter or one or more parameters of the subject for markers (3), (6), (7), (20), (22)-(24), (28), (29), (32), and (33) are higher than an associated threshold level; or
(ii) a parameter or one or more parameters of the subject for markers (1), (2), (4), (5), (18), (19), (21), (25)-(27), (30), (31), and (34) are lower than an associated threshold level.

126. The method of claim 125, wherein prior to administration of the T cell therapy to the subject, the subject has been determined to have:
(i) a parameter or one or more parameters for markers (3), (6), (7), (20), (22)-(24), (28), (29), (32), and (33) that are higher than an associated threshold level; or
(ii) a parameter or one or more parameters for markers (1), (2), (4), (5), (18), (19), (21), (25)-(27), (30), (31), and (34) that are lower than an associated threshold level.

127. The method of any one of claims 121-126, wherein the marker is or the combination of markers comprises one or more subject immune profile markers that are selected from markers (1)-(7).

128. The method of any one of claims 121-124 and 127, wherein the marker is or the combination of markers comprises one or more subject fitness markers that are selected from markers (8)-(17).

129. The method of any one of claims 121-128, wherein the marker is or the combination of markers comprises one or more subject prior therapy markers that are selected from markers (18)-(24).

130. The method of any one of claims 121-129, wherein the marker is or the combination of markers comprises one or more subject tumor burden markers that are selected from markers (25)-(34).

131. The method of any one of claims 121-130, wherein the combination of markers comprises the (3) level in a blood sample of lymphocytes, (22) time since prior alkylating agent therapy, and (26) level in a blood sample of beta-2 microglobulin of the subject.

132. The method of claim 131, wherein:
(i) the parameter of the subject for marker (3) is higher than an associated threshold level;
(ii) the parameter of the subject for marker (22) is higher than an associated threshold level; or
(iii) the parameter of the subject for marker (26) is lower than an associated threshold level.

133. The method of claim 131 or claim 132, wherein:
(i) the parameter of the subject for marker (3) is higher than an associated threshold level;
(ii) the parameter of the subject for marker (22) is higher than an associated threshold level; and
(iii) the parameter of the subject for marker (26) is lower than an associated threshold level.

134. The method of any one of claims 121-133, wherein the combination of markers comprise the (5) ratio in a blood sample of monocytes to leukocytes, (24) time since prior proteasome inhibitor therapy, (28) level in a blood sample of lactate dehydrogenase, and (31) level in a blood sample of M-protein of the subject.

135. The method of claim 134, wherein:
(i) the parameter of the subject for marker (5) is lower than an associated threshold level;
(ii) the parameter of the subject for marker (24) is higher than an associated threshold level;
(iii) the parameter of the subject for marker (28) is higher than an associated threshold level; or
(iv) the parameter of the subject for marker (31) is lower than an associated threshold level.

136. The method of claim 134 or claim 135, wherein:
(i) the parameter of the subject for marker (5) is lower than an associated threshold level;
(ii) the parameter of the subject for marker (24) is higher than an associated threshold level;
(iii) the parameter of the subject for marker (28) is higher than an associated threshold level; and
(iv) the parameter of the subject for marker (31) is lower than an associated threshold level.

137. The method of any one of claims 121-136, wherein:
the threshold level associated with marker (1) is between or between about 0.5 mg/L and 11 mg/L or between or between about 0.5 mg/L and 1.3 mg/L;
the threshold level associated with marker (2) is between or between about 2.2 g/L and 7.7 g/L or between or between about 4.2 g/L and 5.4 g/L;
the threshold level associated with marker (3) is between or between about 0.3×10⁹ cells/L and 1.0×10⁹ cells/L or between or between about 0.4×10⁹ cells/L and 0.7×10⁹ cells/L;
the threshold level associated with marker (4) is between or between about 0.2×10⁹ cells/L and 1.1×10⁹ cells/L or between or between about 0.4×10⁹ cells/L and 0.7×10⁹ cells/L;
the threshold level associated with marker (5) is between or between about 6.7 and 18 or between or between about 13 and 14;
the threshold level associated with marker (6) is between or between about 2.4×10¹² cells/L and 3.7×10¹² cells/L or between or between about 2.9×10¹² cells/L and 3.3×10¹² cells/L;
the threshold level associated with marker (7) is between or between about 2.1×10⁹ cells/L and 7.1×10⁹ cells/L or between or between about 2.9×10⁹ cells/L and 4.2×10⁹ cells/L;
the threshold level associated with marker (8) is between or between about 57 years and 66 years or between or between about 64 years and 66 years;
the threshold level associated with marker (9) is between or between about 22 kg/m² and 31 kg/m² or between or between about 23 kg/m² and 29 kg/m²;
the threshold level associated with marker (10) is between or between about 31 g/L and 41 g/L or between or between about 36 g/L and 40 g/L;
the threshold level associated with marker (11) is between or between about 28 IU/L and 134 IU/L or between or between about 54 IU/L and 64 IU/L;
the threshold level associated with marker (12) is between or between about 7.3 IU/L and 49 IU/L or between or between about 16 IU/L and 26 IU/L;
the threshold level associated with marker (13) is between or between about 8 IU/L and 31 IU/L or between or between about 13 IU/L and 29 IU/L;
the threshold level associated with marker (14) is between or between about 1.4 μM and 2.7 μM or between or between about 1.8 μM and 2.2 μM;
the threshold level associated with marker (15) is between or between about 3.4 μM and 23 μM or between or between about 9.4 μM and 9.6 μM;
the threshold level associated with marker (16) is between or between about 46 μM and 114 μM or between or between about 52 μM and 80 μM;
the threshold level associated with marker (17) is between or between about 0.8 mL/s and 2.0 mL/s or between or between about 1.9 mL/s and 2.0 mL/s;
the threshold level associated with marker (18) is between or between about 2.2 years and 10 years or between or between about 5.5 years and 8.3 years;
the threshold level associated with marker (19) is between or between about 4 and 11 or between or between about 4 and 5;
the threshold level associated with marker (20) is between or between about 26 days and 3205 days or between or between about 641 days and 2941 days;
the threshold level associated with marker (21) is between or between about 12 days and 2257 days or between or between about 42 days and 59 days;
the threshold level associated with marker (22) is between or between about 11 days and 493 days or between or between about 230 days and 244 days;
the threshold level associated with marker (23) is between or between about 87 days and 3356 days or between or between about 474 days and 676 days;
the threshold level associated with marker (24) is between or between about 11 days and 658 days or between or between about 51 days and 170 days;
the threshold level associated with marker (25) is between or between about 21% and 100% or between or between about 56% and 80%;
the threshold level associated with marker (26) is between or between about 2.7 mg/L and 7.7 mg/L or between or between about 3.2 mg/L and 4.6 mg/L;
the threshold level associated with marker (27) is between or between about 2.8 g/L and 75 g/L or between or between about 14 g/L and 35 g/L;
the threshold level associated with marker (28) is between or between about 150 IU/L and 319 IU/L or between or between about 181 IU/L and 319 IU/L;
the threshold level associated with marker (29) is between or between about 0.003 and 763 or between or between about 8.7 and 211;
the threshold level associated with marker (30) is between or between about 0.008 g/L and 12 g/L or between or between about 0.2 g/L and 1.0 g/L;
the threshold level associated with marker (31) is between or between about 4.3 g/L and 32 g/L or between or between about 5.3 g/L and 12 g/L;
the threshold level associated with marker (32) is between or between about 53×10⁹ cells/L and 212×10⁹ cells/L or between or between about 156×10⁹ cells/L and 181×10⁹ cells/L;
the threshold level associated with marker (33) is between or between about 132 mM and 141 mM or between or between about 136 mM and 138 mM; and/or
the threshold level associated with marker (34) is between or between about 35 ng/mL and 1300 ng/mL or between or between about 170 ng/mL and 654 ng/mL.

138. The method of any one of claims 121-137, wherein the T cell therapy comprises between at or about 5×10⁷ recombinant receptor-comprising T cells and at or about 1×10⁹ recombinant receptor-comprising T cells or between at or about 1×10⁹ recombinant receptor-comprising T cells and at or about 1×10⁹ recombinant receptor-comprising T cells.

139. The method of any one of claims 121-138, wherein the T cell therapy comprises at or about 4.5×10⁸ recombinant receptor-comprising T cells.

140. The method of any one of claims 121-139, wherein the T cell therapy is administered by an intravenous infusion.

141. The method of any one of claims 121-140, wherein the T cell therapy is an autologous T cell therapy.

142. The method of any one of claims 121, 122, 124, and 126-141, wherein the subject is subject to apheresis to collect T cells for the T cell therapy, and wherein the selection and/or determination occurs prior to the apheresis.

143. The method of any one of claims 121, 122, 124, and 126-142, wherein the selection and/or determination is within 6, 5, 4, 3, 2, or 1 month prior to when the T cell therapy is administered to the subject.

144. The method of any one of claims 121, 122, 124, and 126-143, wherein the selection and/or determination occurs at screening of the subject for administration of the T cell therapy.

145. The method of any one of claims 121-144, wherein the disease or condition is a hematologic disease.

146. The method of any one of claims 121-145, wherein the disease or condition is a multiple myeloma.

147. The method of any one of claims 121-146, wherein the antigen associated with the disease or condition is human BCMA.

148. The method of any one of claims 121-147, wherein the T cell therapy is a CAR T cell therapy.

149. The method of any one of claims 121, 122, 124, and 126-148, wherein the method further comprises administering a bridging therapy to the subject, wherein the bridging therapy is administered to the subject between the selection and/or determination and the administration of the T cell therapy.
